(19) United States
(12) Patent Application Publication
(10) Pub. No.: US 2023/0128981 A1
(43) Pub. Date: Apr. 27, 2023

(54) CD24-LOADED VESICLES FOR TREATMENT OF CYTOKINE STORM AND OTHER CONDITIONS

(71) Applicant: FLORIDA STATE UNIVERSITY RESEARCH FOUNDATION, INCORPORATED, TALLAHASSEE, FL (US)

(72) Inventors: Zucai Suo, Tallahassee, FL (US); Mangesh D. Hade, Tallahassee, FL (US)

(21) Appl. No.: 18/047,741

(22) Filed: Oct. 19, 2022

Related U.S. Application Data
(60) Provisional application No. 63/262,759, filed on Oct. 20, 2021.
(60) Provisional application No. 63/367,492, filed on Jul. 1, 2022.

(51) Int. Cl. C07K 14/705; A61K 47/64

(52) U.S. Cl. CPC C07K 14/70596; A61K 47/64

(57) ABSTRACT

The invention concerns a vesicle, such as an extracellular vesicle or lipid vesicle, that has been loaded with a cargo molecule comprising a CD24 molecule, covalently or non-covalently coupled to a cell penetrating polypeptide (resulting in a “binding complex”), and the cargo molecule or binding complex has been internalized by the vesicle, associated with the vesicle, or a combination thereof. Advantageously, the CD24 molecule may have extracellular amino acids that become displayed on the outer surface of the vesicle upon loading. Other aspects of the invention concern a vesicle loaded with cargo molecule comprising a CD24 molecule, a method for loading a vesicle with a cargo molecule comprising a CD24 molecule, and a method for delivering a CD24 molecule into a cell in vitro or in vivo, to control inflammation such as for treatment, prevention, or delay of onset of cytokine storm.

### CROSS-REFERENCE TO RELATED APPLICATIONS

This application claims the benefit of and priority to co-pending U.S. Provisional Patent Application Nos. 63/262,759, filed on Oct. 20, 2021, and 63/367,492, filed on Jul. 1, 2022, the contents of which are incorporated by reference herein in their entireties.

### CROSS REFERENCE TO SEQUENCE LISTING

The peptides described herein are referred to by sequence identifier numbers (SEQ ID NO). The SEQ ID NOs correspond numerically to the sequence identifiers <400>1, <400>2, etc. The sequence listing in written computer readable format (CRF) as a text file filed concurrently with this application is incorporated by reference in its entirety.

### BACKGROUND

A cytokine storm, also called hypercytokinemia, is a life-threatening physiological reaction in humans and other animals in which the immune system causes an uncontrolled and excessive release of pro-inflammatory cytokines (Fajgenbaum D C et al., “Cytokine Storm, N Engl J Med, 2020, Dec. 3, 383 (23):2255-2273; and Ye Q et al., “The pathogenesis and treatment of the ‘Cytokine Storm’ in COVID-19, Journal of Infection, 2020, 80:6-7-613). Inflammation is an innate immune response to infection and tissue injury. Although cytokines are part of the body's normal immune response to infection, their sudden release in large quantities can cause multisystem organ failure and even death. The elevated levels of circulating cytokines and immune cell hyper-activation can be triggered by various therapies, pathogens, cancers, autoimmune conditions, and monogenic disorders.

Several therapeutic strategies have been considered to address cytokine storms, such as antibody therapies with tocilizumab, sarilumab, and siltuximab and blood-purification techniques (BPT), including therapeutic plasma exchange (TPE), absorption, perfusion, and blood plasma filtration, among others; however, there remains a need for timely control of cytokine storms to avoid the associated multisystem organ failure and deaths (Guo J et al., “The Artificial-Liver Blood-Purification System Can Effectively Improve Hypercytokinemia for COVID-19”, Front Immunol., 2020; 11: 586073; Mehta P et al., “COVID-19: consider cytokine storm syndromes and immunosuppression”, Lancet, 2020, 395 (10229):1033-4; Hirasawa H et al., “Continuous hemodiafiltration with a cytokine-adsorbing hemofilter for sepsis,” Blood Purif, 2012, 34 (2):164-70; Shiga H et al., “Continuous Hemodiafiltration with a Cytokine-Adsorbing Hemofilter in Patients with Septic Shock: A Preliminary Report, Blood Purif, 2014, 38 (3-4):211-8; Gucyetmez B et al., “Therapeutic plasma exchange in patients with COVID-19 pneumonia in intensive care unit: a retrospective study”, Crit Care, 2020, 24 (1):492; Esmaeili Vardanjani A et al., “Early Hemoperfusion for Cytokine Removal May Contribute to Prevention of Intubation in Patients Infected with COVID-19”, Blood Purif, 2020, 1-4.

Danger-associated molecular patterns (DAMPs) are host-derived molecules that are released from damaged or dying cells and can initiate and perpetuate a non-infectious inflammatory response by interacting with pattern recognition receptors (PRRs) (Fan X et al., “Changes of Damage Associated Molecular Patterns in COVID-19 Patients”, Infectious Diseases & Immunity, April 2021, 1 (1):20-27; Zindel J et al., “DAMPs, PAMPs, and LAMPs in immunity and sterile inflammation, Annu Rev Pathol, 2020, 15:493-518). It has been proposed that CD24 is an innate checkpoint against the inflammatory response to tissue injuries or DAMPS. CD24Fc comprises the non-polymorphic regions of CD24 coupled to the Fc region of human immunoglobulin G1 (IgG1). CD24Fc is reported to bind DAMPs, thereby preventing the interaction of DAMPs with toll-like receptors (TLRs) and inhibiting both nuclear factor-kappa B (NFkB) activation and secretion of inflammatory cytokines, and is therefore being tested as an immunomodulator drug in COVID-19 patients in a phase 3 clinical trial (NCT04317040).

### SUMMARY

The invention relates to the use of vesicles such as exosomes, liposomes, and other vesicles, loaded with CD24 molecules, as cargo, for delivery of the CD24 molecules. The delivered CD24 molecules retain their function as a biological immunomodulator, and may be used for treating hyper-inflammation and conditions such as hypercytokinemia (also known as cytokine storm) in a human or non-human animal subject.

Hypercytokinemia and other conditions of hyper-inflammation that may be treated using the vesicles and methods of the invention can be caused by various infectious and non-infectious etiologies, especially viral respiratory infections such as H5N1 influenza, SARS-CoV-1, and SARS-CoV-2 (causative agent for COVID-19 pandemic). Other causative agents include, for example, the Epstein-Barr virus, cytomegalovirus, and group A streptococcus, non-infectious conditions such as graft-versus-host disease, and therapies such as chimeric antigen receptor (CAR)-expressing T cell (CAR-T cell) cell therapy.

One aspect of the invention concerns a method for loading a vesicle, such as an extracellular vesicle (EV) or lipid vesicle (LV), with a cargo molecule comprising CD24, or a biologically active fragment or variant of CD24, the method comprising contacting the vesicle with a binding complex, wherein the binding complex comprises the cargo molecule and a cell penetrating polypeptide (CPP) covalently or non-covalently coupled to the cargo molecule, and wherein the binding complex becomes internalized by the vesicle, associated with the vesicle, or a combination thereof to produce a loaded vesicle. The CPP may be covalently or non-covalently coupled to the CD24 of the cargo molecule or, in cases in which the cargo molecule includes an additional component coupled to the CD24 (e.g., such as in the case of a fusion protein), the CPP may be coupled to the CD24 or the additional component, or coupled to both.

Another aspect of the invention concerns a loaded vesicle, such as that produced by the aforementioned loading method. In some embodiments, the loaded vesicle comprises a binding complex, wherein the binding complex comprises a cargo molecule and a CPP covalently or non-covalently coupled to the cargo molecule, wherein the cargo molecule comprises CD24, or a biologically active fragment or variant of CD24, and wherein the binding complex has been internalized by the vesicle, associated with the vesicle, or a combination thereof to produce a loaded vesicle.

Another aspect of the invention concerns a loaded vesicle comprising a cargo molecule comprising CD24, or a biologically active fragment or variant of CD24, and a CPP. The CD24 cargo may still be covalently or non-covalently coupled to a CPP (together referred to as a binding complex), wherein the binding complex has been internalized within the vesicle, or is associated with the vesicle membrane; or the CD24 cargo may be uncoupled from the CPP once the CD24 cargo has been internalized within the vesicle or is associated with the vesicle membrane (i.e., the components of the binding complex have become physically separated, no longer forming the complex).

In some embodiments, the loaded vesicle comprising an outer layer having an outer surface; a lumen surrounded by the outer layer; and a cargo molecule comprising CD24, or a biologically active fragment or variant of CD24, wherein the CD24, or biologically active fragment or variant includes extracellular amino acids displayed on the outer surface of the vesicle and other amino acids are bound inside the outer layer and/or presented in the lumen of the vesicle. Vesicles may have a single membrane layer or have multiple layers of membranes.

Another aspect of the invention concerns a method for delivering CD24, or a biologically active fragment or variant of CD24 (i.e., CD24 cargo), into a cell in vitro or in vivo, comprising administering a loaded vesicle of the invention, wherein the loaded vesicle is internalized into the cell, and wherein the loaded vesicle contains or carries the CD24 cargo and a CPP. The CD24 cargo and CPP may still be coupled at the time of administration of the loaded vesicles to cells or the CD24 cargo and CPP may be in an uncoupled condition. The CD24 delivery method may be used to provide one or more biological activities possessed by the CD24 cargo of the vesicle. When administered to a human or non-human animal subject (in vivo administration), the CD24 delivery method may be used to reduce an inflammatory response in the subject.

In some embodiments of the CD24 delivery method, the loaded vesicle is administered to the subject to treat, prevent, or delay the onset of hyper-inflammation in the subject. In some embodiments, the loaded vesicle is administered to the subject to treat, prevent, or delay the onset of cytokine storm in the subject. The cytokine storm may be caused by an infectious agent (e.g., SARS-CoV-2 or a variant thereof), or may be a monogenic disorder or autoimmune disorder (e.g., auto-inflammatory disorder, primary or secondary hemophagocytic lymphohistiocytosis (HLH)), or may have an iatrogenic cause (e.g., CAR-T cell therapy, blinatumomab, other T-cell engaging immunotherapy, or gene therapy). In some embodiments, the cytokine storm is caused by cancer, one or more cancer therapies (e.g., chemotherapy, immunotherapy, radiotherapy), or both.

Advantageously, the methods of the invention allow one to load natural or engineered, human or non-human animal, CD24 molecules into vesicles such as exosomes or liposomes, without being cleaved or modified by extracellular enzymes, bound by host proteins, or neutralized by host immune responses.

### BRIEF DESCRIPTION OF THE DRAWINGS

The patent or application file contains at least one drawing executed in color. Copies of this patent or patent application publication with color drawing(s) will be provided by the Patent and Trademark Office upon request and payment of the necessary fee.

FIGS. 1A and 1B. The cell-penetrating peptide (CPP) R9 can load a protein cargo into exosomes as shown by total internal reflection fluorescence (TIRF) microscopy. FIG. 1A is a TIRF image showing the loaded exosomes after the two-hour incubation of the exosomes with the purified protein GST-CD24-R9-Cy3 at 37° C. The N-terminal GST tag in GST-CD24-R9-Cy3 denotes the glutathione S-transferase tag. FIG. 1B is a magnified TIRF image of an individual exosome.

FIGS. 2A and 2B. The CPP R9 can load a protein cargo CD24 into exosomes as shown by confocal microscopy. FIG. 2A shows a confocal image of the purified exosomes after the two-hour loading of the purified protein CD24-R9-Cy5 at 37° C. FIG. 2B is a magnified confocal image of individual exosomes.

FIGS. 3A-1, 3A-2, 3B-1, 3B-2, 3C-1, 3C-2, 3D-1, and 3D-2. The CPP R9 can load a protein cargo onto the membrane of exosomes with the extracellular amino acid residues of human CD24 displayed on the outside surface of the exosomes. FIGS. 3A-1 is a TIRF image of the loaded exosomes at the Cy5 channel. The exosomes were first incubated with the complex of the Cy5-labeled anti-CD24 antibody and the recombinant protein GST-CD24-R9-Cy3 for two hours at 37° C. The loaded exosomes were then washed to get rid of any free protein molecules before being imaged via TIRF microscopy. FIG. 3B-1 is a TIRF image of the loaded exosomes from (FIG. 3A-1) at the Cy3 channel. FIG. 3C-1 is a TIRF image of the exosomes at the Cy5 channel. The exosomes were first incubated with GST-CD24-R9-Cy3 for two hours at 37° C. After washing off any free GST-CD24-R9-Cy3 molecules, the loaded exosomes were incubated with the Cy5-labeled anti-CD24 antibody for two hours before being washed again to get rid of any unbound antibody molecules. FIG. 3D-1 is a TIRF image of the exosomes from (FIG. 3C-1) at the Cy3 channel. FIGS. 3A-2, 3B-2, 3C-2, and 3D-2 are insets showing the magnified TIRF image of an individual exosome.

FIG. 4. Cy5 standard curve. Commercial Cyanine5 maleimide was used to build the curve.

FIG. 5. CD24-R9-Cy5 was loaded into exosomes in a time-dependent manner. The purified protein CD24-R9-Cy5 was incubated with exosomes (1×10⁸ particles/mL) for increasing amounts of time. After washing off any unloaded CD24-R9-Cy5, the Cy5 fluorescent intensity was measured as relative fluorescent units (RFUs) and the CD24-R9-Cy5 concentration was determined from the standard curve in FIG. 4.

FIG. 6. GST-CD24-R9 was loaded onto the membrane of exosomes in a time-dependent manner. The purified protein GST-CD24-R9-Cy3 was incubated with exosomes (1×10⁸ particles/mL) for increasing amounts of time. After washing off any unloaded GST-CD24-R9-Cy3, the exosomes were incubated with the Cy5-labeled anti-CD24 antibody. After washing off the unbound antibody, the Cy5 fluorescent intensity of the exosome/antibody complex was measured as RFU for the determination of the concentration of GST-CD24-R9-Cy3 on the exosome surface based on the standard curve in FIG. 4.

FIG. 7. Cellular uptake of exosomes loaded with a protein cargo. FIG. 7 shows bright field, DAPI, Cy3, and superimposed images of human primary dermal fibroblast cells after the four-hour incubation at 37° C. with the exosomes loaded with the recombinant protein GST-CD24-R9-Cy3. The internalization of the loaded exosomes into human fibroblast cells was confirmed using confocal microscopy. Scale bars are 50 μm.

FIG. 8. Human primary dermal fibroblasts treated with the exosomes loaded with CD24-R9 showed higher proliferation than other treatments in MTS cell proliferation assays. Human primary dermal fibroblasts were seeded at a density of 5×10⁴ cells/well into 96 well plates and exposed to the indicated treatments. The exosome concentration in each case was 1×10⁸ particles/well. The MTS assay was performed to assess cell proliferation after t=24, 48 and 72 hours under normal growth conditions, as per manufacturer's instructions. Absorbance (Abs) was read at 450 nm. Values were represented of mean±SD from four independent experiments. Statistical significance was derived by two-way ANOVA followed by Bonferroni's posttest (*** denotes p<0.001).

FIGS. 9A and 9B. Exosomes loaded with CD24-R9 increased the invasion of human primary dermal fibroblasts in cell invasion assays. Primary dermal fibroblasts were seeded at density 1×10⁶ cells/mL onto 24 well plates and exposed to the indicated treatments, as shown in FIG. 9A. The exosome concentration in each case except the control was 1×10⁸ particles/ml. Cell invasion assays were performed after t=48 h under normal growth conditions, as per manufacturer's instructions. FIG. 9B shows quantitation of the cell invasion assays in FIG. 9A. Absorbance (Abs) was read at 560 nm. Values were represented as mean±SD from four independent experiments. Statistical significance was derived by one-way ANOVA followed by Dunnett's test (*** p<0.001).

FIGS. 10A and 10B. The CPP R9 can load a protein cargo GST-CD24 into liposomes. Liposomes were first incubated with the purified GST-CD24-R9-Cy3 protein for five hours at room temperature and then washed with PBS buffer for three times and filtered. FIG. 10A is a TIRF image of the loaded and washed liposomes emitting strong Cy3 fluorescence at the Cy3 channel. FIG. 10B is an inset showing a magnified TIRF image of a single liposome.

FIGS. 11A and 11B. The CPP R9 can load a CD24 protein cargo into liposomes. FIG. 11A is a confocal image of liposomes after a five-hour incubation at room temperature with the purified CD24-R9-Cy5 protein. FIG. 11B is an inset showing a magnified confocal image of a single liposome.

FIGS. 12A, 12A-1, 12B, 12B-1, 12C, 12C-1, 12D, and 12D-1. The CPP R9 can load a GST-CD24 protein cargo onto both the surface of, and inside, individual liposomes. FIG. 12A is a TIRF image of the loaded liposomes after their five-hour incubation at room temperature with the complex of Cy5-labeled anti-CD24 antibody and recombinant protein GST-CD24-R9-Cy3 at the Cy5 channel. FIG. 12A-1 is an inset showing a magnified TIRF image of an individual liposome. FIG. 12B is a TIRF image of the liposomes from FIG. 12A at the Cy3 channel. FIG. 12B-1 is an inset showing a magnified TIRF image of an individual liposome. FIG. 12C is a TIRF image of loaded liposomes at the Cy5 channel. The liposomes were first incubated with GST-CD24-R9-Cy3 for five hours at room temperature. After washing out unincorporated GST-CD24-R9-Cy3, the loaded liposomes were incubated with the Cy5-labeled anti-CD24 antibody. Any unbound antibody molecules were washed out before imaging. FIG. 12C-1 is an inset showing a magnified TIRF image of an individual liposome. FIG. 12D is a TIRF image of the liposomes from FIG. 12C at Cy3 channel. FIG. 12D-1 is an inset showing a magnified TIRF image of an individual liposome.

FIG. 13. Sequence alignment of the isoforms of human signal transducer CD24. An amino acid sequence comparison indicates that all CD24 isoforms evaluated have an identical transmembrane region except isoform A0A087WU21. The predicted putative transmembrane regions of CD24 are highlighted in yellow color (amino acid sequence ALQSTASLFVVSLSLLHLYS). Conserved sequences are highlighted in grey color and are considered to interact with other cellular or membrane proteins. “*” indicates conserved residues among all five homologs while “.” shows conserved residues but with one mutation in one of five homologs.

FIGS. 14A-14E. Hydropathy plots of the isoforms of human signal transducer CD24. FIG. 14A: P25063 (predicted transmembrane region: residues 60-80); FIG. 14B: P25063-2 (predicted transmembrane region: residues 102-122); FIG. 14C: A0A087WU87 (predicted transmembrane region: residues 68-88); FIG. 14D: A0A087WW33 (predicted transmembrane region: residues 100-120). FIG. 14E: A0A087WU21.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

The invention relates to the use of vesicles such as exosomes, liposomes, and other extracellular vesicles (EVs) and lipid vesicles (LVs), loaded with CD24 molecules, as cargo, for delivery of the CD24 molecules to cells in vitro or to cells of a human or non-human animal subject in vivo. Advantageously, the methods of the invention allow one to load natural or engineered, human or non-human animal, CD24 polypeptides, or biologically active fragments or variants thereof, into vesicles such as exosomes or liposomes, without being cleaved or modified by extracellular enzymes, bound by host proteins, or neutralized by host immune responses.

The delivered CD24 molecules can retain their biological function as an immunomodulator and may be used for treating hyper-inflammation and conditions such as hypercytokinemia (also known as cytokine storm) in a human or non-human animal subject. The hypercytokinemia can be caused by various infectious and non-infectious etiologies, especially viral respiratory infections such as H5N1 influenza, SARS-CoV-1, and SARS-CoV-2 (causative agent for COVID-19 pandemic). Other causative agents include, for example, the Epstein-Barr virus, cytomegalovirus, and group A streptococcus, non-infectious conditions such as graft-versus-host disease, and therapies such as chimeric antigen receptor (CAR)-expressing T cell (CAR-T cell) cell therapy.

One aspect of the invention concerns a method for loading a vesicle, such as an EV or LV, with a cargo molecule comprising CD24, or a biologically active fragment or variant of CD24, the method comprising contacting the vesicle with a binding complex, wherein the binding complex comprises the cargo molecule and a cell penetrating polypeptide (CPP) covalently or non-covalently coupled to the cargo molecule, and wherein the binding complex becomes internalized by the vesicle, associated with the vesicle, or a combination thereof to produce a loaded vesicle.

The CPP may be covalently or non-covalently coupled to the CD24 of the cargo molecule or, in cases in which the cargo molecule includes an additional component coupled to the CD24 (e.g., such as in the case of a fusion protein), the CPP may be coupled to the CD24 or the additional component, or coupled to both.

The CD24 can be a polypeptide that is synthesized or is recombinantly produced from a host cell. The CD24 polypeptide may be human CD24 (e.g., the canonical predominant amino acid sequence or an isoform), or a non-human animal CD24 (e.g., mouse), or a biologically active fragment or variant of any of the foregoing.

Human or non-animal CD24, or a biologically active fragment or biologically active variant of CD24, can be either covalently coupled or non-covalently bound to a CPP. If it is for covalent coupling, the CPP and the CD24, or a biologically active fragment or variant thereof, can be genetically fused together and expressed as a fusion protein (CD24-CPP) in host cells such as E. coli, yeast, plants, insect cells, or mammalian cells before purification via column chromatography. If it is for non-covalent binding with a CPP, the CD24, or a biologically active fragment or variant thereof, can be expressed in host cells such as E. coli, yeast, plants, insect cells, or mammalian cells and then purified via column chromatography. Second, the purified fusion protein or the purified non-covalent complex CD24/CPP can be incubated with and then enter and display on the membrane surface of vesicles (“loaded vesicles”). Next, if the linkage between the CD24, or biologically active fragment or variant thereof, and CPP is a disulfide bond, the loaded vesicles can be incubated with a reducing agent such as dithiothreitol (DTT) to break the disulfide linkage and free the CD24 or fragment or variant molecules on the outer surface of the vesicle membrane. If the covalent coupling between CPP and the CD24 or biologically active fragment or variant is via a photo-cleavable bond, it can be broken by exposing the loaded vesicles with appropriate wavelength lights, which will free the CD24 or biologically active fragment or variant within the membrane of the vesicles. Finally, the loaded vesicles can be administered to patients.

In some embodiments, the CPP is covalently coupled to the cargo molecule by a disulfide bond, an amide bond, a chemical bond formed between a sulfhydryl group and a maleimide group, a chemical bond formed between a primary amine group and an N- Hydroxysuccinimide (NHS) ester, a chemical bond formed via Click chemistry, or other covalent linkage.

In some embodiments, the CPP is covalently coupled to the cargo molecule by a cleavable linker.

In some embodiments, the cleavable linker is a photo-cleavable linker.

In some embodiments, the cargo molecule includes a further molecule fused directly or indirectly to the CD24 or the biologically active fragment or variant thereof. In some embodiments, the further molecule is selected from among a small molecule (e.g., a drug, a fluorophore, a luminophore), macromolecule such as polyimide, proteins (e.g., enzymes, membrane-bound proteins), polypeptide (natural or modified), nucleic acid (e.g., natural, damaged or chemically modified DNA, DNA plasmid or vector, telomere, DNA quadruplex, DNAzyme, DNA-like molecule, antisense oligonucleotide, locked nucleic acid, threose nucleic acid, peptide nucleic acid (PNA), single or double-stranded nucleic acid, natural, damaged or chemically modified RNA, enzymatic catalytic RNA, RNAzyme, ribozyme, non-coding RNA (ncRNA) such as microRNA (miRNA), small nuclear RNA (snRNA), interfering RNA such siRNA or shRNA, single guide RNA for a gene editing enzyme (e.g., Cas9), messenger RNA (mRNA), transfer RNA (tRNA), and ribosomal RNA (rRNA)), antibody or antibody-fragment, lipoprotein, carbohydrate, or glycoprotein.

In some embodiments, the vesicle is an EV such as an exosome (or small EV), mitovesicle, apoptotic body, microvesicle, microparticle, ectosome, or oncosome. In some embodiments, the vesicle is an EV obtained from a mature cell. In some embodiments, the vesicle is an EV obtained from a stem cell or progenitor cell. In some embodiments, the EV is obtained from a human or non-human animal mesenchymal stem cell. In other embodiments, the vesicle is an LV, such as a liposome, lipid nanoparticle, lipid droplet, micelle, reverse micelle, or lipid-polymer hybrid nanoparticle.

In some embodiments, in addition to the CD24 molecule, the cargo molecule also includes a detectable agent or medical imaging agent, or be attached to a detectable or medical imaging agent, such as a fluorescent compound (e.g., a fluorophore) to serve as a marker, dye, tag, or reporter.

In some embodiments, the vesicle also includes a targeting agent that targets the vesicle to a cell type, organ, or tissue (e.g., lung cells, cancer cells, neural cells of the central nervous system or peripheral nervous system, or muscle cells).

In some embodiments, the CPP is one listed in Table 2. In some embodiments, the CPP is selected from among the following: Tat, Antennapedia, VP22, CaP, YopM, Artificial protein B1, 30Kc19, engineered +36 GFP, and naturally supercharged human protein.

The vesicle loading method may further include, as a step in the method, the step of coupling the CPP to the cargo molecule prior to contacting the vesicle with the binding complex.

Another aspect of the invention concerns a loaded vesicle, such as a vesicle produced by the aforementioned loading method. In some embodiments, the loaded vesicle comprises a binding complex, wherein the binding complex comprises a cargo molecule and a CPP covalently or non-covalently coupled to the cargo molecule, wherein the cargo molecule comprises CD24, or a biologically active fragment or variant of CD24, and wherein the binding complex has been internalized by the vesicle, associated with the vesicle, or a combination thereof.

Another aspect of the invention concerns a loaded vesicle comprising an outer layer having an outer surface; a lumen surrounded by the outer layer; and a cargo molecule comprising CD24, or a biologically active fragment or variant of CD24, wherein the CD24, or biologically active fragment or variant includes extracellular amino acids displayed on the outer surface of the vesicle and other amino acids are bound inside the outer layer and/or presented in the lumen of the vesicle.

Another aspect of the invention concerns a method for delivering CD24, or a biologically active fragment or variant of CD24, into a cell in vitro or in vivo, comprising administering a loaded vesicle of the invention, wherein the loaded vesicle is internalized into the cell. The CD24 delivery method may be used to provide one or more biological activities possessed by the CD24 cargo of the vesicle. When administered to a human or non-human animal subject (in vivo administration), the CD24 delivery method may be used to reduce, alleviate, eliminate, prevent, or delay onset of an inflammatory response in the subject.

In some embodiments of the CD24 delivery method, the loaded vesicle is administered to the subject to treat, prevent, or delay the onset of hyper-inflammation in the subject. In some embodiments, the loaded vesicle is administered to the subject to treat, prevent, or delay the onset of cytokine storm in the subject. The cytokine storm may be caused by an infectious agent (e.g., SARS-CoV-2 or a variant thereof) or may be a monogenic disorder or autoimmune disorder (e.g., auto-inflammatory disorder, primary or secondary hemophagocytic lymphohistiocytosis (HLH)), or may have an iatrogenic cause (e.g., CAR-T cell therapy, blinatumomab, other T-cell engaging immunotherapy, or gene therapy). In some embodiments, the cytokine storm is caused by cancer, one or more cancer therapies (e.g., chemotherapy, immunotherapy, radiotherapy), or both.

In some embodiments, the loaded vesicle is administered to a human or non-human animal subject to treat, prevent, or delay onset of inflammation from danger-associated molecular patterns, or inflammation from septic injuries, as described in U.S. Pat. No. 8,637,013 “Treatment of drug-related side effect and tissue damage by targeting the CD24-HMGB1-Siglec10 axis” (Liu Y et al., issued Jan. 28, 2014, which is incorporated herein by reference in its entirety).

Currently, CD24Fc, a fusion of the N-terminal 56 amino acid residues of human CD24 and the Fc region of human IgG1, is in Phase III clinical trial by Merck to treat middle and late stages of SARS-CoV2 infection and has been found to significantly reduce death. There is also a report that exosomes containing a few CD24 molecules during exosome biogenesis were used to successfully cure 30 out of 30 SARS-CoV2 infected patients in an Israeli hospital (yahoo.com/news/israeli-covid-drug-cured-30-191709164.html). The invention can allow much higher copy number of engineered CD24 molecules to be first loaded into vesicles such as EVs and LVs, and then allow patients to breath or inhale the CD24-loaded vesicles in as a gas to treat hypercytokinemia and rebalance their immune systems. This is better than the direct administration of CD24Fc into patients since the vesicles will protect the CD24 molecules (full-length or biologically active fragments thereof) from degradation/modification by extracellular enzymes or neutralization by host immune responses.

The vesicles loaded with CD24, or biologically active fragments or variants thereof, can be directly used as therapeutic agents for treating hypercytokinemia. Optionally, vesicles loaded with CD24, or biologically active fragments or variants thereof, can be used in combination with other immuno-modulators in order to achieve potential therapeutic or prophylactic synergy.

As indicated above, one aspect of the invention concerns a method for loading a vesicle, such as an EV or LV, with a cargo molecule comprising CD24, or a biologically active fragment or variant of CD24, the method comprising contacting the vesicle with a binding complex composed of at least the cargo molecule and a CPP covalently or non-covalently coupled to the cargo molecule, wherein the binding complex becomes internalized by, or associated with, the vesicle.

Each vesicle has a core surrounded by a membrane having at least one lipid layer (e.g., at least one lipid monolayer or at least one lipid bilayer), and the “binding complex” may be internalized and contained within the core of the vesicle, or be bound and/or embedded within the encapsulating membrane(s).

Examples 1-6 herein demonstrate that CPPs can be used to load cargos comprising CD24 into EVs, how much of the CD24 is loaded, and how the loaded CD24 protein is arranged in and on the vesicle (e.g., extracellular residues displayed on the outside surface of the vesicles while the rest of the residues of CD24 are either bound inside the membrane or presented in the lumen of the vesicles). Examples 7 and 8 demonstrate that CD24-loaded EVs can fuse with and deliver the CD24 cargo into human cells and show the effects of the loaded vesicles on target cells (proliferation and cell invasion of human fibroblasts). Examples 9-11 demonstrate the loading of CD24 cargo into liposomes, and the arrangement of multiple copies of the cargo onto both the surface and within individual liposomes.

### Vesicle Loading

The cargo molecule comprising CD24 or a biologically active fragment or variant thereof that is selected for vesicle loading may be coupled with a CPP by covalent or non-covalent binding. In some embodiments, non-covalent complexes between cargos and CPPs are formed. For example, a CPP called Pep-1 can non-covalently bind to a cargo and the resulting binding complex may be loaded into EVs, LVs, or other vesicles (M. C. Morris, J. Depollier, J. Mery, F. Heitz, and G. Divita “A peptide carrier for the delivery of biologically active proteins into mammalian cells”, nature biotechnology, 2001, 19, 1173-1176). A CPP called Candy can non-covalently bind to a nucleic acid cargo and the resulting binding complex may be loaded into EVs, LVs, or other vesicles (L. Crombez, et al., “A New Potent Secondary Amphipathic Cell-penetrating Peptide for siRNA Delivery Into Mammalian Cells”, Mol. Ther. 17, 95-103). An artificial protein called B1 can non-covalently bind to RNA or DNA and the resulting binding complex may be loaded into EVs, LVs, or other vesicles (R.L. Simeon, A. M. Chamoun, T. McMillin, and Z. Chen, “Discovery and Characterization of a New Cell-Penetrating Protein”, ACS. Chem. Biol., 2013, 8, 2678-2687). An engineered superpositively charged GFP called +36 GFP can non-covalently bind to RNA or DNA and the resulting binding complex may be loaded into EVs, LVs, or other vesicles (B. R. McNaughton, J. J. Cronican, D. B. Thompson, and D. R. Liu, “Mammalian cell penetration, siRNA transfection, and DNA transfection by supercharged proteins”, PNAS, 2009, 106, 6111-6116)).

The cargo molecule comprising CD24 or a biologically active fragment or variant thereof that is selected for vesicle loading may be chemically conjugated to a CPP by a disulfide bond, an amide bond, a chemical bond formed between a sulfhydryl group and a maleimide group, a chemical bond formed between a primary amine group and an N-Hydroxysuccinimide (NETS) ester, a chemical bond formed via Click chemistry, or other covalent linkage. “Click” chemistry reactions are a class of reactions commonly used in bio-conjugation, allowing the joining of selected substrates with specific biomolecules. Click chemistry is not a single specific reaction, but describes a method of generating products that follow examples in nature, which also generates substances by joining small modular units. Click chemistry is not limited to biological conditions: the concept of a “click” reaction has been used in pharmacological and various biomimetic applications; however, these reactions have proven useful in the detection, localization, and qualification of biomolecules (H. C. Kolb; M. G. Finn; K. B. Sharpless, “Click Chemistry: Diverse Chemical Function from a Few Good Reactions”, Angewandte Chemie International Edition, 2001, 40 (11):2004-2021; and R. A. Evans, “The Rise of Azide-Alkyne 1,3-Dipolar ‘Click’ Cycloaddition and its Application to Polymer Science and Surface Modification”, Australian Journal of Chemistry, 2007, 60 (6): 384-395).

Optionally, the cargo molecule is covalently coupled to the CPP by a cleavable domain or linker, which becomes cleaved upon exposure of the binding complex to the appropriate cleaving agent or condition, such as a chemical agent (e.g., dithiothreitol for reducing a disulfide bond linkage), environment (e.g., temperature or pH), or radiation. For example, the cleavable domain or linker may be photo-cleavable (Olejnik, J. et al., “Photocleavable peptide-DNA conjugates: synthesis and applications to DNA analysis using MALDI-MS”, Nucleic Acids Research, 1999, 27 (23):4626-4631; Matsumoto Ret al., “Effects of the properties of short peptides conjugated with cell-penetrating peptides on their internalization into cells,” Scientific Reports, 2015, 5:12884; and Usui, K. et al., “A novel array format for monitoring cellular uptake using a photo-cleavable linker for peptide release”, Chem Commun, 2013, 49:6394-6396; Kakiyama, T. et al., “A peptide release system using a photo-cleavable linker in a cell array format for cell-toxicity analysis”, Polymer J., 2013, 45:535-539; Wouters, S. F. A., Wijker, E., and Merkx, M., “Optical Control of Antibody Activity by Using Photocleavable Bivalent Peptide-DNA Locks”, ChemBioChem, 2019, 20:2463-2466). By linking the cargo molecule with a CPP via a photo-cleavable conjugation, once the binding complex is inside an EV, LV, or other vesicle, the vesicle can be exposed to light of the proper wavelength, which will cleave the linker between the CPP and the cargo molecule, freeing the cargo inside the vesicle. Once the vesicle fuses with a cell, the free cargo will be delivered into the cell.

In embodiments in which the cargo molecule is a nucleic acid (such as a nucleic acid encoding a full-length CD24 polypeptide or a biologically active fragment thereof), fusion with the CPP may be achieved through a chemical bond.

Likewise, in embodiments in which the cargo molecule is a nucleic acid, tight association with the CPP may be achieved through non-covalent binding.

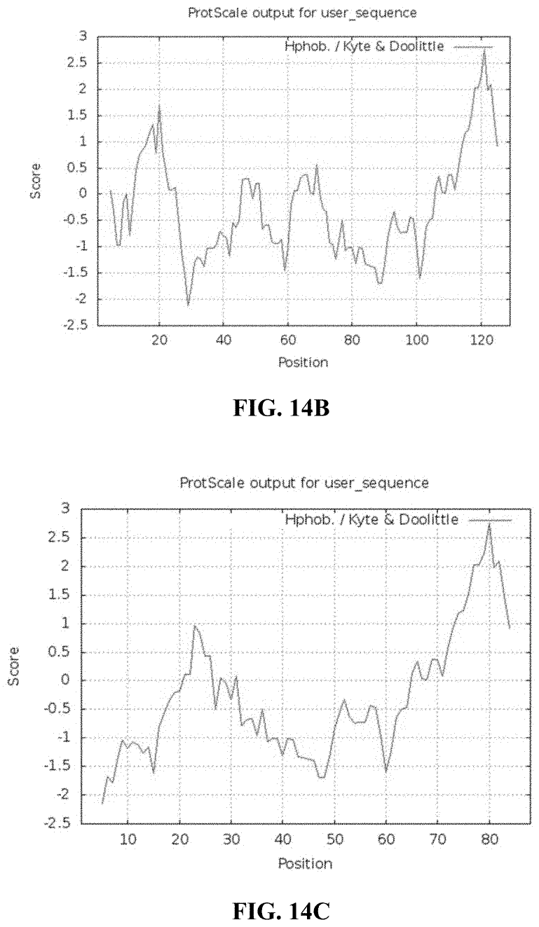

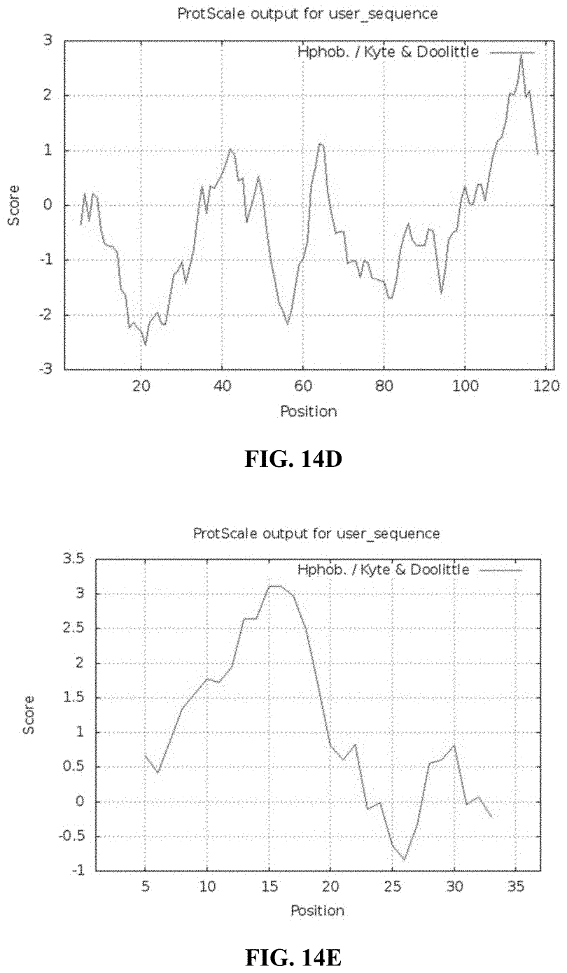

Transmembrane domains function as membrane anchors. Based on its amino acid sequence, the inventors predicted that human CD24 contains several hydrophobic residues that are close to its C-terminus and are predicted to form one hydrophobic alpha-helix for anchoring onto the vesicle membrane or outer layer (see sequence alignment of FIG. 13 and hydropathy plots of FIGS. 14A-14E). In some embodiments, the CD24 molecule loaded on the vesicle includes one or more transmembrane regions comprising the transmembrane domain shown in FIG. 13 (amino acid sequence ALQSTASLFVVSLSLLHLYS). Advantageously, by including one or more transmembrane regions, extracellular regions of the CD24 molecule can be correctly displayed on the vesicle's outer surface.

Optionally, a cysteine residue can be added to the C-terminus of the cargo molecule to attach a detectable agent or medical imaging agent, such as a fluorescent compound (e.g., a fluorophore) to serve as a marker, dye, tag, or reporter, as is described in the Examples.

The following are specific examples of covalent linkages. Human or animal CD24 molecules (CD24, or a biologically active fragment of CD24, or biologically active variant of CD24) can be covalently coupled to a CPP via one of the following ways before being incubated and loaded to purified vesicles: (1) A chosen CPP can be genetically fused to the C-terminus of CD24 or fragment or variant thereof to encode the fusion protein (CD24-CPP) in an expression plasmid. CD24-CPP can be expressed in host cells such as E. coli, yeast, plants, insect cells, or mammalian cells before being purified via column chromatography. The purified fusion protein CD24-CPP can be incubated with vesicles and then enter and be bound by the outer layer of vesicles with the extracellular residues of CD24 or fragment or variant displayed on the outside surface of the vesicles. Some molecules of CD24-CPP will also enter the lumen side of the vesicles. (2) If the covalent linkage between CD24 or fragment or variant and a CPP is a disulfide bond, the CD24 will be engineered to contain an extra C-terminal cysteine residue (CD24-Cys) in an expression plasmid while the synthetic CPP peptide contains an N-terminal cysteine residue (Cys-CPP). The CD24-Cys can be expressed and purified similarly as the above-mentioned CD24-CPP. The Cys-CPP can be chemically synthesized and purified via high performance liquid chromatography (HPLC). After oxidization, the purified CD24-Cys and Cys-CPP will form a disulfide bond and yield CD24-Cys-Cys-CPP. After purification, CD24-Cys-Cys-CPP can be incubated with vesicles for loading. Like CD24-CPP, many loaded molecules of CD24-Cys-Cys-CPP will be bound on the outer membrane of the loaded vesicles while others will exist in the lumen side of the vesicles. If needed, the loaded vesicles can be subsequently incubated with a reducing agent, e.g. dithiothreitol (DTT), to break the disulfide linkage and free the CD24-Cys from Cys-CPP. (3) If the covalent coupling between CD24 or the biologically active fragment or variant and a CPP is via a photo-cleavable bond (CD24-clevable bond-CPP), both the C-terminus of the CD24 or fragment or variant and the N-terminus of the CPP can be chemically engineered. As in (2), after loading to vesicles, the photo-cleavable bond can be broken by exposing the CD24-clevable bond-CPP containing vesicles with appropriate wavelength lights. Once the photo-cleavable bond is broken, the CD24 or fragment or variant thereof will be free of the CPP and is either bound by the vesicle membrane or exists within the lumen side of the vesicles.

The loading method may include the step of covalently or non-covalently coupling the CPP to the cargo molecule, to produce the binding complex, before contacting the vesicle with the binding complex.

In other aspect, the CPP and cargo molecule to be uncoupled (physically separated) within the vesicles if the CPP interferes with the in vivo function of the cargo, or the binding complex causes additional side effect(s) in vivo relative to the cargo itself (if there are such side effects). In one aspect, the loading method may also include the step of uncoupling the CPP and the CD24 cargo molecule once the cargo molecule has been internalized by the vesicle, associated with the vesicle, or a combination thereof. Once the CD24 cargo is loaded into vesicles, it is not necessary to have the binding complex stay intact as long as the cargo molecules are either inside the vesicles or embedded onto the membrane of the vesicles, depending on the intended use of the loaded vesicles. If the CPP is non-covalently coupled to the CD24 cargo molecule, the complex can either associate or dissociate within the vesicles. If the CPP is covalently coupled to the CD24 cargo molecule, the complex may be intact or be intentionally cleaved, for example by light, a reducing agent such as dithiothreitol (DTT) or other methods.

In other aspects, it may not be necessary to uncouple the CPP and cargo molecule of the binding complex if the CPP does not interfere with the in vivo function of the cargo molecule and the binding complex has the same side effect profile as the cargo molecule alone (if there are such side effects).

Another aspect of the invention is the loaded vesicle itself, which includes a cargo molecule comprising CD24, or biologically active fragment or variant thereof, coupled to a CPP covalently or non-covalently (together, the “binding complex”), wherein the binding complex has been internalized by the vesicle, associated with the vesicle, or a combination thereof. The loaded vesicle may be produced using any of the aforementioned embodiments of methods for loading the EV. Thus, the linkage between the CPP and cargo molecule may be covalent or non-covalent.

In addition to a CD24 molecule, the cargo of the loaded vesicle may include additional molecules such as a small molecule, fluorescent dye, imaging agent, macromolecule, polypeptide (natural or modified), nucleic acid (e.g., DNA, RNA, PNA, DNA- or RNA-like molecule, snRNA, ncRNA (e.g., miRNA), RNAi (e.g., siRNA, shRNA) mRNA, tRNA, antibody or antibody-fragment, proteins (e.g., enzymes, membrane-bound proteins), growth factor, lipoprotein, protein, carbohydrate, or glycoprotein. The additional molecule may be any class of substance or combination of classes. The additional molecule may be in the form of an active pharmaceutical ingredient or a pharmaceutically acceptable salt, metabolite, derivative, or prodrug of an active pharmaceutical ingredient.

### Signal Transducer CD24 as Cargo Molecule

The payload to be delivered to cells in vitro or in vivo is referred to herein as the “cargo” or a “cargo molecule” and includes, at least, signal transducer CD24 (also known as cluster of differentiation 24, or heat stable antigen CD24 (HSA)), or a biologically active fragment or variant thereof. CD24 is a small glycosyl-phosphatidyl-inositol (GPI)-anchored glycoprotein with widespread expression among both hematopoietic and non-hematopoietic cells, which is encoded by a coding sequence of 240 base pairs. Of the 80 amino acids, the first 26 amino acids constitute the signal peptide, while the last 23 amino acids serve as a signal for cleavage to allow for the attachment of the GPI tail. As a result, the mature human CD24 molecule has only 31 amino acids (International Application Publication WO 2017/136492; Liu Y et al.; published Aug. 10, 2017). Of the 31 amino acids in the mature CD24 protein, the last (C-terminal) amino acid is polymorphic among the human population. A C-to-T transition at nucleotide 226 results in the substitution of alanine (a) with valine (v). Since this residue is immediately N-terminal to the cleavage site, and since the replacement is non-conservative, these two alleles may be expressed at different efficiencies on the cell surface.

The full-length 80-amino acid sequence of human signal transducer CD24 (Uniprot accession number P25063) is the following: MGRAMVARLGLGLLLLALLLPTQIYS [symbol] [symbol] [symbol] [symbol] GGALQSTASLFVVSLSLLHLYS. The underlined portion (amino acids 1-26) is the N-terminal signal peptide. The bold italicized portion is the mature peptide, which is 33 amino acids in length (amino acids 27-59). The remaining portion of the sequence is the pro-peptide (amino acids 60-80), which is the portion of the protein that is cleaved during maturation or activation (processing). FIG. 13 herein is a sequence alignment of the isoforms of human signal transducer CD24. This amino acid sequence comparison shows that all CD24 isoforms evaluated have an identical transmembrane region with the exception of isoform A0A087WU21. Hydropathy plots are shown in FIGS. 14A-14E.

The CD24 loaded on the vesicle may include the amino acid sequence of the mature polypeptide SETTTGTSSNSSQSTSNSGLAPNPTNATTKAAG, or a biologically active fragment or variant thereof, and optionally may include a signal sequence, such as MGRAMVARLGLGLLLLALLLPTQIYS.

The CD24 loaded on the vesicle may include the amino acid sequence of the extracellular domain (ECD) of the human CD24: SETTTGTSSNSSQSTSNSGLAPNPTNATTK, or a biologically active fragment or variant thereof. As indicated above, using the loading methods of the invention, the extracellular regions of the CD24 molecule can be correctly displayed on the vesicle's outer surface, which is advantageous for biological function.

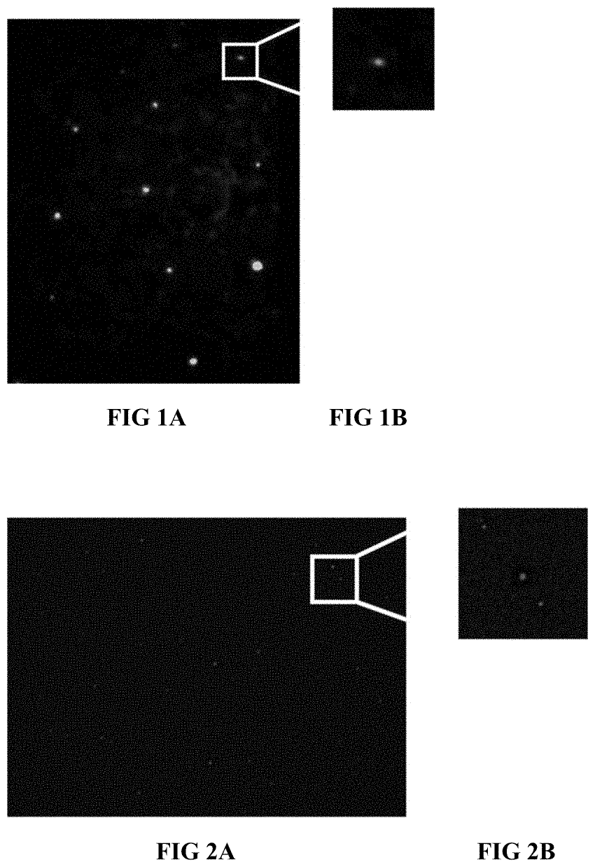

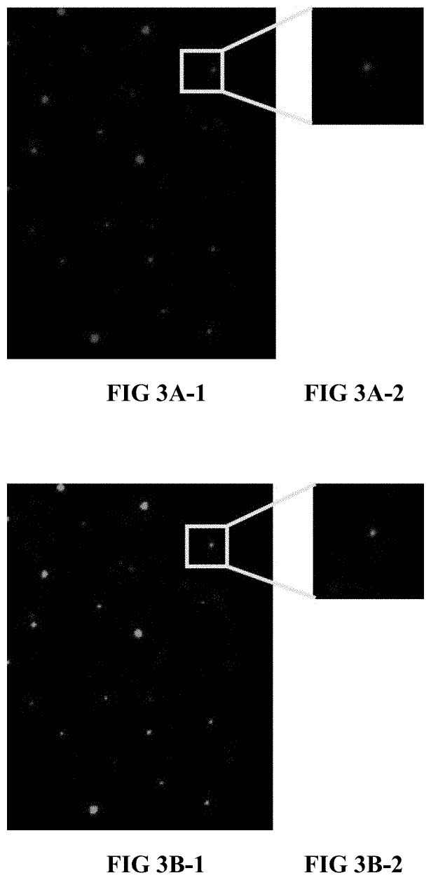

In some embodiments, the CD24 loaded on the vesicle is SETTTGTSSNS SQSTSNSGLAPNPTNATTK, SETTTGTSSNSSQSTSNSGLAPNPTNATTK(V/A), or mouse CD24, which may be NQTSVAPFPGNQNISASPNPTNATTRG, as described in U.S. Patent Application Publication US 2013/0231464 (Zheng et al., “Methods of Use of Soluble CD24 for Therapy of Rheumatoid Arthritis”, published Sep. 5, 2013, which is incorporated herein by reference in its entirety), or a biologically active fragment or variant of any of the foregoing. The CD24 may also have an amino acid sequence described in FIG. 1 or 2 of U.S. Patent Application Publication US 2013/0231464. The CD24 may exist in one of two allelic forms, such that the C-terminal amino acid of the mature human CD24 may be a valine or an alanine. The C-terminal valine or alanine may be immunogenic and may be omitted from the CD24 to reduce its immunogenicity.

As taught in U.S. Patent Application Publication US 2013/0231464, despite considerable sequence variations in the amino acid sequence of the mature CD24 proteins from mouse and human, they are functionally equivalents in interaction with the danger-associated molecular patterns (DAMP). As a result of sequence conservation between mouse and human CD24 primarily in the C-terminus and in the abundance of glycosylation sites, significant variations in the mature CD24 proteins may be tolerated in using the CD24 to treat RA, especially if those variations do not affect the conserved residues in the C-terminus or do not affect the glycosylation sites from either mouse or human CD24.

Optionally, the cargo comprises a fusion protein that includes CD24 or biologically active fragment or variant thereof (a “CD24 fusion protein”) and one or more other molecules or sequences heterologous to the CD24 sequence, In some embodiments, the CD24 or biologically active fragment or variant thereof is fused at its N-terminal end and/or its C-terminal end to a further coding or non-coding sequence. In some embodiments, the CD24 or biologically active fragment thereof is fused at its C-terminal or N-terminal end to an immunoglobulin (Ig) or a portion thereof, preferably a mammalian Ig or portion thereof. The Ig protein may be human IgG₁, IgG₂, IgG₃, IgG₄, IgM, or IgA.

In some embodiments, the Ig portion is an antigen-binding fragment Fab (fragment, antigen binding) region.

In some embodiments, the Ig portion is an Fc (fragment, crystallizable) region. In some embodiments, the Fc region includes the hinge region and CH2 and CH3 domains of the Ig protein. in some embodiments, the Fc region comprises a human IgGi Fc region as set forth in SECS ID NO: 6 of U.S. Patent Application Publication US 2013/0231464, wherein the human IgG₁ Fc region is fused to the N-terminus or C-terminus of the mature human CD24. In some embodiments, the Ig protein is an IgM, and the Fc portion includes the hinge region and. CH3 and CH4 domains of IgM.

The CD24 may also be fused at its N- or C-terminus to a protein tag, which may be GST (the glutathione S-transferase tag), His (the polyhistidine tag), or FLAG (a peptide protein tag with the sequence of DYKDDDDK). Methods for making fusion proteins and purifying fusion proteins are well known in the art.

In some embodiments, the CD24 fusion protein is CD24IgG₁Fc (also referred to as CD24Fc) shown in FIG. 1A of U.S. Patent Application Publication US 2013/0231464. The amino acid composition of CD24IgGiFc is also shown in “A” below. The underlined 26 amino acids are the signal peptide of CD24. The boxed, bold portion of the sequence is the mature CD24 protein used in the fusion protein. The last amino acid (A or V) that is ordinarily present in the mature CD24 protein has been deleted from the construct to reduce or avoid immunogenicity. The non-underlined, non-bold letters are the sequence of IgG1 Fc, including the hinge region and CH1 and CH2 domains. In some embodiments, the CD24 fusion protein is CD24^{V}Fc in FIG. 1B of U.S. Patent Application Publication US 2013/0231464, in which the mature human CD24 protein is the valine polymorphic variant. The amino acid composition of CD24^{V}Fc is shown in “B” below, and the various parts of the fusion protein are marked as in “A”.

A.

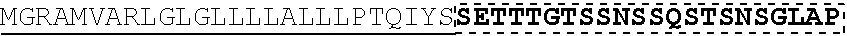

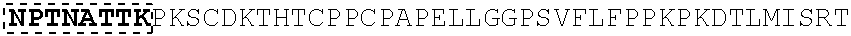

PEVTCVVVDVSHEDPEVKFNWYVDGVEVHNAKTKPREEQYNSTYRVVS
VLTVLHQDWLNGKEYKCKVSNKALPAPIEKTISKAKGQPREPQVYTLP
PSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGK
B.

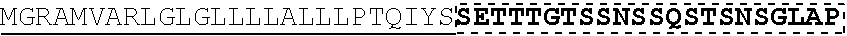

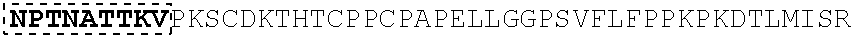

TPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTL
PPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK

Amino acid sequence variations between mature CD24 proteins from mouse and human are shown below, with the potential glycosylation sites in bold.

Mouse 24 NQTSVAPFPGN--QNISAS----PNPTNATTRG
-* - * * * * ********--
Human CD24 SETTTGTSS-NSSQSTSNS-GLAPNPTNATTKA(V)

The CD24 or biologically active fragment or variant thereof may be prepared using a eukaryotic expression system. The expression system may involve expression from a vector in mammalian cells, such as Chinese Hamster Ovary (CHO) cells. The system may also be a viral vector, such as a replication-defective retroviral vector that may be used to infect eukaryotic cells. The CD24 or biologically active fragment or variant thereof may also be produced from a stable cell line that expresses CD24 or biologically active fragment or variant thereof from a vector or a portion of a vector that has been integrated into the cellular genome. The stable cell line may express CD24 or a biologically active fragment or variant from an integrated replication-defective retroviral vector. The expression system may be GPEx™, for example.

The cargo molecule comprising CD24 or biologically active fragment or variant thereof can be covalently or non-covalently coupled with a natural, modified, or artificial CPP at its N- or C-terminus. In the case of covalent coupling, the cargo molecule can be coupled to a CPP via either a disulfide bond, an amide bond, a chemical bond formed between a sulfhydryl group and a maleimide group, a chemical bond formed between a primary amine group and an N-Hydroxysuccinimide (NHS) ester, a chemical bond formed via Click chemistry, or other covalent linkages. The coupled cargo is denoted as “the binding complex”. Following are several scenarios: i) if the cargo is a polypeptide with a small to medium size, such as a CD24 polypeptide or an additional peptide to be included with the CD24 polypeptide, the binding complex can be chemically synthesized; ii) if the binding complex is a CPP fused to either the N-terminus or C-terminus of a large sized polypeptide such as a protein (or inserted into any chosen site of the protein), the encoding DNA sequence of the binding complex can be inserted into an eukaryotic organism, e.g. plants, or insect or mammalian cells, for expression and purification; iii) if the cargo contains a nucleic acid, such as a nucleic acid encoding a CD24 polypeptide or an additional nucleic acid, the cargo can be chemically synthesized, made by reverse or regular polymerase chain reaction (PCR), made by ligation from smaller pieces of nucleic acids, or by other means. The nucleic acid will then be purified by high performance liquid chromatography (HPLC) or other means. The purified nucleic acid can then be covalently or non-covalently coupled to a CPP to form the binding complex; and iv) if the cargo contains a lipid, a metabolite, a small or large chemical molecule, a dye, a sugar, a medical imaging agent, or a small molecule drug, in combination with the CD24 molecule, the cargo can be chemically synthesized and HPLC purified. The purified cargo can then be coupled to a CPP via either disulfide, an amide bond, a chemical bond formed between a sulfhydryl group and a maleimide group, a chemical bond formed between a primary amine group and an N-Hydroxysuccinimide (NETS) ester, a chemical bond formed via Click chemistry, or other covalent linkages to form the binding complex.

Second, the binding complex can be purified via column chromatography, HPLC, or other means. Third, the purified binding complex can be incubated with and then enter the vesicle. These are referred to as a “loaded vesicle” (e.g., “loaded EV” or “loaded LV”). Fourth, the linkages of certain covalent conjugation, e.g. the disulfide linkage, can be broken by incubating the loaded vesicles with small lipid bilayer-penetrating molecules, e.g. dithiothreitol (DTT) for reducing the disulfide linkage, leading to the formation of cargos free of the CPP inside the loaded vesicle. Alternatively, once the loaded vesicles fuse with host cells and the CPP-cargo conjugated via a disulfide linkage enter the cells, the disulfide linkage will be broken by a cellular reducing environment, freeing the cargo inside the cells. If the cargo molecule is covalently linked with a CPP via photo-cleavable conjugation, the binding complex inside a vesicle can be cleaved into the CPP and the cargo molecule once the vesicle is exposed to light of the proper wavelength. This will free the cargo inside the vesicle. Finally, the loaded vesicles will be administered to cells in vitro or an organism in vivo, e.g. a human or non-human animal subject, and then fuse with various cells of the organism for cargo delivery. Once inside the organism's cells, the cargo molecules comprising CD24 or biologically active fragments or variants (and optionally additional molecules) can play various biological roles and affect the function and behavior of the organism's cells, relevant tissues, organs, and/or even the entire organism.

In some embodiments, the CPP can be inserted in a position of any loop regions which do not have secondary structure and do not interact with other parts of the polypeptide cargo.

The cargo may further include one or more molecules in combination with the CD24 or biologically active fragment or variant thereof. The other molecules may be directly or indirectly fused to the CD24 or biologically active fragment thereof or may be included unfused.

These further molecules, which may be included as cargo in combination with the CD24 molecule, may belong to any class of substance or combination of classes. Examples of further cargo molecules include, but are not limited to, a small molecule (e.g., a drug), macromolecule such as polyimides, proteins (e.g., enzymes, membrane-bound proteins), polypeptide (natural or modified), nucleic acid (e.g., natural, damaged or chemically modified DNA, DNA plasmid or vector, telomere, DNA quadruplex, DNAzyme, DNA-like molecule, antisense oligonucleotide, locked nucleic acid, threose nucleic acid, peptide nucleic acid (PNA), single or double-stranded nucleic acid, natural, damaged or chemically modified RNA, catalytic RNA, RNAzyme, ribozyme, non-coding RNA (ncRNA) such as miRNA, snRNA, interfering RNA such siRNA or shRNA, single guide RNA for Cas9, and mRNA, tRNA, and ribosomal RNA (rRNA)), antibody or antibody-fragment, lipoprotein, carbohydrate, or glycoprotein. In some embodiments, the further molecule is a hormone, metabolite, signal molecule, vitamin, or anti-aging agent.

In some embodiments, in addition to a CD24 molecule, the cargo may further include DNA, which may be inhibitory, such as an antisense oligonucleotide, or the DNA may encode a polypeptide and can optionally include a promoter operably linked to the encoding DNA for expression in cells in vitro or in vivo. In some embodiments, the cargo molecule is an RNA molecule such as snRNA, ncRNA (e.g. miRNA), mRNA, tRNA, catalytic RNA, RNAzyme, ribozyme, interfering RNA (e.g., shRNA, siRNA), or guide RNA (e.g., sgRNA) for gene editing by a gene editing enzyme (e.g., Cas9).

Optionally, small RNAs (tRNAs, Y RNAs, sn/sno RNAs) can be glycosylated (called “glycoRNAs”) and anchored to the membrane or outer layer of the vesicles. Small noncoding RNAs bearing sialylated glycans have been found on the cell surface of multiple cell types and mammalian species, in cultured cells, and in vivo, and were determined to interact with anti-dsRNA antibodies and members of the Siglec receptor family (Flynn RA et al., “Small RNAs are modified with N-glycans and displayed on the surface of living cells”, Cell 2021, 184:3109-3124). GlycoRNAs can be included with the CD24 as part of the cargo molecule, which is coupled to the CPP to form a binding complex and loaded onto the vesicle. Alternatively, glycoRNA may be a separate cargo molecule, coupled to a CPP to form another binding complex, which is loaded onto the vesicle. In either case, the glycoRNA can be loaded onto the vesicle for display on the outer lipid layer of the vesicle.

In some embodiments, in addition to a CD24 polypeptide, the cargo further includes a monoclonal or polyclonal antibody, or a portion thereof, such as a constant region (Fc region) or antigen-binding fragment (Fab region) thereof. The antibody or antibody fragment may be a human antibody or fragment, animal antibody fragment, chimeric antibody or fragment, or humanized antibody or fragment.

In those embodiments in which the cargo includes both a CD24 molecule and an antibody or antibody fragment, the CPP may be fused to the CD24 molecule or the antibody or antibody fragment. For example, the CPP may be coupled at the C-termini of the heavy chains of the antibody or antibody fragment, as opposed to the N-termini of the heavy or light chains (as shown by FIG. 2B of Zhang J-F et al., “A cell-penetrating whole molecule antibody targeting intracellular HBx suppresses hepatitis B virus via TRIM21-dependent pathway”, Theranostics, 2018, 8 (2):549-562). Fusion of the CPP may also be done at a position before or after the hinge (as described in the Abstract and FIG. 1 of Gaston J et al., “Intracellular delivery of therapeutic antibodies into specific cells using antibody-peptide fusions”, Scientific Reports, 2019, 9:18688). Preferably, the CPP is fused at the C-termini of the heavy chains or around the hinges although other fusions sites may be used. For other polypeptide cargos (i.e., polypeptides other than antibodies or antibody fragments), fusion may be done at the N-terminus or C-terminus, or internal loop areas of the polypeptide cargo molecule. Interference with the CD24 and additional cargo molecule's function(s) should be avoided.

In some embodiments, the CD24 or additional cargo molecule, has coupled to it a detectable agent such as a fluorescent (e.g., a fluorophore), luminescent (e.g., a luminophore, Quantum dots), radioactive (e.g., ¹³¹I-Sodium iodide, ¹⁸F-Sodium fluoride) compound to serve as a marker, dye, tag, reporter, medical imaging agent, or contrast agent. Examples of fluorescent proteins that may be coupled to CD24 or an additional cargo molecule include green fluorescent protein (GFP) and GFP-like proteins (Stepanenko OV et al., “Fluorescent Proteins as Biomarkers and Biosensors: Throwing Color Lights on Molecular and Cellular Processes”, Curr Protein Pept Sci, 2008, 9 (4):338-369, which is incorporated herein by reference in its entirety”). In some embodiments, the detectable agent is a quantum dot or other fluorescent probe that may be used, for example, as a contrast agent with an imaging modality such as magnetic resonance imaging (MM). The detectable agent may be coupled to any class of cargo molecule, such as a polypeptide or nucleic acid (e.g., DNA or RNA), to detect, track the location of, and/or quantify the molecule to which it is coupled.

The cargo molecule may be covalently conjugated to the CPP by a disulfide bond, Click chemistry, other covalent linkage, or be non-covalently bound to the CPP.

Optionally, the binding complex includes two or more cargo molecules, which may be the same class of molecule (e.g., two or more polypeptides) or molecules of a different class (e.g., a polypeptide and a small molecule), provided that at least one of the cargo molecules is a CD24 molecule. In some embodiments, the CD24 molecule is a full-length or mature CD24 polypeptide.

### Extracellular Vesicles (EVs)

In some embodiments, the vesicles used in the invention are EVs. EVs are cell-derived and have an interior core surrounded and enclosed by a membrane, with the membrane comprising at least one lipid bilayer. Some EVs have multiple layers of membranes. Examples of EVs, and methods for their isolation and analysis, are described in Antimisiaris SG et al., “Exosomes and Exosome-Inspired Vesicles for Targeted Drug Delivery”, Pharmaceutics, 2018, 10 (4):218; and Doyle L M and M Z Wang, “Overview of Extracellular Vesicles, Their Origin, Composition, Purpose, and Methods for Exosome Isolation and Analysis”, Cells, 2019, 8 (7):727; and Thery C et al., “Minimal information for studies of extracellular vesicles 2018 (MISEV2018): a position statement of the International Society for Extracellular Vesicles and update of the MISEV2014 guidelines”, J. Extracell. Vesicles., 2018, 7:1535750, which are each incorporated herein by reference in their entireties). Any type or subtype of EV may be utilized.

For example, the EV may be an exosome (or small EV), mitovesicle, apoptotic body, microvesicle, microparticle, ectosome, oncosome, or an EV identified by another name in the literature. Depending on the CPP and cargo molecule, upon loading the EV, the binding complex is internalized and contained in the interior of the EV, or is bound and/or embedded within the EV's membrane. In some embodiments, the EV is obtained from a mammalian cell, such as a human cell. In other embodiments, the EV is obtained from a bacterial cell, fungal cell, non-human animal cell, or plant cell.

The EVs may be any shape but are typically spherical, and can range in size from around 20-30 nanometers (nm) to as large as 10 micrometers (μm) or more. Exosomes are typically about 30 nanometers to 150 nanometers in diameter (Doyle L M et al., “Overview of Extracellular Vesicles, Their Origin, Composition, Purpose, and Methods for Exosome Isolation and Analysis” Cells, 2019, 8 (7): 727).

Mammalian cells secrete EVs, which are found in abundant amounts in bodily fluids including blood, saliva, urine, and breast milk. EV particles cannot replicate, and possess a lipid bilayer that separates the EVs' interior (or core) from the outside environment. EVs range in diameter from near the size of the smallest physically possible unilamellar vesicle (around 20-30 nm) to as large as 10 μm or more, although the vast majority of EVs are smaller than 200 nm. For example, exosomes are one type of EVs with a diameter of 30-200 nm. EVs carry a cargo of proteins, nucleic acids, lipids, metabolites, and even organelles from the parent cell. Other than mammalian cells, some bacterial, fungal, and plant cells that are surrounded by cell walls are found to release EVs as well. A wide variety of EV subtypes have been proposed, defined variously by size, biogenesis pathway, cargo, cellular source, and function, leading to a historically heterogeneous nomenclature including terms like exosomes, ectosomes, mitovesicles, microvesicles, microparticles, oncosomes, and apoptotic bodies. Mitovesicles are double-membraned EVs obtained from mitochondria (D′Acunzo et al., “Mitovesicles are a novel population of extracellular vesicles of mitochondrial origin altered in Down syndrome”, Sci. Adv. 2021; 7: eabe5085).

EVs transport various molecules including proteins (e.g., enzymes), metabolites, pro-inflammatory mediators, and nucleic acids (e.g., microRNAs) to other cells and instigate cell regulation and modulation of the immune response in cell-to-cell communication through the EV contents. Although EVs have recently emerged as therapeutic carriers, the major limitation of using EVs has been the lack of a well-developed methodology for increasing cellular uptake of the intended content(s) of EVs.

In some embodiments, the EVs are obtained from a cell that is the same cell type as the target cell or cells for delivery of the cargo molecule(s). In other embodiments, the EVs are derived from a cell that is a different cell type from the cell or cells targeted for delivery. Table 1 below is a non-limiting list of cells from which EVs can be obtained, as well as a non-limiting list of cells to which cargo molecules can be delivered using vesicles (e.g., EVs or LVs) of the invention.

TABLE 1
Examples of Cells

Keratinizing Epithelial Cells
keratinocyte of epidermis
basal cell of epidermis
keratinocyte of fingernails and toenails
basal cell of nail bed
hair shaft cells
medullary
cortical
cuticular
hair-root sheath cells
cuticular
of Huxley's layer
of Henle's layer
external
hair matrix cell
Cells of Wet Stratified Barrier Epithelia
surface epithelial cell of stratified squamous epithelium of cornea tongue, oral cavity,
esophagus, anal canal, distal urethra, vagina
basal cell of these epithelia
cell of urinary epithelium
Epithelial Cells Specialized for Exocrine Secretion
cells of salivary gland
mucous cell
serous cell
cell of von Ebner's gland in tongue
cell of mammary gland, secreting milk
cell of lacrimal gland, secreting tears
cell of ceruminous gland of ear, secreting wax
cell of eccrine sweat gland, secreting glycoproteins
cell of eccrine sweat gland, secreting small molecules
cell of apocrine sweat gland
cell of gland of Moll in eyelid
cell of sebaceous gland, secreting lipid-rich sebum
cell of Bowman's gland in nose
cell of Brunner's gland in duodenum, secreting alkaline solution of mucus and enzymes
cell of seminal vesicle, secreting components of seminal fluid, including fructose
cell of prostate gland, secreting other components of seminal fluid
cell of bulbourethral gland, secreting mucus
cell of Bartholin's gland, secreting vaginal lubricant
cell of gland of Littré, secreting mucus
cell of endometrium of uterus, secreting mainly carbohydrates
isolated goblet cell of respiratory and digestive tracts, secreting mucus
mucous cell of lining of stomach
zymogenic cell of gastric gland, secreting pepsinogen
oxyntic cell of gastric gland, secreting HCl
acinar cell of pancreas, secreting digestive enzymes and bicarbonate
Paneth cell of small intestine, secreting lysozyme
type II pneumocyte of lung, secreting surfactant
Clara cell of lung
Cells Specialized for Secretion of Hormones
cells of anterior pituitary, secreting
growth hormone
follicle-stimulating hormone
luteinizing hormone
prolactin
adrenocorticotropic hormone
thyroid-stimulating hormone
cell of intermediate pituitary, secreting melanocyte-stimulating hormone
cells of posterior pituitary, secreting
oxytocin
vasopressin
cells of gut and respiratory tract, secreting
serotonin
endorphin
somatostatin
gastrin
secretin
cholecystokinin
insulin
glucagons
bombesin
cells of thyroid gland, secreting
thyroid hormone
calcitonin
cells of parathyroid gland, secreting
parathyroid hormone
oxyphil cell
cells of adrenal gland, secreting
epinephrine
norepinephrine
steroid hormones
mineralocorticoids
glucocorticoids
cells of gonads, secreting
testosterone
estrogen
progesterone
cells of juxtaglomerular apparatus of kidney
juxtaglomerular cell
macula densa cell
peripolar cell
mesangial cell
Epithelial Absorptive Cells in Gut, Exocrine Glands, and Urogenital Tract
brush border cell of intestine
striated duct cell of exocrine glands
gall bladder epithelial cell
brush border cell of proximal tubule of kidney
distal tubule cell of kidney
nonciliated cell of ductulus efferens
epididymal principal cell
epididymal basal cell
Cells Specialized for Metabolism and Storage
Hepatocyte
fat cells (e.g., adipocyte)
white fat
brown fat
lipocyte of liver
Epithelial Cells Serving Primarily a Barrier Function, Lining the Lung, Gut,
Exocrine Glands, and Urogenital Tract
type I pneumocyte
pancreatic duct cell
nonstriated duct cell of sweat gland, salivary gland, mammary gland, etc.
parietal cell of kidney glomerulus
podocyte of kidney glomerulus
cell of thin segment of loop of Henle
collecting duct cell
duct cell of seminal vesicle, prostate gland, etc.
Epithelial Cells Lining Closed Internal Body Cavities
vascular endothelial cells of blood vessels and lymphatics (e.g., microvascular cell)
fenestrated
continuous
splenic
synovial cell
serosal cell
squamous cell lining perilymphatic space of ear
cells lining endolymphatic space of ear
squamous cell
columnar cells of endolymphatic sac
with microvilli
without microvilli
“dark” cell
vestibular membrane cell
stria vascularis basal cell
stria vascularis marginal cell
cell of Claudius
cell of Boettcher
choroid plexus cell
squamous cell of pia-arachnoid
cells of ciliary epithelium of eye
pigmented
nonpigmented
corneal “endothelial” cell
Ciliated Cells with Propulsive Function
of respiratory tract
of oviduct and of endometrium of uterus
of rete testis and ductulus efferens
of central nervous system
Cells Specialized for Secretion of Extracellular Matrix
epithelial:
ameloblast
planum semilunatum cell of vestibular apparatus of ear
interdental cell of organ of Corti
nonepithelial:
fibroblasts
pericyte of blood capillary (Rouget cell)
nucleus pulposus cell of intervertebral disc
cementoblast/cementocyte
odontoblast/odontocyte
chondrocytes
of hyaline cartilage
of fibrocartilage
of elastic cartilage
osteoblast/osteocyte
osteoprogenitor cell
hyalocyte of vitreous body of eye
stellate cell of perilymphatic space of ear
Contractile Cells
skeletal muscle cells
red
white
intermediate
muscle spindle-nuclear bag
muscle spindle-nuclear chain
satellite cell
heart muscle cells
ordinary
nodal
Purkinje fiber
Cardiac valve tissue
smooth muscle cells
myoepithelial cells:
of iris
of exocrine glands
Cells of Blood and Immune System
red blood cell (erythrocyte)
Megakaryocyte
Macrophages
monocyte
connective tissue macrophage
Langerhan's cell
osteoclast
dendritic cell
microglial cell
Neutrophil
Eosinophil
Basophil
mast cell
plasma cell
T lymphocyte
helper T cell
suppressor T cell
killer T cell
B lymphocyte
IgM
IgG
IgA
IgE
killer cell
stem cells and committed progenitors for the blood and immune system
Sensory Transducers
Photoreceptors
rod
cones
blue sensitive
green sensitive
red sensitive
Hearing
inner hair cell of organ of Corti
outer hair cell of organ of Corti
acceleration and gravity
type I hair cell of vestibular apparatus of ear
type II hair cell of vestibular apparatus of ear
Taste
type II taste bud cell
Smell
olfactory neuron
basal cell of olfactory epithelium
blood pH
carotid body cell
type I
type II
Touch
Merkel cell of epidermis
primary sensory neurons specialized for touch
Temperature
primary sensory neurons specialized for temperature
cold sensitive
heat sensitive
Pain
primary sensory neurons specialized for pain
configurations and forces in musculoskeletal system
proprioceptive primary sensory neurons
Autonomic Neurons
Cholinergic
Adrenergic
Peptidergic
Supporting Cells of Sense Organs and of Peripheral Neurons
supporting cells of organ of Corti
inner pillar cell
outer pillar cell
inner phalangeal cell
outer phalangeal cell
border cell
Hensen cell
supporting cell of vestibular apparatus
supporting cell of taste bud
supporting cell of olfactory epithelium
Schwann cell
satellite cell
enteric glial cell
Neurons and Glial Cells of Central Nervous System
Neurons
glial cells
astrocyte
oligodendrocyte
Lens Cells
anterior lens epithelial cell
lens fiber
Pigment Cells
Melanocyte
retinal pigmented epithelial cell
iris pigment epithelial cell
Germ Cells
oogonium/oocyte
Spermatocyte
Spermatogonium
blast cells
fertilized ovum
Nurse Cells
ovarian follicle cell
Sertoli cell
thymus epithelial cell (e.g., reticular cell)
placental cell

EVs may also be obtained from immature progenitor cells or stem cells. Cells can range in plasticity from totipotent or pluripotent stem cells (e.g., adult or embryonic), precursor or progenitor cells, to highly specialized cells, such as those of the central nervous system (e.g., neurons and glia). Stem cells and progenitor cells can be obtained from a variety of sources, including embryonic tissue, fetal tissue, adult tissue, adipose tissue, umbilical cord blood, peripheral blood, bone marrow, and brain, for example.

As will be understood by one of skill in the art, there are over 200 cell types in the human body. EVs can be obtained from any of these cell types for use in the invention. For example, any cell arising from the ectoderm, mesoderm, or endoderm germ cell layers can be used. Likewise, the cargo molecules comprising CD24 by itself, or in combination with further molecules, can be delivered to any cell or cells by EVs. The recipient cells of the cargo molecules may be of the same cell type from which the EV is obtained, or a different cell type. Recipient cells may be natural or wild-type cells, or cells of a cell line, for example.

In some embodiments, the EV is an exosome derived from a human mesenchymal stem cell (hMSC). Sources of mesenchymal stem cells include adult tissues, such as bone marrow, peripheral blood, and adipose tissue, as well as neonatal birth-associated tissues, such as placenta, umbilical cord, and cord blood. The hMSC-derived EVs have a variety of potential applications.

Optionally, EVs such as exosomes may include a targeting agent that targets the EV to a cell type, organ, or tissue. An EV membrane-bound ligand can be engineered to bind to and fuse with a specific cell type, tissue, or organ and deliver the cargo into the target cells, tissue or organ.

Liver targeting: It has been observed that most exosomes injected into mouse tail vein or intravenous administration into normal mice are distributed into livers. Without being limited by theory of mechanism of action, liver cell-derived EVs loaded with inhibitors or other therapeutic agents via CPPs can be intravenously administered into human or animal subjects for treating various liver diseases, disorders, or conditions, such as hepatitis A/B/C infections, liver cancer, and hepatic steatosis.

EVs are enriched in tetraspanin proteins like CD9, CD63, and CD81 that are common to many cell-derived EVs. Tissue-specific or disease-specific EV markers have been identified, e.g. PCA3 from prostate cancer cells. Dependent upon the cell sources, EVs including exosomes have been found to contain other EV markers including CD37, CD82, and Lamp2b. The following are merely examples of how EVs loaded with cargos via CPPs may be used to target specific cells/organs/tissues.

Nerve or neuronal cell targeting: Phage display is used to select peptide CP05 (CRHSQMTVTSRL) which can bind tightly to exosomal protein CD63, and peptide NP41 (NTQTLAKAPEHT) which can bind to peripheral nerves. Once fused, the peptide NP41-CP05 can bind to CD63 in exosomes and guide the exosomes to target nerves (Gao et al., “Anchor peptide captures, targets, and loads exosomes of diverse origins for diagnostics and therapy”, Sci. Transl. Med. 2018, 10, eaat0195, which is incorporated herein by reference in its entirety). Such engineered EVs can be loaded with cargo molecules coupled with a CPP, and used as therapeutic agents to treat nerve diseases, disorders, and conditions.

Similarly, CP05 is fused with the neuronal cell-specific peptide RVG (YTIWMPENPRPGTPCDIFTNSRGKRASNG) and this fusion peptide can bind to CD63 in exosomes and guide the EV to target neuronal cells (see FIG. 1A of Gao et al., 2018). Such engineered EVs can be loaded with cargos coupled with a CPP, and used as therapeutic agents to treat neural diseases, disorders, and conditions of the central and peripheral nervous systems.

Muscle targeting: Phage display may be used to select peptide M12 (RRQPPRSISSHP) which preferentially binds to skeletal muscle. Thus, the peptide M12-CP05 can bind to CD63 in exosomes and guide exosomes to target muscle (Gao et al., 2018). Such engineered EVs can be loaded with cargos coupled with a CPP and used as therapeutic agents to treat muscle diseases, disorders, and conditions.

Neuronal cell targeting: Exosomal protein Lamp2b is genetically fused to peptide RVG (YTIWMPENPRPGTPCDIFTNSRGKRASNG). The fusion protein RVG-Lamp2b is expressed in the dendritic cells which secrete exosomes containing bound RVG-Lamp2b on their exosomal membrane while RVG is displaced on the membrane surface. The engineered exosomes are loaded with exogenous siRNA by electroporation. Intravenously injected RVG-Lamp2b containing exosomes can deliver GAPDH siRNA specifically to neurons, microglia, oligodendrocytes in the brain, resulting in a specific gene knockdown (Alvarez-Erviti et al., “Delivery of siRNA to the mouse brain by systemic injection of targeted exosomes”, Nat. Biotechnol. 2011; 29: 341-345, which is incorporated herein by reference in its entirety). Such engineered EVs can be loaded with cargos coupled with a CPP and used as therapeutic agents to treat neuronal diseases, disorders, and conditions.

Cancer cell targeting: Exosomal protein Lamp2b is genetically fused to a fragment of Interleukin 3 (IL3). The fusion protein IL3-Lamp2b is expressed in HEK293T cells which secrete exosomes containing bound IL3-Lamp2b on their exosomal membrane while IL3 is displaced on the membrane surface. These IL3-Lamp2b-expressing HEK293T cells are incubated or transfected with an anti-cancer drug such as imatinib, or BCR-ABL siRNA, which secrete loaded IL3-Lamp2b-contianing exosomes. These specially engineered exosomes can bind to the IL3 receptor (IL3-R) overexpressed in chronic myeloid leukemia (CML) blasts, leading to the inhibition of in vitro and in vivo cancer cell growth (Bellavia et al., Interleukin 3-receptor targeted exosomes inhibit in vitro and in vivo Chronic Myelogenous Leukemia cell growth”, Theranostics 2017, 7(5), 1333-1345, which is incorporated herein by reference in its entirety). Such engineered EVs can be loaded with anti-cancer cargos via a CPP and used as therapeutic agents to treat cancer and other cell proliferation disorders.

### Lipid Vesicles (LVs)

In some embodiments, the vesicle used in the invention is an LV. LVs are particles having an interior core surrounded and enclosed by at least one lipid layer. Each of the one or more lipid layers surrounding the core may be a lipid monolayer or a lipid bilayer. Any type of LV may be utilized, such as a liposome, lipid nanoparticle, lipid droplet, micelle, reverse micelle, or lipid-polymer hybrid nanoparticle, or a mixture of two or more of the foregoing. The LV can be selected for a core that can carry a desired cargo. The LVs may be synthetic (artificially created or non-naturally occurring) or naturally occurring. Naturally occurring LVs may be in an isolated state (fully or partially isolated from their natural milieu) or in a non-isolated state. The LVs may be any shape but are typically spherical.

Although LVs have emerged as therapeutic carriers, the major limitation of using LVs has been the lack of loading of any cargos into preformed LVs and the lack of a well-developed methodology for increasing cellular uptake of their intended content(s). The present invention facilitates loading of LVs with cargo using CPPs and delivery of the cargo to recipient cells in vitro or in vivo.

LVs may be unilamellar in structure (having a single lipid layer) or multilamellar in structure (a concentric arrangement of two or more lipid layers). LVs may be spherical or have a non-spherical or irregular, heterogeneous shape. Examples of LVs include liposomes, lipid nanoparticles, lipid droplets, micelles, reverse micelles, and lipid-polymer hybrid nanoparticles. The surrounding lipid layer of LVs may be composed of synthetic lipids (e.g., a lipid manufactured by chemical synthesis from specified starting materials), semi-synthetic lipids (e.g., a lipid manufactured by modification of naturally occurring precursors such as dipalmitoylphosphatidylcholine (DPPC), distearoylphosphatidylcholine (DSPC), or dimyristoylphosphatidylcholine (DMPC)), naturally occurring lipids, or a combination of two or more of the foregoing, that are compatible with the lipid bilayer structure. In some embodiments, the lipid is a monoglyceride, diglyceride, or triglyceride, or a combination of two or more of the foregoing. Examples of lipids include phospholipids (such as phosphatidylcholine) and egg phosphatidylethanolamine.

Lipid nanoparticles or LNPs have a solid lipid core matrix surrounded by a lipid monolayer (Puri A et al., “Lipid-Based Nanoparticles as Pharmaceutical Drug Carriers: From Concepts to Clinic”, Crit Rev Ther Drug Carrier Syst, 2009; 26(6): 523-580; Saupe A and T Rades, “Solid Lipid Nanoparticles”, Nanocarrier Technologies, In: Mozafari M. R. (eds) Nanocarrier Technologies, 2006, p. 4; and Jenning, V et al., “Characterisation of a novel solid lipid nanoparticle carrier system based on binary mixtures of liquid and solid lipids”, International Journal of Pharmaceutics, 2000, 199(2):167-77). The LNP core is stabilized by surfactants and can solubilize lipophilic molecules. The core lipids can be fatty acids, acylglycerols, waxes, and mixtures of these surfactants. By “solid,” it is meant that at least a portion of the LNP are solid at room or body temperature and atmospheric pressure. However, an LNP can include portions of liquid lipid and/or entrapped solvent. Formulation methods for LNPs include high shear homogenization and ultrasound, solvent emulsification/evaporation, or microemulsion. Obtaining size distributions in the range of 30-180 nm is possible using ultrasonification at the cost of long sonication time. Solvent-emulsification is suitable in preparing small, homogeneously-sized lipid nanoparticles dispersions with the advantage of avoiding heat (Mehnert W, and K. Mader, “Solid lipid nanoparticles: Production, characterization and applications,” Advanced Drug Delivery Reviews, 2012, Volume 64, Pages 83-101).

A liposome is a vesicle having an interior aqueous core surrounded by, and enclosed by, at least one lipid bilayer (Akbarzadeh A et al., “Liposome: classification, preparation, and applications”, Nanoscale Res Lett. 2013; 8(1): 102; Wagner A and K Vorauer-Uhl, “Liposome Technology for Industrial Purposes”, Journal of Drug Delivery, 2011, Volume 2011, Article ID 591325, 9 pages).

Liposomes are typically spherical in shape but their shape and size may be controlled by their components, cargo, and preparation methods (Kawamura J et al., “Size-Controllable and Scalable Production of Liposomes Using a V-Shaped Mixer Micro-Flow Reactor”, Org. Process Res. Dev., 2020, 24, 10, 2122-2127; Miyata H and Hotani, “Morphological changes in liposomes caused by polymerization of encapsulated actin and spontaneous formation of actin bundles (cytoskeleton)”, Proc. Natl. Acad. Sci. USA, December 1992, Vol. 89, pp. 11547-11551; Yager P et al., “Changes in size and shape of liposomes undergoing chain melting transitions as studied by optical microscopy”, Biochimica et Biophysica Acta (BBA)—Biomembranes, 22 Dec. 1982, Volume 693, Issue 2, Pages 485-491).

In a liposome delivery product, the cargo (e.g., CD24, with or without additional cargo molecules) is generally “contained” in liposomes. The word “contained” in this context includes both encapsulated and intercalated cargo. The term “encapsulated” refers to cargo within an aqueous space and “intercalated” refers to incorporation of the cargo within a bilayer. Typically, water soluble cargos are contained in the aqueous compartment(s) and hydrophobic cargos are contained in the lipid bilayer(s) of the liposomes.

A liposome drug formulation is different from (1) an emulsion, which is a dispersed system of oil-in-water, or water-in-oil phases containing one or more surfactants, (2) a microemulsion, which is a thermodynamically stable two phase system containing oil or lipid, water, and surfactants, and (3) a drug-lipid complex.

Liposome structural components typically include phospholipids or synthetic amphiphiles incorporated with sterols, such as cholesterol, to influence membrane permeability. Thin-film hydration is a widely used preparation method for liposomes, in which lipid components with or without cargo are dissolved in an organic solvent. The solvent will be evaporated by rotary evaporation followed by rehydration of the film in an aqueous solvent. Other preparation methods include, for example, reverse-phase evaporation, freeze-drying and ethanol injection (Torchilin, V and V Weissig, “Liposomes: A Practical Approach”, Oxford University Press: Kettering, UK, 2003, pp. 77-101). Techniques such as membrane extrusion, sonication, homogenization and/or freeze-thawing are being employed to control the size and size distribution. Liposomes can be formulated and processed to differ in size, composition, charge, and lamellarity.

The major types of liposomes are the multilamellar vesicle (MLV, with multiple lamellar phase lipid bilayers), the small unilamellar liposome vesicle (SUV, with one lipid bilayer), the large unilamellar vesicle (LUV), and the cochleate vesicle. Some liposomes are multivesicular, in which one vesicle contains one or more smaller vesicles.

Liposome technology has been successfully translated into clinical applications. Delivery of therapeutics by liposomes alters their biodistribution profile, which can enhance the therapeutic index of drugs. Therapeutic areas in which lipid-based products have been used include, but are not limited to, cancer therapy (Doxil®, DaunoXome®, Depocyte®, Marqibo®, Myocet®, and Onivyde™), fungal diseases (Abelcet®, Ambisome®, and Amphotec®), analgesics (DepoDur™ and Exparel®), viral vaccines (Epaxal® and Inflexal® V), and photodynamic therapy (Visudyne®) (Bulbake U et al., “Liposomal Formulations in Clinical Use: An Updated Review”, Pharmaceutics, 2017, 9 (2):12; and Puri A et al. (2009). The invention may be used to load these agents into their respective liposomes, as well as a variety of other cargo-liposome combinations. Examples of lipid components used clinically in liposome-based products and in clinical trials can be found, for example, in Tables 1 and 2 of Bulbake U et al. (2017), which are incorporated herein by reference in their entirety.

The invention may be used with a variety of liposomal platforms, such as “stealth liposomes” (e.g., PEGylated liposomes), non-PEGylated liposomes, multivesicular liposomes (e.g., DepoFoam™ extended-release technology), and thermosensitive liposomes. In the case of DepoFoam™ extended-release technology, each particle contains numerous non-concentric aqueous chambers bounded by a single bilayer lipid membrane. Each chamber is partitioned from the adjacent chambers by bilayer lipid membranes composed of synthetic analogs of naturally existing lipids (DOPC, DPPG, cholesterol, triolein, etc.) (Murry D J and S M Blaney, “Clinical pharmacology of encapsulated sustained-release cytarabine”, Ann. Pharmacother., 2000, 34:1173-1178). Upon administration, DepoFoam™ particles release the drug over a period of time (hours to days) following erosion and/or reorganization of the lipid membranes.

Whereas liposomes are composed of a lipid bilayer separating an aqueous internal compartment from the bulk aqueous phase, micelles are closed lipid monolayers with a fatty acid core and polar surface, or polar core with fatty acids on the surface (reverse micelle).

The LV may be a lipid-polymer hybrid nanoparticle or “LPHNP”, which refers to a lipid vesicle having a polymer core that can contain cargo, with the polymer core encapsulated by a lipid monolayer (Mukherjee et al., “Lipid-polymer hybrid nanoparticles as a next-generation drug delivery platform: state of the art, emerging technologies, and perspectives”, Int J Nanomedicine, 2019 14:1937-1952).

The LV may be a lipid droplet, which is a cellular organelle containing a neutral-lipid core enclosed by a phospholipid monolayer (and associated proteins), and may be isolated from cells.

### Cell-Penetrating Polypeptides (CPPs)

In the past several decades, there have been many basic and preclinical research reports focused on the abilities of CPPs to carry and translocate various types of cargo molecules across the cellular plasma membrane. The inventors have determined that CPPs may be used to load vesicles such as EVs and LVs with a cargo molecule, and the loaded vesicles may then be used to deliver the cargo molecules comprising CD24 or a biologically active fragment or variant thereof to desired cells. The loaded cargo molecule may be carried by the EV or LV in or on the vesicle's outer layer or membrane (“membrane cargo”) or within the core of the vesicle (“luminal cargo”). CPPs disclosed herein may be coupled to cargo for loading EVs or LVs, and/or the CPPs may be coupled to the lipid surface of the EVs or LVs to target cells, cellular compartments, tissues, or organs.

Structurally, CPPs tend to be small natural or artificial peptides composed of about 5 to 30 amino acids; however, they may be longer. As used herein, the terms “cell penetrating polypeptide” and “CPP” refer to amino acid sequences of any length that have the membrane-traversing carrier function and are inclusive of short peptides and full-length proteins. CPPs may be any configuration, such as linear or cyclic (Park SE et al., “Cyclic Cell-Penetrating Peptides as Efficient Drug Delivery Tools”, Mol. Pharmaceutics, 2019, 16, 9, 3727-3743; Dougherty PG et al., “Understanding Cell Penetration of Cyclic Peptides”, Chem. Rev., 2019, 119 (17):10241-10287; Song J et al., “Cyclic Cell-Penetrating Peptides with Single Hydrophobic Groups”, Chembiochem. 2019 Aug. 16; 20 (16):2085-2088).

The CPP may be linear or cyclic. The CPP may be composed of L-amino acids, D-amino acids, or a mixture of both. The CPP may be protein derived, synthetic, or chimeric.

Cargo molecules comprising CD24 or a biologically active fragment or biologically active variant thereof may be associated with the CPPs through chemical linkage via covalent bonds or through non-covalent binding interactions, for example. CPPs typically have an amino acid composition that either contains a high relative abundance of positively charged amino acids such as lysine or arginine or have sequences that contain an alternating pattern of polar, charged amino acids and non-polar, hydrophobic amino acids. These two types of structures are referred to as polycationic or amphipathic, respectively. In some embodiments, the CPP is an arginine-rich peptide, lysine-rich peptide, or both. Another class of CPPs is the hydrophobic peptide, containing only apolar residues with low net charge or hydrophobic amino acid groups that are crucial for cellular uptake.

In one aspect, the CPP is a peptide having from 4 to 40 amino acids, where the peptide includes from 4 to 40 arginine residues. In one aspect, the CPP is a peptide having from 4 to 15 amino acids, where the peptide has 4, 5, 6, 7, 8, 9, 10, 11, 12, 13, 14, or 15 arginine residues, where any value can be a lower and upper endpoint of a range (e.g., 6 to 13 arginine residues). In one aspect, the CPP is a peptide comprising or consisting of 8 to 12 arginine residues. In another aspect, the CPP is a peptide comprising or consisting of 9 arginine residues.

In some embodiments, the CPP is cationic, amphipathic, both cationic and amphipathic, or anionic.

Transactivating transcriptional activator (TAT), GRKKRRQRRRPPQ, from human immunodeficiency virus 1 (HIV-1), and Antennapedia penetratin, RQIKIWFQNRRMKWKK, were among the first CPPs to be discovered. Since then, the number of known CPPs has expanded considerably, and small molecule synthetic analogues and cyclized peptides with more effective protein transduction properties have been generated (Habault J et al., “Recent Advances in Cell Penetrating Peptide-Based Anticancer Therapies”, Molecules, 2019 Mar; 24 (5):927; Derakhshankhah H et al., “Cell penetrating peptides: A concise review with emphasis on biomedical applications,” Biomedicine & Pharmacotherapy, 2018, 108:1090-1096; Borrelli A et al., “Cell Penetrating Peptides as Molecular Carriers for Anti-Cancer Agents”, Molecules, 2018, 23:295; and Okuyama M et al., “Small-molecule mimics of an alpha-helix for efficient transport of proteins into cells”, Nature Methods., 2007, 4 (2):153-9, which are each incorporated herein by reference in their entireties).

In some embodiments, the CPP is 3 to 5 amino acids in length. In some embodiments, the CPP is 6 to 10 amino acids in length. In some embodiments, the CPP is 11 to 15 amino acids in length. In some embodiments, the CPP is 16 to 20 amino acids in length. In some embodiments, the CPP is 21 to 30 amino acids in length. In some embodiments, the CPP is over 30 amino acids in length.

In some embodiments, the CPP is cationic. In some embodiments, the CPP is amphipathic. In some embodiments, the CPP is anionic.

The CPPs may have chemical modifications in-sequence (e.g., beta-alanine, linkers (e.g., Ahx), amino isobutyric acid (Aib), L-2-naphthyalalnine, or ornithine), N-terminal modifications (e.g., free, biotinylation, acetylation, or stearylation), internal modifications, and/or C-terminal modifications (e.g., free or amidated).

In some embodiments, two or more CPPs (which may be identical or different CPPs) are fused to the same cargo molecule in order to enhance their vesicle penetration power or capability.

The website CPPsite 2.0 is the updated version of the cell penetrating peptides database (CPPsite): webs.iiitd.edu.in/raghava/cppsite/information.php. It is a manually curated database holding many entries on CPPs that may be utilized in the invention. The website includes fields on (i) diverse chemical modifications, (ii) in vitro/in vivo model systems, and (iii) different cargoes delivered by CPPs. The CCPsite 2.0 covers different types of CPPs, including linear and cyclic CPPs, and CPPs with non-natural amino acid residues. The CPPsite 2.0 includes detailed structural information on CPPs, such as predicted secondary and tertiary structures of CPPs, including the structure of CPPs having D-amino acids and modified residues such as ornithine and beta-alanine. The CPPsite 2.0 includes information on diverse chemical modifications of CPPs that may be employed, including endo modifications (e.g., acylation, amidation, stearylation, biotinylation), non-natural residues (e.g., ornithine, beta-alanine), side chain modifications, peptide backbone modifications, and linkers (e.g., amino hexanoic acid). All CPPs on the CPPsite 2.0 database have been assigned a unique id number, which is constant throughout the database. CPPs are organized and can be browsed by length (up to 5 amino acids, 6-10 amino acids), 11-15 amino acids, 16-20 amino acids, 21-30 amino acids, and over 30 amino acids), and by category, including peptide type (linear or cyclic), peptide class (cationic or amphipathic), peptide nature (protein derived, synthetic, or chimeric), and peptide chirality (L, D, or mixed).

Examples of CPPs that may be used in the invention are provided in Behzadipour Y and S Hemmati “Considerations on the Rational Design of Covalently Conjugated Cell Penetrating Peptides (CPPs) for Intracellular Delivery of Proteins: A Guide to CPP Selection Using Glucarpidase as the Model Cargo Molecule”, Molecules, 2019, 24:4318, which is incorporated herein by reference in its entirety, including but not limited to the supplementary tables, and particularly the 1,155 peptides of Table 51.

Examples of CPPs that may be used in the invention are provided in Table 2 below. In some embodiments, the CPP is one listed in Table 2 or specifically identified elsewhere herein (e.g., by amino acid sequence).

TABLE 2
Examples of Natural and Artificial Cell-Penetrating Polypeptides

| | |
|---|---|
| Poly arginine: R(nR)R (n > 2) | LCLRPVG |
| Poly D-arginine: n(D-R) | RKKRRQRRR |
| (n > 5; D-R, D-arginine) | |
| KRRRGRKKRR | RRRKKRRRRR |
| RQIKIWFQNRRMKWKK | KETWWETWWTEWSQPKKKRKV |
| GWTLNSAGYLLGKINLKALAALAKKIL | VQRKRQKLMP |
| RRGRKKRRKR | RRKKRRRRRG |
| RGRKKRRKRR | RKKRRRRRGG |
| GRKKRRKRRR | YARAAARQARA (used here) |
| KRRRGRKKRR | YARAAARQARAC |
| YGRKKRRQRRR | YARAAARQARAGC (used here) |
| RKKRRKRRRR | KKIFKKILKFL |
| KKRRKRRRRK | KKLFKKIVKY |
| KRRKRRRRKK | KLFFKKILKYL |
| RRRGRKKRRK | CYARAAARQARAC |
| RRKRRRRKKR | KLIFKKILKYLKVFTISGKIILVGK |
| RKRRRRKKRR | KRKRKKLFKKILK |
| KRRRRKKRRR | SFATRFIPSP |
| RRRRKKRRRR | YRQERRARRRRRRERER |
| ALKFGLKLAL | ALKLALKLCL |
| ALKLCLKLGL | ASISQLKRSF |
| CLKLALKLAL | CLKLGLKLGL |
| GLKLALKFGL | KLALKFGLKL |
| KLALKLALKL | KLCLKLALKL |
| KLALKLGLKL | LALKLALKLA |
| LGLKLALKLC | LKLALKLALK |
| GQAGRARAAC | AGRARAACKL |
| KLALKLGLKLALKLCLKLGLKLGLKLALK | GRARAACKLA |
| FGLK | |
| RARAACKLAL | ARAACKLALR |
| RAACKLALRL | RLNPGALRPA |
| QGARLRSARK | GARLRSARKV |
| RLRSARKVLR | LRSARKVLRA |
| RKVLRATLKR | RKVLRAKLKR |
| GDIMGEWGNEIFGAIAGFLGYGRKKRRQR | GRKKRWFRRRRMKWKK |
| RR | |
| RKKRWFRRRRPKWKK | RIKRRFRRLRPKWKK |
| Ac-GLWRALWRLLRSLWRLLWRA- | RRKKIWFRRLRMK |
| cysteamide | |
| FₓrFₓKFₓrFₓK (Fₓ: cyclohexylalanine; | FrFKFrFK |
| r: D-Arginine) | |
| PLILLRLLRGQF | PLIYLRLLRGQF |
| RRILLQLLRGQF | pliylrllrgqf (all residues: D-form) |
| cyclo(FNₐRRRRQ) | cyclo(fNₐRrRrQ) (f: D-phenylalanine) |
| (Nₐ: L-2-naphthylalanine) | |
| cyclo(FfNₐRrRrQ) | cyclo(ZRRRRQ) (Z: L-Aspartic acid |
| | decylamine amide) |
| cyclo(CRRRRRRRRC) (Cyclization via a | cyclo(CYGRKKRRQRRRC) |
| disulfide bond) | (Cyclization via a disulfide bond) |
| cyclo(RRRRR) | cyclo(RRRRRR) |
| Dodecanoyl-cyclo(RRRRR) | Dodecanoyl-cyclo(RRRRRR) |
| LSTAADMQGVVTDGMASGLDKDYLKPDD | SPANLDQIVSAKKPKIVQERLEKV |
| | IASA |
| LSTAADMQGVVTDGMASG | SFEVHDKKNPTLEIPAGATVDVTF |
| | IN |
| VKKKKIKAEIKI | GLFDIIKKIAESF |
| KGEGAAVLLPVLLAAPG | GFWFG |
| ACTGSTQHQCG | gamma-AApeptides |

Examples of cell-penetrating proteins that have the membrane-traversing carrier function, and thus considered CPPs, are listed below:

Tat from human immunodeficiency virus type 1 (M. Green and P. M. Loewenstein, “Autonomous functional domains of chemically synthesized human immunodeficiency virus tat trans-activator protein”, Cell, 1988 Dec. 23, 55(6), 1179-1188. doi: 10.1016/0092-8674(88)90262-0) (A. D. Frankel and C. O. Pabo, “Cellular uptake of the tat protein from human immunodeficiency virus”, Cell, 1988 Dec. 23, 55(6), 1189-1193. doi: 10.1016/0092-8674(88)90263-2):

MEPVDPRLEPWKHPGSQPKTACTNCYCKKCCFHCQVCFITKALGISYGR
KKRRQRRRAHQNSQTHQASLSKQPTSQPRGDPTGPKE

Antennapedia from Drosophila melanogaster (A. Joliot, C. Pernelle, H. Deagostini-Bazin, and A. Prochiantz, “Antennapedia homeobox peptide regulates neural morphogenesis”, Proc. Natl. Acad. Sci. U.S.A. 1991, 88, 1864-1868) (P. E. G. Thorén, D. Persson, M. Karlsson, and B. Norden, “The Antennapedia peptide penetratin translocates across lipid bilayers—the first direct observation”, FEBS Lett. 2000, 482, 265-268):

MTMSTNNCESMTSYFTNSYMGADMHHGHYPGNGVTDLDAQQMHHYSQNA
NHQGNMPYPRFPPYDRMPYYNGQGMDQQQQHQVYSRPDSPSSQVGGVMP
QAQTNGQLGVPQQQQQQQQQPSQNQQQQQAQQAPQQLQQQLPQVTQQVT
HPQQQQQQPVVYASCKLQAAVGGLGMVPEGGSPPLVDQMSGHHMNAQMT
LPHHMGHPQAQLGYTDVGVPDVTEVHQNHHNMGMYQQQSGVPPVGAPPQ
GMMHQGQGPPQMHQGHPGQHTPPSQNPNSQSSGMPSPLYPWMRSQFGKC
QERKRGRQTYTRYQTLELEKEFHFNRYLTRRRRIEIAHALCLTERQIKI
WFQNRRMKWKKENKTKGEPGSGGEGDEITPPNSPQ

VP22 from herpes simplex virus type 1 (G. Elliott and P. O'Hare, “Intercellular Trafficking and Protein Delivery by a Herpesvirus Structural Protein”, Cell, 1997, 88, 223-233) (L. A. Kueltzo, N. Normand, P. O'Hare, and C. R. Middaugh, “Conformational lability of herpesvirus protein VP22”, J. Biol. Chem. 2000, 275, 33213-33221):

MTSRRSVKSGPREVPRDEYEDLYYTPSSGMASPDSPPDTSRRGALQTRS
RQRGEVRFVQYDESDYALYGGSSSEDDEHPEVPRTRRPVSGAVLSGPGP
ARAPPPPAGSGGAGRTPTTAPRAPRTQRVATKAPAAPAAETTRGRKSAQ
PESAALPDAPASTAPTRSKTPAQGLARKLHFSTAPPNPDAPWTPRVAGF
NKRVFCAAVGRLAAMHARMAAVQLWDMSRPRTDEDLNELLGITTIRVTV
CEGKNLLQRANELVNPDVVQDVDAATATRGRSAASRPTERPRAPARSAS
RPRRPVE

CaP from brome mosaic virus (X. Qi, T. Droste, and C. C. Kao, “Cell-penetrating peptides derived from viral capsid proteins”, Mol. Plant-Microbe Interact. 2010, 24, 25-36. doi: 10.1094/MPMI-07-10-0147):

MSTSGTGKMTRAQRRAAARRNRRTARVQPVIVEPLAAGQGKAIKAIAGY
SISKWEASSDAITAKATNAMSITLPHELSSEKNKELKVGRVLLWLGLLP
SVAGRIKACVAEKQAQAEAAFQVALAVADSSKEVVAAMYTDAFRGATLG
DLLNLQIYLYASEAVPAKAVVVHLEVEHVRPTFDDFFTPVYR

YopM from Yersinia enterocolitica (C. Ritter, C. Buss, J. Scharnert, G. Heusipp, and M. A. Schmidt, “A newly identified bacterial cell-penetrating peptide that reduces the transcription of pro-inflammatory cytokines”. J. Cell Sci., 2010 July; 123, 2190-2198. doi: 10.1242/jcs.063016):

MFINPRNVSNTFLQEPLRHSSDLTEMPVEAENVKSKAEYYNAWSEWERN
APPGNGEQRGMAVSRLRDCLDRQAHELELNNLGLSSLPELPPHLESLVA
SCNSLTELPELPQSLKSLQVDNNNLKALSDLPPLLEYLGAANNQLEELP
ELQNSSFLTSIDVDNNSLKTLPDLPPSLEFLAAGNNQLEELSELQNLPF
LTAIYADNNSLKTLPDLPPSLKTLNVRENYLTDLPELPQSLTFLDVSDN
IFSGLSELPPNLYNLNASSNEIRSLCDLPPSLVELDVRDNQLIELPALP
PRLERLIASFNHLAEVPELPQNLKLLHVEYNALREFPDIPESVEDLRMD
SERVIDPYEFAHETIDKLEDDVFE

Artificial protein B1 (R. L. Simeon, A. M. Chamoun, T. McMillin, and Z. Chen, “Discovery and Characterization of a New Cell-Penetrating Protein”, ACS. Chem. Biol., 2013; 8, 2678-2687. doi: 10.1021/cb4004089):

MWFKREQGRGAVHRGGAHPGRAGRRRKRPQVQRVRRGRGRCHLRQADPE
VHLHHRQAARALAHPRDHPDLRRAVLQPLPRPHEAARLLQVRHARRLRP
GAHHLLQGRRQLQDPRRGEVRGRHPGEPHRAEGHRLQGGRQHPGAQAGV
QLQQPQRLYHGRQAEERHQGELQDPPQHRGRQRAAHRPLPAEHPHRRRP
RAAARQPLPEHPVRPEQRPQREARSHGPAGVRDRRRDHSRHGRGLNLE

30Kc19 from silkworm Bombyx mori. (J. H. Park, J. H. Lee, H. H. Park, W. J. Rhee, S. S. Choi, and T. H. Park, “A protein delivery system using 30Kc19 cell-penetrating protein originating from silkworm”, Biomaterials, 2012, 33, 9127-9134. doi: 10.1016/j .biomaterials.2012.08.063):

MKPAIVILCLFVASLYAADSDVPNDILEEQLYNSVVVADYDSAVEKSKH
LYEEKKSEVITNVVNKLIRNNKMNCMEYAYQLWLQGSKDIVRDCFPVEF
RLIFAENAIKLMYKRDGLALTLSNDVQGDDGRPAYGKDKTSPRVSWKLI
ALWENNKVYFKILNTERNQYLVLGVGTNWNGDHMAFGVNSVDSFRAQWY
LQPAKYDNDVLFYIYNREYSKALTLSRTVEPSGHRMAWGYNGRVIGSPE
HYAWGIKAF

Engineered +36 GFP (Cronican J J et al., “Potent Delivery of Functional Proteins into Mammalian Cells in Vitro and in Vivo Using a Supercharged Protein”, ACS Chem. Biol. 2010, 5, 8, 747-752; doi: 10.1021/cb1001153):

MGHHHHHHGGASKGERLFRGKVPILVELKGDVNGHKFSVRGKGKGDATR
GKLTLKFICTTGKLPVPWPTLVTTLTYGVQCFSRYPKHMKRHDFFKSAM
PKGYVQERTISFKKDGKYKTRAEVKFEGRTLVNRIKLKGRDFKEKGNIL
GHKLRYNFNSHKVYITADKRKNGIKAKFKIRHNVKDGSVQLADHYQQNT
PIGRGPVLLPRNHYLSTRSKLSKDPKEKRDHMVLLEFVTAAGIKHGRDE
RYK

Naturally supercharged human proteins, e.g. N-DEK (primary sequence shown below) (Cronican J J et al., “A Class of Human Proteins That Deliver Functional Proteins Into Mammalian Cells In Vitro and In Vivo”, Chem. Biol., 2011, 18(7): 833-838; doi: 10.1016/j.chembio1.2011.07.003):

MFTIAQGKGQKLCEIERIHFFLSKKKTDELRNLHKLLYNRPGTVSSLKK
NVGQFSGFPFEKGSVQYKKKEEMLKKFRNAMLKSICEVLDLERSGVNSE
LVKRILNFLMHPKPSGKPLPKSKKTCSKGSKKER

Optionally, a CPP may be utilized that carries cargo molecules to a particular intracellular compartment, such as the cytosol or particular organelle. For example, an organelle-specific CPP may be used, capable of carrying cargo molecules to an organelle, such as the nucleus, mitochondria, Golgi apparatus, endoplasmic reticulum, lysosome/endosome, etc. (Cerrato C P et al., “Cell-penetrating peptides with intracellular organelle targeting”, Review Expert Opin Drug Deliv., 2017 February; 14(2):245-255; Sakhrani N M and H Padh, “Organelle targeting: third level of drug targeting,” Drug Des Devel Ther. 2013, 7: 585-599, which are each incorporated herein by reference in their entireties).

### Vesicle-Mediated CD24 Delivery to Cells

Vesicles loaded with cargo comprising CD24 or a biologically active fragment or biologically active variant thereof may be administered to cells in vitro by contacting the cells with the loaded vesicles, and vesicles loaded with cargo may be administered to cells in vivo by administering the loaded vesicles to organisms having the recipient cells, such as human or non-human animals. For delivery to cells in vivo, the loaded vesicles are administered by any route appropriate to reach the desired cells. Examples of routes include but are not limited to, oral, rectal, nasal, topical (including buccal and sublingual), vaginal and parenteral (including subcutaneous, intramuscular, intravenous, intradermal, intrathecal and epidural), and the like. In some embodiments, the vesicles are administered as an inhalant to cells of the upper and/or lower respiratory system.

For therapy or prophylaxis of a condition in a subject (e.g., human or animal diseases, disorders, or conditions, inflammation, hyper-inflammation, cytokine storm, cancer, infectious diseases, genetic diseases, central nervous system disorders, etc.), it will be appreciated that the preferred route may vary with, for example, the condition in question and the health of the subject. In some embodiments, the vesicles are administered locally at an anatomic site where the recipient cells are found, such as in the subject's airway (e.g., lungs) or at the site of a tumor or infection. In other embodiments, the vesicles are administered systemically for delivery to cells that may be anatomically remote from the site of administration. In some embodiments, vesicles are administered orally, sublingually, nasally, rectally, parenterally, subcutaneously, intramuscularly, or intravascularly (e.g., intravenously).

In addition to vesicle-mediated delivery of cargo to mature or specialized cells, vesicles may be used to deliver cargo to immature progenitor cells or stem cells. Recipient cells can range in plasticity from totipotent or pluripotent stem cells (e.g., adult or embryonic), precursor or progenitor cells, to highly specialized cells, such as those of the central nervous system (e.g., neurons and glia). Stem cells and progenitor cells can be found in a variety of tissues, including embryonic tissue, fetal tissue, adult tissue, adipose tissue, umbilical cord blood, peripheral blood, bone marrow, and brain, for example.

As will be understood by one of skill in the art, there are over 200 cell types in the human body. Vesicles can be delivered to any of these cell types. For example, any cell arising from the ectoderm, mesoderm, or endoderm germ cell layers can be a recipient of vesicles and their loaded cargo molecules. Recipient cells may be natural or wild-type cells, or cells of a cell line, for example.

Table 1 is a non-limiting list of examples of cells to which cargo molecules such as CD24 can be delivered using the invention.

Optionally, vesicles may include a targeting agent (also referred to as a targeting ligand) that targets the vesicles to a cellular compartment, cell type, organ, or tissue. A ligand such as an antibody, antibody fragment, and/or peptide may be bound to the surface of the vesicle (to the outer lipid layer). The ligand has a binding partner that is more abundant in or on the target cellular compartment, cell type, tissue, or organ, allowing the vesicle to target a cellular compartment or bind to and fuse with a specific cell type, tissue, or organ and deliver the cargo into the target cellular compartment, cells, tissue, or organ.

For example, if the targeting agent is an antibody or antibody fragment, the binding partner may be the antibody's/fragment's corresponding target antigen. If the targeting agent is a polypeptide that serves as a ligand for a receptor, the binding partner may be the ligand's corresponding target receptor. In some embodiments, the target for the targeting agent is a protein that is over-expressed on one or more cancer cell types (e.g., a tumor-associated antigen). Strategies for targeting vesicles using targeting ligands are described in Puri et al. (2009), which are incorporated herein by reference. For example, a galactosylated conjugated DOPE lipid carrying an anti-cancer agent as cargo may be used to specifically target the asialo-glycoprotein receptor on hepatocellular carcinoma. Folate-targeted vesicles carrying anti-cancer agent as cargo may be used to target cells with folate receptors, such as tumor cells. For liver targeting, an LV with galactosylated or mannosylated lipids may be used.

A CPP may be covalently or non-covalently coupled to the outer lipid layer of the vesicle to target a cell type, cellular compartment, tissue, or organ. The CPP selected as a targeting agent may be the same or different from the CPP selected for loading cargo into the vesicle. The BR2 and TAT peptides are examples of CPPs that may be used to target LVs in this way. For example, the CPP BR2 may be used to form cancer cell-targeting liposomes (BR2-liposomes) to deliver anti-cancer agents (Zhang X et al., “Liposomes equipped with cell penetrating peptide BR2 enhances chemotherapeutic effects of cantharadin against hepatocellular carcinoma”, Drug Delivery, 2017, 24(1):986-998). A CPP such as TAT may be conjugated to lipids to form TAT-liposomes which exhibit enhanced cellular internalization for delivery of therapeutic agents (Torchilin VP et al., “TAT peptide on the surface of liposomes affords their efficient intracellular delivery even at low temperature and in the presence of metabolic inhibitors”, PNAS, Jul. 17, 2001, 98(15):8786-8791). A different CPP may be used to load cargo into the TAT-liposome.

### Hyper-Inflammation and Cytokine Storm

When administered to a human or non-human animal subject (in vivo administration), the CD24 delivery method may be used to reduce an inflammatory response in the subject.

In some embodiments of the CD24 delivery method, the loaded vesicle is administered to the subject to treat, prevent, or delay the onset of hyper-inflammation in the subject. In some embodiments, the loaded vesicle is administered to the subject to treat, prevent, or delay the onset of cytokine storm in the subject. The cytokine storm may be caused by an infectious agent (e.g., SARS-CoV-2 or a variant thereof), or may be a monogenic disorder or autoimmune disorder (e.g., auto-inflammatory disorder, primary or secondary hemophagocytic lymphohistiocytosis (HLH)), or may have an iatrogenic cause (e.g., CAR-T cell therapy, blinatumomab, other T-cell engaging immunotherapy, or gene therapy). In some embodiments, the cytokine storm is caused by cancer, one or more cancer therapies (e.g., chemotherapy, immunotherapy, radiotherapy), or both.

In some embodiments, the loaded vesicle is administered to a human or non-human animal subject to treat, prevent, or delay onset of inflammation from danger-associated molecular patterns, or inflammation from septic injuries, as described in U.S. Pat. No. 8,637,013 “Treatment of drug-related side effect and tissue damage by targeting the CD24-HMGB1-Siglec10 axis” (Liu Y et al., issued Jan. 28, 2014, which is incorporated herein by reference in its entirety).

In some embodiments of the CD24 delivery method, the inflammation, hyper-inflammation, or cytokine storm is caused by a human corona virus. In some embodiments, the human coronavirus is a common human coronavirus, such as type 229E, NL63, 0C43, and HKU1. In some embodiments, the human coronavirus is selected from among SARS-CoV-2, SARS-CoV, and MERS-CoV. In some embodiments, the human coronavirus is a genetic variant of SARS-CoV-2 that is a variant of interest, variant of concern, or variant of high consequence as defined by the Centers for Disease Control and Prevention (CDC) and/or the World Health Organization (WHO). In some embodiments, the human coronavirus is the B.1.1.7 variant, B.1.351 variant, P.1 variant, B.1.427 variant, B.1.429 variant, or B.1.630 variant. In some embodiments, the human coronavirus is a variant such as the Iota variant (also known as the B.1.526 variant), B.1.526.1 variant, Eta variant (also known as the B.1.525 variant), P.2 variant, Kappa variant (also known as the B.1.617.1 variant), Lambda variant (also known as the C.37 variant), or the Mu variant (also known as the B.1.621 variant). In some embodiments, the human coronavirus is the Alpha variant (B.1.1.7), Beta variant (e.g., B.1.351, B.1.351.2, and B.1.351.3), Delta variant (e.g., B.1.617.2, AY.1, AY.2, AY.3, AY.4, and AY.4.2), or Gamma variant (e.g., P.1, P.1.1, and P.1.2).

The CD24 delivery method may be used in combination with other methods, including administration of additional agents. For example, if the loaded vesicle is administered to a subject to treat, prevent, or delay the onset of inflammation, hyper-inflammation, or cytokine storm, antibody therapies and blood-purification techniques (BPT) may be utilized. Examples of antibodies include tocilizumab, sarilumab, siltuximab, sotrovimab, casirivimab and imdevimab (administered together), and bamlanivimab and etesevimab (administered together). Examples of BPT include therapeutic plasma exchange (TPE), absorption, perfusion, and blood plasma filtration.

In some embodiments, in cases in which the loaded vesicle is administered to a subject to treat, prevent, or delay the onset of inflammation, hyper-inflammation, or cytokine storm, a treatment is administered to the subject that targets the causative agent of the inflammation, hyper-inflammation, or cytokine storm, as a two-pronged approach. For example, if the loaded vesicle is administered to a subject to treat, prevent, or delay the onset of inflammation, hyper-inflammation, or cytokine storm, and the causative agent is a microbial agent such as a virus or a bacteria, an agent such as an anti-viral agent or anti-bacterial agent that targets the causative microbial agent may be administered. In the case of SARS-CoV-2 or a variant thereof, for example, an anti-viral agent having a mechanism of action that inhibits one or more of viral cell entry, viral protease, host cell protease, virus membrane fusion, viral polyprotein cleavage, RNA-dependent RNA polymerase, or viral replication are options (Ghanbari R et al., “Existing antiviral options against SARS-CoV-2 replication in COVID-19 patients”, Future Microbiology, 2020, 15(18):1747-1758, which is incorporated herein by reference in its entirety).

In some embodiments, the loaded vesicle is administered to a subject to treat, prevent, or delay the onset of inflammation, hyper-inflammation, or cytokine storm, and the method further comprises administering a second agent, such as a drug or antibody, to the subject before, during, and/or after administration of the loaded vesicle. In particular embodiments, the second agent is an antiviral drug, a monoclonal antibody, a steroid, COVID-19 convalescent plasma, or a combination thereof. In further embodiments, the monoclonal antibody treatment is casirivimab, imdevimab, AZD7442 (a combination of long-acting antibodies (LAAB) tixagevimab (AZD8895) and cilgavimab (AZD1061)), and/or bamlanivimab. In yet another embodiment, the antiviral drug is remdesivir, chloroquine, hydroxychloroquine, and/or favipiravir. In particular embodiments, the second agent is at least one selected from the group consisting of amikacin, amphotericin formulations, atovaquone, any azole-containing anti-fungal drug, Bactrim, clindamycin, corticosteroids, echinocandin, fluconazole, flucytosine, itraconazole, posaconazole, quinine, sulfa drugs, trimethoprimsulfamethoxazole, voriconazole, baricitinib, interleukin-6 inhibitors, kinase inhibitors, tyrosine kinsase inhibitors, tocilizumab, ivermectin, or any FDA-approved drug for use in any coronavirus infection, and any combination thereof.

In some embodiments, the loaded vesicle is administered to a subject to treat, prevent, or delay the onset of inflammation, hyper-inflammation, or cytokine storm caused by a viral agent such as SARS-CoV-2 or a variant thereof, and the second agent administered before, during, and/or after administration of the loaded vesicle is one or more selected from among: griffisthin, nafamostat, lopinavir/ritonamir, PF-07321332 (a SARS-CoV-2-3CL protease inhibitor), nelfinavir, danoprevir, favipiravir, ribavirin, remdesivir, molnupiravir, AT-527 (a double prodrug of a guanosine nucleotide analog), and galidesivir.

The CD24 delivery method may include detecting the presence of a human coronavirus infection prior to administration of the loaded vesicle. Any suitable method for diagnosis or testing of coronavirus such as SARS-CoV-2 infection or a variant can be used, and such methods are well known in the art, including nucleic acid assays.

In some embodiments, the loaded vesicle is administered to a subject infected by a human coronavirus, such as SARS-CoV-2 or a variant thereof, as therapy. In some embodiments, the subject has the disease COVID-19.

In particular embodiments, the subject is symptomatic for a coronavirus infection (e.g., SARS-CoV-2 or variant infection) at the time of the administration. In other embodiments, the subject has tested positive for the coronavirus infection at the time of the administration but is asymptomatic. In further embodiments, the method further comprises administering a second agent, such as a drug and/or antibody, to the subject.

In other embodiments, the loaded vesicle is administered to a subject not infected by human coronavirus, such as SARS-CoV-2 or a variant thereof, as prophylaxis (to prevent or delay the onset of human coronavirus infection).

In particular embodiments, the subject is asymptomatic for the coronavirus infection and/or has been diagnosed as corona virus negative (e.g., SARS-CoV-2 negative) at the time of the administration. In other particular embodiments, the subject has been exposed to coronavirus (e.g., SARS-CoV-2 or variant) or has had close contact with someone infected with the coronavirus (e.g., SARS-CoV-2 or variant). In further embodiments, the method further comprises administering a second agent, such as a drug and/or antibody, to the subject.

In addition to treatment, prevention, or delay of onset of inflammation, hyper-inflammation, or cytokine storm, the invention may be used for delivery of CD24 or a biologically active fragment or variant thereof to cells in vitro or in vivo for any purpose that the delivered CD24 molecule may be useful for. For example, delivery of CD24 may be used to lower low density lipoprotein (LDL) cholesterol (LDL-C) in a subject, as described in U.S. Patent Publication 2018/0110828 (Liu Y and P Zheng; published Apr. 26, 2018; Oncolmmune, Inc.). Other uses include, for example, treatment of rheumatoid arthritis as described in U.S. Patent Application Publication US 2013/0231464 (Zheng et al., “Methods of Use of Soluble CD24 for Therapy of Rheumatoid Arthritis”, published Sept. 5, 2013), treatment of multiple sclerosis as described in U.S. Pat. No. 7,744,894 (Liu et al., “Method of Treating Multiple Sclerosis and Related T-Cell Initiated Tissue Destruction by Administering HSA/CD24”, issued Jun. 29, 2010). The treatment methods of U.S. Patent Publication 2018/0110828; US 2013/0231464; and U.S. Pat. No. 7,744,894 are incorporated herein by reference in its entirety.

### Further Definitions

As used herein, the terms “a,” “an,” “the” and similar terms used in the context of the present invention (especially in the context of the claims) are to be construed to cover both the singular and plural unless otherwise indicated herein or clearly contradicted by the context. Thus, for example, reference to “a cell” or “a cargo molecule” should be construed to cover both a singular cell or singular cargo molecule and a plurality of cells and a plurality of cargo molecules unless indicated otherwise or clearly contradicted by the context.

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “an excipient” include, but are not limited to, mixtures or combinations of two or more such excipients, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range. Thus, for example, if a component is in an amount of about 1%, 2%, 3%, 4%, or 5%, where any value can be a lower and upper endpoint of a range, then any range is contemplated between 1% and 5% (e.g., 1% to 3%, 2% to 4%, etc.).

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

As used herein, the terms “optional” or “optionally” means that the subsequently described event or circumstance can or cannot occur, and that the description includes instances where said event or circumstance occurs and instances where it does not.

Unless otherwise specified, temperatures referred to herein are based on atmospheric pressure (i.e. one atmosphere).

As used herein, the term “administration” is intended to include, but is not limited to, the following delivery methods: topical, oral, parenteral, subcutaneous, transdermal, transbuccal, intravascular (e.g., intravenous or intra-arterial), intramuscular, subcutaneous, intranasal, and intra-ocular administration. Administration can be local at a particular anatomical site, or systemic.

As used herein, the term “antibody” refers to whole antibodies and any fragment or portion of the whole antibody, such as an antigen binding fragment (i.e., “antigen-binding portion”) or single chains thereof. A whole antibody is a glycoprotein comprising at least two heavy (H) chains and two light (L) chains inter-connected by disulfide bonds. Each heavy chain comprises a heavy chain variable region (VH) and a heavy chain constant region comprising three domains, CH1, CH2 and CH3. Each light chain comprises a light chain variable region (VL or Vk) and a light chain constant region comprising one single domain, CL. The VH and VL regions can be further subdivided into regions of hyper-variability, termed complementarity determining regions (CDRs), interspersed with more conserved framework regions (FRs). Each VH or VL comprises three CDRs and four FRs, arranged from amino- to carboxy-terminus in the following order: FR1, CDR1, FR2, CDR2, FR3, CDR3, and FR4. The variable regions contain a binding domain that interacts with an antigen. The constant regions may mediate the binding of the antibody to host tissues or factors, including various cells of the immune system (e.g., effector cells) and the first component (Clq) of the classical complement system. An antibody is said to “specifically bind” to an antigen X if the antibody binds to antigen X with a K_{D} of 5×10⁻⁸ M or less, more preferably 1×10⁻⁸ M or less, more preferably 6×10⁻⁹ M or less, more preferably 3×10⁻⁹ M or less, even more preferably 2×10⁻⁹ M or less. The antibody can be chimeric, humanized, or, preferably, human. The heavy chain constant region can be engineered to affect glycosylation type or extent, to extend antibody half-life, to enhance or reduce interactions with effector cells or the complement system, or to modulate some other property. The engineering can be accomplished by replacement, addition, or deletion of one or more amino acids or by replacement of a domain with a domain from another immunoglobulin type, or a combination of the foregoing. The antibody may be any isotype, such as IgM or IgG. An antibody fragment or antibody portion may also refer to the relatively constant or Fc portion of the antibody.

As used herein, the terms “antigen-binding fragment” and “antigen-binding portion” of an antibody refer to one or more fragments of an antibody that retain the ability to specifically bind to an antigen. It has been shown that the antigen-binding function of an antibody can be performed by fragments of a full-length antibody, such as (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fab′ fragment, which is essentially an Fab with part of the hinge region (see, for example, Abbas et al., Cellular and Molecular Immunology, 6th Ed., Saunders Elsevier 2007); (iv) an Fd fragment consisting of the VH and CH1 domains; (v) an Fv fragment consisting of the VL and VH domains of a single arm of an antibody, (vi) a dAb fragment (Ward et al., Nature, 1989, 341:544-546), which consists of a VH domain; (vii) an isolated complementarity determining region (CDR); and (viii) a nanobody, a heavy chain variable region containing a single variable domain and two constant domains. Furthermore, although the two domains of the Fv fragment, VL and VH, are encoded by separate genes, they can be joined, using recombinant methods, by a synthetic linker that enables them to be made as a single protein chain in which the VL and VH regions pair to form monovalent molecules (known as single chain Fv, or scFv); see, e.g., Bird et al. (1988) Science 242:423-426; and Huston et al. (1988) Proc. Natl. Acad. Sci. USA 85:5879-5883). Such single chain antibodies are also encompassed within the term “antigen-binding portion” or “antigen-binding fragment” of an antibody.

As used herein, the term “CD24 molecule” refers to a CD24 polypeptide of a human or non-human animal, or a biologically active fragment or biologically active variant thereof, or a nucleic acid sequence encoding such polypeptide. The CD24 polypeptide may comprise a canonical sequence or an isoform. The CD24 polypeptide may be synthetic (synthesized) or recombinantly produced. A CD24 polypeptide may be a full-length protein or a mature polypeptide or sub-sequence that retains CD24 biological activity. Representative examples of “biological activity” in the context of CD24 molecules include the ability to do one or more of the following: binding molecules that exhibit DAMPs; participating in the formation of a trimolecular complex composed of the CD24 molecule; DAMPs and Siglec (such as and Siglec G (mouse) or Signlec 10 (human) form); recruitment of Src family protein tyrosine kinases (PTKs) such as c-fgr, lyn and lck via membrane rafts; activating the mitogen-activated protein kinase pathway; and binding antibodies that specifically bind other CD24 molecules (Fang X et al., “CD24: from A to Z”, Cell Mol Immunol, 2010, 7:100-103).

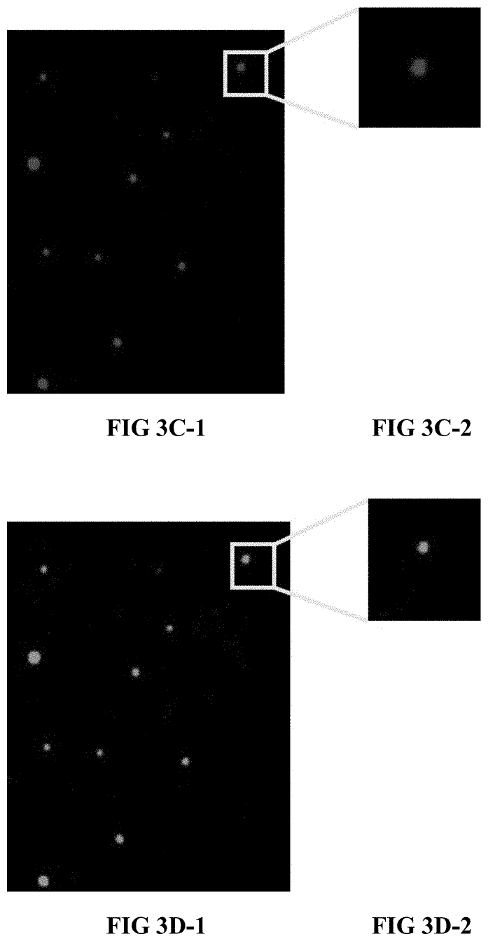

In some embodiments, the CD24 molecule may have the amino sequence of mature human CD24, which may be SETTTGTSSNSSQSTSNSGLAPNPTNATTK or SETTTGTSSNSSQSTSNSGLAPNPTNATTK(V/A), or mouse CD24, which may be NQTSVAPFPGNQNISASPNPTNATTRG, or a biologically active fragment or biologically active variant thereof. The CD24 may be soluble. The CD24 may further comprise a N-terminal signal peptide, which may have the amino acid sequence MGRAMVARLGLGLLLLALLLPTQIYS. The CD24 may also have an amino acid sequence described in FIG. 1 or FIG. 3 of U.S. Patent Application Publication US 2013/0231464 (Zheng et al., “Methods of Use of Soluble CD24 for Therapy of Rheumatoid Arthritis”, published Sep. 5, 2013, which is incorporated herein by reference in its entirety). The CD24 may exist in one of two allelic forms, such that the C-terminal amino acid of the mature human CD24 may be a valine or an alanine. The C-terminal valine or alanine may be immunogenic and may be omitted from the CD24 to reduce its immunogenicity.

The term “CD24 molecule” is inclusive of fusion proteins that include a CD24 polypeptide of a human or non-human animal, or a biologically active fragment or variant such polypeptides. The CD24 polypeptide, fragment, or variant may be fused at its N- or C-terminal end to a portion of a mammalian Ig protein, which may be human or mouse, for example. The portion may be a Fc region of the Ig protein. The Fc region may comprise the hinge region and CH2 and CH3 domains of the Ig protein. The Ig protein may be human IgG1, IgG2, IgG3, IgG4, IgM, or IgA. The Ig protein may also be IgM, and the Fc portion may comprise the hinge region and CH3 and CH4 domains of IgM. The CD24 may also be fused at its N- or C-terminus to a protein tag, which may be GST, His, or FLAG. Methods for making fusion proteins and purifying fusion proteins are well known in the art.

As used herein, the term “cell penetrating polypeptide” or “CPP” refers to a polypeptide of any length having the ability to cross cellular membranes with a cargo molecule. These polypeptides are sometimes referred to as cell penetrating peptides, cell penetrating proteins, transport peptides, carrier peptides, and peptide transduction domains. The CPPs used in the invention have the capability, when coupled to a cargo molecule, of facilitating entrapment of a cargo molecule by a vesicle (e.g., EV or LV). The loaded cargo molecule may be carried by the vesicle in or on the vesicle's outer lipid layer or membrane (“membrane cargo”) or within the core of the vesicle (“luminal cargo”). Structurally, CPPs tend to be small peptides, typically about 5 to 30 amino acids in length, though they may be longer. As used herein, the terms “cell penetrating polypeptide” and “CPP” are inclusive of short peptides and full-length proteins having the membrane-traversing carrier function. CPPs may be any configuration, such as linear or cyclic, may be artificial or naturally occurring, may be synthesized or recombinantly produced, and may be composed of traditional amino acids or may include one or more non-traditional amino acids. A non-exhaustive list of examples of CPPs is provided in Table 2.

As used herein, the term “contacting” in the context of contacting a cell with a loaded vesicle of the invention (e.g., EV or LV) in vitro or in vivo means bringing at least one loaded vesicle into contact with the cell, or vice-versa, or any other manner of causing the loaded vesicle and the cell to come into contact.

As used herein, the term “cytokine storm” (also called hypercytokinemia, cytokine release syndrome, or cytokine cascade) is an umbrella term encompassing several disorders of immune dysregulation characterized by constitutional symptoms, systemic inflammation, and multi-organ dysfunction that can lead to multi-organ failure and even death if inadequately treated. On a basic level, cytokine storm involves an immune response that causes collateral damage which may be greater than the immediate benefit of the immune response, and can be defined as a physiological reaction in humans and non-human animals in which the innate immune system causes an uncontrolled and excessive release of pro-inflammatory signaling molecules called cytokines. Patients with cytokine storm typically have the following three conditions: (1) elevated circulating cytokine levels, such as interleukin-1 (IL-1) and IL-6, (2) acute systemic inflammatory symptoms, and (3) secondary organ dysfunction (e.g., renal, hepatic, or pulmonary) (Fajgenbaum D C et al., 2020).

A wide range of clinical and laboratory abnormalities can be observed in cytokine storm. A large number of cytokines, chemokines, and other mediators involved in cytokine storm have been identified, such as IL-1, IL-2, IL-6, IL-9, IL-10, IL-12, IL-17, IL-18, IL-33, interferon-gamma, tumor necrosis factor, GM-CSF, VEGF, IL-8 (CXCL-8), MIG (CCL9), MIP-1β (CCL4), complement, and ferritin (Fajgenbaum D C etal., 2020, particularly Table 1).

The onset and duration of cytokine storm vary, depending on the cause and treatments administered. Although the initial causes may differ, late-stage clinical manifestations of cytokine storm converge and often overlap. Most patients with cytokine storm are febrile, with high grade fever in severe cases (Fajgenbaum D C et al., 2020). In addition, patients may have fatigue, anorexia, headache, rash, diarrhea, arthralgia, myalgia, and neuropsychiatric findings (Ye Q et al., “The pathogenesis and treatment of the ‘Cytokine Storm’ in COVID-19, Journal of Infection, 2020, 80:6-7-613). These symptoms may be due directly to cytokine-induced tissue damage or acute-phase physiological changes or may result from immune-cell—mediated responses. Cases can progress rapidly to disseminated intravascular coagulation with either vascular occlusion or catastrophic hemorrhages, dyspnea, hypoxemia, hypotension, hemostatic imbalance, vasodilatory shock, and death. Many patients have respiratory symptoms, including cough and tachypnea, that can progress to acute respiratory distress syndrome (ARDS), with hypoxemia that may require mechanical ventilation. The combination of hyperinflammation, coagulopathy, and low platelet counts places patients with cytokine storm at high risk for spontaneous hemorrhage (Fajgenbaum D C et al., 2020).

Cytokine storms can be caused by a number of infectious and non-infectious etiologies (Fajgenbaum D C et al., 2020, particularly Table 2), especially viral respiratory infections such as H5N1 influenza, H7N9 influenza, influenza B, parainfluenza virus, Ebola, Middle Eastern respiratory syndrome (MERS), and severe acute respiratory syndrome (SARS) such as SARS-CoV-1 and SARS-CoV-2 (causative agent for COVID-19 pandemic) (Tisoncik J R et al., “Into the Eye of the Cytokine Storm”, Microbiology and Molecular Biology Reviews, 2012, 76 (1): 16-32). Viral causative agents can invade lung epithelial cells and alveolar macrophages to produce viral nucleic acid, which stimulates the infected cells to release cytokines and chemokines, activating macrophages, dendritic cells, and immune cells (Song, P et al., “Cytokine storm induced by SARS-CoV-2”, Clinica Chimica Acta; International Journal of Clinical Chemistry, October 2020, 509: 280-287). Other causative agents include but are not limited to, the Epstein-Barr virus, cytomegalovirus, and group A streptococcus, and non-infectious conditions such as graft-versus-host disease, and some therapies such as chimeric antigen receptor (CAR)-expressing T cell (CAR-T cell) therapy CAR T-cell therapy, which is an autologous T cell therapy designed such that T cells taken from a patient are genetically engineered to express a targetable chimeric antigen receptor (CAR) and then returned into the patient's body (Chen H et al., “Management of cytokine release syndrome related to CAR-T cell therapy, Front Med, 2019, 13(5):610-617; and Zhang C et al., “Radiotherapy and Cytokine Storm: Risk and Mechanism”, Front. Oncol., May 2021, 11:670464).

Cytokine storm syndrome refers to a number of overlapping hyper-inflammatory clinical syndromes that can result in a cytokine storm. Cytokine storm syndromes include familial hemophagocytic lymphohistiocytosis (HLH), Epstein-Barr virus-associated hemophagocytic lymphohistiocytosis, macrophage activation syndrome such as systemic or non-systemic juvenile idiopathic arthritis-associated macrophage activation syndrome and NLR family CARD domain-containing protein 4 (NLRC4) macrophage activation syndrome, cytokine release syndrome (CRS), and sepsis (Manson J J et al., “COVID-19-associated hyperinflammation and escalation of patient care: a retrospective longitudinal cohort study,” Lancet Rheumatol, 2020 October; 2(10):e594-e602). In severe cases of CRS, patients can experience a cytokine storm, in which there is a positive feedback loop between cytokines and white blood cells with highly elevated levels of cytokines. This can lead to potentially life-threatening complications including cardiac dysfunction, adult respiratory distress syndrome, neurologic toxicity, renal and/or hepatic failure, pulmonary edema and disseminated intravascular coagulation.

Multisystem inflammatory syndrome (MIS-C) is a pediatric hyper-inflammation disorder caused by SARS-CoV-2. Some of the clinical manifestations of MIS-C mimic Kawasaki disease (KD) shock syndrome (Kabeerdoss J et al., “Severe COVID-19, multisystem inflammatory syndrome in children, and Kawasaki disease: immunological mechanisms, clinical manifestations and management”, Rheumatol Int, 2021, 41(1):19-32).

As used herein, the term “gene editing enzyme” refers to an enzyme having gene editing function, such as nuclease function. The gene editing enzyme may be, for example, a Zinc finger nuclease (ZFN), transcription-activator like effector nuclease (TALEN), meganuclease, or component of the Clustered Regularly Interspaced Short Palindromic Repeats (CRISPR) system. CRISPRs are genetic elements that bacteria and archaea use as an acquired immunity to protect against bacteriophages. They consist of short sequences that originate from bacteriophage genomes and have been incorporated into the bacterial genome. Cas (CRISPR associated proteins) process these sequences and cut matching viral DNA sequences. By introducing plasmids containing Cas genes and specifically constructed CRISPRs into eukaryotic cells, the eukaryotic genome can be cut at any desired position. CRISPR associated protein 9 (Cas9) is one example of a CRISPR gene editing enzyme that may be used with the invention. A small piece of RNA is created with a short guide sequence that binds to a specific target sequence of DNA in a genome. The RNA also binds to the Cas9 enzyme. As in bacteria, the modified RNA is used to recognize the DNA sequence, and the Cas9 enzyme cuts the DNA at the targeted location. As described below, although Cas9 is the enzyme that is used most often, other enzymes (for example, Cas12a (also known as Cpf1)) can also be used. Once the DNA is cut, the cell's own DNA repair machinery is used to add or delete pieces of genetic material, or to make changes to the DNA by replacing an existing segment with a customized DNA sequence.

Cas9 is the most well characterized Cas endonuclease and most often used in CRISPR laboratories; however, its use is often limited by its large size, its protospacer adjacent motif (PAM) sequence stringency, and its propensity to cut off-target DNA sequences. Many have addressed these limitations of Cas9 by engineering derivatives with more desirable properties, in particular increased specificity and reduced PAM stringency. Alternative Cas endonucleases with overlapping as well as unique properties may be used, such as Cas3, Cas12 (e.g., Cas12a, Cas12d, Cas12e), Cas13 (Cas13a, Cas13b), and Cas14. Depending upon the particular intended application, potentially any class, type, or subtype of CRISPR-Cas system may be used in the invention (Meaker GA and EV Koonen, “Advances in engineering CRISPR-Cas9 as a molecular Swiss Army knife”, Synth Biol (Oxf)., 2020; 5(1): ysaa021; Jamehdor S et al., “An overview of applications of CRISPR-Cas technologies in biomedical engineering”, Folia Histochemica et Cytobiologica, 2020, 58(3): 163-173; Zhu Y. and Zhiwei Huang, “Recent advances in structural studies of the CRISPR-Cas-mediated genome editing tools”, National Science Review, 2019, 6: 438-451; Murugan K et al., “The revolution continues: Newly discovered systems expand the CRISPR-Cas toolkit”, Mol Cell. 2017 Oct 5; 68(1): 15-25; and Makarova KS et al., “Annotation and Classification of CRISPR-Cas Systems”, Methods Mol Biol, 2015; 1311: 47-75, which are each incorporated herein by reference in their entireties).

As used herein, the term “human antibody” means an antibody having variable regions in which both the framework and CDR regions (and the constant region, if present) are derived from human germline immunoglobulin sequences. Human antibodies may include later modifications, including natural or synthetic modifications. Human antibodies may include amino acid residues not encoded by human germline immunoglobulin sequences (e.g., mutations introduced by random or site-specific mutagenesis in vitro or by somatic mutation in vivo). However, “human antibody” does not include antibodies in which CDR sequences derived from the germline of another mammalian species, such as a mouse, have been grafted onto human framework sequences.

As used herein, the term “humanized immunoglobulin” or “humanized antibody” refers to an immunoglobulin or antibody that includes at least one humanized immunoglobulin or antibody chain (i.e., at least one humanized light or heavy chain). The term “humanized immunoglobulin chain” or “humanized antibody chain” (i.e., a “humanized immunoglobulin light chain” or “humanized immunoglobulin heavy chain”) refers to an immunoglobulin or antibody chain (i.e., a light or heavy chain, respectively) having a variable region that includes a variable framework region substantially from a human immunoglobulin or antibody and complementarity determining regions (CDRs) (e.g., at least one CDR, preferably two CDRs, more preferably three CDRs) substantially from a non-human immunoglobulin or antibody, and further includes constant regions (e.g., at least one constant region or portion thereof, in the case of a light chain, and preferably three constant regions in the case of a heavy chain). The term “humanized variable region” (e.g., “humanized light chain variable region” or “humanized heavy chain variable region”) refers to a variable region that includes a variable framework region substantially from a human immunoglobulin or antibody and complementarity determining regions (CDRs) substantially from a non-human immunoglobulin or antibody.

As used herein, the term “human monoclonal antibody” refers to an antibody displaying a single binding specificity, which has variable regions in which both the framework and CDR regions are derived from human germline immunoglobulin sequences. In one embodiment, human monoclonal antibodies are produced by a hybridoma that includes a B cell obtained from a transgenic nonhuman animal, e.g., a transgenic mouse, having a genome comprising a human heavy chain transgene and a light chain transgene fused to an immortalized cell.

As used herein, the term “hyper-inflammation” refers to an uncontrolled, self-perpetuating, and tissue-damaging inflammatory activity (Manson J J et al.).

As used herein, the term “isolated antibody” means an antibody or antibody fragment that is substantially free of other antibodies having different antigenic specificities (e.g., an isolated antibody that specifically binds antigen X is substantially free of antibodies that specifically bind antigens other than antigen X). An isolated antibody that specifically binds antigen X may, however, have cross-reactivity to other antigens, such as antigen X molecules from other species. In certain embodiments, an isolated antibody specifically binds to human antigen X and does not cross-react with other (non-human) antigen X antigens. Moreover, an isolated antibody may be substantially free of other cellular material and/or chemicals.

As used herein, the terms “lipid vesicle” or “LV” refer to a naturally occurring or an artificially created (non-naturally occurring) particle having an interior compartment or cavity (core) surrounded and enclosed by at least one lipid layer (e.g., a lipid monolayer or a lipid bilayer). LVs may be unilamellar in structure (having a single lipid layer) or multilamellar in structure (a concentric arrangement of two or more lipid layers). LVs may be spherical or have a non-spherical or irregular, heterogeneous shape. Examples of LVs include liposomes, lipid nanoparticles, lipid droplets, micelles, reverse micelles, and lipid-polymer hybrid nanoparticles; thus, the term LV is inclusive of liposomes, lipid nanoparticles, lipid droplets, micelles, reverse micelles, and lipid-polymer hybrid nanoparticles. The surrounding lipid layer may be composed of synthetic lipids, semi-synthetic lipids, naturally occurring lipids, or a combination of two or more of the foregoing, which are compatible with the lipid layer structure. The term lipid is used in a broader sense and includes, for example, triglycerides (e.g., tristearin), diglycerides (e.g., glycerol bahenate), monoglycerides (e.g., glycerol monostearate), fatty acids (e.g., stearic acid), steroids (e.g., cholesterol), and waxes (e.g., cetyl palmitate).

As used herein, the term “liposome” refers to a vesicle having an interior aqueous core surrounded by, and enclosed by, at least one lipid bilayer. Liposomes are typically spherical in shape but their shape and size may be controlled by their components, cargo, and preparation methods. In a liposome delivery product, the cargo (e.g., CD24 or a drug substance) is generally “contained” in liposomes. The word “contained” in this context includes both encapsulated and intercalated cargo. The term “encapsulated” refers to cargo within an aqueous space and “intercalated” refers to incorporation of the cargo within a bilayer. Typically, water soluble cargos are contained in the aqueous compartment(s) and hydrophobic cargos are contained in the lipid bilayer(s) of the liposomes.

The major types of liposomes are the multilamellar vesicle (MLV, with multiple lamellar phase lipid bilayers), the small unilamellar liposome vesicle (SUV, with one lipid bilayer), the large unilamellar vesicle (LUV), and the cochleate vesicle. Some liposomes are multivesicular, in which one vesicle contains one or more smaller vesicles.

As used herein, the terms “lipid nanoparticle” or “LNP” and “solid lipid nanoparticle” or “SLNP” are interchangeable and refer to nanoparticles composed of lipids. LNPs have a solid lipid core matrix surrounded by a lipid monolayer. The LNP core is stabilized by surfactants and can solubilize lipophilic molecules. The core lipids can be fatty acids, acylglycerols, waxes, and mixtures of these surfactants. By “solid,” it is meant that at least a portion of the LNP is solid at room temperature or body temperature and atmospheric pressure. However, the LNP can include portions of liquid lipid and/or entrapped solvent.

As used herein, a “lipid droplet” refers to a cellular organelle containing a neutral-lipid core enclosed by a phospholipid monolayer (and associated proteins). Lipid droplets may be isolated from cells.

As used herein, the term “micelle” refers to an LV with a closed lipid monolayer and a fatty acid core and polar surface, whereas a “reverse micelle” or “inverted micelle” has a polar core with fatty acids on its surface.

Liposomes are composed of a lipid bilayer separating an aqueous internal compartment from the bulk aqueous phase. Micelles are closed lipid monolayers with a fatty acid core and polar surface, or polar core with fatty acids on the surface (inverted micelle).

As used herein, the term “lipid-polymer hybrid nanoparticles” or “LPHNP” refers to a lipid vesicle having a polymer core that can contain cargo, with the polymer core encapsulated by a lipid monolayer.

As used herein, a “lipid bilayer” refers to a structure composed of two layers of lipid molecules organized in two sheets, functioning as a barrier. A lipid bilayer surrounds cells as a biological membrane, providing the cell membrane structure. Liposomes have a lipid bilayer that creates an inner aqueous compartment due to the hydrophilic heads and the hydrophobic tails of the lipids.

As used herein, the term “monoclonal antibody” or “monoclonal antibody composition” means a preparation of antibody molecules of single molecular composition, which displays a single binding specificity and affinity for a particular epitope.

As used herein, the term “nucleic acid” means any DNA-based or RNA-based molecule, and may be a cargo molecule of the invention. The term is inclusive of polynucleotides and oligonucleotides. The term is inclusive of synthetic or semi-synthetic, recombinant molecules which are optionally amplified or cloned in vectors, and chemically modified, comprising unnatural bases or modified nucleotides comprising, for example, a modified bond, a modified purine or pyrimidine base, or a modified sugar. The nucleic acid may be in the form of single-stranded or double-stranded DNA and/or RNA. The nucleic acid may be a synthesized molecule, or isolated using recombinant techniques well-known to those skilled in the art. The nucleic acid may encode a polypeptide of any length, such as a CD24 polypeptide, or the nucleic acid may be a non-coding nucleic acid. The nucleic acid may be a messenger RNA (mRNA). The nucleic acid may be a morpholino oligomer. For nucleic acids encoding polypeptides, the nucleic acid sequence may be deduced from the sequence of the polypeptide and the codon usage may be adjusted according to the host cell in which the nucleic acid is to be transcribed. DNA encoding a polypeptide, such as a CD24 polypeptide, optionally includes a promoter operably linked to the encoding DNA for expression in cells in vitro or in vivo.

In some embodiments, the nucleic acid is a DNA or RNA having an enzymatic activity (e.g., a DNAzyme or RNAzyme). In some embodiments, the nucleic acid is a ribonucleic acid (RNA) enzyme that catalyzes chemical reactions. RNAzyme is usually an artificial enzyme derived from in vitro RNA evolution method such as SELEX. A ribozyme, also called catalytic RNA, is usually an RNA enzyme which forms a complex with protein(s) or exists in the RNA/protein complex, e.g. ribosome. In some embodiments, the nucleic acid is a catalytic RNA, RNAzyme, or ribozyme.

In some embodiments, the nucleic acid is an antisense oligonucleotide, DNA, interfering RNA molecule (e.g., shRNA), microRNA, tRNA, mRNA, guide RNA (e.g., sgRNA) for gene editing by a gene editing enzyme such as CRISPR Cas9, catalytic RNA, RNAzyme, or ribozyme.

In some embodiments, the nucleic acid is inhibitory, such as an antisense oligonucleotide. In some embodiments, the nucleic acid is an RNA molecule such as snRNA, ncRNA (e.g. miRNA), mRNA, tRNA, catalytic RNA, RNAzyme, ribozyme, interfering RNA (e.g., shRNA, siRNA), or guide RNA (e.g., sgRNA) for a gene editing enzyme such as CRISPR Cas9. In some embodiments, the nucleic acid is a peptide nucleic acid (PNA).

As used herein, the terms “patient”, “subject”, and “individual” are used interchangeably and are intended to include human and non-human animal species. For example, the subject may be a human or non-human mammal. In some embodiments, the subject is a non-human animal model or veterinary patient. For example, the non-human animal patient may be a mammal, reptile, fish, or amphibian. In some embodiments, the non-human animal is a dog, cat, mouse, rat, guinea pig. In some embodiments, the non-human animal is a primate. The subject may be any age or gender.

In the various embodiments, the human subject can be an adult or child. In some cases, the subject may be an infant or older adult. In some embodiments, the subject is 40 years of age or older. In some embodiments, the subject is 55 years of age or older. In some embodiments, the subject is 60 years of age or older. In some embodiments, the subject is an infant. As used herein, a “child” refers to a human subject who is between the ages of 1 day to <18 years of age. The term “adult” refers to a human subject who is 18 years of age or older. In particular embodiments, the human subject is an adult of advanced age, such as an adult of 55 years of age or greater, 56 years of age or greater, 57 years of age or greater, 58 years of age or greater, 59 years of age or greater, 60 years of age or great, 61 years of age or greater, 62 years of age or greater, 63 years of age or greater, 64 years of age or greater, 65 years of age or greater, 66 years of age or greater, 67 years of age or greater, 68 years of age or greater, 69 years of age or greater, 70 years of age or greater, 75 years of age or greater, or 80 years of age or greater.

As used herein, the terms “protein”, “polypeptide”, and “peptide” are used interchangeably to refer to a polymeric form of amino acids of any length, which can include coded and non-coded amino acids, natural amino acids, chemically or biochemically modified or derivatized amino acids, and polypeptides having modified peptide backbones. The term “polypeptide” includes full-length proteins and fragments or subunits of proteins. For example, in the case of enzymes, the polypeptide may be the full-length enzyme or an enzymatically active subunit or portion of the enzyme. The term “polypeptide” includes fusion proteins, including, but not limited to, fusion proteins with a heterologous amino acid sequence, fusions with one or more repeats of sequences (multimers), fusions with heterologous and homologous leader sequences, with or without N-terminal methionine residues; immunologically tagged proteins; and the like. The term “polypeptide” includes polypeptides comprising one or more of a fatty acid moiety, a lipid moiety, a sugar moiety, and a carbohydrate moiety. The polypeptide may be synthesized or recombinantly produced. The term “polypeptides” includes post-translationally modified polypeptides. The polypeptide may be a cargo molecule of the invention. The polypeptide may be a cell penetrating polypeptide (CPP) of the invention. The polypeptide may be CD24 of a human or non-human animal, or a biologically active fragment or biologically active variant of CD24.

The term “substantially identical” in the context of comparative amino acid sequences such as CD24 amino acid sequences or CPP amino acid sequences means that a first and second amino acid sequence are at least 60%, 65%, 70%, 75%, 80%, 85%, 90%, 95%, 96%, 97%, 98%,or 99% over a region of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, or 300 amino acids. One can use a program such as the CLUSTAL program to compare amino acid sequences. This program compares amino acid sequences and finds the optimal alignment by inserting spaces in either sequence as appropriate. It is possible to calculate amino acid identity or similarity (identity plus conservation of amino acid type) for an optimal alignment. A program like BLASTx will align the longest stretch of similar sequences and assign a value to the fit. It is thus possible to obtain a comparison where several regions of similarity are found, each having a different score. The above applied mutatis mutandis to all amino acid sequences disclosed in the present application.

As used herein, the phrase “therapeutically effective amount” or “efficacious amount” means the amount of an agent, such as a cargo molecule, that, when administered to a human or non-human animal subject for treating a disease, disorder, or condition, is sufficient, in combination with another agent, or alone in one or more doses, to effect such treatment for the disease, disorder, or condition. The “therapeutically effective amount” will vary depending on the agent, the disease, disorder, or condition, and its severity and the age, weight, etc., of the subject to be treated.

As used herein, the term “treat”, “treating” or “treatment” of any disease, disorder, or condition, such as hypercytokinemia, refers in one embodiment, to ameliorating the disease, disorder, or condition (i.e., slowing or arresting or reducing the development of the disease, disorder, or condition, or at least one of the clinical symptoms thereof). In another embodiment “treat”, “treating” or “treatment” refers to alleviating or ameliorating at least one physical parameter including those which may not be discernible by the subject. In yet another embodiment, “treat”, “treating” or “treatment” refers to modulating the disease, disorder, or condition, such as hypercytokinemia, either physically, (e.g., stabilization of a discernible symptom), physiologically, (e.g., stabilization of a physical parameter), or both. In yet another embodiment, “treat”, “treating” or “treatment” refers to prophylaxis (preventing or delaying the onset or development or progression of the disease, disorder, or condition, such as hypercytokinemia).

As used herein, the term “variant” in the context of a variant of a signal transducer CD24 protein means a peptide or polypeptide that differs in amino acid sequence by the insertion, deletion, or conservative substitution of amino acids, but retains at least one biological activity of the signal transducer CD24 protein. Likewise, as used herein, the term “fragment” or “portion” in the context of a fragment or portion of a signal transducer C24 protein means a sub-sequence less than the full-length sequence, but retaining at least one biological activity of the signal transducer CD24 protein. Representative examples of “biological activity” in the context of fragments and variants of a given CD24 molecule include the ability to do one or more of the following: binding molecules that exhibit DAMPs; participating in the formation of a trimolecular complex composed of the CD24 molecule, DAMPs, and Siglec (such as and Siglec G (mouse) or Signlec 10 (human) form); recruitment of Src family protein tyrosine kinases (PTKs) such as c-fgr, lyn and lck via membrane rafts; activating the mitogen-activated protein kinase pathway; and binding antibodies that specifically bind other CD24 molecules (Fang X et al., 2010).

The term variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol., 1982, 157:105-132. The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions may be performed with amino acids having hydrophilicity values within ±2 of each other. Both the hyrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

In some embodiments, the term “variant” in the context of CD24 refers to a sequence having at least 60%, 65%, 70%, 75%, 80%, 85%, 90%, 95%, 96%, 97%, 98%, or 99% sequence identity to the amino acid sequence of the human mature CD24 polypeptide SETTTGTSSNSSQSTSNSGLAPNPTNATTKAAG, or SETTTGTSSNSSQSTSNSGLAPNPTNATTK, or SETTTGTSSNSSQSTSNSGLAPNPTNATTK(V/A), and retains at least one biological activity of the CD24 protein.

In some embodiments, the term “variant” in the context of a CPP refers to a sequence having at least 60%, 65%, 70%, 75%, 80%, 85%, 90%, 95%, 96%, 97%, 98%, or 99% sequence identity to the amino acid sequence of a CPP disclosed herein, and retains its CPP transport function (i.e., membrane-traversing carrier function).

The term “variant” may also be used in the context of a genetic variant of a causative agent of inflammation, hyper-inflammation, or cytokine storm, such as a variant of a coronavirus (e.g., a variant of SARS-CoV-2).

As used herein, the term “vesicle” refers to a particle having an interior core surrounded and enclosed by a membrane comprising at least one lipid layer (e.g., at least one lipid monolayer or at least one lipid bilayer). The vesicle may be a cell-derived particle (an extracellular vesicle (EV)) having an interior core surrounded and enclosed by a membrane comprising at least one lipid layer (e.g., at least one lipid monolayer or at least one lipid bilayer), or may be a lipid vesicle (LV) having an interior core surrounded and enclosed by a membrane comprising at least one lipid layer (e.g., at least one lipid monolayer or at least one lipid bilayer). EVs are not cells and cannot replicate. EVs are typically unilamellar in structure, and may be spherical or have a non-spherical or irregular, heterogeneous shape. Some EVs have multiple layers of membranes. Examples of EVs include exosomes, microvesicles, mitovesicles, apoptotic bodies, microparticles, ectosomes, oncosomes, and many other names in the literature.

As used herein, the term “internalized by the vesicle” refers the binding agent described herein contained within the core of the vesicle. The entire binding complex or a portion thereof can be present in the core of the vesicle.

As used herein, the term “associated with the vesicle” refers the binding agent described herein that is bound by the outer surface of the vesicle and/or embedded within the vesicle membrane. The entire binding complex or a portion thereof can be associated with the vesicle.

### Aspects

Aspect 1. A method for loading a vesicle with a cargo molecule comprising CD24, or a biologically active fragment or variant of CD24, the method comprising contacting the vesicle with a binding complex, wherein the binding complex comprises the cargo molecule and a cell penetrating polypeptide (CPP) covalently or non-covalently coupled to the cargo molecule, and wherein the binding complex becomes internalized by the vesicle, associated with the vesicle, or a combination thereof to produce a loaded vesicle.

Aspect 2. The method of Aspect 1, wherein the vesicle includes a lumen surrounding by an outer layer having an outer surface, wherein the CD24, or a biologically active fragment or variant of CD24, includes extracellular amino acids, and wherein said extracellular amino acids become displayed on the outer surface of the vesicle after said contacting.

Aspect 3. The method of Aspect 2, wherein the CD24, or a biologically active fragment or variant of CD24, further includes transmembrane amino acids that become bound inside the outer layer and/or become presented in the lumen of the vesicle after said contacting.

Aspect 4. The method of any one of Aspects 1 to 3, wherein the vesicle is an extracellular vesicle (EV) or lipid vesicle (LV).

Aspect 5. The method of any one of Aspects 1 to 4, wherein the CPP is non-covalently coupled to the cargo molecule.

Aspect 6. The method of any one of Aspects 1 to 4, wherein the CPP is covalently coupled to the cargo molecule by a disulfide bond, an amide bond, a chemical bond formed between a sulfhydryl group and a maleimide group, a chemical bond formed between a primary amine group and an N-Hydroxysuccinimide (NHS) ester, a chemical bond formed via Click chemistry, or other covalent linkage.

Aspect 7. The method of 6, wherein the CPP is covalently coupled to the cargo molecule by a cleavable linker.

Aspect 8. The method of Aspect 7, wherein the cleavable linker is a photo-cleavable linker.

Aspect 9. The method of any one of Aspects 1 to 8, wherein the CPP is one listed in Table 2.

Aspect 10. The method of any one of Aspects 1 to 8, wherein the CPP is selected from among the following: Tat, Antennapedia, VP22, CaP, YopM, Artificial protein B1, 30Kc19, engineered +36 GFP, and naturally supercharged human protein.

Aspect 11. The method of any one of Aspects 1 to 8, the CPP is a peptide having from 4 to 40 amino acids, where the peptide includes from 4 to 40 arginine residues.

Aspect 12. The method of any one of Aspects 1 to 8, the CPP is a peptide having from 4 to 15 amino acids, where the peptide includes from 4 to 15 arginine residues.

Aspect 13. The method of any one of Aspects 1 to 8, the CPP is a peptide comprising 8 to 12 arginine residues.

Aspect 14. The method of any one of Aspects 1 to 8, the CPP is a peptide consisting of 8 to 12 arginine residues.

Aspect 15. The method of any one of Aspects 1 to 14, wherein the method further comprises the step of coupling CPP to the cargo molecule prior to contacting the vesicle with the binding complex.

Aspect 16. The method of any one of Aspects 1 to 15, wherein the CD24 or biologically active fragment or variant thereof is recombinantly produced.

Aspect 17. The method of any one of Aspects 1 to 15, wherein the CD24 or biologically active fragment or variant thereof is synthesized.

Aspect 18. The method of any one of Aspects 1 to 15, wherein the CD24 is human CD24.

Aspect 19. The method of any one of Aspects 1 to 18, wherein the cargo molecule includes a further molecule fused directly or indirectly to the CD24 or the biologically active fragment or variant thereof, wherein the further molecule is selected from among a small molecule (e.g., a drug, a fluorophore, a luminophore), macromolecule such as polyimide, proteins (e.g., enzymes, membrane-bound proteins), polypeptide (natural or modified), nucleic acid (e.g., natural, damaged or chemically modified DNA, DNA plasmid or vector, telomere, DNA quadruplex, DNAzyme, DNA-like molecule, antisense oligonucleotide, locked nucleic acid, threose nucleic acid, peptide nucleic acid (PNA), single or double-stranded nucleic acid, natural, damaged or chemically modified RNA, glycoRNA, enzymatic catalytic RNA, RNAzyme, ribozyme, non-coding RNA (ncRNA) such as microRNA (miRNA), small nuclear RNA (snRNA), interfering RNA such siRNA or shRNA, single guide RNA for a gene editing enzyme (e.g., Cas9), messenger RNA (mRNA), transfer RNA (tRNA), and ribosomal RNA (rRNA)), antibody or antibody-fragment, lipoprotein, carbohydrate, or glycoprotein.

Aspect 20. The method of any one of Aspects 1 to 19, wherein the vesicle is an extracellular vesicle (EV) and the EV is obtained from a mature cell.

Aspect 21. The method of any one of Aspects 1 to 19, wherein the vesicle is an extracellular vesicle (EV) and the EV is obtained from a stem cell or progenitor cell.

Aspect 22. The method of any one of Aspects 1 to 19, wherein the vesicle is an extracellular vesicle (EV) and the EV is obtained from a human or non-human animal mesenchymal stem cell.

Aspect 23. The method of any one of Aspects 1 to 19, wherein the vesicle is a lipid vesicle (LV) and the LV is a liposome.

Aspect 24. The method of any one of Aspects 1 to 19, wherein the vesicle is a lipid vesicle (LV) and the LV is a lipid nanoparticle, lipid droplet, micelle, reverse micelle, or lipid-polymer hybrid nanoparticle.

Aspect 25. The method of any one of Aspects 1 to 24, wherein the cargo molecule further comprises a detectable agent or medical imaging agent or is attached to a detectable or medical imaging agent, such as a fluorescent compound (e.g., a fluorophore) to serve as a marker, dye, tag, or reporter.

Aspect 26. The method of any one of Aspects 1 to 25, wherein the vesicle further comprises a targeting agent that targets the vesicle to a cell type, organ, or tissue (e.g., cancer cells, neural cells of the central nervous system or peripheral nervous system, or muscle cells).

Aspect 27. The method of any one of Aspects 1 to 26, wherein after the vesicle is loaded with the binding complex, the cargo molecule is uncoupled from the binding complex.

Aspect 28. The loaded vesicle produced by the method of any one of Aspects 1 to 27.

Aspect 29. A loaded vesicle comprising a binding complex, wherein the binding complex comprises a cargo molecule and a cell penetrating polypeptide (CPP) covalently or non-covalently coupled to the cargo molecule, wherein the binding complex becomes internalized by the vesicle, associated with the vesicle, or a combination thereof.

Aspect 30. The loaded vesicle of Aspect 29, wherein the loaded vesicle comprises a binding complex, wherein the binding complex comprises the cargo molecule and CPP covalently or non-covalently coupled to the cargo molecule.

Aspect 31. The loaded vesicle of Aspect 29 comprising an outer layer having an outer surface; a lumen surrounded by the outer layer; and a cargo molecule comprising CD24, or a biologically active fragment or variant of CD24, wherein the CD24, or biologically active fragment or variant includes extracellular amino acids displayed on the outer surface of the vesicle and, optionally, transmembrane amino acids are bound inside the outer layer and/or presented in the lumen of the vesicle.

Aspect 32. The loaded vesicle of Aspect 31, wherein the CPP is non-covalently coupled to the cargo molecule.

Aspect 33. The loaded vesicle of Aspect 31, wherein the CPP is covalently coupled to the cargo molecule by a disulfide bond, an amide bond, a chemical bond formed between a sulfhydryl group and a maleimide group, a chemical bond formed between a primary amine group and an N-Hydroxysuccinimide (NHS) ester, a chemical bond formed via Click chemistry, or other covalent linkage.

Aspect 34. The loaded vesicle of Aspect 33, wherein the CPP is coupled to the cargo molecule by a cleavable linker.

Aspect 35. The loaded vesicle of Aspect 34, wherein the cleavable linker is a photo-cleavable linker.

Aspect 36. The loaded vesicle of any one of Aspects 29 to 35, wherein the cargo molecule further comprises a molecule selected from among a small molecule (e.g., a drug, a fluorophore, a luminophore), macromolecule such as polyimide, proteins such as enzymes or membrane bound proteins, polypeptide (natural or modified), nucleic acid (e.g., natural, damaged or chemically modified DNA, DNA plasmid or vector, telomere, DNA quadruplex, DNAzyme, DNA-like molecule, antisense oligonucleotide, locked nucleic acid, threose nucleic acid, peptide nucleic acid (PNA), single or double-stranded nucleic acid, natural, damaged or chemically modified RNA, glycoRNA, catalytic RNA, RNAzyme, ribozyme, ncRNA (e.g., miRNA), small nuclear RNA (snRNA), interfering RNA such siRNA or shRNA, single guide RNA for a gene editing enzyme (e.g., Cas9), messenger RNA (mRNA), transfer RNA (tRNA), and ribosomal RNA (rRNA)), antibody or antibody-fragment, lipoprotein, carbohydrate, or glycoprotein.

Aspect 37. The loaded vesicle of any one of Aspects 29 to 36, wherein the vesicle is an extracellular vesicle (EV), wherein the EV is obtained from a mature cell.

Aspect 38. The loaded vesicle of any one of Aspects 29 to 36, wherein the vesicle is an extracellular vesicle (EV), and wherein the EV is obtained from a stem cell or progenitor cell.

Aspect 39. The loaded vesicle of any one of Aspects 29 to 36, wherein the vesicle is an extracellular vesicle (EV) and the EV is obtained from a human or non-human animal mesenchymal stem cell.

Aspect 40. The loaded vesicle of any one of Aspects 29 to 36, wherein the vesicle is a lipid vesicle (LV) and the LV is a liposome.

Aspect 41. The loaded vesicle of any one of Aspects 29 to 36, wherein the vesicle is a lipid vesicle (LV) and the LV is a lipid nanoparticle, lipid droplet, micelle, reverse micelle, or lipid-polymer hybrid nanoparticle.

Aspect 42. The loaded vesicle of any one of Aspects 29 to 41, wherein the cargo molecule further comprises a detectable agent or medical imaging agent, or is attached to a detectable or medical imaging agent, such as a fluorescent compound (e.g., a fluorophore) to serve as a marker, dye, tag, or reporter.

Aspect 43. The loaded vesicle of any one of Aspects 29 to 41, wherein the vesicle further comprises a targeting agent that targets the vesicle to a cell type, organ, or tissue (e.g., cancer cells, neural cells of the central nervous system or peripheral nervous system, or muscle cells).

Aspect 44. The loaded vesicle of any one of Aspects 29 to 43, wherein the CPP is one listed in Table 2.

Aspect 45. The loaded vesicle of any one of Aspects 29 to 43, wherein the CPP is selected from among the following: Tat, Antennapedia, VP22, CaP, YopM, Artificial protein B1, 30Kc19, engineered +36 GFP, and naturally supercharged human protein.

Aspect 46. The loaded vesicle of any one of Aspects 29 to 43, the CPP is a peptide having from 4 to 40 amino acids, where the peptide includes from 4 to 40 arginine residues.

Aspect 47. The loaded vesicle of any one of Aspects 29 to 43, the CPP is a peptide having from 4 to 15 amino acids, where the peptide includes from 4 to 15 arginine residues.

Aspect 48. The loaded vesicle of any one of Aspects 29 to 43, the CPP is a peptide comprising 8 to 12 arginine residues.

Aspect 49. The loaded vesicle of any one of Aspects 29 to 43, the CPP is a peptide consisting of 8 to 12 arginine residues.

Aspect 50. The loaded vesicle of any one of Aspects 29 to 49, wherein the cargo molecule is uncoupled from the binding complex.

Aspect 51. A method for delivering CD24, or a biologically active fragment or variant of CD24, into a cell in vitro or in vivo, comprising administering a loaded vesicle of any one of Aspects 29 to 50, wherein the loaded vesicle is internalized into the cell.

Aspect 52. The method of Aspect 51, wherein the loaded vesicle is administered to reduce an inflammatory response in the subject.

Aspect 53. The method of Aspect 51, wherein the loaded vesicle is administered to treat, prevent, or delay the onset of hyper-inflammation in the subject.

Aspect 54. The method of Aspect 51, wherein the loaded vesicle is administered to treat, prevent, or delay the onset of cytokine storm in the subject.

Aspect 55. The method of Aspect 54, wherein the cytokine storm is caused by an infectious agent (e.g., SARS-CoV-2 or a variant thereof).

Aspect 56. The method of Aspect 54, wherein the cytokine storm is caused by a monogenic disorder or autoimmune disorder (e.g., auto-inflammatory disorder, primary or secondary hemophagocytic lymphohistiocytosis (HLH)).

Aspect 57. The method of Aspect 54, wherein the cytokine storm has an iatrogenic cause (e.g., CAR-T cell therapy, blinatumomab, other T-cell engaging immunotherapy, or gene therapy).

Aspect 58. The method of Aspect 54, wherein the cytokine storm is caused by cancer, one or more cancer therapies (e.g., chemotherapy, immunotherapy, radiotherapy), or both.

Aspect 59. The method of any one of Aspects 51 to 58, wherein the method further comprises the step of loading the vesicle with the cargo molecule prior to administering the loaded vesicle to the cell.

Aspect 60. The method of any one of Aspects 51 to 59, wherein the method further comprises the step of coupling the CPP to the cargo molecule prior to contacting the vesicle with the binding complex.

Aspect 61. The method of Aspect 55, wherein the infectious agent is SARS-CoV-2 or a variant thereof, and wherein the method further comprises administering an antiviral drug, a monoclonal antibody treatment, a steroid, COVID-19 convalescent plasma, or a combination of two or more of the foregoing to the subject before, during, and/or after administration of the loaded vesicle.

### EXAMPLES

### Materials and Methods

Cell culture. Human primary dermal fibroblasts were purchased from ATTC (Cell Biology Collection), cultured in Dulbecco's modified Eagle's medium (DMEM) (Life Technologies, Carlsbad, Calif., USA) or fibroblast complete medium (PromoCell-C-23010). Fibroblasts were grown at 37° C. and under 5% CO₂ in cell culture flasks (BD falcon) as per manufacturer's instructions.

Exosome isolation and characterization. Human adipose-derived mesenchymal stem cell (MSC)-derived exosomes were purchased from EriVan Bio, LLC (Gainesville, Fla., USA). The particle diameter and concentration were assessed using NanoSightNS300 instrument (EriVan Bio, LLC, Gainesville, Fla., USA). The characterization of surface markers present in the exosomes was performed by EriVan Bio, LLC (Gainesville, Fla., USA). The exosomes were used in all assays described in Materials and Methods.

Cloning, expression, purification, and fluorescent labeling of GST-CD24-R9 and CD24-R9. The DNA sequence encoding human CD24 gene was fished out from UniProt. The codon optimized nucleotide sequence for the gene encoding GST-CD24-R9 (a cysteine residue was also added to the C-terminus of R9) was synthesized by GenScript and cloned into pET28C vector with Ndel-BamHI restriction sites through conventional cloning methods. The accuracy of the cloned gene sequence was checked through DNA sequencing. The recombinant protein GST-CD24-R9 was overexpressed in E. coli BL21(DE3) and purified by column chromatography. Furthermore, the GST tag was removed from GST-CD24-R9 to prepare the recombinant protein CD24-R9. Considering that the GST tag has four cysteine residues and the C-termini of GST-CD24-R9 and CD24-R9 are added one cysteine residue, GST-CD24-R9 or CD24-R9 was reacted with 24-fold molar excess of Cyanine3 maleimide or Cyanine5 maleimide for four hours at room temperature in order to covalently link Cyanine3 (Cy3) or Cyanine5 (Cy5) to the cysteine residues for fluorescent labeling by following the instructions of the manufacturer (Lumiprobe Corp., Hunt Valley, Md., USA). Any unreacted Cyanine3 maleimide or Cyanine5 maleimide was removed from the fluorescently labeled GST-CD24-R9 or CD24-R9 through a Bio-spin 6 column (Bio-Rad, Hercules, Calif., USA).

Loading of exosomes with CD24-R9. The purified recombinant protein GST-CD24-R9 or CD24-R9 (50 μg) in PBS was added to a solution of the exosomes (1×10¹¹ particles/mL) in phosphate-buffered saline (PBS) and the mixture was incubated for two hours at room temperature for cargo loading. The unattached GST-CD24-R9 or CD24-R9 was removed by first washing the exosomes with PBS for three times, concentrated the washed exosomes by using an Exosome Spin Column (MW 3000) (Invitrogen, Carlsbad, Calif., USA), and/or finally subjected the concentrated exosomes to filtration by using Amicon Ultra-centrifugal filters (100 K device, Merck Millipore, Billerica, Mass., USA).

Total Internal Reflection Fluorescence (TIRF) microscopy and image analysis. The exosomes in 35 mm μ-dish glass bottom culture dishes were initially incubated with either GST-CD24-R9 or CD24-R9 (50 μg/mL) for two hours at room temperature. The exosomes were then washed three times with PBS to remove any unattached proteins. After washing, the loaded exosomes were subjected to TIRF imaging measurements using Nikon Eclipse Ti microscope and the images were processed and analyzed by using ImageJ.

Internalization of the exosomes loaded with GST-CD24-R9 into human primary dermal fibroblast cells monitored by confocal microscopy and TIRF microscopy imaging. Human primary dermal fibroblast cells in a 35 mm μ-dish glass bottom culture dish was initially incubated with a culture medium containing the exosomes loaded with GST-CD24-R9 for 4 hours at 37° C. under 5% CO₂. The medium was then removed and the fibroblasts were washed for three times with PBS. The fibroblast cells were fixed with image-iT fixative solution (Invitrogen) as per manufactures protocol, and the nuclei counterstained with DAPI (Cell Biolabs). The fibroblasts were then subjected to confocal and TIRF microscopy imaging measurements.

Cell proliferation assay. Prior to the MTS assay, the fibroblasts were cultured onto 96-well culture plate at a cell density of 5×10⁴ cells/well. After 24 hr of incubation, the individual fibroblasts were supplemented with PBS (the control), CD24-R9, exosomes, or the exosomes loaded with CD24-R9. The exosome concentration in each case was 1×10⁸ particles/ml. At different time points (24, 48, and 72 h), cell proliferation was measured by following the manufacturer's protocol. In brief, 20 μl of MTS labelling reagent was added to each well and the plate was incubated at 37° C. for 1 hr. After incubation, the absorbance was read at 490 nm.

Cell invasion assay. The effects of the loaded or unloaded exosomes on fibroblast invasion were investigated using a CYTOSELECT™ 24-Well Cell Invasion Assay (Cell Biolabs, San Diego, Calif., USA) by following the manufacturer's instructions. Specifically, fibroblasts were seeded in serum-free medium containing PBS (the control), CD24-R9, exosomes, or the exosomes loaded with CD24-R9. Treated fibroblasts were added into the upper chambers of the assay system (1×10⁶ cells/well), whereas the bottom wells were filled with complete medium. Incubation was carried out for 48 h at 37° C. under 5% CO₂. The exosome concentration in each case was 1×10⁸ particles/ml. Subsequently, non-invasive fibroblasts in the upper chamber were removed from the upper inserts, and the cells that had invaded through the basement membrane were stained with cell stain solution provided in the kit for 10 min at room temperature. Subsequently, the stained cells were photographed under a brightfield microscope. Finally, the photographed inserts were transferred to an empty well filled with 200 μl extraction solution. After 10 min incubation on an orbital shaker, 100 μl of the samples were transferred to a 96 well microtiter plate for absorbance measurement at 560 nm by using a microplate reader (Spectramax iD5).

Statistical analysis. All the experiments were independently performed for at least four times. All data are means±SD. All statistical analysis and graphical representation were performed using GraphPad Prism or SigmaStat. The statistically significant differences were assessed by one-way and two-way ANOVA, and Tukey post hoc HSD tests. p values<0.05 were considered as statistically significant (*<0.05; **<0.01; ***<0.001).

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### Example 1—Cell-Penetrating Peptide R9 is Able to Carry and Load a Protein Cargo into Exosomes as Detected via TIRF Microscopy

TIRF microscopy was performed to assess the internalization of GST-CD24-R9, a fusion protein of the GST tag, human CD24, a CPP named R9 (amino acid sequence: RRRRRRRRR) (SEQ ID NO:1), and a C-terminal cysteine (Cys) residue. First, the purified recombinant protein GST-CD24-R9 was Cyanine3 (Cy3)-labeled at both its C-terminal Cys residue and the four different Cys residues of the GST tag (denoted as “GST-CD24-R9-Cy3”). Second, for cargo loading, the exosomes were simply mixed and incubated with the purified protein GST-CD24-R9-Cy3 for two hours at room temperature (see Materials and Methods section). Finally, the loaded exosomes were purified from any free GST-CD24-R9-Cy3 molecules before being evaluated using TIRF microscopy. At the Cy3 channel, bright fluorescence was detected from the exosomes (FIG. 1A), indicating that R9 was able to carry multiple copies of GST-CD24-R9-Cy3 into individual exosomes.

### Example 2—Cell-Penetrating Peptide R9 is Able to Carry and Load a Protein Cargo CD24 into Exosomes as Detected via Confocal Microscopy

In addition, confocal microscopy was performed to assess the internalization of CD24-R9, another fusion protein which was generated from the cleavage of the GST tag from GST-CD24-R9 by TEV protease (see Materials and Methods section). The purified CD24-R9 was chemically conjugated with Cyanine5 (Cy5) via its C-terminal Cys residue to produce CD24-R9-Cy5. For cargo loading, the exosomes were simply mixed and incubated with the purified CD24-R9-Cy5 for two hours at room temperature (see Materials and Methods section). After purification from free CD24-R9-Cy5 molecules, the exosomes loaded with CD24-R9-Cy5 were evaluated using confocal microscopy. At the Cy5 channel, bright fluorescence was detected from the exosomes (FIG. 2A), indicating that multiple copies of CD24-R9-Cy5 were loaded into individual exosomes.

### Example 3—Extracellular Amino Acid Residues of Human CD24 are Displayed on the Outside Surface of Exosomes While the Rest of the Residues of CD24 are Either Bound Inside the Membrane or Presented in the Lumen of the Exosomes

The Cy5-labeled anti-CD24 antibody and TIRF microscopy were used to assess the specific localization of GST-CD24-R9-Cy3 loaded onto/into the exosomes by two different approaches. In the first approach, the recombinant protein GST-CD24-R9-Cy3 was incubated with the Cy5-labeled anti-CD24 antibody for two hours to form a protein complex via the antigen/antibody interactions. Next, the purified exosomes were simply mixed and incubated with the protein complex for two hours at room temperature for cargo loading as described in the Materials and Methods section. In the second approach, the exosomes were first loaded with GST-CD24-R9-Cy3. The loaded exosomes were then incubated with the Cy5-labeled anti-CD24 antibody. For both approaches, the final exosomes after being washed off the unbound protein molecules were evaluated using TIRF microscopy. Notably, bright Cy5 fluorescence was observed from the second approach (FIG. 3C-1), indicating that the extracellular amino acid residues of human CD24 in many copies of GST-CD24-R9-Cy3, which were bound onto the membrane of the exosomes, were located on the outside surface of the exosomes and thereby bound by the Cy5-labeled anti-CD24 antibody as detected at the Cy5 channel. Each GST-CD24-R9-Cy3 molecule was likely anchored onto the exosome membrane via the C-terminal hydrophobic residues of the CD24. These conclusions were supported by similarly bright Cy5 fluorescence observed from the loaded exosomes by the first approach (FIG. 3A-1). Furthermore, the loading of GST-CD24-R9-Cy3 into the exosomes was also indicated by the bright Cy3 fluorescent signals from both approaches (FIGS. 3B-1 and 3D-1).

### Example 4—Time-Dependent Loading of CD24 into Exosomes

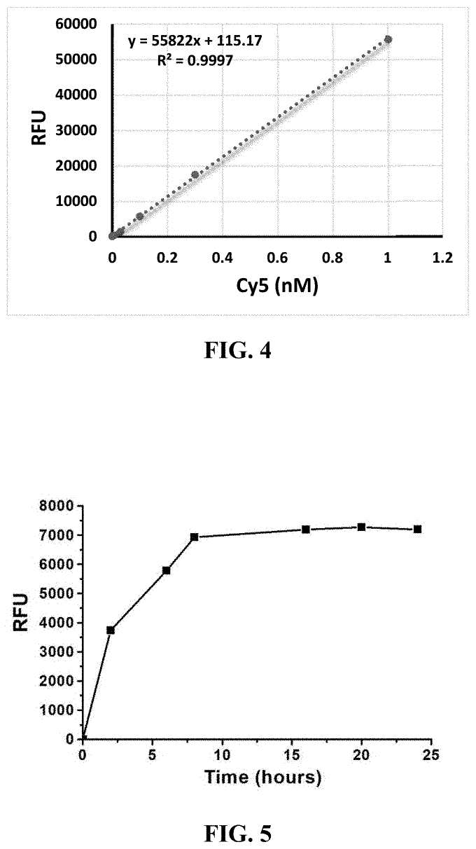

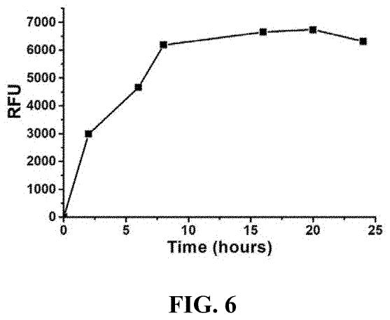

The quantity of CD24-R9-Cy5 in loaded exosomes was determined by comparing its Cy5 fluorescence intensity level with that of Cy5 standard curve. The purified CD24-R9-Cy5 (50 μg) in PBS was added to a solution of exosomes (1×10⁸ particles/mL) in PBS and the mixture was incubated for 0, 2, 6, 8, 16, 20, or 24 hours at room temperature. The unattached CD24-R9-Cy5 was removed by washing with PBS for three times and filtration using Amicon Ultra-centrifugal filters (100 K device, Merck Millipore, Billerica, Mass., USA). The Cy5 fluorescence was measured in 100 ul samples at room temperature in a SpectraMax iD5 Multimode Microplate Reader with 646/678 nm filter. The concentration of the exosomes loaded with CD24-R9-Cy5 was determined from the Cy5 standard curve (FIG. 4). The maximum loading capacity was observed at 8 hours of incubation of CD24-R9-Cy5 with the exosomes (FIG. 5). The concentration of CD24-R9-Cy5 loaded into the exosomes was then determined to be 0.1221 μg/ml, which corresponds to 60,230 loaded protein molecules per exosome.

### Example 5—Time-Dependent Loading of GST-CD24-R9 onto the Membrane of Exosomes

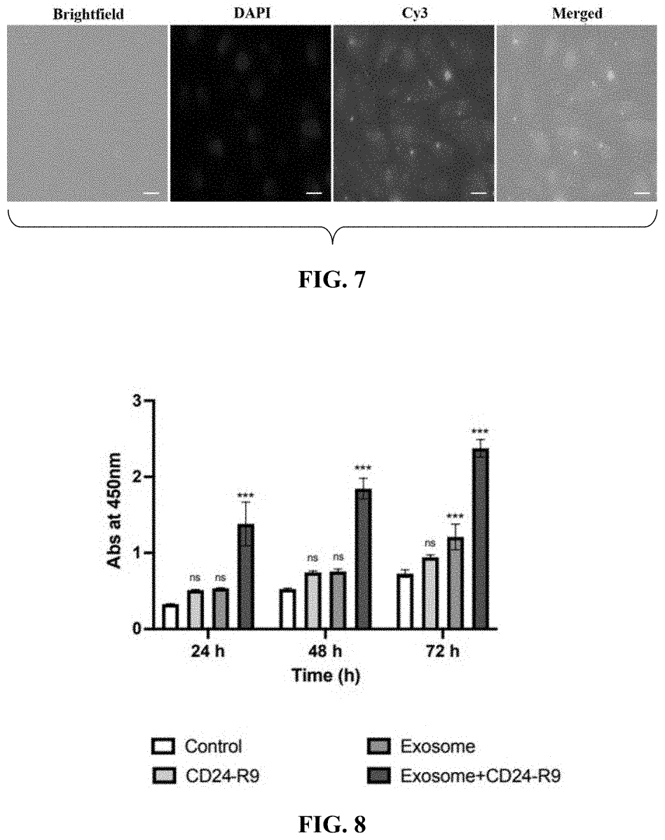

The quantity of GST-CD24-R9 on the surface of the exosomes was determined by comparing its fluorescence reading with that of Cy5 standard curve. The purified GST-CD24-R9-Cy3 (50 μg, 38.3 kDa) in PBS was added to a solution of exosomes (1×10⁸ particles/mL) in PBS and the mixture was incubated for 0, 2, 6, 8, 16, 20, 24 hours at room temperature. The unattached GST-CD24-R9-Cy3 was removed by washing with PBS for three times and then filtration using Amicon Ultra-centrifugal filters (100 K device, Merck Millipore, Billerica, Mass., USA). After washing, the loaded exosome at different time points was subsequently incubated with the Cy5-labeled anti-CD24 antibody for 2 hours. The unattached anti-CD24 antibody was removed by washing the exosome/antibody complexes with PBS for three times and filtration using Amicon Ultra-centrifugal filters (100 K device, Merck Millipore, Billerica, Mass., USA). The Cy5 fluorescence of the antibody-bound exosomes was measured in 100 ul samples at room temperature in a SpectraMax iD5 Multimode Microplate Reader with 646/678 nm filter. The membrane-bound GST-CD24-R9 concentration was inferred and determined from the standard curve using the Cy5 standard curve (FIG. 4) as the Cy5-labeled anti-CD24 antibody binds to GST-CD24-R9 on the surface of the exosomes in a 1:1 molar ratio. The maximum loading capacity was observed at 8 hours of incubation of CD24-Cy5 antibody (FIG. 6). The maximum concentration of GST-CD24-R9 loaded onto the surface of the exosomes was determined to be 0.108 μg/ml, which corresponds to 1.75×10⁴ membrane-bound protein molecules per exosome. If the exosomes loaded with membrane-bound CD24 can be administered as a therapeutic or prophylactic treatment, they will be much more potent than natural exosomes which likely contain either none or a small number of copies of membrane-bound CD24 per exosome.

### Example 6—Exosomes Loaded with GST-CD24-R9-Cy3 Can Fuse with and Deliver the Protein Cargo into Human Cells

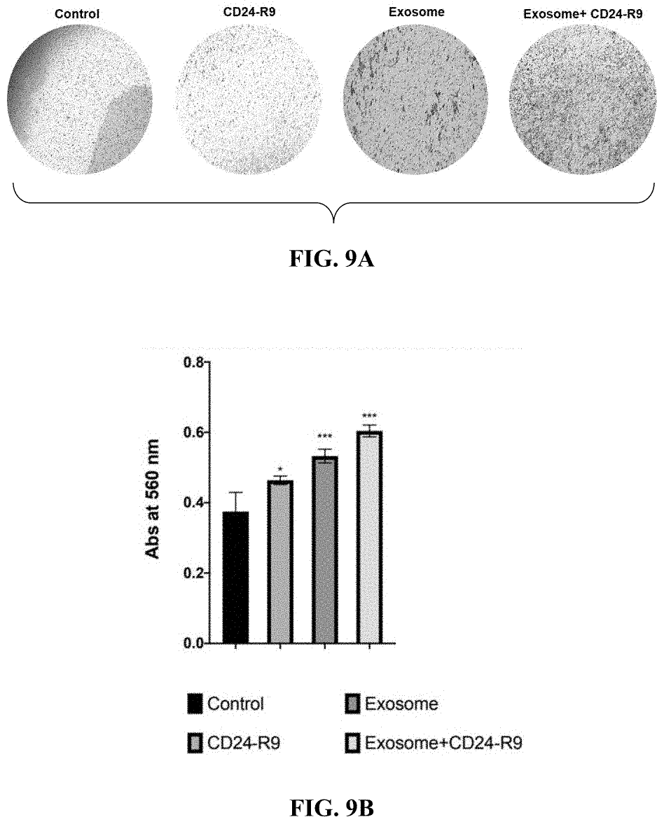

Human primary dermal fibroblast cells in a 35 mm μ-dish glass bottom culture dish were first incubated with a culture medium containing the exosomes loaded with the fusion protein GST-CD24-R9-Cy3 for 4 hours at 37° C. under 5% CO₂. The medium was then removed and the fibroblasts were washed for three times with PBS. The fibroblast cells were then fixed with image-iT fixative solution and the nuclei were counterstained with DAPI (see Materials and Methods section). The fibroblasts were then subjected to confocal microscopy imaging measurements. The strong Cy3 fluorescence signals and quite a few intense spots were observed in the cytoplasm, around and inside the nuclei of individual fibroblast cell (FIG. 7), indicating that the loaded exosomes were fused with human fibroblast cells and multiple copies of the protein cargo GST-CD24-R9-Cy3 were loaded into individual cells. Thus, using the exosomes loaded with a protein cargo covalently coupled with a CPP (the cargo can be located in the membrane and lumen of the exosomes) is an efficient way to deliver the protein cargo into mammalian cells.

### Example 7—Exosomes Loaded with CD24-R9 Promote Cell Proliferation

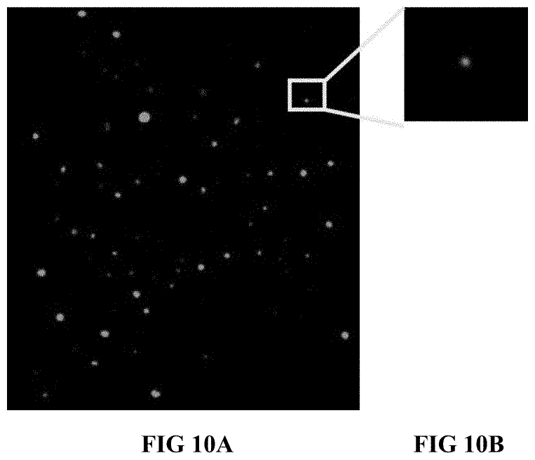

Fibroblast proliferation is important in tissue repair as fibroblasts are mainly involved in proliferation, migration, contraction, and collagen production leading to the formation of granulation tissue. Accordingly, cell proliferation assays were performed to investigate the effects of the human adipose-derived MSC-secreted exosomes loaded with CD24-R9 on the proliferation of human primary dermal fibroblasts using a colorimetric MTS proliferation assay kit (see Material and Methods section). The treatment of human primary dermal fibroblasts with the exosomes loaded with CD24-R9 for 24, 48, and 72 hours enhanced fibroblast proliferation by 3.3-4.2 fold relative to the PBS (“control”), 2.5-2.7 fold relative to the recombinant protein CD24-R9, 2.0-2.6 fold relative to the unloaded exosomes (Table 3 and FIG. 8). Thus, both the cargo protein CD24 and the exosomes boosted fibroblast proliferation.

TABLE 3
Proliferation rate enhancement of human primary dermal
fibroblasts treated with the exosomes loaded with CD24-R9
(“Exosome + CD24-R9”) relative to other treatments.

| | 24 hours | 48 hours | 72 hours |
|---|---|---|---|
| “Exosome + CD24-R9” | 4.2-fold | 3.5-fold | 3.3-fold |
| “the control” | | | |
| “Exosome + CD24-R9” | 2.7-fold | 2.5-fold | 2.5-fold |
| “CD24-R9” | | | |
| “Exosome + CD24-R9” | 2.6-fold | 2.4-fold | 2.0-fold |
| “Exosome” | | | |

### Example 8—Exosomes Loaded with CD24-R9 Enhanced Cell Invasion

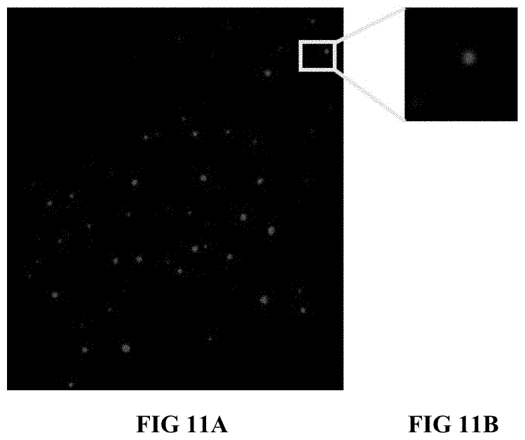

Cell invasion assays were performed to investigate the effect of the exosomes loaded with CD24-R9 on the invasion of human primary dermal fibroblasts using a colorimetric transwell invasion assay kit (see Material and Methods section). As shown in FIGS. 9A-9B, treatment with the human adipose-derived MSC-secreted exosomes loaded with CD24-R9 for 48 hours enhanced the invasion of human primary dermal fibroblasts by 1.3-fold compared to the treatment with the recombinant protein CD24-R9, 1.1-fold compared to the treatment with the unloaded exosomes, and 1.6-fold compared to the treatment with PBS (control) (p<0.001).

### Example 9—Cell-Penetrating Ppeptide R9 is Able to Carry and Load a Protein Cargo GST-CD24 into Liposomes as Monitored by TIRF Microscopy

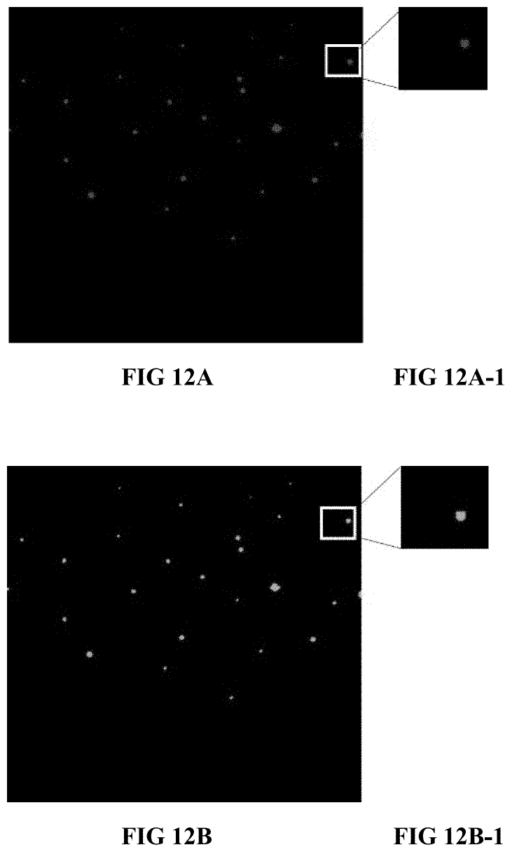

TIRF microscopy was performed to assess the internalization of GST-CD24-R9 which is a fusion protein cargo between GST-CD24 and the CPP peptide R9. First, the purified recombinant protein GST-CD24-R9 containing a C-terminal cysteine residue was chemically conjugated with Cyanine3 (Cy3) to form GST-CD24-R9-Cy3 (GST has four cysteine residues which can also be conjugated with Cy3). For the loading of GST-CD24-R9-Cy3, the liposomes were simply mixed and incubated with the purified GST-CD24-R9-Cy3 for five hours at room temperature, then washed with PBS buffer for three times, and finally filtered using Amicon Ultra-centrifugal filters (100 K device, Merck Millipore, Billerica, Mass., USA). The loaded liposomes were evaluated using TIRF microscopy at the Cy3 channel. The loaded liposomes emitted bright Cy3 fluorescence (FIGS. 10A and 10B), indicating that the R9 peptide can load multiple copies of the protein cargo GST-CD24-R9-Cy3 into individual liposomes.

### Example 10—Cell-Penetrating Peptide R9 is Able to Carry and Load a Protein Cargo CD24 into Liposomes as Monitored by Confocal Microscopy

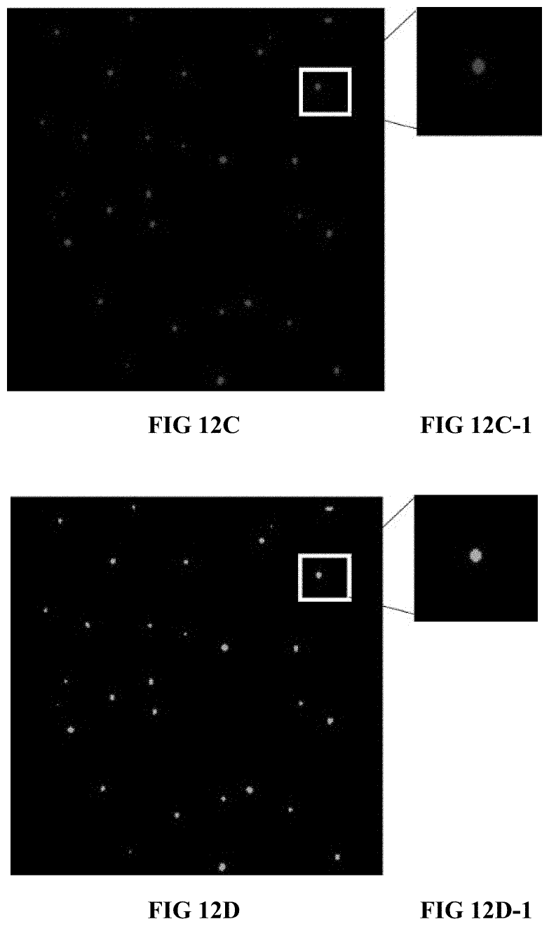

Similarly, confocal microscopy was performed to assess the internalization of CD24-R9, a fusion protein between CD24 and the R9 peptide. First, the recombinant protein CD24-R9 containing a C-terminal cysteine residue was chemically conjugated with a C-terminal Cyanine5 (Cy5) to form CD24-R9-Cy5. For loading, the liposomes were simply mixed and incubated with the purified protein CD24-R9-Cy5 for five hours at room temperature, then washed with PBS buffer for three times, and finally filtered using Amicon Ultra-centrifugal filters (100 K device, Merck Millipore, Billerica, Mass., USA). The loaded liposomes were evaluated using confocal microscopy at the Cy5 channel. Bright Cy5 fluorescence was observed from the loaded liposomes (FIGS. 11A and 11B), indicating that the R9 peptide can load multiple copies of CD24-R9-Cy5 into individual liposomes.

### Example 11—Cell-Penetrating Peptide R9 is Able to Carry and Load a Protein Cargo GST-CD24 onto the Surface of Liposomes as Monitored by TIRF Microscopy

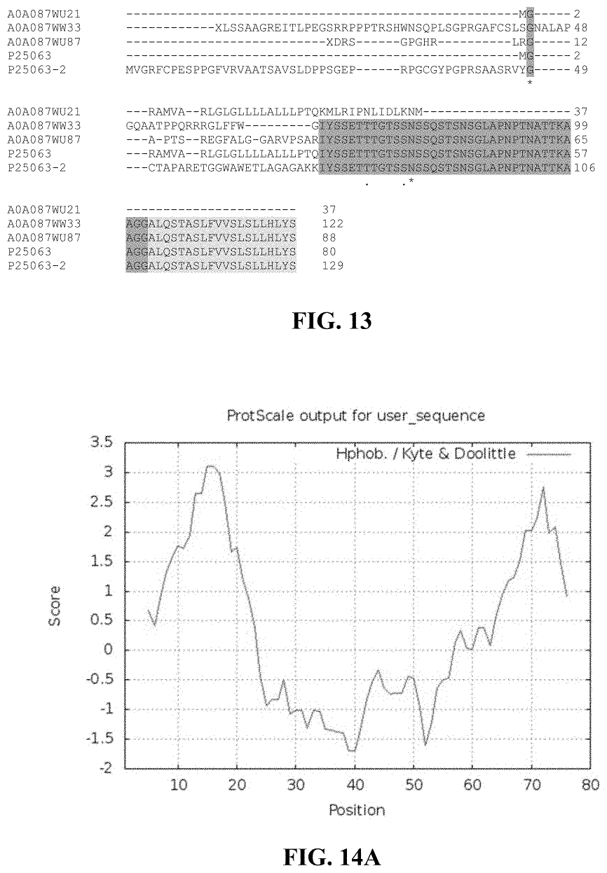

To accurately define the localization of the protein cargo GST-CD24-R9-Cy3 within liposomes, we used Cy5-labeled anti-CD24 antibody and TIRF microscopy. First, the recombinant and purified protein cargo GST-CD24-R9-Cy3 was incubated with the Cy5-labeled anti-CD24 antibody at room temperature for two hours to form a protein complex (the binding complex). For loading, the protein complex cargo was simply mixed and incubated with liposomes for five hours at room temperature, then washed with PBS buffer for three times, and finally filtered using Amicon Ultra-centrifugal filters (100 K device, Merck Millipore, Billerica, Mass., USA). In parallel, we first loaded liposomes with GST-CD24-R9-Cy3 for five hours at room temperature. After washing out unincorporated GST-CD24-R9-Cy3 using PBS buffer, the liposomes were incubated with the Cy5-labeled anti-CD24 antibody for two hours at room temperature. After washing out any unbound proteins for three times by using PBS buffer, the liposomes were filtered using Amicon Ultra-centrifugal filters (100 K device, Merck Millipore, Billerica, Mass., USA). The loaded liposomes from above two ways were evaluated using TIRF microscopy. Bright Cy5 fluorescence was observed from the liposomes at the Cy5 channel (FIGS. 12A, 12C), indicating that multiple copies of the loaded GST-CD24-R9-Cy3 protein were bound and located at the surface of individual liposomes because they bound to the free Cy5-labeled anti-CD24 antibody in the solution. In addition, bright Cy3 fluorescence (FIGS. 12B, 12D) was observed with the same loaded liposomes in (FIG. 12A) and (FIG. 12C) at the Cy3 channel, suggesting that multiple copies of GST-CD24-R9-Cy3 were loaded onto both the surface of and inside individual liposomes.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

1. A method for loading a vesicle with a cargo molecule comprising CD24, or a biologically active fragment or variant of CD24, the method comprising contacting the vesicle with a binding complex, wherein the binding complex comprises the cargo molecule and a cell penetrating polypeptide (CPP) covalently or non-covalently coupled to the cargo molecule, and wherein the binding complex becomes internalized by the vesicle, associated with the vesicle, or a combination thereof to produce a loaded vesicle.

2. The method of claim 1, wherein the vesicle includes a lumen surrounding by an outer layer having an outer surface, wherein the CD24, or a biologically active fragment or variant of CD24, includes extracellular amino acids, and wherein said extracellular amino acids become displayed on the outer surface of the vesicle after said contacting.

3. The method of claim 2, wherein the CD24, or a biologically active fragment or variant of CD24, further includes transmembrane amino acids that become bound inside the outer layer and/or become presented in the lumen of the vesicle after said contacting.

4. The method of claim 1, wherein the vesicle is an extracellular vesicle (EV) or lipid vesicle (LV).

5. The method of claim 1, wherein the CPP is non-covalently coupled to the cargo molecule.

6. The method of claim 1, wherein the CPP is covalently coupled to the cargo molecule by a disulfide bond, an amide bond, a chemical bond formed between a sulfhydryl group and a maleimide group, a chemical bond formed between a primary amine group and an N-Hydroxysuccinimide (NETS) ester, a chemical bond formed via Click chemistry, or other covalent linkage.

7. The method of 6, wherein the CPP is covalently coupled to the cargo molecule by a cleavable linker.

8. The method of claim 1, wherein the CPP is one listed in Table 2.

9. The method of claim 1, wherein the CPP is selected from among the following: Tat, Antennapedia, VP22, CaP, YopM, Artificial protein B1, 30Kc19, engineered +36 GFP, and naturally supercharged human protein.

10. The method of claim 1, the CPP is a peptide having from 4 to 40 amino acids, where the peptide includes from 4 to 40 arginine residues.

11. The method of claim 1, the CPP is a peptide comprising 8 to 12 arginine residues.

12. The method of claim 1, the CPP is a peptide consisting of 8 to 12 arginine residues.

13. The method of claim 1, wherein the CD24 or biologically active fragment or variant thereof is recombinantly produced or synthesized.

14. The method of claim 1, wherein the cargo molecule includes a further molecule fused directly or indirectly to the CD24 or the biologically active fragment or variant thereof, wherein the further molecule is selected from among a small molecule, a polyimide, a protein, a polypeptide, a nucleic acid, an antibody or antibody-fragment, a lipoprotein, a carbohydrate, or a glycoprotein.

15. The method of claim 1, wherein the cargo molecule further comprises a detectable agent or medical imaging agent or is attached to a detectable or medical imaging agent, such as a fluorescent compound to serve as a marker, dye, tag, or reporter.

16. The method of claim 1, wherein the vesicle further comprises a targeting agent that targets the vesicle to a cell type, organ, or tissue.

17. The method of claim 1, wherein after the vesicle is loaded with the binding complex, the cargo molecule is uncoupled from the binding complex.

18. A loaded vesicle produced by the method of claim 1.

19. A loaded vesicle comprising a binding complex, wherein the binding complex comprises a cargo molecule and a cell penetrating polypeptide (CPP) covalently or non-covalently coupled to the cargo molecule, wherein the binding complex becomes internalized by the vesicle, associated with the vesicle, or a combination thereof.

20. A method for delivering CD24, or a biologically active fragment or variant of CD24, into a cell in vitro or in vivo, comprising administering a loaded vesicle of claim 1, wherein the loaded vesicle is internalized into the cell.


SEQUENCE LISTING (28 sequences)

SEQ ID NO: 1 - protein, 646 aa, Homo sapiens
MGLPRLVCAFLLAACCCCPRVAGVPGEAEQPAPELVEVEVGSTALLKCGLSQSQGNLSHVDWFSVHKEKRTLIFRVRQGQGQSEPGEYEQRLSLQDRGATLALTQVTPQDERIFLCQGKRPRSQEYRIQLRVYKAPEEPNIQVNPLGIPVNSKEPEEVATCVGRNGYPIPQVIWYKNGRPLKEEKNRVHIQSSQTVESSGLYTLQSILKAQLVKEDKDAQFYCELNYRLPSGNHMKESREVTVPVFYPTEKVWLEVEPVGMLKEGDRVEIRCLADGNPPPHFSISKQNPSTREAEEETTNDNGVLVLEPARKEHSGRYECQGLDLDTMISLLSEPQELLVNYVSDVRVSPAAPERQEGSSLTLTCEAESSQDLEFQWLREETGQVLERGPVLQLHDLKREAGGGYRCVASVPSIPGLNRTQLVNVAIFGPPWMAFKERKVWVKENMVLNLSCEASGHPRPTISWNVNGTASEQDQDPQRVLSTLNVLVTPELLETGVECTASNDLGKNTSILFLELVNLTTLTPDSNTTTGLSTSTASPHTRANSTSTERKLPEPESRGVVIVAVIVCILVLAVLGAVLYFLYKKGKLPCRRSGKQEITLPPSRKSELVVEVKSDKLPEEMGLLQGSSGDKRAPGDQGEKYIDLRH

SEQ ID NO: 2 - protein, 220 aa, Homo sapiens
MPLTGVGPGSCRRQIIRALCLLLLLLHAGSAKNIWKRALPARLAEKSRAEEAGAPGGPRQPRADRCPPPPRTLPPGACQAARCQADSECPRHRRCCYNGCAYACLEAVPPPPVLDWLVQPKPRWLGGNGWLLDGPEEVLQAEACSTTEDGAEPLLCPSGYECHILSPGDVAEGIPNRGQCVKQRRQADGRILRHKLYKEYPEGDSKNVAEPGRGQQKHFQ

SEQ ID NO: 3 - protein, 195 aa, Homo sapiens
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKYQPPSTNKNTKSQRRKGWPKTHPGGEQKEGTEASLQIRGKKKEQRREIGSRNAECRGKKGK

SEQ ID NO: 4 - protein, 325 aa, Homo sapiens
MLPRVGCPALPLPPPPLLPLLLLLLGASGGGGGARAEVLFRCPPCTPERLAACGPPPVAPPAAVAAVAGGARMPCAELVREPGCGCCSVCARLEGEACGVYTPRCGQGLRCYPHPGSELPLQALVMGEGTCEKRRDAEYGASPEQVADNGDDHSEGGLVENHVDSTMNMLGGGGSAGRKPLKSGMKELAVFREKVTEQHRQMGKGGKHHLGLEEPKKLRPPPARTPCQQELDQVLERISTMRLPDERGPLEHLYSLHIPNCDKHGLYNLKQCKMSLNGQRGECWCVNPNTGKLIQGAPTIRGDPECHLFYNEQQEARGVHTQRMQ

SEQ ID NO: 5 - protein, 639 aa, Homo sapiens
MEDSQDLNEQSVKKTCTESDVSQSQNSRSMEMQDLASPHTLVGGGDTPGSSKLEKSNLSSTSVTTNGTGGENMTVLNTADWLLSCNTPSSATMSLLAVKTEPLNSSETTATTGDGALDTFTGSVITSSGYSPRSAHQYSPQLYPSKPYPHILSTPAAQTMSAYAGQTQYSGMQQPAVYTAYSQTGQPYSLPTYDLGVMLPAIKTESGLSQTQSPLQSGCLSYSPGFSTPQPGQTPYSYQMPGSSFAPSSTIYANNSVSNSTNFSGSQQDYPSYTAFGQNQYAQYYSASTYGAYMTSNNTADGTPSSTSTYQLQESLPGLTNQPGEFDTMQSPSTPIKDLDERTCRSSGSKSRGRGRKNNPSPPPDSDLERVFVWDLDETIIVFHSLLTGSYAQKYGKDPPMAVTLGLRMEEMIFNLADTHLFFNDLEECDQVHIDDVSSDDNGQDLSTYSFATDGFHAAASSANLCLPTGVRGGVDWMRKLAFRYRRVKELYNTYKNNVGGLLGPAKRDAWLQLRAEIEGLTDSWLTNALKSLSIISTRSNCINVLVTTTQLIPALAKVLLYSLGGAFPIENIYSATKIGKESCFERIMQRFGRKVVYVVIGDGVEEEQAAKKHNMPFWRISSHSDLLALHQALELEYL

SEQ ID NO: 6 - protein, 236 aa, Homo sapiens
MHTTQKDTTYTKIFVGGLPYHTTDASLRKYFEVFGEIEEAVVITDRQTGKSRGYGFVTMADRAAAERACKDPNPIIDGRKANVNLAYLGAKPRIMQPGFAFGVQQLHPALIQRPFGIPAHYVYPQAFVQPGVVIPHVQPTAAAASTTPYIDYTGAAYAQYSAAAAAAAAAAAYDQYPYAASPAAAGYVTAGGYGYAVQQPITAAAPGTAAAAAAAAAAAAAFGQYQPQQLQTDRMQ

SEQ ID NO: 7 - DNA, 14193 bp, Homo sapiens
  source 1..14193: mol_type = other DNA
ctgcctcagcctcccgagtagctgggactacaggcgcgcgcgaccacgcccagctaatttttgtatttttagtagagatggggtttcaccatgttggccaggatggtctcgatttcttgacctcgtgatccgcccgcctcggcctcccagagtgctgggattacaggcataaaccaccgcgcccagcctctttcctcccctctctactcccggctccaagatcagccctggcagcagcagtgagggtcccaggcatcgccgggatgggatggaacaaggctccccttctccagggctacccctgggatgctggcagccccgtgtggcagggaagaggaactgcaggagcgtccttcagggaggagaggggccagaagacctcaacatggcgcccccaggtttcccacaggccccctcctgtagaccagccggccttcccctcagtgacgactcaaagtcagatttgaagagctctctggggacattcctctccctggatggtggtattctggggagcggatgtgaacttccctgaaggggagaggtgtaaactcccagcatcctgctaccaccctccgcccccatcacccttcattccccaggtgcaggctgggggcagaggagagggaaggggattaggtgctggggccggggaaccgagacccagggtgtcaccgctgacagcagcggggcccgcagagctgcagggagacagagccgcctccccgcggcgccgacgctgccatctagctagagggccccactctttctttaaccagaggtcacagcctcattgcctacccaggaggcagccccgagacgccccctgcggcctgggttttctccaccctgtccccgcgcccaccccagctctgccatccccggctgcaccgctttgtaaaggggacggctggtcagaccgaatcactgcgcgggctcgcgctgggtcgggcctggagactggatctggggcttccggagagccccaggctccccgcgcgcccatccacccgcagccgcctgagcatttatggtaatgtttgggttggggcgagcggccggatccccgccccctgcctccgccgggcgggacctcgaggccggctcctggggagcttagcgccggcgccttcccgccgcggccgggtcccctgggtggaggccctgggtgccggaggcctgaggtccggcaggtgggctggcccccgtgcggaggctcctaagaccggggggtacgagagggacagtcccagtcggtcggaggaggggtccgcggggttggggactccgatcccgctcaacctaccctaaccacccactgctgcccacccaacccccgcctctggcttaagagtccatcatccccaggaggagtcccgatgcatccacatcctctttctccttctctccccccaccccctggcagggaaggggctgagggaagaggccagcagctctcaggggagtcgtcggaatgctgggagcctgggaacagaccctgcggtccctgggaaagccaggctgtgggaatcccaccgtcagccccctccccaggatgggccaccggatgtcagagaccccagttccccaacggggatctttgtgagggtgggccctgccccccagttcaagccccagcctggctcgaccccagagttcagtaggccatctgtgacgaagacagcccctcccccgctctccgccctccacccccctgcctgctccaacataatcctcgccattgtgcccgtcgccccctccccccgctccccctctctagacccctgtacaatggggcctttgcagcctccagaatggagatgaactcacggtttttccaagtttaaaaatagcccctcccatcccgcagaaggccaccctccctctctcccatggtggtgtggggtgcacctccatcccggctgtgcctccttcctcctgccctgggtcaggtgcgaaggacatgggtgagacagcatccggcgggagcagggctgcttcccccgattcaatctcttcccccacatcttccatccaccccccagtacagtatccggtttcctgccccctacccttttccaccctgttttcactccttcagtataatcttctcctactcacacccacattctggaaaaattctccaacttccgcaccccctccaaagacccaggccggatcctattatctggcctggccagggggttccccagtctcggtaactgccaagattgttgggactcccaccccatgtcctcacagcagctgcaaacacctctttatcagcttccttcctcgagctacttctgacccacccttctcatatcctcacatcctggtcaaggcccctggatccagaagggtgggtcagaagtcgctaagttccctttgctggagctgagagaggacacttgggttccagtcttgccccttcatggcctctggggacaggtccatccttgtctttttacctccaactccccagggttccacttctccttacaatgagctgaaagctccacccatccaggagactgtggctgaggagcccaaccaggacccccaccccacctggcctcagtttctccacctgtggtcagacctataccagagacacgcagagggcattatgcgcaggagggtcccctcatgtgtgaggggcccaggtccaccatggggctggaggaaagcagccttcttgcctgcaacccaacgagggaaaaaagaagaattaggactctgcccccagggcctcttctggccctggcctacatctgagccaggctcttccaacccagtccaggtgctgggtccctgcagggtagacggccgtggcctcctcctccaactcccttctgctggctgtcactcctgtcctgctggaagtcccgcagccaccttctctcccctggggctgccggagcactagcctctggcctctaagaagagctgagggctgatccccttcaccttccgctccctccaccctgctccctgcctccactcctgcgtcctttccgaggtcctgacccgtctttagtttggaatcccaaagtgcccacctcatgaaaccatcccaaagctatctgtcccctgaggctatgacccacttccgagcagcctagacagccccaggctactggcttccttaaaggacagtcttctgtgcttgtttggtggggcaatgcgaagggacgtcaccctgggctctaggctctgacctcatttccctttcaccgcagagacaaatcctccacccccacctggcttcactctcctccagcggaagaagaattcagaactgactgtccttggtggttagctggctttgttttcaggccagagaggcgaatgattagttaatggtgttaagtgaaggaagggggttgggcagggaagccagctccatgcccagccgggcctgccagggcaaacggcatccttcagccattcccactccagcccagcagacaaaacattctcatctagtcagctctgggcctggggccagtccatacattcactctggagggcgagccagcgtcctcctcttgttctccctgcaggctccaggctccaggcccctggcccggtgggcctgggctctgcacagcctctggctggagactaatggcattggaggaggggtcgagaaggctctgaaagacaagcctggtgtggttttccagtcatctctgcgtcatcaaaacgccaccccggagcatgctctcaagtcacctggccatctccagggtttgctggctcaccctccacctcccccagcgagcaaaggggtggccccgctgggcaacagacctctacccaaatccaggcccctatttggcactccctggtggaggcgcgccagcagatgtggaccccttttctgaggccaggctgctggggtcagagtgaggcaagagcacctccccctccctagagccttagttcaagaggccagaagacccagggagaagccggcgttttgggagaaggggagctgcagagctccccacccaagcaggtctgatttgattcttccctcagctatggacccacaatcaatagggctggggaagacatggggtgggggaagatcagcgctgccgggatcctttcaatcatcaggaacagcaacaggtttgcagggtcaggctggggaccctcgcccattaactctttcttctccctgtttctttctcttaggtgaggggaaactgagttccagggtaggctccagagtgaagagggaagaaacatgattctcaaggcccaggtctggacaagtgtgaacaccttgggcctgcgaattcagccccctccttcctttctctggtcaaaggctagacttgcaggagcttgcgtttgaagggacagcccagaaggcatcgtctgcactccccatacaggtacttctgggtctgtgggactggcgcagggttcttctcccaaagctgccagcactgaggctgaggcagtgtcaggccggcggcagcggcagtggtgcaatcgttctgggaagcgatagaggccggcctgaattctctgtggcaagggaggggagcccaagtgggaggccccttggggacaccgaggaccaggtccgctactgctcctcccccaggaggtcccctaggggctacattggctggcaggggctgagcagcggtgagcctggctggcttcgacccggggcgactccgggcatccgggacagcttctcctcgctgccacctcggccagtcagaccccgagacacctgtcactaccccctcagccttcccaagccaggagcctgggagtccggctctggcctacctccggcagcgctcctaggcgcagctcccgggctggcggcgccggggcccgccccctagggctgcggcgcgcggggcgggggctgggggctgcgcggggcggggcgggcccgggcgctccgggccccctcccccgcccccctgacgtcagcccccggcagcctcgagctgctcacttggctctcgccctccggccgggaagcatggggcttcccaggctggtctgcgccttcttgctcgccgcctgctgctgctgtcctcgcgtcgcgggtgagttcgctcgcaggggggccgcgccccggctaggggtctgcggtggagcgtgccagggagcagagccagcggcgcggcggggtcggggcgttgcgtctgggaggacgagcctcctccctgggtccccgatccccgggcgccttgcgcgcgagcaactcttctttgcagccagtttgcagccgggattctagagtatcccgggagcagcactcggaaggcggggaggaggctgcttctgggaacgagaaggggtggagctcagcctttcggggtgctggggggtgggtggtccctgaggtgctcactctgggggcccgcaattgaagccgggcaggaggcgcagctggggcgcatcctcaaagcctgaattccgcgcccgcctgttgctggaaaaggcagcttccttcgctggagggggtgcgccgacccaccccttcccccttctgcctgggcatcacgccaggctggaggtgagcgagagcgggaggttcggcggctcccgcccgagctgggcgttggcaggggttgcggggcggtgtgggtcgcctcgcgcctccccgagtgatgggatcataggggacagagatgagggatggaggattcccatactggaagcgcccgctggcttattttggggaccacattcaggtgggaagtgcgcccgggcacctcggagcgtttctccggatccgcctggtagcagggcgctctcgggtccacgctgcccttgtatggcccgcagcggtgtcgcgtgtttctcttggctcccattccgccgtcccagccgctgtccggctggggaaggggagggctaggcaatacccagctcgctggcctcatgcccagtgccaaccatgtccctggggtattccaggctactgcctcccaggctgactttatttctggggaaagggctaaatcgggctccacagttgcagccggtccagctccaccctgccctgctcttctagctctgggaggagtcaggggtctgaggctctgggttggagaccccaccttccacctgccctccttgtccgagagccaaggtaacaacccaggcatcccagagcccaggcagatggtgtcgagtgacatcacctcctcacagggctggcagcacgctggcaccactgacgtcactcctgcccactgcctggcccttgccctgacccctgggggagactctgacctctccatccttaccagctacctagggtggggtccgcgggtgtgtgcggagtgttcatggaggtgctgctgagggagcatgagaccggaacttccgccagagttagcccgctggggagtgagggcagggattttggagggcagaggatagagcagtggtgtcttcccctggcggtggtgacacaaaagggcctgtgtgccccagcctggcacatgctttgcattcccacactctgagctcacccggagaggagggggcctggaaggaaaggggccttcctcttggccccgagcctaggttgcccctttctgcccctctaccagcctcagctggagctgttcggtgctcagtctctgctcaatctctgcttggctccaaggacctgggatctcctggacggggagagggctggcccaggtggggcgcgggtcggggtgggggtagagcgttcagaggacagggccctctgcagaccctctgagtggcaggaaaaacagctgcagcagcgctgcgaggggaggggcggacagcagccggagctgacacagctgcgggggccccgcctccctcccccaggtgtgcccggagaggctgagcagcctgcgcctgagctggtggaggtggaagtgggcagcacagcccttctgaagtgcggcctctcccagtcccaaggcaacctcagccatgtcgactggttttctgtgagtgcttgggctcctgcaagagcagcccggggagggagactctgcccctcttgtagcccaggacccttctcctcctctgtgtctcctcccattaggtccacaaggagaagcggacgctcatcttccgtgtgcgccagggccagggccagagcgaacctggggagtacgagcagcggctcagcctccaggacagaggggctactctggccctgactcaagtcaccccccaagacgagcgcatcttcttgtgccagggcaagcgccctcggtcccaggagtaccgcatccagctccgcgtctacagtgcgtgaccctgtgtctgccagcagggtagtgagggtggtgggacgtggccagatgtccccctcatttctattttcttgtagaagctccggaggagccaaacatccaggtcaaccccctgggcatccctgtgaacagtaaggagcctgaggaggtgagatgtgtggcaggtgggctgggcagctggtgcgcagccgctgagatggggcttgaagggggctcggaggcagggagactcacaccctgtgctctttaggtcgctacctgtgtagggaggaacgggtaccccattcctcaagtcatctggtacaagaatggccggcctctgaaggaggagaagaaccgtgagtagccctttctcccaaaagccacccttgggccaggtgtggtagctcacacctgtaatctcggtgctttgggaggccaaggcaggaggatctctggaggtgaggagttcaggaccaccttggacaacatagggtgaccctgtctctacagaaaataaaaaaattagccaggcatggtggtgtgtgcctgtggtcccagatacttgggaggctgaggcaggaggatcatatgagcccaggaaatgaaggctgcagtgagctaggattgcaccaccacactccggcctgggtgacagagcgagaccccatctcttaaaaagagctgtccctatggtactacacaggaactgccttatattttcattaagcttttaacttaaaaaaattatacaaatggtacttgaaaatattttcatgagaatttaaactgacaaaaaatctagaagtttcttcttgcctgagaccccccctcccagaaataatctctgctatcagggtgtgttctttcaagcctatttctatgtatttgctcatatatagaaatatttctagaatgatataggcttctgtgttttattatctaaatcagtcattcttaaccaggggtgattttgtaccccctcctcctaggagatacttggcaatgtctggagatatttttggttgtcacacatagagggggtgctactgccatctagtaggtagagagaccaaggatgttgctaacatcctatagggcacaggacagcccccacaataaagaatcaacgtggcctaaaacatcagtagtgctggctgggctcacgcctgtaatcccagcacttttggaggccaaggtgggcggatcacctgaggtcgggagttcaagaccagcctgtccaacacggagaaaccccatctctactgaaaatacaaaagtagccgggcgtggtggcgcatgtctgtaatcccagctactcaggaggctgaggcaggagaatcacttgaagccgggagggaggtggaggttgcggtgagccgagattgtaccactgcactccagcctgggcaacaagagtgaaactctgtctgaaaaaaaaaaaaaaaaaattatcagtagtgctgagaaaccctggtctaagtggtggtgtatggtatacattgttagacaatttcttttatacaatgtttctgggtcagtctatttagatcaactgatcgttttgcttactgccaagttttccatactacgcatagcaggtagtcgagttcaccattccccatttagtggacatctagacggctgctcgtttttatcattgcagcattctttgcacacatccttggatatgagcagacatgaaaatgtttttctagggttgacactgagcagtaaaagtgctgggttgaagggtttccagcttgcatttgtacctggccttctacaggggacagggggctatttagatggtcccctgccaaccccagtggacaaccctagggtggggctggaggtggggctgaggctgagtcttcctccccttcctccctgcccaggggtccacattcagtcgtcccagactgtggagtcgagtggtttgtacaccttgcagagtattctgaaggcacagctggttaaagaagacaaagatgcccagttttactgtgagctcaactaccggctgcccagtgggaaccacatgaaggagtccagggaagtcaccgtccctgttttctgtgagtactaacctgactctctgcttactggggctggggctgctggaaactcctgctggctcccccttatccagcccagggttgactcatgggccaagagctaccccctttgcctatgcagacccgacagaaaaagtgtggctggaagtggagcccgtgggaatgctgaaggaaggggaccgcgtggaaatcaggtgtttggctgatggcaaccctccaccacacttcagcatcagcaagcaggtgtgggggctttgtgctgggtgcaggtggggatgcaagcagagacccaggtggagtggcgggaggtgcactctgctcacctccccaatcctccctagaaccccagcaccagggaggcagaggaagagacaaccaacgacaacggggtcctggtgctggagcctgcccggaaggaacacagtgggcgctatgaatgtcagggcctggacttggacaccatgatatcgctgctgagtgaaccacaggaactactggtgaactgtgaggggctggggacccaggacagggggaccaggctggggcaatagggaactggccccaccctgccctgggtgacatgattgctgtacccccagatgtgtctgacgtccgagtgagtcccgcagcccctgagagacaggaaggcagcagcctcaccctgacctgtgaggcagagagtagccaggacctcgagttccagtggctgagagaagaggtacccaggcgggtctggggcaaggggctgatggggtgggtggggctggcggtgacaagaatgtgctctttcccagaccctctgacccatctctcatcccagacaggccaggtgctggaaagggggcctgtgcttcagttgcatgacctgaaacgggaggcaggaggcggctatcgctgcgtggcgtctgtgcccagcatacccggcctgaaccgcacacagctggtcaacgtggccatttttggtgaggccctccctctgggtagagaccaggtcaccccaagtggggtggtttttaagctctttgacaaaaagccacctgctgccctggggagctctggtgcggagggggaggcaggctagctgggcttgccacctcacctctccctacctccccaaggccccccttggatggcattcaaggagaggaaggtgtgggtgaaagagaatatggtgttgaatctgtcttgtgaagcgtcagggcacccccggcccaccatctcctggaacgtcaacggcacggtgagcgggtgtctcatctgccacactctcctaatccctgcctgcgctttgtggccaggaccagagatcctccccatcccctgagctgcctccttcctttcctcgcaggcaagtgaacaagaccaagatccacagcgagtcctgagcaccctgaatgtcctcgtgaccccggagctgttggagacaggtgttgaatgcacggcctccaacgacctgggcaaaaacaccagcatcctcttcctggagctgggtgagggctgcatccctgcaggggatggcaggggcaggcccaggcacgggcagtaacttgcgcttctcctaacacccctccctctgcctcctagtcaatttaaccaccctcacaccagactccaacacaaccactggcctcagcacttccactgccagtcctcataccagagccaacagcacctccacaggtaagccaggcctggcaagagaacagggctgtgccagggcatcctttctgccctgtccctccccagagagccctgtccagaaaggtgagtagcagccccatcttgtcggccctggactggctgggccaacgatggtgacgaagtggcctggggcagggagtgacgaggagtgtctttgtggcgcagagagaaagctgccggagccggagagccggggcgtggtcatcgtggctgtgattgtgtgcatcctggtcctggcggtgctgggcgctgtcctctatttcctctataagaagggcaagctgccgtgcaggcgctcagggaagcaggagatgtaagcccaacaccggcccctccgcccccgcccgttttgccacccagcctgctgcctgcccagcgcccgcctgccccttgacagcaggagtggggccctgggtcagggtgctgcctgctccactacccactgcccagcttccatcagccacatggtctcatgacctgcttccctgccctgggctggccctggagcaaggagccctcatccattccacatccttcacagcgggcccttctcttcctgtggccctgagataccaacctctcacctccctccctcctcccagcacgctacccccgtctcgtaagagcgaacttgtagttgaagttaagtcagataagctcccagaagagatgggcctcctgcagggcagcagcggtgacaagagggctccgggagaccaggtaggaggcggttcctgtggccagagcctgccacatcctcagggaccgggggcagtagggactaacggttctctctatccttcatccctctttgtcagagatagggagcaggccccatcttgccttcttaacactactcacagatggcagggccaagcaagtggctgagggagcttggaggaagactgagaaagaccctgtcccatcaccttttcctgaccaatgcccgtggctctgggtgtggccatttcaccagtgcagtcagacgggggccgccctccttccctggcctgccatgcccatcctagcttcctccccaacgctgcccaccaaattcatcggcttctgcttccatctctccttcctctcggtgctcacaccttcaactcagcaccgtctccgtgttcccctcttctcctctacccacttttagggagagaaatacatcgatctgaggcattagccccgaatcacttcagctcccttccctgcctggaccattcccagctccctgctcactcttctctcagccaaagcctccaaagggactagagagaagcctcctgctcccctcgcctgcacaccccctttcagagggccactgggttaggacctgaggacctcacttggccctgcaaggcccgcttttcagggaccagtccaccaccatctcctccacgttgagtgaagctcatcccaagcaaggagccccagtctcccgagcgggtaggagagtttcttgcagaacgtgttttttctttacacacattatggctgtaaatacctggctcctgccagcagctgagctgggtagcctctctgagctggtttcctgccccaaaggctggcttccaccatccaggtgcaccactgaagtgaggacacaccggagccaggcgcctgctcatgttgaagtgcgctgttcacacccgctccggagagcaccccagcagcatccagaagcagctgcagtgttgctgccaccaccctcctgtctgcctcttcaaagtctcctgtgacattttttctttggtcagaagccaggaactggtgtcattccttaaaagatacgtgccggggccaggtgtggtggctcacgcctgtaatcccagcactttgggaggccgaggcgggcggatcacaaagtcaggacgagaccatcctggctaacacggtgaaaccctgtctctactaaaaatacaaaaaaaaattagctaggcgtagtggttggcacctatagtcccagctactcggaaggctgaagcaggagaatggtatgaatccaggaggtggagcttgcagtgagccgagaccgtgccactgcactccagcctgggcaacacagcgagactccgtctcgaggaaaaaaaaagaaaagatacgtgcctgcggtgaggaagctgggcgctgttttcgagttcaggtgaattagcctcaatcccccgtgttcacttggctcccatagccctcttgatggatcacgtaaaactgaaaggcagcggggagcagacaaagatgaggtctacactgtccttcatggggattaaagctatggttatattagcaccaaacttctacaaaccaagctcagggccccaaccctagaagggcccaaatgagagaatggtacttagggatggaaaacgggcctggctagagcttcgggtgtgtgtgtctgtctgtgtgtatgcatacatatgtgtgtatatatggttttgtcaggtgtgtaaatttgcaaattgtttcctttatatatgtatgtatatatatatatgaaaatatatatatatatgaaaaataaagcttaattgtcccagaaatcatacattgctttttattcatcatgggtaccacaggaacctgggggcctgtgaaactacaaccaaaaggcacacaaaaccgtttccagttggcagcagagatcaggggttacctctgcttctgagcaaatggctcaagttctaccagagcagacagtcaccctacttttcagcagcaaaactgccctgatgacgcagcacgaagggcctggcaggctgttagcaggagctatgtccccttcctatgctttcctgccacttggaactgccctccagacagcagctcaggctcaccgccatgaggaagggggccttggcatttggtcctacggcaactcgagtattgcctcccaaccccctgaatgagcaagaactcaagtctgctacaacaaaagaaactttatttaaaaatatttttttacagacatgggtgcttgaaaaagctctttagttaactaactgtgtatggaaatgaaaaaaaattaataaataacattagtaaaacagacttctaaacattaatacactggcaaaataaaatggacttaaaatgaaaatgaatcttaaagctctgctattctctgtaagaatatttacctt

SEQ ID NO: 8 - DNA, 36199 bp, Homo sapiens
  source 1..36199: mol_type = other DNA
cacatgtgcagatacaccggctgacagtgacagcacacttatgtgtgtgaaatgctttatgtgctcaatttcttttaaatctcacaggaaacacacgctatgaggaactacccacatgcccacatcaggcaggaggaccccggcgggtgcaagggtggaagtcgtctcttcatggctgcacccttggccagtgccagtccagcagcctctttcaaagaagcttttataggcagatagtttgtcagcccagctcgaaatgtgaacaaggtagcagccgagcccccacccatccggctgcactcccctgcatgtccagccccgagccctgtgtcaagcacacctccttgggaggagaccgccccagcctgcaaggaagtgctcattatggagagtggagagtgacaggccactcccggccacgtctccaagcaagccggtgccccctcctcctgctgccaccgcccactgagctgcctgggctgcagacgccctggttggagcgtctggggccagatgttatagggcaagctaaaaataaccgggcaggccagccaccatcgaggaaggggcatgtgctggacgcggacacatgatccgagggaccctgctgggtggaactaagaaagtccagcagactgtgcacgctcctgtccccactcacaggcccacgcagcgaggggggcccctcttctgtgtgcgtctggaaggtcgctgcccagggaggaaatgcctttaaccggcgtggggccgggcagctgcaggaggcagatcatccgggctctgtgcctcttgctacttctcctccacgccggctctgccaagaatatctggaaacgggcattgcctgcgaggctggccgagaaatcccgtgtaagtgcctgggatggggaggctggggcagcctgtgtgggtgggagtcagccttgaggagaggcgatccctagacactgccttctcctgtaagtgctcccccaaaacgtggcaaggcaggcgtcttcctatccgtgtgtgtctgagttgtgtggtgggcagatggggctcacccccaaaaaaggacccttatctgtgaatcaagaggcaaaagacacatcacaaatgtgccaaagaatcgtgccttacttttgccttgttacaaaagtatgcttgctcctgaaagacagcatctcatttcggaaccccaggatttaaacggagcattccgtctccctgatcagtaccaaatgtttgtgaaatacacttatgcattcatgtggttgtttattcattcagcaaacagaatgaatacactctgttaaacagaatttagcagaaaccgctctggggaaggcactctgtggggtcctgtggatacaagataaaggaggtaccagcccccatcctcaagggtggcgtggccgaggtgcagaggcagttgggtgacaaacgatggcagtacaaagagaaggcatgaggcagtgagtggcaagaaacacatgccctggggccagatgtctgggtgtgaatcgggctctgctgctcattagctgcgtggcttctctcaggcaagctgcttgacgtctctgaaccccagcaatctcatctgcaaaagacagagggatgcatttcttcacacaactatgtctgggtcgccactgtttaccaggtactgtcctaggctgtggggatacaggcctcacatctctgcctggggaactcaatggggggctgggggcagcaaagaataaatgagagagatctcatttattagtatgtgcagattgcaagtgctgaggaagcaataaagcaggaagggaactaggagagagaatggggtggaggaggctatcatttttggttcctcccaccccccagagatcacagatccgcagggacggagtgagccaggacgatatcagcacacagagggctcaagaaggtaatgctggcacccacagcacctgtgtgaggattgaatgtctcagtatgtagaacgtctttgcaacatagcaagctggcagtgcatcttggcagctttcaatcaaataattgtgactttgtttggtgttgcctaacagggtagcatgtaggttccttggggtagggcagggttctgtttcactaatgtgtgcccctggtgcctgccattaagttgacgcctggtacattcttgtgggtgaattatgagcggctgcagtggtagcacgggatgcgtgtgcagctcttccccaggagagtcagacagggtttttagggttggggacagtggacctgtggctttaaaggataaataggagtttgccagctggagaagagggaagggagaatgattaatgaccccaggcatagtggggtgcctgaaactgtattgagacaaatctgggcctccagggctcatattctccatgttcggcacatgctcgtggcgggagacaggaggacttcgagaagtggaaagagtagtagagtgacatgatgaggcgcttggagaagggaaagctcatgtgagatggtgtagtcacctaaggcttcctggaggaggtggagaccttgactggatcttggggaaggattaggaattaggaggaggaaggaaattcctggaagaaggctcagcatgagccgaggctcacaggcagaagtggtgcatctaagagctgcttgggagaccagacctggccagaggggaggcattgaggaggggtgagagactcttctcagattttattgactcctccatgctcttctctggtgcaacaggatgatttaaccctcaactgtagttagtagcactttggttctacaaccatagagagattgccaatatgtcatctgattttatctccaccatgtcccaagatgtcataggagccagcactgtgacatccacataggagggtcagggttgatatgcgcgggggatttgggggagtctctaaaacacaggcctgggtgggctgcaggagctgattcacttgtagccaccactggtcaggatgcaagtgctttgaattggctcctcgctgaattgaaactttccctggttatcaacagatattggcctccttcctcagcgcctagacataggtgaggtttatgcagaacaactgtgccagccaggcacggtggctcatgcctgtaatcccagcactttgggaggccgaggcgggtggatcacctgaggtcaggagttcaagaccagcctgaccaacatggtgaaaccccatctctactaaaaatacaaaattagctgggtgtggcggcaaatgcctgtaatcccagctactcagaaggctgaggtggaagaattgcttgaacccgggaggtggaggttatagtgagccgagattgtgccattgcactccagcctgggcaacaagagtgaaactctgtctcaaaaaaaaaaaaaaaaaaaaaaaaaaaaaactgtgcctcagagaaagccctgggcgaagacaggctgttcgcctgtggctcagaggacagggagggctgggaagagcacctggcctggaacttatcacagtgaaatctctaatgtgctgtgcaatcttggaccactcgctgcccttctctggtcctcagtcctcacctgggaaatgaagatggagaacctatgactttcatggccctttcgttcgacagggacttacggatgataggaagcagcccccacatgcacaaagtcgagacttccccgtgaggcaccactccctcaccctacgctgagtgagaccttcagccttggccctgaccctcaagggctatgggctcgtcagagtgaggagatgctggctgtgtgctgaactggcttgcaccccctctggagagccagttttgttccacacagccatgatcacaaactaagttatataaacttacagttaaatcatttgtattaaaaagaaaggaaagaattttttttttttaagacagagtcccgtggtgtgatcttggttcactacaacctccacctccccggctcaagtgattcttgtgcctaggcctcctgagtagttgggattacaggcatgtaccaccacgcctggctaattttttgtatttttagtagagatggggttttgctggtctcaaactcctgagctcaggtgatccgcccgcctcggcctcccaaagtgctgggattataggtgtgagccaccgcacccagccaggaaatacttcttgttagaaatgcatcactttccaattgtattgctccgttttgctattctcttggctcctggggttttatgtcttttgcatccgtgtggtatgaccaggagagaagctactccacatctcttcccactcccattcagtggcctcacgtgggttgcctgaaatcaaccatgctgggagtatttacaccttggaaattggccagggctacatgaggagagctggtggttcagtcctgaccaagcgcagggctgcctgtagcctgctctagtctctcaaagcctcgtcccccggcagagtgagttttgtgtagcggactggtctcttagagtggggccggttaatgtcctgagtgctgtttatgcttcttggtctaagcatgcagagacagtgccagagatgagcacgcttgccttccattcccgtgggaggcccttaggagaagcttctaatgagaagtgccgggtctcccgccttctatctctaagaccttcaggagtttccttctcctatctgagcctcggtttcctcatctgcagcacgggacacagtagtgcctgctttgcagggctcagtaaaaagaccatagagcccacagttccctgactggcacagaactgcattccctacatgtgcactacggacagatttaaaaacccattccaggccgggcacagtggctcacagtggctcatgcctgtaatcccagcactttgggaggccaaggcaggcagatctcctgaggtcgggagttcaaaaccagcctgaccaacatggagaaaccccgtctgtactaaaaatacaaaaaaaattagccgggcatggtggcgcatgcctgtaatcccagctactcgggaggctgaggtaggagaattgcttgaaccctgggaggcggaggttgcggtgagccgagatcgtgccattggattccagcctgggcaagaagagcgcaactccatctcaaaaaacaaaaaacaaaaagcaaaaaaaaaaaaccaaaaaccccattccagaaccaaatatttatgagaatgaataaaacagaagcagaacgtttgtaaatagtccaataacctttccatggagctcagtcggttctgtggacttggtgtttttggtttttctttcctggcattagatgaaactcctgagaacaatgatttactgagcgcttacactgtgtcagggcaagagtttgccacgtattcgctcttgggagcctctcgacaaatccaccgggtggggcccatggcagccaccatgcgggcctcggtgctatgcagcccctggcctgggcaggctggcccctactgtcagtgtgactgtgggtctgttttcccctctaagcacccctccagggcagctggaaatggtggttcagtttgcaccctgcccagagatgcctgtcctgctgaggggggctgggaggggctagagcagccaggagctggtgccactttctctttcaaatgcatcctgccagacggggggagggaggggagcattgcattgtctatctggaggcaccccctgtgctaatagcagtaggggactgtaggagccagcagtggcccacaacttgcattcctcagttccccatctcctttcccccattatttctgtgagctcttgagagccttccaaacatccttttattttttgctgagatagcaagagtgggagaagcagatggcctggagctgtgctttcaccattccatgagttctgaccttcaccttcatctttgtaccgtaagcatctgtgtctgctggcctgaaggctttctctggctgcaggagcctgctcagcccacacacagtcaggccaaggtgccggggaggtaacacctgtaggagcaaatggagctggagcccgatggcccagctggcttctcaactttggtgggacacgagtgaggagtgttccacacggtttctccaaggtccactgggtttgagctctagtgcccaggcagccacctgctcattaacactccctgaagggcgccttccctttcctgcccagcttcccactccccagctggggcttcctgggatcccctcccaaatgtactccctgcactcgaatcctgtcggatccatgctgtgggttgaactgtgtccccccaaagatatatccaacccctaacttctgatccttgtgaatgtgaccttttttggaaacaggttcattacaggtgtaattagtcaaaataagatgaagtcatgctggaggtgggtggttccttaatatgaaccccgttccttataagaaaaggagaaggggccaggcatggtggcccacgcctgtaattccagcactttgggaggccgaggtgggcggatcacccgaggtcaggagttcgagaccagcctggccaacatgctgaaaccctgtctctactaaaaatacaaacattagttgggcatggtggtgcatgcctgtaatcacagctactcaggaggctgaggcagtagaatcacttgaacctgggaggcgaggtggacgttgcagtgagccaagatcatgccattgcactccagcctgggcgacagagtgagacttcatctcaaaaaaaaaaaggaaaaggagaagagacatagaatcagagacacatggagagaaggccatgcaaagacagagggttgcagaggtcagagtgatgtatctacaagagacacatgagggaccagagaacatgagggactgccagccaccactggaagctgggagagacattgaaaggttttctccttagagcctccacaaaggaaccaaccctgccagacacctggatctcagccttctggcttccagaactgtgaaagaatccacttgtgttgttttaagcccccagcgtgtggcattttgttgcagcaccctcggacacgaatacagtctgctctgggcatcaagggagtcagcttctgttggttgcaaccggcaacgagacatttcacagataaggaaactgaggccattgcagagaatgacttatctgtgctgttttggctggggacaagtggggctatcactatatccccagagtctgggagctttgtctgctccttgaaccaggtgagctcacaccctgcgggacttcatcatcaagactatgaagatgcctacgtcttgggggtcagcgggtgctcagtaggctccggaaacccccagcagcacccaggagggggtcttcaaggctcatggttgagaaccccagaaaagccccggagctgcctgggtgctggagagaggggatatgtcgtactgatgtgaagcaaacttactgtaggcttggccacaccctggtgtctagcccagaacctggcatggaggggggactggaaggaatgaaaagtccatgaatagctgacttttgagggggaccttaggcaaatcactttctcctcgctgggactgagattcctcatctgaggtcccagccctgaaattccaggtggaaatcaacgagtaaagaagcatcccaggtccctgtgtggttatcactcgggagagggagacaggaaagcaggacacctctgtgccagcactcaccgcagccacagggcagaggcagatggatgcccatggggtgagcacatcgcacctgtccactcaggggactagaactcagccatagaaaggtacggagcctggacacacgccattgcacagatgaacctcgaagacatgatgctgagtgcacaaaggccacgggtgtgtgacactgtttggatgcaacgtccagaagaggcaaatccagcgggacgggaaggagattcttggttgccaggggctgggggagggaggaatagggggcagctgtctgatgagaacagaggttctgcctggggtgatggaaaagccgcagaaatagatcatggaggtggttgcacacatcgtaagtctaattaatgccgccaactcgcacacttaagatggctgaaatggaaaattgcatgttatatatattttaccacaataatttttctaaaaagcagcgagaacctgaggcacctctcgggaattctgcgatggcacatagagtccttgtgagtcctctgtgtccccagcaccgagaggcagcctctcagcaagtgtccgctcacggtgcccactccggctcgtgggtcatcgtagcttcacgcccatctcctccgaggcctctccctgaatgccccattatgatttcccttaaatgtttacataagagcccagctcttccttcttagcatttagctctgttgttgttattggcatgatttgtttaggtttctctctaactgcttttgcagatgaccacaaaaagcaattcgttcgccttatctgtgtgctgttgtgtgtggtgtctctgacttgtcctgaaacagccacgtgaagtcagggccaacgtcttctggttcaactcggaactccccatttccagcccagcgcccgaaacccagtgggcctgggaattatgaatttactgttgaatgaatgaatcaattatgttaaaaccttccggaacagaatagaagggatggaaagttctagaggactctcgggtgcactgactgacaatgacattgttgggaagacttggtcgactttgccgtctcgtgaatttctttctttcttttttttttttttttggcctccctctgaggaagtcccattaaccttgtgccaaattctgtccccagagcccagtgttaagggccccgggcaccaggaaatggcattcatctacgcacgctgaactctaagcccactgttccggggagcgacatatttttagatgtttgacgtccaaattccagaggaagagcaagtaatgcttagaggaaaactggctttcttcctccaccacgcaaaccagtgctggggtgcatttttattggtctggccgctaagctgctggaactttccaaagaagggtaaaagctcctgcatgtggaaggaagggggtggtgagcactgagggaggcatgtgggtagctttggtggcagctctggagattggattctttttccctaaatggaagtggtttataaattcttctactgattattaaaagaatgatgtttactgtaaaaaaaaaaaaaagtcaggcaacacagaaagttataaattagtagaaatgatctgaattcttcctccaagatgtaaactctggtgacattctggggtgtctgtctagattgtttcatgaagcagttacacacagcacacgtgttttctcttagtggtattttattcgtactgattcagatttcttttattgaaatgaaattcacatgacagacaattcaccctttcaaaggtacaattcagtggtgtttagtatactcagagtcatgcaaccgtcctaactgcctagttttggaacgttcccatcaaccctacaggaaaccctgcagccattagccgtcagtcctccttccctcttcccgcagcctctggcaactaccaatccactttctgtctttatggatctgcctgttgtagacgtttcatggaaatggaatcatccagtatgtgctatttggtgtctggcttattcacactgttttatatgttgcattttcacttaataatctgtgggaagtatgttaacatggtggatcgacatcttcctttctaaggcctgcctggtgttctctgtgccttcacttatgtatccagcctcttttggttgcacatccagcttgtttgctttaattgaatctggacttggacttgctgaattggggttcccactgctgcagcccaaaatgggcatttccccacctgcttcctagggatatcagtggcagaatggctttgcaaatctatttttgttcatctatatgaacaaaaggtcatatagtttcactcttgtcacccaggctggattgcagtggtgccgtctggctcactgcaacttctgcctgcgtggttcaaatgattctcctgcctcagcctcctgaatagctgggattataggcacccgccaccacgcctggctagtttttgtatttttagtagagacgggtttcaccatattggccaggctggtctcgaactcctgacctcaggtgatccacctgccttggcctcccaaagtgctgggattataggtgtgagccaccgcgcccggccaggaatcatttttatccaggtaaggcatacagaaccggagggttcttgtctaagttgtaccatgaatgtgtgctcagtggctactttgaaatagacttactgtacaatccctgagcaggaatgaaaataaaagctggaaatcacgggttgctctggggttttcaggaaggtgtcgtccgtctggggtgagtgtcctcctttctcagggatttcatgtggcattattttgttttcatctgagaggcaacataagctgggagggaagggctgcaaagagggggccaggacaaatgtgtacctgggggcccccattttgactccaccctgagctggggctgggccacaggagggcagagggaacactttgggagggggagagagcccctggactgaaagaatgggaagaaattgacaggcaccattgggaagcaactcccagttccaggctcctgggtcaggaacgcccatggagtaggacagacccaggttcgagtcctgcctctgactcttctcagctgcttgctcttgggcaaatgcttttagtttttttgtgcctcagtttcctcatcagtaaagtgggtttagaatagcagcagccccagagggtgatactgaagattcagtatgtgaggacccagcacagcattggcacacagtaagagcttactaaatgctgcctcccttgccataagcatgaatcacacagcccaggctctaagcctggtgtctgtgtatggtgactgtgtgcctccgtttcctcacctatgcaatggggacagcagtggtacctactggggagggctggaggcgacttcaggtggtcctccgagggcagtgctcttgcacatgctgggtggctgtttccgcatggccccttctcttaggaactctgcgagttgtttgccctggtcttatccagaaatgatgaggttatatccaatcccacttttcactgttgctggtgggtttctcgtacgtgtatctgcagcctcagctggactggagctccttaggtttcttctgcatctgctgagcatctaacctaacgctatggagatggtgggcgttcactaaatcatctgggagtgaattgagggaggatacagcggaaatgaagaaagactggaagatatgaagaatgaagttactatgggacgagaggcttctgagtccattgattaaaaataaaaggagccaggcgtggtggctgacgcttgtaatcccagcactttgggaggctgaagcaggcagatcacctgagctcaggagttcaagaccatcctgggcaacaaggcaaaaccccatctctactaaaaatacaaaatattagccaggcgtggtggcgggcacctgtagtcccagctacttgggaggctgaagcatgagaactggttgagtctgggaggcggaggttgcagtgagctgagatcgtgcccttgcactccagcctgggtgacagagtgagaccttttctcaaaataataataataataataataataataataataataataataaaaggaataaaaatcaggtgaactatggattagcattgcccgtgctgagccgagcacacgcaggtgtggtctcttttgatcttcaccacagacatcggcagcaggtattacttttgtgtccattttgcagatgacgctcagatacgttagtgattttcctgtatgatcgcacagccatggggttcaaacccagacgcgtcagactcctacacctgagcccttaatccctgtgcaaagcagcctttgatgtataagagatcatgagcttcggtcccttccatcttgggatatggctcctaagttcttgccccaaaggatgtggaggttcagctgtgaaaaagagttaggaagcgttccgagtagaaggaacagtgtgcggaagcctccaaggccaggaggagcagcgaatcctcagaaactgaggatggccgagtccagctttacattttgaaagcatccttcctcatctctggagtgtgggatatcaagacccactcccctaattcacatccgcattaagtgagcataggagtgtgatgatgcttgcccatagcacagccttacacaaacaaaagggctgcccccacacctgtgcaggacccctgctctgcagaacccactgcacagggcccatgtgcagatcagcgagttaatgcatgttaagcacttagaagggtgcccagcaaactgccagggtgtgttgaccaccatcatcctcatcttcatcatcatcatcatcatcacagcttttgttccacccgaagaatatttactgagtacttgctatgtgccaggcactgttctacgagccagggacacaggtaagaacaaaaccaagttcctgtccctaggagggagaaaaacagcaaacaaatgagttcataatacacagtccacccaagggtgatgaggtgtgtgaagctccacaaagcagggagagggggaagggaaggctgtatgagcagatcccaggtaggtgagagccgggcagatatggtgggtgggcggggaaagagccagtgcaaaggcccagaggtgggactgggtggctggagagaatgagtaaggaggggagtgctggaagagaggtcagagacgtggagggggtcaggggagtcaattccagtccgacctctaggccaagaaccttggctgttaactcctttaacccatggggccagggataaattggagggtaagctgttcatgtctctgagtaaaagctatttcccagaactccagaaaacgccctgtgtttagaaccctctttgagtgtcaccttgcgtgacatgaaatatttttcaaaacatgtaatcctttaatctgggcctctcagtgaaatcagtgctttatcactcacgaccatctgttgtttcagtcacgcagtaaataactcgggagatgtggcttctaaggcgcttatctctttgttctctccaaattctgagaaatgacatttgaattttcctctgagacggcggcacagctcaaattgatctctgcgtaccctcccatctgggactgccattaaggaagtcccaggactgggaggcatttgtggtgggtgaaaagtgttttgcttctgatcaccaggttccattaaatttcataaatgaggcttcatcgaaaaataagctgggcggaaaaataagccgccccctcccgacgacgtacggtgtattcttaaccaccctccgaaggaggctggggcgcctgtcatgttagattaggattaaaaattgcggaccccaacatgggtccaatgtagttaattctgcagcaagtgaactgagagctaatggcgtgggattatgcacctgctgcggctggaaagttattggtggtggttacaggggtgagggagattgggcggagaaaggggtgataaaaggtgataaaaggtccacgctctggtctcccctctgtctttggggaaaaccattcacctccttaaggcctcgtctccatatccaccaaatgccagcttagtggtcattttcacttttcgtgaatttaacagccaatgtttacaagagtaaaaagaagggttctctgggtgaagcagggatgggggccaggggcccccctcccaggctttcctgccgcaccaccccccgctcctgcttggccattcctgctctctgcccctcatgccctcagtctcaggccctgaggctccctgggggcagctgaaaacctctggccttggtgattccaccaattcccaggggcacccggcccggacctgttcccagggacctggtgccccctgcagggcactggtgacaccgtgtgcaatttgcagctacccagataggcacgcaggaaggactgcgctcggtcccagccaggcagggacagacacagcctctgctctcagtgtggggtggcgagggcctaaaaatgaagaatcatactagaaacatcccgacgagtctggggaaagctgccctcagaaggctgtttaagctgaggactaaagggtgatttggccaattgaagctgatgggaggccttccaggcagaggaccagcacgtgccaatgatgatcggtggcactgtgctagatgccagcctgagtgtagatgcccgcctgggtgtacactccatcctgagtgtagacaccagcctgggtgtagacgccatccttggtgtagatgccatcctgagtgtagatgccagcctgggtgtagacgccatcctgagtgtagatgccagcctgggtgtagacgccatgctgagtgtagacgccagcctgggtgtagatgccatcctcagtgtagatgccagcctgggtgtagatgccagcctgggtgtagacgccagcctgggcatagatgccatcctgggtgtagacaccagcctgggcatagatgccatcctgggtgtagacaccatcctgggtgcagactcaggataccccaggagggaagttctgtagggagaaactgatgcttgcccaaggtcacacagcaagtgagtggcagagaggatttaagccctagagccgggatttgaaccaaggccgttagctgtggagcccgctctggttgcccgtctgctctgccggatgagacaagctcagctggaggaaacggtttgaagagatgaatgtgcttggagggcgaggggaggagggagggcagtttgctggcatgtaaggacaaggagggtcggggcgatgagatgaggccaatgtatcctatatctaagttattaaatcgaaaaagtaacatgtacctggttggcgcagggctcggaggtcgagtggggagctgggactttgtcccgagggtgctgggaagcctctggggccttggagcacagaggtgaccaaatccacaggtgcctttcataaagcctctttgggctgcagtttggagaatggatggatttggaggcatcgaggccagggacgaggccactgtggaggcactgatggggacttgggcctggggtggggttcatttatagaatgtgcctggcagagggacagatatcaagtttccaggggccgccgtaacaaatgaccacaaactggggtgggaggggtggttaagccaacagaaatgaattgtctcatggttctggggccagaagttcagcatcagcttcactggactgagatcaaggggtcagcagagccgcgcccccccaactccagaggttccttccttgccaatccctgttccggtggctgctggcaatcatggtcactccttggtctgtagacgccatctctgcctccctcttcacatgactttcttgtgtgcatgtgtgtgtgcgtgcgtgtgtacatgtgtgtgtggggggggcgtgcttgtgtgtgtgtgtgtgtgtgatctccctctgctcccctcttctcagacacttgtgactggatttagggcccactggaaaatgcagaatcatctccccatgtcaagatccttaacctaattgcgtctgcaaagaccctttttcctttgcatttaggattccagggagtagggcctggtgtctttggggacctttattcagccaactgtatgtgcaggaggagacacaggaggctcgaggccctcccttgaaggggtcgctcactggttaaatgcgatggcggatttctattaagacaccgagaaagagtgacaagggagggtgggtgtgggacatagaagcgggggtggtgtggccctaaccctggcctgggagggctggtgtcaccgaagcacatcccagccttgagcgctgatttgagtctcggggaatggtccctctggaagacctgaaggaacgggcattcggcagaggatcagcacgggcaaaggcctggaggtgacggcgcccacactgtcatcaaagcagcttcagtagctttgtgaggttctgtgagtcaagtctagattaagccaggacttggatgatgtgccctcgctctgcatggagactgcctttaaattgaagaagaatgtgtacatggtaaaaagacattgaaccactgcagaagggtgtacaatagaaagaaatctctctcagctttacaccctattcttctctccaaaggcacctttcctaatggtttcttgtgtgatcttgtgtatccttccaaaaacgttctcagcctatgcaaatagatatatatatgttacatctgtgtgcatttttttaaaaacacagatggggcagagcctacacgctgttccgaaccgtggttttcttcatgagccgccatatcctggcgatctttcatatgtggcacattgagtcttatttcattttttgccaacaccttaaaggcaggttttcatggctgtctactattccaatttgcagatatgccctatgtactgggccagcacaagggggccagattttgaatatgttgggggaggacttgtggggaggtaagtaggtgaagcattttagattaaaggaacagtttaggcagaggccaggaggcagcgcaagccagggtggtttttttccaattaatctatcctgtatctaactctgtgtgtgtgtgcaaatgagcacaaactgctcaccacgcaccccattttcatagaagcagaagagtgttactgggcctggatggagtctggtgtcaggggacccagggcagtttgcctggtggcttaaactttctcggtcttacctttaaaatattgatcttattggtcactcacccatattcctatatgttaagtacagtactatatcttttttttttttggagatggagtctcactctgtcacccagcctggagtgcaatggctcgatcttggctcactgcaagctccgactcccaggtccacaccattctcctacaggtgctcgccaccacgcccggctaattttttttcttttttttgtatttttagtagagacggggtttcaccgtgttagccaggatggtctcgatctcctgacctcatgatccacctaccttggcctcccaaagtgctgggattacaggtgtgagccactgcgcctggactttttttttttttttttttttaacagggtctcactctattgcccaggctggaggggagtagtgcgatcacagctcactgcagccttgacctcctgggatcaaaccatcctcccgcctcagcctcccaagtacctgggactacaggagagaatgagcatgcctgactgctttttttttttttttttttttagtggagaaggggtttcgctatgttggccagcctggtctcaaaacttctgagctcaagtgattcacccaccttgacttctggaaatgctgggattatacgcttgtgtgccacagcacctggcccctatatcttatttttgaactttttattaaaacataatacttcgggccaggcgcggtggctcacacctgtaatcccagcactttgggaggctgaggcaggcggatcacctgaggtagggaggtccagaccagcctggccaacatgtagaaaccctgtctctactaaaaatacaaaattagccgggtgtggtggcacatgcctctaatcccagctactcgggaggctgaggcaggagaatcgcttgaacccaggaggtggaggttgtggtgagctgagatcgcgccactgcattccagcctgggcaacaagagcgaaactccgtctcaaaaacaaaacaaaacaaacaaacaaaaaatacttcaaagcgtataaatcctacgtgttcagctccatgaattcccacaaaataaacacaccatgtaaccagcccccagaccaagaaacagaacagagccagtcccaagaccacgtgttcccagccagtcactgcccaccagattcctgacttctcatgccagacatcagcttggcctgtttttgagctgcaaatataagtggaatcccactgtggtgttctcttttgatctggcttttttcctttctttattttctgagatgtagcgatgttgttgcatggatttaatgattctgtgtataatttgtaaattctcaaagctctatataaacatggattctaagaaatccacacatatgtacatatacatatgtacatatagacatatgtgtgtacgtatatgtatacatgcagacatatatgtatatacacatatataccacatatattatgtattatgtatcatctctctctcacaccgggtagatacttaagtaattgtccttcagcaaacacaccaagatttgcttatccttttgctctgggctttcagttatttcgtgccgactgttataaaaagatacatggtatcttctgaccttctgagccgccctccgaggctggctctgcctgcttgctgtttctcacaagaggaaacgcaggctcagagaggcccggcgcactgcccacccccttgcaagctccgggtggcgccgggactcgaacgcgcgccccagagctgctgacaccgccctctccccgcaggccgaggaggcgggcgcgcccggcggcccccggcagccccgagcagaccgctgcccgccgcctccgcggacgctgccccccggcgcctgccaggccgcgcgctgtcaggcggactccgagtgcccgcggcaccggcgctgctgctacaacggatgcgcctacgcctgcctagaagctgtgccgcccccgccaggtaggtcctgggcccgagggagggggctgagggaggaggacaggtgaacagagctgtcccgggggaggggaagaaaggagctgggcttaaagctgggccacctgggcgggtctcgggcgcctccactgcgcacctgtgaactgggacgggcgctccttgagcaggcgtggcgctgctgtgcagctgctgtgtgtcccactgcccctggggacttgcgttgtaggagatgcccttccagagaaaatgccacttcctatgaaaatgggatgggggcagggaccctagatgcctccagggagccacagtccagcagacacccattcccgggaaaatgcccgctgataggatgcgggtgcagtgggatttctcatttaaaagatcaagattcgaggcttttctttaaaataccggaccatgttccctgggctgccaccagctggaacctcaggtgcccctggatgaggaggtgtcctctagcttcccactcaccccatcaacccaccaggcccgccacacttctcatgcctggccctggggtatttgaggtggagaccaggagcttagcaatccagatggggcgtgccccagagaggagcgggtgcgtggcttcagatccaccggccagctgtcactttcttgagtctcctctcctgtgaaatgggtggatcacctgaggtcaggagttcaagaccagtctgggcaacatgctgaaaccccgtctctactaaaaatacaaaaattagccaggcgtggtggcaggagcctcaaatcccagctacttgggagactgaggcaggagaatcacttgaacccaggaggtggagcttgcagtgagccaaggttgtgccattgcactccagcgtgggtgacaaagtgagactctgtctcaaaaaaaaaaagaaagaaaaagaaaagaaaagacaacgtgcagctcacggtggacctgccactccaaagtgctcagcactgcgcggggctgggagcagataatgagatgtgtgttgaatcacgatgccaacctctcggggagaaacaatgttcagggattcatcattggcaggacggagtttcagatcccttttgaagagaaggtttgggaagggaaggaggtgaccccgtcggggacagtaagtggggagtagggtagacaagtagctatttcctggggaaaatgtgcccttgggtggggaaaatgtgcccctaagcgggtaggatttgcggcaaggaagtcttctccaccctcggctctgctcagggtgataacaacagctgtttggactgtaccttccacgagaggctgtgggttcccatgggccttaattagggaaggcggctcgagaaggtggcccagcccctcctcccaaggctgagaggagagtcagagatggcaaggatgacctcactttgttttctttggggtgaggtcacagcgatgacaggggactcagcacacttaatcatgctcataattacagtcttataatgctcatcaagacagacagggactatttcagtactgcctctgggcaggaactggccgctgctccaggccaggcagcctcctactcagccttcataagaaaacctaaatgttgcccctcatggtggacaagcacataaaggcactcccctctgggtggacttatcaaaaatggtgccccttcctctggaatgtgacctgacatagggattctagccttgcctcctgcctatagcaggtgcttttgagcagtgcacagatgatacaactgtacatggcattcccaatagccttttcatacatcatctcgtttcacgctcacacagcttgatgagatgactgttatcagccctgtgtcacaactgaagctcagagaagctgaatcatttgcccaaggtcacacagcaggcagacctgcagtttccatccagttgtctgactgtagagaagtgttcattgccttaggccgtgagtgtgggaatgtggagcccctgccctggcacctgtacccacgtgtggggtccttcattgccaggaatatcctcccccagacatcccatggctcacttccttgcctcctttagacctatttccaaaggccacttttcaggaaagatttccctctctcaatgacagcccctccttggccatttttaaaccctccatccctgtttaatttttctctgtagcgtaatctggaagactgtgttttacttgtttgttcattgactgttctctccccacttgaaggtcagccccacagggcagggagtttgtctttcattcactgctgtaaccccagcatctaaaacggtctctgggcatataatactcaataaatattcactcattcattcattcactagccagcaggaagagcaatcatactactagtgagtaataacaacaatgctgtctaacaccagatttcacaatcttggcatattgacatttgggattggttgattctacttgctgggcaggggggcgctctcccgtgcactgtaggatggtcagcggcttccttggcccttccccaccagatgccagtagcaacctctaccccaagttgtgacaaccaaaaatgtctctaatctttcctaaatgctctctggggaacaaaatcgctctgcattgagaaacactggtctaactcaggccctgttccaggcaccttacttcttcattgccatctggatttgggtctgtccaaagcagacccagagacaaggacttgggcccaggttgtttatttgggaggtgatcccaggtagttccagtgaggtcatgaagagctgagaccccagggccaccaggcctttaagggtcttcaactagagtaatggggagcccggaccaagtttcactactccaaacatttgcttatttccctgctgctcagaagcctttgatcaaggcaaaagagaatacaaacagcacatattactacgtttcccacctgaaaatgtttcctaacacgtggtctgatgtggtggtggaaaggagggaagaaccgaaaggttgcgtatagaggctgaagaaccatcgcgtcattcctgttttggaggtcacagactgctggctcctttctctgctgttccaacttccatgtcattataatcagtataatcagcgagtgctcctttacagaaaaagctaaccagcatttttaccagtacccatcaatatactttaaaagatgctaattatttttctattctctttataaagataaatattagtaatttttatttttgtcttactttggagtcaaacgaatgcagaggtcacaagcaaatgtgatcccagaacagttttgatctccaacatgcttaaagttttctgcccaggcacggtggctcacacctgtaatcccagcactttgggaggccaaagcaggaggatcacgcgaggccaggagtttgggaccagcctgggcaacatagggagaccgcctatctctgtagaataatcatcatcatcatcattagtaaaaaaagttttaaaattacttgtcaacacttaaaaaccagaagatttctatttttaaaaatacagacttggccgggtgtggtggctcatgcctgtgataccagcactttgggagggcaaggcgggtagatcacctgaggtcaggagttctagaccaccctggccaacatggcaaaaacccgtctctactaaaaatacaaaaattagccaggcatggtgatgcgtgcctgtaatcccagctacttgggagactgaggcaggagactcgcttgaacccggaaggcagaggttgcaatgagccaagattgcgccattgcactccagcctgggcgacaagagtgaaactttgtctcaaaaaaaaaaaagaaaaaaatacagacttacagtccgggtgcagtggctcactcctgtaatcccagcactttgggaggccgaagggggtggatcacttgaggccaggagttcaagaccagcctgggcaacatggcaaaaccccatctctactaaaaatacaaaaattagccaggcgtggtggcacatgcctgtaatcccagctactcgggagactgagagaggagaactgcctgaacctgggaggcagaggttgcaatgagctgagatcgtgccactacccttcagcctgggtgacagaacaagactctgtctcaaaataaataaataaataaataaataaataaataaataaataaataagaagtaaataaataaatagaataagaacttacagtaagaaaaaaaataaaacagtctgggagcattggaaatgactggtcagggctaaccagcagctgattcatccaggcaggaggcactctctgctttgctgtggccctgcctggtctgttttcctgtcccctagaggcatttgagttttcgacccctgccttttgaaggccttcccataaactgtgtgggtgactctttctgttaaaaccaggtatattggttatacctgggtgatgggatcacctgtacaccaaacttcacccatacaccaaacttcagtgacatacaatttacctgtgcaacaaacctgcatgtgtacccccgaacctaaaaattaaaaaaataaagtattagttagagaacaggctatgctgctgcaacaaaaagacccataaatacagggacccaacctgtgaggcattttcttctctctcagctattagtgcagagacagacatttgtggtaggcaggtagctctgctcgcaaagccatctagggacccagcttccttccttcttgccactctgcagtctgactgatgtccttgtccacatggtcagaatggcccccagcctgcaggaaggaaaagagggagggagaggagcagagagccccgattggagggtgagacaggaagttgcatggtcactgctgctcacatctttgtagctgcaagggaggcagggaaggctagtccccagctttgtggcccagtgcccagcccacagtgaaaggttctatcactaaaagcaaagggtgaaggtgtgccaggaaccatagttatttctcccatggggagcctctgtgctgtcatgaagttggggtgcccccagcccccaagcctgggcgtttctcagctgcttgaggagggccctgatctgaccccgtattgtgttagtcttagactggctggtgcagccgaaacctcgatggcttggtggcaatggctggctcctggatggccctgaggaggtgttacaaggtacctgccgggtaaagcccagaccctacatccaagcctcgatccctccttctcagctgcttccagaaagctggcagcaccaggccggctgtcccccatgcacgggcccttcagccaaacactcagccctctttgatcctccccaccccagcgaagaagggctgatggctgggggcttggccagaatcaaagcccagtacagaataagggcttcgactctccccaagaatggagcctcctcctggatggagaattgaaggggagggggcttagggttggttttcaaattcctagaattctagggggttcaccgctctctaatgatgaagcctccctgggggtgggggaggtaccaggaggcagagggcaatttctggaaggaacagcatgcaggtcgggggcctggaggcctaggaggtgcgagcgggcacccttgcacacctgcggctgtgcttctgactccacgtccatctgtgtgttgtgtgactgtgtccgctagagtgtgcggccgtatatgcaactgtgtggctgtccttgtgcctgtggctgaatatttgtgtgtgtgtgtgtgtgtgtgtgtgtgtgtgcatgcaaaagtgtgtgactgtattgtgagtgtacatgtgtgggtaagcgtgtgtgtatgtgggtgtggatatgtgtgtgcaggatttgtgtgtggggatgcgtgcgtatatgtctgtgtgtatttgtgcgtctgacttgcttgtgtaaatgtctgtgtccccatgcgactgtagggctgagtgtgtgtttcagggtgtatccgtgaacatgtctatatggatgttgctgagcctgtgagagtatgtttgggatcaggtgagcttggactgcctgtgtttgtgtgcgtgtttgtgggtgtgcacgtgtgccacatgccgctgagtgagacccagggcctagcctggaacctggggtacagaggcgaggccgcctctctgggtgaggtgcgttccctgcacccgtcccgggagtctgccttctagaccctagcatgtgcacctgtcctgggagtcggccttctagaccccagcgtgtgtccctcctgcagcagaggcgtgcagcaccacggaggatggggccgaacccctgctctgtccctcgggctatgagtgccacatcctgagcccaggtgacgtggccgaaggtatccccaaccgtgggcagtgcgtcaagcagcgccggcaagcaggtgagtgtggcaccccagccctgatcctggctcctgccccagtccccttctccctgtaagcgcctctcacccgcatggacgacctgccccaggaagcagccccagcacctgggcctgactgagagctcctcacatcttccccctgcccggctccttctttcatggcccctaccctttccctgtcacactagcatgtgaccgaagctcttgagccagcagactggctgcaaaccccagctctgcacctcccagctgggcgacactggggttaatttcttcctatctctgagcctcagtttcctcatctgtaaaatggggctactacatgctgttgtggggcgtgactgaaatcataagtgcagagcagtcggagcagccctaaggaatagtaggtggtcagtaagcactggatgttttatatccacgtatgtgtagatggaccccgacttacgatggtcaacttaagattgttagactttacgctggtgcagaagcaacatgcattctgtagaaactgtacttcaagttcccatccaaccattctgtttttcactttcagtatggtaatcaaattacacgacacattcaatattttattataaaataggctttgtgttagatgactttgcccaactctaggctaatgtaagtgttctgagcacagttaaggtaggctcggctaaggtatggtgttcagtaggttaactgtgttcagtgcattttgacttaagatattctcagcttgtagggggtatatcagtacataactccattgtaagtcaagaagcatctgtatctgttttcaaactgcattgaattagaattgtctgagaatccttaacttccagaacttctgccaggactgttcagtttgacttaatttgtgttcgtctttggacacaaaggcaataaatagatgagtgaattgtgttgctctcattggcataattttcccaagaaaagaaagaacaatggcacagcgattcctggtggtatctaaaaccatctcgaaccccactcaaggcaatcaagttgaaagacttcaatgtgctttttggcatggagtttggatgaactgtgtggtgagccaggaatctgggacttcagggtggccaggttcaccttgtcaaagtcagtgtcagtcagactgagaccacacccaggggcccctgtccccgcttcggcatctcatagccctaatagctaaggacaggaccctcagacaagattggtgataattacagtgaccctatttgccgaaagtatactggggagtgcatgatctgccaaaacctctttttttttctgatttgagaatgtttttctctgaagtgtttcaagaaaggcatagacgtaaaatcagtcatgcatttaatgtcattatcattgcagataaagtgatgcaaatcccggaatcctgaactttgaagaaattatctacaaaaggctttgaaacaaagagatgcctttttggtttcctctacattccacttgctatttgcagggctttgtttggggttatagggtggcttctccagccagatagcctgggctgccacagctactcagccccttactgctgtgggcagcaaataacataaccttttcaggcctcatttgccttttctgtaaaatgggttcataatacttgaggtacttcacagggctctttgaggagtgagttaatctgtgtagagtgtccatcaattcacaagcacagtaaacaaccttcactgttttatcactgcttgtctctagacactcaatcctgccgtgggtgggtggggtgtatggtcctttggaggtaaaaccgcaagggcctgccagccactgtgaggctgctggagtgtggaagggatagccttttccaaaagagagctgtggggcgagttgtgagttcgttgggctatgccaccatttataataatgacggcataataggaatgccttgggcacttgtctttttcatcccctgatcttttcattcagccctatgatgtctaagaccaaggtcaaaccaagtcaaactagttcttaagggttcaaaccagttaaaaaaattcccatttcaagacagagctaagcaaagagggtcagtcattcagtggtgaccacaccagtgatgcctactgttaacatgcacctttggagtgctctctttaaattaagctttgctgcaagcctgcaaaaggagaagtagaaccaggaggcagtgctgcataggtttcagagtgcaggctcagaaccacagaaagaagtccaaagcctgtaactttcttcctccgtggccttgagcaggtcacccaacctctcttagcctcagttccttgatctgtaaaatagagatggcgatggtaaccatagtgactacttcatggggtagttatggggcttgagtgagagaaacacgtatgaagtgcttagaatagcacctggcccactgtaggtcctcagtgaatgtgtgccaccatcatccctgtcttcgtaaagggaggatatgagttcaggaatttacccaaaagatgcagagctggaagagctggaattccaagactgtccaccagcctcagagcctgtgtgtgtgtgtgcgtgtgtgtgtgtgtgtgtgtgtgtgtgtgtgagcttcccctcccaaatgaacgatttgaggcttccaaatggaactgtaagtggaattccccctagccttgttctcagggggctgacacttagcaataattactggtaatttactggtaattatatataggtttgaaaacattttgaattccagcaacttcccttggagcaaaaatctcaggggaagggtggcccccttgtcccagccttaccaaggaagaagctttttatttaagtttttaaaaactgcatatttgaaataatttcagacttataaaaatgttgcaaaagtagtaccttgaatcccccacccagattcaccagttgttaaaaaactaccgttacattttcctttatgctctctccccacttataaatctacgtgtgtatttttctggaccatttgagagtaagcgacaggccctgtgccgacacctaagatgccatcctcccgtaagagaggcttctagcgggtaccaatagtccttcaagtttgaccatccagcacctggaaccaggcatttgaggggttcacattttatgcaagaggaaaatgaacactgacatgtatagtaaatctggtttttcagagagtgggttcagggaaaatcggtgcatttttctgagtaggtttgaggtcacacagaagctgagctgcgggggctggcccctccttgtcctgcccaagacactgcttgagaagaatgaagtggggcttgtctagccttgagtgggaggcaatttgagaaaagtgacgagtaatcatttttggtaataaattataagtgattatatttggcctgaggagggggtccggggtccacatttacctctgtctccttgtggtgttatgggccagaattcacccccaggtaaagggggagcaggaaagttttggggagctcccccagcctacaaaaaccttagccatttgctttggaaaatagttattgctgcagttgctgatcagcaggcgctgacccaccggagggcctggagcggttccatggagcgttagcaccttgaccgataaatgtccgtgctggtcccaaacagcctgatgatttttagtatggccacaaaacaaaagaaaaagaaaatgtctggctaccgcgcatatgtgcttgattaaccttatagcagaaaaggcagcgctgatcaccaggatggagaagtgagctaaatttgagagagattgccacattcaagcatccacatgtcctggtcacattcctgtcagcaaatgctttcctgctaccatggggtgggtagcacggctagtcatgggacatgcatgaaattgccagcagttgaccactttttacacacaaagatagaattttcatacggctcatcctaatactgcatagatatgttttaaatgaaattgtttcttttaatcctcacaataactttaagaggtactaattttatgcctatttttaggagaggaaactgaggctcaaagaggacgagtgaggattcatactcaagttactttgtcatttgcagtaatgacatcagagtgtctcttgggcagactcaaaagagtgtcatctcttccccctgtgaacatattttgtaatgacatttactcaacagtgtgttgaaatttggcttccagttcatctgttaagacaatggttttggccgggcatggcggctcacgcctgtaatcccagcactttggcaggctgaggtgggtgaatcacctgaggtcaggagttcgaggccagcctgaccaacatggcgataccccatctctactcaaaatacaaaattgactgggtgtggtggcgcatgcctgtaatcccagctactcgggaggctgaggcaggagaatcacttgaacccaggaggcagaggttacagtgagctgagatcatgccactgcactccagcctgggcaacagagtgagacttcgtctaaaagaaaaaaaaaaaagacagtggttctgaaactttttgctctcaagaccgctgtaccctgaaagatgttaaggagatctcacagggctttcctttatgggttgatggctctggatattatcatattagaaactagagctgagacattacaaatgctcatgtagcctctgaacaattccattgtacactagtgagatggggagactgaaaaacacaagtaattcctcagtgttattatgacaacagttttggccttctaaactcctaaaagaagttttttcctctcacttgttttccagatgggcgaatcctacgacacaaactttacaaagaatatccaggtaaaagaagaaactgttctttctttccagagggcacaaaaaaaaggcagtgaaagtttcttatgtgaagaaaaaaataaaagcccagggctgagatatagggaacaaaatgggacctgggattaagaaacaaaatagaagacctgtggtctgatggtgggcagggagttgtgagtcacagaggagctcatggcaaacatgacctcatgctgaagagcagagcctggaactgctcatatttaagcctctgctactttcattcagagaattggaaaacacgcctgataccactcttccattcatccatctaatcatccattgatccatccatctctccatctattatttcatccatccattcatccctccatccatctacccacccatccatctacccaccatccatccatccatccaccgattcatccatccatcctctcattcatccattcatatatccatctattctttcattcatcttcctgttttttcatccattcattcctctatcaatctattcatccatctattcattcttccatccatccacccattcatccatccatccacatattcatctatcttttcacccacccatctatccttttatccacccctttattccttcatccctccatccatccatcctttcatttacttacctattcaccaatcatccattcatctatccatccatccattcatacatacatcctttcacttatccatctatccacccatctaccttttttttttttttgagatggagtctcactctgttgcctaggctggggtgcactggagtaatcttggctcactgcaaactctgcctcctgggttcaagcagttctcctgcgtcagcctcccaaatagctgggattacaggtgtccgccaccatacctggataatttttgtatttttagtagagacgaggtttcaccttgttggccaggctggtcttaaactcctgacctcaggtgatctgcccgcctcggcctcccaaagtgctagatttacaggcatgagccactgtgcctggccccatccacccatctgtctatccatttatccatccatccactcatccatccacccacccatctatgcatccacccaccatccatccacccacccattcatctatccattcactcattcatccattcatatattcatctaatctttcatccatccattttttcaagcatgcatccatccatccattcctccatcgatctattcatccatctgttcattcttccatctatccatctatatattcatctatcttttcatctacccatctattattttatccacccttctattcctttatccctccatccaaccatccttatatccactcatctatttgcccgtacatccattcacccaaccatccatccatctgcccattcatccattcatgcacatatccttgcattcattcatctatctcttctttcatccattcatacatccatccatccacccatccacccatctacccatccatccattcacctatgtatccattcatccatccacccatatatccatctatctattcagccatccagccagccactatccatccatctatccatccatccatatatccatccgtccatccacccacccatctatccatccatccatccatccatccattcatctacccatctatccatctatccacccacccatatatccatgcatccatccatccatcatttatccattcacccattcacccatccatccatccatcatttatccattcacccatccatccatccattctcccatccatctgatatgtctttatcaataatttacagcacatattttctaagtattactcatcagtctgtgctgggggttggagctacatttgtgaacatgacagacccagtcgctgctgtcatgcagctaatatcaagcagaaaattcagacatgaatatagcccaagtggttattttatgagaatagtgacaagccttcctgacatcacatgctggatactatgaggacaccttagtccaggttgtgagaaaaggcttcctaaggaggagacatgaaagctcagcctaaagaataagaaatagtgggcagcagaatggcatgtgtgaagctcttgaggtcggaagcaatttggccatccccaaagctggagtgtgtggtgagctgtggaggtggtgggaggtaagaccagaaacatagacagagctggtgacaccagctggggggcctggggagggagaggaggcactgcaggtggggactgagtgacctcagctgggggagggaggagagggccaggtcaccaggcttcatttggcagttcctggtgagccacggggggcattagagcaggaatagatacatctaccccaacagagctgtgttcttttcacagaaggtgactcaaagaatgtggcagaacctggaaggggacaacagaagcactttcagtaaagcaacggcaagcaggtgagtgggcagagagcaagagtcatggccagggtaaatgtgggcagccagtgtcctggcagctttcaccaccaggttgaggagcaggagggtgagagtagcgctttccctactggtagaatttgaaattcccttctgcagtaagtcctcacctaacgtcatcaataggttcttggaaactgactttaaatgcaacgacatactatatgcactgtatgccatagaagcttaactcatttgtttgtttttttaagagatggggtctcactctgtcacccaggctggagtgcactggcacaatcattgttcactgcagcctcagactcctaggctcaagccatcctcctgcctcagctttagtctcagtagctgggactacagacatacaccacctcacctggctaattttttttttgtattttttgtagagatgagttctcactatgttactcaggctggtcttgaactcctggcctcaagcagtcctcccactttggcctcccaaagtgctgggattgcaggcatgagtcactgtgcctggccttaactcttgtttatatccattagcctatggtaaaattgtttttgttatatagtaggtcattttgcttaaagttgcagtttccaataagctatccatgatgttaaatgaggacctactgcaatttcaaggcaggaacgtaggagaactttgtgtgtgatcagaagcagaagcaattctgtatccctctgaatcaaaaatccaagaccagactaggtcgcaactttccttttcattcctttgccttgatgtctccaggaagtcttgtctatctcttctcatttctgtcaaacaattctgttgggtgccaactatctgatagtcatggttccagcctcttggttgctcagcggtagttcatgccagggtggtatagtgaacaaatcccacagagccgctgccctcagggaggccacatcctggtaggggacggctacacaagccaacagatcaatcaatacaaagacaggtgtggtgaagaagtgaacaggcaatgtggggcagagtccctgggcaggagcagagcctggggcaggggctgcttcgtttggggtcagggaaggccctgtgggggttgaagtctgagccaagatccaaggtgagaaggtgctgcttggggagggtctggaggataggcgcccaggtggagggagttggaagagtgaaagtactgaggcaggagggacgctggctttggggagccaaagaaggcccctggggtagacgcaggaaggtggcagggaggctgactccagaggaggccgcaggtgaggcacaggcttgccctcacgtgccctcaaaggtgaggcgtaggtgcatgcaaagggctgtggagttgcagccctaaacctgaaaatctacccagcccctgtgaatgctggatagccacagagggcaaaaaggagcccctaaaagcaggtgacagcctcccaggacgtcgctgaaacgcaaatgtcagattgcaggttccagaatcctcccggaaaatctggagcccgtagggactgatgccagcacgtctgttacctgctgatgacaggagccgtccctggagtaggtcagatgcagctgaggactgagcttctaacagggaagaaggaggccagtggatgaggacgagaccttgggaggcagttgaggatggcagtttggaagttgaggcttttgggtgggcgcagcagctcccgcctgtaatctcaacattttgggagaccgaagcaggcatatggtataatcccaggagttcaagcccagcctggcctggccaacatggtgaaaccctatctctacaaaaaaaaatacaaaactacccaggcatggtggtgtgcacctgtggtcccagctacttgggaggctgtggtgggaggatcacctgagcctagggaggttaaggctgcagtgagccatgtttgcaccattgcactccagcctgggtgacagagtgagaccctgtttcaagaaaaaaaaaaagtacagcctttggagcaagctaggtctgggtttaaattcttgttccagcactgtggatttagtaaataatttcggctctccaagcctcagcctcctcatctataaaatggaactgatggtaggggaggagttaggaactaaataagatgatgccgtgaagacgtcacagtgagttcagaattgggtcttctcagaaaggggcatcttcctggtgtttctgatggtggcggtggcggcggggagacgaaagatttgtgactctgtggtctgccctgcagtggttcaccctgacccttctttgtctctggtctgccctgcagcggttcaccctgatccttctttgcttgctttccagctaggttgcaagaacattcctctactttctgctaagccttggaaacagttgggaaaagtagtttgaccctcacagttcacattcagctcagcagagcaagaccccagagatgcttagagacaggacacctggccatcaaacccagtttggcccagcctggttgggtgactttgtgggagccacttaacagctctgggtccctgttttaccatcctgggagcaaggccctgcagctccacgagacctttaccccgggaagaagccgccacccatgaaagcatttctgaagcccctttctaagacaaggctcagcatcttgatatttttgacagattcctcccaagtctggctctgggaggtatgtacccatctcaaatgttcccaagataaattcatccttcaggaaatggaaatgaacttgcttactaatgtgtgattcctagttgtagccaccggatgtgctgaggcctaaatgttagcaggtgggaggaggccacagaacaataaaaacaaccaaataagatgccgagtctctggtgttttgtgatactttgtcactgtttaattcaggggtgggaagcagcgtgggtattgctgggatgaggtgtcagctaggctctgctcctggaaccctgtgttagcatagagcccctgaagcagaccttgggagggggatttgttgaaagtgattcattagggcggttcctgggaaaaactggcatgagagtggggaagcggaacaggaggagacagtcagacgagggagaggtgacgagggcccatggagggcacctttgctgcaatccctctgggggtcttggaggaagcctgagtcccaccagggctggctccagggctgtgcatccagaaggaccctatgcttggtttaatgatctgctgtcaccatttaaaaactcttagtcattgttgaacaaggggcctgcttgttcattttgcaaatgatgtcggtagccctgagccacactgtggagtcggtcccactggggctggacagcggaacccgagtatgcgtaacgcttccagcacccatcagtgggtggcgaggctgcccctccccctaagagaggaggcagcccacctgctgccagggagggagaggcac

SEQ ID NO: 9 - DNA, 87071 bp, Homo sapiens
  source 1..87071: mol_type = other DNA
atttcccaacatggtaccccaaagcctctcatgacacaatctgcctccctcattttggattttccaaaagattttactcccctgtcttcccagagaatgtgtcactccctctcattgtgcactgttcatgtttttaaaagagcacatgcccatcatatgactgtgaagttttcctctttgtctctgacattcagctgaaagcccctgaagggacttgaccatatcttattcctctttgttttcctagggatgttgtagttgggcacatagtagagctcacaaaatgttgctgaacatagtgcaccattgacacaacataattccagatttctaactttgatttctgaaaggtaaacattgctacaatgtattaaccacacagaagactcaaacaaacccaacccccttccaaacaaagcaggtttgtttgttttttgttagggcacccacttctacaaacaaatttctggccccaggataacacaaagagccagagtaggatttcaagcagaactgtgttttcagttgatgtgtcagtcccctgagagtcatgtggaaaaaaaaaaaaagaaaaaattcaaggtccaggttatttccaccactcctgggaaaccaggcctggagagctctctagggaaagaggtatgtctgctctgggcttttgcaaccttattttataattcactttcttatctactgcttcacaaaaccaaagggaaataggtacaaactgtatcgacaaaagatcagaactgaattctcaatggcaaaggcaagtgtacattataaatagcaaaacagctggcttggaccatgttgccggccagtcacccagttgagggatttgaatgacatcataaccctcgagagggtattgctagccagctggtgttatttagaatacacaaaaatcagagaaagaaaacacactctggcacacagactccctctgtcatacacacacacacacacacacacacacacacacacacacacaggtttgagttatatggaaaattcaaacaacaggaaaattgtttgccccccaggtacccttctcccagagtggtggggtggggaggggacagtgacaggcagcctagtagaagaataaagaaaaatgttctatttcagttgggttttacagctcggcatagtctttgcctcatcgcaggagaaaaagtatgagacagtgccctaaagggaccaatccaatgctgcctgcccctccataggttctaggaaatgagatcacacctctcacttggcaactgggacaaggggtcacccgagtgctgtcttccaatctactttaccccagtcacttcagggttaaaattgtagagtttgctggagagggtcttatcgtcctttctttctttttttgttttaaataatgcatttgctctagaatctaaaattgctctcccatcccccatattcctttaatactggtaaggtgtattagcagacgtttgtgtcttcatgcccagcagaaagttaatcagaaaacagatccttattttctatggcagcataagtattttaatgtctgcgaaccctgtcactaacacacattcttttaagggaaaaaaatgcttctgtgctctagttttaaaatgcaaaggtatgatgttatttgtcaccatgcccaaaaaagtccttactcaataactttgccagaagagggagagagagagaaggcaaatgttcccccagctgtttcctgtctacagtgtctgtgttttgtagataaatgtgaggattttctctaaatccctcttctgtttgctaaatctcactgtcactgctaaattcagagcagatagagcctgcgcaatggaataaagtcctcaaaattgaaatgtgacattgctctcaacatctcccatctctctggatttctttttgcttcattattcctgctaaccaattcattttcagactttgtacttcagaagcaatgggaaaaatcagcagtcttccaacccaattatttaagtgctgcttttgtgatttcttgaaggtaaatatttcttactctttgaagtcattggggaattctatttaaattgtgtactgtttgcttctgcctagaactgttcttcactttaaaattttcattgtttcggaaccgagagttatttataaattgctgaatatgcaattctgtggaatctgaaaaatagctcggggagatggatgcatttgcacagatatctgtatgagtagaaactattgcaaggtacttatgctaaatcctccacttctgcagggcttccgtggtgtcattacagaagattcctttaaatcctctctatggctaagggctatagagcatggatatgaacttggggattttttttttcttttgcaggtgcagatgttttatttttaagaccatgttcttttgcatgtgtgtatgtgtctctgtgtgtatgtgtctctgtgtgtgtaactcatcaggactttatttacaaggcatgctatatatgaagggttgtattttagatgatattaaagcttttaaatatgatctttggagctaaggtcccggaactctgcacttatgcccagagagagtgttaaagttagagtaaagtgtaatttgttcttccaaaaaagaactccttgacaactcttgctgaccttgcatatttgtataatttaaacaaatacatactgtatatggaaagcagaaactttctaagagctgcttgcccaacttttctgtttagaagaggactttcatgggcaaagtttggacttggggttctgtgttataaaactctgattttatattcagtgtcgtgaagtccctttaggtaaatctggctgctgctgtcagtgcaccgacttctcgtttccgattgctggccgtagttctagtttccattctcagcaaaattatatccttcaagacttgtgtttttttttcaatttgcaagcgcttttaagctgctgtcactggctccaccgattcaattgcctgagggctcaattcataagacgtctctgccacttagtgcagcattcagttgctgctttcaaacacttcaccactacgactttccctagtcaagtaagtggctccaggagttaaagatacacccttgtcaggcacctgattctgctgttcctgagatgccaacacatgcaggccaccttgctttcaaagaaatgacgtcactgtgcatacatactatgtgatctagcagctggagtttttgtctccttacttaggggatcataaaagaggctgtggagcgttatctctgcattaattacaagttaagaaaattgtttccaaatgcacttttcatgctgtgtatgctgaacacttcagaagtggagctcttaataagttgttacttttttctgtacttgaagcaggaagtggtttgagggagctgcgtggtctttcacatgtaattcagtgggtaaaggtgtcctgcccagaggcagagctcaccagctgattgtactctgactctcaaggtatttccaagtgagtgagtcgggggaagggagtaagggagtggactggagcttgggtcactatctgggctgctacaataggcatacaatggaaataggtggcttgactggggggaaaagattgacttaaatcccagctgtgcaatttgttcattgtttcaatggacaaaaggcagtttacccaggctcataatagcatacctgcctgggtgtccaaatgtaactagatgctttcacaaaccccacccacaaagcagcacatgtttttaagacttcagttttctattcacatcggcctcataatacccaccctgacctgctgtaaaagacctggaacaaacaaaaatgattacacctacagtgagtattttcttatgactgttgccctcaaattttacagggcattttcattgtggcccagacatctggaatcaattaatattccatttatctaagattaaaaaaaaaagaacttttaaaatttgggtttgtgaatgatttttgagaaagtgttttctgatttttttttctttttttctcatgtctttctgattcttcccttttttttctatcatatctttcctttctctctattgatttcttttgtgtttggcaaaataaaaggccaaggaaataatgaacatatgggaccacttgtttcacactttaaactcctaagcaagttcggtattgttttcatttgtgggaacataaaattctctggttctctgtgggtgtactggactgttccctacactaaaggaaaatgcactagaggttctgtcttgtcctagaactgtaagtactgagatttatctgacaatataactcagcaaaaaaaaaaaaaaagaaaaagagaaatctatttaaaaaaaatcttaaattcttaagttccagagacttatatgtatcgctgcacttaaatgtgttatactagaactattaaaaatatagttttgacatctaatgaagtttgaaattcactaaggctataacttgagtaatttttttccctggatttcacaaaaactgaagaacttgaaggcatctacagttgcaggtgcttagccttcagcattgctatgaacgcaacaaagaagggaggggtggagaaataggattcggtagaataacatactttctagaactggcccattcccgcagtgtgggggtatgctctgcaagatcaatcacaggtttgttcttttgaaaaatgccacgaggtccaggtgggttggatttgttgaggtttccaatggaaatattgaacaggaaaaccccactaattttacaaccaaaaaaaaaaaaaaatgagaactggatgataacatgcttcttccagtcccagtgttttgggctatgtagggggtggaagatttccatgcggagatcttgttgagaaaaatgcagtaaatgttcagttgagcttttacctcctcttctattttatgtccattatttatgatcatttgcaaatagagggcgcttttcatcttcaactgctttacaaccattaactgaatatttatattttgttaacgagagacaggggttttggttttttttttttttcctcagttgctgaggtgaaggtctgactacaaaaaatggaaatcatcattagaatgatggggaaaattttgccactgatatagagccactaaattgagagtccatggagggcaaggagggtagtcagaaaataaatgactgtttctccaaaagtgatggggttgaatggacctgccactactctgaggttcaaaaaaaatgctcttcttataattatagcttccagcatgactccacaacccttttaatagcgcccctcacctaacaccctgtcagggactcttcccatgatgctgaaagtagatgcatctgagagaagtgaagccactcaccttgagaggcttcttggaaaaagccacatctttgtgccacaagtaacaatatggaaaaacttaatgatcatcttagcatggtttctactacttagagtgaggatgaagacttgagccacaggactgctgcttttggcaaggctgccgtttggcagtctatcattgactaagtcatgccaaccaggggtcccttggtatattttgctataaaatagatacagtgacacataagtccctccaagagatgaagccataattcctgtgactaaaaataaaataaaagatttttgccagtttgagcccagtgccctatacacagaagggattctcaacacgtgttgatttgatattggacttgaggccagggactttagaatacaggagtggtttctatttgagccaatttcattcttcgattctcatatttttttctgtttctgttattttcttctatttgtggttgtgtcaactgctgatatgccccaggtaaggaatcagtctctctctctctctctctctctctctctctctcatattgaggaaacaaaacaaaaacaaaaccctgtgatgatatatagtattgtcaaaagccattttttggccatagtaaattatttttagtcaaaaatagggaatgatgttttggttaaaaatattctgctcaataatgaatgcatataccatctatggatggccaatattgagagaatttaaattaacaaaacttatttggccccaaatctgtactgagcacaatttaacctttgataattcaaaagttctaccagggtctcagagtgatatgggatcccactgtaataatagcatctttcatttccgtagtaaacgtttctagatattttgtctcaattcattgaaataggaacccataaagaaagggttcagggaggactcctccaaagatccacagtagccaggggaataaacacaggttgttggatgccgagacacgctccatccacaactccctctgggttctcatgtactctattggcttctgtgctgggtagtcctgattaatgacagtcgtggaatcgtgggagtcaatgcacttctgtcccaccccactccccttgcaaggatcaaggaggaaacctgaccctccctctgtttcttgggcaggtgaagatgcacaccatgtcctcctcgcatctcttctacctggcgctgtgcctgctcaccttcaccagctctgccacggctggaccggagacgctctgcggggctgagctggtggatgctcttcagttcgtgtgtggagacaggggcttttatttcagtaagtagccctccctctcaatgtgctgctcaagtctaaagtgtacagctctgtggatttacaaccgcagggagtgtgtgaataactgaatgagtgcccgtatctggcagccatcctaggcctctgagattcctcaccttaaagtaagcatagtgttttggcgggactttggcaggttttgcagatctaggatggaattttcctccttaacaattcgtctttaactacttcctgctaagccttctttcaactattatgatccacctcactattccattcctttcaatgaaaggacaggacaaaataaccctgaggtattgatgtttttgttttcaaaacctatgggcgcacatataaacatatggaacacatgcagggctgctttaagtgctatgtgatttggagtaaaatagtaaggactctccagtcatgtggatctgggaaaaaaaatattaaagggcatatcctagatgtctccattcaatatgaaatcaccatgaacagacagaaggttaactccccttaaaggcaggcctgtttgcaaatacacagactgtctctaaatgcaaactgaaatgccatactgatttgccctctatgccacttgaccttgatttttttgttgttgctctcttttggcatagttcttctccgggacccatcaatgatatcaccatctggaaaagggtctctgacaagtacagaatcacttatctcttaaatagctcttaggtgattagaaagtcaacagccagtaaaaagtagacagccaagtcaaggcgtataaataatacacacaagcaaaagtcaatgattcctgtgggaactgatcacttaaagaatctagctcccatgcatctatctcactgtatattataggacagagaaagatttaaagccagatgcttgttccttataattctttggagttttagagagttccaggaaactgagcatgagtcattcttaaaggttcatatcactccagtgtagacagcttacctcagttacccactatgctttgaatagctcctgtgatgactcacacacttgaagtctctctcatccttcccaaatgtcagcctctctctaggcaggtggggtatgtgtgtgtatgactggcagggtagagggtggcctgtgtataccagtctggagatcagtaagggagatgggaagaaagaggcttgtgagggtattggcgatcagtacttacagtggatttggcaacaaatagtttttaaatcactagtgacaaagaatcagaagtgccttctactatttgaataggcaaattcccactatttgcttccagccagtcctttaattgaacgaactcatttatttttctggcaataaagcaattctatccagttctctgttgcagagcagatgaatgagtgcattgggtgagtgattttccaatagcagacttgttttacctttaaaatatcctgtggaagataactgtgggacatcacatgatccaacagtgggtcataattattctataatttgcagctactgatcctcattttttaaagaaatgaaaaagtggttctcacttattatgcccaaggggactttttttttccttgataatccatttgtgcaatttcctttgatccagttcactaggtaattgccaagcctagaagtgtcctctgttacgtgccaggttgaattgtgatgttcaacacgacattgctccagaatttccatgcacagtgcatgaaaactggatctttctatgcctatgatgtttgcattaaatgatgaaaaattagaatggaaaaaatgattgttcctaagtaactgtatgatacttataagtgtttgcacagacacttaaggatattggacaactaaaatggaaaccaatttccaggttctcttattctgttactgcaacaatcaaatttcagcaaatttttaaaaattgtatctcaataacttgaaaaggaaatgttatatatagcaaatcttgattcttactatatgactttagctagaaagtggctttgtaaatcatatgttcttgtcagtattgtcattgaaaatatgtgtgtcatgagttttgccttatagaattccatgacttatgttaatccgacataccctcatgcacagaagtgtccaacataccttcatgcccaaaagtattcttgctaactttctagtcaactggacaatcttatttgcccaaaagaaattagtccatttagattattttcatgtaattcaaaacattgatggagcatctgttatgtgtaagcattgaatgagatatgaatgagatgcctagaaatgaatgagagattgaccttgttttcaaggggcttacagttgagtggtaaaagaaaaatgcaactaagtattattgtcataagagaaaaataaccatggagaataaattatatcctttatgtttttcttgaatattttggtctaccctgccaagaataataccctgtcaatgggcttcttgatgccaaaggacatatttatttacatttgaatccaagtgtctagctcatagcctggatgctatagtagctgcccatatttgattgttgaactgacaggattgaattgaattgaattgagtctagttgaatccaatcgaatagaatggaattacttgtttgggatcttggtcttaagaaccatcaagaattaaggttgtcaagtactgtggacaatacttctcacctcatggttatctagttatttaataaaactgcttttatttttttttgaactgaaaaaatatatactaacaatgacttagaggctgctgcattttggtcatccccaggctacattgatgtctgttttgtgtctgagccaattattcctacaggcatcaattgttgtcaacaaatttcattcagtttccttttcatccgctgagatttactgtgtgtggagaagtcagctctaagcacatagtctttgaaatactttttagagggaagcattcttccccacctctatacactgtcagttgactcctattattctctttacacttattttcaaagttttattgtggctgtgacttttattttaaactgcttcaaatccattttggaagtacaatttttttagtagtagaaggcagtttctgattttagtttgaaattcaaacatctaaaagaaaattgcctccatatctatcatttttgtcaatctgcaatatttaacaaggagagtttttttttaactctattagaactgttctgaataaaatattttgtaatgcttactgtgtgtagattgacccaatgatgtgaattgagctcatattccccttcaagcccaagttccactacagcaagtgaaagcaaccttctagattatctgtttgtgaacattatcccatcataaatggaacagttcatgtataattaatgcagcttccctagacattttatgaaatgaaagacccaatgaagagctaattactcattctttcctatgcatcatagggaagataggagagcctatcagccagccttgtgttcttgcagtcctgtgagaaaaatggtggcataatggagatgaaagcacattgggaaggagctagcgattttcttactagattgatggagcccttataaagggtggctcattacaacctcattttgcatgaaaaacaccaagagaaaaagagcttcaaatgaaataggagaagtgaagcttaccatagatataatttttgacagccatggtgattaaccattttaacatgctgctatgggcctgtggaatttctctccttggagattcttaaaaaattgacagacaaccaaccatcttggatggtttcaattaagagcaactgaaagaaagagagtttctggggtctttttaagttttaagagtctataaacagtcccttaaataaactgtatatgctttaccttggcggtctggggaaatggagaagtaaatcaattagaaaagccaagttggatattctttcagctcttcaggatttatagcttttgtaaaacatacttccaatgtatagttattaagaacaggtctgaataaacgaatggcattcttcatgggttaaaacagactgttttgtaagagctttaagtgaaactttacaatgaactttcattgttattgattgcaacaaccttcttagatctaggttattattattaagtcaagcttctttcctttgatatatactgcccagaaaactactcccagacctgtcgttcattagttgtaggagtttggcaggcatttaaacttcctgggtctaaacgtccttgtccatagaacaagggaattttattaaaataaatgacctcttctagctttaacagactcatagtctaggttgtatttaacaaagctctggtgctattcaagaaagaacaaccttggatttaagtagtgaccaaataacatttagtctcttttatcttatttacatagtttctgttggtccccagagataggagattccacccagttaaagtaagaggagccagattcttcgttttgttttgctttaagctggatttcaacccctcggggatggaatagaagcttagatatgaccaaatacatgcaggtcttttggactaaggagccaaagaagcacacgcttcatgaaaacagactgtcatttttctttgttaggatagacccactgacagttctcatctttacattctctcactctcctgactgtgaggtcaagtgagtatgtacatttttagatgccctttattaaattagaggggacaaataagaatctacaggtttctaaaccttcaccaaaagcctcttcccagcctattaccttttcaattaaagaagagagtgtataattggtgaatttgacatcacactcacccaccattagcattgttttgagtggggctggatttgagttgcatcattttcattccttttattggtaccccttagactgcctgctatgcatctgtgtgacagctggtcaaagttaggggtacagtcactagctgtgggaaagcttttctctttgaaactaattggtacatgtggggtttgaccttatgaccctggcctcattagtaccactaagctaataggctacagatattattaaagagaagcaatgtgtttttatattgaccccaaatgcttgttcctgtctttaaatagtaaaggaccagtttctgcaaccataggaccactaagaaaatgatgcaaaattagaaaataatttgcatttttttctgacatgctaatgcccttcaagaccatctccctcctgatggagagttataaacaggagagcgaagggttcaagaccaagggcatggcctctattgccaaacacttcaggtttgaaccccagctctgcaacttacttggtgagtgatcttgagcaagttatttaacctgtttgttcctcaattttctcatctgtaaaatggggcctgccttatagagttggtgttaggattaaattagttattatattggaatatgttaacttagaatattgcctggcacacagtaagcaccacatatgtgagctattacacttaattattctcttctgagatcaattgcagaatctattgccaatgctgaggtatatgaacggcccaattaattacacactcattttctctccttggcggcagctgattaaaaacctattatgagtagtaagaattatgtagtagtattagtaataatcacatgaagaatgttatgtttatctagtatatttactttttcaaaatccagttgtatcagctctctcactttgtcttgactccacttttggtgggcaaagattattgcttcatgggacaaatgagaaatgtgagacatgaacattttgtaacttaccccactgaatggttccagaaacaaagctaggttctcttgcctccaagaagtgtgtagcatagggagatgcttccaaaatcccagtagtcttctgggagataaggagcaagaatcccagggtgactaaattgattttgaacactttcttgaaaagaggcatgagagagatatctatgtaatgaccctatattttagcttctggaaaaattgaggaaattctaagaaaacttcctcaggatgcacaagaaaagtcttcttttagaagaggctaattagaggctgctgctgtacaagacccttcacagactgaattcagacagaacactgcaactcacctgtatacaccaggatatgcagtaggatctggggagcttctcttctgcccaacttcaaatcttcttcctcttcctgcaaacctcaagtgcttcattacttgcaactatttgtttaattggtctttctcaaagacctactaaaaaaatgctgtttaattggaccaaactttttcttggggtgctattagagtcgtagtaatgggaggtggtcacagacaccataatccaggcaggaagaaacttccccaaaataaaggagatgtgggagcagccagtggttgggatgcaagaccgccacctttgctggtattactcaacgagctgtttgaagaccattggtattgaaatgtatcctatctccagtggctggtaggggagacattttggatccaggagagaattttggcattctgctaattcaatgggcctccaatactttgaccagacatcttgtgtcgtgccatcattgcccaagatctccattagagctgacttcatctcttcagcagccttccttctggttttacagcccacaccacagattcagacacagtgaaaatagccacaaggtctctcccaaggagatgtctcattgttctcgataacagcctgcaagggctttgctcttttgtgcagccagacatctttgacaacccatggtggcaaaaaatcgttttcgcattgagctgccgccctggcagagttccatcgtacacctctccatgagacatcccaaagaattactttaggttgcctggaaagttcaaacccccaggccttattagctgttagactaaggagatgacaacacaacattttgtttgtctttatctatacatgagatcatgtttaaaacatagcaaagaggtgttatttgacaagcaattccctaactgcagtaagttgtatgcctatttaatgcattgtttagtctcacattggctaaagccacttaaagaagcacttggcaagctctagcttttcattcttggcatagctgcccctcttactttttcactgaaccttaaataccaagaaaaacccttcacttactttacatctggcatattgctttctgcaactggccagaacctttgcatgtggccctctgcatttgtcctaaggtaattaatggctttgcaatatttgatcatcagcagcacagacacaaagagaaattgtgcaaccagcagagaccaaacagcctcttttaaattaaaatcttttgcattaaatgctcaggcattctgcaggaataaagatttccatgccaaaaaaaagaggggtcaaaaagaggggaggaaatacccaagaacctgattattgattaagtatacatctctgtaatctctccctcttaaaactacccgattgttcaaagaaaataaaattgaagattgctacaaacttagaagtccttgaaaaataaacttgtcaatattatgcaatctagtcatgtatcaaaactgcacttttaccccatacattcatgtacatgaaaatgataataataagaattagccaaatgctgactgaaatccagagagaagtgacatgtgcaaagtcacacagctaattagtgacaaagggagaagatactgtgattctcttttctcatcccactgcagtattcctccatttagcaacaattagccatgtctgtgtcaaagttacctggtaaattaaaatgaccaagagaagccaccaatcaaattcatccaaatgagcatagatcttgttggttccttcctttcaataaattcacctgagacttaggttaacacttttgctccctcgctgtggaaatttgtacaaactaaagtgattccctgcctgtctcctatatggtgtgcaaagccttcacatgtactttgggcattccgtccacttaaggtcataatttgactattattgtatattatactattctgactctatggtcactctctccaatgtcaactttggtagaaatgctttggaatgacttagcaaagctgcgctcctggaaaatctgaaacagtctgacgagttgttggatcagctttttttttctggagatttctctgactgtcccaaaccaaaagaaatttacacctgatttttttgcagactcccaagcaaatactctatgaatctcttctggttttcttctttcatctctggtctttatgaggtactaaaaggctctttgagggacccttttaaactttgaaaatttgtcagacatagaagcaatccggagagtattgctgggaagatggtactacctctgtgccatttaccctgatgaaatgggtctttcatcagaggcaagtgatttagaaagtctgcactgagccaacccacatgcagccaactcctgattacctgtaaaactggggaaaggagcaccaagacttagaattctggattaaatggatttaaggacactatagatttttcttctccctccttgctgaacctatccccaacctctttatgagaccaagtgatgtgggttttttcattttggtttttgtttttttgagacagggtctcagtccattgctcaggctagagtgcagtggtgtgaacaaagctcactgcagcctcaacctcccaggctcaagtgatcctcctaccacctcagcctccccagtagctgggaccataggcgcatgccaccacagctggctaacttttgtatttttttgtagagatggggttttaccatgttgcccatgctggtctcaaattcctgggctcaagtgatctgcccgcctcagtctcccaaagtgctggatttacagtcatgagccactatgcccagactgttttaaaatcttaccttattctcatactcctcagtcaactttccccaagcgccattggcaacagagtttttcagcctcaacactattgacattttgggtcagactattttttgctgggagagggagctgccctgtgcattatacgataactagtaacatacttggcctctatccactaggtgccagcagtcccttcctcccagtcacgaccatcaagaatgtttgcagacattgccaaatgtacccctgggggcaaaatcacccccgagtcgagattcctagactagacaagtctccagaagagaggcaattagagagattggaaaaccatgagtgaaagtaacacatcaattctcagtcactgtttaatccagagcctatggtccattgcctggagcaagccatgagaagagtcattgtggttttgaaagcttttctctgaaaaataaaagttcttcctataggctatttaaaaccttttgcaggccttttctttcttgccttatagccatggaattcatgtattttagtaagagaaggaagggcaggcagaaatggttcagagtagaaccctgtgcttagaagtttgattgagaccacctggagcaaattaaaaagaagttggttatctttatttatttatttatttatttatttatttatttatttatttatttatttgaaacagagtcttgctctgttgcccaggctggagtacagtggtgcaatctcggctcactgcaacctctgtctcctaggttcaagcaattctcccgcctcagcctcccgagtagctgggatgacaggcatgcaccaccttgcccggctaatgtttgtatttttagtagagacggggtttcaccatgttggcctggttggtctcgaactcctgacctcaggtgatccgcctgccttggactcacaaagtgctgggattacaggcgtgagccaccgcacccagccagttgtctttgaccttaacatatgtggctgttcccttgtcaggcacataataaataaactcaattcctcacaagtgaaaaagcctggttcaaaggtaatgaccaaatttttctccttttttcttgtgcctttcctatctgagtcctatcacagtcattcaattgctcagaaacagtcagaagttccccatttgggcctgacccagctcccctactgaacagttctatgaccttggagaagtcacttaacgtccttgagccttacatagcttatcgtaaaatgggaataaaaatacacttctgagttgttctgaggctcagctgtagaagtgctttggaaacgaagggccatccaaatagtcacaactgatgaactgaattggatgctttttgccttgtatcccaaatcatggctcacaaattagatcaatgccatgatttctgtaaaaccttaggcaacaattataatctggaaaagttaatttggcagtgtatcttctcaacctcattaattaaaaaagaaaagcatgttgctgcctcctgttcacatatcactttacattgagaccacccaagagttaattaatgtatcttcagaatgctcactcagtatcactatttcctctcattggaagaagcagcaaagagatggggcctatcattttaccacactgagcttgcttaaagactttgttccccacgaggtgattgggtagtcactgagagatatcacatataggcaaaagatttctattctctcaagtcagctattggtgatttctgcttagcctcaaaaagcagcattcagcagaggaaatgtactcactaatcagaaaaagtatcttcgcactctcaagttgtacatccaaactgttttcttatcacagcctgacttaggggaattaaatttttttgtttgagaatggctttgatgaaatcaaaattgtgagttttatcaccatatggaccagtgaccctgtggttcttctatcccaggggcctcacatcccatggctcccagagacacaacatttgatgcagttgtaacacttttgaaccacacaatcaaatactggctactgctactgctgctactgtaaattttcattctctctattttatcttgagctttttcagaagaacacagactaagttttaagggttttcccccctcctgtttttcacagcatctagtatggagcacttaataaaatttcttgatttgatattgaaaatgtcattccaaaagctatttccttcttccagtccccagaatatataccatgacttacaatttgctactgacatcattttggatctgagattgaagatataggagaaaaaagtcaacaagtctacttgagatgtcacctcttaggaccccttcaccactgctccactgaaaatgcctagctcaagattaagctaactctagtcaacaggtaattttcaaaggctactggaccctcaccaataaggtctacttatgaaagtgagaacagaacctccactcagttcctctctacttctatgtgagttatttaaaaggagtagtgttgggcaagggaaggtatgcaacaactggtacatccctggctaaggtatgaatcacagctggagcaccagcccttagagaacactacaagtggtttctatttgccaatgcagaattgaaaaattagagagctagaaagaaccatatagagcaatctgatccagtgctctcacttcccagatgaaaggaccaaggtccagaaaaaacatgatgtgcatgaggtcacaaaagttggtcggaaaagctggagctactggtcggaaagccaaggctacatttcagtcacctcatgcttggtccagagctctttgcacagctggaccctttaaacttaacctcaataaactcatatgagggtagtgctcataaccacagtccatacactcaaagtgaactgctatgtcatattcttctggtgtaacacctcagaatagtttctagaaaattaagctcttagccattcatgaaatcttcatttactaatttctaccatccatcggataacatcatgaagtctctttatcccaaatctgatcgacctgactgtaggactgtgtaccttaataaaccctagtagtttgagggattggaggcttacaaaagaaataaaggaaaaagctgattttaatttgaagtcaagccagcatgttgcattgctctctaactcagaagtgaccccaaacaaaaggaattatcgatgtgttgtctgtcttcacacattcctaacttgctagtcttccacgaagatcttccatgaagaagcttaaattttcctacgtagtttgagttttatgtttctttccattaaagtttccagccgttcatagacatatttagtttggcagaaggtagtggtgactcacggaatgaagaaatttgaaggcaaagctactgaaaatggatctaatttctttttggggtaatttgggagcgagcaattgataatttgtctaattttcctggtttcatcagctgccacactgccaaatatatgttccatatggatgctttgttattcctgaaacatataacaaatttcagctgcgactttatctaatatggtcaaagggtaagactattgttggctcattttctcattgtttttggctggacaccctcaatcaatggctattgagtgaaaaaacaggatcatattgaaaccattttcagcatgttgcttcctatggatccaaccatttcactgtgagtttgtcaagcacagcaaatgttctctattaaagggatgactgtgttctcaacatggatgcgactgccagttaattgcattcaccttataattccagtctctcaatttggtgtagagatggttttggctctccaaagattccttcttttctgctgattcttacctcctagtatggaacagatatttgaggatgaattttgagaagatcaatgacacacatacaataagcatcatttcaggcttcccttaagttagacagtctgtgggccctgagtcatatgaatcataagacttgtgaaaggtattagtgccacaaatctggctggcatttctgtagattatcggtgctttgcatgcaattatgaaaatctctcttaccagataagtcagatgggggttcagtaacattgcacaaaagcatcatgatgtgtaagctaaagacaaagaaaggcttacttctggctggttctgaagtagacactggccacttagtgttttaaagtcattcagtggatgattggtttcaatgaatataaatgattttgatctccctccccaacacacatatcaaaaacaacagtaataataaagaacagaaaagtctatctcttagaaaaaccagatttgaaaactggctagggaaagtgttttgtggcaagaaacaatctcttgtatctcttgtttacaacgggctaattggtttgattaacaaattgtctaaggggaatgtatcaatgttgcttaggaaaaacaaagcttagtgtgtgaaaatacacatagacatagagtcttgctactcaaagaaacctgagggctttttagaaatgcagagtcaggggccctgaccccaaaccaattaaatcaaaatctgcattttaacccgatacccacaggatttggatgcgtgttaaggtttgagaagccccatctagagaatggcaggaagtaagaggtacttgctaattcgtggctcacatttgataagacagagtacaaaaagatcaactgtcactgcagatttcaaaaatgatgttaaggtcaaacctgcttgtggccattaacaatatatatgctgattcactggtatttggtccagattccctcaagttggtggaacctttcctcagaggaatgatagaactgctccaaggataaccccatggcctgaccttctgtgaactggaaattggaacttctaattgctgtagctgcaatttccaagcccatcactggggccaggaactgagccccagtccttaagatcttgccagtttctttttcaatagttacatttttcaagaacagccaagcaagagtgcttcaaaaaaagaaaaaaatctaattgcattaagtggacgaatgtgtgttttactcatatcaaatttcatcttttgttatgctcagccagattctaatattatttcttatgggtctgagttaattagttctatgagggttcccactgggattttagagaaatggttttaaggtaagaaaagtctattttggtcacctatcagaaggtgaagggtaaactttaaaactgcttttcaaagagatacagctggcttcgggtctttacgagggtcattctgaaactcaagttccccagcctgagcccattaatgaaggtcattaatgaaggccttgatatctgaagggtgtgcttcttcaaagggacattgtcccacagcagggcaggtaaagtgagagaggaccagtttttaacttgtatttcttgaaatgacaataaaaagaaaggggggcctttggattggagacttagaaaatcttggatttactcagaactctaccactaaataacttggtagatttgattattaatcccctgggtctcaaaacctgttgccattgaaacaatgatacgtaaatgaaccaagacattccacctttgaaatgtgataattgtgatctctcttcccagtttggcagggaaaaaaagtcactaatatagttttcacattgttttcacacatcttttacttgaactttaagaacatgccacttcttgtctatattcttgacaaggataagatagtattggacttagaccaaaactatatatttgatttgtttagatttaatgcatattattttaatacagtgatatccctgactcattggcactctacccattgaattaatgttgacaagataatctctaataccttttccagaatgtttactgctgttcatgaaaatctctaaaatcccttaagaaatctgacggatatggagattattgtttgtttctttgttagtattctctcattccttttgctgggtgctttagtaaaatgtatggaagtaaaattatagctctgcccaatttaattgtagaatcatcagctgggcacctgctctgtgtttagcaatacccaagggagaatgatgccatcccttcaaagaggtttctttgtggggggacccatgaaattccaatcatacaactgcaggcttgagtcagatgggaatagttctcccctatggtatcccagtccacaccatctattgagtgtcttctggttagccaaaactaatgatgaaagaaatgagtaaagcacagctctatgcacatggaattgatattctagagcaatgtcacaaaacgtttccactcccaccccacctgcatctagtccacagacatgttttgtttggtccacccaatatgtatttgttttaattttggaaatataaaaatcaagagattataagcaaaaacaaaagattaacaacttctcttatgaaaaaaatggtgcaatccagcatggctgagcctgcattcttgagtggcagtttttggtcacccttttatgtgaagtgtgtgcccttgcaggcttaatttattcattatatacctatctggctcagcatacattgacctatgccactcctgctccaaatatgcaagatctcgttggatattaactatacgcttgtctattttattacgttcaagacatatctattgcctttgttctatcacatagttaccagactaactgctgctggtttatataaataagacacaaacctcatctttatagaactgtatagccctccccaaacagagcaagggtacagctgagctagaatttaaaagacaatcaagtcattctggtgaaggaagaaggcagattttaaaaactgaacattcaaaaatttagacatcatcaaagggatatttcctcaagagcaagtggagtttttagccggtgaccttgtaaagcaggaaaggccaggttcccagccaatgcaatggatgggttgcacaatcaccctaagtgttgagcacagtggagaccatcaacccaactcttcagacaaaacaagctgtagccttacagtttgttttgcaccgagatccataacagctgttacctatttactggaagatccttccagtcccatcactatttagatcctcctcatcgcccaccaaaactattgcaacagcctcctccatgatcgtgctatgtgcaagactgcttgaggccagccgtcccacccatgagattaatctttccaaagcaattcttatcactttatcccttttctaaacaacatccagggaccattcatggctatgtaaattgagtgagtccaaactccccaatttgacagtaaggcattttacgtaaggtctctatctgactttcttaatgtatccctacagcagagctcacaaactggcaatctgtaagttgtatccagtcctcataagtgtttttgtgtagtgaatacagtgtttttactaaaatttgaattatctgtcagtatttactaaatgactctccacttcatattggcacatgctttctgctttaccaaaaaccccccaccactctctattgccttgctctggtctacttcacttgcctgcatgacttgctgtagtcattgagacattgacttgattgactcaccaagtttacaaatgaccccctacatattccaacatccgacctatgtgacttattaccacctattttattttcctggactttcctctcttcttctctcaaatgtcacctcctctgtaaaatcttctaccagatgtcaaccctctttcctctgagagagagaagcaataatattactcttacagcactttgtttttggtttttttttttttttttggtcagtgttcctaccatctgcttaactataagctctgcaaaggtgagggctatgttttattaatctctgtaagttcccacttactccctcatacactgcactatccatgtctgcatggaattaattgttattcgtttttatgtggccttacaggcctagattagttatgtgtgcctttaggaaggcagtaactcatttgaaatgtagacatggctgagtcggaaggcctccatggacttttcggtattttcaagtacagagtgagaagtgctgtatctaaaggaaactactaagggaatttaggtaaacagaacatgatcctttttgttggaagctggctgtgatctccagattaaaacactattgcatgcctcatgaggagcatcagcaaatcctttagtatgcacaacttgtgttagaattgcaggagtcagtctattcctgggggctgggctgagataatgaattgagtcaatgccaggtagagcacaaatacgctagaaaaaatgaaacagaaagaactccgtatgctgcattcaaaagtgtcaatttctaaagtttctgaataactagaaaggggctcgcattcctgtttgccctgcagaaggattgtcaacaagttacaagattatagaatcaaatctcaggcaccagaaatggatccaacttagtgaattttgactggaatgaccagttgcaaaatattgaccaagaagaagaaagtcattatttttcaatgtcattaaattcagcaatgttttatggataactgtgtacctagaattgtgctagaaaccaaggggaagctgaaacgaaaaagccagagcagaaaactaacgttcatgaacacccatcatgctcccaaaaagtaacatacaaatctgccttaaggttaccttcaatctggttaagaaaacaattcttacccatatgtcagaataatcatcataaacctatagaattaaagtaagtaataatgtataaaaataacatttttttctcttattcttcctggatatggcagatctcaaaatatgtttactgaatttccaaaagaatacaaaaaattttcatccttctctctatatatacagtcttgtgaaatcgtccactaaagtttaaaagtaggtggaatttacaccttcatgtactctctgtctgcctgggtccttggctaggtagaggcatggttgtgttctatagactgagagaggccagaaatactgggcaaacaagccgctttgactttgagttgatgaaaagcttaggaaaaaatccctccttttgtagagctgccaggccagtctttccaactcaagttcaatgatgcgtcaaaattctacctggagtcatgaggttcccctcccatcttatcctctgtatcccccggtccagataggatttgatgcagataacatgggcactgttggttgcttttcttttagcatgaggattcctcaaaacagagttttgttaataaattaagcagaatttgagtgaatttaagcagagtttgagaatttaagtgaatgtctgggtatatttgaataatatttattttgctatcagcatgacaacattggatatcaaaaagagttctgaatatgaggtgaggagacctggacaccctgaaaagccttgaattcgctggttggctgggttctgtcaggcaattctctcccttgtcaaatgggtacagtcagccctgcctgcctacccaaagcagtttttttagtaccaggagagataatacctgtggaattgccacaaaattcttagaaaatcaggacccgctaccagtttccagaggtaatttgagtgaagtgttgttagaattgagggagggctaggctagagtacatcctgaagttgaaagtgggttagccaagatgcattcatgccccttcctttggaacgtggggactgtcattcctttttgggtcatgtatgatttctgtggactggaattacaagggcaaatgtaggaattgctgtaatattggtcagtacttttcacattccgccatcccatgagtagggataagaggttaattaaatacatctctcgggcagcagaaatgatcaaggctgtccctggaacacagttgcttcaaaaagtaggcttttgtttcagggaagatctcccacagagaagcatggaacgccaaagagaactataaataggactatcatttatgtcaattataaatagatttgttcccttttgattagaagattaatgctagctgaaactctgtagttgagcagaaatgagggaaaaacttctgacaaaatattattacatgctaacagttagatttgataccttttcctccctgaagtattttctctgattttccctaaatgaagactttcttctcgtttgaatttatgtccagtttagtctttgaagtgcgtttgttcatacttcttagctcctcaggatctctagattagacaatacacacacacacacacacacacacacacacacacacacacacacaccttttttaaatggaaaaaattgaataagttgaatgtccgtgttaggaatttaaaagggggcaaaactttctcctcagatgctcctaagatggccactacgccctcctccaaaattctgctttctgtgagggagatgttttaccaaattaccatgtacatatggttccttagcaacccatattaacactgagcataatgaccaaagagataaaggaaaagatcagtagaggaagggccataagccatcgctgctgagcacctgcctgatatggagccatattttttcattctccaggaagcgatatccaatgggatcccaactctttacatatggttaaccaacttcttgtccttgattgtgttcatgacttgtctcttcccagggccagagagagaaatggcaacttcccatccaagctgactgccaggttcaacctgggaggagagattgcagggtgcctcagtcctctactggacatactagaatgctggtctcttccctgctaggatgacaggatggggaattttgcctgagtctgcccaacttgcctcatggaggcttttgatttcagctactgcaaactaagcattaaggaaaggggtgcaggtagagactggaagagagggtagatgcaatgagtggagacctctgtgtggaactcctcccccacttgaaatgccctattctggtttcgctgtgagatatttcaaaatgaacttaacttgtcaactgaaaatgtactgcctgacctgagatttctggaaaaacatgaatcagaggtttggaaagtctgggttctaactcataaagaaatatattgaatatttataaagatataacattcttagaactgtttctgtgtgtgacctctctcttccaagtggcattgctcaacagcccagctctcatcagaaggctgacattatgctgttgtgcaacttgcagccatagcaatgtcactttgttaactgcacttgctgtgcattcaactattgagaagccaaacagctttaagtttgagtcagaagttctaggcaggatcattcttgtgaaacctgaacccttagttcttctaaaaagtcaaaatacatttggatcttgcaaatcgagaatgtcagagccaatattttcctctttaatcaaaacccaggaatggcacggtgggtggctggggccatgtcctcccttcttccctttcccttcccccacacttccagccaagattctccaggcttgagagagctgatgtggaaaagcagctctggcccttgtgtgttcagccctgaacctctctgccaagcaccacattaatggtggacactaacgcattgtgatatagaggcatgagcctgaaatcaccaatttgtccccagactgataggtaaacagcaaataggaaagccttcttcttccctctcatccctgcattcccaaatatttggggctctatccaacagcctggatctgcagcaaacatttgcctctgactcaattgaccccttcatggcgaatcttacatccacattggataactctactggctcaatctatgaacaattttcaaaattgaggaaataaggcccttttccattcttaaataggctggcctaaatattcttgggagcttcatggagagtgtcatgccctggcttattcgcaaaggtggcaatggccttcagactcagctaagacaaacattggcacatgcaggtggccatcagatgttaatgtttatcttgttcctctgtattgctgttatctttcagggaaatctaggaatagcagaaagatgaatctttccttccccatggggcatgtacttccttctagggagtaggaagagaaaaccctctcattccctagacctcatgagttactctggagggttggtagtagccatggttaatggcaagcttcagaagggaagagcttccaaaaccttgccatgatgcctgtgaagtgctgtgagagtactcatgtttctctggaagtcacatcatgtgtttaaggtaggccttgtacagggaggattttgatagatggaaaaattctggacacagaaactaactcaatagaagagcacatagacagcaaagtaaggagtatgtttgcaggtgcatgagacatccagccagttgtttgggtgttaggctagtgcagagcagtgcaagataagcccagaaagataggttggcacatctatattgaagaattataatagctagcactttccaagctcttatatgcatgacactgttttaagcacttacatgtattaactcactgaatcaccaaacaactctgtgaaataggtactatcattatcttcactttacaagtgaggacactgagacacagagaagtacagaatgtgattaaaggcacatagattctaagtgcaacgaatccagggttttaactcaggcagcctactcaagcacgcatggtacctgacaaccttgacagcatgctctatagagcatacatttattctttgcttcttcttccatccccacatccctagccatggaaatgctgagtaacttttaaggccaaactcatctctttcctagcaatgcctcttccaacttcatcataagagaaacttatctttccctgttagaggcctctctttttttgtacatacagtaattttcccaaccttacttatattagagttaagaatgtgttcatctgtgaacttcttcagacaagaattggcatcatattcctctgtatgtcctcagaatctgggacagtgatttgcatgtagaaagtgctcaattaacattgcctgagtataattgaataccggaggccactaactgaggcctgaagaaaagtttcctaatcctaccatacctctttaagatcatctttccaaattcaaaacaaagcaaaacaaacaaacaaatgccaagctctatagggaactctcccactcctgaaaacctacatcctataaaaaacactttggaaaagcacaatgtagaccctcctaggccacacacctaacacattattccagctttagccttgcttgactgtaaaagaatgctaatggcagcagtgcttctaaaggcaaaagcacagccacacatgaatgatgtatctcactaagaactgtggggttccaggatttctgagaagactcctttcattctgataaagttctcagcttctcgacccccaaatcctaactccctgaggaagattcaaaaacacccaatttaattaaagcctttttttttctcatattgaagtcacaagttttcacagggtgcaacgtgttgctaacccagcaatttccagacaaggctgctcagcaaagcaggtgctgttttctttttagggcccatgtccttaaattgctttgctgctcagatgtctcaaaggtccaagtttctctggcagattccagcccaagcgctcctctttgacccagctctttctggctcagaagacttctttttcccataggtgcagttagccaataagaagagaatgtcttatctttttaaaaagtacacttccaatgtcttcattagttatcttcaaactcagagggtcaataatcacagcaagaggagcaccatgccttctggaaggatgtcaaataagcatgctgaatggcctacagttttgtaattggcaagacccccctaacacagattgagtgttacagatctgcaaagttcagtgctaaaatatgattgattagtgtgcaaatatttactgagcatctattttatgacaggctctataacagatgctagggatatgacatttgtatgagtccattctcacactgctataaagaactgtccaagactgggtaatttataaagaaaagaggtttaattgactcacagttccacatggcagcagaggcctcaggaaacttatcatggcagaagaagtagacatatctttttttttttttttttttttttttttttgagacggagtctcgctctgtcgcccaggctggagtgcagtggcgcgatctcggctcactgcaagctccgcctcccgggttcacgccattctcctgcctcagcctcccgagtagctgggactacaggcgcccgctaccacgcccggctaattttttgtatttttagtagagacggggtttcaccgtgttagccaggatggtcttgatcttctaacctcgtgatccgcccgcctcggcctcccaaagtgctgggattacaggcgtgagccaccgcgcccggccgaagtagacatatcttacatggtggcaggtgagagagtgtgaagagggaagagccccttctaaaaccattagatcgcatgagaactcactcaccatcatgagaacagcatgggggaaaccacccccatgctccaatcacctcccaccaggtccctccctccacacatgggaattacagttcaagatgagatttgggtggggacacagccaaaccctgtcaacactgaaccaaactgacagggttcctgcctcagatgcttagaggttagccagaaaggtgaacatggccagtgccaaacaatagcaaggaagggagagatccctgtgcactggaatcatctgagcggtgagctatacagtatattcctcaagttttctttccttcccaatggttggaatatctttctcatcgatctcccatcttcttttcactttctcttcttttcatcatctacagcatgcattgttacacacccactgttacaaaaactggtattagatttgttgtggcaagaaagcctcactgtccacaggcctacatcttagcaccttgatcttgagcttctagaagttctggcttcctggaagttcattacttactctgcgtaaacaaatgaaatagagagcaataaaatgcgctcagtaattctcacattatttgtttgagatatactgaaattatctacagaaaggagagcaatactctcttatccttgaagatcaaatgcatcaaagagttacaggttttgataaaaacttcattgatgaacttaattcctatccaggagtcaattggtctacgtagaagagtcctaagaatagaaaagctacactaggtgtgcagaaccattcttgaaattctgtatatgaaactgtgactcacttgatataactttagagtgtatgttttgacacctgcttattttcttaccagtagcctttctggttttccaaaatctttctccttgttatccactgacaccatcagactaacctactataacaggttaacagactaagcaactataactaatctactataaagatcttttaaagcttcttcaaggctgaacctctgtgactctgcattcctagatcccaaatcctatcttcaattaacagaggtttaggagaaatgacccaagatccttcctctgaaaacccgcccaaagtattattgaaatcttattcaaattggaaagaatgatttaaaactagagacagagatggaagtatattattgtattgacaaacagcatgtagatattgagaaaatgaaattaaatgtagcaagagaacttatttaagtctcccaaatgagtttgaaagaaaggcaaatgtgccctgtatctgtatttgccaacacagaaagagtaataataatcatctccagattagatgatgaaggccttagccttcagatggaaggcgaagaagagattgaaaaatctggaaatgcagcattttatcaagtcgaatttagtcccagagcaattgggcatccatttgtctccaagtggagcagtgtacgatggctgtttccattcgtggcagagagctgctggggccatcggagcaagagatgcttcacaaagaaagtggaaagcagcgccaaaaggcaaatacaattcctgtcacatccaaaatagttcatttagctctttatgctactctcttactctttgtcatggagcaatttctaggagagtcgcataaaagtagggagatttttagctttgagcagcaaataagtgaactgggcaaggagagcatgaaatttagtgaggaaaaagaaaaagaatgacaggggaataatataaaaaccctgggaagaaagcacttcccctgtagaaaatctactatatgaaaagtcactcctctaaaacagatatactcaggatggattttctccattcggaagagtccatcactttaataaaacacttttgaagtcttcatctcgtaacctcacgtttaacatgtctgagcacttcttgagaactcgccctctctctatgctactggatgagacgaaaggagaatccgtgagcccggtgttctttggaggcctctctacctacgcacatttgtgcttcacttctgccaagtgaatataatatgacaaggcttagaaagcccaggtgatccagagggcaagagtttgtgtcaagaatagaggtagaagaaccatcgaattatagggctgtaaaagaccttgaattcataaaatccctccacagagaaggggttaggattcaaccctagaggaatgtcagcacaggcctctctcaggatgtcccttctctgacatcctgagagaggcctgtgctgacattcctctagttggggggcgggatggggaacaaataactttactgccttcctgttctgcaaacagaaaaccgtagcaatggggtatcataccaatttttcattattattatactttaagttctgggaaacgtgcagaatgtgcaggtttcttacatagctatacatgtgccattgtggtttactgcacccatcaagccgtcacctacattaggtatttctcttaatgctatcccttcccttgcccccaccccccgactggccccactgtgtgatgttcccctccctgtgtccatgtgttctcattgttcaactcacacttatgagtgagaaaatgtggtgtttggttttccgttcctgtgttagtttgctgagaatggttttcagcttcatccatgtcccttcaaggaacacgaattcatcccttttcatggctgcatagtattctatggtgtatatgtgccacattttcttcttccagtctatcactgatgggcatttgggttggttccaagtctttcctattgtgaatagtgctgcaataaacatatatgtgcatgtgtctttatagtagaataatttataatcctttgggtatatacccagtaatgggattgctgggtcaaatggtatttctggttctagatccttgagaaatcgccacaccgtcttccacaatggttgaactaatttacactctcaccaacagtgaaaaagcatttctatttctccacatcctctccagcatctgttgtttcctgactttttaatgatcaccattctaactggcatgagatggtatctcattgtggttttgatttgcatttctctaatgaccagtgatgatgagcttttttttcatgtccgatggctgcatatacgtcttcttttgagaagtgtctgttcataccctttgcccactttctgatgagatttttttttttttttttttttttttttattatactctaagttttagggtacatgtgcacattgtgcaagttagttacatatgtatacatgtgccatgctggtacgctgcacccactaatgtgtcatctagcattaggtatatctcccaatgctatccctcccccctccccccaccccaccacagtccccagagtgtgatattccccttcctgtgtccatgtgatctcattgttcaattcccacctatgagtcagaatatgcggtgtttggttttttgttcttgcgatagtttactgagaatgatggtttccaatttcatccatgtccctacaaaggacatgaactcatcattttttatggctgcatagtattccatggtgtatgtgtgccacattttcttaatccagtctatcattgttggacatttgggttggttccaagtctttgctattgtgaatagtgccgcaataaacatacgtgtgcatgtgtctttatagcagcatgatttatagtcctttgggtatatacccagtaatgggatggctgggtcaaatggtatttctagttctagatccctgaggaatcgccacactgacttccacaatggttgactagtttacagtcccatgagattttttttttttgtaaatttgtttaagttccttatagattttggatattaccctttgtcagatggatagattgaaaaaattttctcccattctgtaggttgcctgttcactctgatgatagtttcttttgctgtgcagaagctctttagttcaattacatcccatttgtcagttttggcttttcttgccattgctattggtgttttagtcaggaagtgtttgtccatgcctatgtcctgaatggtattgcctaggttttcttctagagtttttatggttttaggtcttacattaaagtctttaatccatcttgagttaatttttgtataagatgtaaggaagcggtccagtttcaattttctgcatatggaaatgggctaaatgccctaattaaaggcacagactggcaaataggatagagtcaagacccattggtgctctgtattcaggacactcatctcacatgcaaagacgcacataggctcgaaataaagggttgaaggaatatttaccaagcaaataaaaagcaaaaacaagaagaggttgcaatcctagtctctgataaaacagactttaaaccaacaaagataaaaaaagacaaagaatgacataatggtaaagggatcaatgcaacaagaagagctaactatcctaaatatatatgcacccaatgcaggagcacccagattcataaagcaagtttttagagacctacaaagagacttagagtcccacacaataatgctgggagactttaactccccactgtcaatattaggcagatcaacgagacagaaaattagcaaggatattcaggacttgaactcagctctggatcaagcggacctgatagacatctacagaactctccaccgcaaatcaacagaatatacactcttctcagcaccatatcgcacttattctaaaattgaccacataattggaagtaaaacactccttagcaaatgaaaaagaatggaaatcataacaaacagtctctccaaccacagtgcaatcaaattagaactcaggatcaagaaactcactcaaaactgcacaagtacatggaaactgaacaacctgctcctgaatgaatactgggtaaataacaaaattaaggcagaaataaataagttcttagaaaccagtaagaaaaaaaagacaaaatgtaccagaatctctgggacacagctaaagcagtttttagagggaaatttacagcattaaatgcccacaggagaaagtgggaaagatctaaaatcgacacctgaacatcacaattaaaagaactagagaagcaagagcaaacaaattcgaaaactagcagaagacaagaaataactaagatcagagcagaactgaaagagatagagacatgagaaacccttcaaaaaattcaggaatctaggagctcattttttgaaagagcagcaaaatagatagaccgctagcaagactaataaagaagaaaagagagaagaatcaaatagatgcaataaaaaatgataaagggggtatcaccaccgatcccacagaaatacaaactaccatcagagaatactataaacacctctatgcaaagaaacaagaaaatctagaagaaatggataaattgctggacacatacaccctgccaagactaaaccaggaagaagttgaatccctgaatagaccaataacaagttctgaaattgaggcagtaattaatagcctaccaaccaaaaaaaaacccaggaccagatggatgcatagccgaattctaccagaggtacaaagaggagctggtaccattccgtctgaaactattccaaacaagagaaaaagagggactcctccctaactcatttaatgaggtcagcatcatcctgacaccataacttggcagagacacaacaaaaaaagaaaatttaaggccaatatccctgatgaacatcaatatgaaaattctcagtaaaatactggcaaactgaatccagcagcacatcaaaaagcttatccacgacgatcatccctgggatgcaaggctggttcaacataggcaaaccaataaacataatccatcacataaacagaaccaatgacaaaaaccacatgattatctcaagagatgcagaaaaggccttcaataaaattcaacagcccttcatgctaaaaactctcaataaactaggtattgatggaacatatctcaaaataataagagctgttcatgacaaacccacagccaatatcatactgagtgggcaaaagctggaagcactccctttgaaaaccagcacaggacaagaatgccccctctccccactcctattcaacacagcattggaagttctggccagggcaatcaggaaagagaaagaaatagttttcaaataggaagaaaggaagtcaaattgtctctgtttgcagatgacatgattgtatatttagaaaaccccatcatctcagccccaaatctccttaagctgataagcaacttcagcaaagtctcaggatgcaaaatcaatgtggaaaaatcacaagcattcttatacaccaataacagacaaacagagagccaaatcatgagtgaactccattcacaattgctacaaagagaataaaatacctaggaatccaacttacaagggatgtgaaggacctcttcaaggagaactacaaaccactgctcaaggtaatcagagaggacacaaacaaatggaaaaacattccatgctcatggataggaagaatcaatatcatgaaaattgccatactgcccaaagtaatttatagattcaatgctattcccatcaaggtaccattgactttcttcacagaattagaaaaaaccactttaaatttcatatggaaccaaaaaagagcctggatagccaagacaatcctaagcaaaaggaacaaagctggaggcatcacgctacctgacttcaaactatactacaagtctacggtaaccaaaacagcatggtactggtacctaaacagatatatcgaccaatggaacagaacagaggcctcagaaataacgctacacatctacaaccacctgaactttgacaaaccggacaaaaacaagcaatggggaaaggattccctatttaataaatgatgttggggtatcataccaatttatgagatggtcatttggctcttgagtggaggccacctctcagtgactcagcaaaatttgtcctctgcacggtccttgtatcttgtaccacctgaggcaatcaagtatattctgctttatatcatagttatttatgagttaacgcatctccttactgggtcccaaattccttgaaggttgcaactttgccttgcctattcattttcacatactacgcggcacttagggcagtgctttcaatgtagcaggtaaccaattgttttgcttgcttttaaagtagttgtttttaaattgacttgagcttagttctgcaagaagactagatgagatatgattttgtcatcataaatttaaaatgtccttaatattttcttaatatttaatctataaagagtattcctggcacatagtagacagtaaataactttgttgaatgaacttacgcgtaaatacatcaatgactgactgctcatggagggtgccccctttttgcccatgaagttgaggtggtattgggagaatgctttcatttcctgatcctaactctcctgttgcagtctagtattgtcaatacaaaggtgtcatcattaccgttaatcccaaactgatgtaatctgaacttttgctctgtgtaattacagatatggggtacatttcctagaaaagaactgccctggacaaaactgttaggtagaaaaaaaaaaaaggaaattgggtgtcttttttccatattgtcaagagcagagaggacctcactctgccccctccctcttgactcctcacacccaatagcagcaccaatgcctgccacagaattatgagcacttcagccgggcgtggtggctcatgcctgtaatctcagcactttgagaggctgaggcaggcggatcacttgaggtcaggagttcgagaccagcctggccaacatggtgaaaccccatctttactaaaatacaacaattagccagacatggtggcaggcacctgtaatctcagctatttaggaggttgaggcaggagaatcacttgaaccccgggaggcagaggttgcagttagccaagattgtgtatcattacattccagcctaggtgacagagtgagacgctgtctcaaaaaataaataaataaataaataaaaataaaaagttacaggcacctcaaggttgcctgttttttgcctactgttgcttcagttatgtactctcagtgatctgttaccttcctttccagaaactctgtgtaaatatatcttaattcaatgaacaaagccataatggagacattggaggcattagtagccagccagtgatctgttgttttgttcattcaagaggaggttatcatgaaaatactctgtgagaaacagagttccaggattacactggcttaaacaatatggaagtttgtttctcttatgtagtagtcccaagatacatagttcagggctgctgaaccatctgctgtgcaaagtagctctatgccacaaaatcttcaagacccagcaatttctagatttctatttcaccatcccagcatggtggccctcatctgcaggggccaagatggctgctggaggttcaacattatacccatgctctagccaataggaagatagaaaaagcaaaggatgcattttggttgtcttttaatgaaggatcccagaagctttcatgtgacactttcacttatatttcatttcctagaacttgaacacatagccacacagagttctaagttaggctggaaaatacagttagctgaaaattagggtttctattactgcagaaagagggaaaatagctattgggaaattactaggaatctctgccaaagacgctatccctgagctccttacgttctaccacttgagtgccccaaatcaagacactacaaaatgaaaggttgatctagtgtcagccagatctagatgccagtcctattagcatcacttaatgagctttgtgatctctgagccattttccatgctatcaaaaacagcaataacacctccctattaggttcctgtaggatgatataagataaccagtggtgcctagcacagtgcctggcatatatggtagatacttagtcaacatttatttttaccattctttctgcatataagactggctcagctccacagtttatatcaacttgggcacaaaaacattaacaaagttcctggcggccatatttcttgagcaattttatactctatccattagccagatgtgttagagcaaatgagcggctgctcaaaagaatgtctttatcctcacttgcctttttttcctgttattgttggatggtgccaaacaagacagaagcaattaataaacttgtcagtatcataagagatgacaattagaatcagcagagcttaaaaaggagcctgaaatcataggggttgaggtagtggggtttttaaaatgcaaagagcatgccttttcacttcactctgtttatcaggccaagcaaactaaagtgaaaagtcactgtggaaaaaggtgaagaaaaaggtacctattttgcatcacccactatgtcatatcaagtaaatgaattaaaatcccatgtcaagtactgggctttattccccatggtttcacaccaagtttctcttagaagatactctaagatgcactaagctttcccattcacttctgtctaggcaggaagattctactcaagttaagaagcagtgttgccagatgcagtggctcactcctgaaattccagcaatttgagaggccaaggcaggaaaattgcttgagctgaggagttcgagaccagcctgagcagcatagtgagaccctgtctctacaaataatcaaaaattaaccagacatggtaatgcatgcctgcagtcccagctactcagaaggctgaggtgggaggatctcttgagcctgagaggttgacgctgtagtgagccacaattgcaccactgcactccagcctgggcaacagtgcaaaaccctgtctcaaaaaaaaaaaagaagaagaagaagaagaagaagaagaagaagaagaagaaggagaagaagcagtgtaatagaatgacttagtgctcagaaagtcttgagtttggatcctggtttgggcatttaataagtgagtggcctgagacaaataaataaacttttctatggctcaatattttcaactgtaaagtgaagatgataacagtgtccatctcataggattgttgtaaaaggtaaatgatacagcatatgtataaaccagatgcactcaggttttccagtctccttgggtcacgcttttctccaatatctacataaaataacctgcaaccccttttcacattccatgggaagcccatcctctgcccacgagctctgaagatttggggtatcatgcttctctcaagttatttcactctatagcctgcttcccaaatatggcccactcccacttgtcattcctgaaaccattattctatgttccctgccccctttgcttcagctcatctaaagggcaggaaaacgctggaagcaggagagtcatgagtgcctgaaattttgcaggagcagcaggcagtaagagtgggaggacctgagatctatgatagtgaaagacatgatgcaatatattgcctgcctggttaaaggagtagaaaggagtcaaagatgactgtaacattttgagtctgagtaagcaagaagataatggtgcagataccagcaagtccagaggaggatttgactttgttgacttgactttggttgacattgactttggtcaatggcaaagattgactttggtttcagagacactgatgtataatagagcttttagaaaataattcccatgagtcactagggtgcctgtccaaagtgcagtgaagggactgcatagaagaagaggcttggagatttccttgtacaggggatagctgaagcatattccccaacacaatattaaatttgtaaattacctttaaaaatagaaagaaaattgccattttagagtctcaaaaggactgagcaccatctaattacatcacaaatgatctcccaaaaaactcattcctttagaccaggagatgagtttctattcattattatttttgtagctaaaatttcaaatcacagaatcataatatattgcagtttgtttaagccttaaaatgttcctggcccaaatgcctcacttaaatacggggaaactgacatgctgaagagtctgagtgacctatccttgttcactcaagttgaataatgctgccagaaattctactgttagaatttctgctctcccttgctcattttttcacctgcagtccagctaccaaagataggattttagacattctatctacatttaaaatgttccttctaactgcagcggaaacacaggtagcaaggagacagtgtagaataaggagccagaggcctggattttagtcccgtggaaaaagaaccagccgcactacctggagagaatcatttggtcttccaagttctcagtttcctgatttgagtagcaaggagatgggtctggattcgctctgaaatgtcttgcagctctgacattccatgattcttggagaaatcggctatctccaactgaatccacatcttccaatcccacacaagatggagaagcaggcccatcccagtatttggggaggcccatcaggaccaaagctgtcctctctctgtgggggctggagaagggaagcagctgaggcaacacaaccagtttccttcccattcagaagagaacaattgagctcctcccaggggaagccatgcggatttggacagtgtggggtctgtcttatgacaggaatggaggatttctgtagaatggggatgcttagcggccgtaagaatcaaagatggaaggaaagccaagaggtccttaggtccggtcccttgcctctagctaagccttccaaggcagatggttgtcatcctacatttaaagatctccagagtagcaattgctcagcttcccacagcgcctggggtcagcaagtttccccttataatcaagaaattcttcccgatgtttaagtggaaatgcttccaatgtggcccgagtctgtttacccttgtctagtcctcagaagagatgaggaataatcttctgtgtaataatcactcttcttatgcctggtgctggtgattaagtcacttcttagccttttcttttttgggctagatcttaacttcattttccacccagttaatctttttttttttttttccttctcctagctggatcttttccaggttctccaaatgagccttaaaataggagaatgaaatctgagttcccactctaattaaaacctcagcaatgccaagttttgtggaaagattactctctaggctttcgtttcataattctctacatacacccgcctcatattacaatttgttgtagtactttcctgctcactcagggcccacagctattttgttatagtacttgtctctgccagatggagcccatttttgtctattttaaacatctttctgtttttaatgacttctcgtttctaaattgttaaaggtctccttaaattcaaatcctaccttctaatgtttctttacctgacttagtgccatctgaaaaacattctagtccattactcatcattgaggaaggaaactcaattttgtatcaatccgagaagggcctgaatagcactcatttctcccaatactcacaagcatgctttgagcatgaaaaaaaatgcaggccaccccttctccattcatttattcatgaaacatcttttgaacagggtagacactggacacacagtgatgaacagtttctgtatccattcactaatgtgttcctacatgtgtacaaaatgtactccttaagcacttactctgtgtaaaaccgttgcaagtgttgtggctacagcagtgaatagggaataaaaggctactggcttcatagatcccacattccaatgggtatgggggaggacagagaaaaattacaagtcaagcaacaaataaacaagaccatgtcaaatttgataaacgttttgaaagaaataataggatgatgaacctgaggatggtactgcctttgatggattggatgggggaggtccctctcagagggagaccctgaagacctttcagtttagcagagttatgttattctgaaccaatcccataaagcaaacccacttccctcatgttttcttctgacagacccagcactaactttttctggggtgttcacggtttataatgccttgttactcagtaattgtttacattcccaaaggactctctcatgttctttaattgaaagaagagataaaagaaggaatttaagtatacaggcgcctttgttcctcaggaagttaatggacattcctggaaaatgagagtccggtggtctattgtgtggagaaagtggacttcagtaccatgaaacagatttcttcactgtggaacttgaataggctgttcctcgaccatcttgaataggctggtgaactttaagactctgcaaaataagataacgtttacagccgtttcctaagctttttgcacggaacttcttgaacaactctttggtgaatgctgagctgaaaagatctcttggatccttaggtgtcctttgacataggatttggcatttgggatgtgattccaaatcaccagcagccaatgtgcaggccagtcgtcgatgtctctgaagagtgtgatagaaagaagaccgcagaggaaggcagggagtccttcctgactttgcctcagcttctcttgtgaccctggtcacgtgtgaactatgcttctttagttttctcagatcatggaatcttctgagaatcttctgagtgtctaaatgtcctctttccaggaaaatccacaaacatacataattatacatcatgcagagggttgatatgcctcctaccaaaatgcatctctgaactggaggatttctaaggatctgtctgtccagtgtgaaagttcaaataggtttctgctctggtcatattgacatcttggtgttttggtttgtatgcttgcttttaattgacacataataattgtaaataattatggggtacacatgatgttctgatacatgcatacattgtgtaaaagtctaacagggtatttagcatatcgatcacctcatgcctttatcatttctttgtggtgagaacattcaatatcctcttttttagctattttgaaatatacaatacgatattgttaaccagagtcaccctattgtgcaatagaacaccagaaattattcctcttgtctaactgtaactttgtacccattgaacagtttctatctccccacctaactaatgctccgcagcctctagtgaccactattctactctctacttctattagattaacttatttagattccatatacaagtgagatcatatagtatttgtcttttgacattagtttggacaagaccccaaaagcaaaagcaacaaaagcaaaaatagacaaatgggattacatcaaactaaaaagcttctgcacagcaaaggaacaattaacagagtgaaaaatcaatcttggtccatgtttttgattagcttgtgtgctccttgggacaaaaccaagcatcctaccacctctcaagcttccttcagccttcaccacacaagtggtgctaatatgtgtttctgcacctgtgccaggttcctctgcagagtcccgaggtggccgttgcatgagagactgctagggaggccagggagacccctccacacagaggtaccagccagccctggggatgccccgttgacttctcaatgagtgaatgaggtcatttttcctctgtcttttccagaaatagattcgttgagaaggcatatgctgaaatgacctagcaaggcagcaggaaggaagtgactagaaagagaatcattaaaaacccaacttcttatttaaaggaaaatggaaacaggggcctggaggaatgctctaattgggaagccagcagtggcccctgccttccctcccagtgctcccaatatggatttcctgcctttgggcactgttggatttaaagctatgaaagcctcatacacagtctcatcagaagggctcagaaaagaaacagagcttcatgtgccttcttcctgccacttatgtatggaatcagttggattaattaaccctgtttcctcagtgagattgattctatcactcgctcgagaaattctgatgattaggtgtaacctaactgataaataaaaaattataattttttttttgcaaaattaaatgtcctctgctttgtataattactgtggttctatgggcaatcatcagtggtatattcccttccaccctcctttgccacaaattttgaatgatccatgaaaagatattttgatgatgtggtactgaaggtgccccaaacccacactgcctttttggcttctctccattaccctaggaggttatagggcaaaggcataggaaggataggtttgggttcctgggaagtctgtcttctgagctgaattttacaattgagcaagttggcttgactggccatctgacaatctggaaagcttaaaaataaattgaactcagtatttagaaaaggcagctttcaatatcagaagactagcttgtgtttctcgctttccatttggcgggggaatctgtgaaggctctaatttcctttcctgagacctggcttgttgtcttcaggtgctagctggaattcaaaaggacacagactgtgcaaataaaaaactcagggaagggaaaaaggtcaacatgtgttttttgtttcagttcagtttgttactttgaagggttataatattttttccttctgttttgcaataaaactggttctgctactagactgacatatagatacctttattaggcaattatcacaagagatatgaaggctttctgtgtaagaaagtataagaggtctttcccaggactgcagttcttcccttctctccagtgccacaggacgtgatcgtatattatatacacacatattaggagttgatattgcacacaggcactggcatcatttgagaacttatggaaaatgcaagtgcttgagcaccaccccagtacctgttgaaacagcaactctaaaagtggggcctgttgatcgtgttttaataagccatccaggtgataaacatgcaggctcaagtttgaaaaccactagtatgtgttagttatcttgggcactacctgcttcaggaacccccacagaattatgggcctgaatttcatcctttcatgaggtttggggacaagtatgtgggtggagaagtcttgccatttgaaatcaaaggaacttccagattcataagccacttaaactctcagttttttcatttataaaagtgagaataattccattggcaccataaggttgttgaaatggctgatgcacaaaaggtatgtgaaaatgatttttgaactgccaggcactctctggaattagctatatggattgatgatgccaatacacattatggaccacaggcatgctgggggatgtggggagactgtgtaacctgcgtatcctgtgtcctcttctatattctgagcaccaagcccaggcagataatggccattcactgagtctttgctgagtaattttaactaatattgggtaagtgttcactcatgcatgcatcacaagcaccttgtgaggtgagtatgtgcctctttttggatgcaacctgagaggttaaggagcttcctcacatcacatagttagaaatggtatgctcaaggctcttctttaaaatggcatggtattaatacttgcatgtaacctatgcacttttcttgtgcactttaaaatcatctctggatcacttataacatctaatataatgtaaattctatgtaaacacctgttatactgtattttaaaaatgtgtattatttttaattgttatattgttatttttaattttcgttcaaatattttctatccatggttgtggacatgaaacctgcaaatacagagaagacatttatgcagccaaaaaatacatgaaaaaatgctcatcatcactggccatcagagaaatgcaaatcaaaaccactatgagataccatctcacaccagttagaatggcaatcattaaaaagtcaggaaacaacaggtgctggagaggatgtggagaaataggaacacttttacactgttggtgggactgtaaactagttcaaccattgtggaagtcagtgtggcgattcctcagggatctagaactagaaataccatttgacccagccatcccattacttatatacccaaatgattataaatcatgctgctataaagacacatgcacacgtatgtttattgcggcattattcacaatagcaaagacttggaaccaacccaaatgtccaacaatgatagactggattaagaaaatgtggcacatatacaccatggaatactatgcagccataaaaaatgatgagttcatgtcctttgtagggacatggatgaaattggaaaccatcattctcagtaaactatcgcagggacagaaaaccaaacaccgcatgttctcattcataggtgggaattgaacaatgagatcacatggtcacaggaaggggaacatcacactctggggactgttgtggggtggggggaggggggagggatagcattgggagatatacctaatgctagatgacgagttagtgggtgcagcgcaccagcatggcacatgtatacatatgtaactaacctgcacaatgtgcacatgtaccctaaaacttaaagtataataaaaaaaaaaaaaaagagatgactgtacttttgaaatgccctctgaactcaaatgatgaaacaagtgaaatcaaaaaatttgtttttcaagtaaaaaaagaaatggtttagccagcatctgaacccaggcaatctcctccagagcctgtgttctaagcacttcaccacacaacgtcacaatcagttcttcataatgatgctaaggagggtggggtgagatgacattctgggagtgcttgctctgtgccacaagctgtctcaagctctctttgtgccttaactcacaaaaaccctgtagagggaaatgatgatcatcctcttttaacaagaaaggaaatggagacttgagactggccctggtgttaggtttcttctttccaaagtacaaatttcttctattgcttctctctttgtaccgtttgtgcacccaacttgtcaatccaccaatcagtgagcatttgtgaagtgtccaaatgcccagtgtagtgcttctaaacacagaaggatgagatgacagtgtctgccctcgagatgctcaaaatcaaataggaaagaataggccagtcttcacaaaacagaaaaaaaaggctaatatacagttaagagcttgtttatgatacataaagtaatcattttccatatttagaaggttaatgggaatgaggtcatcacacaagaattctcatgggggcctgaagaaattttgtgttgctagactgagacttcaacaatttctctgagcaattatccttgaactgttgagttcagcatccctgcattgcagtttcctttaagtttgttacagttaccctgattataaggagatagtgacctgttatcctgtttttttctgtgttgttaacttcttatccctggtactgagaatagcgtctacaattcagtaggtgctcaagtttacatgtgaccaaatgtttataaaaaggagaataataatgtggtgaaagaaggacaggagtgttcagccaaattttggattggatgagaaggcagattgtcagggatatcatgacccaaacaaaggtggagagacaccagattagggaggagccaggttctccagagaggacctgagttcagaaggagcacaggagcagagcctttgttatttaggatgcaaactctcccactcccatacactgaagtgtggtgctgtcttggcagctgtgtgttgaacctctctccctaccccagagaagaaacctcatacgtacttggtgattttctttttaagttttttatttttatttatttgtttatgtatttatgtatgtatgtatgtatgtatgtatgtatttttattatactttaagttttagggtacatgtgcacaacgtgcaggttagttacatatgtatacaagtacttggtgattttcaaagttgagagaccagagtcccttgtcatcaatttcattttccttggttttacttctacttagaaaacctttggatgccaaactcaagtcttggttacgccccatgtttccttcaaaggaaactgcactagtaattgaaaacagaaattggttcatttattctaactcacaggacacaggtaagatccctgtctgtgctccaccttggctgtctcttttctaatcccgggagaaaatctggatataattctctgctttgttgattaaattcctctggtccttatgcaaaattgtgggctttcatcaaaataggattgtctgataataccctttgactcagtaattccattccttggaatttattcttataaatgatctatgagtagtagaatgttatgtgtacaaagacatttattgctgcattagttaaaatgatgataaaactagaaatagcctaaatgcctagtaataggaaaatggttacagcaacaaatgatggcatatctactcagtaaaatattctgcagttttaaaattataattatgaaaactatatagcaatatgggaaacgcttacaatgtcatgttaagtgggaaaaaatacagtactgcattaaatttaggttatggctgtagctatgccaaaataatgtatgtatctggatgagaaacagaaacaattttgaaaaatgaagataaagtgatttgctgggatatcaagattatgggtaaaaacttctctatttcagtgtctgctaaagttgttataactttatgcaattcaagggtcttggcaggctatgaaacaaggctgactgccccatctgaaactgaaagggtcacaatgcactgggtaagatgtgagcatgcaagagctatgtcctaaactgcggaagtgcacagacttctctctcaagggaaaaagccatgcatggcattccttctggcattctctagaatacctaggataaaactctgtgcaactccatgtactcaaatcctggggttagtgggtccatttagatgccttcacccatattcttcattgatattagcatcatgtgcccacaagaatcccagtaaaggtatcaacttcagagttcttcatcaccagggaattctctcactttcaaggtgttcccgagtaaggtgacctggctttgcagcttactagatgtatgatttaggtcaaggtacttaaccttgctaagcctcagctttctaaaatgtaaaatggatgtagtaataatgctgcctccatgtagatactgcacatgtgtaaataagacaagaaaagtaatatttactatagtagcttagtaggaagcactcaataaagggaagctattggtagtgtattattgcaaacgccctgatttttctaaatgtcaaggagaaacttgtgtttaatggaatagttttcaacttcatttgattatatctttgggtcttgttatagtgccttctctctcagtagctaattagtatcctctaagtttgagtcttttcaagcgttcacgcttccatggaagtgttttctgcactgtgtttaaatcacgttagagaacttaactaagcttcaagcctagtagtgtggtatgtgtagttattctgacattcagagaaatgcagaagttcaggccagatagaaggctaagagaaagagaccccttggtgatgccacagatgactaacccagatgggggagatgagaaggctggggagctaagagccacagccagacacaagagaggaatttggtttgaaaagagagtattaagacatcaacagcctggaaagagaagtacaggctgatttgtgacctgttgagcaccaagaaaaaagatgtggcaaaagaaggcacagatgttctgaaaaatgcaaagaatttgtgatggctcagagtccaactgagggaaatgactgccaaaggcagatctcagtggaccgtgggagcactggctcgatgagaagacatgaggaggctaggacttcataactgacctttaccagagtgtcttcctgacccagagcaaggaagagagatgaagaggagaagatgaacaagtcgtttgagaagaaaaataaattagcaagtctttagaagtagattaaaatattcctgaaccagcagcctgctttttactaaaattcttttttttaagtcatcttcaatggcttgaacttacagttcagttaagaggatgtgtgtagctctgtctacattctctcatcagaactcctactactcagagtcaaaagcttcacttctttccttctatccttatcatatggaagctgctacaataccttgagggtcatttcattcgtcagcctaaaggttactaaatgtagcttttatataaacaggcatccagttaacatagtggagattacaaagaccatacccagcaggaggtcagtggattttgatattctcctttttcccaaagggaagaggcttcccattggggttacaaagtagcaggtacttggctggaaggctgagaagagagaatggaggctgccctgttgaaagactgtgtatcagaaaggaatgtttaaatcctgaacttaggccctcacttctgcttgagcagagcagagctacttatgcctaagtattggcataagtgatgcctccacacagctttctcaaacccagatgtgagacaaagagagcagacagaaggcaagtgtccagggaaataaaatggcccacaagcagttatcctggcccagtgtccctgcaccaaaggagcttccccagctgtgtgtcacattaggtctcacattgcgtattctcaaggaggccaactaattctgtaaccatggataggtcctgtgataaattggaaaatgacttctggggcaacagtctctagttctcagaaaggaaattcacagaagtgagcggttggccagaaggccaaaaggccattgcctcagagtggatcccaaagcagagtgatgtggccattcacagcatgtttgagtatgtaagaaaaaagaaaaataagaaagggtatgatttctcctgccaatatcataagctgagaatatttttctttgctgaattcagtaaaataaatcagagatttttctcgatggataattacatttgttaaaaagcatttaaggaggtttgggaatctgggcatgatgttggtcccagatgaaataaagatgagaacatgatttagcttggagacaaactgtaacttaaaccacacctctctttttctttttccttattcatagttcatttcttcaattcagaaaaatagttttcatgggggaggggattgggaggagtgcaatggctaaaaaacaggctcagtgaaggccagaagattactaagaaacctcttcagttgaccttgagtttgctgcaatatccaaagatatatggcattctaaaatacaataatttaaggaaggcatttacagtttcatggtcatgtttaagcaataatatattaagaataaaagggaaagaaataatagcagcaaaatcctgcagaaagaaaagaaataatggtctgatgagtccagattttatctgaaattcctgaaataatgttatcacaccctaaatgaaccattcagcctcagtctgcatacttgcttggtattttgaaaaaaaataatataataccacaacatgcttgagcactcttcgtatagacttttaaattttactgaacttagcagtaatttattttaccaaaaaaagaggatggcatttgaaaaactatgtttaatctttatcttctgattttatattctgggcagatgttgggtaccaatggcatagatgatgtcctgtctggaagcccattcatactatcagagataacatgtagttacggctagacatttccaaggaaactatgcatctgtccacctgaaaataagtgaatatcacacagagaactcatgtagcattgacagacaccaaggaaattagtgaaagaaaacattggcctatgttaaacactaggaaataaagcaatcttgtcaacaatattaacagtcctcctggggccgaaagagtcatttgtttaccttatgaagatgcggctttgtgaagaataaaagtaaaaggccatgcccaaggattattagaaagactcttctcatactaattctcatttctgagtgcccacagaagtgagggccatctctctttccttaggactccggttttcaaaatgtagtttgattctatcacctgcttcaaatgactctctaaagggctctttgtgccctaccttgagctggcttctgcaatccatatggatgggcttgtctccttcccctaatcaagcccaactccaaattagacattttccttcatcagactcaacactgatccctgctttccttaaattcagcttatacatcttgtttacatccaaaattaaatctaaaaaagagaaaactcttggttttctcatcaaacttctcttcactactatactcccctattattagctagaaacattaatatgccatatatatatatacacattattgattacaaagtgggtcgttcatgaccatctggatatttagggtcatctttttagaaggggtgcttgatattatctagcacatttggttgtttacatgagaaactgaatctcagagaatttgggtgacttgtgcatccacacagaggatttggggctgtaatccaatgttgttttcaatataccatattgccttgtttttattccctctctgcatatggcactgtgctaggtgctatagaaacattccccaggacctctcaatccaagaacaacactgttgatacaccagcatgaaagggacatcacacacaacccaactggcactcaagtctgcaccatgcagttatttatatcatgtaacaccaaaccatctcctcccactcgccctccctccttccctctctcctgtgttcaatctgtcgctgactctggctgctcccttctctgcgacagtgccaagagctgtttctgctgctgtagaggctgtcttccaacagtttactccctgatgcaggcccggatgcctctctttaaggagcgtccacctcccaacactctccctccagacacgccagactgccctggcagccgccaccccttcacctgccatcctggccatgtcactcccacccctgggacaaggtccagatgtgccctgagcaaaaaacaggtccctgagtgaggagactaatggggtgcagggcccctgagaacagagggcaggtgccaagttacaaagaaaaaaagaaatgaaagactttagagagaaactaaaaataaagagaatgtgttttaatatccctttggctgattcatctttctgtggcaccagaaagagggcgttaccctcgactcacgaatgccttatgttttgttacaaggcctctcttcccacatctgaaagagtaaaataacaataagagtgtaagtacaggagataatgctggtggcatagctacaatctccaagctggagccagtctcagcttacagttgtgtcagtctttctaaaaaaaagaaaaaaaaatgagacagctaaggcccacgtatgcgattcctcaacatcccttcttattctttctctggcagttttcatcttattcaggcatgggtccaagatggagtagtgattaagcggcctggtgacactgtatactgggtaactgcttattcccaaagcatggctggcaggagagggagccctggttttcagtcattccagggaggaaatgaaatgctaatacctagcctaccgtaaactccacagtggcataagccttgctcaaaagtacatcagtgaataaaaagaatgtcaaaggctgtcagtagcagaagaggaaaggaaataggaaaagaagagaacagaatgagatgagacatgaagtagagagagatgagagaaatactatgctgggtgaaagtgattgggaaaccatttctgaccatgtatttcaccacactcatcccaggccttgcagcattgttttgaaataagaagcattgatattaataccaaaaagccaatgattttaaccagtggattatttgggacaaatcaaaggtaacaaggggtcaagataagcccatacgccttatttgacttgattgaaagattcattctcatagtgtctattctagattaaaactattcagaccagtctatacaacataaagtttggacattctttcaagagaacagcctttcatttactttagtgaataacaatatctagaaaggccatctgcttcgaccaaactttggaaaggatgcatttttaagattaatggaggaagaggatacctgcccaactagctatgttaaccaattcaacaccataataataattgtcaacatttatatagtttaccatataccagccattgtgcaaaacactttgcatacattctcacttaatcttcatgccagtcctgggaggtactcttatatatcctttttacatatgaggacctgaatcttagaaggttgagtcctttgccaaatgaaagcttaacagtaaagaaacagtagaatccagactgtctggttccaaaacttgtgcttttagccacttcacatattgcttctgtcattggtatgacagatgtcacaggcagatcatactcaaattctgtaatatgttctaaataatacaaacaatataactagaaaatggtagctaacttatatagcaggccccactgaaataattcaaacataaaaatgacctcttggcatttgattatctttcaccttagacgttgataaaccaaaccattcccttaggacaatatttttctgtccagcaaagcatttctcttgccaaagtataattttgttggctacatctatctcctccaagccctggaatcagacctaagctagtaaattgtgctgtcctggcaagacactcatcctccagggcattgtatcaggctaaagtaatacatcatgttgcttcccactctgtctgcccatttctccaaagagtcctaaaacagtaatgtttctgctttgaatctcttgctgttatcgcccctaggacttgcactttagcttcctggccttggaaaccaaggtgagctgaatagcattcttttcacacctgctcttgacttgggcttccaaaagaaaggaggctttcaacaaaggggcagccccatagaactctgcttcattccttttagactcagagaaacaaaaacattgacttgattgggtaacggaagactttggtcttttgggttatggggatttagtagctgagaaagagttgctgagtagtgagggtgggggcaggtcgtagagggccataattcctagaaaaagatgaccttgtccaattgagaggtagtgagaccagcaaacactggcaattaaaaattaacacaggccaaaagtgaaatcgatgcctaaatttgataggttttcaagaaaagtttatgacatgcagttcatagaaaagagaacattttctcagtgttcacttttaagatgccaaaggtcaatcttacttggaaagatccttccacagaaaagcagaaatgtacagggtctcctagcaaagtataaggcaggcaatagatatattagaaaatcttttttaaaaatcctaatttcaaaatgttttacagcagctttggtactacagtaattcacttaattccctaatatgtgtttgagtatatgtgtgtacatactcatgttattctaaacataaaaagatgtggccagttgacagtagtaaaagcttctttccatatctttactaagcacatgacaatgtataactcaataattgctttaattcaagttgtgatttcaatagacaaaaatttaaaggcaaaatattgactctcaaaaggttttcaagtaaatgcaaatcctatgaattctggaaatatcaaccattcaatgtattacattggggtcttggtaataatataatcacaattttccttatattagaaatttatgaaaggactcaagtcttccttataaagccatcagagatgtaggaaatattgggaggtgattctcccttttccacaaatgaaaatagtaattaaaatgctaaaatgaagactactttaagattattcatatcttatacaattttcctactagtatctcatgcaaaataatgcaaaccaaatgaatttatattaaataaggcggcccagggagaaaatcttgccctaaaaaactaaccgttccaagttattctaaattggcaatgtgtctggagttctgaaataaataactggtgtgaaaaacctaagtttccagattcttcagatcttaactgtaatatttggttgtgtaatcaatccttagttaatctaagtgaaatgcaggttcatttttattagctaataatttcatttgtgttttttatcagacatataattgaacattttctagaaatagcccttaaaatccaggttaattgtcttacctcttttttcctcctttcaaaaaaaaaaaaaaagggatcaactttggaacctcttcctgttagcaaatttctaaagaatagttttattccttagaactcaaagaataacaaatgtatatcttaagaactagttgcttcccaacttgaaaacgtttgcgtttttttattatttaaatactgactcttcaaatcaatgtcttccctttccaatgatagagttgaaatagttaaaataaaactttctcaagaagcaagatgggaagtttcaaaggaaattaaagctgaatgcattgttccaactgcattcccagaaatctataagaaacacaaacttatgcagcatcagtagaggaggtagtaccatttttgccagtgatgaacaaccagagtcctttgttctagaagtgatgaccccggttaaatctcagcattacacatttcccttcagagatggaggggagcgagttgcttgcatccattacaaaatgtgagactcagctaattggtgtggttttgatgatgccacagttctgcccaaaacccttgtaactagcaagtttgccctgaagattattgaattactccccgaccatgcattatttaaagggaagtgctgttattatagaagccagctgtttaaccattggaattctgtctgttggccaacttttaagatacagcagtacattgtgactaaacattatgtctagtcatggaggagtcaaagtctaggataggtggtggccaacttgtctggtaagcaatctttggtctacatgctatgaaacctttgaagcaaaactgacaaaataaatatataaatttttaatgtcaagactttgtcagcaagaaaaattatgtggaatgttatacatgcctattcattctaacagttgagtaagtgcatgaacacttcctcatgccagtacaacagaactgttggaccacaccttgctgtagaagggactataccaggcaccggtctttttttttttttttttttttttttttttcgtagcatcgagtaaggacaattaaaaagtgtttagagcatcttggggaacttcaaatagatagaaaacaacttttcaccttctaagttagaatatatttgcacctcttccttgaagtggtttccaaccatactgaatattgggcccaagccagttttctccttcatctcttcacttgttatagaatgtctggaatagaaaagaccatagcagtcatccactgtgttcaacataggaatcttctgctaggatctctgacagatgtttattacagcctttacttaactgtttcctgcaatggggagttcattacttttcattgaaatctgtcccatatgtaacacttgtaaattcaaattgttaggaatgcttttcttatattaagccaatatttgtttttcattcacctaccagctctgattctgcccactgggacagaattatacagcacctaatgaagagtttctcaaggcaaagataaaacaattagttccatctcatttaaaaattagtcgaaatgacaatagttataatttagaagtcattaaaaataactatctcaaataggaaaatgtcaaatactgaagccaattgttttattagctttctaataaattacattgtatcaggcctctacctgtagaccatagcagcaaatatctttattgtggttctggaaaaataaacaatatgtagcatttgaagatatggaacaaaactttgaccctcaactgtgatctttcaattggcgagaaatgagttttttgtttgttaaatattttgatgtagagccagaagaaatcaagcttacagggcacttcaaaagatgaattaaacttcaaaaggcaataggtaaacacttgttatattcatatattcccaatgacgtacaatgagtccattgtagacattatgagtagacaaagaaaaacatctctctaggattgtggtagctagagataattggtaattatatttatggcaaaatcgaagtctgtataccactatttcaacaatattttttaaaaaaacaaatgtagacataagataaactttctctatggaacattgcagaaaagtcctaaaattctctgtacctaaacccatcgatctacagactaaaaagcatgagctcagagtttagatgagcttagagtttgactgagtcatgtcaaattctgagctcatttaagcaaggtgataaaccagatgcctggtatttcctgttattttttttgctgtgatgatgatgactttctattttacaagaggaaatcgacatgtggtgagtttcatggttgtttacagtcaccaagaagtgaatatacaatcatagaaactctctctgccatcccaagatgaaggttaatgtcttcctcttatagcagacaaggagctccagcacctgtgggacctcagaagcagaattaagttagaggagcttgaaagggaaatactaaataggtcctggaatcccaaagcctgcccttgtctatcttcaaaggctaaccatgaattttgaaatcagttgtttctgagttggctaactttgaaagtttctggagctcttcctaccagcccacccccaaacatcaagttaccatttcataatcaacaaatgaatcccaattacatcccattaggaaatttatagtttcaacaaagggcaatcattacctgtaatgggcaaaagatacaatgatcacaaaataggccaagtcaccttttccttcctccccacccacaagagagaagccaaagggtatcagctgtgctttactggctagccacttagcaaaaagtacgtagaagtcctttgtggattacaaaggacagtgtaagcccatttgtgtctttcctctaatttatagttccaggaaaagattataaaaacaaaaaaacaaaaaaatattgtttacattgctcaggggacccgctgagacatggcttgccaggtcccagtccctcccaaatgccacagccattgggagtccacagggtctgcttcgagatctgcacagcagactctgcccagaaagaccacaaatgaaccctcactgagttactacctccagctgccctaagaatgacaataaacctgccttacagaaggaagcagccaccagggtcctgtgccaagcatcaggaaagcatttgggttcttagaggttgccatgaatgctggcccttccagcagggaggcagccaaactggtttggtagcaggccattaccatgggcctgatctcccccaatgagggagtgcaaaatacatgaggattctgactttccaaaccctatcctcaagtaatggtgtcattattacagaaacatagacttgtgatttaaaacgtaatgttttcttctgtcaggaattaagattgaatacatgtcagattaataaattggattcttctgtgcccgtgatttataaaatttcttccataaagcctgagcatccgtggagaagaccaggagagataagggaggagaagagaaagcaagtttctgtccctctgcccttgcctgagctagaacagaatcacatgaaagatggacatttcgttcactgtttcagtttggcagttactcaataagaaatagaattcattgttctggcctcttggggtggtaagagagggagccctactagattggtattgtaattagtttcaagtcccaagacttctgtacacggtcaatagtgacaatgcttcttcacaaaccacataggatcctggaaacggtactcgcagaaagctggctatgatttacttacgacttacactatatgtcaggtgctgtgctatgcattattcatacttgatttcatttaatcctcaaacaattcttacaaggaagatattattaactccattacataaatgggaaaactgaggcccagagagcttagtgacttttccagagtcatacaacagttctcctgggctcctcaatgaggcacttgggctttgcagcaagtagatctgagtttgcatcttgactccacgacttactgcatgaatgaccttcagcaagtctttgccacctctgagtctcagttttcacattagtaaaatgaggagccctttccaaacttaaaaactctttgatgttcaacctgactatatagtcaaagcagttaaacaaaaaggcaaacatgtgagctgtcctgcttataaaagcaatgcagcgttgccaacaagattaagaaaacaatgcatgatcttattttaatatttctatcatcctctgaagccaatatataagaaaagactaggattgggcatagacaagatccttgcctacaggttgcaaaacttggggagagttaccacaaatagcatggtgaatgagttagtgctaatgagtgtggtacagactccgggagacatactggcattcagaaagatacgtggggtcaggaatcattggggaagtttcaagaagaggttgagacattgcagaatatgtaggttgactagctgtggggagaggcaggagctgggtggaagactgaagaaagcagattgcaccctaacatgaggccactctgtttgatttgtgcagacaagcccacagggtatggctccagcagtcggagggcgcctcagacaggcatcgtggatgagtgctgcttccggagctgtgatctaaggaggctggagatgtattgcgcacccctcaagcctgccaagtcagctcgctctgtccgtgcccagcgccacaccgacatgcccaagacccagaaggtaagcccacctgggtgggatccagccatcctcaagtggtctctctcttgtgcatgtgggtgggccaagcagaaatcctgccccatagtctcctggcttacaagtcagaaaagctcctttgcaccaaagggatggattacatccccatctctttggtcactctgcattgcaaatttcccctcccaccgctatggacgatgtgatgattggaagatgttacaaaacagtggctaaacaaacatgggctttggtgtcagacaaaagtgaagtcctggctttctcacacaccagcttagagcccttggcaaataatgtgatgtacccaagcctcagtttcatcagtaacattgggataataataatatctaccacatcagtttgttgtcaaaattaagtagctcatgcatatactttgagatgcttttcacatgcctgcataaagtaattgttggaccatcgttaatgtctgccataattgcacttaataacaaagcttgtaacctttcaagttctgagattctacaatcttccaaagaaaataaaaggctaatgggaactattcaaaattcatattcagtagcaagcataattaaacatgaaacattaaaaatagaaatttctgtttggctataagaatgcctagacatttgtaatgatcaaaatctgcaggcatcattttctaagagctagactgtaaacaaacctcagaggtaccaactatgccatcagtagtacataaaacatctgatgcacatttagtcacttgatcgatttctcttgaatgagtgaacgaatgaacaaatgaatataagagattaaaattttagccattaagtagaaagaataagaactaaagagaaggtaaaggaggaaaaagagaaggcaaggaagttgagtaagggaagaaatagctctcgtttaagtattttggggactctgttgaaaaaagaaatgccaacatgtggttttaatctttggagctagaactaataatattgtgcaaaagcacaagatgagagatcaagaagttcaccatgacaccttcgctgcttcctggtcttaaacctcagctgaggctggaagaggaccatggtggcttattggagatgtgaccccagggagcccctctgaaggatggaaggggactgggcaagacccaacacacacagaacacagtagccactggccaggcaggaagcaaggatctcagaaaagacttttaggtgaatgtggcaggaaagcgtgcttgctggggcaaaggcagattcattctttctcttcccaggtgacccagcgcctcttggtttctaactggggagggggtaggtgtcaagagatgagtcccaaagttctggaatggtgggtcttgtgactgaggtctagacccctctccagcatgagtgctgtctcctgcatcatatggagcctgggcattctgagctcattcaaagggacaccatgggaaccacttgttctcaatgcaattatttttgtgatgtttacagtatcagcccccatctaccaacaagaacacgaagtctcagagaaggaaaggttggccaaagacacatccaggaggggaacagaaggaggggacagaagcaagtctgcagatcagaggaaagaagaaagagcagaggagggagattggaagtagaaatgctgaatgcagaggcaaaaaaggaaaatgaaggacaggaggattaaacagacagaggcaaggatgatgagagaggagcagacagcaagaatgaaaagcagaaaatacaatagaggaaatgaagaaaagtaggcctgctggagctagatgatgatgtgatggaaatagaagtaaccttttagagaatctcgctaagaaacatggagaaaacggaaaagaaaaatgtaatgccctagaaagcgcaaagaaagacagtggcaaaaatgaaaaaaaaaaataaaaattataaaagaggcaaaaaaagacacactattctctgcctctaaaacacaattaaataaaagaatttaaataaaaattaaggcttctatatgcatttttaaattttgtatgaatctgttatggaagaattgcctatgtcaatatatgttcagagttaaatattagccccaaatgctcagcaagactgaattgtgtcatagaagttcccagattcccttttcccgcaatgtcattggaggctgcatttcttagtcaagtccagggtttaggccaaagggcatccggtattgcctaaaaccctgtgaggtctgtgaggtaacttttgagaagaggtcactgcactcttcatcttttttgcactttggaatcagatataaaagatgtataagtttgctagggctgccataacaaagtatcataggctaggtagtttaaaccacagaaattgattttttcatagttctgggagttgaaagtccaaaatcaaagtatcagcccttgcaagggccttagagaaggctctgtcatgggctcctcccctcggcttgtaggtggcctccttcttctccccctgtgtcttcacttcatcttccctccatacatatctctgtgtctaaacatcctctgtgtgaaacaacaccagccaggttggatttgggcccaccccactgacctcattttaacttaattatctctgtaaagactctgtctccaaatacagtcatattttgacgtactgggagttagggcttcaacacatgaatttggacacaattcagccagtgacagaagacttctgatctctgatgataaccactgcattttgattacagctcctagaaaacactcccctccaccaccccaccacagatctatttttatatctgaaaccctgagtttctgctccatgagaaccccaggaacatactatgttagatctggaagaagcctcagaaatccccttattttgaagactaggacactgagatccagaagtgggtaaagatgtgcttgggttctaagctgctcttcttttggccaggagacaacagcacataatcaaagtgggtcaactaagaaagaattccagaaggaaaagagagggcagaaatgaagggagagaatgagagcaaaagtgctggatttccctgagggtgaagaaaagttaaatagaatcacagaattcagattttagagatcttctccttcagatcccttggtttaatcagtaggattggggtcttcatagataataaagcaaaaactctcgccatcctccaagttgtgaattagaagagctgagaaagggtacaagacggaagttctctaccaaacaaatggtgacattttggggtaagaatatgactaacccagaagtgaagcatttcatccaagtagtctattttgaagatgtcatggtataaaggaacctcctttctgcctggtcctccatgcctctgccatgctttttactccaggatcaccctttctagtggttcactgaaaacccaggattacttaaatatgatggacatgttcacggctcaatccaggaggaaaaggtcgaactgaaagcatgccaaagccccacatgggagccaagccactgctgctgtggttgcaaagtggatcctggcttatcagagcagagagaagccaggctcgtgccttagcccaagtggccagtcaccttattcaggagatactaagttctccagctaagacatccatgctttgggaccagctgcagacagaagccaattcctactacaaccatcaccttagagtagcatatagacacagatggctcttcaaaggaccacagttccatggaataactaagaattcatgtcctgtggaaaggtttgaataaactataattatacccaatcataaatttcattcaagaagaactaaagcaaaggcaaagacagagagaagaaggaaggaaggagggagggagggagggaaggaaggaaggaaggaaggaaggaaggaaggaaggaaggaaggaaggaaggaaagggaaggaagaacaaaaagactttctagttaaagaatgcttaactagcaaactatgtactataagacagttcttttcggaatgagttttatcaactctaaagcaattatcttgaatgcctacatgtgattactgaataatatgaaccaagaaaacagaaagaatctatattatctttccatttccttctttccagtatcaatacccaagcctctagtgatacatggcatataatgttggatggatggatggatggatggatggatggatggatggatggatggatgaatggatggttggatggacaaatgagtaacataggctgatgaatagtggtagaaagacacaccataaaaacaagtggcacttctgagatgaaatgattcctattctcctacacaagacagtgaggcaagtacagagtaaaaaaggaaaggcataggagctatgcttatacaagtattgtatgtttggaatttccttcgctggccaaattgaaattgttcaaggacctattgctacaggtggcaactggctaagaatttcatagtgaatattatacacctattactccccttaatgtttctttgaagtaagcagaatattaataatcatttaaaattccagtgtttcaacttcaattgtttcctagggcaaattgataattgtgtgtaaaactaattggaatatgtatggaataatcatcctgaaataaaattggtgaaaagtatttgttattgggcatctacaatgtgcaaacctctgtactaggcatgaacaagagttataagcattggagaggctaaaatatagtccttaaggctgggcacagtggctcatgcctgtaatcctagcactttgggaggccaaggcgggcagattgcctgagctcaggagttcaagaccagcctgggcaacatagcgaaaccccatctctactaaaaatacaaaaaaattacctgggcatggtggcacgcacctgtaatcccagctactcaggaggctgaggcatgagaattccttgaacctgggaggcagaggttgcagcgagccgagatcctgccgctgcatcccagcttgggtgacagagtgagactctgtctcaaaaaaaaattaaataataaataaatagtaaaatacagtcattaagagtacaaaatgtagattcagactacctgggttcaaatcttggctcttacttgcattgtggctttgggcagatcatgtaacttatgtgtgcctcagtttcctcatctgttaaataggggcaacaactgaatctaccttattcagttgttgtgagggtttattgagattgtgtgtgtgtatgtgtgtgagtgtagtgtgtgcatgtgtgtgtctgtgcaaggagtgggaggtgtatattcagagacacatattacagcacttaaaatggtatctagcacttagtaagcattattcaagttttagttaacattattttacttacctctgaaaattggagctatgtgaaaaagaagttggtctcctgaagtagaagccagtcttgtgtcaccaaaaacttcaagcccaagcttgccaacgcttttccatgatgtggtagtagagtttcaagcatgtggtaggataagagaactcaatgacctaagaaccattccaacccagagaacccctggttctatgaataattccaacttaaataggtagcttggctctcccaagtgagagccattgcttctgtttccgggtcatataatgaactttcagaaaaccaccatttttctcaaccagttaaaattaagtgtaatacgtgctttcatttcatggtgcctggggaaaatttaattgtagtatgaactccagttattggtagtcttaagtaaaattgccaaaataaatagaaatgcaggatatttctgggctcacacagcttccgggacactttagtttcttgggctgccaatccagtgcctttcacaagcatttgatcttttttcaaacatctcttgaaaacaaacaaaacctcacacagcttctaatgtgtgcactgttcgaatgtaagggtggaaaaggaggcaaagaaatgagctcccaaagagcaattccccttctctcgcctccatcccttgacgacctccctcccactaaagggaaacattgttttcttaggtaataaattctgcaatttctcaagtccattaacatccactgggcaagatgagatctattctttttatttgcccataggaaaagaatagtgcttttttgcaatattcactagataacacagagttgacttttaatccaagggcaacattgatagtctctagttaaaggggaagccttcaggagcaatgaaaagattaatagttttagatgaagcagaatccaaatccctttttatgagttttgaaatatccagtttgtatgctcacctcaatacttaaagcccagttactgattcctttggcctaagcaagacaggtcaatttttaaagagggagtagctgaggttagcaaaaattctccaggtccacaaaacttccagacctgcaaggtgaaaatcagcttttctgtcatccctaaaggcctaactggaatcagaacttttccctgatgcccacatatttggaggtccttttttaatgggactccttaatgcctttagtgccatcccattttcatccagtgtccaaaagaaatgatttaaaaatataaacgtatgtttaaattccagaagagagaaatggagattgagaacaatagggaaatgatgagagctatgggaaaagaggtttatgagtccatgtctgattcttccagagagcccctaagaaagttcttatcataccaggaactcaattataactttcattgcctattgttagatgagtaacaggagctagaaaacattttggaaattcccatctttatttttttaactaatatgattatagttttaagaaccattggtcaagaagctaactttttaaaaagtggaagtatgatggttagaaataagaatgctaaaggtgcatcaagctgattttaattctaaatgtccttggcagcaatttagaatctgtaataaactacaccaaacagttttgaggggaaggggattagtttctccccttccttcgtgtgtgtgtgtgcgcgtgtgtgtgtgtgcacctttgtgttctagcattgttgcacccattacagagctggggggaactattttccaaaattataggtgagaacagtttcttggattgtctttcagtgaaggtaaattcctctgtaaaaactaaccatcattcagtaaaaactgcaggattcctttgtcttctcaaaagcctgtttctcatcctaaattaaaaattattcaggaaatagagaggacattattggaggggtggaaataagttggttttctttttattgtatcttttgaggatccagggacttctaccatttcccatctaacatacagagaaggattctctaggtccctgtctatagactgcagtaactttcctatagaaccaatttgcaattttagaaatttctaggtctaattattgacccattacaaccaaaggtcaatgcatccagccaatcttccttctatcatcccctgcccttacttctattagggactgggattacaggcaaaacccatcaaatgcctcttctaccactttcccatttcttaaccattagcctctaacttcctctattcagtttctcatatgctttcatgcccattgggtcagataaaggaacattcatttatttgagtaggcatctgttatgatcactccggaaaaaagatgacaatgggttaccttgtcctcctgggcttctctaactgacatggtcaaaatgcccatatgaagataagatgttaagagcaagatttatgaaaagctgagtatgatggcagctcttgtctcataaaataactcgaaagttcccagtgaaagaccaagaaattttacatcaaacccaaaccggccaaatggtccaagcttccaagctgggatccatggctaaagtttctacaaaattctgggtacaatgtataaacattcacttggggctttctgtctagccagcaccaagaggtcaagtaatcaaggaccaactagccctgccatctgtgaaaatatgtgctattttcacggctttagttcacaattatggcaagacaaaagttccaaataattaggagcaagaccatggcaggttgacggttgagtaaggttctcaatcagccgacaattgtagagttggggatgtgcaatgtttatgtcatggtgtaagtatgtggcatgcttgactagcttgtgaggcactggaagactagaaggaatgaaaaatatgaatgaatcaataaatgcatagtataattactgttattttgtcagtattgttttacctaggtcactattgaatgctctgatttgtctctttataaataataatatgttttcttcttcaaaagaacactaggatgaaggtagaggtgcttttggcacaatgccacaattctgatttttttaaaactgtatgcatgcataaaatgttcttgagccattctctgccttggaatagcactggctggcattctgcatgtttacttttatatgctgaaggcccccatcaacctcaaacagaggcaaatcaatttaacttctcatagtgttattttgttcatcctaaaagttcaagagagccttccaaacttccaaaatttctctcaattcagtgaggaggaaaattcagaacacagcatttgaatgttctgcccagatttgtcacacacacaaggaatgagtgaaagagggcaacaccctttcctcctaaccctgtgaactcatcactattgcattgaaatgacaccaaaaggtaaaaaccctaggcctcacatctcccaagaacactgcaataggagttactgcatacaccagtttaagtaactctagcataaattgtatgtcagatgaaacaatggcattttggaggcttaagagaaaaagaataatcaaatccagtttttaggtactaatgtgctgaatctttagcacatagcagcaaaattgctagaatctggtgtttcactttttaaaataccacatttgaacctttcagcaattccaaaatcaactccctctgcgaaagataataagcttaaacattttttaaatttaaaaatgtaacacaaacaaacagctaagcaaacaagctgcccataaaatcaacagtctggggagccctgatcctgaagtattttacaacatccttcatgactattaaaggcaacataaacacctcttgtcagcaagggaaactacccttggcatttttttttctttgttccccaggcttttaaaccattttgatagagattttttacatcacaggcagaaatatttgaaatagagtcaggtggtagtctttaaaagagtaagaaagttgctaagtcaagataatcttggaataaagtcctctgattcctggggattcctagggatgccccagtcactagaaaacagagctgtaagtccactctcccagcactcaacggagctccggaaaccaaggagctagctactgtttccccacattcagccagagaaagggcagcactctagcatgcaaactgctttgacaatagtaacaattaaaaagtaaattaaaaagaatcataatagctgatattgattaggtacttgccctgtggcaagagctatagggaatcacctcatttaatcttcacatgaagcttgcagagtgagtaccacaattatcactattgtatagacaggaaaactcaggctgagtatggctaagtgtcttgccaacgtcttgggctaacaagcggtcaagcagaatccaaacccgagatagatagaccacagtgtgctaatcaagcactgcactctctcctgcatttcttagttgatatttaccatatacaatctgtcacttgtatgagatggcagggggttctgtgctatttgtccttgtagagaataccacaggaagaaagtaagcagccatgcaatatttgctgttgacctgaactccattccatcattcctgcaggaaattcgcatccattaaatgagcatttcctggtttgccactttgctcaaacactttgcttggatctggagaggatatagaagtgaaggaaatatgctacctgctctcaaggaacttatgttttagtggagagacaaacatgcagaatttactctacagaacatcaatgcttgagcaaatgtagacccagagagggctcttacagcacacaagccagaacagactgatggtgctaacaattaggttcaaggtttttctaaacagtagactctcctgcatacaactataccgcatgccaggtaaatgactgagggttattacatccaattataacaccactgtgatgtaggtgctcttaccccacactttcattttacagaagaggaaattgaggacagcacaatgtagtgattatcaaaggtcacacgactactgtgtgggagagctaggatttaaaccagatgcataagatgaggtcctccaagaaacagaagatgagaaggtgttaaatgagcaggggttttattagggggaattaatgtgtgaacagaaataggggaggataggcaaagccatcagattgcaaggcaagcctaaccccaagggaaggagagagagagagtagattggttggaaacatttttggtgggtctatggtctaaggaaagttcagcaaagtcatcatggagtttttgagccaaagttgggcaatacagttgcccaacaaatttctgtgtttctcagaaataggtctgcctcaatgtccccaccatacttggtcactggctcttgggaggggcctgccctgttccaatccactagagccaaagaagagccgttgtactggcagggggtgggggaattcctacaaccacataaaaagtggggtgaggtttccagaaaaaaacgtgatgctgggctaaccaaaactgtgtccagtaagtacatatccctcactctgttaaagaagcagccacataaacaaggagtacacgtttctcaaaatgtgcaccttgttctttggttttgaagtcacatcccaaagtgctgagtagatcgcatgaccctcgctttgcctggctgccagagaggaaaggctgatccaactctcctggaatttgaacttgtgattccctgaagtaaagagatatcaaagttgatactgagacatctaaatcatcctccaccatttcacatgtccccaggccaagccagcaaaattgctatagcacatccctttcaacaggtaaagggctgatatctgagccctctttccaatcatccactgctcttttcttctcattttgccctttttgggagcaggtcaatgctgagttagtactttatgctgtacaataagctgctgatattccatgctggacagaattttcccagtattttttatagagtgccaggcttttcctagacttcatgtcatacaatacttaacttgtttggagtgggtggagatggaaacatagtctattgaaaacatcactgcttcctccctgaagtttaaagagcctatttttatccttttagattctatctctcaggcaaaatctcataaagataagtggggaggaaaaaaagggggttataatacctagggagtttgcttttgctaattgaatactgtgctcctagacttctataaataccattacaaatgggtcccagcttgtggtaatactcaccctcctcattgagtcttctgtcccatggcacagcctttccctccaaactagcatctacccccatctggaagcatgggcagctcatgatattatcaactattgctattggaaagtgatttggacttgaaagcactagatattttttacctcttggggaggcagtttagcagagtggttaactggtgagctccagaatcagaaggaataggtccaaattccaaccactattacatctccatcataagaaattaggcaagttgtttatcctaagtttcagattccttaaagataaaacagtcaagacagtagtacttatccctgagagaagtataggaaacaagaaaatatatgcaatttacatacatactacaatccccagcacatgacaaatgttcaagtaatgggaactgttattattttagccctttgtctatcagtttgttcctctgtgacctcaagcacattactaaatgttagcgagcttcagcttgtacgtgggactgacaggaataacaccgcatcacctcatgtggtgattgtaaggattcagtgatattattttgtaaactgtaaagcctttgcaaatgttaagcaagattattattattgccgttgttattagtcctcagtgatcttttttttttttttttttttttttttttttttttttttttggagacagagttttactctgtcgccaaggctggaatgcagtggcacaatctcagctcactgcaacctccgcctcctgggttcaagcaattttcctgcctcagcctcctgagtagctgaaactacaggcacacgccaccacaccaggctaattttttgtatttttagtagagacggggtttcaccatgttggccaggctggtctccagctcctgacctcaagtgatctgcccacctcggcctcccaaagtgctgggattacaggtgtgagccaccacacctggcacagtaatcttaattgaaaagtctgtggatagctttccaaaggaaagcttggagcttggataagaaccaagagataatgggagaaggtgaatggcctcttcagggccttttctagcaccctaaatatgcgtgtctgtccataatgggtaatcatatatatcacaaatcaaaccctccacaaacttatttcctaatgtgtttgttaacctttccttctaaagggtaaacttctttaaccaaccccagtgagctggaggatcaatgttttcttaatagtcttaccttcgttggtgtcaataggaaacagtatttactcactactgttttccttttaaaaatctgtctagttgcatactagaaacagtttcagctggtttgtttgtattggacaagctgctgaagtgaaaagtttttgcttgactgaatgtgagacagtttcataactcttcaagaagtgcaccaaaggtgggtgccagctctgatgacggctgcttctaacatgcctccacttgccgcccattgtcaagggtggctggcgtaattaagttaagacaatgagcaaagcaacagatgcaactgagacctagtccctgagtgcttttgttttgtcactgtcattgtctgcaacaaagaagtcacatgtgacagcctgggaagagagccaaatgcaaaccagacgatatcccagctggtttgaatggcctccaccgtgcacgtgtgtgcatgggaatcatgctacttggtacagcatctgcttcactcaagtgagtttcagcccatggctttgctgtgatgctgagacagacccagaagaaacagaccagggaatccctccgctcagactttacactttataccttgtgctttgagagaaaagaaaaagaatctctctattggagacaaaaaataggatgtatgtggttggtcaatctaacctcaattctttttgctatagccccccgctaatttaaagagtgaagcatagatggtatcttaatgttttcttgtagaaatttgggattaatttggcttgagaggaagaatggagattaaacgctttatgaggctttcttttaatttgttcccatttcattcctgaatattttcttagtttgggcattgcagatgtttaaagaacttcttattttgagctggtatgcctcttaaacagaaaaacaaaaggtaaaattcaaattagtgtgtttctccgcctgttaattaatttggttagtagttaggcagagagatggcatccttaataatatctattttgcgggtttgatcagctacagaccatcaacagtgttgattgagaattgaacaaaaacatttcaaggagtttgggaacattagggatgctattctgtggccccatgtgtccttctctcatttttctagagaactcctataagaaagcagaacacggccaggcatgatggctcatgcctgtaatcccagcacttcaggaggctgaggcaggcagatcacctgaggtcaggagttcaagaccagcctggccaacatggtgaaaccctatctctattaaaaatacaaaaaattagctgggcatgatggcgcgtgcctgtaatcccagctacttgggaggctgaggcaggagaatcacttgaactgggaggcaaaggttgcagtgagcctagatcacaccactgcactccagcctgggtgacagagtgagactccaactcaaaaaaaagaaagaaagaaagaaagaaagcagaacccaatggaagattaagaacacacatttagcttacgcctgtaataccagcactttgggaggccaaggcgggtggatcacaaggtcagaagttcgagaccaacctggccaatatggtgaaaccccatctctactaaaaagtacaaaaattagccatgcatggtggcaggcgtctgtaatcccagctactacagaggctgaggcaggagaatcacttgaacccgggaggcagaggttgcagtgagctgagaacgcgccactgcactccagcctgggtgacagagcgagactccatctcaaaaaaaaaaaacaacaaaaaaaaacaaaacacaagtttactgggaacttagcagtagatgctttgcaccacaacaaatgtatcttaagtggtcttttgtgatatttgagggaaagtgccagaatttaaaacaaatggcatttcaagttattctatacaaatgcccagtttctttctaccatctttttttcctttttgcagtggtcactgagctattttagtgaatgtttttacacaatgatgccatcttccttctactcagtcagtacaagatgttgaccatcgactcataaaacactagctacctttcatgaaggacttggtgataactctcatgttccaagtagaaccggaaaacatgtgtaagaaaacctgccgatccctatgggccttggccaataggtattattcccaaggggtggcagtttatctttttccccagccttcatattaaaacctctcaccttctccaggtctcaggtctgtgtaatctcaaatgtgctttagctcctcacaatattgtaactgtgtgggtgttcattaccttagccagaagacagtttacagattccaggtctcatggagagaacttttgtttttggttatgaacctcactgtataccaataattatccattacatccttctgtagagggctctctggctagagataaaaccaaaaaaagaagtacctcaggtttatgcatataaatgccagttcctccttgattttatttcaaaactcctgtctacatactttgcaatttaaatacattcaaggataaagtaataactgtaggaaaagtattataatataatgacttagttctgcacatcacaagggggtccctcatactcattcattcatttcactcattttacagatatttattgagcacctgcaataacctgcacactgctctagacactgggactataacagtaaacagacagatacatctctggtctcacagggcttctattctaagcaaaactcaatatccaggccgggtgcagtggctcatgcctggaatgccagcactttgggagaccaaggccaggcagatcacctgagcccactagttgaagaccagcctgggcaatatagcaaaaccccgtctctacaaaaaaaaaaaaaaaaaaaaaaaaaaaattgtcaaggcatggtggcatgcgcctgtggtcccagctacttaggaggctgaggcaggaggattgtgtaagcctgggaggcagaggttgcagtgacctgagatggcaccaccacactccagcctgggcaacagagtgagaccctgtccaaaaaaaaaaaaccctcactatccttaagataacatcattgcttgttgatgagtgaatgttaacaccaaattaggaacccaggacttttagtcttggcatggttactttccaataaagatgacaatactaagaagagaaaaatgatttaataatgataatagtggctaatacttatgtagtgcttaccatgtgccaggtctattgtaagtacttttatatatattaattatttaatctttgatcctataaggtagatattattgttaccctagtttatagatgaagaaacggaaacacaagagattgccactcatacaagtttacacagccagaaaatagaaaagctacgagttgagctcagcccagtatgtctatgattttacagactcaaaattaattataagatttcctaatcttcgatttctgaaactctgccttgctctagaggaaaacaagaaaaacaatgaaaaataaatgtctcttttttacaaaaattaaaacagaacaaactgcaataaaacaacagaggatgaatccagaatgtgattgattttttttcttactaggaaaggatctagaggccagaaggctggatttttcaggatctcctttcaatcaatgaatctgtgatagaagcagatgaatcaaatctcatctttgtgtgattataaagctgtctgtggtattcacgccaccaggggtacatagaagatgcctgagtgaggtttggcaaaagtactaagggcctgtccacctatacatgcccttctcaggaaaaccaaggttcaagctctctattagctcaactggtaaggcgtaagacatggaaggttgaggcccaatgttagaaatagatggatacataaaacttcatcaagttaatgtcactttttctcctttatttataggaagtacatttgaagaacgcaagtagagggagtgcaggaaacaagaactacaggatgtaggaagaccctcctgaggagtgaagagtgacatgccaccgcaggatcctttgctctgcacgagttacctgttaaactttggaacacctaccaaaaaataagtttgataacatttaaaagatgggcgtttcccccaatgaaatacacaagtaaacattccaacattgtctttaggagtgatttgcaccttgcaaaaatggtcctggagttggtagattgctgttgatcttttatcaataatgttctatagaaaagaaaaaaaaaatatatatatatatatatcttagtccctgcctctcaagagccacaaatgcatgggtgttgtatagatccagttgcactaaattcctctctgaatcttggctgctggagccattcattcagcaaccttgtctaagtggtttatgaattgtttccttatttgcacttctttctacacaactcgggctgtttgttttacagtgtctgataatcttgttagtctatacccaccacctcccttcataacctttatatttgccgaatttggcctcctcaaaagcagcagcaagtcgtcaagaagcacaccaattctaacccacaagattccatctgtggcatttgtaccaaatataagttggatgcattttattttagacacaaagctttatttttccacatcatgcttacaaaaaagaataatgcaaatagttgcaactttgaggccaatcatttttaggcatatgttttaaacatagaaagtttcttcaactcaaaagagttccttcaaatgatgagttaatgtgcaacctaattagtaactttcctctttttattttttccatatagagcactatgtaaatttagcatatcaattatacaggatatatcaaacagtatgtaaaactctgttttttagtataatggtgctattttgtagtttgttatatgaaagagtctggccaaaacggtaatacgtgaaagcaaaacaataggggaagcctggagccaaagatgacacaaggggaagggtactgaaaacaccatccatttgggaaagaaggcaaagtccccccagttatgccttccaagaggaacttcagacacaaaagtccactgatgcaaattggactggcgagtccagagaggaaactgtggaatggaaaaagcagaaggctaggaattttagcagtcctggtttctttttctcatggaagaaatgaacatctgccagctgtgtcatggactcaccactgtgtgaccttgggcaagtcacttcacctctctgtgcctcagtttcctcatctgcaaaatgggggcaatatgtcatctacctacctcaaaggggtggtataaggtttaaaaagataaagattcagattttttttaccctgggttgctgtaagggtgcaacatcagggcgcttgagttgctgagatgcaaggaattctataaataacccattcatagcatagctagagattggtgaattgaatgctcctgacatctcagttcttgtcagtgaagctatccaaataactggccaactagttgttaaaagctaacagctcaatctcttaaaacacttttcaaaatatgtgggaagcatttgattttcaatttgattttgaattctgcatttggttttatgaatacaaagataagtgaaaagagagaaaggaaaagaaaaaggagaaaaacaaagagatttctaccagtgaaaggggaattaattactctttgttagcactcactgactcttctatgcagttactacatatctagtaaaacctcgtttaatactataaataatattctattcattttgaaaaacacaatgattccttcttttctaggcaatataaggaaagtgatccaaaatttgaaatattaaaataatatctaataaaaagtcacaaagttatcttctttaacaaactttactcttattcttagctgtatatacatttttttaaaagtttgttaaaatatgcttgactagagtttccagttgaaaggcaaaaacttccatcacaacaagaaatttcccatgcctgctcagaagggtagcccctagctctctgtgaatgtgttttatccattcaactgaaaattggtatcaagaaagtccactggttagtgtactagtccatcatagcctagaaaatgatccctatctgcagatcaagattttctcattagaacaatgaattatccagcattcagatctttctagtcaccttagaactttttggttaaaagtacccaggcttgattatttcatgcaaattctatattttacattcttggaaagtctatatgaaaaacaaaaataacatcttcagtttttctcccactgggtcacctcaaggatcagaggccaggaaaaaaaaaaaaaagactccctggatctctgaatatatgcaaaaagaaggccccatttagtggagccagcaatcctgttcagtcaacaagtattttaactctcagtccaacattatttgaattgagcacctcaagcatgcttagcaatgttctaatcactatggacagatgtaaaagaaactatacatcatttttgccctctgcctgttttccagacatacaggttctgtggaataagatactggactcctcttcccaagatggcacttctttttatttcttgtccccagtgtgtaccttttaaaattattccctctcaacaaaactttataggcagtcttctgcagacttaacgtgttttctgtcatagttagatgtgataattctaagagtgtctatgacttatttccttcacttaattctatccacagtcaaaaatcccccaaggaggaaagctgaaagatgcactgccatattatctttcttaactttttccaacacataatcctctccaactggattataaataaattgaaaataactcattataccaattcactattttattttttaatgaattaaaactagaaaacaaattgatgcaaaccctggaagtcagttgattactatatactacagcagaatgactcagatttcatagaaaggagcaaccaaaatgtcacaacccaaaactttacaagctttgcttcagaattagattgctttataattcttgaatgaggcaatttcaagatatttgtaaaagaacagtaaacattggtaagaatgagctttcaactcataggcttatttccaatttaattgaccatactggatacttaggtcaaatttctgttctctcttccccaaataatattaaagtattatttgaactttttaagatgaggcagttcccctgaaaaagttaatgcagctctccatcagaatccactcttctagggatatgaaaatctcttaacacccaccctacatacacagacacacacacacacacacacacacacacacacacacacattcaccctaaggatccaatggaatactgaaaagaaatcacttccttgaaaattttattaaaaaacaaacaaacaaacaaaaagcctgtccacccttgagaatccttcctctccttggaacgtcaatgtttgtgtagatgaaaccatctcatgctctgtggctccagggtttctgttactattttatgcacttgggagaaggcttagaataaaagatgtagcacattttgctttcccatttattgtttggccagctatgccaatgtggtgctattgtttctttaagaaagtacttgactaaaaaaaaaagaaaaaaagaaaaaaaagaaagcatagacatatttttttaaagtataaaaacaacaattctatagatagatggcttaataaaatagcattaggtctatctagccaccaccacctttcaactttttatcactcacaagtagtgtactgttcaccaaattgtgaatttgggggtgcaggggcaggagttggaaattttttaaagttagaaggctccattgttttgttggctctcaaacttagcaaaattagcaatatattatccaatcttctgaacttgatcaagagcatggagaataaacgcgggaaaaaagatcttataggcaaatagaagaatttaaaagataagtaagttccttattgatttttgtgcactctgctctaaaacagatattcagcaagtggagaaaataagaacaaagagaaaaaatacatagatttacctgcaaaaaatagcttctgccaaatcccccttgggtattctttggcatttactggtttatagaagacattctcccttcacccagacatctcaaagagcagtagctctcatgaaaagcaatcactgatctcatttgggaaatgttggaaagtatttccttatgagatgggggttatctactgataaagaaagaatttatgagaaattgttgaaagagatggctaacaatctgtgaagattttttgtttcttgtttttgttttttttttttttttactttatacagtctttatgaatttcttaatgttcaaaatgacttggttcttttcttctttttttatatcagaatgaggaataataagttaaacccacatagactctttaaaactataggctagatagaaatgtatgtttgacttgttgaagctataatcagactatttaaaatgttttgctatttttaatcttaaaagattgtgctaatttattagagcagaacctgtttggctctcctcagaagaaagaatctttccattcaaatcacatggctttccaccaatattttcaaaagataaatctgatttatgcaatggcatcatttattttaaaacagaagaattgtgaaagtttatgcccctcccttgcaaagaccataaagtccagatctggtaggggggcaacaacaaaaggaaaatgttgttgattcttggttttggattttgttttgttttcaatgctagtgtttaatcctgtagtacatatttgcttattgctattttaatattttataagaccttcctgttaggtattagaaagtgatacatagatatcttttttgtgtaatttctatttaaaaaagagagaagactgtcagaagctttaagtgcatatggtacaggataaagatatcaatttaaataaccaattcctatctggaacaatgcttttgttttttaaagaaacctctcacagataagacagaggcccaggggatttttgaagctgtctttattctgcccccatcccaacccagcccttattattttagtatctgcctcagaattttatagagggctgaccaagctgaaactctagaattaaaggaacctcactgaaaacatatatttcacgtgttccctctttttttttttcctttttgtgagatggggtctcgcactgtcccccaggctggagtgcagtggcatgatctcggctcactgcaacctccacctcctgggtttaagcgattctcctgcctcagcctcctgagtagctgggattacaggcacccaccactatgcccggctaattttttggatttttaatagagacggggttttaccatgttggccaggttggtctcaaactcctgaccttgtgatttgcccgcctcagcctcccaaattgctgggattacaggcatgagccaccacaccctgcccatgtgttccctcttaatgtatgattacatggatcttaaacatgatccttctctcctcattcttcaactatctttgatggggtctttcaaggggaaaaaaatccaagcttttttaaagtaaaaaaaaaaaaagagaggacacaaaaccaaatgttactgctcaactgaaatatgagttaagatggagacagagtttctcctaataaccggagctgaattacctttcactttcaaaaacatgaccttccacaatccttagaatctgcctttttttatattactgaggcctaaaagtaaacattactcattttattttgcccaaaatgcactgatgtaaagtaggaaaaataaaaacagagctctaaaatccctttcaagccacccattgaccccactcaccaactcatagcaaagtcacttctgttaatcccttaatctgattttgtttggatatttatcttgtacccgctgctaaacacactgcaggagggactctgaaacctcaagctgtctacttacatcttttatctgtgtctgtgtatcatgaaaatgtctattcaaaatatcaaaacctttcaaatatcacgcagcttatattcagtttacataaaggccccaaataccatgtcagatctttttggtaaaagagttaatgaactatgagaattgggattacatcatgtattttgcctcatgtatttttatcacacttataggccaagtgtgataaataaacttacagacactgaattaatttcccctgctactttgaaaccagaaaataatgactggccattcgttacatctgtcttagttgaaaagcatattttttattaaattaattctgattgtatttgaaattattattcaattcacttatggcagaggaatatcaatcctaatgacttctaaaaatgtaactaattgaatcattatcttacatttactgtttaataagcatattttgaaaatgtatggctagagtgtcataataaaatggtatatctttctttagtaattacattaaaattagtcatgtttgattaattagttcttttggataatgttggtgtaatgtgtcattccttaagaattctactgttatataccatttttattaaggtcattgcaataaataaacatgaaaacatacccagaaaaacaaaactaatcacacacaaaaatgaagacatctcatagaatttacagattctatttaaaagtaaatatgtttcaaaggacttgcaaaagctatgtaagagatgtatgaattgtacaatatatgcattaacaataaagcagttgtacgtaaaatcccctcactgtcacccaacagtgacttataatccctgacacaaagtggttgggaaaatattctattgccagaaggttcacacgtatcaaataaggcacataatatataaatggcatctaatacattcccataccaagagatttataagaatgtgatctctcagcattgagtttttgtttcaataaatcgttccaattttttactgaatagatactgtgaatatcaaattcacaacagcatttatagaagtacaaggtataaagattggtaatctccctcgtttaaaagcatctcatttcattttttaaaaattcattaactatttggaaagt

SEQ ID NO: 10 - DNA, 32809 bp, Homo sapiens
  source 1..32809: mol_type = other DNA
gcatctaggtcaccttcagagccaggtggccattgggttaactagctgttgaactgaatcaaacaggctctgagccttgaagtcagaaagagccagagtggaggggtctggggggaagaggggcgctgaggtatagggagatgaaacctgcggtggaggcagcttaatggtccacctgtcacccaaggaatctctctgacaggggtctttgttagggtcacaccccaggagatggttgattatggctgagtccagcctggaatgatgggggttgggggcagcttgggtagatgactcagtaaatcaaacagaacaatgaaaggaggtcatgcttgtccatctgcattattgaagacagccataaatggccttaccccagagcgggtctgtcacacctggagagctgatctgacctctccaagacccctgcaactgagtgttctgggatctgtcctgcaacaagtgcctcgagatttgtaggtgggggcccagggtgctaagggattattttagcgggcggagaagggagggggtctgcagacgaagggggcaggttttgcggggcacttagggttctcataggttgtagtcacgagctcccaggtttggaaaactgcaatagtcactaagaccattcggggctgtttgggtctagcttggtctagacgggtctgaaactccgcaggacccacccaacaagaagtcattgttccaagccacgtgtcagtggtgggtgataccccaggatggaaggagttggtatgagccgactgaaatctacttgaaggtcaaaacggagccttatgtcttttgtgttccccagcggttagcccagtgccggccacaggggaagcgcgcaaacgaagtcctcgcgaactgaactgagagcagacaaaagcacgcgctcttctccaccgccacgccggtcctacccaaacccgcgagttatccgtattctccttcaggagtcatagtcaggccagaagagtgcggagggacggggcccgggaagagcagggaacccccagagcccgcagccaacgcggaggtgggcgagcgggcgtgcgcgcactcacttgccggcgcgagggagtgtcgggggggaagggagtggtctccaaaagggggaggggagaaggcagggggcggggagaagccggccctttaggacccggctgcggcggcgagggaggaggaagaagcggaggaggcggctcccgcgctcgcagggccgtgccacctgcccgcccgcccgctcgctcgctcgcccgccgcgccgcgctgccgaccgccagcatgctgccgagagtgggctgccccgcgctgccgctgccgccgccgccgctgctgccgctgctgctgctgctactgggcgcgagtggcggcggcggcggggcgcgcgcggaggtgctgttccgctgcccgccctgcacacccgagcgcctggccgcctgcgggcccccgccggttgcgccgcccgccgcggtggccgcagtggccggaggcgcccgcatgccatgcgcggagctcgtccgggagccgggctgcggctgctgctcggtgtgcgcccggctggagggcgaggcgtgcggcgtctacaccccgcgctgcggccaggggctgcgctgctatccccacccgggctccgagctgcccctgcaggcgctggtcatgggcgagggcacttgtgagaagcgccgggacgccgagtatggcgccagcccggagcaggttgcaggtaacgcggtctggaacaagtagttgggagaaacttggagggcagcggagaagcccgacgggcggctggaccttacggacggttttaggggcggcagagccgagaccttggaccaaatcaagggggactgttgctagcgggacgcggaagtcaggcccggggaggggaactggagttagagcaagtggaagagccctggcgactcatttggtggggatgaatggggagaggaaaggcatccctgctgccatagttttacccgcgtggaacggacccagacttctattctggaggagggatttgctgagaagggggccggtggtcagatccatgggagtaggaactcgaatcttatttggggcactgcttgaggacccaagggtcctggtgggatcgctgctgggaggcggaggctgcggggtgccagtgacaccaggaaccttggaagaggagagggagctaggaggaggcaccggcgtcccaaggggcccttccttctactggggttgtgagttggaaagaaaggacttggtactttgtaggatgagtcgcttaggagaaaaatgaaaaccaccactactatttctccatagggaacgagcagggctcttgctaccacgttgaatggaaaaataaaagggaggcatttggtaggcgaggggtctcctagcagctgaatcaatgtaacttaagcccgatcgccggctgcattccctggacgggggcagccaggctgtgcggtttcgcatctgggccccagatcgaaaggcaggagagatgcgttgccaccccccacccccgcccctatcttaaattgagggaccctttcctgtaggatagaatgaggacccgatattctttgttcaggggccctcatccctcccaaaaagttatttgagggacacaaccaggctctaaggaggttacttttttttttttttaattacgaaagcctcctgccccagtggtcgggaggaagctgtaattattttctttcctcttggagacagtggtgaaatgtgtccactgcctgctgctgttgttgaaaaagcaagtgagtcttaagtttctcaaacttaaaaagaaaaagtgaaaagaagaatgcttgctttttagcttctgaggtcttcgtaagccccccaccccatgcaccaacccgcctcccttctcctggtcggctattgtttttttccctgttctccttgagaatggaattaggggtcagagttgaaatctttcttctgtttctctagcacattttgctctggagtacagagcaagttagggcttcctctgagtggagcctctcagtccctttcttctccctcggctcgcttccttccctagcctgcctggaagttaacccttccctcctgcttctcttagaaagtaacaataaaaggaaaaagcccccacgaaatcctttcactgcggggtgcctgtcccccagtcatgcgttcctcctcatcacccctcgtggtccaccggggaggtgctgtgcagtttctgagctgaactctgacaaactaaacatcttcctcagcagggtgaactttttgaaaggcctctaagttcataggatggtttggctgctgactgttttaataaagtgtgtgatgcctttccttctttaacgttttacttttttttttttcctactcttcattcgtcttattctaagcctgaaaaccaattctacttcccaaatccctgaagccggttagggtctttagccaggaacagatgcggcagattttctcccattcacttttccctggagcctgaagggggtggggggtagttgtcaggggaggccagggacaatggggtgagttcatgggaagaatgtcactctggattttctgcctggttatgggactcccttccgccagcttgccccagctgcgttctcctccccagtgcccagacgccttccgcatttttgtgcaccggcctgggaagaggggatgttaagcaggggtggaaggtggggggtggggctggaagctgagtgggtgcactcaaaagaagtgaccaatgtcaggggtgcctgagaaagagtcgggctgggggaggggagcactttgggaaggagcctgagcttgcaagggtgcaggagaaaaaggggtcaaggttggaggcttttgtgtgggagagggtcttaggctggggcagagactggagtcactggatggagagtgctgggtccgagttgggtggggcctggcccagacggagatcttagcactgaatcctctgcctcccactccatctcccgtcccgtcttctctcctctccgaagaatgagcaccccccctccagcctgcattcctctttgagcaggttgcttttgagaattttaaacataccctcttatttcgaggcagcgaacctcagcagttccggctttgctgttagctcttttgtctttgttcccgggactcaggggctgtgttgctgctggcttcctggggatggcaaggatgcaggggtgggggtgggcacatcagtgggtggaaggagtgggtggtggtgagcctgtatcccacggggacgatgggctggggcttgttggttcaaagaaactgagaagtcagtcttgggggaaggggtgggtaggcctagaagaaaaaccgaaaggagaccctgagatctgcttgaagttgggactctaaaagcgtgtgtatgtgtttggtggtggagtggatagaagtgcctagaaatgtcagggtcgaggagggaacagggttgggccagtccaggccctttcagaattaaggctgggctagaccggttccttcagtatctccccagcctccttccctaccgtgtacaatgcaggggattatgctgaggggcggggccgcccgagtggccgtttggcccgctgcacgtataaaggtgctacagaaatgtgtagtcgttcaaagtcccagggcgggcagacaggcaggcaggcaggcgggcgggcgggcgggcgggcgggcctggggaggctctgcatctgcagttgccggggaagaataaaggggggaagaggcaagcactgatttacagtggttcagtgcagcttcaaaaaaaaagggggaaaaagcaatgggacttctggctcagagtactgctttgcatctcgtcttgcataaatttgcatactgagttcaaagtcgtaaaatagactagggaaatgagtgggatctactgtcactgattgttaacaccactaccttccacctcccctccccccatctggacagaactcccggcactagttctgctaagcagcgctgcctgagtcaccagtcaccactgttcatgaaagaaggagatggggaagcgcctgtctaggagaccaagcagggtgtggcagtgtgcaccctggcctcggaggcagggcctctctgaagggtgtggcccgacagattctggaccagtgaggcaagtggtgttctttctgacgcctggggctgccgttgcccatggtgggctgcagtttgaggaagctgcagaatccgggtcttctttgtttgaaatttccctactcagcttccacatatgtccaaaacatttactgtgataagtccctttgtgtggtggtaatgacccacttctttagacaagatatttgaggaaggttcgagaacagaaaaattctacttaacaatgttaaggaatctgagaaaagtggggcacaggagtctctaggttagaataggtggtagagcctggaaggagagaagaacccatctgttgtattcccgagggtctgcccacacaatggctcttggccaagtggactgttgagcttcctgtcaggcagagggaagagggaggcttagatagttctcactgttggcctacttcagcactgagcttcaagactttcaggcaaatcattccttcaggctcagaggctggaaattagttctataaaaatgccatggggccggtcgcattggctcatgcctgtaatcccagcactttgggaggccgaggcaggtggatcacaaggtcaggagtttggagaccagcctggccagtatagtgaaacctcttctctgctacaaatacaaaaattagctgggcatggtggagcatgcctgtagtcccagctgcttgggaggctgaggcaggagaattgcttgaacccgggaggtggagcttgcagtgagctgggattgcatcactgctctccagcccgggcaacagagcaaaaaaactccgtctcaaaaaataaataaaataaaaatgccacgggttggccaggcgcggtggctcacgcctgtaattccagcacttagggaggctgaggtgggcagatcacctaaggccaggagttcaagaccagcctggctaacatagtgaaaccccatctctactaaaaatgcaaaaattagttgggcatggtggcacctgcctgtagtcccagctgctcgggactctgaggtaggagaatcgcttgaacctgggaactggaggttgcagtgagccgagatggcgccactccactccagcctgggcaacaagagtgaaactccgtctcaaaaataaaataaaataaaaataaaaataaaaagccatgggttacctgaaagtaggagcatatccattcactcattatctgacctttgagggctgacttggagagaactgttctctggcccagtcatcaggtggcttccagttggttctcagctgggctgttcacacccatagaaaatgggaggtacgactgttcaatttctttttttttttgagacggagactcgctctcttgcccaggctggagtgcagtggtgcgatctcggctcactgcaagctccgtctcctgggttcacgccattctcctgcctcagcctcccgagtagttgggactacaggcacctgccaccacgcccggctaactttttgtattttttagtagagacggggtttcacagtgttagccaggatggtctcgatctcctgacctcatgatccgcccacctcggcctcccaaagtgctggggattacaggcatgagccacggtgcccgaccaggctaatttttttttttttttttttttttgagacggagtcttgctccgtcgcccaggctggagtgcagtcgcccagcctggagtgcagtggcgtgatcttggctcactgcaagctctgcttcctgggttcacgccattgtcctgcctcagcctcccgagtagttgggaattcacgcatgcaccaccatacacggttaatatttttgtatttttagtagaggcagggttttaccatgttggccaggttggtctcaaactcgtgaacctcaagtgatctgcccgccttggcctcccaaagtgctgggattacaggcatgagctaccgcacctggccacaactgttcgatattgatatgaggaagttctgaaaattctgcaccaaagggaatggagaacaggtaaatacctatgatgcctggcaggataggacagggggctgctgaaaggaatgaaggaggagcagaacaaaaaggggctgtgaggccacatccacatctgcttttgttgttggtccttcccgcgtactagaagactgtacccaaaagaaaagaaaaaagcttgttttttttttgtttgtttttttgtttttacaaagagtctgtgtatctgggaattgttatttttatttgtattattatttttattcatttatttatttcaagaccgagttttgctctgtcacccaggctggagtgcagtggcgcaatctcggttcacggcaacctctgcctcccgagttcaagcaattctcctgcctcagccccccaagtagctggaattatagacacctgccaccatgcctggctaatttttgtatttttagtagagacaggttttcaccatgttggccaggctggtctcgaactctggacctcaggtgatcctcccgccttggcctcccaaagtgctgtgagtacaggcgtgaaccactgcacctggcctattttaattttttaaggaactctgtatccagggagtgcatttcgtctcagcctttctctgtatgttcccgactccctcttcctcctcctgccttctccatgctgtcctctttgtccctgcagaccattctttcctttcccacacttctcccttccatgcctttcctactccggctcagctagaacacctgcttgctaggtaggtgggtggggctcaaaaatggtgtggaaaatatagtggtctcagcctcagagatccatccaatcagagggatattatgtaggtgactggagactataatgcttgaaaaaactagaatccttaaatgaggagggtggttgactgggctttgagggaaaggacttctataggccagggaatgggattctctcctataagagttagcactagcgctgcttctgctgaaaccaaggcatagaagcgggagatgatgtttgaattcatgagggaagctggtaacgtggttggcagatattctagatccctctgcttatcctgtctctggccagcaattcactgagatggcaggcagccgccaggagattggtggtgctcgcatctgtcctcaccctattatgaactggcttctgtgcctgctttgtctcctgtcttccacctccctgcgagggaggccgttctacctccattttgcaggtgtggacactgggtgactcagcgatacgaaaatgctgagaggctaacctgggtaccttcccactgcacccctctacctccgtggccgggtaaggagttgggctgttggtagagaaaagcttttgaccgtatcaaccagcagttttgttcctggacagctgccatgtgcaggtgatgtatggggcacagtaaatttctgccagatttcaattatgtagattcccttgactccatggacagttacaagtcctggagtttggggacctcacagttgtaagtagccagcatacagaggtcagtcaagagggacaagaaggtcacagggtgggcctcttgagagaagagctgctgggtagcagatgatggtgcaggcagcctggcctttgctgtcatattgaccggggttggttccattgcaaccctgtctcttaatacttcagtagttagtaaggctcttcctccaatgccagttccctgttggtaaaatagcaatgattttacctctttcccagggtgcgtgttggggtggctgtgataatggcaagtacctggcacatagtatgttcttgtacatgttagctgccttaatcttggcaagtgacatctcctccttgacaattagctggcctttaaaatgaaggccaaactggaaaggtcaggcaggccactcctctctggaggttcaaatcccaggtggtctaccatgtactaatcacccaaggtaagagatttcttgctaagcctcagttttcttatttgcaaatcagaaattgatagtatttcattgggtcaactacacagatgtgtgtaaagatataaactatatataaaacctaagctcttgcattttggtgatgagtcacttgatttccatctcttcacctatgaatggaaatatccatacctctccaacctactttcacagggctgtagtgaggtggtttatggaaagggcaatgaaactgtactttaaatgcaatacaaatgcaattgataaatgcaatataaatgcaactgataagtgcattataaatgcagtgcataaacggttggagaccatcgggcttgcattttttttttttttttttttagacagagtcttgctctgtcgcccaggctggagtgcagtggcgcgatctcagctcactgcaagctccacctcctgggttcacgccattctcctgcctcagcctcccgagtagctgggactacaggcacccgccaccacgcccggctaattttttgtattttttagtagagacggggtttcacagtgttagccaggatggtctcgatctcctgacctcatgatccgcctacctcagcctcccaaagtgctgatgattacaggcgtgagccatggtgcccgaccaggcttacatttttttttttttttcgtgagacggagtctcgctgtgtcgcccaggctggagttcagtggcacgatctcggctcactgcaagctccatctcccgggttcatgccattctcctgcctcagcctcctgagtagctggactataggcacttgccaccacgcccagctaattttttgcatttttagtagagatggggtttcacagtgttagccaggatggtctcgatctcttgacctcatgatccgcccacctcggcctcccaaagtgctgggattacaggtgtgagccaccccgtctggctaggcttgcattttttttttttttttttgagacggagtcttgctctgtcacccaggctggagtgcagtggcgcgatctcggctcactgcaagcccgcctcctggggttgatgccattttcctgcctcagcctccccagcagctgggactacaggcgcacgccgccacgctcggctaattttttgtatttttagtagagacggggtttcaccgtgttagccaggatggtctcgatctcctgaccttgtgatccgcccgcctcggcctcccaaagtgctgggattgcaggcgtgagctaccacgcctggcctaggcttgcatttttaatggattagttgggcagtgctctttggatccgatgtgctgatgtctaggactgactgtttttggtatgggagcttctgtctgcagtgtctagaatgggggttggagggactatggggaacagaggtggccccagagcttggacatctgggttctcatgctggccttgctgctcataagcaggtaacacagggttgagttcctcctttgcctggccagccaacctggaagacctagccttctggctgtcagtgtccagagctctggagaatccctgaggctacatctctgggtgggggaaggtcctctctgtatagatggccctccaggggaagtctgcaaggatccagacttcagaataatagagttaagtattaactaatccaagtggtcctagcacatttagcacatatggcatgctcaagggtgtgcaattgtgcagtcatcaaagccagtgagggcacccgatgccacccatttcctgcaccaggaaaatggacctttcacgtctctgaacctgggatgccagaatgcactgaacttgatcctggcaaataaattggaagctgtttccaaaaaagtgatatgttcttgctacattgtctccagagtttactcctgagttggggagcagcatctcattttgcaaggggctgttggattctccatagaaacgctgggttgggggagtgtcttctgagtactggggctctacctaatgtctgattccatctgtggtaccaaaaagccctctgaatggcagcctgtctgtgcacaatgatgccgaagactttcttttccagcatctaatgaggcatggccccaggaaagcagagaaggcggatgcttgagtctctgagggtcagaatacgcttatggtgaaatcagaactggggacaaggcaaagtactgccattgggaatccggggtggatggtcatgttagaccaggggtgtccaatcttttggcttccctgggccacttggaagaagaattgtcgtgggtcacacataaaatacactaacactaaccatagctgatgagctaaaaaaaaaaaaattgcaaaaaaatctcataatgttttaagaaagtttacgaaattgtgttgggccacattcaaagctctcctgggctgcaggttggacaagcttggcgtagactcttggagggctttcatggtggaggcaggaaaggaaaagccccttttacagatgggatagtaggtccagagaaatgtgttggttttcagttccagagtaatttcacctgctcccaccctccacactcacaaagagcaggctgggtgggatgtcgatttttttttcttctggagaatggaccatctccctatgcctctcctcctccttggctactaacccctgacttttccctcccccttctctggccactcctgttcctgaacaaagccagggaagatctgggctggctcagcccctgggtgtcagtcaagactcagagggagactttgctcaaaaagccagccctcccggaaagaaatacagagcaactgtagtcaggaatttttccctttccttttttctgtgggcggggggagggacacttggcagtctttggggcccttctggctgccagccccacatctcttcctggctgttgggaggcagggcaggagggaagattggggagaagggggcaggggctggcaaacacccagctgctgggcagtggaattcgtccataggccctggtggagttggcctctagtgaagcctggggaggtttctaattagtggaggacggtgaaggtgtggcagcctgtgttcattcaaggaactgagctctctgagccaggaagtcaaagggaagagggggactgagtcccggaaggccttggtaggtggggaggaagttgtacctactcagtgtgttccatcctattagaagttgagaagaggggggtgtttcagctggagtgattcaggttggactctgggggtgcagagagtcagtcattggcactaagcaaagggagccacctcaaagaataagtgatataacagtgataaacaaaatggctttgttctagacattgcctgcttctaccagaaagctcattttgttttttttgtttgttttatattcttcccctctcccacttcctgccccagctctgttttagcctcgagattctctccacttaacaaatgaagaaatcaaggctacattcaggttcaactgccaaggcaagcacagaccagattgtagtctgggtctctagattcttccagctctccatttcaccagactcacacatgcagaaaagcagtgaattatatactgcttcttggaagagctccttttccctgggaagtagtaattaaggcatatcttctcaaaaatgttcctttatgcaactctctaggaagtttcttttaggtctattgtctttacttctggatggaggtgccaagttgcctctgggtggtgaacttgttttctaaaatgcccgaggccaagaacaaaatgaaaatggcaattcccttgtgccctgtgccctgccccgtgacttcctgataatgctgagcagttgctatagcaacagtggtgggaggtgtgtcaggcaggataaagggggtgggggtaggacaggtttactggatcaaagaggcttttgcggttgagtagggctgctgacatgactgatagcctacttctagggaaagatgggggtcccatagagatggggaggggaagaaatgaagccctggggctaaaaggtcttgcttgagagtctttccttaggtgatgccaagtgaccaggtgccatctggttggttaacaacctgggcttggttttggccactggctacatgactttgtgcagttggcctccagctctatagttcatttatcaagtgtgagggggataagaaattaccaacctcatgtgcctgcttgataacaaaggtaaacatgcaagccacgtagcacagaaactcagataatcaaatgaggtagtgcaggttctgattcttgcaatacaggagaacaggagaatggcagacaccgcttttggacgttttctgattggtcatttcaagaatcctgttagcaggtgtgacttattcccttgagtgtggcttacctggacagctgccactttgctttggaaagcctccagtgagtccagagaggagtcaatggttgagagttttccagggggcagtcatgaccctcgatgccattgtgcctgtggttctaccaacagcttaatgaccaaccatgtcccatttatgccccagaagttaggactcccacttgaggctctgctggttgtctctggtttggttcactgcctcttaaaccttgagagcatagccaaggaaggtaattgatgtgatatactatcagacaaaattagtgaactttagttgaagacagagaaaagcatggtgacctatttaaagacacttatttatattcaagacacttcttgtgctggtttgagagtggccggcaatcctggacatctttgcaggttttaaatttctttttgcatcctgtgggttcaatttgtcaggcctggaatatctgagagcagagttatggggatggagaagaggacagtggaaggggagggtggtgggaaaggttggtaggattcagcctttgttacactatgggagtggggctgtgggattgcatcatcgaaactgaattgtttgaatgtgggtcttaggacaggcatgtgtccttcccatcccatctcatggttccacttgctcactgcctttcttgacttgctcactgcctttcttgactcttaaccactcttgctgatacgggctctagatgaggcaaaggccagaagaatgagaaaaagagttaatgtaacagggtactcttgggcaagtgctagatgctctttgatagtgtattagtctgttttcacgctgctgataaagacatacccaagactgggtaatttacaaaagaaagaggtttaatagactcacagttccatgtggctggggaggcctcacaatcatggtgggaggtgaaggcacgtctcacatggtggcagacaagagagcttgtgcagggaaactcccctttataaaaccatcagatctcatgagacttgttcactatcacgagaacagcatgggaaagacctaccccgatgattcaattacctcccactgggtccctcctacaacatgtaggaattgtgggagctacaattcatggtgagatttgggtggggacacagccaaaccatatcagatggtcaccttttctaatgtgtaaacattaattgagcacatgctgtgtgtcagctaggagaccatatgaaaaagcagttgtcataccacctgtaacacatgctttttcatgctggagggggacaagaaaagcattttagaaaggtgaataaagaagaaaccaaggcagcagagatgtatatacatcctatgcaaataaacaggtagctcttggtcactaaaagagcttaaccaagtctcttgggaagagcttgatgttaaataatgaagtcccttcatggtaaataaagttatgaaagtaagagaatcaagttggacaggcttctacttcttcagtcttggaagaaaaaaaaaagcagtgtcttccttcctaaaatgttaattgaggacacagaatgtcttctcacctgtccttcattcactaagtatggaaagctgtacggaagctaagaccaagtttatgtcttcaggaagcttctgtccttaggaggactaagaaaatgcacagacctacagctttttggcaatggactcaggctagagtgctgtgaagaaagttgggtcaaattaccagaagactttacccagcagtgcttttcttttttaaaattttgtaaaatttttatttatttatttattttatttattttgagacagagtccccctgttatgcccaggctggagtgcagtggcgtgatctcggcttaccgcaagctccgcctcccgggtttgcaccattctcctgcctcagcctcctgagtagctgggactacaggcgcccaccaccacgcctggctaattttttgtattttttagtagagacggggtttcaccatgttagccaggatggtctcgatttgctgacctcgtgatctgcccgccttggcctcccaaagtgctgggattacaggggtgagccactgcgcccggcccttatttatttgtttttttgagacaagctctggctctgtcacctaggctggaatgcaattgcacgatctcagctcactgcaacctccacctcccgggctcaagccatcctcccagctatggcagtgcttttcatcaatgcaatcaagtgacctttggacacttggaggccatattgttcatccttcttccttctcaaaaggtttctcctaagttataccagagggagtctgtgcaatttcttcccatctgcaatttatcagatttgttttgttctttcttatgaaattcttggatggcttctaacttggatctctcctgttttagtttaaggctgttttctctctggtccttggggagacagaacggctccttcccttcccttgggttttcctggcccttggacacctccaccactcagcaccctctggcagcaccaggatggatttgtcccttggctttcagttctcaggcctgagtcatcttgattacttcatcttccccttcttaattctgtgaccctcttctactacctttgggtcttctttctgactcatttccgagttcttcatggcatagagaacaggccaggagcccagggctaggttctgactggtggacaagtgaccctggtaacatcattgccctggccagtgagagtggtggtgaagtccgccatgtcactatcgtttggtcctcttccagtagggcttcagggacacttgaggttactgatagctctgcttgatgaggaagatgaggggtggttggtgtgagaggtgacttttttgtttttgtttttatttttgagacagagcctctcactctgtcacccaggctggagtcagtggcgcgatcttggctcactgcaaccttcgcctcccgggttcaagcgattcttctgcctcagcctcctgagtagctgggattacaggcatgcaccaccatgcctggctaatttttgtatttttactagagacggggtttcaccatattggccaggctggtctcgaactcctgacctcgtgatccacctgtcttggcctcccaaagtgctgggattacaggcatgagccaccgcgcccagccctgagaggtgacttttttaaaagaatatttaaaaaatgcagaaaagtacaaagaacaatgtaataacacaagtctaccacccagaaccgtgggcggttgatattttgtccactagcacgcagtacttacttagtgtttatctttcttagctaagcagacttgcatatatgtgatagatgactaatatgcctatgtttgcatatattgcatctatatgcatatatatgatagatgaccaatattgtccaaattgttttgtgaaaataaattgaaaatgcacataaaggttttattttccctcttaacattaggtgtctgagatctaatgttgacatccataggtcagaaatattctttttaactgctatatattgttcctttgtattaccctactggttttatttatccattcacctgttgaaacacttttggatctggttgctaatagtgtttttacaactacaagtaatgctgtaatgaccatttttgtacatgtgtccctgtgcacatgtagagagaagaattgctgggtcaggcacattcttcccattcccatagtccctggttcaagtttgaatcaaagcacagtctactcacttgggcctcagcaactaggtcaccttgccaggttcctggatggctttagttgaacccaaggagaaatgtttgggacccgtggaaaggagtgaaagctgatagtgctgtttgccatcttgatatcagtttctctggctcttcaaggacagcttggctcacggggccccagaggaccttggctgaatttgagcatgtttttgttttctcaaactgtctgcctcagccctgaccctccccaggtctttgcaattccaggaatgaccctgatgtttataaaggagccctttggagagtgcacaggcagggaggctctggagaggctgtgctgtttctgttcactcggggggagcccgagatgagatggcagcatgagggcatactctagaaacctccatgacgccaggccaggagaggcccctctgcatgtatccaagcaagcatatgtgtctgtttacaaacccatgagtggatgagcttacaaggctgtgtctctgcaaagcttatacccgagaccacctctcttgaaaagaagaggcattgttctcgcctcaccaggaagttcttcaggaaacagacagaacagagagaatcacaaaggcatgggggcctggcaatgccagagcttgagaggagaggtatcattcaacatacagattccttagtccttgcctctgactttctggggctggggtccagggctcttatttttagcaagctcatcagatgatttctgatgaagagctatgtttgggaacttctgactgggtctaattccctgaatttacagatgagtacactgacacctgcgcaagttgaacaccctgcttagcgtcactcagaatcccaggggtggtggtgaagtcaggatcgaacctcagtccttcctcccacactgggactgcttggccaaggcttcgaacccaaaagctggtgctccaaaggaaatccaaaaagagtttgcagagtggtttgacgtggaggggattcatcggccaggtgggggctctctgccctcccttggtggtgcatcatctcctgtaggtgttttgtcaccgtggaagggcagtagcctgtgcctgttgggcttgccactcaacagaggtcactgagctcttccgcactcagctgggtgattggcctgtggtagctgcaggtggagggagatgattctcagaagagaaaggccagttcttgcagtcgtgtgatcttataacctaggggacttgtggatctgaaacatctgagaaggctgtttgcagttatttactaccacaaaggaaactgggagaggagggaccacctgttggaaaaccatgctcagaagtgactggtctgttgctgaaggatggaaggatatcaggtgttgcatggctggctcccttgcaggaaggaagggctcagggggtgagaaggaataggaagggagtttacaatgaatgaagctgggtgtcctcaggagatagatggaccagttggggtgatgtcattaactaagctggaatgtgtcctgagtgccagcctataccgcgacagacatctgtgcacactattcagaattccagtgtgagaggccactgccccctctgaccttttgaaaaaaaaagtcaggggaagacacataaacagtgtcactcacccacaaagtctcaggacctcaggttaaactttagctaaagaagtaacaaatagaacccccattttaccttcctctttcctaacttctgcctccctccctcccaccagcatggcctgaattactaatcccagcctgttggtgggctgtgctattgaacatagtggccacatgctagatggagtttggtgaccaccatcagccatccaaagaggactggcttcaaattctctccaacaaaagctgtttcttgtatgtgctggcgtgtttatgagccctgtctcaactgagcatgcgtggtgattatgagctactgcctgacctccttccctttgtccctcccgctaaattctgtaaacaaagttaaagtaatttaggcaaattcattcttgcttaaggacttctattaaatggcagagagctttgatatttagcaatgtataaaatttgtacaatcaaaaccaagctttagaagaaatatgcaatagatcatattgcagaactctcatgactcattttttccatgcatttggccattctacaaatcagacattacctagccttcccctcacatggcagatataacttttatttacccattcaagaaattgattgttcacacattcctttttctttgaaacaaggtcttgctttgtcgcccaggctggagtgtagtggtgtaatcttggctcactgcaagaagtgaactccgcctgctgggttcaagcgattcttgtgcctcagcctctcaagtagctgggacctcaggcatgtgccaccatgcccagctaatttttgtatttttggtagagacaggtttttgctatgttggccaggctggtcttgaactcctggcctcaaatgacccgcccacctcagcctcccaaagtgctgggattacaggcatgagccaccatgcctggccaccacacatttttttctaaagcttggttttggccacagtgagagtttcttgggctgtcaggggtaaaaataccatgtcttgtttaacagcagccatcattttagcagaattttttttttttttttttttgagactgagctttgctcttgttgcccaggctggagcgcaatggcgcgatcttggctcactgcaacctccgcttcccgggttcaagtgattctcctgcctcagcctcctgagtagctgagattacaggcatgcgccaccatgcctggctaatttttatgtatttttagtagagacggtgttactccatgttggtcagactggtctcgaactcctgacctcaggtgatctgcccgcctcggcctcccaaaatgctgggattacaggcgtgagccaccgcgccccgccccttagcagaattgttaatatcatttttcagacttgagaaattagagctcagagaggttaaaggttctgactcgtgagtgaatctcaaagggcaaagctggcattcagggttcttgactgctgggttcattgctcattccagtttgttaatgaatcgtccactgctatggatattaatggagtcgtctagagagcagggggaggaggatggtggttagtatttattctgttgcccaacatatgtcaaggctaagtgccttttgggagaaattaacaaagacggagccatgtgagttttctgcccagctagggtggaaccaggaagctgcggccagccttcattcccctggcccattactcagaattaactcttgaaaggcttggaatgctattacctgttgcaagaggtgagttcattggaggaatgataagttcagggttggcttagatcatctctgtggtactcctctttcccaaccctgctcttagattttaagataatcatgagataggaggagaaggctggaaaatggtaagtctttcttcagaataaagaaaagaagaaagaagagagacacatgagctgttaggaagatgtgattgttctaggtagggaaaaggtcttatttcctcaccaggtctggataaggaccggggccacacactctgaggaggaaggaccagggaggtgcttttagattcttggggcttagtttcctaagaaagctttttaaggtctctcttagatcaggaatcaccaatttccaaatagagcactgaggtcagaacattgtgctaggagaaaatgagattttgtgggagtccgtttctatcaaacacaagagtttgatttgttgagtaagatcattgatgcaacaaacaggattacattggaaaaatgagctgtgccaaaattaggtctgaaaacctgggtatgaatgcatagaagtgaacaataaaggctctttctcttgggctgtcagcttccagctgctctccttggctaaattagtaagggtgggccaaaagacaatgtagggaccccatgataattgtgaagaatggcagagaggttgtttgctcctgtgaattcaaatggtagtccatctgggctcaggtttgccatctgtaaaatgggacaacgatccatttcttttcatcccatctgccttcctcatctccagctctcctctaggggtcttaggtagaaaggtgtggcagtctacccaaggcaggggtgggagttctcccctgcccttcaagcccccgactaatggctctggggttgtctgtcatctccctgacttctctttggaaaagcttgaccttctatatgtgaaatcccttgcttctgtgttgacccatttccctaggtgctatgtagggaagaagagctaatgcttactaatctacagggaaagtctcttcaggggccttggaggtttctatcaaccatttctaagcccatgagccttttccattgatgtcatttggggttgctaagaaccatttcttatagtgacctttgcaccatagcccatggagttgcatacataagtggttgcttaataaatattattctgaacttaactctcttaggcagaaaaaaaatacaacttggtatttttgttctgagggcaaattccatctctagctggagttcgacatctggaatgttttcctgtggattgttcttgcagcctataagaggatatatgggaagaacttggtaacatgtgataatgcccttgtctgactgaggggaggaggggcgctatgtgggtacttcctggtaggcaggaaatggaagtggctaagagctaaccccatgtgtgcctactgtgtgccaggccctctctgtagatgcaggcagcatctctttggatcctcccagttgcccataggaggtgcatactgtttgtccttttacagatgaacaatggaagcacagagagcttaagaaagttgtctggtgctgcacagctcagtagtaaaagcttgaattccaactcaggcagttagattttggggtttgaatctctttggctgggaacagtaacaaggaatgcaggcttttctcagcctacaatgctttctgatctctgcctggtcaactctttcatttcccttccctgggcttctacttcaagatgtggagttcagatctggctacgggatttgagtccagaagcctcttcctgagaaagaaacagatgctgatgcatggtcttgttaaaccatctttaaaagaccttgcatcagcaaaccaggcctgtggatttcttcatcttattggcatgttcgttggcactgctgagatcaataaagaaatggaatgaaacatacccttgaatccaggtggcctctgtgagggtggacaaacttgaccttcacctcagagagtttgggacatctcagggcaggaaatcaactttgtcctcctggaggaacactgccaagttgaccccagcctcctgggacctggagccagggttgcatatgacatctgtgggccctggacactgcctctgtgggcccttctataaaaacatatatttttaaaatttaaaaattattatttttcgtttttgagacaggggctcactctgtcacccaggctggagtgcagtggcatgatcatggctcactgcagcctctaacccctgggctcaagtaatttcccacctcagcctcccaggtagctgggaccacaggtgtgcatcaccatgcctggctatttttttatttttagcagagacaaggtgatatagttaggctttgtgtcgccacccaaatgtcatcttgaattgtaatccccataatccccaggtgttgatggagagatcaggtggaggtaactgaatcatgggggtgggtcccccatgttgttctcgtgatagcgagtgagttctcctgagatctgatggttttataaggagctcttccccgcttcacctggcacttcttcctgccaccttgtgaagaaggtgccttgcttcccggacctttggcttctaccacgattgtgagtttcctgaggcctccccgccatgctgaactgtgagtcaattaaagctctttcctttataaattacccagtctcaggcagttttttatagcagtgtgaaaatggactaatacagtcaggcgtggtggctcatgcctataatcccagcactttgggagaccgaggtgggcctcctgtggcaaggagttcaagaccagcctggccaacatggtgaaatcctgtctgtactaaaaatacaaatattaaccaggcgtggtgacacatgcctgtagttccagctactcgagaagctgaggcaggagaattgctcaaacccaggaggcggaggttgcagtgagccaagatcgtgccactgtactccagcctgggtgacagagcgatactctgtctcaaaaaaacaaaaaaaaaaaatcaaaaaaccatattttataacttgtttaataaatgcaaatgtattaatattatatagtaagactttttaacctaaaagttattttttttttctgatgttaatagaaataaaaatatgtcctaggcactgtgcctccagagcccaatggacaagttggccctgcctggcgccttcaggcccgaatgcacatttatctgtgtgtgcccctctcctttttcaaatgtgtttccagagattgcagagggccttcttctgaggattaatgttggattgacctctgggacgcagccaagggatggttctttggccactgtctgtggtacaagagggcttcgtgaagatggggagctgcaggctgaaagtcctctaggaagcccagggaagatcctgtgccctgcccccaagaaactgtggactattgtggctgcagatggaagggtagcggaggggtcttagccgtggatgcggaatatagtggagctagatttgctttatatggggcctggagccatttcagcaatgggctatcagcttacaggtcttgggctaaaagcctgactccagaagagtgattctcaccctgggcagttttgcctctccggggccatttggcaacgaatgaagacacattttttgttgttccaacctggggagggtgctactggtttctggtgggtagaggccaggaatgctgcccaacatctggagtacactggatgttccaggcattgagttcgggtgagcggctccttcccagaagctcatggatagaggtacacatgagctgcctttcccgtctccaaccagacaacgtgttgttatagccataatagagactgtctgccttcacccaagccacttcccaagagggcagagccaggtgagcctctgagcatcacagacttcatgtccacagattggagagttccagaaatggtggcagggtgcagtaccagcagactcccagagctcctgtcttctctaacaccctgtgagcagaatgcggtgtcgaggtctccagaaacccttcctgcctctgtgggcacaaagctgggtagggtgcggtaagcagggtgatgccaagggtgatctcaggtaccaagggttcgggtgagggcgaactggaggttcccacttcattttgtctttctcattttgatcctgagtagctgggagggagtcaaagggctcttctctatgttccaatgcaggtggtgggagtggggagactttctagctactttgctctcctcctgtgtttttggtgtctgcagagggcaggggcagggagcttgagatggtcccactcaggtctggggacagagaggcctgtctgggaagggcagcggggcggaggcagggaggctggggatgagtgcgtgcgctgcacggcatctgtgtgctgcgttagaggtcagtgttgagatggaaagttgactgaacctgatctcttctgtttgttgtccaggtttgtctgatctcagactttccccggcctttctctttcacccttttgtttctgacctctggggtccatctgaagagggaaggggggctcttctaggagggtgtggacctggtggcgctgaagaaatctgccgcacggtggggtgactcagcgtccctgttcttgggaacctggcaggcagctgagcaggggcactcactcagcctgcctctctgggtccctgggtgtctgctgctctccatgtggaggagcacagagggcagggcaggatggatgaggaaagctgtgggctggtgcctgggtctgattagacatggtggagcaggactccaacctagcctggaggcgcacgctaaatcggccttttcctgccggtggtgctgggctgccgggctgctggaacaatttaatctgtgctggcttcatttatggtacaacccaggctccccagagcccctgtgggaataaccattctaaataaaatatttttccaggtttcaggtttatacatttaacatttaaattttttttttcctggagttcaacaattttggataaaagtcttttaaaatgatgaataggtagaattttaattccctcccctttttgatgtcccaagatcatcgtcatcaacgtaattcttgttttgggctgtaagacatggaaatccccagggttcattgaggggcattcattgtttgccacacacaaaatgtacccggagtttgtatgttttgttttcttagtgtcctctttcctctctgccatttgatccttcttgctactgtccacatgcattccttctttgtcccttcaattttgttgactttcttctgcggtggccttggaaaccagacatccagaagccaggcttcctggagcagcactgttgtgaactgtctcgaggctcctcacgagatgattctcccctcatcttgagtggaaagtgccaacatttgaacatgctcagccgtggtaacagctattaggatgttcagagcttattttccttggtttccctgttctcggttgggttccatgctgctgaagtagtagataactggggcattcgtcattgtcatcatcatcagtgtaataaccagcagcaacatggatatagaatttctgtgctccaggtcgtcttttatttatttatttatttatttatttatttatttatttttgaggcagtgtcttgctctgtcgcccaggctggagtacaatggtgcgatctcagctcactgcaacctccacctcctgggttcaagcaattgtcctgtgtcacccttctgagtagctgggattacaggtgtgtgccaccacacccggctaatttttttgtatttttagcagagatggggtttcaccatgttggccaggctggtcttgaattcctgacctcaagtgatccacatgcctcagcctcccaaagtgctgggattataggcatgagccaccacacccggcctaagtcatcttttaactgcttttctttcgtcatactctcaacagctctatgaagaaagaactgttattatccagcaacttacagttaagaaagtagaggcacagagagggtctgtgtggggagaacacgtgcagctgatatagaaaatggcagagctggactttaacccggctacacccctttaaccagagctgctaccctggggacggcgtgctggcttctaaaggaagctgctgcatggcaagtgaagagtgactagttctcaggcttggagggaggggaaaggacagggacagacccccatcaaatgttccgtatgctggggcctatgctgggcacgccttgacagcaggtgctgctggagtgccagcacccggtgctgtccctggcatggaagaggtgccccctcccttagttggtgagtgagtgaatactcttgtatagagttactctaagaattattcatttgtgttctaagggctgtaagtttttgaaggtgcattaatggggaagaaaaaatgtgcaagattggttgaaaaacacccctctgcacccacccagatcttgttgggacctgcaggagagcagctctgggtgggagggtctggggctgtttgccgtgtctccaaggggttggggctggggctcggcggtgtgaggccctgcccttctggagcttgctggggcttgtctcctggttacagctctgccgtgcgattcagatcctctctgcctcctttggctggacgtgcctcagccagagtgagactgtggcaggctggagctgttttcaaggcctccttcacctaggctgtgcatacaaaaggttgaagaaagtataaggagacttaatggacgcttgttggacagacatcacgaagcttcatgccctgcagtaagctgaagctatgaagtcgaaggagtcatagttcctgttcttggggctctcagtataatggggttggggtgggctgcacagacagacttgggaacgagggaatgtcagtgtggcagagctgtagctgcctcggagcagcactatgggaccttaggcagggccaggtgggcaggtacacagtgacagcagggggatgctgggaccaggttctgggtggcgacgtggccttccaccttctcactcctttgccatatatgctgcagcccggtgatgtctgtggcacgagttttaaaaaactcaactaattggtgctcttccagaccacaaatgaagaactttaaactgggggttatagtgggagaatttgggggggtcccagaaaggacttaaacccattccatggtcagggcctttcaaatgcattcatccctctgtgtgcctctgttctgccccagccttgtaagagggtccctgtcattcttctagagccagacagcctgatttcaaatcctgaatccatcgcttttcagatttgtgatcttggtcaagtaactgacctctgtgagcctcaaattctccatctctgaaagttggggagggtgctagtagtacccacctaagagttagggtgaagctgaaattgatcatccatataaattgctcagcacgggccctgacaggccatcagcactcattagccgcgcgtcatctccaccctcatcatcattacggtccaggtggctcccggagggcacgtctctctgggaaactggacctggagcttttcttcccttcctctcttggcagacaatggcgatgaccactcagaaggaggcctggtggagaaccacgtggacagcaccatgaacatgttgggcgggggaggcagtgctggccggaagcccctcaagtcgggtatgaaggagctggccgtgttccgggagaaggtcactgagcagcaccggcagatgggcaagggtggcaagcatcaccttggcctggaggagcccaagaagctgcgaccaccccctgccagggtcagtgagggtcaggtctggtggaaggggtgggaggacaaggaaagtggggtctcagctggaggagggcatcctaataagaccctcatctggtgtgacctgtaggcaaagcacttttctttccacaaacatagttgtggaacatagttagtgacctttggggcagtcagtatctctggctgtgaaatagactggacatgtggtttcttgatgttctttccttctaaaaccttcacatttctgtatggttttggaacatagacacagaaaatgaatcccagagatgaagacagagtccctgaaacttgcttactcttttttttttcctccccctgagatggggtctcactctgttgcccaggctggagtgtaatggtgccatcacacctcactgctgcctccaactcctgggctcaagtgatcctccttcctcagcctcctgaacagctgggactataggcatgcaccaccacacctggccagttttgtattttttggtggaaatgtgggtctcactatgttgtccaggctggtctcaaactcctggactcaagcaatcctcccacctcggcctcccgaagcgctgggattacaggcgccagccactgcgcctggcctgaaactcagtcttgttgccatatgcttctctcactgagctacccctcccctctttcctgcccagctctgccatcttgggtcctggccctggttggttttatggaaagaagtggagcttgggcatggggtgggggcatggagactcgagacggccacttagatggatatttggaggcagagcccattctgacacatgaaagccacggtcacagaacaaggagaggagtgaacattcccatccatacaattttctttaggggctggggttgggcacccagcatggcagggagatccccgggcaggctgtcagaagtccctgtgccccctgctggctgtgtgaccatgggaaagtcccctccctgtgcagcagcttctcgctgagcttgcgtctggcgcgtgcttcgtgggcctgctgtcctcactccgggcgtcccctgctgtctgtgcgtgcagactccctgccaacaggaactggaccaggtcctggagcggatctccaccatgcgccttccggatgagcggggccctctggagcacctctactccctgcacatccccaactgtgacaagcatggcctgtacaacctcaaacaggtgagcatggtgccagcctgggccagagcagctcctcctcagccctgggccccagatgtgccttgtttctgccccacccgcctatgatcctctgaggtctgagctgagtgagagactcagactcctgtcaccatggggattgggatgccagctgccaggccggggagggtgcagctcttctccctgcctccgacgggtcagttgctggctccacttgcccagctgcccaccggcctcacttggagtccaggttgggaggaggctcttggagaagggaagtggcttaaaccaagcatctttgctttgatcggacattgacatcacctggagctcttcctgggcccgcccccaaattgccatgtggtaggtcaggtttacagctctaacaagcctcaggtgaggctcataatctgtgagccacactcccaccatcattgatttcaaccactggccgagttgtcttcatggtttcgaagtatctcagggaatgaagagtcaggcatggagaaatgccgttggaaagattctgaaaagccatctagaacaccattgtaaatacattggagaaaatgaagtttaagtaaaccactcaggagcccttctccctcctctatccaccttctgttcatatgcattttaacagtcactgtgcatatgcaatattttccctgtaacatactttttttttttttttttttgagacggagtctcgctcttgtctcccaggctggagtgcagtggcgtgatctcagctcactgcaacctccgcctgccgtgttcaagcaattctcctgcctcagcctcctgagtagctgggattacaggtgcctgccaccatgcctggctaatttttgtatttttagtagagatggggtttaccatgttggccaggctggtcttgaactcctaacctcagatgatccccccacctcgacctcccaaagtgctgggattacaggcatgagccaccacacctggctgggcattgggatttttataaagctaccctggtggttctaatggcagtctgggctgacctaaagtgtggatgtgtgaagccaggagatgggaacagggctctgctgatggcttaggggtctcacctctccacccccttcacccaggcaccttgaccctctaaatattagtttagtattaaaagctatttgtgtacatgccctctaaatgtaagctggctaactccatttggagctcagaagaaaaagtctcgatcacttctcatgggaaacaggctgtctgctgcacctttgggtaactaaagtgatttgcaggagtagttagcttatgcataaaacagagaccttggagtgggaatgcctggcttccatctcagctgtgcttcttaccagctgtgtgaccttgggcaagttgcttagcccatctgtgcttcagttttatcatctgtaaaccagggataataatggcatctaattcacaaagtggttgtgaggattaaatgactatatttgttagaaactgaactagcagtgtgtcttgcacacacagtgcctggcacacactgattttgattgcaacatgaggttttacacctgacgctatgactaagcttcagtatgctgggtcccccccactggatgggacctctttgaggctggggactgagtcttttcatcttcgggtgtcctgaatggccttgctaatcagaggggtccctgggcgggcagcatcagcatcacgttggagctcgctagcgatgcagaatttccagctctccagggacctactgcatgggaatctgcattttatcaaggtttcctggcgcatatttgaagttgcgaagctccacccggcagcgcatgggtagctgcaaggggtggctgctcagtggacgccgggttgagggacagaaggaaagttgctggctgcgggctcctccatgctcttctcctctctccccagtgcaagatgtctctgaacgggcagcgtggggagtgctggtgtgtgaaccccaacaccgggaagctgatccagggagcccccaccatccggggggaccccgagtgtcatctcttctacaatgagcagcaggaggctcgcggggtgcacacccagcggatgcagtagaccgcagccagccggtgcctggcgcccctgccccccgcccctctccaaacaccggcagaaaacggagagtgcttgggtggtgggtgctggaggattttccagttctgacacacgtatttatatttggaaagagaccagcaccgagctcggcacctccccggcctctctcttcccagctgcagatgccacacctgctccttcttgctttccccgggggaggaagggggttgtggtcggggagctggggtacaggtttggggagggggaagagaaatttttatttttgaacccctgtgtcccttttgcataagattaaaggaaggaaaagtaaagtgtgtgtcttttgcctgagtctttggggtcttccagggagagatgcagagcctggcctaggttggcctacccgccatcatccagctttgcccctggctcctaggaaaatgggaaaggccgtttccgtttcccattcatgaaatggcaataagaaggaatgggaacctcctgaatccagatcaaaccctccagtgcagccccaggactcccaggtgaggcttctgcccccatgtggcccctgcctggcgctgtacaggggcaggatgagtcacgggctggggatggggagatgtggagcgtgggtccggcttcctccctcgcacattcagataacactgcagtgggagataaggaacaggaaacgttgggattggggttgtctggcccctgaaataaaggaggctccctcctgcccagggtgagacatttccactcagcccccaaagaaagggagtagggctgtggttaactggcttattcataaggatgacaccactgagaattcagatgctgctctctaaggggcccaagggatttggtgtccagaccagcaaatatcaattgaaacaaagggctatgagtgtgtcaaggtgtgcagg

SEQ ID NO: 11 - DNA, 292815 bp, Homo sapiens
  source 1..292815: mol_type = other DNA
ttgtaaccataagtattcagggttgatactgcttttgaattagacagtgctcatatcagttgcataagaccaacctaaagtagaggatgaaatcttttttctgaacctttttcagaacgtaacttagtgaatatattaaaactaaactttctttgaatgggagtaatttctacggattaatctgtaatctcttagaccacacctaaggtaatgtagaggttgttgtatcataggctttgtgattagagaccactggatttgtctttggaaaagtcactcatgattctttgggctttggtttcctcatcttttataccggcttaataatgaccaccgtagagttgtcatggaagttaaatgaactcatgtaccataagtgaccaatacaatgattgacactcagtgatttatcaataaattaaaacatttattatcaatatgacagagaaggtgccgctaaaatagacaataggtttttggaagaggtgattaaatggatgcaaaatttatggattgtttattccgtctacctttgctgtgtcccctggttgtggcatacacacgtgtgggtataaaatcgtaaatcctatgtagtcgcgtagtgcatgcgcagaaggcttagacacgaaatgtcatttcagcaatgtgcctagagaagctctgacgccgccttggaagtaagtcgttgctgcctgacctttgggcgtctgggacggatgcctatacctgcacccagcagcactggaaggggcccaggcccttcgcagcacagcctatccccagaccgcttagtccttcataacatatatctccacggaaaagggtatttcctcccgtcagaaaaagcgccccagtctggtctgggttggtttttatttcacgttgttgcaagtaggcgaagtcccttctgtctcctcccttggggtaagtggaaaggagtccggcagggggcccgcagtggcctgcacaggggaactgggtagcgagagagttccaggcaattccgggggctgccccacagaagcaggtggggatcgacagtggctctccggcccagggaggagagcgcggtcgcgggtccctcccctcagcctggaggctgcagccgctcgagtcggcccgggtgggggcggggtgggggcggcgcggagggcacggagattacggcggcgccacccgggacatccagggccccgaggccctgggcggtccccacgcgagatcgcaaaccatgacaataggcagtcacccgaggtcaaataaaaacggagtgggtcccccgcgcgccgccgccccccgcgtccctggcggcctcccccgaggcccccggcggcctcacgagcccgcagtagccggtggcgacgtcgcccccgccccacctccctgcgcaagtgcgaggctgccggcagcgcggcgcacgctccggccgttcccggcttccgcgcaaaacttccatcctgtccacgtgaagttgtcgctgccttagagagggggaaagagctgcgggaaaagccggggagtgacgactgcggcggctgggcgcgctctctcattttcttttcttctcctttcccccctgtcgcagtccggagttttggctcctctcctttcctcctccccctcggagccggcttctccctccgccccgcttctcccccgcttgtgtacgctatttgttgtggggtggccgaaggggatgtcctgttttcaccagaggcacagcgcgaaggggaaacttcgacactggaaggaacgagaataaatacttaattacggacgcactgaaccgcggctgggacagacacttcgggaacccgaggcggaccgggcgacgaggtgagtgaccccttcttccaacccccgccccagggctcccgggggagcctgagttgagagaacccccaaactttccgggaaagtgcgcgaggctccgccggggacgccgagcgctgggtactgaggacgcgcagctggacggtgcgtgggcgcctgcgtccccggggggcgcttggaggccgggtgccccacgcctgagggcccgggccgctcggaccgcagcggtgctctctgccctagaagacgtccccaagccccaagggtcccttccgagcctgcctgtcccttccggggtcggcgcggagcctgcgcgtaacggagttcatccagcagtccagcgcgcggcttctacctgcaccccgcctccacctggcagaggcgcgagcatcggggtctcccccacatctttcttatgacgtgtattactttctgatgaccccctagatggtccaggcgcgaggatgctgacccagagtccttcggagggtcacaggcgcctgggctttcccggtgccgggtgcgtgtgtactttaaaggctcgcgttctaatctccaggcactgatcgggcttttcaactgcggcgatcccactttaatagtttttatgtggcgtggactgaatgtctcctgcagtttgccagggtcggtgaaattagaggcgccttgtcagagcagtcgcgttcattggctcgagtagcgggtgccatggaaggcttataacttctccaaaggaagggacctggctgggtagagcaggtttttctctccttccaagcctgctgggtctggggaggcagtggaacttgaaatggctcggatttttagcgtggtgaagcgaggtttggaagtagacgtgtgtgtgcttgttttattctgcgccgcacagcaacccgaactttcgtttggtagcacttgaaagagttttctccctttgtttgcgagattctgaacagctcggagcgattagggaatttgcggaccgagtccggtggcagagctggggcgaaagcagagagcgcaatttaatttttgtcatctcttccctgcttgggaggatagtgtttcccttcaccaccacccctctttctccttcctatgaagacaacggatttgcgcctggggtgagagtgtgtgcgggagagtggtgtggagactgtcctctctcaccgcgtctcctgcgcctctccccgccatcccgagcgggcctagagagtcattcatgaatcttaacctgagggcaggggaggaaggtgcaggtccctctgccctttctgccaaggtgcagaatagcgcccgggcgtgtgttttggttccagagcagttccacgtggagcaacttcgtgtgtgtgtgtgtgtgtgtgtgtgtgtgttgtgtacttgatctgtgaggaggtaacaggactctggtgttcaaacccagtgggccgttggccattagtttgctttcctggctgtcattacagacacttccaaaatctgatacctaagagaaccaacaggttaggtttcacattaagggctcatacttaacagttttctttctcccttaccccttttggcttggcaccctgggatcaacgtaattgttggagcgaaatacacctcctggaatatggcattttgttcctcttctcatctgtggccactttgtgaaccctcgggtgtttgtcagtttcagtgcgggctcctgcgggatttaggtgggagtcttaggagcgtttaacaaccgcgggctccccatcagcagcttctgaagtttcacttacacgtaggtgactgacaggattgaaagttgacgatggtttttttgtttgttgcgttgtttttgtttttaaaaccttagggaagggattgtacttgaattcctttcccgggtacggtttggttttaagcagaatcagtgcctttttttttttttttgtcttttaaaatattattggcaagcttaaacctgaagaaccaaaaactagagggggtggggagagaatccccccaaaaaatatttgatacgtgatacggagcgttttaggagactgcattcaaagacatttgtgtatttttaaaaataacattatcccaagaaacaaaaagcagtagtaacaagagacagattgttttgtgtggagcaagactgccagaatctgatttttatggcaacaatatcgaaagcagacataactacacccacatttattgttataaaccgtaaaaatagtttgttccacctgattaaaagttgtaagtcattcaaagttaaacccgtatttaggaatgactgtacaagaatgttaaaacctttgacagctacagctttgaaagcaacataattagatttgttgaaaatgtctcctttctctgattattagtgatatgtacttgtctttcataataagtaaaggtctacaatcaatatggttttcaaaagcctgttttcctttgtaaatttcctgaaaatagtatgtttcaatatttaaagaccattgtaactaccggttggttcacaattataactaaaataagatttactcagttgttttctttttattagtatttgttgaatatttgaaattgaatttctcatttccaaaataaaacgtctcacatatatgtatgtactcacaattaacacttcttttaagtagtaggcttaagttttaaaatttttaaaatcttaagatttgtataaaaaggagtgattgtataaaaaggaataataattattaattgttaagaaaaatagatgctgaatttcagagggtttctagaagctggagaaaaaaattgcatagaagtttcttttttgacgggtgactggcagatgagttacttgtcattttgctataatttatttttttcttttaataaggctctttgggttaaaaaaaaaaaaggaaaaacaagtactccatttctaagagtttcctttttatttatgttgtactttggggtgtaaataaattttacatagccacgggaaatagagtatccaatttcatgttttcagtctctgttgcaagctctaaatgactgatgtcgtgtctaaaaatatatttatatggcttgatatttcattttaacattttatgcactggaattagattttttaggatttttagaaagatagaatgacagaaagacgatgactattgttataaaaagttagaatgttaggaagttcacattctaactttcaaataatcaaacttattttgccccacaaaaactcacagtttttatacagatttcaagaagaggggtgataatctttttcactacttagttaagtgttaagtaatttaagaatgcaagtatgtattatgctttctcattatcatttttttctgttaggtaaatgcaagaacactggacattctgtaaaaacaggcccctctttataaggagtttattcaccatagttctgtattgcagctgctgatcatatttcatggagctgttaaagcacttaaaacctaaaattaggtactgtctggttgtaaatatttcagatcacttatttaagaaataaatagaaaagtgtgctatcaaaagtaggagacgttttgaatcctctcattgaagagctgaacaaacctctatcaaaacaccttcttcttttttcagtgagaatataatcttgacagtttctttttctaaatggatattattcttacatgtactaaatgctaaactcttataacgtgcctccctgtagagtatcttaaactaattatattcagaaatacagttgggggattttattacaatggttactaggtgaaggaaatcaacaccaggggaatggggtggcattgcagtgtacctgcttctctcatgaatttttcctgctaaactaagaaatgacatgctgtttcaggcatttgccctggagatgggtgagatgcaatatgtgtaatgctgcatcttatagttagatgtgttttaatgaaggggacactgcatagtcattaaatcatttttggagccaaacttggcgtcatttagcttgaacttaagtggaagaaaatgaacaagagttacacattcaaaagaagtacaagcaactttgattgcttttaagaggtttgaagactttgtaaacattactgtcactcaatattgcttgtggagctgtacattaatatatgctttggtgatatgtcattttacgcttgaaatttgtcattcttagtgtttctccatccatctttttattagtaaagagatactgaaaatgaacactactattcttactcccctaacccctttcacacctccagaaaaagagatgaaactgattaatttaaaatagaaaccattttgtgttatcaaaaccacatttatatagtgatttgagacagtttcagagagtgcacctctgagtctcactgtaaccttttttgtcattgaaaggtgctaattgatcttagggtactgacacaatagtatagtttgatatttgaaacctttcagagttggtctggcccttttcttaccctgagatttcagtgcatggatgatgaagaaagacaccattctaaaataccagaaattctcatttttttccaatatgaaatgttttaatacagtatgttcatatttttaaagcttttatttacatacagtaagtaaatttattttaacgtacttttggacagtaggagaaagacctatatgttctatcgtgttagaattttttagtttttttttttctgcacaggtagtttatttaggttataatttttaggcaaagtctgattcctattatcacatgaatattttcaaagtgaaattgcgttaaaccaatgtggaatagcttttgtatcaccaaggcatatattaatgtagatgtcaaatatgagagcatattttcttgagtatatttatatcctaaagtgtatttttaaataaaagtggtcactgtagtctttagataattacaatttggctgtcattattactataatattaatcactatcaccatcataacatcatagctagcatttactcaggaatttcgtgcaaaacactgttttaagcatttatatggattagctaattttaatcttcttaactatgccgtaaattaggtacttttgttattcctattttacagataaggaagctgagacccagtcatgcagcacagtggagccaggatcctaactccccagtatgaggccagcacccttatccttaagcgtgtgctgggctcttgcttccatcagtcatataccaccatttttaggtggtacctgaacattttgttttaataatacttatattttatgtaaggataactagatattagaaaaatttgttaaattttgtattaaacctgtaacttcatgggcaatattgtttgagacaagaccaaacaaagtattgaagtcaaagaaaaaaaattaagtatctgaagaaacgtattaagtaacagtgaacaagaatatggcctaaataaccacagtcatgaaggctggacagcaaatgactgaatttggagaaatgctgtattttgtaatgtttcccaattatcaagaacttatgaccagatcttttaaatatttaactaacatgtggaatcttcctttgctcttcaagccttatccaaattggttaaatgttatcaactttgtattttctttgtttttgttttttgttgttttaatgctggttttgaatctcaaatctgcacatttatgttgaaccaactaataaggcttgaagagttaaagagtgcatgatggacttctggaggcaggtttaaattataatggagctgcccatattttggaaatacattcaatttatctggttatcgcatgtgtaaggttttctgtaggtaaatacttttgctgttaataacctgttttacaattttatagtatttttccactgaagcagtggttttcatttttttatttacttatatactcagccacggatcctttcatttaaatggaaactgatgatatgcccagtagagaaatgtgccactgctctaggtgaagcaggatgtaggtagcctgagaatgactcaccaacagtaccctctcacggtggccgccgcctctggagttctctctaagtgtcttcaatgtatggaaactgctgcaaaaaaattcaagtcttctgacaaaaggggttaattcagagtacctgccctaacatgttcatgtggcatcactaaaaaacagactgtcagatacggtaaaatatctcccagtgtgacatatcagcagaaaggtgtgtctactctcccttctataacttgagtatccgtattaaccagtcttcaaattcgatttagcactgagaaaattaaaactgatcaaaatgttccctgtgtgtagttacaggtctgaatgaggcacaaaggacttgtacctgcaaaggttgactttattaattagaacatctttcctcctttaaagactgtaagaagaaacaccagcagtggcctaacttgacatgactttagattttcacgtaaattattgctactatttctgttatcctttccccctttctttttaaaatgaaagggacatttcttgtgaaagactacaattaaatcataaaaatttacattcatgtgccattaagtttaattctactcacaaaagcaacagtacagagtttgaaattctatccctaatcaagtaggtgtaccacataccgggagggctcattatgcacaaggtcatatatacaattcacagacctctgcatatacccaacggagtgatctattcattacatttcacctctgactttgaactccctaatgttaaaagatttgaaaagaaccgaatgttctgattaagagattgaatatttctaacttaatgttttcagtatgttgaaagtgatgatgacttgggggaatcagcagatctctacattacctaattcttttctcttacatttgaatgcaaatgtatattcatgtgcggttatgactcaagtcattcttgctaaatttaatgacgttgtaggtgaatcacattcagatttccttttgcaggttttccagtaatctaaaacaatgcttctagtaggtaacttaagcatgcaaacctcaataaacctgtcaagaacggcaattctactgttttattttgtttttttttttgtttgtttgtttttgcattaactttagttgataagatgatggtactgttatttttcttagttgactcatgaagaattttaatttaggtctagttttttcccttaattgttgactttagtttttaaaggtttcgttcatgaaaatggttagcaaagttgtgggtacttggtaaatgcttgttaaatgctttttccttatcagtgttgctgaagacttgcaaaattagagtgggatggatagatttcttttctactctgcatggctttgaagactttggagcttttattgtattcttatatttttacataccattcccaggaatatttagagagagaatcattgtaaccaaggtcacaggtctaatcctcaggtattcaaattagctttaggtggacaattgtcctatacacacttgttcgtattaatgttgtcataacaaatatagttataatatcttgatgcctctcctgggggcccattctggactgtgttgaagctgtctctaacactctctcacgtccctaagatattcaaccacatttgttcatgatttttatgaggtccctcctggacttaaaattccttaaaaaatttgcctgctgcctatagggcaaattccaaaattctctgcagggaagataacccttcatactgagaaccttgctcaggttgctcttctcatctctcactccccactcacaacaactctcttttccagctgtatggaaaactgcagttctcaaatacaccttggacatttccacttcatttctttgtgcgtggaatgctttttctctggaaaacatcgcccctctccccctgcaaatatcttttctactgctttttcaatacttaaggtgcaaaatgttagctcctctgataagttcctctgacctccagagtctgagttgaacgtttctttcttgttactcccaaagcatccagcttctacatttaacatagcccttgccacattgaaatgtaatagattacattagtctaaatcctgctttaagctgagaatttactggaaggattagctgtatttgtcatttctgtatcgctaatgcacagtgcactgctggacatatagtcagtaggtagttgttatattcccattgaatgaatgagtagaggtactgggaatgagagcagagattgtgacactgaacatcccctccttgagactgggaacagtgagaagggcagtgactattacaaggtgggactacctcaataacaacctcagggagttttggtataaacccagtagaattgcagctgtgaatccaggcttaggttatatgtatgtatacgtgaatgtagaaatgtgtttatttcttaccactgggaagtcagtgatctggtggataagccaaggatcctgaaaatctctggagatacggtaatttcatagtacttgaaatctgagagactcagttcttatagcacagtgagtaagtagaaagaatatgggggctgggcgcggtggctcacacttgtaatcccagcactttgggaggccgaggcggatggatcacctgaagtcaggagttcgagaccagcctgaccaaaatggcaaaaccccgtctctactaaaaatacaaaaattagctgggcgtggtggtgggcgcctgtaatcccagctactcgggaggctgaggcaggagaatcacttgaacccagaaggcagaggttgcagtgagctgagattgcaccattgcactccagcctggacaacagagtgagactccatctcaaaaataaataaataaataaaaaggaagaagaatatggagccaggcacttgggttcagacctcagctctaacattgatgagtttgtaacatcaaacacattactgaaacagattttgcttcagtttctcatgtgtaacagaacatgcctcatggggttctgatgagaattcaatgagtaaatatatgtaaatatatttagagtagtgcttgatatgagtatcagttgttattactatgattgggtatctttcattagattacccccaaatgttccagagatatttagagctgaaggtcttttttggggggctatggattggtgaccataaatggagtgtccagtagtccaagtataaattagatactgtgggtcatcttggcaaatctgaggttgtatttggagccttaagagaagacactataggtgctcaacagttggcattggccatttacgttgtgcagtaatatttcttttagtaaatatttccgcatacagtaatctagattgttttagagctttaatctgtgtggttccttaactactttaagcaattataagcacgttgtataatggtagtactttatagccaatatttaagtttcctcagttgcttttttttcagccgtaaggtaccaattatgagaatttgtataattttcaatagtcagtgggcctgatgaactcaatgtaaactttttaaaaggtagctttacatattgctatgattcttacctttagagagacagtaggaggtaattagtacctctacttacagaatttatcacttggattcagatataatggctatgtggcaggatggggctgaaggaaaaaggaaagttaaaatattaattgtcaaggtctcatttttacagcttgtctggaactgctaccaggtgtatctgtatagttttaaaaatgataacgttgtacctgaattcttcagtatatttaaataagagtttttcaagctctggttattgacattttgggctggataatttattcaggggatactgtcctgtgcctggtaggatgcttagtggcatcgctggatgccagggaccctccctcccctggtaatgatggtgtctccagacattgtcaggtgatctctgggagcaaaattgcctcttcttgagaacccataacctaagcagatcttaatatcatttgatatagtcaaaaaactcccagggctctgcataagaggattgttctatttccagtcaagtgtggagaatcctaataccttcctcccaagtttaaacatgaagtcaaacaattctcattagtctgtgttgatagattaactttgcacagaggggatttacagatacgcttcacacagattgaccatctccagcatattttccttttctggaaataatatatgagtgggagtaacagaatacctgagagagagtatgtaggaagtaaatatttatttgaatcatctatgtgtcttttcttccggttatttgtcagtagatagtttggatttatttttaaataatgaccattgcctttccatttctgtgggtaaatattcagtaataagaaacttttattttatttagtctgtagtgttagaaaaggtaaagttactgataatcacaactgctgaagattaaaatacttagtgagttaaattatttgtttgtaatgagaaattttcaaaagaattatgtgtagctttcagttgtaaccacaagttcaagatcttgagttaataacatttgtacagctagaagaaagtaaaaataaatatattacatgtaaaaactctcttttgtgaattctcagatccaaacttttaagttctcattctatgggtttgatgttgaccaatacattttatctatgaaatgatatataattgatgtaagtaaatttgactgttgtttgggcaccacccttacataatttaagcatatatgaaacacatttcaaaaatcacttgaacaaaatgggaacaataatgttcacataaagtgagagaggataacttttcccatatgagttttgggttgaatttcaaaattattctgaaatatgaaacattaatattataaatatatgattagactaattttcttaagagttctaaacagtagataatttttatcttattagaaattgcaaagatacttgaaattccacacttatgcattgtctaatttagtggttgtactttaattgtattaggtagataatcatggaagcagtcagactaacctggagatttcacatcatttactaattgatgcctaagtaggcagggtagaggtacttgaaaacacacacacacacacacacacacacacacacacacacacacacacactcactctctctctctcacttcctttgatttgtgctttaattttgaaatgtgtgcttaggtggcaaagggctgcaaatacagcatactgtgtttgaccaaaatatttttctgtttcctaggtggagaaggaccctgcatagctccttgttagcagagagataaagtgggtgctgggcaacccaatgacaggtggaggccgttctcggggagtgttgcggagaatttgagacaggggtttaaatcacagatagtggaggagagttctgcttcattcatggacaggaagtccaaagaaggcacagcgaaccacaggtccatccaggcactttatgccagatattgaacgggaagcctagtagccctttagttactggagaacggccaaaaacccagaggttctatttttccaaatgagcacttcctgggttcataaatcaaggaaccccacagagtattcaagtcacatatctcctttctcaggttgattctttgtatagtgggtcctaatgtggtaagacttacttatgaacattttatttcttataatgccgtcagtcacttccttttttctccctggcatattctcaccacccttatcctccatggccccaactcccttaacctacttgcctagtgctcttttgctgcctacctggacaaaataataataataataataatctactcatctttcaaggcttagctcaaatgtcacctcctctgtgaagccttccttaaggctcaggcagaattagttcttttctctgtgtaataccatagcactttttttggactgttaatataacagcagattgtgttggggctcattgtatgtgtctgtcttctagagagactatgaactcacagaggggagagatcatatctttttccctccgtaacctcaacacctggagatgttaatggaaattaagtgactgcactattccatgcatatctttaaagggcattaggtcccaaaccagacatctgcaaaggatgaatcctgttaactttttggaaaactggttctcttttgcttgtggtcatgaaagctggtttattggttattttctactccaacttatttaaagcctcatttgtaccaaagtattactgattttctagtggaaaaacaggtatgtccggcgtacattggtattttcaataaactgacatttaaaagacagatttctcgttgaggtttatggtcttcataattctagttttctccagaaacagaccttgatctctttggttgccttctgctaagatcgatttcatgttattttgaagaatttttaaccctcagaattatagatttcatttgagagaaagcatgttctgtatttgtttgtgtcagtgtttcaaaatgtgaggaccaaaaatcccagatgtatctattaaaaatatacgtggccttttgctgagggaaattacatcttttttttcaggcacaattgaggaagtaaaaaaattttgtttagtttttagtggtttgtttttaggcattttccatacaaagatgagcaagacccttcaaaaaccaccaatttgcttatttaggggggaaagtcttcctatgtccagaaataacattaaattaagattattgtttccaatagtttcaaaaattgtggttttattttcttgtatgagtcattttttagcatagtgcagtacatgattgcactacatcatgacaaagcatatttgctctgtgtttcagtgggtcactttatttatcagcgtcagatgatcagatcagaagagagatctagcctaacccttccattttgcagatgagatgttgtattatgtgaccttaagaccttaagtcatttactcagaacaaaacagaaaaagagaacttcatgctggggacacttcatgctcccaactctacatcttaccactttatttttatttgtttgtttctgatgaggagttgagaatgctggaatcagactacctttgtgtgtatccaggctctgccactcatcagcgatgtgactttggtcatgtaattttctgagtggctgtaagccatagttccagcatgtgtacatggggaataataacagtactcgcctcatggagttgtaaggaataattgaattcatgcaggaaagacatttggttcagtgcctggcccactataaaagctcaggttatagtttgaattgatatcttggaataaaatgctgagtgtatttgatattcagagaaggaaattcatgctgctaaagcaagtttttttttttaatctgttacttaaaaaatacaaataagtcatactttgtataacatattaaaacagagatgatagaggttacttttgagaggaatgaccagagggagcaagagagaggctttggaatgctgattatgtcttttatcttggtttggttgctggtcatgtgaaagtgtttattcaataaaaattcatcagtgatcaacttttgatttatgtattttattttactgtgcatatgttatactttcttaaaaagttttaaaaatacggatatatttaaagtaaaaagtggatttactcattcttattctctgcaaatcaggataagttgttatacttagatgtgcatcttttcaggcatttttttttctatatatgtgtaaatgtaataactatgtgaggatgtaatttattttcattttacatttttaactccaataggatcattgatttccttaaaacttgcttttgatctgctgaatcttgaatatttttccacatcagtacatatagttttgggtgcttctatttaatgtatgcagatatttcataatttcactgttcccctcttggtgtatgtttacattgcttccatatttttactattactaagtatgttgcactgatgatttttatgtctcttcgtacatctgccaaaaatatctgtagataaaactctagatgcaatattaatagataaatatgtatgtgcattttccatagaaaataccaaaatgctttttaaaaagatttatgaatcttgcatctttaaggtaatattctcatctttctcacaaaaaccaggtgcaaaaatttcaacttaattgaaccttgtaattatttgtgctctggttaagaaaaatggttatagtagtaaattccttgaaactcatatagtaatcggaatactccactatgattaggataaataattctattaacgatatatatttaatatatacatataatttatataattagtttctcacttaagaatatttagtattcatttttatgaatgcatattatatttacttatggttcgtaagcccaaatgttaggaagaaataaatcagtaatattatattcactacatttttttttacttagtaccatcaaatatttaccagaattattaggagaaatggttctaactctcatgtgattggtgagaacttatgaggaatgatatattgttattctagtaagagcctactctgtgccaggcacagttctaagcaccttacatatattacgatagctttttaacatagagatgaaaggtatagacattgtataagtaataaatgacattactccattataaatgtgttgataaatattattatacatgtcacaatatcagttgtgtcatatcaatgactaatttataaatctgttacttattataatctgtttaatgtggaaataaaagttaatgcatcaaaatccagaaaattgctactcaaatgctggctaaagtaatctgatagatacagttttaaaatgaagtatattaatcagttgattttgttgttcgatttttatatgtagaaaattctgtctccagaatattcagttcttgtagtttttggttaagattttgattaatcttcaatggttatctagtgcctttaaaaagtagtccaactcagaatagctaatgtattcctcttgcattgaataaatatggctatcaatttttgtggggttttttttcctgcagaatttcctgttattctaagggattcaataggacttacacatataaaactgaaaattatattactatggggaagctgctttgtgcctttcagaaggaactctcttgctaacttaagtactgtatgtgtgtagaatagctaatattttcctcccaagaaatttcatgtatgcagttaaaacactcttaaattgattaaggatttgttatatatttccagaagcactcaagagtacttttatggttataagagtagagtgcattagaatgccagaaaccaatgaatacgctagaggccttcaaaactgtgcccaacattcctgactttaccatataaacatgtggggtagcctggaaaaacaaaacaactctccttcccccaacgtaggtgaggcctgtgatgataacacttcaactggcttgaatattaatttgatacttttctgttagaagtaatttttattacttagcaaatgaaatggagaggtaataaatatgactgatatttatttcttgagtaaagtattattttacatcagattcgaccagttacctttatcctttttggaaaatgtattatacaaactaccacagtattttgcctattaatcagggaagctaaaaaacgttttataaacgttgaatcaaaactctcactgctgtgagggaattaaatttcaagcaatatactccttttatagggtgagaaactgaagtatggagaaattacaaggttttcccaaagctatacactgagtcatggcagacatagaaatctacaattctgtttgcctgattcaatctgaaacttcatgaaacgtaatttatccatttcctgacatgtgctttagaagtatatttccttatttgcttatttatgctctcattcattcattcatgcatgcatttatgcactattgactatttatgcactcattcactcattcatgcattttaaaagaatcggtgtttttgtatgatacagtcttgatttagggactatgaatattctgaatttatatatcttagatatttcttcaaagaaactcattgcatttttttcacgatcatgaaaaagaaattaagacattaagaaaaccaagctggattgtccaagtgtgggccatagcagtgtctatgggcagcacctgaagctaattcaggagtgaatctagtttgttcaagtgcttgaacaagaacttggctgaagccctggaggagatgcctggggcatagagtggggacgaggaaaaaccatagcactcttagagagtactgtaaggactagtatccacatctctaccacctgatgtctttacatttggttcagtcaagagagagggggcaaattattctgcttctcattctgccatcttcctgatttcagttctcacgctttaaattcttttttattgcgatctttttcttactagtttaaccagtattttcccgcgctttcttcaccaaattcgtttctttccatcagcttgcggagttatattttttgatcgaactgctggggttacaagtaatttgaaatgaggaaagtgtctagcaacttccctcagtccagtaccctgtaacagagacttcagagtacgtagtgatgacacctgccctcctccccttctatagagagtttagtgggcaacttttttggcttgttctcttgttcttttcttgcagccccaagacttaggactcagacttggtttctgcttgtatggctctccatctcttcccttggaagagttctttcccgggggctttttaggtccaagagcatttggacccctgggagatgaacttgtagattctacaaagaacatccaagtaccaggcaaaccacatcttcctgtagcttaaacgttagtcttttttcccccttcaattttactgaagaataatttagtctccaacaaacaagcaaacagaaccctagggtctttctcaaaggtgcattcttgctgctttgaagattatcatcattaatttctatttcagctaattggttttcctttacatttttatttgggtataaatgtgatactttggtatacattaatgttgatctatattaatacgtcaatttataattgagtgatttaaaaataatgatgcactatgacctagtttatatctgccttccacttaatgcatttcctaagcaattatgcttttgtggctgactgaattcccttataattccctgttgggaaacaaagatttttttttaaattgaagaagtggtcttgttaaccaggggcaccgtacttaacttgagagaaaaagattaggaatgacaggaatgacagcatactcctaatatctaacagcctgccatacatgtgaaagcaggacacatagcagggaagcataacgttgagttgactcaaaacaccaaatgtatttgatacattattttctgaattcttacaacttcagcccttttaaaggtctttggatttttagaagctataagggcagtttttggtaaaggtggccctacttctaattcagttacagagtcagtggaatgatttttatattcaaatgctacaggggaatttattgagaggaaaaagggtactcaaactttcctggatgcctcaattaaaaatctggatcaataaaatttcctcaagtgtcccaaatttaaggaaagcaaacagcatcttatttaattacattcttaattagtattcactatagacacaattcaaatcagtaacttggcattagatgaatcagatttattgccatattatataactctcatgtttactttgttgtgtttgggacttctccctccccataataaagaatatgactcacaggtgtcacaccttgattcctgagaaattaatgttatagaaaagttgtttggaaagatataattgtgattttgttattgtttttgttgtcattattttcttttatggggatgggcacacacttcttgagatttaccagatttaccttgtagttcataaattatttctttagatgtatctaatttgctgtttagtttgtttcttgtaaaaaaattcagtgaatatatttttaaaaacatttttatggttctcttccaggtttgtgtgtatatttttttcgtgtgtggcttttcaattttatttctgtttcttttgtgtctgttttaagtatacctatctagttttttccacattgctctgtagtttcacattcttagattcctaatttgttgttgtggctactctctactgtgttttgtttctttatgtagcttataattttttttttctttttgagatggagttgtgctctgttgcccaggctggagtgcagtggcacgatcttggcttactgcaatctctgcctcctgggttcaggcgattctcctgcctcagcctcccgaatagctgggatcacaggtgcccaccaccatgcccagctaacttttgtatttttagtagaaaaggggtttcgccatgttagccaggctggcctcaaactcctgacctcaagtgatctgcctgcctgagcctcccaatgtgctgggattacaggcatgagccaccgcacctcgcctgtagtttataacttttggtggatttctctcttagaaccctttgtactctgtggagcaattttgcattcccctggagtctagggatttcaaacctctaggtcagtctttattttattttatttttttgtcttgaggtttccataccacagatgcagtaaacatttagaatttacatcctgcaaggctgaagttttgatttatctcactcaagaaattattttcttttattctcttcccaaaggctccatcataggacaggcttctttgctgaatctgatggtgggccgagtttttctatcccccctccaataaatgtgacaaactttttagactccttcctctgtggattgcctcttgcttttgcttcctgcccatcgccataaaagaagataatgacaacaaaacgataacaacacaaatgtaaacgtaccttttcctcttaacttttccatatcatcaatttctcatgaattaaggtgtgatacctgaaagtcacatttgccttatcagttatattcatatagttcctttgtcttattgatgtatctctcatattatccctcatatccacttctccaggcctttttccaccccatgtcagtctagattctcttggcttctcacttggagcgtcacagcagcctttaagttgccacttaactccacttcttgttcatcttgtttgtcagaaatctgaagtagatgtgttgttgcaatgatatcacatgtgccattctttgttttgggggttttaaggcttttaaggttatcaaaatattgtaataaccaattgtcttaattgtattttgtacttattctcctgcttttgccaacttaaaatgtcttaacatcctgttaatactgaatgctgatgctttcctattcccttttctttcacatgtccgtctataattgtttattgattatatcagtctctcaacactaaaggcaccagtcacaactttgatgttatacagaatgacaactaaaaggtgacaaatgctaatgacttaatcgaggagttgttcatttttacaagcttacctgagttcagagaattggtaaaagactggtgacttttggctactatctaaatataattggttaatgtagctcctggagatcagtggttagtataatactcttagttgagcagggttaaaaataattacatttgtttaagttgcagatactactctactgtacagcaaagaatgttaaaatgttttaattcagtatatgaagagcaaaatataaaaacaggttttcttccactctcactagcaagcaaagtggtctgtatttttgaaatatattttttgtttaatcataacaaaatataatacaaatgttattaaaagccctattttatggatgaagacagtgatatttagagatattaattagcttttccaagatcatacatttatccagtagtaaaagcaagttttcacaagctgatctattttattccagtctttatttggaatcactaaacttacattccctttagcaaaactatcgtttacatttctagggtttcaagagttcaatgctaaacttgggagatctattcttaaattctctcaaagtgcaacacattttcctcaggatgatcataaagtcatcagatgtgtgttaagcaatatatatagtcatatactcttgatcaaaaaagatttccaggttacacagaagagagatttatggctgagaaaaaggcagattgcttctaagaaaggcttcaaatatattctcttctgacaggctacaggagagccagctagatgaaccattacagtgtgtacaaaatgctcagcagaataatttctcagtactggtagggaagagcaataggagtaacacaagagaaggaaagttggtctgcagagtctgccttagtatccgtaggggtgatgaggttgcttgggctaagaattctttcacatgtctccctgggacaattcactcagaattacttctgtgttttctaggctatctaggcaaccatcaccaattagcaaacacagacaaaatagaacaccttgtttggacctgcttgtcaaaaatcaaatgatcataatttataattccaggaagacttgccatcaaatgaacttatgacaaatgacccaaatgattctcacaaagtagagcttgttgtaacttttagcatgagaagtatgaaccccattatcattttttaaacattttgagttaaatggaccgttttgcaaagtccatgttctatctttatagacagctcaaaaatagctcagcatctagtttgtaggttggctccatttaaggagcttgaagtcaggcaaggtgctcattactattcatccagatctgtttcttggacacctcagctgtttttcttttctacggccagggaggagagtcatgtgcatagtccaagaatgtgcatctccatgtctctgaacagttggccatagaattggagctaggtgttctttgtgccattggctttcctctctggcatgcttcccgggtggttggagccagccagtgcctgatccagcttcacagacccagtaatgaaatttggagggcagagtaggtaactaggtacacaagaattgtaaagcaatgcccatctggacagttgatcacaactataacttctcaataatgtaacaggaatataaactgttagacttcctataccattgcaatataaagaagataattgaaggccgtgaagatagctcatgaaatgcaaggaagaattggacaactaagcaacaggaagagcagagaaacagtgagactttgggagtcacaagatctgggtgcttaaattctgccaggactttcttgccatcttcatttttgctcctctttctcggtcagttctcttctgtctttccctacagaccagcttttttccacatggatatgaaacgtttccaagttttatatcctaggggttttgccactgaaggaggactagcctgatattttcccagactcatatcctaaggaagatgaatcattagatccaagtaatttcgtgcaactggagaagacttgcagaccctacctaatgggttaagctggatatgcatctgtgggagggagatatgttttgtgggtataatacacatgcaagcaaaccttaatattgtacaagatacgcttctgaaaatttacttgcagtttgaaaatctggaagccaaacagtagaccaactgttgtgagtgttagaatttagtgtaatggcactttctgtgacccaggcaactctgcataggcagaaagattcttgaaagccccacccaggttggaaagggtctgtgtcagaggtcatgagtgggactcttcaggcacctgtttccccatcccaaggtctgcacatggactggaacaaaagttttggcagctgcaatttttaaatcttttcccaagggtatctagggatttaaagcagtagggaatgtactcccccactttcccttgttttccttttcactgcagcaagaggcaagtatagccctcagccctgcttgtaccctcacaggagactcatgtacaggtcccgggctctggctgcaggacagtgtgaggctgctgacagggcgagagaatggcgagaacaatggaacaggactgagggagaaggaagaggaatcgtccccagaactactacttgggacggtggtcccagggagcaaaaaacacccctggagccccttcaaggggccttttggcagagcggtactaccgctagacactgccatggctgcaggtaggactgattaaagaatttctgatttcttctgaggacagggtaaagctctgaattgtttttagagactgcattcagatctatatttcggaaccatcactctggttgcactgtgaacggtaacgtccagggagcaggactggagggaaggagaccattacgtggctgttgctcctgcccaagcagggaacaatgatggcggaacgcctgtactggcagagaggagagagaggagggatttggcgaacttttggaggtattggtaggacttggtaatatattgaatgtggagagcaaatgggcagggagtcagtgacaaagcctaggtgtttggctttgatgactggctatgcctgtgccggttgtgtacagaatccagaggtttagcgtttccatgtgcagcgtcggtcctcattccgcggcagcctggtgtggagttgagatgtgccccctccactccatgttcacgtttctttagccagagcaaagcatgcctggacaacctaggtttctttctttctttctttcttttctctagagagagagggtctccgctctgtcacctagaagaagtgcagtgtcgtgatcatagctcactgcagccttgaactcctggcctcaagtgatcagaggattaggtgtacttctctacaaacttctcaaacctccttcagcatctcattccttaagggaccttacagtctctcccacctggacggcgccatggtgcctcattccatttctgagaattgtcttaaaactttatatagaaacttgctattatgtatttaaatgagaagaaatgttgaatttgtaattctaattttctaagacaagagtttgtagctcgcttctagcagagttaaagaaaaagatatgatgaatggtctatatattgaaaactgcagaagaaataatctaatatggaatgggtgaggttgctacaggtggtgcaaaaataattgcggtttttgctgttactttcagtggcaaaaaccgcaattgtttttgcacaaacctaataatactgtttgaaattcatattttctggtggaacagtcttgagatagagcgggaactagattattggctcataaaacagcatgcagttactgttattccttaatcttatatctttattatgaggtaatgcttctcttacttgatttggagaatgttctcatctttttctcatatatttgtgaaaactaaatgttaaaggaattaacataacaaccattgctaaattatgatgtaaaataatactgtgtctgcaattagcctaggtagttttacttggttcaataatataaaatcactaggctcataatattttagtaccttagtgtgattggccaagaaaaaaattcacgttaccataaaaagtcaaaccaagtaacaattaatctaaatttcaaggaaatataaagcaaagtattatgttgtaacatggaacattaatgtaaataactccccaacagttcatgaaatagattctgaactggcagttgacaaagaaaggctagttttggcttaattctcttatgctccaatatctttcctgaagtcttatttaatttcacttgcgttagcaacagtactctgagatggtggtgcatctggaaaattatcacttctttcctgacaatcagtatctttaatttaattcttacatatgctttctggaaatattacattcatactttgatgacgtcttaaaaataatttcaatgtaataattatacatgtgtggtgagtaaattgtgttccgtaaaagatacttttaaatcctaatgtctgtatttgggaatgtgacctcatttggaaacagggtctttgcaggtatgatcaagttaaaatgagattttactggattaggatgggtcctaatacaatgactgttgtacttaagaggaaaatttgtacagagacatacacacacacaaaagaatgccatgtaacgttaggcacggagaacatcatgtgacaatgaaggcagagattggagtgatgcatctatgagccaaggatggccagcaacccccagaagctggggagagacaaaggatgcttccctaggatctccagagcaagcataaatctgccagcaccttgatttaggacttctttcctccagaactgtgagaatagatttcagttctaagccacccagtttgtggtactttgttataggagtcctaggaaactaatgcaacatgctatgttgtaaataaacagtagttctaattggtataaataataattttaagtatattttattattaggtcaggtgagtaggaaattgcctggtagactaattatcaatgttgtattttctgaaaagttaaaatattcccattcagtaaataatacttgtctaaaagtttctatctgtattgtcctaaaacaaagcaaaaattaaaatggagtcagttatgtacctgaaaaatgtgaaaaaggaaagaaaaatcttttaggaaagatttttcctaaaatggctatacatttgagaaagaattttaaaacttaaatcatctggctgtttattccatttttttgtttacattatcagatacattatgtatgatttcatacattatgtatgttacattcaatcaatatttattaaatacctaaaatttgctaggccttgagctaggtattgtagataggtaaataagacaaacattcccttcccctaaagagattattttggcatgaaatttaatctgtagaagaagtgaaagaggccatttgcagagtctaccatgaaaattcattttcactatctgctgtagttttacctgatcctgtgaaaccattttagcatcatttggttttactttgtgcttctgtattatattggagaaaataaaatttacttattgcacagacaacataaaattaattaaagtatgtgaaaccaaaaatgtaattgtttatcaaattttttttagtgtttcccattcatcagtatccaaatacattcttagttgtgcatccatgttagcataactaaaatgtggtgttcttattttttcatctaattcacaatcaacaatgttcacttttccactttttttttttgagacagagtctcactctgtcacacccaggctggagtgcagtggcatgatcttggcacactgcaacctccgcctcctgggttcaagcgattctcctgcctcagcctcctgagtaattgggattacaggcgcccgccaccatgcccggctaattttttgtattttagtagagacagggtttcaccatgttggtcaggctggtctcgaactcctgacctcaggtgatccgcccacctcggcctcccaaagtgctgggattacaggcatgaaccaccacgcctggcccctcatcatttttataattacttttttgatcattaaattatattatacaactgaccatgatttgtttaattaatccactgcaaagatgaggctgtttctagttttcattgtcatgaattattctgtagtttatattttacgagtttttttggggaaaggttacttcttaggaaagttaatactgggtaaaagggcttaaacatatttatagcttttaaagtacagagatggattgctttctaaaaatattagaaaatgacattgttactagcaaatggttgaggacaccgtgttcacagtgtttctgatattaagtattatcaatatataaatagtttgggcattgaatatgtatacaataatccaaaaaacatgtacttattgctcagatttatgtatttcagtaactgaaagtgaacgcttttccatatattagtttattatttacattttcttataataaattatgttctcctactctttcctaatcatttagtgggcatttaaacttcgcctcactaatttttatgtcctcagtaagtgtatatatagtcacatttatttactaaagatattttccataatctgttgtatccttttacttttggggctgtttttctttattcagaaatgcaaaggttttgctttgttttgtttatgtaggcaatgaattgtatggtctctttgcaatccaatgcagtatttttgccatttatgtaatccatgtatctatcagagtaagaaggaacatcagattttcctcggaaagagagcacttggctaatttgttatcaaggtaatgaggagaaaatggctctaaaggagaaggccagccacagttcagtctgggagaatagagggattgacatgaggtgggcaggtgtcaagtcttgggcagctctgtgcctgctgaccagtggccagcacttaactatttgcgtgcatcatttccaaggtgaacctgagccagagtggtcttggttacttgggccaggggacagttcacaggaccaaggatcaatgggtcagctaggaaaggataagcaagaaagggattagcaggaatcaggaccaggctgagatacccagatttgaggggaggaaccagtgcatcaggaggagaagagggagagtgggaggaggaaggtgggcgtgtgagctaagaggcaaaaagctgggcttttccatcacggtttcttagcctccacaccccatgctgtcccaacacaaaaacaccaccgtaaatctagaactctctgcctacttatactcctgaattcaaatgtgtactgctggctgggcatggtggctcatgcgtgtaatcccagcactttgagaggctgaggcaggcagatcacctgaggccaggggttcgagagcagcctggccaacatggtgagaccctgtctctactaaaaatgcaaaaattagccaggcgtggtggcacgctcctgtagtcccagctacttgggaggctgagtcacaacagtcacttgaacccacgaggcagaggtcgcagtgagctgagattatgccactgcactacaacctgggcgacagagcaggacttcatctcaaagacacaaaacaaacaaaaaaaccgaatgtgtactgcatcaactttcagtgtgcttgataatccaccatatcctatataccctcccttcccttttcatatgcagtttaaaattgtgcttttcttcgaaatgatgctttcctagttttggagtattttaaatcaatatctcactgaaactgtcacattatatacatgtgttattattattttctgacttttaagaatgaaaaaaacatacctttgatctgtgtctctgttatatatgcataaaacttttgcaatttacattttaaatttctcttgtaatttttaaaacgtcttgtgatctaataaggaaagtaacatgtactttggaaaatattgcaaagcataaaagaaattgaagaaattaagaccatgcctacttggatgcagtagggaatatatgactggatatatgtaaaactgagcaaaacatctactgatattgaatattcaataaactgaattggcatctgtattagggttctctagagggacagaactaataggatatatatatatatgggagtttattaagtattaacttacacttattattaacttaacttacccttaacaatcacagggtcctccaataggctgtctgcaagctgaggagcaaggagagccagtgcgagtcccaaaactgaagcacttggagcctgatgttcaaggacaggaagtatccagcatgggagaaagatataggctgggaggctaggccagtctctctttttcacatttttctacctgctttatattcactggcagcggattagattgtacccaccagattaagggtggatctgccttcttcagcccactgactcaaatgttaatctgttttggcaacaccctcacagacacacccagggtcaatactttgtatccttcaatccaatcaagttgacattcagtattaaccatcacaagtccaccccttgtcaacttgaacccatatacatctcctgagattatacatcatcttcaaataaagacaataataaggtcataattatacctaacataatacagctgtcctttgtataaccggaaatgcaccaatccccaacccaaatactattacataaagttaacaatatttaaatgccgatatgaagtcaataaatcttatgtcacatgataaaggaaaaggaaataaaggtattttcttagtacaagtgtatacatgcacaaacatgtttttaacaaaagaaggaggaaatactcatgacaattacagtccccgtttctgcagtggtctcgtggtcgtagatgctattgatgactaacttcttctactacccattctgtattccctttgccttcagcaagcacctcaacaggtcatgtttttttttccttgtggagtgacccaaaccttcattcctgggggatctgggccatttctagtcctgcctggattgggctgttgtagtttcccattgaccttaatcatagggcgtggtaatactaagagacaccctaatggatctcctgtattccatgcatactcttccttacctccgttgtggagtaatagactgatttcatcttgaaagtctgggtcagtcagtcaccccagccaacaatgtaactcccctcttagcctgttgacttaaaggtaggaggagcccaaagtgtccaggtggcaatgttaacatccagtttaatggaatcgttgttgtctctcctggtggcagtgttcctccctctggaactaagacctctaggccagcagaaggtaatgtcgtgggaacattttgctagtggaaggaaaaattttgctagtgaatcactaggggtgatggtgagtggtgccacttccacttccacccttgattcgtgaacccgtgaatcctggctatgggagagacagtgccatatattggatgctgattcagagcgtacatggctttctagagaactttgccccagccctgcaaagtattgtcacctagttggcattgtaattgtgacttcaaaaggccattccaccattctgtcaatccagctgcttcaggatgatggggtacatggtgagaccagtgaattctgtgagcatgagcccattgtcgcacttctttatccataaagtgagtgcctcggtcagaggcaatgctgtgtggaataccaagacagtggaaaaggcattccgtgagtccgtggatggtagtcttggcagaagcattgcgtgcagaataggcagacccatatccacagtaagtgtctattccagtgaggacaaacctcttccctttctatgatggaagaggtccaatataatcaacctgccaccaggtagctggctgatcaccccgaggaatggtgccacatcgagggctcagtgttgctctctgctgctggcaaattgggcactcagcagtggccgtagccagtttagccttggaggatggaagtccatgttgctgagcccatgtgtaacctccatccctgccaccatggccactttgttcatgggcccattgggcaatgacaggggtggctggggaaagaggctgagtggtgtccacagaatgggtcatcctatccacttgattattgaaatcctcctctgctgaagtaacccatttgtgagcactcatatggaatacaaataccttcctggtttttgatcactcagagaggtctattcacatatcttttccccagatttctttgtcaccagttttccaatcccgcttcttccaaatctctgaccatccagccaaaaaccattggctacagcccatgaatcagtatataatcacacatctgggcatttctccttcatgcaaagtgcacaacgaggtgcactgctcaaagttctgccaactgggaagatttcccttcagcgctgtccttcagagatgtcccagaaaggggctgtagtgctgcagctgtccactttagaggggtgcctccatatcatgcagaaccatttgtgaatcaggccctagtcttctcttcctctgtcaactgatcatagggaactcccgatgagggcatcgatgcagccttggggagagaaggtagggtggcaggagtagaaaccatggacatttgagccacttcctcatgaacttacttgtgccttcaggacctgctcaaacctgatcacatatatgctactttcatttgatgatagaatgctgctctgtacgaccttctttatggccagatgggtcagaaagctcccagttcatgatagccagttaaggttgcatagtgacttgatgactcgtagtcaaaccttcagtttccaccaaagcccagtaatgggccaagagctgtctctcaaaaggaaggtagttatctgtagaagatggttgggccttgctccaaaatcctagaggcctttgctgtcattcacctatgggggcctggcaaaggctccaaacagcatccctatctgctactggcacctcaagcaccattggatctgctggatcatatggcccaagtggcagaacagcttgcacagcagcctggacctgttgcagagccttctcctgttctggaccccaggcagttttatttggtaaagtgaatgattaaaggtcttggggccaaaatgatctcaacctattttcaaactttaaatgggttagaagcctggctcgctggcatagagctaggcgtggaatatgagtccctagtgggccacttttggtaagcagaactggcactgtgggaggagccaaacgctgggttaaggcacccgatgctgatgctcattagacaccccactcttgttaccaaaatctgtattagggttctctatagggacagaactaataggagatatatatatgtatatatatatatatatataaagggaagtttattaaatattaactcacatgttcacaaggtttcacaataggctgcttgcaagctgaggagcaaggagagccagtccgattccccaaactgaagcacttggagtccagtgtttgagggcaggaggcatctagcacgggagaaagatgtaggctgggaagctaggcccgtctctttttttcacacttttctgcctgctttatattcgctggcagctgattagattgtgcctgccagattaagggtggatctgccttcttcagcccactgactcaaaatgttaatctcctttggcaacaccctcacagacatacccaggatcaatactttgtatccttcaatccaatcaagttgacactcagtattaaccaccacagtgtccgattgtttctttagattgtaatactcttttccatttaaattgtattttcagaaatatttaatgtcagtagatatttcttgaaatgtgacttcaatgattatgttcatctatgatgaacaactttttaaatctaaattgtttttattataatcttttaatttttcttttttagaattagaatttgaacactagaaatgtaccattgcaattttaaattacatttcattgtgaatttgaacacagttttatgataattgaacatatttctgtagggcatgtgcctttctgtttgaattgtaagagctctttatatgttaagaacatcagcccattgtgtttggtatttgcacaggtatttctagatttttctgtgccttttaattttcattgtgtgtgtgtgggtgtgtgtgtgtgcgtgtatgtgttttaaaagagagagcaagagagaggattcagggttacttgggtttttggtgtgtcttctattgctactatgataaaatcctctatctggggatttaaaaatatatgcctatttttcttagagtttttcaactttaattcatttgaaaatattttaatgtatggtatggagtaaagatattttttgaatagttaaccagttatctcaccatagtgtattactaattatctttcccatgtttatttgtaatacattctttaacatattgtctttttaaggttggtcatgtattttatatttttaaaaagtgaagagtaattatttttcagaagaataatcatctgataattcaaattttcctgtaagagaaagttgtcatattaaaatgcacacttactttttcaaattattttatgtacttttgaaagtaaatggttaattcaaactcttgattgttaatctgtcaattgcttgatgacatacctccctcagtaaaatagtgtattaactgtttattgcagactgtgtttagtgcagactatggatatttttgttgatctgtagaattcctgactagctgttcacataccccacttttaccatgacactattttcaagatcattaattgcatcagatatggagacaatattagtggattttcttcttgagagaatgctcctacaagttactaattaccatgctatgtcttgatatgtttttgtaaccttacaaatgaatataaagataacatttttcccctttgtttccatcttcttgttttagatagtcatttttacttgaaggaagctgcttctacttgggagtggcaggagaagtgagaaaaccacatggaagactcccaggatttaaatgaacaatcagtaagtcttcattctcagttttgctgaaaaaagttgcgataacactactgaaaatcatacgtaaaagagatatattgtaagaagtaagtttttcattaaatatatttttaaatgatttttgaatttcctttcaaagaccatgaaacagacctttggagatttatgccgtttctgcttaatgatgatgtcacattttgatgtcttctttgattttctacttggaaaactagtggtggaaagactaaggcattgaaatcatgatgcatgttttagacagtctaaacaattttatatacgtcctttattattttatttcagatatgctggatattgttgaatatcagaagtataaaaggagaaagtatattattagcatgcttgttagtcattttaaggaggaaagaaaccatagttctcaagcgcttttagtattttactagattcattgcaggaccaagaatgcaccagcttgatggcaatccttttaagattctgatttcccaagggaaaggctctttgcaacctttcctcctagcttgttagtggtgtctctgtgacagtagttctttagcgtgtggaactagaggcagtgggagggcttccctcacacccccacgccttcacactactggaagctgtcgtttgagctgttttgcatacggacaactcatgatgttacaagtaacaagacgtgtgcttactgagagtgtgttctgcagtctgatgctgttccttttttttttttttgatgctgttctttattagcttatgtgatcatgacttgatcttacagtcaaatgtttctccatcaggcaactgaggagaagatacttatatctaataatgtggttctagaaaaggtatatgtgatgagtttaattttaagagggatgtatattctggtgccatttgaagttgatttgatttgctttctagtggatattttatttaaaagaaatgatagccatgcctttaatttggatagacatgaccatagtctaatttcaatatatattatattatgatttatttcacatggcatcataggaaaaatgtaacaaaaaattataaagtgaaaaaactatgaaatttgtcaaatggtagagatatattcctctagaattgtgtgacttctttagtaaggtaaacagaaatggctatgggaaggcaagggctttcaaacttttaaaaatgctggttattgactgtaaatattcacagcatggactctagcaaaatggaggttagtcatcatgactgttggaatcagaagtctgtctgtgtgactatggtggacttgaatagttagtagtaaatgttgacaacatcttagtggtgatgcttcattaaaagagtgctgttgatctgaactttgttctgatacaccgagggtcacatacaagtgtgtgtgtatatgcaaatgtttgtatgtatgtgtgcgtatatgttagagtgaccaacgtttttttgttgccagagactgaggattttctagggacatagaactttcagtgctaaagcacacaaagtttgggggaaaaccaggatggttgattctgttaattactatagggtgtgtatatactcaagaaagaattttgttttcaatgcagttgagaatatatttttatgtttaacactaaagtgatgtgttactaaagatgcgtcatatcaccgttcacaaaatgaagcacagaacttgtaaatcagatatggtgtccttctagtgatattttaaggtcatggagctcactatttctttttaaagtatgtttttaaaagtttccagtgatgctagtgagcagtcaggtttgagaacaagaaatctagggagacctgcctaccctctcagcttgaacccatttgctgaatttaaatttcaaacatgtagaccaatgtattcatctcataaatgtgcaaagagatgcttagaactgttaaatgacttgcccatgttggcatagctgataagaggcagagttaggacttcacacggttcttctgactccaagtccaaagctctctccataacatcgacgtgcctcattgcctttgggttatgcatattgtggcatttatagaaatgtcaaaaaaaaaaaaaaaaagaaaagaaaaagaatcagcccctttacatggaaataatttctctttgaatgaaatctttatatgattctaaattttgtggagtatattaatgtctcaaaatattttgaaaaatgatagcatttattaagatatagaacaagtaggggtggaattaaaataaagttcatagttagatgaaagtgaatttactcatatcattgctattttagttcaagagggattctttgggtgtagacatagcagtaggagtcatttgtggcacatgcacactgcatctagcattttccatatacttgctccaattcttaccatgtattccttcattgcaatttttaaggccactattttacagatgaaactgtgacctagagagtgtcaaggtcaaacagctgttaagtagtagagatcagaattaaaccaacttctctctggccccaaaacttttaatggattttgcatgatacaacataataggtagttaagggcatgagctataaatcagacttccctgtcacttaaatgctttaaaacctttgggaaattacttaaactcttcctcctctatggagtggaaggataataaaaagatgtcttatgaagtgcttgcatatgcctcacttagagtaaatattcaataaatgttagctgctatcacaaatacctgagatattcagatgctctttaatgagtattcattcattctagcatatccagctactgaccagacatcccttctgggagttcacaggtccacacactcagcaaatgaagaataaattcaccagctctccttccctctcccagcttttcttcctttgtcctttatgccagtattactcatctcagtgaatggcatcatcagctactgaatgggttatggcagaaaagaggtagtaatcctccttttcttcatcccgcatctcaaagctttgtccactcttaatgtttaatgtcattcaaagctattctttttgctcgagctcccttagtttagttgagatctttattttatctcttgactttcttgttttactttaagttgcttctagagttgtcttcctaaaatacaaccctgatcacatcactaccttcctttttatccttcacaagccctttcttgccttcaagggagaggctcaagtgtgtcatcaagcatacagagcttaatgcttatgtatttcttcagtttttttttttctggtttctgtcagtttctgaatctcccaggttctctgaggtctctatgcatttgcaagtgctttaatatcttcccagaagggtctctttcccaccaccaggataacctgtaattcttgaagactcagtttaggtttccatccctctgagcagccctctcagactacctcctttcttctcacccaatcagccctaccggtcagggccctctgcagtgtgctgccataaccctttggcatttacctctagcccagcacttaccctacaggactgaaaccctatctgtttacttgcctatctcaccactacattataaactacttaagagtaatggtggatctcgttcatctcgtactcttacttcttagcacgattcttgccagtgttcagtaaatgttttcttcaaaagcctttatatacttttagtgaggtatgttaatgagtagcctgacaaattttagaaagccaaaatgttattctcagagtagattactgactcatgaatttatattcgactcacttaaaaaattgcatagaaggtctaacatgaatttaactgcatatgaccaaaataaagttcagtccttcatttttcctctagtatatctggctcctatttggcattctggcttagaggatgtagagaattttaagagataagcaactaaaaagaatatttaactgtcataaatttatctgcagttctattttagagcttcataattctttagaattggtttaagttatagccagttacagcttattacagtttttaatgaaatgttcttgataatagaaatggctgtgaatgtaggttatacctagcaatagctcccttcatactgtgaaatatattgtatttttgtattcaagcagagaatgagcaatatttattatttttatagcttctataatatctttatagattgaggtgttttataatcctattttggtggtccgggatattacttcagattttttctcatccaaaacattcatatcaacatgagatggagtctggcctaaattaatgatcaagccagcaggtttgaaaagaaatatattttattaatgttaagtattgtatgttataaagtaaaacattcgtgaatattaagtaaaactaagtaaaatgaataagttacagcaggattgctttccctaaatgttatgcattacgggcctgaacattcatatgtatttccacattgttactaaatcaattcacttatattgttggtggcatgattttattagtgacatctaaattaactcacattcattggattatgttacatattgtatgattcattaatacttctggaaattcatttctgtgtccatttttaaatataatacatttatatttcaaagctattctatttgtatttatgaaacatattcatagacatgtttcagtaatacttttggaaatttgtttctacatgcatttgtagatatgaattgtatttatttgccttcaccaaccatgctaatttaataatgtctattaaaattctagagctagtttagtaaattaatttatttttatctctctcttgtttttccttagagtactcatgactttgattagtcctcagatctttgttcctagagatcctacagtattatctttgattatagccactagcccatctttaaaacgtgtactcctctctgtatcagatttttaactattgtatagaattgaatttgccattaaacttatcatctagacatagaacttcagaaccaccaatttatgacagtgtttaaacagtatgctagaatataattattttgattaatttttgtttaaatcatttttctatttttataatgtgacattatcaaaatcctaaaattctattattctgttgctttattcttgggtactctaaacccattcgaccagaaggcatacttgtactgaatatcttaaatatattttacttagatctaaacaatgttggattattctttttcaggatccaattctgtatcgaagaacttgacctttaaactagttaaaagaatcttgtttacaacatgtaatttttatactatccaactgaataacatacttcagagttctgtggaattctgttcaaagcccagtttcagaattactgttaactccaaattgccaattacagcttgactttgcagttatgtagtagagtacctaaactgaagttacaaagtagatgcgttcagtataatttaatgtttcataatcattaaggcttactcttcctttttaagcatttttaaaataatagtttttcttttttctttttttttgtttgagatggagtcttgctctgttgagtacggtggtacaatctcggctcactgcaacctccgactcccgggttcaagtgattctcctgcctcagcctcctgagtagctgggactacaggcatgtgccacggcgcccagataattttttgtatttttagtagagatggagtttcaccctgttagccaggatggtctcaatctcctgacctcatgatctgcctcgttggcctcccaaagtgctgggattacaggtgtgagccaccgcgcccggccaatagtagttttattgagatgtcattctcatacaatacagctcacccacttaaaatgtacaattcagtgatttctagggtattcatagagttgtacaattattaccacagtcaattttagaggatttttaatcagttgagaaagagatcccatgcccattagcacactccccattcccccgatgagcacccctcagccctagacaagcaccaatccacttcctatctctatggatttgcctattctggacctttcatataaattgagtcatacaatatgagaacttttgtggcttgctttttatacgtgacataatgtttgcaaggttcatctatgttgtagcatgaatcagtacttagttcctttttatggccaaataatattccattgtatggagacacttctatttatactctttaaatagaaagaatataaatttgcccctatgttataaggttacttctttttttacaattatgttttaagagtcatttctgtattctagataaaagtccttcaccagatctacacttttcaaaggatatctcccattttgtgggttgtcttttcactcttgatagtgtcctttgatgcacagaagttttaaatttgagtccagtttatctatttttttcttttgtcacttgcgcttttagtgttgtgtataagaaatctttgcctaatccagggtcacaaagatttactcctatgatttcatttctctatgatttctcatatgtaagcgagaacatgtgatatttgactttctatatctgagttgcttcagttaagataatggccttcacaaatacttactcctatgatttcttatatgaattttacagttttctatagttttagctcttacatttaggtctttagttcattttgagttaaattttattgaggaagatttttttttaaacttcaacttagtttagatttcaggggtacgtgtgcaggtttttgtcatgggtatattgtgtgacactgaggtttggggtatgattgatgccagcacctaggtagtgagaataatacccaactggtagttgttcagcccttgttcctcttcctccctgccacttccggtagttcccactgtctattgtttctgtctctatgtccatgtttcctcactgcttagctctcacatctaagtgagaacatgtggtatttgttttttttttcctgtgttgattcacttaggatgatggcctccagctgcatccatatggctgcaaaggagatgatttcattttttttatggctgtatagtatcccatggtgtatataaaccacattttctttatccattccattgttgtgggcacctaagttggttccttgtctttgctattgtgaataatgtctttctgatagaatggtttcttttcctttgggtctataaccagtagtaggattgctgggtcaaatggtagttctgtttaaagttctttgagaaatctccaacctgcttcatagtggctgaactaatttacattcccaccagcagtatataagtgtttcctttttttctgcagccttgccaacatctgttatgttttgacttgttaataatagccattctgactggtgtgagatggtatttcattgtggttctgatttgcacttctctgatcattagtgatgttaagcatttgttcatgtttgttggccgcttgtatgtcctcttttgagaaatgtctgttcatatccttttcctacttctaagtattttttaaactctgaatttttatatttttgtgtgagtttatgaagattcagcacaagatttcattaattattatcttctatgaacttctaaaattataatttgatttgttaaaattttttctcaaatcttagaatatatatgacacatatgtgtgtgtatatatatatatgtatatatggagagcctcatattatacagaatatggagtgtgattctatgtcatttgccaagatctggagactaaatattacgttttcttctgtcaactgtgatcagctggtaacagctgtttgtgttaagaaggaacctatggctacatccgagctcattggaaaagtaatgttatcaatttggtgtgatttgccatggacttggtaaggaagaagggtagcaccaagcctcataactgctaattctggtctggtgagttgtcaagatccctgggcagttggtttggatagtatagatataaataagagctatgatatctttgggtaccaaaagtcttgtgtctgaacttacaatttacatattgttatctaataattctatcttataaattaaaatgaaaataattcaggctgggtgcagtggctcactcctgtaatcccagcactttgggaggctgaggcgggtggatcacctgaggtcaggagttcgagaccagcctggccaacatggtgaaaccctgtatctactaaaaatacaaaaattagccgggcatggtgatgcatgcctgtagtcccagctacttagaaggctgaggcatgagaattgcttgaacccgggaagcggaggttgcagtgagccgagattgtgccactgtgctccaccctgagcgacagagcaagattctgtctcaataataataataataatataaaaaataaaatgaaaataatttttatgatagaaatataatctctttcatggtttaatactcttgagaaatagaaaacattaataaatataatgcaatttctgctataaggtatttaacaaatggaaaaattcactgatgaaaaaagttaatgttcatcgatagaggtaccatgccaagtgctttatgctactcatctcactaaatctcataacggccctttgtggaggctctgtcataacctcagtctgaagaagagtacactgaagcagagtttaggtatcttattcagggttgtgccagcttggaagctatgctgatggaatatgaactcacatagtctttattccagaatccatactgtttttttaaatatgtaagtttaaggggtacaagtgtggttttgttacgtggatatattgcatggtgatgaagtctgggctttcagtgtaaccatcatttgaatagtgtatattgtaaccattaagtaatttctcatccctcactccctcccacctgcccacccttctccagtggtctccaaaggtctccagtgactattattcctctctgtttccatatgtacatattatttagctcccatttataagtgaaaatatgtgatattggactttctgtgtctgagttgtttcacttaagataatggccttcagttccatccatgctactgctaaagacacgatttcatgttaaacacgtgaactcacctgcttccatcaacttacatgattttcagacccttcggtcaacttggtgtcatagagtatgtattaaaactcagcttgatcttagattatttatatgaagatcaaacaattttaaatattatggatttgagtgagaaatttaaattattccaccgtgattacagattaattgtacaatcttcattaatcacatatctcggctcactgcaacctctgcctcctgagttaagtgattctcctgtctcagccccccgagtagagtaactgggactacaggtgcacactaccacaacaggataatttttgtatttttagtggagatggggttttgcaacattggctaggctggttttgaactcctggcctcatgtgatccacctgccttgacctcccaaagtgctgggattacaggcatgagccaccgcacctgaccctattagtaaatattttaacaggcagtcataagatagcagaatgtatggaaattaaatgatagttcagcaaattcaattaagtaagcatttcaagacttcttgctatcctctagaaactttgctatactagggattaaaagatgaatataataattccttttctccccctaaattttagtttagcataagattatatgatactgtgtaagttctattcttagttcttttaaaacgagacttggatagctagtactaattcattcagtgaatatttgtggagcactgctatatttgaagcatggttttcattgttggaaatacttggtggacaaaatggacgcatattactgttcttgtggaacttatattcttgtggagggggagacaaacagtaaacaaatatagtatgtcagatagtaataagtgcaaggagaaaaaccaaataaagcagggaggaggataggaagtgcttgcgggcagggtaggtaggtgaagggacagctgtggttttttgtttttttttttgtttgtttgttttttgagatggagtctcactctgtcccccaagctggagtgcagtggcgcgagctcggctcactgcaagctccacctcccgggttcacgccattctcctgcctcagcctcccgagtagctgggactacaggcacccgccaccacgcccggctaattttttgtatttttagtagagacggggtttcaccatgttagcggggatggtctcaatctcctgaccttgtgatctgcctgcctcggcctcccaagctgcggtttttggagagtagttagggaggcctcactgagaagatgttgtgcaaagactcaagagaggcgaggaagtgagccatgtggatatctggggaaagtgggttttaattagagggaacagcaaccgtgaggtggtagcatgcagggtaggttcaaggaatagaaaggatgtcagcgttggtggtagtgatgggcagaacaaggcagaaataaggctgcaaaggtaatcagcatgggcatgtgagggtggaggaatacatattgtgtagaggtttccagaccattgtaaagcttaaactttttctcttagtgacatgtaaagcaattggaggcttttgagcttaggtattgtgtgatgtgactaaagtttttaaataacccactctggcttttattttgaaaatagactatgatgacaagagcagagactggctattttaactatccagatgaaaaatcaacaaagtagcattggaggtagtaagtcgttgcattggggttaatctaagatgacttgctaatgtatgggttatggattatgatagaaagaaaggtgtcagacataacttcaaggtctttgtgcagagaaatgggaaggatggagttggcattgactgagataggatgactggggaagaacaggttggggttgcagatcaggaggtcagttttagacatggtggcagagacattgctgtgttttcaccaaccctctttcatacccctcttttctggtgcatctcagtttccttacagttcagcagggccatataactagtgctggcctgtgaaatgtaaggagaagtgataatgttgatcattttgagaatgaggcagtgagaagccccagaatgattctttggtctctgtgttctcctagggtgatgaaggaggaggaggcctcagggaaacaatggggatccccagttatgaagaacgctggattgctagtgcactgcatggggggcacattctgaggggagtgttaaccaactaagctaattaaacgcatgtgaaattttactttctcttaggcatatgcatcagatgtaaccattgggaactcagggaaatgagttgaacttgaaatataaatttgggaggcttcaagatatcaatggtatttcaaaacattatatttgttgaaatctccaaaagaataagtctagctagggacaaattaaaaggactaagccctgggcaccccaggttagagagcaaggagatgctagtttacatagatagctgcttgatcctaatacccataagcaatgagttatgtcatagactgtatgtggccacattcagtgaattttcctccgaggtgcttctttccaaatttccaaaaacacatatccaggtcactgattactggattcacctctatcatagcagaaagagcaccatacagctatgaataggtattgagacagtattcacttatttgagatttccatcaaagtaaccaaaatcatgaaatatttagaagaaaattcagtcaacaaatagttttcattttttcattgctttgtttgtagatgtgatgtttctaggcacttcaggttaggaaagaattaatctaattagagttttttaacctcagcattgttgacattttgggccacaaaattctttgtagtgttggggaggggtgctgtcctgtacattgtgttatttagcagcatctctggtatctacccactagatgtcagtagcaccccccttcctttgttgtgacaataaaaaatgtcttgagacattgccaaatgtcttctctgtgtgtgtcggggggaccacagatctacattacatctgtttccaatgagtgttttatttatatacttactgcctgcaaaacatttgagaaagtttacctgaacatttaatataatatatactatgctatgatatattattgagggaactgcaatgaaaagccaaagacataatatagaatttaagcaagaaagcaaaataatattgcaaaggtaacagtactatgctatcagcatggaaaatgtgttcaataaaagacaagaaaggaattatagatagactaggttacaccttcaatgggtcttgaaaggcagaatgaatttgtatggacagaagagaacctatcctggcctggggaatatcaggcataaaagctcagaggtaccaaatataaggtgttaaggcctgcctaggactgtggctaggttagaattactgaatcagaagctctgtgggcatgaggttgtgaggcagtgggaaatctggctgagaagcactgagactagactgaaaaggactttgaagccaagctgagatgttttcgtgttcttgggtggccaccatgatgctcattactgcacaaaagacagagatgtataagataacagtcctgtatttctggactttctaacctggacataatggaaaattacatataatttttcggcaagagattaatatatttaaaatgatgttttaggaaaattaaccagatggtagggagattactgaaggtgggaaattgagagtacgagagatggcttaggggatcttgaaggagtttgcacatgaactccgaaaggcttaaaccaagtggccgggcacggtggctcatgcctgtaatcccagcactttgggaggccgaggtgggcggatcacctgaggtcaggtgaccagcctggccaacatggtgaaaccccatctctactaaaaatacgaaaattagccgggtgtgggggcacacacctgtaatcccagctactcgggaggctgaagtagaagaatcgctggaacccaggagtcggaggtggcagtgagccaagatcatgccactgcactgcactccagcctgggcgacagagtgagactccatttcaataaaaaaaaggcttaagtcagggattggcaatttttttgaaatgtgaaaacagctgtagacattaagtaaatgaatgggcatggttgtgttccagtaaaattttatttacaaaagcatgtggcaggtggtgggccatactttgacaacccctggcctaaacttatgacatgatggtggaaagaaagacatcgagtaaagaatcaataaaaggaacttgaaaataaagctttgctgaacttggtaactaaaccaaatagagttagtaggaggaagggcaaggggtggacttggaggaggaaaaagattatcctggagtttcaagcctagaagatgtcattaatagagtttaggagcaggtatcaatgacttaaggaaagagaaagatgtgtttgaattgaggacttttgagaacatgttaaactagcaattggtgaaggcctgctgggccatgggggagaagtctggaacttctccatgctcatgtgtggaacagaacagtattttccttgaggtgatgcttttaaggggagatggtatctgaggaagggcagaaggatgaagataaacataggagaccagcatgtttatgaagtgcaaggaggaagatgaactgtaagaaactgagaaaagatagggaaatgagaaccagtaggataatagtgtcttcagagccatggccaagctaactctcaggaggaattggtcattactgtctgacgttccaagaggtcaagcagaatggcactgagaaagagattgtttcatttcttgaccagaaggttactggtggcccttcaaaaggaagtcccaagagagttgtggtaagaaaaggcatcccagagattcaatttagagaagataatgaggaattaagagaaagtttgtaagtttaaagaagagacacagaacagcaattgaatggagcaccttcttccaagaggaggcttgcttgtgtttgatgacagattttgagaaggattcagtggagaatagacttcaagttgttgcagaagggatgatgaggttaatggagaacagaaagaaggtgaactagctggagaggagtgatacactctgactctggaaatttaagagaacgtggatatcctggaattttccaaagtgctcttccttaatgcattctttttacaggaggcaaatacctttgtccaggcaagatcaggaagtagatggaagcagggttagaattaagggcttaagtagtataaagaagatttgcagtagccattgttataaaaaaatagtaggtgatctcctagaactcaatacttggattattgagacatactcagttttcttttagcaaaatatctctcattgactaggacttaaggctatactgctagcattgaaaatttattttattttgtattgaaacaattaagaagataaaggggaaaaataaacacttctaagattaatgaatatgcctctcgttatctttggaagaaactatgtggcctgttttccttggatattagcatctccaacaaatagaagagcctatgttcataaatatgattgggtcaaatttgacaatagagtcagactaaagttagctgacattgactttaaaagtacttgcctcaaagtacttgaaattcggtggcttcatattttcttggattagggattgatttctctgctcagacttcttcagaatacaaataactttgtaagggctaatctgttaaaaagtttgtgttagctctcttggcaagagctaggatagcagggtagaaggagaactgcgaaaacaaacaaacaaaggagagttaaaaatgaaagggcaaacactccctacttactttgctttgctttcattattttgcctaaactgttgtggttgacacacgtggacagtagattataactagattctgagccccagatgcccacagctaacacttatgtaatgctcattgtgtgccatgggctaagtgctataaatgaatgtatatataatataaatgcattaactcatttaattctcatgacgatttgatgaggtgagctctatctcccagtttctagatgagggattgcccaacttgtgggtaggggagcagaatcttaccccgggaagtgtggcttgagagttggcaattgtaactgctggacagatttgttagaaaaataccttatttatttttcagtcctccaccctgacttgtgccttcatcaaaaaagtttgaaatgttttcagttcatacataagaagaaaaaaatttgatatgggtttattttcccctattaacattttctttgatttacagtctgttgtatcagacatgatgtgaaaggggtagacaacaacctagactatatttatttttcaatctgctaatgtgaagaaaaaaggggaggagttaggtgagatttggagtctgtaatggtttttctacaccagggtttttagggagattctaaatagagcacactctcatttgtttcaattaaatgccttgcccacttaatctccttttcgtattgccatgcttgatacagagggctccaggactagtttactaggtggtaggacctttagagattatgctgtccaaatcctttacgttagtttaacagatgcaaaaatggagactgaaagggtttaagcagagtgagaacattaacaacaacactttgttttgaaatgaggttttttgattcgttccaaccttcacacaaatttgaaagcatacttttgtgtaagtttcttaatatttggaaattaacatcagccccagcctccctgaaaactgtggtgagttattactatatataaagattaatgaaataattgcttttgagtttctactacaattggtggatcagggtcatctaaaaaaactcaagcaaaggtgtgaaaactttattttgcatgtttgtttttctcctagttattatttactgctcaatcagcattaacagaagaaaaataatttggctgctaagttactgtagtataggatttcaaaggaccttattagagagagatgtaatttcatcccctgggccttttatgtaacagagtgcatatttgggttacttcattttgacccggaattaaacaatagacttaagttgtattcaggaagttaaaatatttattcaacaaagatttaaaagcattttacagttatttccaaattcctagggatatctttatagctttattttcttattttagctaactcttgtctgaatagctccttcatcaaagaattttctgtttatttcataaatcttataagatgtatgtatttgataactactgcatataatcagtgttttttaaataaacatctttggcaaattgatataaacatgattttatatattgcttaactacatatataggccatagtttcgttatggtgctcagtaatgaaaaatatggagtagatgttacaagtggtaataaagctaatattaaagaaagtaaggctgaagattcctaagttcaaaataaaaaggctttgcagcttcagttttaggagcgttttcataaaatgaaagaggcaaatagtgccaaggtgctaccaactccagcttcctagcaatgcaaggattcttcctttagccacttataaaagtggtcacctcagctcactctctgtaggactgactgcttcccctttcttctgtgatttgcatagtattatccacattgattcatgtttggggtgattgtttccatcctcactgtgagattatgaacttcttattttctttccatgaaataaagttttcattggctgggatcccagctagactgatttttttttccttttttcgtttgtttgcttaatattttagattttgaaagtccttcaaaattaaagtacttgcatactatcctagggcaataatttgtgactgaaaatctttgtgaaaaatcctaagaggtatttaaaagtttatgttaagtaagttgttatcagtggttaagaggcctttccagagtgcttgtccactggacatttatatggaacttactgtaatagcatctgcttaatttttggatgatgcagacacattccgttgcacaaaattactctgcaaaagtaagtcaggaatcactcatgataatttcaggttatgcatacttattttagaatgttgaatcaatttgctaatcttcaaaggagtaaattttacaccaattgcgtctggtttaataggcttaatattttcataatgccagagtaatttttcttcttagttttattcttgcataattgttggaattatatgtacattgttttaattcttgagcccttaatatcaaatagcttgagcattaagggaattaattgaaggccttaggatatgggtgtttattacatgaacatggattgaatgttgatgttgggattttaaacgggaagttgctttgctgttagattccatcacattgaatgtatcaacatttatacaaggaaattgggaagaaatgtactctcagtccctggttgctctttgtttgtgaaaagtaacaaatttaggagttgatctaaaaggaaaatttttgtgttaatgtttttaaaacattaaagcatctttaaacaatatttttgtctcagatgtctagatgcatatttatacccccagaaatttgaataagtctgtttaaatatttttgtccttttcctgaatgtcaaccctttggcagagtgtttatatgttaaagcttgaagagcaagtagagatcacacagtccaactctgtcatttcatagatgtaattagagtaaactaaggatcagtctgagtataatgaagtataaaaaagaacttgacactcactgtcacacaacacagttgatagaaatttgttggtcattcgttggccactggtcatcttccccccaggggtctctggcttgcaatgtattcccgtggtctctttctctgatcccctcttggatctcctcctcttccttaggtgttttgaatttctcttctttgtcttcagcatcctcaattcctcctacttcattttgcagaaatttagctgtctttgttatgccctcaccatccaatgtatgcccagataactgtgcctttttcatccaaagatccactaaaagatgacagtgtttttattttttatgagacacatcacctgcagaatgatttttttttctattacagttgacacatagagatgcattttaattcacatgtagacaaacatttgatgaattcctttttttatatatatacttgggaaataggtaaattttctgctatcaataatctctcagaaacacttgatgactgaatactatagttaaaaatcatgaatgagaataagaaaggcaacatagtattttaggagggacattatcccactcattgaattaccatataacctgtcatagcaccttaactgctctgaacctctttcctcgtctataaccttcatcctggcctgcactggagtcagagttaaactgtgccttgtaaaacataaagttctttataaatataacagttagagcctgggcgcatgcctgtaatcccagcattttgggaggctgaggccaggggatttcttgagcccaggggttcgacaccagcctgggcaacatggtgaaaccccatctctacaaaaacatacaaaaattagacaggcatggtggcatgcacctgtagtaccagctacttgggaggctgaggttggaggatcacctgagccctggaagtggaggctttggaagtggaggctgtggtgagttgtgatcaagccactgcactccatcctcggagacaggaatgttttatatatataaatatagatattattttacatatatatattacatatatatataaaattcctgtgctccctagggtgattttatatatatatatatatatatatatatatatatatatataattctattctatatataacattctactgtagatatatattttaaaaaactgttagatattatgatattatgagttgcgacagtgatgatgatcatcatctcaactaggcaggtctctttttccctggcaaactattcacaggtaggctactcacaggcttatcctcctaagtagtgtgtttatatgtactcgaaataactacataggacacaatgtatatgctttagtccactgaggaaatataaagaaaaaggatattcttggtctcttgctctttctgttttttttttgttttgtttttgttttttttgtttttttttttaagagacagtcttacttcattgcccaggctggagtgcaatggctgttcacagacgcagttataactcctgggttcaggtggtcgtcccgcctcagcctccttagtagctggtgtgtgtcaccactctgctcagtctcttgccctttgtgttgtcattcaacatcgaaatacatttcttcttctttttttaattttattttgaaatggagtcttgctctgtcacccaagctggagtgcagtggtgcgatctcgattcactgcaacctcctcgtcctgagctcaagtgattctcctgcctcagcctccagagtacctgggactacaggcatgaaccaccatgcgtggctaatttttgttattttattttatttttttagtagagatggggtttcaccacgttggccagacttgtctcaagctcctaacctcaagtaatccacctgcctcggcctcccaaagtgctgggattacaggtgcaagccaccacagccggccaatttcttcttattgaaacgtagatgtcaacgttgcatttgttagatatttcttaactttctctcttatttgagtatctaacatattgtatttcttttgctctagtgtaatatagctaaattgctagactctgaattaatatcaataaacatttatatcacacaagatagtgttatactaaatatatttaaatgttttaaatggtttcaaagttaatattttgagtaatatgctatggatggaaagcttattcatgaccagtataagttatacaaaattaatgtttcaaaggatgtctgaatttcaaaaggactcttcaaatgatcagctccataattatatttttcatctaattttatgttcaagtgctcccctagagtggtgataagtgaaatcatcgattaatgctaggactctaagctgacctagaaaaactacatttgaagctgtattcaaagaatttcatttagccttactaggacaactgtcattcttatgtaacattttaaagttttgcattgagattgggcttgaaataaatgcctgcatttgctcagagactgttaaggagggagatagagtaggaagttgtctccagaaataaaatgataatgaaacctattgtttatcgagtacctactaattataaaccacagtgattatatatgacataatttttcatagatactaaatatatgtatgtatatttgcaatttttatccttgcaaaaatgacaagtataattggggtatttacattctctttttaaagacaggacaataaaatctagtaaggttaggattttgcccaaactcatatagaaattagcaaacatgagatccaaatccaggtctgtcagaccgcagggtctttgtttactctgctatatcatatttattattcattgatggatgtattaaacaaagattaattcagcccttcccattgccaggaactgttatagatgctgaaaatactgcagtgaaaaaagacaccaagttcttgctttcaaataatttatattctagtgaaggaaatagaatatacaaatatgtctagtggcattaagtgctacagagaaaagagcaaagcagtgtaacaggatcgaaagtgatggtacaagaggtatttgtactgtattgctgtggaggtcggtgatcagggaaggctccttctgtaaaactgacatttgagcacaggcctgaaggaagtaaaagagtgagcttatgggaatacatgaggaaacagcctacaagcaaaaggaggagctcatgcagtgctcctcaagtggggacttggtggcatgatcatggaacagaaatgaggctagtgtatctgaagcagagtgagtgttggggagagagtagagcacaaggtccaagaggtggtgtggtgtcaaaacctgtagaccttggtcaggacgttggcttttactctgagggagacgtcaagtcactggaaggctttgggcaaatgtctgacatagtgtgccttgtgttttagatggagaatagactgtaggacaatggggaaggatccaggagaccagttgggaggctgttgcagtagttcaggcatgagatgatggtggtggcttgagttgggttgtacttgtggaagtaatgaggagtggtagaattctggatctatttcagaggtagagccaaccagataagctgatggtggatgtggggtctgaggaaaaggaagggtcagtaatgactgtcagtttttgaccctaagtattatgatgagaagcttttcgggagggaagatgggaaaaaaaagagttcagtttttgtatgatgtggggtaggttttttttctcctctatatggcaaacgattcccagcacatttcttagcctttctcctttttcattcatcatctatttttttatttatcatctatcaggtgtccatttatgtccatgaaaataggtttaccaatcgtcttgttcaaagcttccatcttactgatctttatcacttgacctgtaaaatattttgttgttgtcattgaatctatcaactagcaagacaggtgtgttaaatttcccactgtgatggtggatttatcagtttatcttacaattctattgttgttatatacataattgaaattatgatactggatgcatgcactaattcttgcattaattctttctggtgaattcatgcattaattcttcctggtgaattaaattttcctcaatatgtaatgctctttatcaaatactctttgttttaaggtcctttttgttttttatgaatgtgtctacacacatgatttctttagatagcatttgtgtttctgtttttgagctcctattgtcttgaactttatttgtgggaatccctggggtctgaattcagattcacttttgtcagacacttggaaaccctatcatctggggttagtttaaagtttgatttaggacttgtttggcttcacagatagcatgaattttgatcccaaattgaatattgcgggcttgtggttaggaattgtcaggaacagtgttttcttcttttcctcctccctctgagccaaggtcaaaacaggcttgtatccccatcattgccctttgcaagatggggcttttctggtttatcactgagggtgcagcccttcagggaatccactgtgatgtgaccatcccacttttaatatctgaagttggactcctggttttgccttactaacacccaagtttgagttcagggcaaacacagcctgtgcttgctaatcctacagaattttcacgtcttttcctttttgttctctgagatattccttactttcctgccatccccaccaggcattaaaagatgcttcatttttgtataatatttttcatctgttgtaccaacaaaggtttctctgaacatctgattcacagtagttcttgctgacatgcaacgttgtaaacataggagagtttgaagccaaacctagcactgtggtatctgttcaccagctatgtggggtggatattttcaggttgcatgaagatcctgttcttcacaaccattcactcagtggttttagcctccactaatgattgttgcctgaattacttattacaccgaggctacaatatggtgattttcaaattctattatttcttttacatttattagttgcctttttttctgtaaagaaaagagtttttccccagcatctctttacttttttcttgtcttgtgttctcatggatcattttttaatttgacgtgttatacttctttactatgataattattttgatactcaaattgtcttgaattttagggtggaagcccctgaagccaactcctgtgcccttttgctgtgcttcactaaagtctttgagtctatctttgctttttggcagaataagatgtcccaggctggctttatactttgtctgctccagatctggaagcagccaatttttctgacgaatccggcttcttttgatgagaagcgacattcagaaaccaaaattggagttctggatgtacctgttcctacaagtgtcattgtatttgggcccttttagtggaaagaacaagaaaatctgtgtgtgtgtgtatgtatatgtgtgtgtgtttaaatcataagtctgtattggtattctcaactgagagtttattttctctgaaaaagtagtgtcagcaattatagcatagaggtaaacaatcccagtttttcgatgtacttttggctgtttgaccttggacaatttacttaatttcactgtctttcttagtttccgcatctccaaaatcagaataataaaagtacctcaagcatagttgtgagaaataagtgtgtggatttatataaagtgctcacagcagttcctggctcagaggatatacttttaaattatttgctaaaaatatgaggtcggaccgtgtgaaactgccactatttgactgtctttaacctacagaaataatttcatgcggttcatcctgataaatgtgaacaccttcattgaccgaatgccttttaggggcagcaccctttgctttaaggtccattttgtttgtatccacagctctgctgtggataaagataagtaaaagagtctctgccttgaagaagctgagagattaataatagagctataaataaagtttcctagcactgtagtgctttctgcctgtggtgggggaatgaagcaggactcttcttaggttaccacaagcgacttgggggaaggagacaaaatctggcagtctgctgaacagggcggaattttagattgaagaccctagaattaaggtgggaactccaatgggatgtcctcagggagtcacttaggaaaatgatgaaccacgtgtgtgcaataatgtgtgcaatttgacacacagttttaatgcagacaaaaatctttaataatcatgaagctatttccataatatgaagaaatttaatatatgttaaaattctatgtatttctttgttgttttccttttttagaattttctgtgtgtctttcttatgtgccggaaacccaacctcagagcagtattttcaccattgagatggtgccattaaacacttaaaatcggctgggcctgtgatcccaggactttgggaggccgaggtgggtggatcacgaggtcaggagatcgagaccatcctagctaacacggtgaaaccccgtctctactaaaaatacacaaaaattagccaggtgtagtggcgggcgcctgtggtcccagctactcgggaggctgaggcaggagaatggcgtgaacccgggaggcggagcttgtagtgatccgagatcgcgccactgcactccagcctgggcaacagagtgagactctgtctcaaaaaaataaaataaaataaaataaaataaaataaaataaaataaaataaaataaaaacacttaaaatcaagctaatgtgattatccttcacaatagcactgcgattagaagtttatatgatgatcatgttaagatgaacatggggattggggtgctaactttatatatatacacacacacttatatacatatacatatgtatatataaagatctctatctatctatctatctatctatctatctatatatatatatatcttttgactaccccacaacccaactactaatagcctactgttgaccagaaatcttactgataacataaactgttcgttaacacattttttggtgttacgtatattatgtactgtatgtaagctaaagaaaagaaaatattaagaaaatcataaggaagagcaagtagatttactgttcattcagtgaaaatgtattattgtaaaggtcttcatcttcataatcttcacgttgaataggctgaggaggaggagaaagaggaggggttggtcttgctgtctcggggatggcagagatgaaagaaaatccacaagtgagtggacccacacaatttaaactcatgttgttcaagggtccactgtagtttattccatgattctgtcatcatctactgttaaatattaatgtaatgagagtaacataaaattaacacaactatgtggctgaagatacctgatcttccttcagaagttagtttcttttactggccagtatttagtctctgtattaatcacaaatatttgtacaaaatgtattgttgttgttattattattattattattagagatggagtcttgccctgtcaccaggctggagtgcagtggcacaatctcagctcactgcaacctctgcctcctgggttcaagcaactcccctgcctcagcctcccgagttggtgggactacaggcgtgtgctaccatgcctggctaatttttcgtattttagtagagacagggtttcaccattttgggcaggcaaaatgtattataactttgtttcttgtaggcatgttgcattcttatactggctgtaattattttcagaagtgctttaattgatgatagtgtcagaatggaggaatattatgagactcttctcatgaagtgtcttttataacttttaatgagcgcaatagttagatggttaccacagctggtctgcttccctactgtattctatgtgtccagttattttcccagtccttatgaaattaaaagcaaagtttttgcaaagtactaagaaacatcttgctaaaggccaactctagccaggccttgacttttcgtatcatgtgtttcatattttacacactttgactgtaatgagactggcatatccatgtgagtcacgctgatgaacaggatgtgtgtttgtatttacgttctgtatgagggttttttggaatatgtgaattattataatcataagagtatattaaaccatcttgataagaaattttaaacatgtaaactattcattcaaaaaaacatttacagagaatttccttaaggaaaacctgatgaatggatgtttatcttttaaacattatatgattgtattaataacacatgatgaaaattctatccactgttatctttggctggtgagtttttcctttgtgtacattctgaatttccaggtttttcccttgatgcaccagtatgacttttatgattatcaagattttagtggattcccggatgccttttatttggtgaagaaaacaatgttctaatacaaagtgaatgaaaatagctgcacatatgatattaatagccagcatttatcaaggaattactgcacattaagcatgtgttaagtattctacatgtgttacttaatttaatattcacacaattgtataaagaagatgctatttttatttctattttatagggaaattggaacttggtgctgtttaataacatgtacaatatgagagctagtaagtatcggaactgggatttggacctaccttcagaactcaagctcttatctactgtgtcacacttgatttattctgatggttttcattgatgctttcatttatgtgacatgtttttgccagtttgcattttgaaggaggaagcgctgggcttcaggagcagaaggatgaatgtgacatagcccatgctctgaaggagcttgcagtctacttgattagacggtcatggagctgtgggcacatctgcctgggacgaagagagaggaagtatcaggttgacttcttggagagtgatatttaggcttagctggtatagaaagggagaagggtattccagatattcggggaagtgaggacagaatttgcaaagcagcagtttttgcaaatgactcagaaaccagattttgaaaagccttggatgcctagtgaaggagttctgggttacgcagtgtgaacactggggaaccattaaaagaatgtaagcaataatagggtgggatttccttatctgttttattttttgctctttttttttaaataaataaataaaccaaagatttctctagcaatggtgtagaagttctgttttagagtggagagtttccagttaggaagatcatattgaaggctgtctgtcaggagtccaagatcaaagtccttaggggataggaagagatgaaaaggtttaagtgcctgggatttcaatctaatactaaagttgtacaaaaattcataacacttttggcagtatattcattaataatcgtttctcaattgaatgtgtttggaacttattcagccagaagctttgatactctagaaacaaaattgtttatagcaaacaatccgttatacttgtgtttttagcctagctcttagcttctgcaatgaagttgagacttttaagaaaatatatacacagtacacacaaacacacacatgcttacacaacaacttactatttatcagcttacactaagtctcaaattggtataagttttaaaagttaagatcttaaatcttcatgaacagatgatagtgttattataatgtgacaataataaatttatttttattaaatgaaaatctttttgaaaagttcgtagaattttaaatgtttgctgtagtcaagaaatattatattagcaatatcaaccacagataaaagttttatattatttgcctaatccaggggtttgcagatttttttctttaacaagacagaaagtaaatatttcaggcttcacgggccatgcagtctcagttgcagctgcttaactctgctattgaaggatgaaaacatccacagccaatatgttaatgaatgaatgttactgtgttctaataaaactttatttccaaacgcaaatgacaggatggatttggtatatgggcagttatttgctctatagttttgtttgtttacttgttcttgattcttaattttataatgatgcaggcttacactgaacaatactgtatttggttctagtagatgcttcttttaaccatattggtattcagtgtattgtcggtgatgcttttccccttagtgacatacagtaattttatatttggtaggactagatacattaatttatttgttttggcctttatgtcttcttgtcatttggatttcatggtgacttgactgtggtttgttctttgtaattatttcagatattcacttaactagagaggttgttctgtctatcagaaggctgattggaggcatgtgctggagctagaccagtgttttgcagccagtggtatgaacctggagagaccaaagtttatagcctctctctccactgcctccaagatagtgactgtgactgggctcctttctttccttcgtttctgccttcctctatttctggggttagttgtagatattttgaatactgatacacagtaaaattttagtttagaaacacaaaattttgggggttcatttaattgatggaaaagagaatatttcactttatcctttaatacttggtttaggaataatatttgttctctgtaataaaaatcacaaatgtgacaatataacagtttctcctactttaaactcacttgattcatgccatctttccttattgcctcagcctgaagtggtatctcatgtgccatggttccgttagtactttgttcatatcactcctatgaaatttctgtggcactaaatttaatatgagattattagctcctggctggaggtgaaggtggaggtggagtggtgggtacaggggtcagacagggaagacttgcctcctttatcttcagccgttcctggagtctaatgcagtgcttcacatgcagaaggttcagatcagtatgtgatgatggaatgaatggatagatattccacttgaaggaacaagggtgatgagtatgatattattgctagcagtaatttccttcatataccttaaacacataaaacttcctagtgggtttttgtggccatcatttagctcacccttaactaagcatcaatcctgtcctaagtattattttagattctggggatacatactcaattacttaattatctagtggggaagacaaacattaacatattattttaataaaatgtggcaggtgcaactctgggagctgtgccggattctctggagtctagaggagggcaccccactctgtttcaggaagatggacagtcttttctgtctcttgcattcccagccaaatcctgaaatatgcatataggcaagctagggaaataacaggaagaagagcagaagaaaatttagactgaagggctggctttaacagaggtgggtgagacaatgaggtacagataaggattctcaagctgtgttgttggaggataagtctggagaaatacactggggtcagcttctggagggttatgtgtatcatggtaaggagacttgaactttatcttgagaggaagtagagcatacagtggtcaagtggccattgtagagttttacccttgcctctacattttctgaatgtcagacaccttctctgtttttctcacataaaatggggatacaaatgatctatctttcagggtttgtgtattcattcaacagtgaatttatgaatgcataccatgttccaggactctaggccttgggaatacagcactaaacgtgagataaatccttattccaaagagcttatatttctgagtgtggtggtgagatagataagaaataatatcagtgaaatatacagtctgttaaatggtgggtggaggataaagaatctgggactaggttaggagcgaggaagagggaaggctgcaattttaaataacattgtctggaaagaattatttgagatgacaattgaatgaagacctgaggacatggaggcccaattaattgagggtatttgagagaagagcgttttggagggaagagcaaatgcaaagaccccaaatttaatgcctgacatgtttgagaaatgcaaagagtcatgaaagttggggcagtgggagttatagtaagagatggggtcaggctagtagtgggggccagagtttgtaggtctttattggtcattattaggatttggagtgagataaagccactggagagttttgggcaagtaataacataatatgtttcataaggacaccttggctgctgttttgagaatagcctagctagggcaagggcacgaggaagaccggttacaaggccagtttttaggaatggagtggtgagaagtggtgcttgtaaggaattaaataacttgtaaatcacttcatatttaccgtgtgcctgccaaatggtaaggaattaataaatgtcagctagcgtttgactgttaaagcttctatctatgctcagcaaacagggtttaattcttttttaatctttgagtttcacttgtaagaataggaactttgctggctaagataataattgctatcatttagccaagagcattcatggtgagttagcattgtacagcctccttagcatgttttctcctaatcttctcactaacccaaggtggtagctctccttacaccaagatctatgctacatgtggtaccactggggaagttgaaggtattcaggaccacagaattggtaactggaaaccaggtctgtctttttcaactatgtccattagattgcatctgatattcaaaatttcaagtagattctatcaaagaataaaatatgtaaggaaagaagcatatgaaggacatgtctgttatatttctgccattattggtaactcttaagtaataatgattatctatgtgcttaatagatgtcattgtgtgtgtgtatttgttcttctaaatggtatgtttgatatgatcacagacgacattgtgccaaatgtaaccacttttcctggcggttttaatggtgttgcaagagtctgtgatgcttctggttccttttctgtccgatgtagacaatgcattcatattgcatgatgattagtacaaagtacaaacaaggagactagagaaaagagaggctaaatgaccaaagatacttaccgcggtaagtggcagagttcatttgagaattcaacagattggctccagagtccgtgttcttgctgagtagatcaccccaagggaaaactataggcatgtggtattagaagggaccttatagacagagctcttcattttgcatgtgaaaaactggggttctgataggtagaatctgtagccagtaactggtagagccagtactgtaatctgtgcctctcacttccattacctggcacagccttaatttgatcagctgtttttccagatgaaaagtgtattctccaagggaagaaacaaatactttctttattctcttaaagtttgtgagtggctgaatgttttaatgtgacgatttattaaaaactgaatctttaatttgaaaatcagatttgtatctttcagttgttttatatttaattatattgaaaaatcagcaaatttagattctctcatttgtttcatgttcatttatgatagctaacacttttaagaacctgatattgtagccatatttaaatagttatttctacaaatcaaatataaggaataaaatatgtcttatttataactcacataaatttaaatcatagaagaattgaacttgacagagatcctcagtaagaactttaaaaaaaataagtatcacatgtatttaatccatacttcccagtcattgcaacaaagcagggtatgatttatgtagaaggaaaacatttttttgttatttttgtagtggggcatttattgcacacacagtaagtgttataaccaaaacaaatatacaattgggacactgtgtttaaattatcttctgtcatgtactatataaacccacaatagtttgaaatgtatttaagccaccatctgggacaaagcaaaagctgttccaggatccagcacatgatgaatgtgaggcgagtttcattaggagcctcctagcacgtttctgcatcctgaatcgtttcttccacggtttgaaagaattccaacatccgtcttagctccctgaggttttgagtgtttttaatagatcatgaaattttttgtggcctcctaaagaactaaatcagtgaagttaaacattattgcatttagatttccttttaatctggagagataaaattcaagtttgtatcttttaaaagcttcagcagaaatgccccatctgttgggaaaaattcttactgggttttgacagtttgaaaggaataatttctcttttaaagatgactcaccttaattgagaacctattgccatgataacaggtagctatttaatcaagtacccattaagtgctatgggctggaggcagttcaagccttaatactaatctggcattctaatccagatttgactggctgtaaagtttatgtgagtgtttttctttcaggaaactgtttgttgccttatccctatataatgacaatagtacaagtagtgatagttactgaggccttaccatgtacaggcatggttctaagcactttctaaaaattatctcacttattcataagaacagtcttaagaagtagatacatttataattcccattttacccgtgaggaaattgaagatttgaaaggctgtagaacctgctggaggttacatggctagtgatatggtttgattgtgtcatgacccaaatcttatcttgaattgcagctccctcccataatccccgtgtgttgtgggagggacctgataggaggtgattgaatcatgggggtgggtttttcctgtgctgttctcatgatagaggataagtctcatgagatctgatggtttttataaagggcagttcccctgcacacgttctcttgcctgctgccatgtaagatgtgcctttgctcctcctttgccttccaccatgattgtgaggcctccccagccatgtggaactgtgagtcagttaaacttctttttctttatagattccccagtctcaggtgtttcttcatatttcttcattagtccatgaaaatggactaatacagctagtgagttctagccttctaatttaactttcatggcaatgcaggcattattcctgtttttacagatgatatagctaggatcagagaggttaaataactcacccaaatcacatatgtaataaatagaaaaaggagctgaatccatgacttctaattccaaattcagtaattaaaaaaaacttcaacttttattttagatttggggtacatgtgtaagtttgtgacatgggtatattgtttgatgcttaggtttggggtttgattgatccactcactcaggtagtaggcatagtactgtataattactttttcaatcattctctctccctccttcccctttctaatggctcccagtgtctgttgttaccatctttgtgtccatgagtacccagtgtgtagctcccacttgtaagtgacagtatttggttttactacttatgagatgcggtatttggttttctgttactgtgttaatttgcttaggattatggcctccagccgcatccatgttgctgcaaaggacatgattttgtcttttttatatctgcattgtatctcatggtatatgtgtaccacatttttttatccaaattcaataatttttaaaactaaaacatgttccttcattttcagcaattatatgtatttttatgcatagaaacttaaatacaactatgtaaaatattttaaatatgtttaatatataacttaaaaatttttcagagaggtatttttttgaattttttttatctaaattgaatcagagaggaagcaaggttgtgactgtggatatgacttttcataactaccaaactttctgtaggtggactagaatattcactttttaaaacaaaatatagcaaaaaatataacaaacacagaaaagcacttgaaatattgtgtttgaagagatttgtgtataattttgctatgctattgtccaatatgatacagtttcctttagtagttactttaaatatattttttattattaaaacaatataatattcttttataggttatgaaaaaaatccctaaatggtgaaattaaagatattgaagttgaggtttaatgcagaactgcacttgttttcagggaacaaatgtcaatatattaaagagtttaagtagtgtctgataatggttatatggtgatccagaagtacagtaatgattctcttaaaatattccattttaacttattaggcagttatggcttcttatatatgctataagcaaatgtggtggaaacatattttgttttcataaaaaatttctgttttaaagccttctagacattttcaaacaaatctttgttctctattctgattaaggcagtactattccaaaatatttaggagtggttttcattaatttgctattaatgttaccattctggcttctttcctcaataggagactaataaaaccatgtgccgcagtaagataatgtactaacatgccaacatgcctccttatttgaaaccagatctctttttctggtttgaaagttgtggatcttaaagttctctggcacaaggaaggcaagtgaggttagctatggagagaagattgcacaattaaaccccattgtaatgtacggtctgtctggcctagagcttcatcttattcttgtgattatgaaacagtattctgtcctgacgaagaaggtagtggggaaaagtttctgggatttgtaggagagtagaaaagaaagatatttgggaaggatatttttaaaaaagaagggtatctctatgatttgctgacaattaacaagtcttaaacagacaatcttctcttaatatgaggttttatgtgatgttgtgagggagcctggtattcacaccagtggcctcagttatcacacagcagcgaaagtgaggaggtcaattttacttatttggcaatttaacgtgaaagcactgggttgtgttttgataattcctgccatataggatggtcaataatttctacttgcaaaaggaaaggaagcctttttcttagcaaaatattggttggtgaacaggaggagatcttagtgccttgttatttcgcatctgtgtatttgtcatcttattctgacttgaaaactacttcactatctatatgattattttggattctgagtagttactcatttatctcccaccaatatgttattgcaacatgtgtcatctgttaccctccccacatcagctaatcatttgacatataattttaatataatatgctaggatacagatttagtttgaagtttaatatttttcttctagctttggtgagtgaattattaattgccattgtaaagaaaaatgtatcatataactgacatgagaaatgatcgtcaaatatttggtaatagttttttgaggacttacgaacttgaatatggcactaagatttggttaacagtatttccattgatttcaagacttcagtgactaaaagatatttggaggagtcttggggaaaactaaattgtgaaaaacgctgaaaagaaatttgtagcattgcagactgtagcagagttatatgatttagatgccttgtgtttagccaaatcaaaaatccaaaaatggatttctgaatactaaagtattaggataatttaacaacatgcctaccgttaatttcctaggaaacaacttcgtgtgttatgttttgaactttcctttgtattagatttctagaaggacttatattattcttgtttagtttccattttttagaatgtttcttttgatgatcctgtaactctcataagagctcagacttttcccctatacctatatttgaagttgtcttttgtctctctagtatttcaagcctaaacctaggtttcactattttcttcttgcttattccccacatacattctcttagtattaccattacccaggcttgtccattttatccctcagtctgttccaagcctgttcacacctttcctgttctactgccttcacatctgctactgccggcttgtgataatccctctctggaccatggtagttgcttctggcagtgtcccttccttcccctctttcctgtggcatctcttctttatacaatggaagtagtaatttttaaaatttaatttaatttttatttgtttatttatttatttttagacagggtctcatgctgttgcccaggctggattgcagtgatgcgatcacggctcactgcagcctctacctcccagacccaagcaatcctcccacctcagcctcccaagtagctgtgactacgggtgtatgccagcatgcctggctaattttttgtggagatgggagtctcccagtgttgcccaggctggtctcaaaaacctgggctcaagcactcctcttggttcagcctcccaaaatgctgagattacaggtctgagccactgcacccggcttaaattttagattagataacatctacctcacctccaaagcccttcagtagctagctccctgttggactctaaaaatcccaactccttagtgtggcctgcaaagcctgcatggtctgcttctcctggattgtaagctccacgaggacattcattatcattcaggaatgttcattaaaacacttgtgtccaacaccggcactgtgctgagtactgggacatagtaataaattaataaataagaatgagagaagaaaaagaaatggaaaacaggaacacctgtggactctagtcatggaacttcatctactggtagaatagatattatccacgtaattgcagaaagatctgttaaaagtacaacactgataaatgctgtgaagaataggttcatgattttatgaaagcaggtaacagtgaagtcttactgagatagtcatgactgaggtgatgagtgggagtacaggaggttggggtgtttgtgtgagtggggagaggagagaatatgcagggagggggttttctagaggaaaaatctgcatgttcaaagcctgtaggggcaggagcagactgtggctagaaggcaggagcattggcggttacctgctgttaaggatttctaataagctgaatgtggggaggggtgaggaaaagggaggaattagtaggcttttggttaagcacttgggtggatcttggcaaacattaacatttggagaggttcaagtcttggtggtacttgtgtgtgtgtgagagagaggcgcgtgcacacacacacacacacacacacacacagagataaagcaagagaatatgagatctaccttaggcaaatcacttaaccttttgaattgcagattcctatctcatttgaaaaacacacctaaaaatgcctaactcattatggtgaacgtcaaatggaattgtaaaaatacgttataaactgtgagttggtattggtatacatgtagatgattgtagttaactatttctcagatatttccttgtattcctcaatattggtttgcagccattcaagctggactatcattttgatattcttcctcaccatatagctttgctcaagactccaccctcaacaacaaagtaaatgttttaactggagctctaactctttaatagcttctttgcttccatcctctttttctgcttctaagaaatttaacttctcttcttagtttttttcttaccacccttctcattttttttttccattccttccatcttgggctatatttcactttcagaataggactttcttccatttccctgtagcactgagcctaagactatgcccttttatgtttcctttgcctttccttgttggggagaggggtttaggattaagtgtaggtgtatccacatctcctaagatcttttaagattatattttattttcattttcttcttagtaagcaggttctcttatttaaaacctctccccaaactcatgtcgataacattttattacttttttaaagatagaagttacatattggacttataaggtatgggagtgccagggtgtgggagagcttgctttgttaactttttggtttacaatgaaggagatgctcccttttattttggatagaaggacattgttcctcctcctgtggagtaggaaagtttgctccaagccaagcatgcctccttcctgaggactgaggaatagctatctgcctgtgtctattcttcacattgcctggagaaagttagaatagaggtggagattgagtatctagccacatgaagtgattcttactgtcattcatatttaacatttattgcctttattttggggaaaaatcatttagatggaagactcctctctttttaattttatctcccattccagctgatctacagtatattttctaggagaaaatagatttagatactaattaggataatttaatgaaatataaaatctgtgcgaggttagttgttgctattccaataggaaaatgaaactggattcagttatcttattgctaaaatatgctaaccaagattcagctattcagataactttagagctgtaaaatatattttataactctgttttcattctttcattaataaatacctgcagtaattccctggcttattttccttacacagttgttagagtaggactttgttttctgtcagtttcattcatttcattattgtatttaagctgactatagagaaatcatgttagcaaatgtatatgaattatttctccctttccatgaggatctttctgatgtcttattttttttctgagagaggctgtgcattgactttggttagaaatggtctgtcttttcaaaataacttctatgtaattaaaaatggctgaactctaatatgaagctgttatcttgaaagctatttctatatttaaaaaatcttattttaagttgtagaaagcttgtatgttttatgttagtatctgagtaaagctatcaatggaggtacatatgtggttaaagtgatgaaaatataactatggttaaatcattcatctgttgcaaattacttatgacttctgatacagattaaatgaaatgctaggaaaaaaatatagatgagaaaaatataagtagcaccgtatgtgccaatgtgtgactgtatgcttgtatatgttatacacaaaagttgttaacttctgcaaaactttgaccattttcagtttacattgttaattttcatttatttgatttagaaagaccatttaaatatgttcttaaacctagtgacaaaatgtgtggaaaaataatgagaaacatctcacagttaaatatttttgtttttagattagtcatgaatgtcttctttcctgtagtgtgaatggcagaaagttaactattcatggtgtaatttgtgtgaaaacatctatacttgcatatttatttttattattctattttatagaaagcattaattctccaacatggtaataaaacatgtttttccttcaggtacactggtgtaacttcccttctgcatgaaaatatttcagtttatcaaatcatacttttcaactaaaatttggttaacatagaaatgttacaaccataaaaataaagcgctagtattaaaaaaggttaattttgattttgatttttactgtctttattatctttaactgttaaaacatgtttagctcctaaaatatttaatagggtaatttaaattactgcatagcaccctccttttagtagctggtggtaggaactccaaaccaatggaaatttctagctgaaattggaatccatgaagaaccaaactcaggagccaccttgttttccttcctcatctaatatggatgtttaatttggggtggtgactgacttttcaatacaactttaagagaacattctatataaactgttgcgtctccccagctaccctcagagaaaaccacatgtctaagctgaggggaaattcacccgcatggaacggagccagtctcttggagaagaacatggttgatggaacccctgaacaataaagatctgtgattactgaagcggctcagaatttcatttttgaggccactaaatcactcttggcctgcacagctgtgtagtcaggggaggctgcagatattattcagggcagcaaccacagagtctgttttttatagaatattttcccagtgcttttggaaactcatcaagtgttaaaactcagtattttctgggaaagttgaagatttttctctccttttatggcaatttcaacctcaaggtgaaatatttctttaattatgtgtaaactttattttttaaatgagaaaattacattaaaattacaattttgtgggacattattaagcaagaggggaaaaatctaccgttatgtgaggttgggttgtcagatatttttaaattcagaaaatttgctgagaattctccttccaaaatggtaaaagaaaatgagtgcaagaaacgcgccactgatagaaaagtgggaagatactgaagcaatgttgggaacagaaatgatcagggtgactgaagcagagtcatagagccttgaaatagaagtaaaaatgtaaaccacggagacaaacaggaagcttgtggaagtgaatgatggtgagaaatgtgttctacagtaagatcatgaggtatatggattcttattttacattcattttaaaagcatttttccaactgctaagatttatattattggaaagatgttttgcatcttctaatagaaattcattttggaatagagtggcagccacagtgtgggaacttcagaaaagggggtgatgtaaaatcaacaggaacagagtgttttttgaagtgtcagcagcaaggtctggctaatgggagcaggctgtgacactaggcagcatgtactccagaaagcacttctgctttcccgtagggcatcccgcagaccctggctgggctcagcaccaccttctcattgatgtccctgtgtaggtgggtatacgatacgtatgctggtcaagaggctgtccctaattctcacttcagtaagctgaaaactccctaattctcttaaaagcctgaaaactttacgtttttctcagcaaatagttgatattagtgaaggcctatttagagatttctttgatattataaagtgtgctgatgatcatagaaaaaaaaataagctgcatttatttgttccctaaattcttaaatacaatcctcccaccgtgcataattacagtccccaaggtaaaaatctgacagttgtacttacataatagttacataatcagctacttaaataggttttagctattagctaaaataaataaatcctagagcagatttttctgggtagtgaagttgtctttttgtagctgttgagtactggagttctttttagataacctgatagtttagaaaattaatactgaagcaattaattgaaattgacttcaacctaccaattccacgactgtcgttttcttctttagttactttccattaattgactacgctacccctctttggccctgctgtacttgtacatggctatttattcatactgtgttttgcctctcattcatgccacaccaacagatgcataaagtgtccatctctctattttcctctgaaaacacgattttgatgcatctggcatggccttctttttccttgtgcatattagttgaaggtacgtgtgagctggagctagggctctcaccacttcctaactgccttgtgttggccgagaggtgctctggagccttccctgggtcctagtggcttactaggacctagtagtttcttcctttggcctgcatagaagcctagcttagcatagaagccttccttcggcctgcatagaagcaaagctttactcattttggaccattgaggcctcatgtgaattctttgtccattttggatcaaaaaaggaaatgacaaagagagacagaaatagaggttagagagacaagagtctacaccagatggtgttctcaccttcaaacagctgctcaaaactgtacagctccatatgttctcttggcttatcaaaaagtcagaaggctctgagacattaagaaagagatacttgtgggcagctcatttcaatagctcgtagcacaaacctgtaattaccacaggacaggtgtgtgcagatcttcatgatgctttgaagaatctcaagttcatttgagaacattgtcataatgacccatataaataaacagccaaagattaatttagctgccaggtaagcagtgaccaaattgagttttcagtaacaaacccagtcatcatttctcctgggtaagcactggaaccaacatacttatgattttattatgaattatttgtgtatgtaattaataataatgtatttctgctctctttgcttttatataggaactactcaactttaacacaattctaagttaatatttattcttgttatatcattatttggaaaatgtgcttagattaataagtgtaaaaataatcataaaataatacatctcaatgttctggagtaaaattgttattaagtgtatgaaataaaaaacaagttatgtcttctcttcatcgtgtcctttctattcccaaggtaataatcacctaatagttgggtatctgaatttctatatgatcattctcaatctcactctcaatcatctccccttttgtccatccatccagccccccgttcatctatccatccctagattcaccagttcccccatcctcccatgcccgcatccaggcatccacagattcacccatccctccatcccccggtgccccatccacccacccatccacccattccctcatccatccatccatcctccttccgttgatccatctacccacccatccatccaccctcttatacaagtgcagcctttatctcataatgaaacttgctttttaaatgtagttttgaagataaataatacatatttgtagaagtcacctgtaattgtttattgacctacaattcatggaaatgatgtcctagtgctttatttcttagcatgtagttatttagttgggactttttatttttgctgaagcatggcaatttgtgagcaatttaagtctctgccttgtggtaggggttggacccttctatgggtgtgactcttcttttttattgacttcgctgatgatagaacttaggggaaaattatgagaggaattaccacaagtgtagaatcggaattttaaatctgaaaaggcttggagattgttcagaatttgatgtaaacatttttctggcagtgaatgaactgttaaaactgtgatttgccggtggaggtaaacaaaaaccaaagactttttctttgcttgtcctaatttcagatcctagttcgcttggtgcctgtgtcttcatcttcgtatctattctgtcttcccatctgataaaacttgaggttggtttattctgtttccagatgctctttttattctgctacccagaaccctcttggttgaaaaagtaattttaagccattcttttttttttttttaatcatccagagtctcctgagctaatttagttccttcggcagctttgactgcccttttaaacatccagcatacctgagtgggttctgtgcctctatagatgttgctgacacatatcactcgtatttatttttgcctggtactctcatttgtatgtaagtgtgttcctaatgttgagtttccttgcaagtcctgaactgtgttttctactttcttcttccacttcctttgtactattcatggtttttacgcacgcactatattaatgcgatgatctgagcataaagtacgatttttctaaagctcatatgaatctgtggagttgtgtccaagcagtgaatctggttataggaggccacatttacagcatttctgcttaacagcctgtccagcaggcagctccgtgctgtctagtaagtcctcattctgatttgtatggtgtagatccatctgtgcagaataaaatttcatcccttctttccctctcccacctcttctatcagattttagacctcataaaacctctcttcactgctataagagaagagggaagaattcaaccccagggctttgctccaagctttgaggatattcacatggtgatagcttttaaaagatagagaatttcagaacaaggcataagccggaagttgtgactctgcatgcagaattagcagttagaatttagaggtacataagtatctgtaattgccaggaaactagatgagtaaaaatcttccctatggattgcgcctttttttcttacttcatagctctgtcagtttttactttgtgtattttgaggtcatgccacttatgcatacaagttcataattattacatcttggtggacttctctttgcttattggatagtgtccctttttttttttaccagtcttaatgattatttttaattaaacttttttatatcaacattgctgtatcagctttcttttggctagtattactttgatattctttctaaccccatgttctcgcttaaaaaaattgttttgcgtgattttgttttaagggtgtctcttctatgcagaatatgttttacttctgatttttttcttttcttttcttcttttttttttttgagatattgcttcactctgtcactcaggctggagcacagtggtacaatcagagctcactgcagcctcaatttcccagctcaagcgatcctcccatctcagccccccagagtagctgggactatcggtgtgtgccaccatgcctagctaatttttttgtatttttagtagagacagagttttgtagcccagctggtcttgaactcctgggctcaagcaatccacccacctcagccttccaaagtgttaggattacaggtgtgagccaccatgcccagcctacttcataattttaaaaatagctctgataatttctgtatttgaatagggagtttgttatttacatttattgtatttgtgtgtttgtatgtattcatattctattgagttttcgatgaccattccttttttttttttttgctcctttttttcccaacttcttgctttctattggattgttagggatttctgtttttcactttatttctctctgcttatttgaaagctatacagcatggttttctttctttagggatcactcttccactttactttttaaagatggataaattttatacatttaaaaaatttaatctgtatttatatcttcttcctgagtggaccttagcatgttataaatgctcactgaataattctcattgttaattagagtttggttttatttttttaaatgcaatgtacttacttattttagagtaaacagttaattaaatctatattttacttgtttttttttctcttttatcctgttgcattctcatggattcatttttctttttttgttattataaattcagggggtacatgtgaaggtttattatatagacatattgtgtaatgatgaggtctgggcttccagcatacccatcacccaaacagtgaacattgaacctaataggcaatttttcaatctttaccctcctttaaccctcccctctttttggatttcccatgtctattaatttcatctttatgttcatgtgtacccattgtttaggtcccacgtaaaagtaaaaacatgccgtatttgattttcggggttatttcacttaggataatgacctccagctccatctgtgttgctgcaggagacatgatttcactctcttttatggcctcctagtatcctgaactcatttttctacatactgaggtgtatcctcaagtaattgattcaacaatggtctaagggtgggtggctttcttttaattttagttttgaataatactttagctggacataacagtctagattacaaattgttttcttctcggcacttcaaagatgttatcctattgtcttccgcctaggacacaataaacaaaaatatttttgaatgaatgacacgttgttgctaataagaatctgctgtcagtctgtcggttggtcctttgttagtaatctgttttgtctccctagtagcttttgctacttacttaactttgtttatagtctattatgttgtatctaggtattgattcagctttatttgtcctgaatcctatgtaggacaaataaattcttgatgtcaagaattctaactttattaaaattctcaacttttatatctccaaatagggttgccttgtctttatgtgcagtccccttttctgcaatttttattagatagatgatggagcctctcagttgttctgtgctgtctgctcatttcagcagctttaaatttcctttctcatgttttaaatttataatgcttttatctcatgttctttttacatttctgccagttgttgtttgcagaagttttacttttgagggaggaagacaaggaactttcccccttactacctatttgaaaatcctaaacaaacttccataaaaataatttcatgtggagaaaaattcatgtttaaattggtcgatcttgttcactgtatttcttgggattagaatgtatttctgtgtgttttggaattttggtgtgctggcttgtatttgagttggaagattccagcaaaagtcttttctttcccccacagcatcttttcacattagatgaaattgagtatgagatgggaaaatgagcaccacacgagttcagtttcatggagtcatcctaggtgtgtcctctctactgtttgagctcccttaggctcatggtctcaggagccagattcttgcctgctctggacgtagaggcccgagggcccatggtctctcctctcttaccactttcccatttcctgcctaaagatgtttgccatatttaaaagatatcagttacagccctatgactttgtatattttcagattgtgtttatcattgctatggattttgagccagagggcttatgtcagtgatcaaatgtgtcatcttgtttggaagcaatttgactggaagttgaggctagacttggggaataactgctgagaaaaaatttaaaagggtaagcattgattattgatcaagtgactatatcatttaatattcacacccagatacctttgagagtgacagaagacattattagtagtgatataggattataagtgtaagccagagctattataggaatgtagaattgtgtagtcgttgtgctataaaaggactatcctctttaaatttttagaaaggaatatttatgattcaatatcacagttatttttattgatgtctgtatgctacctagagttacaagacagctttttctataagaaacacatgattattgtataaatataaaggccaagagtctttgaaaacatggagtatggagctgagaaagaaatgtgtgtatgagtaaatctgaaactgatgttagtcatgaagtttttcagcaatatgtaccattccctctttaaaatatttatagtattacatctcatagtagctgggcctaattttccaaaaaacagccagaggaagaatgtcagcagtaggagttaattttgaggtaaagttgatgggcacattgtatcataagactatgtcttaactactataacctactataattcctaataaatcttgtaaataagcatatatgagaaatttatagtgtttatgtcttgcaaatacagaatgaaccaatatatagaatttgaattacctctagtaaaagtaaaggttcatgttatgcaaacacatttcagactttctatgagttgcaaaacttacatgtgtaccacacctgcctacatgttttgtcactggtgagatgtgacaaaatgatctctacttaccctatctttgaaagcccatgattttagtgattgtttctaaatataaatattatcagcttctcaagacaaaattcattttattctctatatctatgtctatatcagtataaaatccgtgtgtgtgtgtgtgtgtgtgtttgtgtgtgtgtgtgaggatcaaaaactcatgatgaataacaggctttaaatttttttattcgttagcatatttccccaagccaacataattcttgtttttctctttttagaataagctcttgccatcatgttggtcagatagaagaagagccatctcatcttttgtttgttagagggtttcaattatttaataagagcaagtaattaagacattaagaattaaagtggctccaagaagagctctgagactcagaactgcagagtgttgcctaggtttctaagtttggatttcaacagaagtgtgaaaagtttctgctgctgcttccttcaaaaatttttaataagatgtagtattttgacatattaatctgaattgagactgaaaaattgcgtaaactttggttaatcagatcgattgtcaaatccagctatattgctgtttcattttctactaaaatttgaattattcatatcaccaaatagctcagcaaaaatgtttagtttttgcatatatgtaataaaaatagttgataatgaggttctaggggaagcaaggattttgggtgtgtgtacatatgtgtgagagaatgagagaaggaataggaatgtatcaggatagaatgcttctaaaacctcattttccagtaacaggtttgatatactactgtggtcaaaaataataaagaaactataatagctatttctgtattgttataaatgctgaagagtcctgctctcagattacaagatacgcctcaaggaagaagtagtacaaacacaaggttcttaatataggtccataagaagtggcatgatgtaatatctggatatctgggtggtcactcttatttctcacacaataagaatgaactttttgggattaaaactcactctccattaaatttttagaatttccatctccatgtgaagaagtttgagtattattgatttcttcttctttggaaaatcagcccttcagcttttcaaagacaacttgaaaattggacaccaattcatatataatgcatatatattctccactgctgtccaaacagtcattttgtagcttcatagtagaaaatatagttaaaattcttaaatgagggaaagcacatgaattattatatagtgcatctctttatatgaccaaaaattatgctgtattctagcaatctggttatatttttgaatagtatccataattacaatatttatttacaattccatttaagctcctgtaattttgacggaaatggaagaaatttagaatcttggaagatcttaaacttctggttttgttggttgtttgttttctggggttacttttggccaggaggcagagagaagtgttatatagtacttgattcagcaacattctattttcaagtatttattattttaaaagtttttttatgtattatgtacaatatataactgcaattcctattttggttttatgaattattttttctatttttataaagtaaatctataaagttattctattatgtgtctgaaatattgagaaagaggatccttgtaaaagtggaaagataggagaccaataccaggttgtttctctgatccaggctggttctcagcatagctcaggaccctcagtaaatgcctgttgagtggaaggttgcatgaacttcatcgagtatggattcttaaaaataataataaattaaaagaaagcttttcattgggaaattggtatttcaaaaaatcagattaataaatccagtcttttctttcaagttttgttagtttttcttgaaaatttatggagtatatttcagaagctattttttttttttttttttttttttgagacagagtctcgccctgtcccccagactggagtgcagtggcacaatctcggttcactgcaagctccacctcctgggttcacaccattttcctgtctcagcttcctgagtagctgggactacagtcgcccaccaccacgcctggctaattttgtgtatttttagtaaagacggggtttcaccgtgttagccaggatggtctcaatctcctgaccttgtgatccgcccgcttttacttgagccactgtgcccagcctcaaaaactattttgatagaggtggtgtaatcttctaaagccagctggcacatttataggatgccaggctagcccatcagatgaagtgtccattgtggcagccacagatccacatcagatgggaagagtgtgatcagtgctgcatgcatgtttaatcaggtaacaatgggagctgtacaggaagaggattattattccaagaagaaaggacctatatcatcttgtggaaggtaaacgtatccattgggagggaaaaataatatatacatagggagagagaactgaaatatcatcttattggtgtaaaccattttgttagtatcagcattattattgttgatattcacatctgtatgatccatctgatcaggatgggttagctgtcaagtcatcaagattgactttatgacatttttattatctgtcctttcaaaccttatggttctaaattttcactgtttgcatgcactttcatgtataaatctgcatctggaaagactgatatttctctaaggcaggggtcagcagatgttttttgtaaggggcaaggtagtaaatgttttggaccatagagccatggctgcagttacccagctctactgttgcaatgaaaaagcagccatccacgagatgcaaatgaaaggtgtagctgttagctttctgttggctcagctgcacaaataggagcatgaaacaggctgtagtttgccagccactccttgccacatcctcctctgtcattagctacccgtcttggggttcgttctgctgtatgctgtatctacaggttcctgtttgttcctgtttggtggtctgcctctttgcttcttatgcctcactgaccttcttgtaggagaagcttgtggggacttccatacatctttcaagttcagatttgggaagatgtgtgtcacctctgcccttctttcctgttcctcacatatggaggcagtgatgcctttcacccttctggccagagcagcctggttccagagccagccttgaccagagcttgacccatgctgggggaacttagctctggaagctggcagggtgtgggatgagtgactggtgccaggtaggtctggggagatagatagatagtttgataatcccaaatcccaaaactgaaatgctccaaaatctgaaactctttgagcatcaatatgactgtcaaaataaatgctctttggagcatttcagattttggatttgggatgctcaactggtataatgcaaatattccaaaatctgaaaaaaattggaaatctgaaacacttctggtcccaagccttttggataagggacaacctgtatatacagaaagagaactgaaataatgtgtctttttggtgtgtgttattggtgtggaccaatttattagttatcagcgttactattgttgacacccacatctgggcaaggctgtgtaactggcaaactggaccctccaccctctcgggttcacaggcctttcttcctctgcagggagtgggaccttgccggcttgcctggacacagggaccgattgttccccagagtcctggattcccttcctcatcccttcaccgtgactgaagtgctgctcttaatgggcagattgccatcaactgactctgttctttaagtaccggtgacgtcgtcatcttccatgtgctgcccaattacatgattccatctcttggccgttactaggctctcagaactggcctgacccaaacctctttgcactttcttgccaccttccatccctcctgcccctgtgccttgacctctcgctgacccttgttcctgagggctcttgccttcactgtcagaccagcatgagttttgccaaaatacagattcttttcagtggagtggtatataatttgatttttttgtttaaactcctacattttatagtcattggtagttaaattacaaataaatcttacactaaaatttattttattttatttttatttttttgagacagagtctcgctttttcacccaggctagagtgcagtggcatagtcttagctcactgcgacctctccctcccaggttcaagcagtcctcctgtgtcaccctcccgagtagctgagattacaggtgcccgccgccacgccagtctaattttttgagtagagacggggtttcagcatgttgtctaggctggtctcaaactcctgtcttcaagtgatctgtctgccttgacatcccaaagtgctgggattacaggtgtgagccacagcacctggtctttaaaatatttttttaaggaaccttatcatcagtgctcaaagatagtcaaaataaattaaagccatataaaaggaccagtagggcctcaaattggtcatggaacatagggagcaatttgaaattaacaaaataaaactaatgctaactggattgtttttccccgagtaacaaagacatattaggaggatttctgctcctaaattgtcttttaccatcctagagtcaaaatgcttgatccaattcagtaaatggaattgtacaagggattagtgatgctttaccttttatatcagaaaaattctcagaagtacacgtaagtggaatttattcttttattataaaatcagtctatagagactccttcctatgtttacggatgagagagaggaaatgatagatgcttaaaagattaggtaggaagccactgtgattgtccagatgatagcctgggcaggatagtataagcgaaatggaaaaaattcttagagactatagagtcgtcagtgtcactttcatatgggccaagttgataaacttttaataaagctcaggattatttacataaccagtgtaattctctgggataaaggtctgaataagttagagtctttgggaaagcaatcttagagcagaggctaagagcagtaccaagaagtcagactacctgatgttaaagcctagttctatacctaagtatgggaagattatttaacctttctgtgcttagttttctcatctttaaaatagttcagtgaagaatcaacaatatcatgcagttgattatgtgaggattaaatgaaacaatacaaataagacacttagaacacttaaaatatgttatcactattattattgttattatttggagtcacatacacatggttttaaatcactcctccaccatttgctagctgtgtggctgtcagaaaattaaataaactctctaagtacccttctctttattgtgggggttgattgcataggatggctttagggatttgttcagatcaatgtaagtaatcttctaacacgtagactatgctcaataaatggcagcactaattttcattttctaaagagaaaaatacttggtttaaataacctgattgattttaggtagagaataaaatatatgtagaagtgaaatctatctactatatgtcatttcttcaaaattaatttaagatttccttccactggcaaatatatttaaatgtactgactaattaaattcagtttagtattttgtatggatagacaaatagatacaaacaaacaaacaaaaaaaccagacaaatgtatatataaagggttgtctctccaaacccaatattgataaatttttaaatcttgaagatggatcattttgatgaaaacacaggtttgcagaagatactaggttgttatgggtaaatgtctggcttataaaaatggtactggatattcatgcaaaagctcccatctgatattaagttaaacaaatcgaaattaaattcatttcaacaaatagagggccacctgagtgcaaagaaattttcccactcagtgtgtttctgtgtatcttttttaatttgtctaaatctttttaaaacttttaagcagaagcctctgttagggtaatatgatccttcactttaaatctgaagttaaatttccttttatttgctccaactgtacatttctctggtgtcaacaacaattaattctagtaaaatatgatttgatgattcttaggtctgtttttatatcatccaaactttttaaagttgttaaatttttctgaacttattttttccaatgtgtattttcccagcataaattggatccacgttaatagtcttattatgtgctagcaaatccatgcccagtctagagtgcaatactcccagtccgttgagtaactactatgccatttttacaaatgctatagaggaataaagaagaaatgataatgtgtaacctctcatgaaagagatgaaattctggacggagaatttgaaatcaaatgacaatttttgttgcatagttttgttttgtttttgcagttttggtaaacctttgataccatgagtgtttactacgagtgaacaagtcacaagcctcagttaatacaaaaatctcagaaatagtcattattggttgaataattaacctagattatttctgtgagaaatgtggatgcccatgaatgatgttacattgcatatgtcttctagtaaaactgtttctagttcataccaataaaatgaaatataatggtgcttggtgtttctcaaaaggcctcatatttccccataaataacctgggtctaacatacttgacatctgctgttgtagtagtgtacaatatatactcacatataaattggcatggtacaaatcacataagaactaataagaattatttatagacgtgaatggtgacgtgatgtcaaataattctgaagtggtgtgtttgggatcaattctaaaggagaaggaacatgatttgtcagaaaaggaactcagattgcataggaatgaacaagagttgtctaagaaagatgattgaattgggtcagagccaggaacttgaccatttaatgaaacaatgtacagtgaggcaggagagcaggagagaaaaaagaaaaggaatggaaggtctcaagagagttgaaaagggtatctataagaacttactaattgtagcagctgactaaaagtggaacgagcttcctagcaaagttgtgatatccactgatagagataataaagtcaagagcgtctccttataggatgttgtttacggaattcatacattgggtagaagacaaactagatgctttctaagatatgttccacctctgaaatgctgtgattctcctatttgtattgctaattagccaatgaatatagtgtgtgaaagatatgacagcacaattgattgctctgtaattataaattaccccctctgctaattacaatgaagttttttgaagattttttaaatgaagcagtacacttctttatgggaagatcatgtcacattttctaactgattttgtaaatcccatttaaaaaatctcctctactattagaaatgacaaatatcaaaactacaattttatactgagatttatatatatatatatatatacacacacacacacacacacacacatacttttttgggggggataagaagcattaaaataaattaagtagaataattaaaagctgtatgtttagctggtaatttttggagtggcacagattttgttatacactgtggtattcaaaaaagcccagatttgtcccaagtaaataaagaaatgtcttttaaatgttttcacctctaaaagtctggtttggtggttttaatattttcttatgttgctatatcaggatatgtatcttgtgcaaagtgtggcgagaacagtatgtttacatcagtgaagtatgtggtaactagaagagcctaaaaattgtgatttatgttggctgttggcaatttttttttaaaagtacattgactttgtgagaatttttatgtaaataaacaaattctggttttcatttctaatgctatttgattattttgtataaccaaacgggtttttttttttttttttttttgcaccagaaccagaagtagtgtaatgattaatatatcccgcctcatacttattttaaagttgaaattttaggtttttaattcaaagaccccattgtattcttatcctataaaatgaatgggctctgtgcattaaaatttttaccaaatagatatggttttatgaaatgacaaaatgactaaaggatggctcaaaatctctaatggacttttaaaaatttgtgaaatatcagaaatcttgggaatgtatgactttggagaaggataatgcatagtagtgatcagtatcatttattatcagcagaatggcagaacccaataaataattcactttcttaaggacctactgatgcctagtgaatccctgtgtcatcatcagcattatttaaaagatggcacagaaggaaacatctgggagccaattcatcatttaattaaattatcctgtaatgaaatcttctgtaatgagattaattttaattattaatcaggagttgacatgggcacaaggcatcattaataataaatgaaaggctttgtctgttccatttctaagaaaggacctagtttactgcaacggtgctttttatacagccatgctgagtcctcacaatgtggccatccaagaagatggcttgatttttatttcagaacctagagagaggataattgaccaataccttcagatttggattccaaattgttgaagcaatttttgattttaaaaatatttcaaggatgtcaagaatagaaaatgctttttcagagtaatttgggggtaaattcctaattcagggacaaaaatgtcgccttttagaggccaaaaataggtaatgcagtcacattttggtatcagctataggtatgttattgtgctcttgtgtggatggtctgtcagttcctgacctcaccccagatctgccccagtggggtcactgcctccgaccagggttatatgggtaataaggagacggcaaatcagtggacttacttataattcagtgggaactcaacagtttcccggaagtactagctctctgcgtggtcaccaaacagaagccttcctggagactctggccatgtcgcccaattcttgtggcttctcctttcccttagttgcctgaatggaagatggccataaagaggaggcttgagtcttgttctttgaagccaaggaaatcagggttagggccaaagtaacaggaaagcagacagagggtctttaagaacaaaacaaaacataacctgtttgtttcatcacccttaattaaagttcttctttggatttctaattttatctgaactaaactctgaaatctttaacaaggcttctcccactccttcagctttatttcagaacacccctgctgtgcctccttttcccagtttctgtcacccctgtgccttctcctcggcctgcatattcattgtgctatactctctgtgatattattttggcaattggaaagtgccaagacttcttccactttgaggctttctttttttttgagaccagtcttgctctgtcgccagtgttgccaggctggagtgcagtggctttatctcagctaactgctacctccgcctcccgggttcaagctattcccctgcctcagcctcctgagtagctgggactacaggcatgtgccaccacgcccagctaaatttttttttttttttttttttttttgtattttagcagagacggggtttcaccatgttggccaggatggtctcaatctcctgacctcgtgatccgcccgcgttggcctcgcagagtgctgggattacaggcatgaaccaccgcgcccagcctgaggctttcttatttgcagttgtttagtcagttcagtttctgctgctttctcttggatcttttccagatgtgctcctaggcatcctttaggccctagcttagatgtcacttccactgagaagccttcccagattcccagtctaatttcttccagtttgttatattctctcatgacatccattgattctttcaatattgtgctaaattattttagacctaccatgtgccaaggcttttgctttccttcctataacttatcaaaatatataattatatagtcatcctctgtgattattgatttatctctcccactagactgtaagccttgagaaggcaaggatcatgctgaaaccttggcaccatgcctggtacatagtattccttaatgaatacttgataagtgaatgaaaacaacagagatgagaacgtaacaatttaatctgtcagacacttgaaggagcccccttaagaggcaatgcattttccatgcatggaaatgtttaaaaagagactagacaactctgtgttagatttgcctccagagagaggactgacacatgagaaaggaaattgaaatagatgaccataacagtttactctaattctgtgtggtggtggttccaacagctaggaaataatcaatcaatagttgcctattaattgactaagacattatatctacttggtgacagtctctcaatatttgctttcttgaagaaattgaatattttttctaatttattctttaagcatttattcagagtactgtgaaaactgagacaactggttcaatattttagccacagcaatgtctaacaccaaagaatccctgggattctttttttccttttattttataccaattatgttagcaagaacacttaatatattataatagtgatgaatgtatagaaaagggaaaataagtaacatgatcattgtagtgagtaatatctgattaggttgtactgtaaattttgaatgttactactatgtaaagacaaactttttttcagatagctaagctaatcttgccttctgaaaaaaggtttttttagggtaaggaactttactggtgggctctatgtatactaaggaaatctttcttttaattggtaaaattccactgattggtaaaattccactgctctcagctatgtttcaatcttgaccacttagtgtaattaaaaagacagagcagaacaacattgagcctccttatagttctccagcccagtttgccattcagtccgtgaaaatgagtcagtcttggggaggatatttcccacatggcttaataactttaaacttacaagacaacagaaattcgaattagcagatgcatatgctaaaagaaaaacatcttttttaaaaaatccgattttaaagactaaattgtctgtgatgtgcctcaaaggcacatagggacagaattcgtgtaactcctcacattcttgtgcctgagagaagagagtcaaaccactagtgttttggaaccagagatttaaaacagcctagtccagaatggaacttttctttcaatgtgatttgttcatttgcaggttagtggtaatagtaagactgtcacagtaggatttttttgttgttgttcttgagggatatctggaaattgtattagtaattgtgattagcaggctctacctgaatgtattttcctgctaccatgtgtctagttggcatgtgtcaccagggctgaacattttagcacaaactcgaagttgtattgtgtttcagtagttttcctacatgagtctgcatttgccaatctacctatttgatctctcttagcaaggaaaaaaagaaccctagtgacctgcaccaaattctttataaaatagactattgaaaataagcctaatgaaagcttattttattaaggctgtattatgtactctgaagatcttggtaagtattagtataaatattagaaataatacctatataaagaatattttatgatccagttgtctttggttgaaaatttgcttttactgctgaaatttaaaaatcaagccagctacatatagctacaaatagctacatatagctctagctaataaataatactctttagctagttttaggaatgagaagtcatcaaagtgctaattgaccatttgatatagcttttgctatggtacaatgtgtttgttaggtgagagaattcttattccacaaaagaaaggataattatatttagctatggctgatgacctggaagaggcctttttgcatttccttttttcaagatagtttattgatctggatcactaaaagaaaattaaacatctgaaaaacaaaaaactggaagaactgcagtagtggttcagcaatgcacctgtgtatacctagggctttgcttctcctttttggctaattacagaaggactcaacaataagtgctcaagactttgattgctagtttatacatctttatatacactttcattctatctttaaattgtcctaaacataagtgaaaataattagtaacattattcattgggttcatttaatttgttcaacggatatttattgaggatcctactaaatgccaggcactgttcctgatgaacaaatgacagactctgttcttgccctcatgttgcttgcaatcaagttgttaaaagagggataaagcaaataaccatgcaaataagtatacaatccaactgtcttaagtgctgcagagttaggtaagtagttaaatggtttctctgaggaagggattgattgaagactaaaactaataaataggaattaagtaggaagaggttcttggagaggacattctaggtgggggaaataatgtgcacactctgtagcgggagggagcatggtgcattgtgaatggaaccccgaaaacaaagtgaagcatttgggtgcagtaaagctgtgcgggtaagtaggggccaggacatcaagcacttgtaggccacatttagggtcttttttgtctttgtccctagagcaataggaaagctttaacaagttagagagacagtgagtgagcattgcttttagcaaaggtgactttggctgtacttgtagaccataggttagaggcattaataaaggataaaacataatgattgtgatttattataacacaaatggaagaaattaaaattcatgaatccaaaatgatgcttgaggattgggtagggtagaggaaagctctgctttacagaaaaatgccaactattataaatagaaagaatgatggggtgtcaccatttttgcagttcccaataggataactgatcagggtgtcattaacagatgctaaaattgttagaggaatgtttttggggaataatatagtcctgtggtgccaaaatcctgctctaaagactgtttactaataagagaggaaaaaatgtgactttacagtgataatatatcagagtcaccactttaatcaagggattagatttaacttcacaattagtggtacagcctggtatcacagattgtgtgatgtgatagaatatggagtacctaaccttacaaaggaagtgtttctacaaaagaagaaaaaagtcaaacctgaccacaatcaagcctctaaacagaactctgcgtctgtagagaatgtaagaaatggaggatgaattaaattataccataaggtgagacattctataagaatcctagagtggatatttcagacaagattaaagacatgcaacagccaacaaccaaacaaacaattgtattttggttttagaaagctatcaaagaccttcttagtacaattggagatattagaggattttatggtattgttaatttttataggtttgataggagatcatgtatgtttactggtgaaatctgacatatgcaacttatttccaaacgatttaaaagcaatgatacatataaggtacagataagatacatatggaaacatgttaagaactgtggaattttggaggacaatatctaagtgttcattgtactaatctttcaatttttttgcatctatgaaaatttttacaataaaaagttggagagagagagtagatggcaggctgggtagttacttgcttcaccctatgcatctggccgaaaacagtttctggtccttggctaactacagctgttgggtaaatctttcaccaactcagaaagtcaccccaacatctaaaatcccaatctgcgaaattctccaggttctgaatacatgctgtgctgacatcggcagcgtaaatgtgtgttgtgtggtctgagtctcagtttgtggatacctccagcgggaacctggggcttaaagttgaatagtgtctgtagcagagagagtctttgtgcttgatttaaactacacagaatggatggctgtggaacaccagtgaggaggctatgcggtagacctgatagcagaaaatgtgatagcttcataccaggatgctaggatgagagagagaagcacaagatatgagaaatgaataaattttaggtggtaaaaaatgactggacttcatattatattgttaataggggaataagacagagagggatttaatgcatgactcgctggttttttatttgtataaatggaagaatgttggtgtcagacacagaggtcagaagtctgaaagtgggcaagtttgggaagaaagatccaagatttggtattagacatcatgggttttgcaatcaaaatggaaatgtgtcatttgtatatattgggtagttaaacacagcttaaatggaaaaacagaccttcagaaagtactgtagtacattaattttgcacaaagtagcactaatttgatacacatctctaattgctctcacttaaagcagtttcaagacaaataataaactctttttaattgcaacgttgtaaaaaacacttttctatgctgtttattctctaggaaactatgctatataaaaatattatataagctattccttttccagaatttgttcagatatccacaaactgagactcagaccacacacatttacgctgccgatgtcagcacagcatgtattcagaacctggagaatttcgcagattggggttttagatgttggggtgactttctgagttggtgaaagatttacccaacagctgtagttagccaaggaccagaaactctgttttcggccagatgcatagggtgaagcaagtaactacccagcctgccatctactctctctctccaactttttattgtaaaaagaacattcattctttgtaggagttagaaaaagctaaaaacttactttagagcttactttgtgggcttaccatctagtcattagcttaaaaattaccaaaatatttctaccgcaagtaatagatacccattttagaaacattgaaaatttttaatgagtaaatagaaaaaaaggtcactcaatttaatgagtaaatagaaaaaaaggtcactacatcaattggcatttgatgtagatagtttgtactcattttaaacttaaaatctgtttgtgccttacatgtagtgaatgaaataattactttttgtactatgtgtattttactatagttcttcaatgagaggtaactagatggtttatgtcacttcacttgaatgtgtatttatattccaggatggctctgtcaggaagttttctacttatttatagtgataacaattcatacaaattaagccttttatttttacctctaggagttagatatcataatcattcagcctctattaaatctatagtcacagaaaaatgatataaaaccatgaatcctgctacaacactttggatgtgttgtacttttacccactgtcacaaataatttaaaataatttctaaggaaacatcatggcagccccagttggtggtgcttagtgtttaactacagcatgatgactacagaagaggtaacaattttagttcttgctaaaactaaacaatattacatggatgcacatacacataatacaattcagaaaattatcaccaattgcttcttttttttttttgtcttctcagaaatgggcagatggattatgtactaaaaatataaacccttaagaatcaggtttatcctgtaaagaaggtggtgtctctgggcatcacttggctcttttgtaaattgattgtcacttcatttttatcgttatcagtaaaccttattaggtgctgaaatcagtgctcggggtgcagggtcaggtaaggaaagcatgtgctgtgtatcaggagacctggccacaaacttgtcatatgtctctacataagttattattgatgaaatcattgtttggtgagagtttggatagcctgtatagggaactagctaagtagttgggaaaaggaactgaagcaggacaaggtaataaagtgcatcaagatagaccccaggcccatttggttagcatctctgaaggagacaagttcaaataccaagctcaaagagctggtagaatgagtggagctgtatattcaaagtacctaagcagttgctatgaataccagtgtgaggttcactgtttgcctttaaagaccagcaagtgtgtaatggttgcagttaggtcagactgtatggatagaggtagtacagagccattagtggaagaatattaaatttgtttgtttttagaattggggaaatatgataacatgtgtctgtaaagcagaggttgtccctggagaggagaaggaaatatgcaagagaaatggggggaataagttatacttgcttattggaaaaattaggctctgaaaggatgaccatggattcttggctgcctaatgaaacacgacagccagaaagggcctgtggtaagtggtcagggcagaatgctcaatagaattgtgatttttgaggtgataagaagagaaaacattgtatttagagagggactattaagagttctaacttggttgaactcttggttgattaataacaagttaggacaagtccaggtaggtgtttataggtagccaaagtggataaaaactgggaggacgctgactttgaggcagcgaaggactgtgcttacaaggcagaaatgcttagtaacatgatgtcaaatcatcagggacggtggccaaggatggattacagaggtgcataaagtagttgctaaaaacaacaagtaggtttggaagagtgctcctgggcttgatagaatttagcaaccagtaggggtagaggttagtactaatataagtggtgaaaagacttaaaagagaaggggaagttacgtcacactatcagaagtaatcatatccattacaggggttaaaacgaaattgtctctaatgttaagactatatataggcttaatgtgttaaaacatgggcaaaagtcattccatgctttttctgtgtactagagggactgtaggtatagacacagaaaaaccatttaaaagtgaagcaacggatggtataatcctgtgtcctgttctttgccaaattgattttggactatccctctgaatgcaagaatggttcttctgctgcctttgcctcttttcacatctacataaagtagtgcttacatttgtttggagtcaaaaaataataaataaatgaacaaaatatatggcatgtcagatgatgataagtgctacaagaaagacgcagctgaaaaggaggtgagagagaattaaattagggtaggtgagccccattgataaggtggtgtttatacagagacttcatagacatgagaaaaggaagtgtatgtgtatctgggaagtgagcattccagggaaggatgtgcactggccccaaggcagaactgtgcttgagaacagaaaagaagccaagaggcggcagaggagggaaagtgaggagagggactcgaagctgaggtcagagggagtggtgaagtttttgatatcaagccctgtgggccaataaagaccttgccttttgctgtgagtcatatggaaagccactggaggataagatcagaagagtggactgctgatctaacttttcgaaagagtccctctggctgctgtgtgaagaaaggaatcatatccaggctgctcagcctgtgccactgagcagcctgaaattagaaggggtgggaaggagcagttaaagtatggatgtattctgaagatttagccaacacggcttgctaatggactggattttaaatagaagttatttatcatggagcgcaaaacttagaaaatgctaaggtctagcttctatgtttatacttgctatggcatagaccctggacatgaaccctgggtttgaagacccagaacatgcattttaagagaagatcttccaaccatccaaggaagattctgcaaattgaggaactgagtaaacacagcaaacagttgcctaggctacctgagggaatagttcacaggttaaagtagaaagaatagttgctttgatcaaaatgtggcatgagaataaatcctggcttttccacttacctttaaaatttaaattaccatataggttaaattcattcacctgtccatcaaatcatttatccattccactgacatttagtggatacctaccatgttccaaacactttgtaggtacgcaggaataaagcatgaacaaaacagatgaaaataatctgtgaactattctgagagatgtggataaaacattaacagtggagccctttatgaaatccactataacattaaattttaaattccagctggatttagctttatggacttccaaataagaatagcctaataaattctagtttaaaggtgtaacattgaacagaatttagaaaattcttttctaatgttgaaacaaaggacttttgcaatagttgcattaaaaaaaaaacattgactcacaaatgaatagctagtggagggcaggatttctagataagcaggacaaacaatctccactgtttctcaccaactatggagctaatggggacggtggctttaagccaactaccacttaaggggaaaaaaaaacttaccattcaacaagtgaaatagttagtgtggaatggatagcatctttgaaatttgattttcagcagtctccccaagtgactgaatacattttttcaaactatttacttttgtatcatgtcttctcatggtcaaattcaggatgaattttatgacaggatttggtctaatgctgctgaattgtctcataaatatcttgacaagtcatttgaaacatgctgggcctctaaagaggctgactgacatgaaagcactaacttggaatcatcagggcttaagggtaacttatggaccctaaggcagagccccaaatcaagatcataaaattacagacagtgagttgaaattgagaccagtctagattggtaccagcttctttgggtttcttcggtagcagggtggagtcaggtggctggtgccatggaagcattttggatctggtacctactggctacaattcattgttagattcaaggatgtgtccaggctttgggaagcccttctggggactggaaagggagggtccagtacccagaagggcttcccaaagcctgggcacatattcagagtccagccaagtgggctgaatttcaggacaattttctgggtccatccaagggaaggaagcacagattcagatcggaaggtgtagcaagggacaggaaagcctgttttatagtgagctctgagctttggtcattcattaccaggattacctgataacatggtatattgctgccatccagtttaaggtacacactgccatatatatatatatatatatatatatatataattctttatatttctctgggcttaactgaacttagcttaaggcacatgaatttatcattcatacaataattatttagcatttttatatctgggcctggctaggattaacaggtattgacaaagaggatttgagatttctgaaatctttatttagcataacctaaatcaacaactaggattgatagaaattaaaaagcactgatttattttaaaagtgaaaatttctttatattttgatgcaatattttaaaaatatggtacagatttgattcctaggtatttactaatagttctgcattttgaacaccattatctatattaggcatattgaccttcatgatttttgtacaattaatttttgcttaatttattttctaatttatagagacgtgaacagttgccataaccatgaacatttacagatcaatggctattggagttatttaatttatacatggctcagaaggtggcaaccatttctttcaaatgttatatggacagttctagaatgtatgcagatatcctggggaaatggaggggtgggtttctttacagaaaagaatgacctgagatcaacctaacaaaagcttggttgtgaagaaatctatcagaacaaaattgtacatatgaaaagatataattcttgcatgtgactcctcaactgtatttaccaggatgaccctgtgacctttcttttactttagattgattgttgatttatttcttactcatttgttcacacttttactctgtccccaccctgtgccatccttgagctcaatgctttctcttgctccactttggcttggcacagaccttgatctgatgcttgaactcagcttcacctgctatcctggatcactgttgttgtttttcattcctcggtgcttcccgagttccctagcagatagcctccccctagctcaacaataccaccaccaccaccagcacccccgccaccaccccccccacccaccccaccacccttgaccctctccatacacactctggtagactgtttttcggcgtccttaccttgataagcttaacaaagactaataataacaaataacacaccacattactatttacaaagtgtttacatattcattttcctgtttgagatagtggttcctgtttcaaaatagaaaaggaagagtgagatatgggttaaaggaggactatgtttttcataaatatttacacatatttacatatgtaaaagtccacataagtgtttataaatatgtttcagtgataggatggaaggtcaaatatatatggactcttatgatatatacaaagttttatttgtttatcagaaaaatactgctctgcaaatcttctaatttgatagccatttatcaataaattgtattcattattgaggaaatcattactttggtgatgtttgtcatatctgatttaaaagcaacctcttactcttgagaagcgagaacaggagagtgttcattcagtatgagtgaagatggagacagcaacccagagcatttataggagaaaggctggacatgttttaaacactggataaatgcaaggcacggttatatgccaagtcgcattggactggaagagatcgggagacccaagcctttgtctacatgacttgtctgacttatctcacatcttccatttctacatttaaaataggtgaactagatgtaggaccctgttatttcaaatatatgtgacaagagatatctatgcccctttccttttgaaaatatttctggtgtctcttgatatttgaaaaataaaattttacgaatttaagagtccaaaactaaatttcactgttatgagaattttgcttttgcttgtcaccataataacttgttatttattttaatcttatctggttggtcaaaacttcaggtcagctttacgatatatttgatatggtccatttatacttggaactgatccattgtaggtaataaaccaattaaggcagaaaactacccttagacaaagttggtatttttaaatgagctggcatttgaaatgattattttgatgttaatattatatatattttatacattatgcatttagagattatatttatgaaacatttcaatataacattgcatccaaatttgcatatagtatacataacccaaattatggattttgatacatattcttttaatcataccaaagttgcatcttttcatttactcattccatcttcagttgtttataacaatgtgaatggtatgaaattatgtgtttatataataattactccatatatcaatatcatcagaactgagaaacttattagcttatcttctagaaatgccattaatgccttgagattttcttctctctacttaatagggttaaatagatgccttccattttagaaaggaacagtcttattatttcttatttctgagaaaggactagatactccttgagatgtctgtaataaggtaaaattgttaactttcaacatgctgttttcctaactttgtgtatgtatgggacccatgatcttatctagccttttgaaatgaatcactggaattaagaaatttaaaagaaaaaaaatactgtgtaaaactatgctatgaaagaaaaattattaaatatgctcttttcatatagtttgctaattaaatactgcagctgttatgtggagggaaagttgcagatatgatgagatattgagtttgtcatttctgaagagttacatttggctaatctgtcttaacaaactagaattttccatacttttatgaggagtatgcgtagtaagtatgatgtatatatctttgcatagacatatgtacacaaacaagatttactaaaatattttaaattaaaaatgtaaatctaagacctgcatcctcaacctttaatttccaaagaaatataaattttgctgattatcaatctcattcaccttttctttgtaaataaaccagaaaacagcatatttagagagtacattcaacagcattactatttcatcacctgtttcagtaataaagtgacagaatgcacctttaaaataatactttggttgtatcttaaaaattaaaattgagagtaatgataattaaattgttaatctgtctgttaacttagtcaactataagaaaactgattttttagaaaggggtaaagtaataaaaatagtctattcattagattctcccttaattttaaaggaaaagtctcttataatacattttgaggcatattttcctattttcttctgcacactgctatgcagtgcttaagagaaggtaaggacatcttttagagaaactgagtatttctagttcagagttatttgaatgctggttacttggagtggaagacttgatcgtgggcagactagcaattaaagatcatgaaagtctacactccaggaggcgatagacacttgacttgctcactcctttctcagtcaaatgccctccctatatgccatctgtgggctgtgtcaggcttagggatcctgaaggaaaactacttttggagaaataaatgactgcttgatatgattgaagtgattggtttatacgttagtctttttgagttatgtcatctgtatgttccagaagtattaaagtttgctttccttcttttaaattctagatctaaataaatttaattttggtcagatttggggctaaaatttgatatagtcattcagttttttttccccaagggataattacataatctttcttgacgtggacttagaatgtacagtttcacaggcttgaaagactagtagtcagacgaagtcgtattctgatgaacaaaccttcttaagcatgttctgcaatgtccagatagaattaataccaatccaaattcatttttctcagaaagtttattatctttaaaaatcttatcattagcaaccttgggatctcaaaattatagtcttagaacattctacaagtcttttatttgtgcagttccttaattactactatttctacaaaataactgctaatatttattgagtgcctcctgtgtcacttcatactaagtcctcctcgtaatttatttccttctgtcttcctatcactttcatctggcaggtatgctcattttcctgttctcgggcaataatttgccccaggtggcttacctggtgagtagcagaatgaggatttcatgtcctgttgcctggctgtaggctttgagtcttaaccacacaccgtaaggcacccagagtgtgaactttccatatgatgcagaccagctttctgtttctgcaattctgcattttatggaaatggaagcattgttttaaggatgatgtgtgaccaaattacattttaaaaatctttggtaatactgacattgtaaagatgaagatccagtccctgctctgtggagctccaggtttctggagagagagtttatcagataattgcgttgtgttatttcaacggtcaaaaaagaacatggcaattctgtcatggggagggctttggagaagaccagatgcctgagatcaagggagagcaaaatacttcatttttgtgtaacattctcccgtgcaactttgttccacatttcatgatatccttttcatatctaaattcacatggacttctgattgtgctgtgtattccaaaccatgcatttccccacagtttcattgtgtttgctttcatatgcttttccatttctctttactactatgtaaaacatgtattttactttattaggttcttgaggaagggtaattatcctttttaaatttttttttcaaaatatataactgcatttaactcttctttttctgacttctgagatttaaaatgtgttaattatacacttaaaatggagatttacagtggtaatgtacatttaaaatggtgattattcatgcctccctgtgtcctccccttttgttttgtagctgtacttaataggaaatacggaaaagggcaagaggattgcccagatttaacacagtgtctagtaacaattattgctaacccctattatgcattattctatgccaaacactgttgccagcatttttcatggattattttatttactcactttaactttatgcatagataactgttgttatgcctattttagagatgataaaaccaaggcacagagtggttaaataacttgtcccaaaatcatgtacctagttagtggcagagtgaaaacatgaactgaagaagtctgattctagatactgcatgtttcaaacttaattggggcttctcagcttggaggcaaatctgtaatttatgaaccggtcattcataaatattcagtaagcatttcctggctcacattagagaattatttttccgtatgatatggtggtgtttaaatgttataaaggggagcatattggataggtcaaagagtaggtcagaagttaggtgtacgcttattgaaatctgggttctgccataattagttgtgtgactttttaaaaatcagttccttccatccttcaagctctcagcttcttcgtttgggtgatgattaagtatcactccttcaacagtgagtaatgcaaagttttctacccattcttgcttaaacataaattttgaagccttcagacccatgtggtgggcactgttccatctccattacaaatcttcctcctcatctcttcaagtagtttttttttttttttttttttttttttttggagacagagtctcgctctgttgcccaggctggagtgcagtggtgcagtctcagctcactgcaacctccacctcccgggttcaagcagtcctcttgcttcagcctcctgagtagctggcatgcgccaccatgcccggctaatttttttgtattgttagcagagacggggtttcaccatatcggccaggctggtctcaaactcctaaccttgtgaatcacctgcctcggcctcccaaagtgctgggattacaggcatgagccaccgtacccagcctcaagtttttaatgttggatactcctttggtgatctcatccagtctctgatctttaaataccattttttatagggaagattcacacttttatctccaacctagacctcttccctgatctgtagacatctaatccattggcaatcctgttggctctacccttaaaatataaccagaatttaataaatttctacatctttattgctaccaacctagctcagaggttcatctccatctcggtgattgcagtagccttctaacttgtctccttatggtatattctctgcacaacacccaaagtaagccattaaaagattaagtcagagcctgttgttcctctgctcaaacctggcttccaatctcactcaaagtcacaatctttacagtggcctttgaggctctacacaaacaggcccttagttgcacctttgacattgtctgttatctgtctcctcctcagttaattcaccctagccaaaatagcaggatgtggtctttgaatgtgccagacgtatgcacctgcctcagggcttttgcacttactttcctctatgtctaaaacgttcatctcttaagtgtttgtataactcactccctcagccccttcgggtgttttccaaaatatcttttgaataaagctttccctgactacccattttataatagcattcattcctcaactgctggacactccttacttgctttactttgcccaatagtaatgaacattctctaggtagcataataaattttagtttttttgttttgttactcttttctgtctccatcaattagtatataagtgttatgagggtatagattttgtagatttttatttcagtgttgtatacttaaccacctaggacagtccttgacacatattatgcacttaataaacatttgttgaattcatgtatgaataaatgaatgaaagaatggcatttgagttttattaggaacttttatcttcttttgaaaatgatggcaagttagttttggtctcttattctgatagctggggcctatgagtgaatgtatgctgaaacaaaatgcctcatgatccaggccagaaatgtaagtgtatgaaatgctttgagtgcaaataatataggaatgtgggggctgtaataaaccacagggttaacccctaatcagtgggctacatctcattgctatcataggcaaacagtgccttgccatttgacttagaagaagcagctgcaaatccagatttttatgagagctccagcaattcataaatatcgatttccaattcaaaattttgtgtgggtaaaataaaacatcttctagcaagattcaacttgtggactgccaatttgagagctctagtaaaagaagtataaacttcagacatctatctattttagcaaactttacctggtttaaaatgtcgtcagaaaagtttgtaaaataacttgagtagaaaatgtaggacatgaaataccaatcaaatcaagaatgtttgatgaagaataattcctagaatctttttgcagtgtttctggctccctttgccatttctcttgtttgctcttcattctgataagggcctgatctttgatgttgacagtctaattgaaccaaaaaatgaacaaataaaatttattgagttcccattatgtgccatgcagtgttctaggtgccagggttacaagaatgaagacaaaagtacaccttgatatgaaacttccatcctacttggaggggagagaaaggtgacagataatgttcaaattaactcgtgaaaaagataattttacatagacctaagtgcaataagtacataaacaaaatgttctgatagagaatgctggggttaggtgttggctagtttatactaagtggttgatgagggtatgtttgaaatggggacttttgagcacagaccagttggcaagaaaggcaaccgttcaaatatgtgggcagagcttctagacaatggggatatgagatgaagaaaccataaagctgaaaggaacttattgtgcatggagaacagcaaactggacagaggggctggagtgcagtgagggagagagctgaggtgggtgaagttagggaagttggcccctgtggctctattctttgtcgtttgctctctgtatctttttcgtacttcagtttgctacatcttcacatttgctgatcttttccacttcagtgtcaaatgtgctattaatccagtccaaagaatttatttttattttaaatgttctatattttagctttagaaattgcatttcattccatttctatacatttcatttctttttttgttatattcatgtcttcactagattcttgaatatatttatgataatatcaattatcaattcatatcaatttcttcagattatattatctctgttatttctgggcctatttctaatgactgatttttcttctgcttacattttctttgcatgacaagtaacattttaggagatgctggatatttggctgttgactgtctgggttttattgtcttttttatggagtgttaaacctgtttgagaaggctgttaagtaacttccgaatcagcttaaattttagaggcttgtttttaaatggtattatagtgaggctgctgaagcccttaatctagtgttagttttgtcttaccattaaggtgtgaactttcaggatctcagttgaatgtgtccggtatttagtgaggcctctgtactctggctggataaaatacaaatgtcttctagctccctgtaaactgcatattgttaagattacagcttattaattggccctttcttttgcacagtcttgttgtgtttcaacctatgtatatgcagttcagtctttagcaactatatagtgagtccccatgcagatttctggagctctctgtctacttagctgtgttatccccagtaccctgccctgcagacaccagacaccttaaccttccagaactctatctgactgcttaactgaggataccccctcccgcatcacagtccagaaagctactgtagacagacagttgtgataatagtgctgaccttctttgtttcccttctttcctggattacagtcctccactgcctttgtctaacatctaaaatcaaccatttcacttatttctctggatttctaaatgtttcattacaggaagggtgttgggtacaagttctttaacatggtcatgaatgatgttgagaaagattcttttcccataacattttattatggaaattttcaaatgtgtacagaggtagagaaaggaacctaatgaacccttatgcccaccatccatctttaacaattagctacatggtgccaaatttgttcacctataccttcttcccagcctcttacccatctagatcataagaaagaatttagaagaatttaaaatggcaaaacttcatgctgtgttaaagaaaaaaaaaaaaacaggagaagatagtatttattgaatctcttctttatttatttggtacttattcttgaactatagatctcaatctgttttattatcacagaaaccttacaacttaatctttttgaatgctgccaaatctattttaaataaagctttgatttgcatttttatttttgttgcccaaagctttacaaatagtctgatagtttcaacagtttgtataccttatgcatcatcttgtgtatacaatgtgattcatatcacatacaatgtgatttggttgcaactgataaaacacagtaacgtcacaattcattggctaccatagatttgaccagtaatatcttgttcaaaaggaagtttgtgttgactgttttttgttatgaggaggagcttaaaaggtaaacaatgaaacaacatggaaaatgttttaaaacaatataatacttaatattaagtgaaagaaactacacaacactttcatctacatttcataaatggttaagaatgtcatatacgtacatataaaattggtataacaggtaatttgaatcttaaaagaaatctagaaaaaaattaagttagtgatttttctaacagatatgtgagaatatattttttcttttaaaaatttatttgacactatttcatcagtaaatacaagttagaaagcattaaaaagaaaataattggaatctattgttaatggacatatccagctgacttaacttactaatgcatgatgataatgaggccagaattgtagctggattgaaaggagaatgacagaaacacagtcaaagccaagcttaataaaaggaagaatcatgcaagacagaatgaaagttggaaaatcagtcgcctgtagcaaaaccaacaaagttaataccactaacaaagcttcacatgtcttagctcaaaatatcatcagtgacaattgtgaagagttgatcatcttgtcccaacactaatgatatttcaggcatgtatctacaagttcaaatgatagtaagttcatagcttattatacttgaaaacaagcatatattacaataaaatatcactgtgggtagtaatttctttactttggaataaatttcacatgtaaaaaccatgtctgtaagttgtgctgagaatgtttattctcccacaatgaagataactttcctgaacaatgtatcatgatagttattgcattatcctctatgtctacttgacccataagtttctcaattatctattattcaagattctatttttaacatcttgccagagaacagcattggcatgtgagtatgcctgtgcttccatgctgtgtatgtgtatgtgtgttataaaatgatggttcgttagttctgttaggtgacaaatgttgagatgagtgaagactgtccacttttccttccaaattttgcttctaggtaagaatgaaagaaatgtcaaaccaaattctggcttacatatagatattttttgaacatttcttggcaagcaaacacacataaaaagtaactctatatttgttctgtaggatcaaggacactttagctcttatgtccttttagatgttttgaccattacctggtatgaaaactccagttgagataaacgtgaaaattccaagaccggattctctcaccttatgtctagcatcaggcaattagaatcttctgtagttcatgactaggcataggaatacttaaaaagacttagccaaacacatcctcttgaaagtgaaagctgcaggcttatctaatttttacacttgatttacgtaagggtggagagtattgctaaaatattttagtgatctacttatcttgagtagttttaacttctattaaaatgagatgtgcgagttcatttctgccattcacagtgctactcaagaggctactgttttcgtcagtgaaaacccatttctgacattaaatattctaattttcaaaaagttgtttgttgatggaaattcagataaccagaagacttttagtactcatgtgagggaaggtgggctttatgatgagaatgctacctttctgttgtcacatttttgaatagaatgctcatagcacctacagtgtagcttattctaagtaaaccatgcataattactggatgtgctatagtatttttagaaaattgcagacataataactgcattaatgtttgtggtattaagaacaaggttatatatgctctagaaatccttatttacagcttctgtgacaaaatgtcactgaaattcttgagtaagtcttatataactatgaattccagagaggtggaagttgttatctttaactaccaggagtgaacatactaccagtaataatagttactgttattattattattattggacatcatggcagtgatggtgacattgttgaaaagtcatagcaataataattgtcctattagtaataatagaggaagaagataaagactagctactgtttcccgaatgtttactctgtgctatacgtttaaggttcacatgtcatttattcaccagaataactcaggtattgttattttatcttaatttttcggatgaagaatttcacacaactggtatgcagcagaaagggaaaacaaattctttgctctctgattctaaatatgtactaatttccattgccgtatattgcttctttttctgtaaaaccttttataagaatttccgtgaaaattttaaaatacatttttgttaggacttctggaacaagaatattttatagagaaagaaaggaagagttctttagtttattaaaaattgtgccagggaaaaagataaaaatgtggaaaactttatttctgtttatgaaggcattcagcttctgtttaaccaggtccattgaaaacagattgaaatgtagtgttttgttgttgttgttgttttttcacatgaaagtttgtgggaaatcttaaaaacagagtctttctatacaaccttgattttccccagaacttctcatgaagactgctgccacagcttttagacagtagaataataaatctaccttttgggtacatagatatcctagtaaagattcagataaatggttttgaaaaatagtttctttttcctttctttttttctaacttttattttaagttcaggggtacaagtgcagggtttgttacataggtaaacttgtgtcatggaggtttgttgtacagattatttcatcacccatctcttaagcctagtacccattagttatttttcctgatcctctccctcctgccaccctccacccttccaagaggccccagtgcctgtcgttccccactatgtgtttgtgcattctcttccacccagcaatcccattactggatgtatacccaaagaaatataaatcattctgttataaaaacacatgaacaggtagtttattgcagcactattcacaatagcaaagacatggaattagcctaaatgcccatcaataatagactggatcaagaaaatgtggtacctatacaccatagacaactatgcagtcatgaaaaagaacaagataatgtcctttacagggacatggatggagctagaggccactatccttagaaaaatagttctctaatgattgcagtttcctggggatgaaccatactcttaagcttacttgggatggcttgtcctcattgattgctatataagtaccttctataagatctctaaagagatttgtattgttatttatattcaaagattaagcttcttgtgttgaaaaatcatttttatgttatgtatgtcaagaattcaggatcttgaatgtaaaaatcaataaaaataagagggcatgcttttattagaaatataattctcaaaatttcaaaattgttgtattacatagttgcatattgttgtagtcatctcaggctgccgttacaaaataccatagactgggtagcttaggcaatacaaatgcatttctcacaggtctggagattgggaagctcaagatcaaggtgccagcatgatcaatttctggtgagggctttcttcctgtcttgggggttaggggtggggagagagagagaaagagagaaagagagaaagagagagagagagagagagagaaaagagtgtgctcctgcttgctatctggtgcctcttgttataaaggcattaatcccatcgtgatgaccctgctcctcatgacgtcatataagcctaattatctatcaaaggcccatctccaaataatctcacattggaagttagggcttcaacatgtgaactttggggcaatgcagacattcagcccataacgaacatcatcagtttagtatcaccaacattaaagtcaagagttatactacttatttatagtttttcactcaaatggagtaacccatgtgcattattaaaattggtgtattttacttatatgaatacatattttgatgtttctgatgatgatgtagtcatgttagggaacttcgtgcttgactagaattccatggtacttttcctaaatatttttgttttatttctgaggttattatttattatttataactattgttatgaaagttatgcagaagatagaagttgaatgctgctgaagtgatcgtagtgtctactcagttacagatttagaaatgcgagccattattatgaagcaagccttataataagatataaggaccaaagagtatagtgaatgtgaaagagttatgagcataagattgctaaagattagtttactatctattagcatttgaagtgtcaaagcattattcagtgtgtgtgtgtgtgtgtgtgtgtgtgtgtgtgtgtgtgtgtgtgtatatatatattttatttttattttttttgagacggattctcactctgttgcccaggctggaatgcaatggcatgatctcggctcactgcaccctgtgcctcccaggttcaagtgattctttttggctaatctgagtctttttgaactgagattgtcctattttaagagtttattttccattataaaagagcagaaaaggccaggcgcggtggctcacgcctgtaatcccagcactttgggaggccgaggcgggcggatcacgaggtcaggagatcgagaccatcctggctaaaacagtgaaaccccgtctctactaaaaatacaaaaaattagccgggcttcgtggcgggcgcctgtagtcccagctacttgggaggctgaggcaggagaatggcgtgaacccgggaggcggagcttgcagtgagccgagatcccgccactgcactccagcctgggcgacagagcgagactccgtctcaaaaaaaaaaaaaaaaaaaaaaaagagcagaaaaaactaactagtctctttatttatgcataaactctaccagttttatttcctagcaaagaaaaggggggacctttgtattattctcacaaaatgtattttgcagaaatatctgaatgttgttgaagtagcaaaatgaactgtagagaggtgaattgttttgatctaattaaattatggtacctaatctaattgtaagataatttttgaactgaagaaattattcagaaaaattccccaaatatccttccattctcacactatacatttcagaatttcacataaatccaaacacttccatttccaggtcaggtcaaaaaatctaaactgtttggtttgtagcagttttcacagacacaggagcccattatccagaaacagctgtcttactaatacttatttctcttaggaggcactccacacaagcagggactcgctggtgttgtgtttgttctgtattaatggttaagggaaggtaaaggggtctttctgatagtttttcctgtggcacagattttgcgctgtggtaatgcttgttgtcattttgggaaaaatagtttttttatatataatacttatgtaatcattttgatacagttctataaaattatatataatatgaaatatatatttgagaaaaataattatctatattatctatatctgtttaggccctgtttatctaaagtattgctaagtcttgctacggtataatattttcctttatggtttgaattaaagcaaatgttaaaggaaaacaaaccctgaagcgaacctcccgatagcttcatagctgctgtttacttactgttaattttattgaagtttatgcttcaacagagcaaaacacagtggtctgaaactagtttaatagaaacccaagtttttttagtgagccctctggacagagctttgtctttaaggtaggggtaatctagtgtattggagggtcctggacttgtatcagaggacttgagtttgaatcttgactgttgagtatcattgttgtgaccttgaggaagttgctaaatatttgaattttcaattattttcctcagtgaaagtgagacaagaatgatctcacaggctcaaatgagaaaattaataagttaattttaaaaactccctttttgttggaaatgcatctataaagtcatacttctagatagaattcacagtgaaaaggaatcatcgaatgtagttgggattttcagatattctaggttttacccatttaaagcaccaaacatttttttctagttgaactattttgattgagcatttttctaaacattattttacatgtatctgctttgtaaaatgcacgggtattattgccttacatttgtatattcattcattaagtctttaacaaatatttgttgagtgctattctgctccaggcactgtgctaagccttgtacataaactgatggaaaaatggacatacagagtgtatgaactagtaggaagacacgcactcaagaggtaaaacaaacacacataaatgatacacacattgtgataactgctaagaagaaaaacaagagggcactatgagaagaaacagaggggaaaaactactgtaggttgagtggtcaggaaagctctctctgagaaagtgaaattgaagctgagatctgaaggatgagagccagccgccttacagaatgtgggggagaggattccaggtactggaaacgaggaaaagaggcctcgtaagaaaaaaaaggccctttttgttttcaagaaactgacagaaattcagtgtgactagaacacaggaaacatggagaagactggtctgacaaaagatacagaatcctgtatagtttataaaacatatttatagcttaaaaccagttgagatgcagaagactttttttgtttccatgtatagacactgagggttaagggggctgacttgcccaaggtcaccctgctgggaggggaggatgaaagctccagcctcctttgacccctagtcctaggctctttgtgctgtgaaaatttcctagctgaacagtttcatacttttctgatgtctgaggccattattatgtaatcagtcactgaaatgaaaccctaatgggctgttgaggtgtgaaaggcccattgccctgtaagatcttctgaattcttatgtctgaatcatagtttttctgtctgctgttgggttgctctcatttgatttcagcaattcccttatctgtttttgaggagctgcagacatagaaaccaacatgtttagtttattaggcacgtgtaaagtaatagccaatggcttctgtgtgagagcccattggattgttgtaatatttgttgtctcagtaggacaggcagtcatgtttgactattcctaattgtaaaattgattttactttgaaaatatttgccaaaaaataaatcatgtttaaatgagggagatatttacataaagttgttgatgtctgaggagccactcttatcccattctcttaagagtttcaaatcaattgtgaataatcacaggagttagacaaagatgacagtaccaggtttattgttaataaagccagactggacagctgtccatgaacctaaggcctaaatcatgcttccccttctttacccaagctgaacttaacaaatgcaatcagacctctgtctccagaaccagaagacagagaaagaacgctatgtacatctggaaggcaggctgcatctgaagaaggtagcccaggaagggttaatgaaacctgccctgtgaccttttcttctgttactcatcagaaaggaaagaagtaatacctgatactcacaagaaagtttttattttataaaatggaataaaaatatgagaaaagagagaaaatggtttcttctacattaaggtaaaagcaaaccattatgatttaattagacaacatttacaagtaaaggtattatgtcttgtgaacatacatacttagtatcaaataggataatttcagtgttacggcttagtaggctaggagtcagagacgacttttacttactttattatttaggagaagccgtagaagaacagtgagctccagtttcgtcatctgtcataacgagtgctataaatacatgctctgtctgagagaaacagatgctactgtgactatgaaagggtggtaggaaaagagaatgacatctgagtacctactgtatgccaaccaatgtgttagatgcttttcaggatatcttgtgttatcaccataatcaaacaagtgttattttcatttttcaaagaaattgagtgtcagattctacaacaaccattttttgagcatttatgataagccagtctctgtgaatacagtggtgaacagggataccacaagaaagtggcatttcaacacaggtatggagagtgaggggagctgggcacatagagggagggttgcctggtcgtgttcctctctaaccagctttcctctacaccttcctcagatggtcttcctggtgtctatttagtcagaaaccgggatggcattctcgatgctcctctttctcttatcccatacacccataggtacaaaggtcctgaaatagcatagagattgggatgtttatggaatagagagaacatcatttcttgtatgctttgagagttagaattgtgtcttaagtgcatagggaagacacagaagcgctttagactggaatattgtgaacctactcttggaagaaggcagttctccatgggtctctcacatttctgtatgtgttatgagtaatacctgatactccagagttcatccagactagattgggtcactggcctacttatcacaatgggtggatctgaagctgactgttagattgtcaaaaacatctccatgtccattgtcgttgtcacagactcccttttcaaggatgtttgtgaacaactttagaagaaagtcatagtacctccctccatagcaaaagcaggcatgcttactgtccagaatgataataaagataatgtctttctttggaaccaagggcaggcttgtttactgcccattataaaaaatatttgatgtccctacattcagggtcgtttgctgcaatacaatcaattgcatgtacaggtgtcatgtcaccttgagggaactggggatcagagaactgtcacaagaaaatgctgatacctgggcttcctttattggaagtaataagtctcattattgtgagtaataagtctctgaccgagaagtcttgtgtatcctgccagcatctgtgaaattgtggcaggctaacttattagcttgtgaataagataaaactcaaaccgttcgtagttgttggcagttttagggatgaggatggggtcctgatagagacataagtttctggaagagaaagaatgaagacctcctgggccaattgatgagatttgagaggaatccatggacatcattagtgaacatgttgatcaacttgtttggtcagttagggaataaatgaccctccattttattatctgttgatgaggaagaattggagagatgattgtaatccaagtacctggaaataggtttaaagtcagctgtttgaaaggtgccctgcatgttactagccctgttccaggcatggagatgtttttgacaatctaacagtcagcttcagatccacccattgtgacaagtaggccagtgacccaatctagtctggaggaaggttctgaccctctttgtaacaacagttataatttcatgctctggcatacaaggaaggaaatttgtaaccactataggtagaaaccaagcaaatagctataaccttgggtggttcccttagaagatgaaggtgtggggtatgtaatacttgttcagaaagtattaaccctcaaggttcttttagttgcaccattttacagcttatcccctcctgtagactatcacaccaaccttagattcactaggagacttagaatttcttagagtggggccaggctgctataagggagctttggttaccttgggggagatttccttgaaagagaaaagctcccataggaagccatggatgaggtattgactagtctttgggccgttattgtcctagaatgggtttgtaatgctgatgaagattacctattggaaaatgagaaactgacctaatgaaatctagacaaattcttaccgtcacccctgttgcctcgcaaaactctcataattcttttggtgaagcagtgatagaacatgagatgttataataatggggcagtaaatgccctcaattgaaacagactccttaaaaaaaatagctgctgcgcaggtcaagcccaaatcagacaaatggaacaaattctatagccacagccagaaagtggttatggctgctaagtctaggggttcccagaactggaatagatggtgtctgtaatgatcttgctgccaaatatagagcattgcatggaatgaaaatagtagaagtttatttgattgagccctgagccagctgccaatcaaaactgaaagtaaataaatcttgtttactaacatagaactgttctctccctctctgcccctgattccccctcctcggactctacttcagctttagcttctttaaggaggaaagaatggagctgggttctctctgtcctgaaagaggtccacagttcttacatgcctctctccaagtatgctgggaaactctggcgagggcagggacttaaacttttatggctctgtgagatacacattaaggaaatattaactggcccaatttatcatcctgcccagtccgatgaaggaggggatagctgaattcagccaaggtggtttgaacatggtttgcagtgggggaaggaagctagcagaccttacgggtggggcctttgttccaattagtgtgtctttgttgtagcttcccctgctgaattgtagatgactgtactatggatgtttatgaagtccagaggggagtctcctgaccaggaagagttgcaagtacagaagtgcttcttcctttggggccccacaaccctaatgacctctttggagcttcagttctctatgactgatggtattgctgattggagcctcttgcaaaaggaagcagcattcacccttggagttctggactcacagctctgacgttgaaacaggtatattcctttaaagaagggtctcttctagttaaactgaatgcctggcccgtggagaccttgtaactcttttttttttttttttgacggaatcttgctctgtcacccagtctggagtgcagtggcgcgatctaggctcactgcaggctccgcctcccgggttcacgccattctcctgcctcagcctccccagcagctgggactacaggcgcacgctgccacgcctggctattttttttgtatttttagtagagacagggtttcactgtgttagccaggatggtctggatcttctgacctcatgatccgcccgccttggcctcccaaagtgctgggattacaggcgtgacccaccgcgcccggcgagaccttgtaactcttgagcctgatgttcccgctttcgggtccagtcatactcattgaataacaagggaagggcccaacaggcctgggttgtcaaatggaaatggcaaatttaggaatgcagccagcctgaggcaagcagcatcttaacctcacttgagaaagtggcagctctaatctcccactctgctgcctgattcaccaaggttcccctaagtacctgggcctgcctcacttatggatcagctaacctgaaacctaatggtatccagtgggttatgttcagcctcaccatcacctgctcaactagaaatggacattgtttctgtgctcagtgttcacaatcaaggaagtgctcttagccctggccaatactccccttaatgaatcttgctgtgtttttactgtctctgggagtgttgcaagtgttgccaatgacctaggtgtttgggctgccacttgaaaaaatgccaactggaagattataaactcccctctttggggccacaagctgtggaaatggttgggctgtcacagttatttatatagatggtaagggcctattctctgatgaaaagcaatgggaatcaagctgctggttccacttgcaccacccagattgccaccactgctgcctcgcttcatcagggtgttggacatgcgacacacccacagcacagactgggcacaaggtgaaggacttctgatatcgaggtgactggcatcacagacttggggctcctgccaatagttgacctgtttgtctcaatgaaggaagacacattgtatgtacagggagttacttctgctaattcttaacagatttactgcatttgactcctcttgaaactggcggtggtgcctcactgctgttgacacattttcaggttatgagtttgccaggtgcattccagttagtcaaagccagtggtgcaccttttattgtagaagccagggctgatagtcaaagtatttgatagtctttccatgctccctacagtacataggcactttgtattgttgaatgttggaatggcttcctaaaatcaactcaaaaagatttaaaactctacctgcctaatctcctcctggtttggacacgttaataagacagttgggtcattaactgcaactggcccagttagggatcatcctctttttgtcctttcttggttatgatcaggatgaaaataggttatctagaactattttgaaaattcaggatatcatcctgaccatttctagacatgatgaatctttctttctcctaatacctatctcaggctagactgtttggtacatctgctgggtggcagcctggtcaaaagatggaacaggaattcaaatttattctggttcagcctcttgagttttcttattggattgcattatcttaggatattgatcgcttggttgggtaaactgctcaccaagcgcccccaaaccttgtatccttgagtcttcttcctgtattcacgaaacggaggctagctaccatgttagcttgcacatagggtaaaatctcagacctttcataattcttgacatctacttatgtattgaaatagttttagccacttcctgccgaatggattgaagagttaatggtacaaattgggagtttatcagttgtaccaggggattttaatttatggaggtgactgagtgacaacatggagaggccaatgccatcgaaagattatttttattacttacatttcccaagaggagggggggcacttacattaaatgtaagtaattttattacttacatttcccacgatggggcgcatggggaagcactgggtttggtcaggatgcagaaggaggaagggaaagcctgggccatatcctttagtggggtttctgtgggaaagggaagggcagaggaaacagtttaggactggctagtttgagtaatttcagtgggctgtgggttgtaggggtgatacctggttgtctggtacctggccctgggagatttagggaaggggagtattgacttactgatgagaagttgataaaggggatagttgagggtgtggtctctggattggttagctgcatatgaagggcatgatcccagaccagccctttgcactaagaattggctagctctgggagtatagtctcttcccattcagtgaggccacaaattccagagcatcggaatacagaaaataatgaaatagagttgatataactggccttgtgataaatggatgccaaatagacaaacacaatataagaaaacagttttagaggagagaaactgcttaggggattactgtagtaatccaaataggagatgacattggttttaactagagaagtggctgaagagatgaagagaagtgaacagatttgagacatattttgaagcctgcatcaattcaattgatgatgaatttagatatagagtttgtggaaagggaggactcagtgactcctgtttccggctttacctaaatgaaacattttggacggtaaatatagcattggtagagaaataataaagagttcagcactggacatgttcaagttccatatggctgtgagattatttatgtagaggtataagttaggcaggagagtgagagagtttagaactaagaggatgaatctcaggtggtgataaaaatttgggaacctacactatatagacagtagttaaggcatggcactagatgaattatccagtagagtttgaatgactgggggcagatcatgcatttgcccacagccccacctccatttgacctgcttctctcttttaaataaccatcttggcttctgaatgtatgtgagtttgtcagccttaataggcaaagaaggaagacagttaagcaaatggcaaatgaaatgattgcattgatttctacgtggtatttgttatacagtggcatggacatgatttgttaagtgtttaactaaaagtagtcccttaggttttggattttatattctatgtagataacacatcagtagcctggctaaggaagctgtggtggctgtttagttttgatttcatgtggtctttttcaaagtagcaatttaggggcttattgaacatacagaaattgtacattgtttttcttcgttctctaatgcttgccagtagttatcagaattacatgactatgcttttgtttttttctttgttctcccatgttggctctgttttatctcttaatgtcaaatgtcatttcttggaatcattcaggaagttcttttagttagtttgccagtaatgaaaagtggcaagaaatgctgtaaaagaacctggaggtatttcacagctttggagatgaagggtgcttgatagattactagacaagtagtcttactaaactgctcaccaagtgcccccataacctggatccttgggtcttcttcctatatccgtgcaactgaggctactgtgttagcttacaagcagggtaaaatctcagactttttgtgattcttgctgcctacttatgtgtactcatgctgagaagagtagaagacacgaagaaaggaagtatgtaagcagcttccttgtgctaagagttatggagagggagtagacaaagtaaatgtaaaggatgcaccccatgaaactgcagtaattaatttattttcttgattttaaaaatacgtatatatgtggcaagacttctaccaaacttaacaggtgtcattactgactaaatcttgattcgcatattactttgaaggatattcatgcatccaacaaataccagtgtagtactgacaatgggaaagtcactgctatttaatatatcagctgtcaatagttcataatagcttcacttttatcagaatgtgtggctgactctcatagattgatggactcaaaagcactgtatagaggttttggaggtcagggtgccattttcaacattttaaaacaagggaaattaagaatttacccatagtaaggctgtactggtttttaagaatatccagttcctttgattttggctatcttattaactattaataattcgtacctacaggatgtcataataattagcagcattgaatgattgctatgtgcttggttttaaacatggggtaatgaaaaccgttattgttaaataaaatatatatggaagatggagtctttcagaatgtgtggttcatattcaaaaataaatactggcactctgagatggaaacctctatgtttgttcccaggtcacataattaattggtggcagtatgctggaaccagaagggcatcctagacatctctgtttataaaatgttattaaaaatcatctatgcaattttcccacttggttataatggtacaaagtaaagggataccatttctctgtttctaggaacactttggtgtcctggtgtaagctgagataaagattatatttctcatcagaaaaattatagttatttgtactctttaaaaattgaaactgcaaatgacatttgttttcacgtttatatttataaagtcttcaccacactttgtttgttgttcctacctcgccccgctttaagattagggaagaaaattacaatccacccaggcttctttaatgcatgaagaacttattctcccagagttttaggcaaggttcactgggcctctattgtaacaggaacagacatatattttgcttcagtgagtgtttcttgagctcataagcattttgaaaatattgaatgacttacagtcccttaagaaagtagccatttaaaaaagcatattcagttgaactttaaaaatataatttataccacaatactcaacaggctccaactgcttgaaagtatttggctgcaaagacattatttttagtgatcctaaccatactaggaacaaagaattgcgtctgtcaaaatgggatctctggcgctctaaaataatccacatgctttgtgaactttgatgaagtcaggcagctctctgtcatataaatatcatagtggcatcaaaacctgggctgaccatgtgctgcaggtggaatgcacacactcacccagtttcgcagcagaagcagctgacactagtagggagacagcgccgtcttcagttcctaaagccattggggcgtctgtgtgctgtccaaacctgaaataggtaaatgattcttcattcaatcctcccttgaaaatgaaaatgacttcagtaaatacttgtgctacaaattactcagatgcagtatttactcttttcctgtacgtcactgtatcatttagagccaaactgtaaagattatgagaaaaaaagaaataaactcagtgttagataaacaaattagagacaggaaatgctacactgaaatactgtctgaagaacttatgaaatgagtttaatctttcttttaagatctgttaagacatttttgaagggatgagagatgagttggaagatactgttttctttaaagaaaataattatttttcataagctattagaataaaagggaatgttaatatcagataatagcattattatctgattttattatctaatcattaagattactcacattatccttatctcttgaatttatctattcttgtttcattttcatttcattagaaattaatatatccctctctttcgtaaacataaaagactttattttataaaatatattacataaaatgttaacatctgtgtgagcatttataattgtgttcatcccttcagtctacctcagatgaaaatatatacaaaataaattaaataaaaatgtctgtaattatctttccattttgttgacatacatgtgtacctaaaactagaagagagccctgtactttttagagtgaagtaataatgaaagtgaaatcttacataccttgtcttatccaaaaaaacgagaacaaacaaaaatgtaaaatgttggtagggaactttaaatgaataccatccttattgatattaacttgccataaagtgtttctcctacagaaatgttttgctttaaagaaatttaaagtaaaatttgctttttgaaaatttaatgcagggatttaattctgagtggtttacaacaatgggttatgaatgtatcaccatgacagcaggttgctagagtctaggagtcaggagatgtggcttcattaaaagcacctccagataatgccctacgttagatttcactgaaagacaggagctgctgatgagggtgacctctgacatcaagtcatcttactataaacagaagggcgtggaaagccattggtcggaaccaggagtgtttactatccaggtctcctcctatttaacctacataactgcactgctcctggagcccgaaggttcacgccagagcaaggtgcttttaaggctacctgggtgttggttagagactataaacctctcagagcattctgtaaacctcgacacagtttcattctactgacaatggagactggaatttattggctgctgaacttcacacacacgcactacagaactgacttccaaatacagctggaataaatttcaggttttggggttttgaatgaggtaagttgcatttagcttttggttgaagcgttatcattattattatgttccatgggcagtcacacatttaagtgtaggcagttctatctcagagaaaaatatttttcttgttaaaataggttgtttagatttttcacagttttttagtacaaggatgatagaagaaacttgtacataggaactgagcaagccaatcaattagagaaaatccctgatctgtttatggctaaagtgtgaaacacacttcgtctttgtttactttataaaattcagctattcaaattacattatgttaatgttgggagaaatgcaggtaacgtgataagtgataatagattacatttcactacaaaacttttaacagtcaactttgttttcccctaatagtacatgctactgcaggattttaaaaaactaataccaactatttaaatgaaaggcttttttctgcataatttgttttgaaatttgtgctgaatttgaacttttcattagatttgagatgtagtcatcttgatagatatgtcatatttttaagagaatgagtgtttccctccatttatggaactattttttcctgaaaccataatagtaagatcaagtttaattttaactttttaaagaatcctgtagtgtttagtgaggtttaccttcctttagatgtgtactgagcacatactttgatttaaaaccttgatgtggaaggctttggtatgctttggtttaaattatttaaatatatctttgtgtctttctcaagaagaagaagtgtaaattgctgttttgctgcatcagagggttgagatgtgggagaattgtcaaaataataacagcatttgttttgttaaagacgactgagggaagctttgctgtgttgggactattttattcttgtgagaacaacacaattcaggcggagaaaatagatctattctaagtatttgcgagtactgatttactgttagaggtggatagttagggaagctgctggaagaaagggaggatagattatcccagccaataagttttcccgtttggtttacggggtctactgagcatcagtcagcactatccagtttcccacttcttttctgagttgtaggtactattttctgaaatactctgcaattgaataatgatgttacactaccttattttgcgttttgtttgttttctaaccgaaagttcactaaaccactaaaatgttacttatcaaaaatgaccaagaaaagcaaataccagtaaccatataatgggttctaactttttgcagggggaagaagagggacagagaggccaattttttatctctactttggtttttctcttttcttctccaagttccttataaattactctcgttagcagtttcaaaattgtatgatctaactttttgatcctgtagcaaccctgtagagggagatggttgtgtgagtccaggggtggcacacattttgctttagggaaatggtgcaagccaaatttaacaggtaatgcagctgttgaagaaatagaaaagccagatgacttcctacgtgtattgaggaaaccaggataaattgtccttagaaccagctcccagaaagccattttaattgaggtcaaagaggaagaaattgtgagtatatctcaattatttattaggtttacctagaatgagaccttcactacagatatacctaatctataattcctatactctttaacccttgagcattcctgctgtatgaatccctttaacatttagactgcagagtgtatttaattgtttatgacagtccagaatttagggacataaaaattgtgaaattaactgactaaattttactttcatctaaggaataagcttctgcatataaaaattattgtaataaatttcttagattttagttgaagttacacagttaatgatgctgtttcttctggtcacatgcttgtatgtgtgaaatgtttcacatagaactattttcagctcttagtttacaggtaatttaaattgttttttctttaatttgacaatttgagtatttctactcaaacaaatcatataatttgcattcttggtgtgtaaattatgctacagtcagtgtcttattaactactaatgaagaattttattttaggattaaaacctgagaaaaggtgatttgtaatctttttttataacaatggttatagaagagagatttaatataaaatataaaactactttaaaatagattttaaaactcaaaaatgttgctacgatgttgtggaaaatatcagattcaatccagcatgctataaatacatttttttttattgtataaagcttaattttaaaaataaagaacttgatgccaacattcttgaattttctctttataaatgttgctattttaatggaagatcaagaagataaaatctgtaaaattcaaaaatatgcctgttttacataattattttatcaatgtattgttctagaaatatttatattttatataaattaatctacagatatatagagatatatgttgttaaatttaaaattgctttaaaattttgtgtgcattctatcaatttgtttttaaagttaaagaatttctaatttttttaaaagttatggtgatactattgggcacattaggagagtttttatatatacttttaaaatgttttttaaagtttaggcatatatatgtacttatacagacatacgcacacagataaactttaacaaaacttttgcatatgaattgaacaattttatattggtaagtgagacaacctttatagtcaattattgttcaaattgaaaaggttagttgtcatttttacttaataggtaaatgatggtgtactagagtatattacataatgattgatattttatgtttggaaacatatgtaactatgcctgtggccaaattcttttgaagtttaatgaccaagatgagctgcatttgttaagtggtagagcactggcttgtctggggtccttttgtgcttggcagatcttgataaatgggtgagtttttcttaaccttgtggccaaaacattccaatatctttaaaaaaatttataaagatatagaatatgttgcgaaaaacatgagcaaattttctctatttaggggactaggttaaatttttaagatgatttttaaagcaatatttgtgagaattcataaatattcattttaatgcagggaaatctaaagtttctgatatgtaatactgctggaattcttctttcctggttcactgcgaagctgcacagtagtcagcaattttaatttcgctgcagaagattctaccaaattaacattatgatcattaagtgataattctttagaatattcattttgctctttttgctatgctaatacattgtaaagcatttatccatctttcttgtgacacccaggacggcaaagaaacatataaacaaaaggtataaagaagaccaagatatatgaggaagtatctctttaggtgtttgtttatcatgcacatttgtgaggaaaaaaattcttttagcgcttatgtatagtatttatgcatagtatcaaggctggacaatctagtcttttttccaatcttagaatgggagctttcatatgagctgtaacatgatatgttaaagcatattatggttccttttgtggcattttttaaaaagtaatgtgttacctttgcctgccccactgggtaataatgaaaaaatataccattagaaggagtttgaaattttcctaaataattcataaagccgatatggcatttatggcaggtgaaaaaggactgtggcatagataaggagaaaacgtgctttggcattttaaaggttaatgtgatttgacatttggtttaatacttacatactgtacataaataattgtgctttgtttttctgttattgttgctaagttgctttttgaatatattcaaccttgctatgaactctcaaatcacttgaaagctattgtgacagttgtagttgatgaagagttatagactatagaagtttttccacttaaaacataattcctccctagtgcatgattttgtctaacctccctcccccaaaccagggtcctggaaggccctcgcattaaaggccagaaatcaagtgagatcttgcctgtgacagcccaatactaaaggagtaaaataattttgccttcaggtttttggtggtggattttatttattaattatctaactccactattcttaataggtatgttattttgtctactgttgcacttagagtatttgatagtatctttgatgtgtagcagttaaagaagggttatggcaggttttctttgaatggaagtagatgcagacttccaggtatgcagtagagtcctctcactttcatagtcaataaatggtgatttgggcttttagaaacacggttttggttttgccagggagaagcttggttaaggaatgataaagaggttgaaaagtgatatagtaagaaaatggaggaataaatagacttccctaactacatgaaaagaatttcagaaatgtatacattttacagctgcaagatctttgtttctaaggaaaacaaagacactttattaaaagttttgtattatttaataattggatttttagaaactgtatttctaagactttaattttttgaccaccatttggcaaaatgccattttattttccttgaaaagcctctcaaatttgtataaacatattaaaacagagatatattttttaaaactcttggtgacaacttttgtgttgcaaagatttaatgtttcgagtttttaaaagttaagaagtgaagtataacgtaagtgaaagatagaaaatgagtcgtattgagaagtgttgacacatttttagccagttgagaaagtgcatctccaagtttatatccctatgggcctagcagtacaccaatcctatgaagatcttcacaacataaatcatatttacttggatccaaaaataatcataggttatcacagtaaagtgtacatcttatgggaagtccttgagtggtattaacttttaaatattatatttaggatatgagtatatattttctttcacacctgtacgtaagtaaactttggaataatttttagtaaaagcttatattgtaaaaattgtttatgttttgcctatttcatttattacattaggactcaatttttaagttaatacctacttttacatttttttatgttccttcatacattcctgaggtatgtaataggaaaggacatatgtaataggaaaggatatattttaattcatatgtattaaatgctgctaactagaaaccaggataatctagggcagcccattaaaccctgtatcagtcggttatcaatttttaaaaaaattaatgtgataagggccagtcaattgagatgcattaaataagaacatgacagctattcctggaataaagctggaataattatttaaaatacttttaaattacattatttaatttaaaattcaaatatggataacttgtaagtattagaaaatatgaaaaatgtcacatagccatggacatttcaagaagtcttttattttcgctaaattagaagccaaaggtaactaaagttagataccattctgcttaatcaggcaataatttacaacaattttattcaacccagtcataaaattcatttcataaagtatgcattcatataagactctaggcattgatatactacagttttttgaaataaatatcacttattcagtcagattcacatcttagagtattcacattagaaatgccaacaaagccgacaagtctaaattgttgttcacagtaattctgtatcgttttctatgtttacaaagagtttggcatgcaataatctgtaatataatctccatttttggaaaaaaatatttttctatctgcttatccatgttttccttaagcaatagtgcatttaatctcatttacattatattttatattttctacacagttcagtgaagaagtagtcttaccatccataagaatatatattaataaattatttattacataaactttcatttgtgaaattttaatctcgaatttatagttgctgaatccaccttttgttttcagtgtatagtatgtcctaagactcaattattaatgctcatttcaaaacaaaatgaagttccaaaagagatcttttgaaaatgtcttcttacattattctattctttacattttttattcccattaatgtttcacgatttgtaatcagaactattcactagaagataattgggataattgagcccagctattttggaagatgctagatataataaaagaaccttgctcttatttgcaaactttttttatgagttaattctcctcaaaagattgtgagcttctcaaagcacctagtggtttggaaacattaaaaaaattaagattgatttttatatatgaatgtaatagtcataattccaaagagtccagaaattatggtattttctacatatattttccagatattgaaagtattaattattctcaataccaaatcacacattctttattttaaagtatactttatgttacttacgtaaatacaacctaaaaagcttgaggggtgaatagtattgtgaaataagagatgcatagtgttgtgtaataagagaaatgacatttaaaaataattgtgtgactaatgtaataatattgaccagtcctctctactcctttattctgctttattttcatctcagcacttaaccactgctggcattgtattgttcatttaccagtttacttatttcttgtatgtctcctccactagaggaggaggtgtttgttgggcagacaggtgtttgttggtctaggtgagtgctgttcagtagaaaaatgatacaaatcctatgcgtattttaaattttctagtagtcacacttgaaaagtaagaatcaacaggagaaattattttatttaacacagtatacccaaaatatcattttactctcaatatcaaaaatactaagatattctacttcctctttttcatagtaagactttgaaatctggtgtgctttttacactaacagcacatctccgttcaattcagccacatttcaagctctaaatagccacgtgtggttagtggctcctgtgtcaaactgcttacgtctagttcatctctgtggctggaacactgcctcggatatctcaggcattcagtaaagccttgctgaatgagcaaatcttgatattttggacagaaaaagaaacaacgcagaagttgtttttcacattagtttttttttttttttttttttggctgtttttggggtggtggcggaggtggatgctagagatatctggataattttttcaaaactactatttattttgcttacatggagtttaaatgttactagtgcttggtaaaaactggttacttcagtagtctagtttaaaggattcctttaatgactgatgtattataaaagtaactagtaggttcttctttgaaaattgttccacagaactttaatttccactcttagttttaaatgggagatattagtcaagggtacaaagttttacttatgcaaggtgggtcgttcccagaaatccactgtacagaacagtgcctatagcaacaatataggcactaatattgtatactttaaaattcactaagagagtagatttatattatgttcacacacacacacacacacaatttgggaggaaatttttggaagtgatggatatgttcacagcatagattacagtggtagtttcacaggtgcattcttatctcaaactcatcaagttgtatacattaaataggtgtacctttttgtatatcaatcatacctcaataaagtgagattttaaaaagaaaggaaagtaaactaatgaacacaactggcaaatgtagcataggagagaatgaaaggcttgatgtgtggccttaagttgtaattccagccttgctagtgttagctgtgggatattaggcaagttgttgaacctctttgcacattagttttctagttcataagaggaggattttattaatcatacctgtctacatgcattaaatgagattatatatatacatagtctagcagagtgagtggaaaagaatatttgcttaataaatggcagtaattattattgttatgtaattgaagtcaaattttaaatattaagacgttttgatcttcattttgaacatttttaattcatatgtttatgaattttaggttcttcataggttctggttaaaatgccttcatagatctggagttgaaatttgctacataacttgctttcaggttgtttctgcttctcagtgttactatatgggaactgggaagaaaattgtatatatctaagcactgtgcctggcatgtggaaaacactcaaaaattttagctgttattcacccattccttcatctcaaaaatttagaattttgatatttatgttttcattgaatactaatagttgcttttcttaaaatagttaaatatttaactctttttcatggtgcttggaattttcctgtatttttataatttctctgaagagatattgcataggttaaagttcactttatttgcagcaagttgcattcatttacgctattcatcctttctgtcttggtgtgtgtgtgtgtgtgtgtgtgtgtgtgtgtgtgtatttctatggaaaaattttcaacatttatttttttatttattggcccacccccttgtgtgtcttgtgaaaactaggatttgaatttaactttttggcatgaaattctgactcatttcttgacctgcttgttttccacttcattcaaaacacattcagaagtaagcctggggtgatggtgcatacctgtagacccagttacgtggaaggctgagacaggaggatggcttacactagggagtttgagactgtagtgcaccatgattgcacctatgaatagccactgcactcctgcctaaatgatagtcattgaaggttctatatgttttataagaatattctttcttatatagcatatatgggtcagcacttgccatttaatgaagtctcaaatggaagagtgtgttcaaattaggtaaaactatccatttgcctctacttctttgatcaaaaccctagaaatatttttcttgatgattcctttaactcctctaccttaattcagaaataagatagggttttgaagtaatttgaagatgtcataaaataaccttttttgtcttcggtatatctaatctttcttttcaaatacagataaaaagatctgataatattctagttaacacttcaatcaattacaatattctgtcaattctatctcagaagtgtttgtcaaaaacaccctctcagttcaccccatggctcctgtcttgagtcttcaccacctctcactgacagattgaatggttcaaagtagcatttggggaacacctactcagggctggggatataatgatgtaccagatcaatgccatctctgccttcatggaacttctactatccgttttgttgcattaggcttttcccaactttcagccctcacctcatcccccttccacattaccttctatagcatctctacccagtttgtttttttacaacctttcaatggtcttcactgaccaaaggttaaattccaaattctttaccgtatataagggcttcataatatgcttcctctggcaaacttcttgacactgaaatacctactatttcccacatacagtctctgaagactcaattcagaacttactttcttcattgccttctccatcttcctttggctaagttaatttgttctatgaacactcacaatactttagatagacttcccctagagtagtcatggtatcctaaatattattcttctctcatttgaccaagaacaaatgaatgaaacctctaaaaataaccactctgctaaatggttattttaatatctaaaggaatttctaattcattctttcatcacatgcctgttaagcattggttataagtgtgggactctgtgggtactaaatagcaaaaccctgtagatgtgttctctgcctttactgttactactttgagaataaattaaaacaagcagaactttctagtaagttctttggggtaattatgctttggaaagccgtgggtgttaaagactaaaaaaagttaaatgataggaagcttgtctgtctctgactttcagttgcgagcatatgtctagtttgtggtggtctggagcttcctcagccagtcccaaggtccagtcatcatggtcttgaggaaagtgacattcacctggggataatttctgtgtattcattgggcagttcatatagttttttggtgataaatcataggcaagaaccagtaggaaagaaggttctttttttttcagatatgctttccttttaattcttgttttcctcatacaaatttaatggacataagacttgtgggatttggagttaattttatgtttacatttttaaaaaagtatttctccctccccaccctttccccttttctcctcctacgccttattcctcttcctcctcctccttgttcctcttcctcctcctcctcctcttcttcttcttccctcccctcccctcccgttacttccttccttcctttttcctcctcctttcttcctcttctccctctcctcctcctcctcttctttctctttttccttttcttcctcttcttttcctcatcccctcctccttctctacctcctcctcctttttcttctcttctctctgtgaaaccgttttttttttcttttttttttttttttttgcccctccatctcatggtactgattaccatgttaaaggcatagaagacattacataataaaacaggatgacagtctctttgctggtcagttttattttcatattgtgtaagtgtatacacaagatgcaaaagagactacagagttaatcaagaaaataattgatttcccagattataaaaatgtctagactttgtattaaaagcaaaagacgggatttttgaaaatgtagggtttttgtgagtttgtttgttaaggatttttctgaaacagaggaggaaaaaattactttgtgagttggaaagacaatgctgtatcttcaaagcttaagacttacttttaagtggtactgttcatattgcaattcacattgcacacatgttcctcatggtatatattcagtagaatttttttttgcttccaaaccacatattggaagtaaaactccttggattccataggttgcatagttaagttattgaaatatgaacacccagaaatagtgggcttataaatgtatattgtacatttttcatattaaccaagagatttttgaagtttagtctactctgcatatacttttaagaccataataatcagatatagatataagtgcttgaagtgcaattgtgaaaatctatggatttcaccatctttagaggatgtaccacatacttttgtccagaaatagatagcatggcctagaagtatgagttattctcatcctttacctctgtgggttaaaaaccacaaagttgaaatatagtctgcaagatatactgaaaatatatgtgtgaagcagtgctaaacttaattgatatgttgagacatatataaaacatacagccactagactgagaaaatgaaaattagtgtatgaacattttagttaatgctgtgctttgcatcaaaacctggtgtttattattatgtgcttttagtaggagacagaagcagtatcagttggaaaaacatttcttagtggattgagaacagtatgctttctagttcctaagtgggaaacctggttctttggtttctttgttcctggttaggcttatccccagttatttcagctgaacattttacatactaatggagtccagggaagtgtatttggcagctgttcatgggataataatgaaatacactatttagtactgtatgctgctgctgtaaagtgtggggggtattttatagagaacttggtgaacagagctatatccgctttaatagtgtgtgaacctgagtgaggaagttgtattttcatatgagaataactagtactcttttcttacaggtaaagaaaacgtgcacagaatcagatgtttcacaatctcagaattccaggtacagtaaaataaagaccaagactcccctaaggagaattgcagtttcggagcactagtaagatgttatacctgagacacaattgtgatttgaaacattgagcttcttgaagaacttatcttgatggaaactgtatatcatgctggctagcgtaccactactcataatattagtcaaagggactgtgattgaagtcagttttaaggaacaatagtggaagtatctctagaaatacgtgaaaaaaaatcccttggcattaagttaatgttcatttttttgttctaatttctttttataatgttttgaatctaaaattgtgccagatactgaataactgtcaaatctgtgtttacaaaaaaaaaaaaaaaactatgatatttctttttctttgcagtgaaacattgctagattttgagaatgttgtcaaattaggaaactcatagttaatttggcattctcgtttgggttgtttggagcctgtggaaatgatgaattaaagtggtgaggccacttcatataggaatttagcaccatttagttgtttgtggttgaaccagatgcccaacagatttttttagacctacacattttcattgctttcatttcaaagtccaggctgcaattttgaatggataagtaaaagtatcgtaaatactatgacaataataaacacatgttcttactatgctatgagtgatatgacttaacctatgtgtattcagttgagagatgggtctcgttttattttgtcctacttgctcccgcatatagtgcatgtagtgattttcttggtacatgaaaacattgcatatctaggtaaaatgttaatggccaaggccaaggtgggtggatcacctgaggtcaggagttcaagaccagcctggccaatatggtgaaaccccatctctacaaaaatacaaaaattagccaggcatgatggatggtgggtgcctgtaatcccagttactcgggagggtgaggtggaagaatcacttgaacctgggaggcggaggttgcagtgagccgagattgtgccattgcactccagcctgggcgacagagcgagactccatctcaaaaaaaaaaaaaaaaaaaaaaaaaaaatgtaatggccattatgtttttctttttgtagagtaagtgtatcttatgtatagtccacttatagtttgagtaactcttgttttgttgtatgcagtatgatattcctataattgattcctgtgcatattatattaaaatgattgcatatggattttccctttggatcatgaagagcatatgatatgatatcagatttttgatctttgatctttgttctgtgtaattagctatcaaaatggaactaactaattttagattattggagcattaggaaatggatgaaccactaaatgttttcctacaaagcagagtagttctaggatgtgcgaacagaagggaagactggggctctattccttggaggcacctccttcctaagtatttgcagacctatataagtgacctacctataacttgctttttaaggatgatatatgtaattagataatttttgcaagcagctggtcttgggagttaggaaaaacagaacaatataatcatccttattattaatatttcatatcaaaattttttagctttgaggctactcaggatttttgggcagataatactatgcttaagtaaattttaaactaacagaaagcgatcaaactgattcattgttcatagttggtatcagtaagttggagaagaaatagaaagagttcagagctcagtgtatttaaacctggaattaggtcattcttttttatctagaaaatcccagcccatgtggccttaaaaatataattttatttttaagaaaaaaatagtttttttgaagactgcttaaaatatatttgtcatttaataataactgtctctctaaatggaaaagtagccagagttcctggaaatcagcagttagttgctgtggattagctgctgcttaaagtttcctaatctgtaatgtaaatgggtaaaatcaggcaaatcaatttctaagaacttcataactttgtgatatcaaaaacaagcaaacaaatacaataatttttcttaaaatgtgagatggaaagaccttcaagaagacctagagtcatttgttggttcaagtttgcccagacgttttgataatgacaggtctcctctcctgacctgtggtacaatggtcaatctccacactctggtcaatatctatcaataacataaaaatgtgactgttcttggatcctgtatttattccccaatattaggcctgagagtgccagaactggcacttaatgtattgatggaacaccaaggagagaataaaaatataccgaagagcactcgacacaaaaaggatacaagaatttactgaatagtgaacaaattatatgcatgtactgaggttcagtttttctaacctgtcaaaaaacaaaaaaaatgacaaccaaacaagaaaaatgtctctaatatagacactaagcagcattattttaatacatacatatatgggtacattcttttttgctcattaagaaccacaggggctgacgcagtggctcacgcctgtaatcccagcactttgggaggccgaggcgggcagatgatgaggtcaggagatcgagaccatcctggctaacacagtgaaaccccgtctctactaaaaaaacaaaaaattagccaggcgtggtggcaggcgcctgtagtcccagctacttgggaggctgaggcaggagaatggcgtgaacctggaaggcggagcttgcagtgagccgagattgtgccactgcactccagcctgggtgacaaagcaagactctgtctcaaaaaaaaaaaaaaaaaacccacaaaatatatatattttatacatatatatatattttttctctctcctctgtgtgtgtatgtatgctctctctgtgtgtgtgtgtgtgtgtgtgtgtgtgtgtgtgtgtgtgtgtgtgtgtgagagagagagagagagattcagattgagatttaaagcccggggctaggtagttttattctaaacaaatatcaaataaagaattgaactcaaagtgaaatactgtatccgcataaaatgtgagccagagaactactgcctaaaaaatgctgcctcacttggaacctaaagctaatatcattcatttatttagttagctttatagtatgttggttttggatttgaaatgattagaataaatgcagatggaaagtgtttttacagtagggaaataattaaattgggtgtggagactttgacaagagaattcttgaagtgacatatgtgtatcagcaaattctttttcttgcttggccttaattcttttgggaagaaatgcttgggctgcagatgtaatggtatattgggaaaaagaatttacaaacccatgcagtataagtacggcaccaaactatcttctttgaaatagcattacatgtatatagtataagccagatgtcaggaatagtattttttccctgtttcctaaagctactaaacttgatgtcatgtggaaagggatgtgatgtgctttgtgtaaaatttgacattccttaaatgtttttctttttttgctttcctctgtgttgctaaccatgtccaggatgtactattgttcctaaactaatgttaaccataaaatgaatttatgagaaaaacaaaattaatatccttaccctaaaattaagtttgttactagatagaccatcttaaaacataaactgaaatgatattttgcattgatgtttttttctgtcttcatggtgaatttatttgtgtcaatttgggtcccatttgactcatcaccgaaaatacatggttcttgtttaatgtaataaatagctcctggacaaatgaattttacagcagaagtttccatgaatcttatttttttaactcctctactttcagtcctttttatttctgataatgaggtgaggaaataatttatttcattcttgtattgcatactttacaaatatcttagggtgtctctgtgttaaagtagtccctataacacacaaatgatttctctgttgaatttctatttctgtttctctttgcttttcctctcttgggccactgactagcattttcaccaagaagagatcaaagacataggaaggaaataaaactcaaaataggaaatttttattttatactcgttttgttaactagagagtgaatagaaatagaaattgtggtgaaattgggctgtgaagtgacttatcaggagttaagccagctcttgtcctttagtagctttgtttcatcctggccacagtgtcaccagtagaatatagtaaggagatctaaggagaatcttaaaatataggaagaacttgtggtatgtatgagctaagatgatagtttttccacatggagaataacaaaagattctgtggatagtggggaggattgactcttctccacttcgtaagttaaagaaattgagtttgctgagaacttaatgattacctgaaattagtaaaggccttggcctgtgtcttcttgccctagtctttactaactttccaattattaagagaaaggcaaaatgactttccgttttttaaagaagcattggaattagtttcagtttaatatgtaatatttaagtgagcctcagggagtttatctattcaatgtctacatgcaacagcttaaaaaaatgaaattcctaactcctattatgtagacagttgtgatttaataatgtttaatgtattaaattatacatagttatttgctagtaaaataattattgcagtacttccggaagactgaaatgtttatctgcatataattgcctgtctcaaaattaaatgctgtattgttttcaaagcagggaaagctgagggctcctttgtgaaacatgcatagcatttcactgtgtcaaatcagaggtccatctagcccaatattctgtctccaagaagtaccaaaaatcattactgccagtttatgtttttctcaaacctggaaggttggaatatagttctaaataccttaaattctctcaataacatgatctggttgtacagaccacagaattgggtaaccatttcttggacttaattatattatcatatattatctttttgactaatgaacccaggaaattactaatcacaatgcaaagtaataattaattcctttaattattaaaaaacacctctttcaaattataaggcatacttcccctgaaaatgatagatattctaatagttaaatattctaagagtgtgatttaattctcatgtcatttatttaacctgagtcatattatagcttaatctttttcttcccaatttggaaagtgttttgtcttgtttcaaagtctggcctcatgtggatgcttctctagtcttttcatcatttttggctggcatttactggagattttcagaatttgttttaccttttctggggtgtgctgacagattcctgcagatatggatgcagatggggctgtgaatgagggtgaatgatgtcctctgttttgctgttaattgtgtctttgatagctgacctatcttgatcctttgttcatggtggcatctgtgctgatttcctcagataattgtctgtggtgtttgccaaatcacttatttcagtgataattgcagctttaaaaaacagtctcggccgggcacagtggctcaccctataaacccagcactttgggaggctgaggcgggtggatcatctgaggttgggagttcgagaccagcctggccaacatggtgagaccccgtctctactaaaaatacaaaaattagtagggcatagtggtgggcacctgtaatcccagctacttgggaggctgaggcacgagaatcgcttgaacctggggggcggaggttgcagtgagccgtaatcatgccactgcactccagcctgggcaacagagtgagactcggtctcaaaaaaaaaaaaaaagtctcaaaacatagactattgtttcctaaatgtcttatcacatatgtgcccaatcgacccttccagtagctcattgggaactcctggtaacttatctttattggccagtgtagctgagtgttatttttgtacactttttgatttctttctgcacttcccctgtagagttcatcattttctaagattaaatgaatgggcaaatgagaataatatagtacatgtcccttctcattgttactcagagaatagttgcttattatgtgtttaaattgaagaagactcatcctaaaacccattaaggaaaaggaataggctacaaccaaaaggaggaagggcctcacagtggcatttcttcattcataccgtgtttatttaacctttgcctcactgtcttcagctccctaggttttattcttgatgcttttgcttttctttcatttcatactttttcatgtataatctctgtttacaactcttgatgtggcccacagtgcctagcatgctccgcccacgtccttctgcttctgctcggtgtgcatgagctgctgcttgttaggaggtctgcattcaccactgctacttgcctctctccacagaggctgggactctccttccctttcagtttctcctttcccctcccacctttgcttacctacatcatttcaaatcatcttccagatgttttgcgcccgttgcagcactctccccttctctaattagagacaacttaatggttattcatttcactagtatagagacttagagcttgatttcataggtcaggcatctacaaactgtgatatgaaaggagagattgctagggacagacctttaagtggagtggctgccaaactcctgaagaagctcagtgctttaaggaagccttccctatccagccaactagtccagccccactgtcataaggccacagggtaccatgttcttctccttcagagattctgcatgtggtagatgcctcctcagttcaccagactataagctctacaaggttagagttcatatctgcctttgttcaccattgcctcattagtatacagcacagggcctagcacatagcaggtccccagtacatatctgttaaatgaatgaataacttccaaatacacaaatctggcttgcaattgctcctggaatatctcttttaaccacaaatccctagtgtaatttagttagggatttctctctgccatctcagctccacttgtgaaaaattgtaattacacttctttccccagggtctttcctttctagtgataaataattctctcagttgtcctgtctcagaacttttaagcaatctttgacttcttgctccctttcctaatcctgctggtcatcaagttgacattcagctatagcccccccaattttctatgaaaccatgttttttgaattttcctctgccgtgctttttgaatctctttatcctgcctcacctcactgccactatgaaagttcagaatcttgccacctcacctctgaatgaatgcagtaggtcttcacatggcttacagggtctagaccctgcctttctgttctgccaagactgtcagagagcctttcttaatgacactttccatatggtgttgccgtgttggtcaattgatcaggcagtattcagtagacagagtatatacagaagcatgagcaaataaccttaaaagtttatatgtgcaggaagtaaaaatgtgaaaatatactttttgtgcctaattactgcccacaaaaggtcaggggttttctgggctacctgttgtccactctcctagttctcttatttttatttttattttttgagacagactttcgctgtgtcacccagactggagtgcggtggcagaatctcggctcactgtatcctccacctcctaggtttgagcgattctcctgcctcagcctcttgagtagctgggactacaggcgcccccgccaccacacccgcctactttttgtatctttagttgaggtggggttttgtcatcctggccaggctggtctcaaactcctgacctcaagtcatctgcccgccttggcctcctaaagtgctgggattacaggcgtgagccactgcacctggccctatttcccttattttactctgttaaatgttgcttgagtctcattttgtattctacctatcgttccaaactgacatagcagtgcatggctctaacattagtcccaaaacattctaggaaaaaatatataccagttacataggtaatgttttccatctcccctgctgtctccatttcctacatgtatcatagagtaaaatcatgtatgtgtgaactctgtggccacccaagggcatctatttttaggtaatacttatctggtaagttctacagatgaaatcatttcattagagggatagaggagaataagagcaccagagtgcagtttgagcactgggggttagtgtggttaggaagggcttcatagaagattggggggctatagctgaatgtcaaagaatagataaattttgaagaaatgaaggaaagagaactcacagtgaggtacaaacaaacaaacaaacaaaccaaaaaacccaggagctgaaattgaaaggtatggtttttggatagtcccacttgatccatgtgaagaaatagtagatgagattagaaagttagggtaggttagcacagcttgtggctggtttagcttgctagttgttttagttaggactcttattggttgcagctactgttgtcagccagttgaagcagaaaaaggaagttttagtatctattattttttgggttttgtttttgttttttttttttttttgagatggagtttcgctcttgttgcccaggctggagggctggagtgcaatggcatgatcttggctcaccacaacctccacctcccaggttcaagcgattctcctgcttcagcctcctgagtagctgggattacaggcatgcacctaagcaaaggattaatttcttaactcgggaaaagagggcttgctgaatttcagaaggtatgggagacacagattatctccctgttcatctcttattcatctctgtcccctttgcttcaccgttctgtgtctttatctctctggctgaccagctttgtttttctccaggccacacaacaagaggaaaatgcccatgtgacactgctcctgattttatacatttgacttctttagccacctagataatagattcctctctttttcattatctcagttccagatttctagggacgggaatgttttcagagagtcagggtcacaaaacagaaatgtggctttcaggagccacctctgggagcctatgaattgggttgtgaggagctttcaagcaaaggttgagggtagataacccacaagtgccctctactttctactccttcccttcccaggacatttttaacctgccagttttatccagcacctacccctccctcatccttcactttataatccagcctatataaactagcatatatcaaggaaattgaaaatgcactcccccctttcctaattagagacaactcaatggctattcattttactactatagagacttagagcttgatttcataggtcaggcatctacaaaccgtgatctgaaagtagagattgctggagacagacctttaagtggagtggcagcgaaactgcaaagtaagaggaactcagttcccctctttatgtaaactcttgtaccaatttttggcctccctatacccatccccaagctgaccagttcctattcaagggacccagtcttctcttcggctctcaaataccaattcaaatgcaaattgctttctccatttcctgtgtgtagttgttagataattgtctgactaatgccctcaattttgccaagattagtgtctaggttttagctctttaaaaaaaaaaacaatgttcaaacatacctgcagaaagacagtcagcaatcaataacctagagcccagatttaaaacttggcaagctctcttacaggtattgagagctcaccaatggatttctgatcaggtaatgatataattaaaattgtactggtgatgacagatcaaccgtgactcaggtgcttggaggcagggagaccagctatgaaacttacataaaaggctggagagacacttagcaaattgtggttattttggggcggcattacacactatttttttcatagcttaaacacattttccaaattttctacagtggatacttatttcttttgatttaagaacaaatacatgcatataaagcctgaaataatagatttggcttgaaataggcatggtggcagggggaaggatctggcctcttctttctgacagcttgcattgttctttagagatgagaaaatgcctgtcttcgccacatgtattagtgattaattccagaaaatggctgtgttcctggtcagcctctcctacagtaaactgagctcagaaatctttgtggttaacagtacaaaactcgttctggagactgttagctgcttgagttaatttggtcagagtaattcctcatgtaaagcagcatttgtgcagtgacttctatccagaagacctaaggagctaggacaagactctgctttgcatcacaggggtaattagaatgttgtcgattgagggcaacaaccatctctgagtattctccaagattgcttaggggttattaagcatcttaaatccttattttgcacttagagcatctgtagtgcagaaattctagactcagtgtgagtagaaacatagaagcttttgtggtccacttcatgcttggccaactctagatggtaatagaaagggggatatagtggttttcctctcagtagttcaattaaatgcttattctcccccttaaaggtactatttattgagcatctactatgggtcaggcaccaaatgcttgagatacgtcagtaaacaaaacaagagcatacatggtaaaggagtatgttagaaggtaatacataaccaccaattaaataaaatagggaggatcaggaacattgcggaggggaatggttgtggttttaaattgggtgatattttagcaaacttgaaggaaatgaatgagtgagccaagcagagagctggtggaagtacactcccttcagtaaaagcctgcgtggcatgttcaggtgacagaagagggaaggacagtgtggctagaatagagtgagcccagagttcaaaatttgaaatagcaatattctagaaaaagctactatttctagtcttttccacattttggaaatatttacttgtaatttcaataaaccattttcaaataaatgtcttggactaattgcctcttttgtcactgctacaagatggggctgatccccaaccaggaacacctctctctgccacaccacacacatcttctaagactagggctctctcctgatcagaaggcttcaagattcttcaccagtggaactcgaagtcttaaggctttttctaaagtagtgttcttaaagtttcagtcttaaatatagatgagggcttaatagagattccaaaattttgatttgtaaaggtacagagattctgcttattatcttacatttaatcatcacatttcagttgtcttagcaacctacacagtattctcatttggttttcaataagccaatggatgaatgggttctttgttcacgtgtttatgaatttctatcacctgatacttgaagtctttcaggcatgcttactgtgccaaataactgtctttatattcctaaaattcatgtagctaaaattaagctctactttttctaaatatttacatttccaatgagagaatctccaacataagaatagttggaaaaatgtatatataagcttgtgttttttttttttttttttttttttttttttttttggtcatcgaatgatttaatccactcttaaaattctagaaaaagttatatcaaacaaatacttgtcttgcttgggtagtaagagccaaataatacaggtatctttagttcaacagcggcacctttataagcttgtatttaaaaaatacttttgctttagttataattaatttccaaggctccacagacttgatggcaaactcaattgtctagcaaacctcacccatacagacatatttgagaattacagagagaaaaaaaaaagcctatgacaaatctaggcttcatagaggtatctctgagatgttgtctcagaatgttttgtcttactgaatgcacagttttaaattctaagtgaaggaatatatcgaaaatgtttaacaaagtacctgccactgtattatgtgaatttaaatgactgctcttattacaagtaggcatagtgatagtattgacaagtgacagtctaatgacaaaaagacaaaaatagcgaagcataaatgtaagaatgcaaaatgcttagtcacatgagatattttggcatgaacactggaagcccagtatgctttagtaatccctgaggacattgatatgccttagtactgtgtgatgattctcactagtgttggtcaccctgattttctcagtaaaagttaaattacatttaacatcataacattttagaattatttatatacaaaatgatcccattaaaatattttagaattatttatttacaaaatgatcccatttaaaatatgctattgcttaaaataaccacagctcagcaacacacaatatttgactaaaccataatgaccaaattccttgaaggttctggatagatgaggttatctagaattattcctttttcatgctaacatgagcaactcaagcatctggatcacttagggatttttttttttttttctccaatccaggtcaggtacctagcacagtgtgtggcacatagtaggagtgcaataaatatttataataggaattggtgaatgaatgaatgaggagctagtttttatattttggggagagaagtggcaagttcttcaagtctttacctacactagtcgcactaccgtatgccatataagaaggtttaaatgtcatctgtaaataagatcaaatctaaggaagacctctaagtttgggctgtggtggctcccgcctgtaatcccagcactttgggaggccaaggcaggcagattatttgaattcaggagtttgagaccagcctggccaacatggtgaaaccccatctctactaaaaatacaaaaaattagccaggcgtggtgttgcgtgcctgtagtccctggtactcagcaggctgagacagaagaatcgcttgaacccgagaggtggaggttgcagtgagccaagattgtgccattgcactccagcctgggtgacagagcaagacttcgtctaaaaaattaaaataaaataaaaaccctctaagtttgctctaacaagtagagtaactctgtatcccagcttaccccatgtagagtcccagtttacacttggtatctcagcatcccttttggatagagagtttactttcaagaaagtcttgtttgcatgatgaatttaatggttattctaattaaagtcatcacgtccttaccaaaggtttttgaaataaaaatgtaataatcaagaatttccctttttctgtctcccccttcttttctcccttccccttctttcctatcttaggatgacttttaccatgggcattgcacttttatctttaatacaagacaatatgtgaaaaagctctttgaaaggaaagatgacttgggaaataattgttccatttttttaggcttggcttccataggaagctcttctaatgcattctgtcatgtgattaatttgtcaaaattccatacctgtcagaaataatgtaatcttttcaagaacatagacagtcttattacatgaagtgatgtaatcctatactatttatctcctctgtattcctcattctcttattttctattattcttcaaatagaagagctaaagtcccaactaatggggatgttagagatcatgctagagcctgggaaaaaatgattaatgtggtttggtgggatgccaagacagatgcgccacaccctgctcggtgcaacagctgttgtagtaaagactgaaggcattacataggagcatcatgtgtgacatcgaacgggagacaaaatagtttactgtggctgagacactgacatttacatattcttaaccattatgttatgtgtgtgctgttggggtagttattatcctaggtttttctcattatttataggtgtttgttttaaagagaaacaatgatgaaaaccatggtatagagaaaaaatgaggagagggtgtgtttgtgtgtgtgtctgtgtgtatatgtgcatgcatgcaacatgactggaagtgtggattacccattggtatttctccaaaggggatttagaagggttaattctttctgcgcagggctgacagcacatgacacaactatccaaccatgccttcttccagatctcagtagcagttccagtcacagtgagaaccccaaactccctgccgtgttttcacactctcctggctacccaagagatgcatgattttcatcagaggaagatactgtgtatcctaaaaattcacatctcatggacttaaaaagatctggaaaatgcaaggcttagaaaatgttagtgaagcagacttttaatggcccagaatgaatcactcaacttggctggaaaaagcattctcttctatagcttctgacaatgaaccatggcatggctgcccagatacttaacattgcaatgagggcatccacatgacagtataggcagcaaagcacaacccctcaataccttagaaaagcaactcacagttggtttcccagtaacatcatttgactgtaggtgctgcttagaaagagtcaataagctccagatcctgaattcaaagccttttcctgacactggaagaagagcagtgtgaggcttcaaattgtttccaaaacgcctacatttccagcatcttttctctttccattccatttccaatcagctgcattccatctagcaaattcttgatattagggcatacagatttttagaaaattgaattttatttcatggaaaggttatttttttgtgcagttagaattagtgatctttgaggaatattgggaatagttggcttttcacatattgataactgcttcttgatttattgtttatcagtgctctaaacagggtttggaaaatatttggaactgattccttttatttgcatatatgatatatccccatgtgtattagttttcacactgctgataaagacatactcaagactgggcaatttacaaaataaagaggtttaatggacttacagctccacgtgcctggggaggcctcacaattatggcggaaggcaaaaggcatgtgtcacatgggggccgacaagagaagagggcttgtgctttttaaaaccatcagatctcgtgagacttattcactatcacaagaacagcatgggaaagacctgcccccatgattcaattacctcccactgggtctttcccacaacatgtgggaattcaagataagatttggatggggacacagacaaaccatatcaccatgtttcttacagtgcattgttttgtgtgtcttttcccctatggccatacattgtgaatccatttagttaaagcaactgagtggccattaaatgttttgtgctaaatgtaagagataaaactggaaaaaagaaaagggggggaggacagtgcaagatatcagcccttgttgcctagtcaagaaacagaggaatcgtgaaagaaagcttattttccatgtttgcaattcctgggaatgaccaaactggttttgcaaaccatgtgaattgagtccatgtgtatgctaggggtgagggagttattgagactagtttatttgagatattctgggtgtcttttactttctaaatattcttactccttttcttatgggggaaagaattttatgcaatagactttcattaaattgaattttcacaaccaaattgaagctgttatcagttggttgaactcttgctaactgaatgactataaggacaggtttcaatttcttcttatttaaagagtctgatttttttcagctgctattttttttcctcatattttcatgttgcctttatccagttggctgtttacattgtcagtaataagagttagaacttctaagtgtgacatgtgttttttatttcaaggacttctactaaaattgtttcttttgcatgcatgctatggaaggcttgcttaaaaactttgagaagactctaagtagagcccagaatttcgctaagaatgtggaccttttctttggtaagttggccatcctgtagaaagcgaatggcagtttcctaaagaaaaccctaatatggcttctttgaaaaaaaattgccaacatgcagtgccaaatcgtatttagtaaaaccttgtccccaaataaaaccagggaaataggcacaaagaagagaaacaggatacatcagtgcatgttgcaggagggttggatttgttgaaagattaatcataaagtatgtggacaaaagtgaattaagtaagtataagatacaatgtcactacctatttattcatcttttagctaaatatatgccttcccatgtctgcggacttttcctctcttattttgaccagcgccagctcacctctgctggtgcccacactggatcatgctaaattcaggtcagcatgcaaagggaatcagcctgcttagctagtggattttctaggcataatttatacatacataatttttagtgtcaggccaagcattttcatttttataatttcttctttctgatagtaaaccatttaatttggagcaaatataaatgattcctaatgaagcacaagtgaacagaaggaaattacttttgttaaactagtacttcaatgtatttttctctccctttcaagtagaggatgactatgagaaactgatacacagtattattcattatctggttagttcactagatcaagatgttgatgtgtggtgtagaaaggtggaaaaagttagagagttaaaaacaggttgatacaagctgttgagaaaaaaaaatacaatgaggctacttatgttttaaatcaagaatatgttttcagtgaaaatactctctaatgattcacagaagaatgtcaacttggactacatctgctgaaaatcaaagtgtaagtagttatatggcattgaattctggcaggagtctatgctgtgatatgaaacgaaagttcccatcaaatgacaatactttatttttctgcctttaagagtagttttctgactttaaaaaatgttaacagcattttttctccttcacattacaaagataaattggcaacgtgcctgtatgatttccagagtaggtttcttaaaataattttgatgggaaggtaattgtttaatgtcagtgtataaagttctcccatagtttaaacaaaattaaataagagaaaacacaaagaggaggttttgtataattactgggaaaacatagaaggaacaattaggtagttagacctgaaaagaagtgaactagggttttcaaatttcatactatttatagggttccttgcattattctctgacatcacttgatttttttgtttctgagtttgcaaatcagaatagcagcatatttatccattaatacatatttatagctttttgcacttatatattttctattcaagagtttcatgataaaatgaaatacactgaatacatattatgtgcaaaacaacaccattacaaaagtatacactgatacagatgcgagttagaaaattatatgcggtggggcgtggtggctcatgcctgtaatcccagcacttctggaggccaaggtgggcggatcaagaggtcaggagttcgagaccagcctggccaacatggtgaaaccctgtctctactgaaaatacaaaaattagtagggtgtggtggcgcacgcctgtaatcccagctactccggaggatgaggcaagagaattgcttgaacccgggaggcggagggtgcagtgagctgagattgcaccactgaactctagcttgggtgacaaagcaagactctgtcttgaaaaaaaaaaagaaaaaaaaaattatatgcttatattgcaagccgctttgcataaataatctgttctaatataggattcatttagcaaaattcatgaaaatagacatagtgtttgtgccctgaacatcatattgaagggtatcccagtcaattaaaggatagtaataaagtataaaaattattattgattaccttatttttttacagtgttatttttaaataccttggagatatcttaaattgttatttagaatgtacaatttaacataaatttaactgaagttgggacctgcttttttatgttaaattgtagactttttcttactcaattgccataaattctaataaattctaacttttttgcataacagtggagagaaagtttgaaatggttcatatgagcagctttgattttggaatgagactggggacacagctcaaggtttcctattttctcctgtccctctcttctggaatcaacccaagtgcccatagtgaatgaatcggtaaagaaaatgtggtatatgtatacaatggaatattattcagccataaaaagaattaaatcctgttatttgcaacaacatggatagaactgggggtcattatttaagtgaagtaagacaagcacggaaaagcaagtatcacatattctcactcctatgtaggagctaaaaaagtggatctcataaggatagagagtagattgtagattggtgtttaccagaggccaggaagggtaggaaggaaggagggatccaaagattttggttaatgggtacaaatataaattagatagaagaaataagagctggtgttcaacacgtcagtagggtgagtatagttaatattaatctattgtacatttcaaaatctctaaaagagaataattggaatgttcctagcataaagaaaatacaaatatttaagatgatggatatcccaaataccctgatttgattatatgaatgtatcaaattatcacacgtaccctcaaaatgtgtccttctgttacgtaccaactttaaaaagaaaataacatcatataacaactatagcccttagatgcaaaaagacctgagtgcaaatatcattccctttcttactaacatctaagtttcatatagtcttttagcttcatcgtttaaccccgaaatctggtaccatcctcttctattttcttgatgactaaattagatggcattatgtgtactgtttgacacatagttaacactcaatgaaagatagttatcttcagcctttgtacaaataactctagagaaatattagttgacctattgaaatgtggcacaatccagggctcacccctctcaggatcattagcattagattgagggtatttccctgcatacctctctctaataccagaaactcaatattttagtagtcacagggaaaagatgatagcaacagtgccatgggtgaactgtactctttttctgttctttgagcataaccataatgaatcaagaggaaaaggggaaggaactgaggagttgcccaccagctgtttctactgactgtgggaggaatttcctttgctcttttgttctaaaagatatctagatcatttcagcaaaaacccatatttttaaatcaaaaaaaagatctatatgattctagggacacagacacacacacacatacacacacacataccccttctttacaccattgtagaattgtaaaattaacacgtacagctaccaaagattgcatcatgacacagaaggatttctaatggcagtacttaatacctctccaaaataccttaagagggaaaaccactttggtacatctgttgtcactgagtggtgagcacttcttatattatctttattctctaagtagttgctcttcgttatttattcattggctctctaaatacagcacttaccctttttattccctgtctgatttctaaaaatggaacattcataaaataactcagtattatataaaaatttctaaagtgagaatctagctatatatctggaggcttaaactcacattgttttgttaccaacacaaatgttgaaatctactgaaagagtgtagtcaacttttccctgagcaaattcattttgatgaggcactgtgggaatttttgtctggaactgggtgtcatatgcttacatctctgtctaggctactgattcagggagctgtgcaatataaagatgaaatgtgatacacacacacacacacacacagccaggagaaataaaatgtgaacccaaaagaataacagaatgaatctatttgcaaatgtagtaaattcccatgtttatagagaaacaagagggagggtaacaggggacaagaccagtaagaattagggaagcacatggcttatagaattgttcattatctgatgtttccatgaattgtgacactctaggagtggagtctcagcccctccagcacccactctaccaacttgggcccttcagtgcctcggttttatcatggggctaataacaatgcctgctttctacaattgttatgaggattaaattagctataactaaaacacttccagcagcatcaagtgctatttttattatttccattactaattttgtgtttatgcattttgtggaaaaggaaacatgaacattgtgtggttcaaatagccaaaaaaaaattaaaatgtgattacactaaattagaatttatgtgattttattgcacctgaaggttaacatgattctgggattctaataacaaaaaagctttaaaaccatcttgatttgtttatacagaaaaacaggaattggcctgatagagaatgaaaactatgaaatagctggctgtcttgctttattgatatttttcacaggctttatttaactcccaaagacctagcctaaacaatagtatttttagaggacatagtcacataaataatctttctcattttgctctaaatgcctgctttaaccacagggggaaaaaatacaactttgttcttgtatgtttaagtcaaagatagtacacatagtatataatgttaattccatattaaaatgaccatttgaagaaagtgttcattaagaaagattacatttacttcaaggaattttcttgatatcaagcaatatttatttttcataattgagcttatgtgcctaaaataatccactcaaatctggataaatcatagtcttctatttgcacattcatcaactaaactctcatataaggaaggaagccactctacaagtcatatttctgaactgggaattatcttttcctgtcatgttttttgtttttgtttgtttgtgtgtttttctttgagatggagtttcgcctttgttacccaggctggagtgcaatggcatgattttggctcaccacaacctctgcccccagagttcaagcaattctcctgcctcagcctcacgagtacgtgggattacagttgtgcgtgtgccaccacacctgggtaattttgtagttttagtagagatggggtttctccatgttggtcaggctggtctcaaactcccgacctcaggtgatccacccgcctcagcctcccaaagtgctaagattacaggcgtgagccactgtgcccggcctcctgtcatgtatttttaacaggagtgtctcaggaatttaagccctttatttggactgactcccctgttcacttatttttctcagttcagtgagtgttgccaataagagagagattagacaaaaaagacaaagcacatcatctcttttctaatgtggatgttactcagtgatgaggggagagcagataagatccccagttaccaaagcgattaataacatcattctgtccattcacactcattcagccagtgtttattgaagagctttgtttgtctctgtcactgtgatcctacaggaaagacaatacctcctcttaaccaaggttgacaagtagctactcctagtttaataatttttgtaaaactttcagagctgttttttttttcgtattacaaatttactacattggatctactacactgtagattcttcagacagaaaatacatatggtcatgctaaataaaacaaaaataatctctagtttcagcacccacagtcttctagtcatagagagcaattccagttgttggcttgaatagtaaagcttttttctaacacaaaacatttgagactagtaacttcctatgcatttagttatcagttaatgtaagtctgtgtgtaataaatacgactttcattgagtttcgggatgtccttcgtgttcacgtgtagccctgcctcatcacccaccttaaagtggattgcttgtatccagtttctagtctccttggctcttcgcatgaggagagagagaaatgggaaaccctaattccctgttcacttgattcaacatctacgatttcattctattttaaaactctaccctggcagtaccctgcttactcattcgtagctgccttcagacatgctattctctgcttgaacatagtcgctcttagttcctcaacttttattttctcacttaagagcctgcttcactgagaatcatttcctagtgtcatgattctgaggtgccttttccatccactccttcaggctcctgtgctcaccatgccatggcctcaacacactgtgctagaatattgttttaccccctgactccctaactggactttgagttggtaggtagcattttatctgtcttccaaggatcttagccatctcctggcacatggaagtggtcataaaatatctattggatatatgaatgaataaatggactgacatcttgtggcaacatcctttcccccagtaaaaaccagaatggaatatacttcttatagtccagtggttctgactgaataacatatttattttggtagcagaaaatgccattgtttgggaaaactcaaggccatctactaacttctttattcttgcattgtcgtgatgatgtgactgatttccacagatctcataaaaatcattctcggtattctatattctatttcctatgtatgaaaggtgaatactcttgagtcattaattcaacaaattaattcaacagttatttattgaatgtcaattatgttccaggcacaattttaggtggggctacagcaaagtataaaacagacaaaagtttctcctttggggtttacattccagtggagagaggcaagcaataataaagattaatatacaaaacagtatattagaggtggtaagtgcaatggagaaaaagcaggaaaagaagagagagaatgaatggggggaagggggtgacaattttgcattgcctggccagggaaggccttagtgagagctgggtctcactaaggaggaaatgagggctgggtcatgcagctgtctgggagaagagtcccaggaagagagaacagcatgtgcaaaggtcctgaggtgagagcatgtctgttgtattttaggaactgtgtagtcaactggagagcagagagaatgctgctaaattctattatctatctatctacctacctacctacctacctatcttctatctatctatttagtaacattgtatcagattactttatatatatattgtataggctgtcaaacttcattcatatttttatttactaattgaccaggcagttatttaccacctaccatgggccaggttctatcttaggtattttaaggaaagtaaaatctattagatatagttcttgcccaaaagaagtttacgaaataatcagagataagacacatacaggactcatgacacgagatgagctctcataaggaaatgactactcattggccagagaaacagctagagaatgacatctgggcttcttggaagagatggcatcaaactgcaccttgagaggcatacaatgccaacggcacatcatatgcaatggtgtaaacgggggcacacccactgagtcactggcagtgcaggatctgtgaaggcaggagtgataggtaaggtcaaaaggaaagctttggtcagcatttgaaagcctttgaatgagtatatattttgtgaattctcttcattaaaaatgttctaagatttgtcatgatgttcagtgccttggtcttcataattctgtgttgccctcacctaattgccagcatattttagttaaattcaattagatatttcagttagctatatacatttttcagattgaaagctgttaacaggttgtataaaacctgtttcttatactttaacaaaataatttccaagtgtactatgcgatttttaattctagaaggttttctattaaaatgaaaatgaaatgtatgaaatctgtctcttattatttcagtcttctataggttttcgagtgtaaattagggagaaatgcagaataatttaaaattaaagttacttatagagaaagctgaatagaaaaataatgtgtatatagcatttgtttttctagaataaagtaagcacttatttagcttttaggtttcaactatattgtctttctcttgagcagattttacatagagctggtatgacatatctcagtgtaatacataatatattactaatgtctttaaattcagaagataagaaaaaactttgagatcaagtttttaatttgggaaaggtctaattaaactccttacattgggatatttgagttaattgttaaagtgctaattcagtttatcaaccacttgttatcacatagttttatcaacctacactgtgaaataaaggagtctagggttttttttttttttttttggaaggaaacgtatttatcgaaagagatttatttagattattctacatattctctgggaatttgtagtgtttcttaaagttggactaaatgttgaatgggaggataggaggagaaggcgatggaccctacaggtgcaaactctttccaaatcaccagggctttggctgttactcactcacccccgccttccttgctcttggagccttcagatttttccccagtagcttcaccttacagcaagcttagcagaagcagttccacagcatgccaacccccctcacttttgaataatccagagactccctgtgccacagataacatgcaatcataaatcttctcagcttcagcagttttcgtcttctaaattcaattaagttgagcccagtaagaatttgtggatcaaacagtgagctctatatagattcaaattcaacttagggttctgatgtttgttactaattttcaggaaaaaaaaataaaagaaaaatactgctgctattttctaaagcttccttataaaggtatttcttattgaaaccaactcatatttctcaaattcctagaccactatctgtcatgcaatttgagtctgttttccaaatggtagagtgccagagaatcaaaagctgcttgttttgcttgcctgattttctttagcattctttggaggatttaaagtatggggggaagaaaaggaaacctgacttacattaagagctgaacatgaaaaagggattaggcaaagctttaactttttcattatgcaaactttcctgaactataatacatatctttatagagttttattaattgttggggctgcaatttggattttgctaaaaagtatttactactaaaaacttcaaggttgttactaaacacatcaatcattttacatttggcccctttgcatggtgaacagaccgtccttcctccatcttttgttggggaatacctggctgaaggtctcagtgaaaagctaggaagatactcctcctctgagggagacatctggcttcttggctgattccatgcacacttttgttaatgggctcagattccccagaattcttttatgtggttacattgtagatggactgtctgcatagtcttgtcctcttgaatgatttatgtagaggttaaaaatacatatatatttggtctggttcttagaatctacgcataagattaaatgaatctcagcaagttcagttttggaaaaaaaaattaaatttattattacaaaggcaatctagtagaggatgtagattctcgagccagaatgcttaatttgaaacatggatttgccacttgctagctatacagtgttggacaagttatttaatctctttgtgccttcttggtttccctaaccacatacaggatgataataacaatatctacctcatatgtttccatgaggtttaaatggtttacactgtcaaagcttttaggacagtgcctggcgcgactaagtactcagtgagtgttagtcatgatgtcatgattattcatggtattcaccagacaagaatatgttctcatttgtgtatgatttaggattaaatttgggactaatattgagtggttcaatgctaattttgataaggctcatggaggtgtgtggtcacttttagtggagagtaactttaattcctttttacatagtgaaaaaaagagttcaccttccactcactagacttgattttgcagactccctgttctgatctggattttattatgagtgaaatgaagttagtgactcagtggtccataggtcattagtctgtagctgctctgttacatggaaaagtcctgaatgacaaaggtctactcaagggtgggttggcaaggtaatttagaaacctgtattattcccaaccaaaattggagcataaaaccttctattcactatccaaaaaggaagtgaaatcagccccacatagagatgtatgcactcccatgtttattgcagcgttattcacaataaccaagatatggaaaacagttgaagtgttcatagatggttgaatggataaagaaattgcaaaatatatacacacaaaaaaagaagtgttattcagccttaaaaaggagattatgccatttgtgacaacatggatgaacctggaggacattatgctaggtgaatatggcagacgcaaaaagaaaaatactgcatgatctcacttatatgtggaatacacgtactttctatagatagatagaaattgtatataatttctgttcccttcctcagggtaatttaaatggatattttaaaatagagggctattaagctgttgaaacatttcaaatacaactataacctatgtatatctctatatgtggaatctaaaaataaaaaagtcaaatacatagaaacagagggtagaatggtagttaccagagatgggaggagggatcaggaggtgtggatcaaagtttacaaagttacagttatgtacgatgaataactctagaaatctaaagcactggtgtgaagattatagtcaataatactgtaatgtataccagaaatttattaagagtatggatgttagaaaatctttcacaaaaaagaaaggtgactgtgaaagaaatgatatgttgattgtcttatcattttactaagtatatgtgtatgaaaacatatgttgtacaccttaaatatatgtcctccccccatataaaatttctgttcccttcttcagagtcatttaagtggatatttaaaaaatagagttattaagcttttgaaacacttcaaatacgtcagcctatgcatattaaaattttaatgcagcaaatttactaatatttgcaaaaccagtttagatagagataatatatagatatatagtcagattctgaccatgtcctaagtgttcttagtatctattgatgaatagatttcattaatttaatgagatgatagtatactaaaacttgtctaagtaaatggttggcgtccttgaaacttcctattgaatacaacatgagtaaaatgttcgtgtaaagtaaatatttttcaaatagggatgtgttattggtgtttcttaaatatagattacttgctgctgctttttaaattggatataaactttaaagtgaacagcattgtaggaagttgaagaggaagaaaatatattattttgagaaggtattatttaaatctatataaatccaacttaattaaggacattggataattactattaaggtcacaaatttataaaaatttacaaactaacatgaagtttaacagctttattcaagagtaaagttcatgaattgactgaaagagctcctcctaaccaaaaaaaaaaaaacttctgtatttttccatagaactaaggtcttaattccttctatcaatcatgtgttttccccgtttgcattgaaggctccctactctgtcctgcagcagcagtccctcctgttggcagggcttttctctcctcctgggggcttcctgcagtccacttcaaacagggctctggttccctagcgggcctgttaaattattaccctcaggtgttgattcatccttggcattgttgcattttaccattttgtagtcttcaggcatgggggaaggggaagagagttgaagaagatcaaagtagcagtactgaagttggagctttagaggtggggatgagccctcagcccctctgctgttcctccctgcccttcccccgcctgaccccagcctacccccaccaccccctgactttctgccctctcaagcctaatcagaagacagagggggctatgtgcttgaactgtttacttacaagcactttatttttacttttgcatacaagcaacaaacggtttaacactacatgatgttttattgtatagtacaactgtataaacagaagataacagatgaggacttactatttttttgattcggtggtttttgaacatacactctattctctgtaagacactgtgggggaaggttatgatgagcaagagactccttgttcccaggcagtgctgagtgaaatagttaataaatttttatggagcgggacccaattattacttgtgttatagagcacaaatatgaaactccatgagaaaagcacatcaaagggaaacaccagcatggtttttaaatatctgtaatcagtaaagtattgcatgttctttttagaaaatcagaaaatactcaatttattaaactaaaaacacagtcactctacccagagttaaccacactttggcacatttaattttccatatatgtgtgtgtgtgtatctgtatatctgtctatctatatatatatatattcggtctttttttaatttttgtttcataaaatgtatgactcagtctatcttgctttttaaaaactctacattatcttatgaatatttcaccatagaattgaaagttatttgcataatatatcagagcacataataatttatgtatgtaatcattttcttatttggggatatttctgttcctttcagaatttgtcctcttataaataattcattgaatatatttgtgcataaatcttctcatattcctgattttcttagagtgaacttctagaaatgcaatttcaggtcaaaggtcatgcatattttaaagctcctgaacattttgccagatagcattccatcaagcttataccattgaaaagtcaaagccagcatggcagaggagttcttgtctcatgcctgtcagcaccttgttttgtcattgtaaactataattgttgctgttctgttcccttcttcaaagtcatttaaatggatattttaaaaagtagagggttattaagcttctgaaacacttcaaatacaacttcaacctatgcatatctattaaaagtttaatgcagcaaatttactaatatttgcaaaaccagtttagacaaagataatatatagatatatagccagatcatgactttcatgatgtaagtgttggttaagtaaaaattatgagttttttagtttggcagatttttagtttatttttaaaagtacaatatgcaaacagaaaattatacatatcataaatatgcagttttgtgaattttcataaaactggatacacccatgtaatccgtacccagagaagacatcgaatgtgatcaattcccagaatcctccctcatgccctcttcacaatcataccccttcttcctgactttcaacactgtagatcaatattgcctgtgttctactttgtataaatggaatttcgtgtaatgtatgtattttttgtgtgtctggcttcttttgcttaactttgtatttgtaaaatttatgcattttgctatgtgagagcatgtgtttgttatagtctccattcgtttttgttttgtggccctaatgttgagtgagacctcattaagatgtcatgtcttgatacaccttacatggtctctgaatgatctttgcttgtttcctattcagcatagatctcatggctcaccttttcaaccactaatcttccaacagtcttaagttcctgtctccttttctcttcatcttctttacattgtggcctttggctgctggttgtttttgctgtcctcttcatcggacttcctttggtgggtttgaagtggctatttccatactcaccacagcccagcagctggatgtggtaaggacagggagaacaagcctgcacacctctcttacactcccttccattagataggatgcagtcatgtgtacacttacatataagagatattctaaagtacagaattatggttgggggcccaggagcacaacttaaatcctatttattgcaaaggaagaaggaaatcataaaatactggggataactatgaaatgtgctagaaaactctaccaatttgttctttattctcagctaggccattaatcctgcctggaaattattctgtttcacagacaactctcttttatttctaatagtatttatttaaaaacttctccactctccttaaacttactatgtttcatcttgccttcatagttccacatttgaaggtcctgactcccacttcacggataaattaaaagcctgtctattgtgcctcattctgtaaatggccaattctctctatatatattctgggtctcctaccatttcagaggttaccaaaaacttttgtgttaacccagtgattttttttttttttttgggcctttactttcttaacctctgctaaatttatcactattgacgtgttaactacttcttaaaatattctttttttagctttctaagacagtatctttctggctttcctgccatcttttgattctctttttttctgatatcatgtatatgttcctcttttatgcttatcccttaagcaatgttgcttcccagtgttctatcacttgtatctttttacttaccacacttttctggggtgacctcttccatacccatagcttgaatacccagtatatgctggtgaatcttaaatctctgtttccagcatagatctcttctgtgaagcttcctgctgactgctggatatctctacttgcatatcccttgtgcaccttaaactcaacatgtccagaattgaactcacatgcatctgcaaagattctcctctttcttttcttcttatcttggtgcatcgtgccttcttcatcacagaaatctggatgtccatcctgattcttttttcatgctctctcctcgtatttaatcaatcacaattccctagaaaaatgtgtcttcttaaaatttctagaatctattgactttcattgatctctactgttactactccagttcatacccataatctgtcaccttggtagtggagtctcttgatcatttccctgcatgtggttttctcttctaatctatgcatccagacatgtaagacccccatggtgtggtgcctgctgacatttccaccttcttctccgatcctccatgttctctatgctcatctccatgtcttggtttttacatgtgttgcccactctgcctggaatgtcctacatttctttgtctagcttctgtttagatttcgaaaaaaaaggtaacagttttctctgccaacaacctttgtctgactcacacactcccaaggtttcatgaagaatccctcgtatgtattccccaaagcttgccgttatgatagattacttagaaaaatgtattttaattttctactgatttgatgttacctttactagactttgagcttctcattcctcactgtatctatagatgttatttaaaatatttaacacgagatacagcattggcatggatccaatagaaagaagggtggggccctttgctatgccaggctatatctcttcaattcactggacttcacctggcttcctcctttctgcaccatggcctgaaaattttccccagggggtaaactggagcaatcatagggctcaccagatttgttttccatttcccagggagcactgtctcatgctgcctgatgttcaatatcttgagaaacattattatctgtattttatttagttctttttagtttttttcaggtggaaagataagttagttccctgttactccatcttaggtaaaagcagaagttacaccatgtctgttacctgtttgaggtgggtgacgttatatgtcagttatggacttttattgatatacttagagaataacttaaagatgaagggacatcagaaataagcatttctggagcctttattttatgccatattttttatgtacatgaatttatttcatccaatttggtaaatcttattgtcctgttttacagatgggcaaattgtaactaaccctgtaactaaccaaagttcacaaagctagtcacatccagctttcaaactcaggcttggctgctatcaaacctgcctcctttctcacttacccacactgctggtttggtaaattccagagccttattgtattgacaaaggacctcattactgggtaggtaagtgatccactagagtgaaactgtgccttggtcctgaggtgaagcgagtctttttacctatcttcttttttgtaaaaccctccagtagttccccattcctctcagaataaatgcccaagaacttacaatagtctgaaagtccaatgcagcctagccccccatgatatctttggctccacttcctcctcttctctcactgctcggtcttttctgggctgctggcctttgctctgtctgagcctgccagacgtgcctcctctcacagcctttacgcaggctgttctgtttggaattttcttccctctggtatctgcatgaatgaggattatgagacacatgtgccaattatatttaaattgtaccctgcctcctcatcaacactgtcgatctctgcttcaggttttctcctataatttaccatctcctgacataatgcacaatttacttagttattaaagatgtggggttaaggagttccaactcctctgtgcacttttatgcacttagaatggtgcctagtacattgtaggtgctctgtaaatatttgttaattaattaatgtccaaggtcctgtgccagataagacatctcaaaaaactcatagtctaatgggaggaaacagacacacaacaagtataaaacagcgtggaagggcaatagtagagatatgtatagggaacatcacaagcatttagtttattctgaatggctcaataaacacttccagcaaatggtaaagcctttgctaatcccttaaggataagttggagtctgtcaggtaaaaagaggtaaggttttccgagtagagtgaatctttgtgcaaggatatgggatcagaatcagcatgccttgtagaggacactaagatgtgatactctgggcagtgagaactcatggaaggatgcttgttaagggagtgccatgggcagatgaacctttatggagggaatactcaggctacagtatcaataatggatttgaagaagatgaaattggaggcaggaaagataatggaaggttgttgcatagtacagggaggagttaaaatgcagataaaggactgactcataaaagtttaggaatttaaagcatcacgcttttttatttgatgggatttggttagtgagggaaggggaaaaaccaggtgactccaggagactgtcagtcagtgcctcttgcacattttttttcctgaagtcaattaataactgtctatattgatcaagtgttttgttgatttgttttgtttgatctattttaaaatccttacaaggtcccattttccatgaggaggttaggaggggaatgagaaagtaagagaacctatctctgcatcactagattgagcttcagatagaatttcaacatcatatagataagataaagcattacgtatatattatattacattgatatcatagaagagcaaaactgcacatgcataagaatttatacacctatattttcgcattgagaatagaggccccaaatgtatttcagtttgcttggcagaaaagagtagaaccacactccttgatgtgaaattgcatattctctttgtgtccctctgctcttcacataccttgactcacacatgaataggagaatagggcaaagcaccatctccctatagcgttcaaacggaatgttctagaaatgagtggagttggccggggcccactgccctctgtggttacgccactgtgggtgcagggtaggtttgggtgtatgccgaacacctactgcccatgtgaaggagagcaaagcaatctgttgatgaagcagtttgttgtcattgttcctattctgatcgttatttgaaacagttgtcttggctatcgatttttgcaatgatgtttttgatgtgtcatttgaatgtgaaacttaaaattaagaatattagagttggaagagatccagaatcatttagagcaaatctactgaatttattgatggggtaattaaaggccagagaggcaaaatgatttgctcaatgtttgtggcatttcaggcagaaccaacactagacgttaacttcttgggtcattgggtcacactttggttctctttgcactctgtgagttatgaggcacctagacaaggaaaggaaagccatttttaggaagatgtgtctgttattatagtaggttaggaaggaagattgaagtttagaagatactgtcgtgtttcagacctgagacagtgaaagaggacctgaatagggtggagtgaatgggaaaaagaaagaaaaggtgaatatgacatatattcaatatatgtcaagataaactcataggacttgtgatgagttaggtaatgcagagcatttgtacttggtagttagaatatatatgttctatataggcaaaaacacatacacacacacaaccctcatacatatacctatcacagatacactctgtattggatattcataacatgcatatcactaataaaaacaaaacaaaacagcagtcaaaacgttattttctcctttaatctttgtaaaaacactgagagatattgttgttccctttacatagaaataaggctcaaagggtttaaatgtttgcctaaagtcataaattagtgtgaggagctgaggtgggacttgaagacaagtttcttaattaaaatctgttctttccacaggcgccctttcactcaccccaagcatgtagggtctgaattttcttctgtggtgaatttttaaacctgttatatcagtctattcctttattttaggcagtgcatagtacggctcctcagaaaacatatatcccacaccctcttatctgcttttccagatttaagttgccttaagctattggtttcctctgtttgacagcatacaccatagtgtttagagatgtagtgcattatggcattaattttgcctggggtctttctcgagctattcatccttcagcagctggaggagagccatatacactctaaatctgtgtactacctagcgaaatctgtaaagccaagcttcccatccaggtcatgattcactgcatcctaggagtttgtgcatagaagcttgactcttcagtatggtgtctgagtaacagatcacattcttcatggatgtgacagttgtggcttctctttgggctttgttaggcataggctagactgttagacatcttacaaatctcctctccccacagtgccttacacagcaccttgtctgtattaattgctcaaaaacattgttgaatgcatggttaatgaaagttattattggcattattctgtggacttttttctttgacatttaacatttaaatatatacagatgcatatatatacacatacattatatatacacacaaatattaaactatacatatgtattctccatacatttcaaatatacataattgataagtcagtacacagataagttgtatgaaatctgaaacatgaagttaaggttttgtgcaacatgagtgctttatattttagacagattagctggggaaatggtagtccaggaactaagcacaactctataaataagggcaaccaaatagatatgagggatctagagataaacattttcagtttagaatactgtatatttcttttttttgacatatatttgaaaagagagaaaacggtaaacccaggagcagtaatttctaaagtaataaagatttacttacatggccttctgttccttttgttgataaggatgaatagaatgatataatgccctgttctctccctctagtttctgggccactgttatattgtctaataatatccactcataggacatagggtggcctttctctctttttataagggtataattatccagtagaaagtcaatattcctttcatcaatttttaaaattacttaatttcttttgtttcgataggtataggccctgcacggccttacaaatgagtaagggatacagcccaggagcaatgtccagctgcactgtttttacttacataaactcatgaacagtgtgtcagccagctttacacactgtgaccgtgagggatgtattttcccaaacattactgatcctttattgaaatggaaaatttagtttgcatgtggaataatttccctttgcaagtcaattgtgatttattaaatttccttgtccttcacaagatgtcaatggtgttatctcttgatacacctcaggacacgtcttgtctgtttgtcgtcactaggccttcatgatatacagcctttatgatatgtcttcctgaactctgaactcttgtaggaaatggattctatcatataaaactcagtatcctccaaatatgcctcagatgaagtagatttcagataaatatttgtaagttaatgagataattttatcctgtatgaaaataattctgaaatggaagacagcatgtagatacatcgtaaatattgttcactctatgtggacatctctccatggcaaaagaagactcagacaaaccaaactattatactaccaccaaaaaagtctgtgcaattaaaaaacagtgcaacacaatgcaacaatgcatccagccatgatggtctgaataggtgcaatctcattaaggcagaacacccccatgactgcttcatcttgcatctgtggtgcctagcatggcagccagtacatggtgggcattaaagatagacatttatgaatgaatgaataagacgccccacatttaaaactcagaaaatatgttttatgctattagttttgcttgaggtcccatttagtatgtgttatgtgtgaaggtgggaatagtggcattgtaaaatggaaggagtacatttttgaggtgattagccatagaggaaaagctgtaggaacctatttgggcaaatgacaccactgtgcaggaaatctctaaggagatctgtaattcttggctttagtacaacaccaaaaaaaaatcatccataccttattatgcagaaatttcccattattaagagatgttaatagtgaatatgcattcatattcactattaagagaatgaacacttattcagtatcccctctgggcagagctgattgataaacatcctggtgttaggcaaagactatagaaagaaggggtaccttgtgtaaaatgtaggttttctctcctgattatgatttttgccattactacaaacaatatgttgcatttgcatacaacttacaattttgaaagcatcgtctcaaacatacattattcttcactccatcttccttgttaggacaagagctgtttttctggtttcatacatgaaagcctaaataatgtctctcacctgggaaaaagtagagacaggactagagcccaagccttccaggcatccattctttctgcctgcccacttagacttaaagtaagactctgacagcaggctcttgaatttacctttgcttatattataacaacatccaaagcagggacaaccttgatgaccttgaccaactttaacacttgtgtaaatacaagaaaaatcaactttgctaaacttgtggtgtgcccataggtgacagtaagctaaaatgctaactccatggcataactgcatttgctacatgcagacagatagagttgatgtttgtcatggtatgagcccagggaatcagacaagtacttctctaaactcttcccactcaacttaatacaaggtaagcaggtcctgcagtagttcaaggacctgctgcacactgtatacagaatagggtactgtttaacagccttttatctgtcatacgtttcttttcacttactactgttactgtagcctgcctccacatcaagcttcgctgcagaaaggagatggatagacacctttaaagaattattcttcccccacttcttccctccatttggcttcctcttagactgtgcctcaagtgaagacaaggctagttacggcctcattatgcaagaggtcagagtgagatgtgaggtgtctacctttctctctaagcagatgcacccaaccaaagtgtgcagaaagaaccactgctgttcctactacacgctgattacagttgcacctggagccacattccagcatcagtgtcattcagatcgtcttagttctatccaagaattcttgtagaacctaagaaggagagctgatcagactaccagcacattaactcaaacatgccacggagtctaaataaatcaagtaatatcatacttgggataagatttaatatactgagtttgtgggcgagccacgatgttttgaatctattccatatgcctaaaatttggtgatgatatgtcttatttagcatgagttctcaagagtagccttagacattgtgcatttccgtgtcttgatatttggacattttcttgtgggtgtcctgaagctcttcctcactttgaggtgacattgaattgcaaagtctagacttttctgaaattgagaattatattatgggatgggattgaaaccatggttttctattgttttgcttaaagctaactttgaacaacaacagctcagctttagaaggtgttttgcctataaaaccattcacataatcaaaagctgaagtgtatgcaggatcagcttaatcaggacagagttatggatagccgtatacttccctagtgtttatagataggattgtgaatttggaagggaaacccagtcagcctaaattacttgtgttataactggagagttcactattctccattataatggggttttgaagatatgatatgcctacaaaactcttttttgtttttaaacttactatcttttttcattagtaaatatgtgaaagttttagtatcagtgctttttgctttgaaagggaatacaattattaattttacccatttcttattatgatattgcaaacagacaacagcagagacaaatacacatgtgtacccacttgctacctatataaggccttataatcattttatattcactatagatcgtaaaaataaacactacagactctgatgagggtctctatactcctctgagatatcgaccccattgctccttgagaaaaacactatcctgaatttagaagttatcattcccataagtgcattcacattatattttagtacatatttatgaattcagaaacaatatataatatgcttttgccagttttaaaaatccacataaatataaaattgtatgcataatgctgtgacatttaaaaatctaagattatattctttatatttattcctattaatacatgtagctctagctcatttattataaccgctgtatagtatttcattgtaattatatggcatagactatgctctcttttagatagacatttaagttatttctaattcttaactcttagaaaccatgttatggaataatttttctggagttgaaacagaaaagaggaatttggggacttagtgcaaggtacttgcttcaccatttattgtgtaatacaagttttaataacagtaaattaaagtattagaatgtgtgcattcaataataacattttgtaaaaatatttcaaagatttcaataaaaattaataaacacaattggtagaatatccctctgcctgctattggctttgctgcctccctagtctttagttccctcaacccagaatacccttggaattgccccttctgacttggctggccctgtaagggcttcctgtcttcctgctgagtgtatgcagctttgagtgaagttctttctgtatctctggcaacagttttgttttctctctctacccaccttgcccactctctgactgcttcccatcccaaaggagagatgtgttttcagatggcacttctcatctctactcagcctttttttggagggtgagtggctagatatgggcgatggggaacacaaagggaaatgctaagacacaaggggttttcatgagtggtaaaatgactggagggtttgcaggaggatcagggaggagaaagagtaaggaaaggggaaggagagagggaacagggaaaaatctaggaggggtggactaagatattatagtttcaaatatcagtatttatgcaaaatacacaaaaatatatgctagtactttaaaatggcacctattaatggaaaattaaaaaccggtgttacagttagtagtagtgtaaacacattttcaactttactagataatgtaatatttctttccaaattggctgtaccaatttacattcccaacagcagtgagagaattacccattttaccacactctagttaacgtttgacctttatttgattttgtaatgtttgccagcctgataggtttgatacaatgtttcattgttgtttaaagttataattcattcactgattattaggactgtagtgaatacttgtccagtgcatttttataacttttataatttttaaaataaattttattacaaaggtagtatagttttataatatcattaaaatcattaaaaacacaagcaagcaaccatcagccgccacataaacatacctcagtttgacatatgtatgtgccaatgaagccgttaccacagtcaagataattaacttattcatcaaccccaaaagtttgctcctgccccttaataatttattcctctctacttattgttacgggatctttggggtgtcaattttctagccagaaacctctgtgaccgtggcgcctttacccaggttcttgtcctgcatccaggaagaatgaggtacacagacaagtgaagagtgaacaagataaagatgagttttatttagtgtcacaacagctcagaggagacccacagtgggtggctcctctctgtaggtatgtcatccgtggagtgttcagttctcagcagagaggaggctctggagagagtagcctgtctctgcagctggttgtccagatggctgcagctctcagcagagagggtagctcctctttctgctggttgtcctgttgtctctttgcccccttcggccatcctctggctgtcctatgccctgctctggctgagcccagggcttttacagacctcagaggggaagagggaggaagtgcctgccaaatggtccatgggtggccattggtggcccagaagaggcaccaagactccccactctggtctgcaggactggcgtcccagcccccagccttcaggccctcactggcctgaaagtggggccttactgaggaccctcctccttctgctcaagaatcagtctgcctcctactgccattcatggccctggtctcggccccaacccctgctctgatcttggagcaggtgccaggagcagagagaggccaggcaacgggagcaaacaccccccccccagcctataaggatgggggtccttcctgggggcaccgtgggtgccagtggcagagacgacccagatcctgcacctgggagggcaggcacagctgcacccagaagctcccaccctgccaacttggaaggggcgggactcctacttgtccccagctcctgccttctccatggagtgggaggcccaggtctgcagctgtgggtcaggtggctgcagctgcacccaggagggcagattctgcctgttcttggcacccctcaagagcacaaggaggcttggatccacagccgcagtttgggcggctacagtggcatccagggagctcccgcccactcagaagggatggggctcccacccgctccatggagtgtgcagccccagcagtgcctccctgctacagccagtgtgatggcatcaactactgccatcactaccaccttcctccctccaatctcttggcaaccatggatcttccttgatagttgctttcagagccttctgtgttgtaagtgtgtatcaatgggtcattccttttattgctgagcagtattcctttgggtggtggtactacagtttatctatccgctcacctattaatggacatttgtgttatttccagctgtttaattgaccacttaatctttcacagaagaatatatatatgcgcctataaattttgtcatatgcaaaatgttaaataggaaataatccctgttagtattgtaatcagtgcctgggacaggacgtgcataggtattcagtaagtaggtgttaaaggaacttagacatgttttggcaaaatctgcaatctaattaggaaaacaaagcaagcacacctaagaaattaaagtgcacagagcagttgacctgaatggaaatcagtaatataataatatccatcttctacccaaaggtcatgacacccccccaacctcgccatcagcacttacttggaagtctcccttctttcagttcaaggcatcctaaattaaatcactcatctttcactgaaaacagtccctccactttcttcctgttttgttcaaatcttttcctaactactgagttcaaaacctgaattatttttgtaatgtccctctgttcacatttgattagtttccaagttctggttattctgcttcctaaatgcccccactttgttgccgctatgactgctttagtttagctctaactatctcttagctcagcacttagtcatctatcttgcctccttgactggctttcccctctctaattcagcctccacatggtcatcacacacttttctgaagtgtaattttgaccatgctgctttgttgcttctacctctccatttgctcctcattgcctgtaggctaaaattcagacttcgtgggatagcactgaagatgctctatgcttttcacgctttctccctgacctctatcatgcagaaatatttaactgcttgcaggaacgtgaacacacgatgccgtttcccatctccatgccattgaataaacctgtccatccatcttctggaatgtttcgccaactttgaccaggagaaaactcagaactcagatttgaagccttcttttatctcctgcatacatgtacatttacagaaaactcagtaaagttaaccactcctcatctatgccaagtgtctacaacactgcccttgcactgttgccagatcttccatgctagactgtgaccacagcaagtggctttttacctttgaatctagtaacaagtacaagagcagttacctaataggtactcggcaaatattttgggtgctgaataaatatgagtaactcaaattatggtgctcaataaatacgaataaatgaacatgtgactgaaataatctgatcatcaagttgtaatatcccagccaacatatgcctcttgtattttacttgttttcacttgtcaccaccaccttagtcaaaaaccatttaactttcttcctgaattgttgaatcagcttgctaacctatgtcagtaaattatccctcttttctaaactatagtaacacagattagctagactaatcttgcaaagactgctttaattatgttatttccccattagattccactgcctggctttcaaaagctctagctactctaataatcagacttttttctattgtataacacagtctccatttcagctaacccattcttcccacttgcccataacaagctctgttccttccagcttgtgtgccggtgctcacatttatccttttgcctgaatacccttgtcctctcttcccatctgtctgaattctactgactatttatgccttgctaaagttggcacctgcatcatatctcctttctatctatatctatatctatctatttatatcacatctccttggaacttctgaactatagcaatttgctattcaaagcgaatagaagttacaatgtacatattttctctctatatatcggaatctatattttcccccctaccctatactagaatataagctccacaaaggcagagaatttttgtctgtattgtctgtaaaggaaagccaagggctgagacacacaatatttcttaaataagtgaatgaaagtatgaaattaacagttgcatggtgctggttcaaataatcctcatttcctcggaagaaaagcactttgaagaaagtatgaacaaatttgaatttctgaattttaaagtatgtgatactttagaggcaatttcttgagtggaattacaaaaccatattcacttatctaaaatgtagcattaaagttctgaattttagaaccacttctgaacgtcatgatttctgtacaagatagtgtcaattagtcaatcatgttatctggaatttttatgcattagtataaattgaaattaactcaacatagttctattacagaagtggataaatggagcatgaaatgatatgagaacattctactcaaagtaaaaaagtagattcaggttcttggatccagattaatgtgatgatatctgtattttgacaggggtaaggccattttgcaagtctttcctaaattccatgattgttaaaaggaaaaaattaaagctcactctattgaagtccttagctcaaaatttgacacgtactagatatgtgttaaatagttgaatggaagccattggagaaccattttttttaagaggagggggacttcagaagggcagtgacatagttccagttgaatttgagaaaaaaattagggtaattgtctagagaatgatatagagagaagacagaagatagtaaaaccaggaagttctttgttcattcatcaaggttcagttataaagcacctactacttggtatatacctagggctgagtgaataactaagattctgttataagtatagcctgctggaaaaaatttagatgaacaattatgatgacaaaatttaagagtgattagagggaaaggagtgaacactttggaatctattctgctagaaatgctgattgtacttggtgaacggtgtaaaaattgagggtttgaagacacactccagtgttctgatttgagagaccaatttaataatatagttagagataagagtatgagaaagtatgaaaggaatgatgatggggttggttgtggacatatgaagttagaagagtctatagaaaatccctgtggagataaccagtatagaattataggtaagttctggtgctcagagagatatgagggctgaggagggatgaatttggaattaatcaacaaaaaatagttgttgaaaacataaatccatgtggaatttccccagaagggtgcttacctcattgagatgaaataagggctgaggctgcagataaggacagcagaatgttaagaggacaagaatgacactcaaagacagcacatcatggctagacgtgacgtcaagaaaccaggaaagagtagttgtatcacttaaaatggtttcaactacaagtaatagagaacctgacccacccacttttaaacaatactgtaatttatctgctctcataactggaattccagaggtagactggcctcaagagtggttgatccaggagctcaataacttcatcaagaaaccagtttcctttgtctccttttgcactgccatccacggctgtgaatttgtcctacatctgtttctcctctggggatagaatgactggtaatacaagtcagactacatgctccctcctttatattcagcggaagaggcagtttgaataggaacctctcctagaagaatgaagaggaacttacccccaagtctccagcaaagtgctccttctgtcccattggcacaaatttggtaaactaattattcccaaaacaattattaatttagattaagaattaggcctccatcttctggggctgagggtagatttagcttctttttttttttttttttttttttttttttttgtgaaatggagtctcactctgttgcccaggctggagggctgtggcatgatctcggctcactgcaacctctacctcccaggttcaagcgattctcctgccacagcctcccgagtagctgggattacaggtgtgcgccaccacacccgactaatttttgtatttttagtagagacggggtttcaccatgtcagtcaggctggtctctaactcctgacctcatgatccacctgcctcagcctcccaaagtgctgggattataggcatgagccactgcacctgtccagatttagcttcttttgaggcaccagcctttgctggtgagaggtgaatacctgaataaaaattaagattctgttaggtaaaaggaagtgagacagttaatgcatagcagacaacaaggagcactacagaagcattagtaagcttaggagggagaaaggttgaggagaagttattaacattgtcatataccaaatgggtcaagggaagaattaaggagaggcggaaaaatgcccatgagagcagtaattagaatgtgaccccagagacacaccatggattctaattaaagtggatagagggtgactgtgaggggaaaagtagttatgaggaattgagtctagtttcaagattctgagttttgaaagaaacgataaagataatagctagaggggggaaataatgagaatcaggggtttcttttttgttttgttttcatgatcatcaattaaatgtttcaaagttagtcagtgatttctaactgggctctgatgaaggggctttgagtgaccagttgtatgaacttagtcaacttgtcaagtctctttgacttctcatccattgaaagggaaaaaaagaaagctgtttttatcctatggagttggtggatttcagaaatatttttcttcagttttcttttattttatttataatttcaacttttagaatcacaagttacatgtgcaggtttgttatatgagtagattgtgtgatgctgaggtttagggtgtgaatgatcccatcacccagattgtgaacacagcacccaacagttagttttcaacccttgcccctccctccttcccacctcgattagtccctagtgtctattgttcccatctttatgtccatgagtactcaatgtttagctcccacttgtaagtgagaacatgcggtatttggttttctgttcctgcagtaaattcacttaggataatgacctccagctggtacatacataccatggaatactatgcagccaaaacataatggaatcatgtccttttcagaaatattttaaagaagagaatattgtttatattttaggtgtgtgtgtttaggagatattttaaatgttatactccatcaagtcttcaatacatttgggggaaacagaaaataaactaattttactttctacctcattgtttggtcttaggctaaaatccatgttttacggagaattcaagaaatttttaaacttcaggtagaactgtgttttttacaaatgtatagaaagcatagtgcctaatgcatggtagaaactcattaaatgattattaaattgttctacttttcatatttaaggatgaaaaatattagagattctccatcaatcagattataaaattctttatactggagaaatttcttataccatattttttatttgtcagagaattgattctaaaaaacaataaaactatgccacaaaaaaaactagtaatattgagtagtgcagcaaaacctgggtaactaagtctaactaggtaggttaattgaagttactggaaaatttattcatacattcaacaagtgttcattgagcacttattatgtgacaggccttgtgctaggcttcttgctctcatagagtatacattctagagggagagccaattaaataaattaatatacaatatgtcagggagataattgccttgaagaagaataaaatgagataatgggatagtaggcaattagtacaaatgttttcattttgtatgaggagagcaggaagggaatctctgaacaggggatgtttgagctgagacacaggacatgaagaatggctgggagaactgtgttcctgggaaaggggaaatcaagtgcagaggtcctcacctgggaacacattgggtgtgtttgacaaacagcaggttagactggggagatgggatgtagggaattggggctctgtgagtgaggggaatcacctaggagatgaggtcaagaaatggggcatcagtgtgccatataaggaagcaaggccttagagactgtgggaggaatttgggaactactctaagagacatgagaaggcattagagggtttagagcagaggactgattgcattgagctggttatcactctaactgccttggggagaataggctgttctagggcaagtgtggcttggggagataaagttcaaacaaatgtaactttttgtttccttgaaatatttaataaagtaaagctacctattcagggagtttgtgcaagtaatagcactgtcagatctactacaataaataggtgataatggtttgtaattagcaaagtaactattcatatagaaatcattcagttgatgctagttgactaatcactcctcatgcaatcttactcttgtatttgatgaactttgttttgaagccagtgcaatatgaagtgcaccatccctcctcagcagggacaaattaccagattattgagatcagtctccttaaaattaatgttattataacattctgttgtaaagaaagaattatatggcaggtatttttgtcaagcttttaggaactgttggagtgctttaaatttataacattgtgtgttccccttaaatgttcatggttggaacacatctaattttatgcaatatttaaaaacttttttggcagtgagattcatagggcagaagttacagaatgatttcattttggaaaataagtgaaaagtaaagataacacatgagcccaataaaatagtccattttcttgatttttttttttttttgccaatatctttttcatgagggcagaccatggtgcattattccacccccgcccccagcatttgcaaaccaccagtcatttccttagggaccttaacaaacggaagcattgaattctcttcagtcagtggtgcctcccacctgggaaaaagacagctctgtaattgaatcccagcatatgcattgtcatccctcctgcctgctctcctggtgctacagcaaattaaaagatctccaagggtcagcctgcctcccagctgccagccccagtagacatgagggagccagcaaggaccaggtggagcagttttgagttgcactaaacacacggcgcaaggagattggccataacttcccaattgaatatttatgccagccacatttctcatcttgctctttggcactgaacacagggctgagctcctgctgatttcagggcgctgcattgtgtttggatcagctttgtgaagtattacaccagatgagaaatgggcccaagttcctatcactaagacggctggttgactttttttttttttttcatggagactatcatgttatagtatcaaaagctggggcagtgagggagttccctgacatctcctgacactgctgttacatgaatcgtacacaacttcaaccattgcttgctatggagtggaactttaatggctcctattcagggacagacaaatatactctgatttgtatattactgcatgttcgtctttttttttcaaggttttatttttgtttgctaaggtcagggacttatcacagggccctctcatcacatttcacattctgatccctcgtggcaggtgctaacttccttgctctcaaactgaatgggttgagtaacctcataaagatacagaatgtcttggcagagaaaggaaaagactacagaatgcctgtctatggaagagtctcagtgttatttagttattagttagccctgtaaacccgagaagatatttggaaacccatctacacacacccattaactttttctaggtggtggatcacagacttaaattcatgttcctgattcctaatccatattgttatctaataaaagcacattccttatgtagattttatcatgaaatttgcttaaaaaaatcattctcttcaattgtatttttgtatagcataataaactatagatcagtgaaccactgaacttcttgtgaattgctggttctaaaatgaaattctcaaatgattaaatttaaataattaggaggcagtgataatgagaagccaatataagttctgtaggcaaaagacatgccagagtaacctcatagtcttttgtgacagcagtaccagaaaggaagagcaaagacatgcattacacttagaacacatagaattcagccaagcgcatgccagaggctcggatatttatgagaaaaagacagaaattagctcaataactattcccgatgaactgatataaatctggaggtatacctctagtaccctctttaagacctgtccttggcattgtgcttttcaacatgactacaggcatgcgccaccacgcccagctaatttttgtatttttagtagagagggggtttcaccatgttggccaggatggtctccatctcttgacctcatgatccacccaccttggcctcccaaaatgctgggattacaggcgtgagccactgtggctggccaacatgtttattgtgagggcattgagggcatgctaagaaagtttgtaggtgagataggaacttcactgggaaagaggacattgaacagcagaatcagaatctaacagacaggctgaaacaatggactcaagtccaacaagatggcatttaggagaaacacatgaaaagctgttcactcgggtcccaaatgccaactgcatgtgtagtgagcgactgggaggacacagggctctcaagcatcacatgggacaaagacttgagaatttcactggctgggtttaggcatgaattaggactgcacagtgtctgaggacaaagctaatgtactcttcagtctacactgacaagtgagagttgtaatagccttgccatattttggaccacaactgtgtgactatggggaccactgttaaagaagaatcctggagatcttatggaagagaatgctaggaaagtaaaagagtttgagatggactgagtggactgagggaattggagatgccctccctgagagagaaaaagctcttgggcaggacaggtcattgatgtgtgaaagactgttatatgaaagaaggtttagacttgacccatgacaataaatgaattttcttggaccaaagtttcagggaagccccggatagaaaactttctgatagtataagttatctgaaaatgaaagggctgcctcttaagaaaatgattttcttgacactacaagttttcaaatagacaaaccaggctgttttattgtctgtgttgtagaagtgattgtagtaataaaatttatgaaggatgcactgtaggccaacctttggatacttttttttgtgtgatcttatttaattcacacattagccttccagaggatattattatctccaatattgagactaaggtcccatgactggtaaatgaggcagagtttaaaattcaggttttagttgacagtagagactcttaacactgctctcatagccttaagcacaatattccattttgcttttgaggacatttaatgtcccttcagaactggaaatctgatcattttaataggttaccaccttttttttgtacattatatcaatgctttgctcttgttagtacaacattgggaaacctagatttgtttatcctgtgaacagtttgatctttactggtagagttttttcttagaaaaaaacagtgatatgtttcttagtacaattgatggcattaaatgttttgattgtggtcttatggctttactccactacttgctagaggaaaataattttctgcttcattaatgatggtttgaactgatctgacagaaagtgaatcctgtgccattcaaataatgcttggaaaatccattggatttttgtttatttctttgttttccgattaagggggacttttaaaaacaaaaatttagggaaatgcttctggttttcggaaacatttattcacatttcttggggttggagagaactgacatgttttaaatttgttagagtcatagtgaacaaaggggaagagttgcaggtctcaagtctagagctgtaggccctggaaaggaaaaattgcaaaagtgtccagagaacagtgtgttcatggctttatttgctgttagtccgaggcctgaggtcaccctggcagatgggtcacttgcactgcaagcactcatgctggcctcagggacagggatgaggtcactggcacatttcaagaacagcttcctttctttatgcatacaccactttcctggtccttgttaagggtctccttcctcttgcggttcccatctctgcagctgaccccagttctgactgtgtctggggaaatgggttggtgagggcctccttctgggactatctgtccacctgatcagtgatatagacactgtgccgcggagaggagccacatctcccagccagcctgccagcagagtaattggctcagggttacaagttgtgacaagtggtttttggatcctaatctgaaatatgtcggtttaacatgacctggcattgttacctgatgtttttaaatgcccgtactttttccagaaagtgggaactgatacacagcagtggcatgtcctactaattttcataagatctaaattgatcaggatgctttattactttttttattccacagtatatttttcctctttgactctaaagtgcatgtgcacgcgtgtacacacacacacacagacacaaacatgtgtgcacacacacacaccccccctcactcatttgggagatctaacactgcacatataaatattgaaacttccaggctgggcgcggtggctcacacctgtaatccaagcactttgggaggctgaggcagttggatcaggaggtcaggagttcgagaccagcctggccaacatggtgaaaccccgtctccactaaaaatacaaaaattagccaggcgtggtggcacgtgcctataatcccagctactcaggaagctgaggcaggagaattgcttgaactcggaaggcggaggttgcagtgagccaagatcgtgccgttgcactccagctctgggcgacagagcaagactccgtcttgaggaaaaaaaaaaaaaaagaaatttccacatgcatttccatcagagaactaattccatctccccttacctagccccttgctctcctgcctagcctgtgggaatctttgaagattctttgtattctaagaactgtcccaagaacaaagtagatgctcaagcaatgtttgctgaactagactacatttgattccatgtaaccaaatgactttatctacatttttttctattccttttattcattagcacccactaaatatactccactattctaggaatagggaaaaaaattcatgaacaaattagaaaacaatcctaccctgatggtgccttacagtcttaattacgtgaatagaagctattgttgttattcttacttttcaggtaaggagaattgaggcttttgtcacataggaagaaaaaattatgagcaagtcttgcccttaaaatcattctgatggtcaaatgcaaatttgctattttggtggactcttgagaatgtgggaaatagacccatgtccactttatgcaaagagaaaaagctggcatgagtgggagtgtttgaacatcaagaatgcttttgcatttcttgggttgtgaacaactgatggagtcaacttataaataagcaggatcatgactcattaccaggatctgtacagaataaataatggcttcagagttcgagtagactctaaactacacagttttaaggcttcccctttcttgggtttgaagaggatatttcaaattattagaaagaaattccatctatgctacagaaaaattgcaggaaaaaaaaatccccagaaactagttgcatgtctttacatttttctttcttttccttttttaagtttttgtgtttatgactaagttcattttttatattgtctggaaagatgctaatattcaaatggcctcatgacataaggaccactaacaaggggttgaaataaacatcctggaaagaaagaatgactacctttaaatattatgagcagtgtacatttggcagagcagatccttatattgactttagtaacctgaaaactattcaaaatgttttatttcagggatatgcattcctgattgtgatttcatggtgtgaataaaataaagtcacttaaattgatagaaacagtctgagctctggcatcagataaacatgagttcaagttctggtgctgctactttctatctgtgtaaccttgggactgctgtgtatcctctctgagcctcaatttcttcatctgtaaaatgtggaaaattatatattaatacgttattttaaagattaaaggtgataatatatatatgaaaaaccaaacacagaatgtgtcatactgtaatagtccattttcatactgctgtaaagaactgcccaagactgggtaatttatagaggaaagaggcttagttgacttacagttccgcatggctggggaggcttcaggaaacttataattatggcaaaaggtgaccgggaagcaaggcaccttcttcccaaggtagcagggaggagaatgaacacaggaggaactaccaaacacttatgacaccatcagatcttgtgagaactcactcactatcttgagaatagcatgggggagaccgcccctatgacttgattacctccaccttgtctctcccttgacatgtggggattatgaggattacagggattacaattcaagatgagatttgggtggggacacaaagcctaaaccatatcacatacaatatatacacagtaaaaactgatacccattaatcatttcctgttgcttaaacctaaggtaaaatttatagattttacattctgtataaattggaattaaaaagaaaatgaagtgtcacaattaaattaatcacaaaacatataaaaggaaatacaggaatatttttgctgtattcttagtagccatagtgtatctctgttctctgaagacttttagttgcaagaaacattgacctacctaaatgaattcaagcataagtaactaaattgtaaatatctagataattgcatttcggtttcctgtggactggaatgtagaatgccaaggctatttttattttgaggtttttgtttttcaggggtaacattttctgtttcttctctgctccgttctatggtgtgtgcttcatccttttattcctgaccggtgttccttctttactcatcttgtatgtcgtccagattggctatgagcccctacactactttgtggcttcagtgatatcattaagcagatacctcccccaggatcccttattcgaattcatcaaaacccagctaagaatttctgatgacattttctagaatctttaagtttacattaatttctatctgttccactttatggacgcttactgtactttaattttctattcacaggacgctgctttgtcatttagatggtaaactccttgagggtagggccatgttttcttcactcagcaatcctaacacctgtgacagtgctttacatgtagtaagcaaccattaatttgtttgatcatttaaattaaaagtcttggccgggtgcggtggctcacgcctgtaatcccagcactttgggaggccgaggtgggtggatcacaaggtcaggagatcgagatcatcctggctaacacagtgaaatcccgtctctactaaaaatacaaaaaattagccgggcgtggtggcagccacctgtagtcccagctactcgggaggctgaggcaggagaatggtgtgaacccaggagacagagcttgcagtgagccaagatggcaccactgcactactgtactccagcctgggtgacaaagcgagactccgtctcaaaaaaaaaaaaaaaaaaaaaaaaaaagtctttgggttgggcgagtctagtagaagataatgactttacgagcatttagcattgactgtaattaagtctgttatcgggatgtgctacagatactacttggtactgtttttgagctcatctttgttcttggatttcacatctctttttcttctgtttgttccatgattggcaaatcttctgattcctgactgccatttaaactcatttatcccagtttttcacacttaaattcaccttgtcacactcacatgcttgatttcacagctgtttctaagatggttatggcttacacaccatttcactcattcatttattactgaaatgtttcttgagaaactgcttttcttgagaaactactttttcaggcagctttgcaggcaataaggatatagcagtgattcactggaatgaaataatacaaattaaaatcagcaaaggaaaaaggcatatggggcagagttcaggagaaaccggagtttatttccagttgtcttctgtccagagttgtacaaatagcacttatttgtccctacaacaatgtgcgacagtacatgcagagcgttgccaatggtggatgctcatccaagccttagtgcccaggggtcactattattggaggtcagttatataaacatgtctgaccacccatgtggctgaccttcattactgtttttagccccttcataagtcagactgattctgcgtggctcaaagcccccaccaggaatcacattgttagcatcattgttagcaaagagtatctggtgtggcccaaggctccagctaaacacaggtactctaataagttaagatattccaagggcttaaaggttatctcccaggaattggtcaagggacagacctttcttttgaatgtgcagggtttggacgatacactttttactgcacaagggctaacctcacttattttctttcttctctcaaggaccaagccctgcgccatctaaacaattgtttccaacgttgtttcctcagttttagttttatagtggcaatattaggtcccagttactctatcatagacaggaattatagtcaagtaatttgaacaactctttataaaatgttatttcattcttattacttcctttctatgcttttgaatatattctataatgtgcctatataaaaatgtacataattaattagcacatataaatgacacctaaataaatatgtacacatatttgaggtgcttactttttttattcaattaacaatctagtagtttggaagctactgagtttgacagctgtttatttgcatctttaataggaagggcacttcaggagttaacaaagataaatgaaaaagaatcttgttatcacagacatcctgtggcagcttcttttttttcatttgttttctagaagaacgtgtacacaaatgacaataagttaagacagagtgtgaaaagtgctgcagaggaaggtaaaaataatataacaaaaatatgacagtatgaccaggtcacttctggttttgaggaccagaaaaagctgtttggagaaagtagcatttctgaggtatttaaacattgattaagatggttttcaggagctatcagacaagaaagatgctgtacaagctaaaattgtgaaggggaaattgtgacaatagtacaataatcatttctgatacaagaaactgtatactaggtgattacttgaagataaagctgaatggagagttgtgaatgtctgggtaaggcattgggacaggatagtactgattatatttgtaaagagaagacatataataagagctaagtttggatttctcagaaatcacatataactttatttttgttcccctctatgcatttcttatgattggctttaataaagcattctgttattttcagaatttgactgatgtgtttagctgtggtttttctttttcctatttgatgtttgtggtgctgtaaggctctggggttgatgtttttcatcagatttgggaaaattttgaccattacttattccaatattattctgtatcattctctttctcctgtttttttgataatattacacaggttacttatactcaattaagtttccactcccacttttttcctctgtgcttcagtttggataatttgcattggcccagcttcagatatcctggtcctttcttttgcaatgtccaatatgctgttaagtccactcaaataatacttatgtaatatattgttattttcattctagaaattttatttagttcttttttaagagtttccatttcttctccctatgcaatatagagatacatactgttataaataaagcttcaatgctgcaaaagaaatggcactcgaatataaaattttctttttaattctcagcaaggcaacatacttctatagaagggtgcgcacttacagatagagcaatggtgagtgcacacctggacaacagagggaaaggggttcttacccctgaggcacatagcccctgctgccgtgtctttcccctattggctatggtgagatggctaaactaattctgattggctaatttaaagagagtgacagggtgagtggtctggtggaaaaaaatggttatccagggtggagaatgagtcagggcggagcaggtagtaggtaattggaatgagttgggatgaagcaggtaatcagaatgagccagggtgaagcaggtaattggaatgagtcagggtggagcaggtaatcagaatgagtcagggtggaacaggtaattgaaaagggttgctttatgaggaagttaagtttaaaagtagaaggcaaagaatcaatactgacatattgattctttgaaaagaaatttagaactcatatctaacaatacatatatttttatatatatgttatatacatatatgtattgatccatatatgacagatgtatatatggcagatatgtatatattaatacctatatatggcatataaatatttatataccttttgtgtgtatcttttcctataagttacttaatatattcacattagttttaaaatccttatctgctagtttcaacacttgggtcataaacaggtctgcttttatttactgtgttttctgtccactaggagtcatatttttctgctttttgttaggtcacaaaatatttataatgttctgaacattatatacaataaatcaatagcgattgaagcatagtatttcttttgtgttttatttccccaaaaagtggtagttctttcctctggcagttaaggtgaggatctgttcactgagacccttcctagagttgagcttaactggagctagactatgattagcccaacttccatccctctgaaaaatcactgcttttatatttcaacatttgaactggagaggcattgactgaagttataattttttagggttttttttagttaatatttttatttagtactggcaaggtccttatacatgtaactttggtaaattcactcattagttctagtagtttgggggtttggggtatgaatttcccagaattttctctataaacaattaaatcatttgtgaattaaagttttttctttctaaactttgcttattttatttatctttgtcttgtactggctaagattgctagtagaatattgtggtgaagtaggaaaagcagatgtctttggcttgctaccgaaataattcattatttcatcaccgaatatgatataagctatagacttttcaagagtgcttttatcagacagaggaaaaatacttcttttcttagtttgccaagaatttgtatcatgaatggatgatgaatattgtcaaattttttttgcactcatggaaataatattatgttaatatattgaattacattgatttccacatgtgaaatcagctttacattactggcataaagcgcacttggtcatgatttttttctattgctgggtttgatttgagaatattttgttaaatattttctcatctatgtttataagaaatatatatccatattttcttttcttaaaattgttcctttctggttttggggtcagggtaattctggtctcctaccatggaatgaaaagtgtttccttctattttttctgaaagaatttattaaaaaaattgttactaaaaaaaatttttttttaaataacagtagcaaaaaaagtttgctagtgtttattaattgaaccatatggccctggaggttttttgataaatatttaattataaattaagttttttaatcaatgcaggactatttagattttctatgttaattggatcaattttaataaatgtgtctttcaggaaattcactgattttgtgtgtctttgaattaattggcataatatagtttataatacttccttattaccctttcaaaatcattggtatctctcatgatcatcttttggatttgtaaattttctctattgttcacagattttgtttcaattatttctactctttattgtttccttctacttaccttttgtttaatttccttttcttcctctagtttcttaaagtggaaacttaggtcacagattttaaatccttcttcttttctaatataaccatttaaaggtataaacttctcattaatcactactttatttacataccacagtttgatttgatcttttcattatcattttgttctaaatatttattatatttacttgtgattttttttcctttggttcacaaattatttggaagtgtagtgtttaattttcaaatgttggggattttttatatgtcttattaccattgctttctaaattaattcattgtggtcagatactataccctgtgaaatttcaactttaaaatctatcaatatttactttatagcccacctatatcccaggtctgtcctcttgatagatgttccatgtgcacttgaaaatagtgtgcattttataattattgggtattagtgtcctggaactgtcaattaggtcacattggtgataacgctttccaaatcttacgtgtatattttttaaaatttatttttgtctacttttcttctgttggttactgtgaaaactgtgtcataaaagctccaactatgatcatagacttatctgtttcttcctttaattctgtcagtttttgcttcatgtattctgtatctctgcttgtaggcacatgttatgtcttcttgaataatttattgtttttcatcattttccaattcctttctttatatttgctgatagtcttcatcttgaggcctattttgtgtgatgtaatattagcttgaccatcttttttatgttaattatctgcgtggtatatcattttccattctttgaccctccacctatttgggccttatcacttttcaggttcccaggatgcagatgctgagatatttcctgcaggagtttatttgggttgtgctgtcagtattagttccttttgaggggcatgaaggaagcagggctggattcagagacgttgatctgtagcacagttgcagcaaaggtctcagctagtcttatgagaggccctagactatggtgacccttagggtaagagggaatagtcattttacccctccattgacctgtcagtagatgagggctgccctcagaaagagtgtgctcttgggcacagtgaccccgtttagttgagggcaagttttggaaaggaactcagtagaaagctaccagttggtaacactctcagcagctatgggaatgagttcttcaggatgtctgggcagcgtactaaagtagcacagtatgctgatatttaaagtgcttctctcatagatcccccgtaattaggtcttggttgtataatctaacctgaccatagtcagctttttattggagtgtttagtccattgatatttagtgtaatcgtcaatgtgtttgagttcgagtgtgccatcttgctttgtattttccatttttcacatatattctttgtttgcttccctttttttggcttcttggggttaattgaattctttaaaatgctgctttaatcgttctgttgggtttttgttatacctgatggtatttttcttaacatttaatgctagagattactccatgtaccctaaacttaacctcttatccaagtttaatataacacttcatgccaaatgaaagaaccttgaaaaatataatttcaccccactttttgctgttgttgtcacatattatataagtccctgtatacagtgcaattatttttgcttcaataggctgtctttcatgaattaagagaaaaataaaagataatcttttatatttacctatatatttacatttatgttattcttaattctttcctgtaggtctgagttttcctctggtatcttttctttgagctgaaggtcttcctttagtatttgttatagttcaggttgttggtgagaaattctctcaacttttattgatctttatgtttctttcttccatgaaagcatgtgtagcttctttgggctactgcacagttctttttcctagtttcctttcctttttcttttttttttttttcttttgaaacagagtctcgctctgtcgctccggctggagtgcagtggcgcaatctccactcactgcaagctccgcatcccaggttcacaccactctcctgcctcagcctccggagtagctgggactacaggcgcccgccaccaagcccggctgattttttttgtatttgtagtagagacggggtttcaccgtgttagccaggatggtctcgatctcctgacctcgtgatccactcgcctcggcctcccgaagtgctgggattacaggcgtgagccaccgcgcccggcctctagtttccttttcaaaagatggtcagcccttcaagaatcccaattttattttatcccagttgtagcacaatctctggagcaaacttaagtcaaagtcctattcctgaatgcatactaaaagtgttacaaaaccagctgtcaggacttgggctcattttttgactccctgggtcaacaaggtcagctggaagcttctgcttgatttggagttcacactttatttctagctactgatttcttccatattggtggcaggggatgccaaatttaggtaattaaacattttcagctctctagtgtgtagctaaggtgatcagtttaggagggtgggtatgcatatcagcttaaattgccaagatgacagaatctgcattttttcttacagtattatatggaatagtcataggattattttttgttatttttatatgtcagtgataacatgtttatttttgtgttagtaataacatgttttccatattatgttataatatgttataataacgtgttatgttattacaaaccggttatgaggcttaacttactagggaagcttttcttatttatttatgcatttgtttgtatgtttgtttctcatgaactggcatgttcatctggttgactaagtcttagatactggttttaattattactgtgagatcacgtggtcaatagaataatatttgtaatctattaaaaaataactgctgcaatttcaacttttctctgctgcttactgctctaccaggtctatggaaatgcaggacctagcaagtcctcatactcttgttggaggtggtgatactccaggtagctccaaactggaaaaatctaatctcagcagcacatcagttactacaaatgggacaggaggtaagtgtactaccctgaagatacccagaatcatatttcaatgtttgctttaattttacttcttagagatctgctaataaataaatatcttattatcaataacacaaatcagcattatatccaaggtatatataattaatattgattgttttgatgattttcttgaaatttttgctcctcctgtatagctgggaacattcacagagaatagatagcattgagcaacatttcaagggccttgaacaaggctttctttttgtatttgcccttgtaaatattccacaaactttagaaattgaggtcaaaggatttccaccctggtattagcaaaaatgtctgtatgtgtgtgtgtacacacttactactttttggttttattttgaattataaacatttttcttaaaaatgtttcttcttaaaagtgcttttttaaaaaaacacatggttttggaaatacaaaaatactcatatctgctgcattttcttttcaaagagaataacacttaacatttatttaaaaaggaattgcatttttttcagataatttctcattctattagcccataattacgccacagcatgacaacaataatagctattacttcagatattctgctttattatttttataaaatgtattcaacttgtcagaaaatatggtccaatttttatatcagaatgtgtgtttgtagagattatgtatacacttatatatagactgtattttaaattattcacaatttgaaattttgtagatccatctagccaataaatttagaattatatgacatttaatttagatattagcatttgccacatcatattttcttttaaattttaaataaatatctgtgaaattgatacacctcttcccatttattttagaataacgatgcaatctaaagttttctctgtaaggttgtgtgcattgcaacttacctgtatttaataatgttttacaccagttcccttgtagattaacattttccttgtttatgatgtttatttgtctctaaacatgcatctgtaatttcagctaattaattatgcaattgcaaatgtatagaatatttagtgatgaaaactagagtaaggtacatttaaaccttaaattgggtacaaataataaaatttaaaaactcaagcactctaggaacagactggtgaagttcactaatcatcatatgtatatatttgactgaactaaaacagacctgataaactaaaatgttgaagtcatacagtgcagttataattttggctaaaaggtcagaaaccagtgcaagcattgaagattattcatatccactcagcaggcattcagacaatgaacttttatactgttcctgttctcaggggaaaacatgactgttttaaacacagcagactggttgctgagttgcaacaccccctcttctgcaacaagtatgagagatgctggtacactgcatgtgtttgggtgtgtggatttgcccctctggattgctgtctgttgctatgtctatgtttgcatctcttttctgtgttcagtacttaaaaaggaaaaatgaacatgccttttagttgtttattgacagcagttggttatgatttcttatcaatctgtattcttagatttttttaggtcagtagtattagtattcttttcttaaatttttcataatttcacctaaatgatgcatatattggttttctaactatttatcagtttattacatgattaattttgcccaacaactgccatgtcaaacagcaattgaagcagaatggccacgcatgcaaacttaatcaccttcataacccacagactttcataatttaaattaaattcttaattaaggaaagatatttgctactctacagactttgagaaatattccaaagtatataaggtaacattaataatcaatatcccctcaccttaaaaattatcagtaatgaaagctatacatttaaaactagaaattaatattggcttttaaatttttttgaactatggatattaactgcattcatacctacacacatataaacaaactttctcctaaatcaaattctaatatcctaaaaaaaatcaattagaacaaagccatcacatttttgtggcaccacccagttgtagaatcaaaaaggagctcatgcctattggaaatgtgcatcacaaacaagcagttagaataggcacagtttgcctggaccttgctctatctctgatgaaattagaatatatgatcagtgaagaaaaactacttcaatatgctcattatttgtatttgtatagttttagttttaatgtttttcaattttatacttaaatatacttttcatacttaaaattatgttgctagaatttagatactcttaattctatagcagggtctttttctctaaagggccagatagtaagtatttttggctttgtaggccacatgtctgtgtttcagctgctcagctctgccatcatagtgcaaaagcaacatagaaaatatgtaaatgaataagcatggctatggtccaataaaacttcatgggccctaaaatttaaatttcatataattttcatgtgtcatgaaatattgtaattttgacaactttttaaacatttaatatgtaaaaaaatgttcttagcttactggctgtggtctgttctcaaatagtgtttaacacaagcaacattataatagtgaatatgtgcaataaatagtaatttacctctgatctcaaccagaaacatttttctaagtggtaagcatcaggctttcctggttcttaatgtcagtgttaaaactcgtgagtccatgggagaagaacaaggagttgtgcgtcagggccactgtggatctggatcttagcactacacatactggctgtgtcaccttgaacaagttacttaagctctctgagcttcaattcctttctataaaaggatgataataatacttcttggcaagacttttgtcttagataatgtatataaattgcttaacaataaatatcagtatgtcttcctttctttaattagtaatgcaataataggcaacagctttttaaggaaaatgaaaatatgcattgtgttaacagactccatttggtcagataaatttagttgcatttagaaatgtgtaaactgggctaaagaaagcaaacttttggtaacaaaatgttagataaactttgattattaataatgtagtcctggtagcatattatatgaaacttttaatgaatttcaatgacttttttttttttgaaacggagtcttgctctgtcacccaggctggagtgcagtggcgcgatctccgctcactgcaagctccgcctcccgggttcatgccattctcctgcctcagcctcccgagtagctgggactacaggcacccgccacgacacccagctaattttttgtatttttagtagagatggggtttcaccgtgttagccagggcggtctcgatctcctgacttcgtgatcggcccgcctcagcctcccgaagtgctgggattacaggcctgaggcaccgtgcccggccaaatttcaatgactttttatggtgggtgttggtgacacagactaagcatttgttgcatgatctcttctgttaagaataaactattttattttcaattcttggtttttattgttggaattgtatcacttcataaaattataacaaaacaaaaggcaaaagaaaagaactaatttaatagcttttcaagatcattccaataactcgagatgttcaaaaagattctttatttatttatttaagatggagtctcactctgtctaccaggctggagtgcagtggtgccatctcggctcaccgcaacttccacctcccaggttcaagtgattctcctgcctcagtctcctgagtagctgggactacagacacacgccaccacgcccatcaggctaatttttgtactttacttagtagaggctgggtttcaccacgttggccaggctggtcctgaactcctcacctcaggtgatctgactacctaggcctcccaaagtgtcgggattacaggcgtgggccactacacccagcctaattttttttaattgatataagtagatagtatcacctttagattttatcctactacattggtgctctttaagtatgttgcaatattcttgaaagtaatttaatgtgtttcactaccttaatcctaattatataattacatctgcagatttatatggaacatagattttcctttgaaattgttagcacttgtaaaattattaaagtgtaaaatatagagatagcataaatgcaagcataccttagcaaataaagcagaggtcaggccttgtgggctgtctgctctctgttgtcgctatttagctctgctattgttgcacaaaagcagccatatacaatacctacacaaattaacgtgattgtatttaaataatttgacttatgaacactgaaatttgaatttcattaaaatgttcatgtcacaaaatattctccttttgatatttttcagccacttaaaaaggtaaaaaccattcttagcttaaaaccatacagaaacaggccatgggtcagatttggcccaactcctgatatagatctatatgtgtatacataagttatgttgtacatataagaatgcatgccaattatatgaaaatttacttccagatataggtaattccagattctaatttgctgaatggtgtcaggattcatgatgaactacacttcatatacaaaaatacttagatctattgggagggctagggcagtatctaaaagagtaaattttcactttctcacttaattgaaatgtatcaagttcctattatatgccaagttctatgccaggaaatgggaacccagtgacctaaagaacacaataccagccatccaggaaccaaaagtctagcagagggaggcagacatgaaaataatgaatgccataatatgttcaaggtggatgaacagtggagggagggaatatttgattctacatggagattaagaaacaacttcaaagaaaaggtttgggggaatagaaatcagacattctgatcagctttagtgtttttttctatatttgaaatcgatatagaaaagaatcttctagaatttttactgtagagatgattgactcacatataatgaattagtatttgtgcaatagagctacctactgctgtgggtcagctcttttgtagatttttttttagaataaaagtcataaatggcttcatcaaattattttacaaaattaaatactttatgggtagttaggaatgaaagacacttggtaatcataggtttttgaatgaatatattgatccaaacaagcataaatcataggctatactgattattaaaaccagtaggcaatacttttactgcttccctgcagtgttataaattgtaactttcctgatctaactctttctgaaattctttgtagaaactaagctgtcaaaagcctctatatgtgatttttgtggttcagaaagaagtgtatgtggaattacatatatgtagttttttaaatggacaccttaaactggtgcacacacaccttaatctatgtttaaactacactgatttatgaagatgaaatgtaaatgtaaattatgtgggttacctttcgaggatgaagcctgaattgcaacatatgaatatgaactttgagttatgataataaaacagactttctctaaaagtaattaatcgacattaatgttatacttaacttctaaagacaatttaaggggacaagagtaagactgctttaaaaataagtgtatggcagtagggatgaaggttgggatgtgaagacaggaaatctgtgatttagtagccattactggtacctgacaataattatgtatgaccttgatttagtctatagataagaattatttttaaagctaaaaaaatggaaaagccaatcatgtatgtttatttatttttaccatttgactctatcagaaacatccaaattgcaagtagctaactagtggggagggggacctgacagtacaaggatttcttaacggttctcgacaaagtggtatagaaagtaattttatgccacttttagcaaaacgagctctgactcacttactttgaagatacagaataataatcccacaaaactcttcaacctaggacattaaaatattgtttcttttgaaatcaaaacatttggtgtgtaagcttgcatttggttgagaaatacggtgcttgtcttctataaaaggattttgcttccattttattgcatgtaatgattttggtttgctgtcctgaaaattaaaagccccatttaaacttcatgttaatttaagaaagacaaaatatagcttaacatacatttgaacagtcaaggaataggcactgataattatttggaataaggatttttacaagttagtagtaataagactagatttgtcttactaatgaacatttttccaatagtgtcttgcatatatttaatgaaagtttcctgccactagaaaagtatatagtaggtgtaatttaaagtgcagaaacaaaagcaagataaaagaaaaaagaatacatttcaaatatatttctagcttcagtgaaaataacgttgttttccaagctttatctataattattatataatgtttactttatactagttaactgttatataaatatcaagataatacttcttcgtattctaacatgtcttaataaatcagctaataatataaggagtgtctattcacttctctatttgagctttctactgctgaaaaaatagttgtttattattacctaatattactgctttttatttagaaaaatcaaaccaaaataactttgggacttacttagagatcaagaggtgcttgttgatgctcacttagtatgaatgcagcttattttctgtgacagaataaatgttttatttctacttttccattttgttctacccttgcataggaaaaacaattttacattaacatgtaccacttcccctcaaatgttagcatccattactttaaaattcaggcattttagtaaagctaacacatcttgcttgttactcctctcttcatatactttcccagggatggcttttctcagtaaaatgaacttcctttgtctatctttgactaaaagcaaacctttaaaattttaatgagagatgtttccagtataatgaattagaccaaaaactcactctctccttcttctttctccttttgacctccccaaactttcaaattgaagagattataatcaaaatcaatactaagcttttgaaattgggtctcaaagttgaaattcaaagtaacaaaactaatcttcatgtctggcttctagaccagtttcgttagacacagctcacattctgccacctccattcgcaagtaaaatctatattacaattagtataataaagctacttttgcaaatattgttcgtagatatagatatgcataaataggaatttagctgcaagtagaagtcagaagctctgataaactgtttcttagttttgctgaaggctttgggccatgactttttgtcataaatgttagaactcaattgtaaacattgcgtgaaatggagtagccttgatacccatctagattatactctgcattatcttcatacagacaaaaggaattcttgattagctagtgctgcctgagaggcagtatggtttaagagtttggattctgaacacacactacttggtctcaaattctagctcttacaagctttgtgactggggacgttatttaacctttttgtgcctcagtttccttatctgaaaaataaagatgatgatattatctactgactgttgagaggagtaaatcagtaggtgtaaaggacaagagaagtgcctggaatgtatcaagttcattttaagggtgtgctgttactgttacctaatttcaaatttgctacttcttgatgtcttttaaaatatcccatgaggaagaaatgggaaacatgccttagtatcctattccagtattttgcagtttttattttcagtgaagtatttttaattattctatctaggagataattgcattctcatcatgctttgggcctttgagaagagacactgtcatgagcataagctattttcgtacctgaaggcagcctgatacggtggaaagatcctgcatactagagctaaaaacctgagttcaaatcttagctctaccgtctcctgcactggaacttatgcaggcttttcagcccccctgaacttcagtttccttgtctataaaatggttatagtgatttgtattaattagagttgttttaaggattagctaaatgtacatcatctagcacgtagcaagctctcaataagtattagttttatagtagtaaatataaaggtgataaaatgattttataatgataggtttagttattgataaggtataaccataatagggattactgtgcaaattactactgaaaagtaataaccaccaattcattatttatcattatataaataattagactactcttcagatcattgttgctcttgaaagataagtcaaacttgagtttgcttttttgctaagaaaagtaatccaagtttctttactttctgtttccaacatttacatttctcctctgaatcttccagctgatcatgtcattaataaataatggatactaaattaacccagagctcactgaaggtctggttaattctgttcatgtggaaaagagcataatcattaccgcatggttttctatcaattaatttcttttctaacagaataacattactgacttgtgtgattggtgggtaccctatgactgactgttcttttttgtcctttggctagtcttcagctttcttctttataattttctgcttgcttctaagttatttaactgtatcattcatttcacttatgaccaggaaacacaagaggtgttaagtagaaaatagcttagtttatttccttgctgctatgatatatgtagtaaagccttgccaacaaagggaaaatatggtgtgaccaaatgggtaaaactttattctcacgccccatcacccttgttgcatctaataatgataataatgtgtactaggccactttgttaccaagcaaagattttgccaaaatacaccattaccacgtagaaataacatattaaaatgagttttcaaaaggtgtacttgctgctgtagaagtcatctctttttctacgcttatctctaaacatattaaaaattgttatgccattaaagtatgaagcccaacaccttatagctcgtgagtcttttttagagcttcattgagtgcttcatttttcattcattgtgtgaatgagcccatggtttctatacttttatatgtctccattgtattattatgcaaacttaaaaaatatcttcctgggaaaaataaaatataagctagtaaatttctttggtataattttaattaaatgtgctgaagactgatgatgattccttctttcctattgactatatgagagtgtgtttaggtgaattaaaaatacaaatatgtgtactgctgtttcactttttaaggctgactgcctttaaaagctggatttgagagaactagcttggccagaggattttagattaaatcctctaaatgcatgcccgtttagaagctaaattaacaaactctcttcatcaacttctcaggttttctcttgcatgcccatggtagctagaaatatcactagtagaacggacagagtctttgacataaatttagaagtaaaactcacatgtacttattcttctacgtagtgtctcttcttgcagtcaaaacagagcccttgaacagcagtgaaaccacagccacgactggagatggagcgcttgacacttttactgggtcaggtaaagcctttagctcgtgtgtccttacgtacacatgggggtgcatccacatcctcctcctcgggaataagcaacataagcatccaagctcttagaactttgatccttataaagttagaaacaaattcttattttttgagaactcatagtcatagcagtatgcattgttataaattatatttagaataatttcgcttgatattttagttgacagcaaaagtcccatgactttcttattatggataagtaaacttaacttccttcaaaattattttatacgatgtttaattaatttaataattaagttgtagtagctgaccttgcttcttcaacttacccatttgaattacataaaaactgccttctgtttaacattcgagatgtaaaactatccatttgattccttctaaataaccttgtttcctctctgggtttaaaaaactcctcctgtgtaagcagctgagtgacaagtattaaaggttaatgggctgcaggagagcacatgctggttgcattagaacccagcctctgagagcgctcgaatgcttcaaagagaacaatagcatgtctttaaacatcaatccctttaaaatataaatacctgataactcccagctgggcacaaagggcatgaattaagtagacttaattaaatttttttaagttacaatatttcttcggctttttatctggtgaaaggatttttctaacctgaggaggatcgggtacatcagaaagaagaacattaaacctaggaaagtatttgtggtaattgaatctcggttctgttattctactttaatacattttacatttatccatgtgctcatgttataaggatttttttgtcagtatgttttgtgtaaaaatagttgagatatgtcttagagtattatgtcattatagcatatatttgatttttacagatgatctaatttcactagttcacaccgattagaagcagttgccatatatttacataaatattagtactccaagaaagaaataatagtacataaactttgcacagagactcatatgtactaatccacatcttttctagcagtttacctgcctctcaggagatagttaccagttcagtattctgtattgcacacttaccatagcttttgtaaactttctgcaaaataaatctatatgaacaaattgttaaggaaaggaatggagttattaacttattgctgacctacttcagtagtatactacaagttaattgaaagctgaatgacaaaactataaagtgttagctccagaaggaatttcagagctctctgatccgttttgtagacaagagaagttaaaagtggttaaggccatacagtcaaggcctcacagctggtcagtggtataaacaaatctaaaaccatgtccttatgatcagtccctgggtcctttctgtaatgccccatcagaaaatagatggaaaaagaaataggcatgaacaatcatcctgtctgaaattcactcatattgtctttgtagatattttcgcaaatttttaaagaagtatctagggacgtgctgtccagtaaggtagccaccagtcatatgtggttattaaaatttgataaaattaaaaattttgttttttattttcattttcacagcaatattgcagtaagtatatttttattaattgattttgaaaatcatcaagtacgactagaggcgttaagttctcaatattttcagcaaccaaaatttaactgaattttcaagtttaaacacatttcaagtgtttaacagctgcatgtggttagcagctaccatattggacagctcagatacagaacatttccaacactgtggaaagttttattccacagcagtgatccagggcagggattatcaacatcagcactgttaaagacttgggctaattctttagtgtgggaaagctatcctctgcactgtaagatgtttggctgcatctatcccagaatgccagcagcactcccccagatgcttctttactaaagaagcaaccaaagatgtcttactaaagacaaccaaaaatgtatccagactgtttaagtgtcccctggcaggcaaaattctcacccccacccacttctcatcccattgagagtcaccaatctaagatggcacgataatgagaatgaaaccttaaaattgctttgacaccatcagagaaactgacaatagatattaaacatgtatttaaattaattttcagggcattttgaaaacaagcagttttgcttactgttgtatgcagaaatgatggaatgtaatgagtgttttgggtaatgtttatgattaatcagatgacagtggggaataaaacgaaattctttcaagtttgtacaagatattccagaaaccattctggaaatgaggctgccctttgtgggagcagtctttactttgatctgcaaaatttccactcactttaatccaattttgttgttcttttcagttatctgcaaatttctgaattagaacaaaatgtatatattttagtaaagaagcataaactttcctacgtgaacatcgtagttttctgagccaaaggttttctgttctatttagaaaatgaacatatagacgttaatagaaatttgtaaaggttagttgaggctttatagtgttgtgagtacattattctaatgatgtttagatttatcattagaagaaacatcctatatattcagaattgagttcatgtatatgagttttttctgaaatgtgtaattttggtctatgtcttatttataaaaggttactataatgaatatttatcttcatgattcagcgtctctttggaaaagccttctatttcacaatattgataataactgatgagtatagcccaagtcttgagtgtaccaaggagaaattctgtaaagaagagacgaaagaatatttaatcaagcgtcatttaactatatttctgttagagtatttggtattgttaatcgataaattagaatgaatatctttggaataacactactattatgaagtgcacatttaatgtccattgtgtagtagaacaaagagtctgttttcattaagttgatttgaaaatttaaaacgttctttaagttttgataaatatgtagaaaagtaatagtttattggtcaatttattttcaacgatacgtatcaaattcagttaaattctggttgctgaaaatatttagaagtaacacctttagtcaaatttgatgattttcaaaattgactgataatattcttaaaaatatacttacttcattatcgctatgaaaatgaaaatattctttccatattcagagcttataggcaaaatagcattttaaacacattaaatgatgcatgtttgtgaagcagatcacaagggttcttgtgatatatatagaaatatatcatgtttcataaaatagttagcaacctaaacctatttgtcacttttacaattctaaaagtttcgctccagaattattatgaacaactcagaaaggacatagtactttgaagtataactatgattgattagactaaactaattccagcactgcattgtgtactactttaatctttttgaaaagtgaatgaacagcagatggcctaataaagacactctgtgtgctcgctcagtgtggattggctctaagggaactggctcacactttagagtccatatatttgcaaaacatctgtcttagtcaaggcagatagttaaaggaaagagaaacttccaagctattggatatattccagtttggggctccatgtttggatgaactgttaataggacctcactttcacctttaagttcttctcagaaaagtattatctctaatagttcaaagtatatttgtaaatgtaattgctactgcattttgtgattatgttttaaagaaatcacctgcactttctttaattattactggaatattttagactgcattttttaattgttcatctgatgggtaaaatgagaggaaatagaataaaatagtattttaccattagaaaatgaagtttggaacatcacagtcttattttgtattgtatttgaattagaacagatatggaggaagagcaatatgtatatattgccatcatatttggtattttaaaatgtaaagtttgcaaattttaaaaatttacttttatattttgaaagcttaattaccctataatcaaagatatttattcatagcatattaatatataagagcatgctaatgatttgagaaactggaagaatgacagagggaatgatttgtgagacaaatgtttatttcatgctttttgcacaaaactcaaagcagtttaattaggagaaagtgtttaggcagctgtttccttttagaaaatgaatttctggaaacatgtatttaacatggtaatttcttcaaattggttatgtacactctagtgaaatgtatttatgaagcaacctttggtgcactggtatttttgtgtcttacagtaattacaagtagtggctacagccccagatcagcacatcagtattccccacagctgtatccttccaagtaagtggtcagtagattcttgctttaaattggcaaacatttatgtctatacatgttttatagatggttggataatacttggatagattacatatatagataaatatcctgtacaaatttgaaatggagacacccacatgtatcttgatgattcccttgaatatttatgtataccttgagaaaaatatcaagctcaaattagaatacattgcattttaattcaaattcaaaaatgctggctggttaaaaaatctaaagagattacattttatctaaatgtcaacacttcataattaattccatcattgaaattcttttgttggatttccagttctaataaacatatttttgaagaatatgccttattgtttttataactaatatagtcactttattgaggacagtggtagatatttcttaagtttaactatattgggtgtatatctgaattggtttggaatcagccagacgtgccaagattttgtattgctgatacatacacatttaaatatagtatgttattttctgcagcatctagagtgtctgcctttacatatatatagactgaaggttggatatccaatggggaacatgatgtaaaatgtggcatgatgaaaggaatgatgttccttttttttttttttttttaaggaagcttcctacctttctttttctttttttaaatttccaattcttgggagcgaattcttagtagggcttttaaatagacttccatagtctttgcatagcattaagtcactcatggtgagtgtgaatttgctcatgagcatattaattagctagtgtgtgctccattttcctcatctgtgtattaactggagtgctatttcagatatgagcaagaattgtagacctggaagagacacaggtgtaaaatctaaatcaaatttctacttttacaggtgtggtatagacatagggagtttaaatcatttgtctatggtcactagattggcttgtagcagagcctgagctcctgacattcagactgttgctttttcctatattgttaaaattatattgtggataaagaggattacttcctggatattatagggtttcactgaatctaggctgtctaaattaatacacagtctttgttgccacagtaatgctatttttctgatatttaggccctatccacacattctttctacaccagcagctcaaacaatgtctgcctatgcaggccagactcagtattcggggatgcagcagccagccgtctacacagcctactcacagacaggacagccctacagcttgcccacttacggtatttcacatcttctgttttcttctttggttataggcaggtaatcctgctggctggtagctttgtattctattgtaggttacaacttagaaggaaaatcatcttactaattaaatggtttacacattcaattttctgaacagatttgggtgtgatgttgccagccatcaagacagagagtggactttcccaaactcagtccccattacagagtggctgcctcagttacagcccagggttctctaccccacagccaggccagacaccttattcttaccaaatgccaggtaagtagctacacatgaaacatgttttgggagaactaattctttgagtgagactcattggcttcagactaggaatatccaatcatgaaggtagttttaagctttatcacagctcacacaatttctaaagaaagataactagacagaaacacgatgaaatggttgagtatacgtcaggaaacatgacctataccaaacttaaatacttaagtttaagctgcgtaacatcagcaggaaaccaagtccaagcaaagcaactcccaagccagacaccaggtcagccacacatttggcgtctacttaagcaatttaaaagatctgtagcacaaaccagtattttcttttcactcttcctgaagtatgtaatgctgttttgattatacttaaacttgaattaactcttttctaagttccatatattattgaccagttgaggattttttgttcattcctaaaattcctctctcctattgatactatattgcttaataggactgttatataatatttactctccaatcctccatataggcatataggatcattaaatttttttaaattttagactcctattaaaattgtgttttatctttttttttttttttttttttctgagatgcagtctcgctctgttgcccaggctggagtgcaatggcatgatcttggctaacttgcaacctccacctcctgggttcaagcgattctcctgcctcagcctcctgagtagctgggattacaggtgtgcaccaccacacccagctaatttttgtatttttagtagtgacggggtttcaccatgttgatcaggctggtctcaaactcctgatctcaggtgacccgcccgccacggcctcccaaagtactgggattacaggcgtgagccaccatgcctagctgtgttttatcttaatgcaatagttgccaaaagaattttagtgacatagtaaaaactttaaaagctggttactatctgtcattttgtctgtactgcaaatcacaaatttggggctatattaacatatttgaaccaactggtatgacattttggtcaattgattcaaatgaaataattacttagatatgttcagctggaatagcagtgctcttttttacctagaatgtaggttgaaacatatttgaaagcatgctatagccacaaaaattcatacagcttcagttagccatgtgacagacagtatttttttttttttcacgaatactttcatactggttattccaggtcaggtcagtggcactgattacataatacattgctagtcaaaacacattgataaaacaagtaatcagctacctcaatagcctttcattttacttcttgacaagcacattccataaggactttttttttaggaaacctgtaattggcagctcacttgtttttgccctgatttgagaaatcattaaatttcagaccttgtagtaaagttttagaaggccctcaaaccctagatttgtgtaccataaacagttctcccttgagtctgaaccctaattcacagctatttttgttctaaagaaaagacaaagggaacaaaagtacaaggagttgctaaatggggttctgtatgtttatttcccacgctaaaaaccctgtacaaatgttgaactcaagtgattttttgtatgtaaatgcagttgatatggtttgaaagttccagtagtttcctggaggtagtcttagtctgtgtaagattttatcctttgcatacccctgcacttctagactcttaacccttgaatgttggcctcactccttatgaagactgttcccatgtgcttctgtttagctcaacagattcaataggaaagggtttaccagctgttacaagagggagagagctaagcctattttctgtataaatgtctcaacttcaaatttcactgtcttcacgacaaatttcaaagtgtttctctcacagaattctgcaaagtgaaatatctttatcattttacaaggaatacttttaaaatgcaatttgttttttacctctaggttctagttttgcaccatcatctactatttatgcaaataattcagtttccaattcaacgaatttcagtggttcacaacaggtatagtagctttttgtgtttatgctttttatatgtatttcagatttttgaagagtactctcaggttgccatatatgtgcatactctcccagaataaggtttctttatttaaaaattatgatagcatttcaggatagtaaaaaattagctatctagaattcttggaatatgcattattctggctattttagattttttgattgctaaaggtttgtgctttaaacatttatgctgtagttgttttaatcatttggcatggaattacaaaatgtattggatattttgctatcttaaaatatactaagaacaattttatttttcttgataaagcagttattataatgaatattaattggttgaatcaatattaattgattaaaataagtcataggattatactacagactatataacattttcagtcctaaaaagtgtcctttgttactaaacaaaatgcctcaaagactactgttggcttttaaataatttttctgttttttagttttatgaatacttttaataaatgtttgattcccactttcctgacagctgcaactttttgcttttgtcactgtgcctgcctctggtagtctttagtcctccttctaattatgttttcttaatatttttgtgagttttaaaatctagcatgttagaattatgcatgaagcaagagtgaatgtccctgaataagtaaatcagctcggagtgagatgaattcgtgatatatgttgacacttccaaatcttttgtccttattttttgattttcagccatgttcgaagtagttgagattgaagagttagtggctgacttctggtctggcaatacactattttactttagtatagttgctagacacttatatattctaaaatgtaagttactgtacaagtgcttactttttagatttactgaaggaattttatcaattgctttattttagttttattgatatcacaaaaggcagaaaggaaattttgatcattaaaatcgtgtggaataataatatttttgataggattaaacggtgtaatttagcattaggaaaatgatgtcttaaaacactaaacatcagagccagtgtatacattttattttttaaaattgaggttttttttgtaacttgattttatgcatttatttattggtccagcaagcatttattgagcacttgctatatgttagatttttggctagggattgaagattcaaaacgagtaactcattgttccctgctctttagaaggattcagtcctccgaaagaaacaaatatgaaaataagatggacagagggtgggatgggatatagtgaatataataacagaaatatacactaggcagagttgtactataaaggaaaaactgctctgcctctatttgggaggattaagaaaagatttgtaaaggaaaagagcatttatttgaaaagagttaatcatagaacagatctgattctctgtgaagatttaaatatagtggatcccaatgtaaaatgtaatctgtatttgggaccatggcctgagcaccagtttgttgttttggctcatacctattgagtgcccataaagtgttaggaattggtctgtatactggggcacacgaaggtggggctggagtagatcagatctgattcctgcctcatagagcttacagcctactgggacaaacatacaataaaccagtaaacacacaaataaatgcagaattagaaagagaacagggcctgcgaaaacatcaactgaatataattttgaataggggaggttttatataaggcttctctatggaaaccacatttaggccaagacttctaagagttaggtgaagaaggagggcagtggtgtttcaggcaaagaaagcagagtgtaccaaatccccaatggatggaaaacttggtgtgtcactgaacttaaaaaaaaaaaaaaggctggagttcagttagtgcatgagggggaagtggggaagcacatttgcagagataggcgggggcctgctcacgtaaggcctggtggtttacaataaggaatttggattctattccaagcccagtgggaggctatgatagcattttgagcaagaaaattacatcatttaatttatgttttgagacgataactctgccaccaaggtctttggagaaagtcagaaatggaaacgaggagataaatttaaaaatgaatacattgcctgcaacaggaagaaaaggtagtttggacaagggggcgacagtggaaatgaaaagaaagtgagatctttgagatatgtttgaatatgaaaagacattatattccccagtggggaatagcctggacctttccagtatgtaacctcatcctcttctgcaggaattagtaggctaacagagagcttcagcacagatgtttgacttaggcactctgggtttaaattctagctctgaaacttactagctgtgtgactttgtgcaaatcacttaaccactctgtgccttagtccccttcaaagtaaaatgaatatagtattaagaccacataagagagtataaggattaaatgagtaaatatatctgagcccttagaacagtgctttgatacagagtaagcactatgtaaacatttgctaccactgttttactctgagagatattggtaggaactttgtttaaatatgatgaggtttttgttcttggctcacaagttacagtagtgggaaacattggatcttgaaattttcagactaatagaattttagttttctgccatattacttaatcatttatgaagatatacatatttacttataaagattatttcttttattttttggaaaccacatcaaatgtagatcttttttcctttggaacacatttcatcaggggatcttaatgttatcagtgatgaccaaggatttgtcatcactgataacattttgggctgacacatgctagatatcttaaacaccaattatcacaacaaatccttactcatcatcctactgtgtaaaaggattttgaatcattttaaggttcttaaaatttttaaataaaaagcaggtcaggggtgttgattcagtgctttcatttttctctgtaacaaaggagggaaactaaatcattgaaaagttcactaaagactttaaatcaaggcctcaggtggcatgtagtttgtgagaggctgggtaaaaaaaaaataataatattcatcttcttttatttactttttacctcctgccaagaaaacagaattatcagtggtagcccagaaaggaacaaaaatcacaaaaatgcagactgaggtctctgtggagtctaggaggggcagccccaactccttgctggtgatgttagaagatgtgtaagaacggattttgtgaatatgagctgattccatctccttctttcctcttcaaacacgtaggtcctgatgatgtaaatccttgaataagtaactatcaggagcaaagtgaagcatatgctaactttaatcacattaaaatttgtgtactggacctggtttgccaaaacaatagggtatttcactactatatgtttgtaagaaatgcccacaaagtagtctctgctcattaggatcacctcaaagctgtggactcagaaacaaatggggctgagtactaatctttcagttaccataatgactggttttcttgggagagtattaaagagtaatttctctttcaaacacacacatctcaattattgtttcaggattatccatcctatacagcctttggccaaaaccagtatgcacagtattattcagcatcaacgtatggagcgtatatgacatcgaataacacagccgatggcacaccctcttcaacctctacttatcagttgcaggaatctctcccaggactgactaaccaaccaggtacagatcttcacccaggtgaaatacttttatatgttttgcaatatctaataaaacggagaaagtggacattccaaaaacatggcggaaagctcccagataattgtgctgatgtttaatctgtgttagcatgactgtgtaagacgaggctcttctggcatttcagaagatccttcttttagatatgggacagataggcgattttaatctctaaatgatggtgtacatatgaatatggagcttttgccagactcattattcatttgtgaagaacttgcaacaaagttgtaatattatcttccttatgactgatattatttcttttgtgctaaagagactgcataagcccttcacctttgtaagttgtcagtagctctagcccgtgaacatttcctacattcctgggatttgtagaatcagtgataggtagtttaatatctttgtgtgatcagtgtgtatttaaaatccattgtgtgtttcattatctgaaacttgcagtatttgaattgcatttgcgacacgtaatttgacacagctgaagtatttaaagttaaaagcaaatgtaacagagactcagtataatcctaagaaaagttccaggactttttggagaaattattgaggtgattctaaaatcacaaggaaaggtggatgaattagaatagtcaaaactatttttaaaaataagaacgaagtttgaagactcagattacctgatttaaagacttataatgtacagtaatcaagagagtgcaaaaggatagacacatagagcaatgaaacacaatggagagtccagaaacagatgcatatatatgctagaaaatggatttttgaatcaggatacaaaggcaattgagtagagaatgaatagtttttttcaacaaatggtgctagaacaattggacatctatatgcaaaagaggaaaggatctcaacttatatgcaaaaattaactccaaatggattgtaagcctacatctaaaaagctaaaactataaaacttctagaagaaaaaaatggtggaaatctttgtgaccctgggctacataaagattttatagatatgacattaaaaacacaattcatacaagaaaagaacagataaaattagatttcaaaaaattgtaaagaacttttgctcttcaaaagacactattagagaagtgtctgttcatgtccttcgcccactttttgatggggttgtttgttttcttcttgtaaatttgtttgagttcattgtagattctggatattagccctttgtcagatgagtaggttgcaaaaattttctcccatgttgtaggttgcctgttcactctgatggtagtttcttttgctgtgcagaagctctttagtttaattagatcccatttgtcaatgttggcttttgttgccattgcttttggtgttttggacatgaagtccttgcccacgcctatgtcctgaatggtaatgcctaggttttcttctagggtttttatggttttaggtctaacgtttaaatctttaatccatcttgaattgatttttgtataaggtgtaaggaagggatccagtttcagctttctacatatggctagccagttttcccagcaccatttattaaatagggaatcctttccccattgcttgtttttctcaggtttgtcaaagatcagatagttgtagataggcggcattatttctgagggctctgttctgttccattgatctatatctctgttttggtaccagtaccatgctgttttggttactgtagccttgtagtatagtttgaagtcaggtagcgtgatgcctccagctttgttcttttggcttaggagtgacttggcaatgcgggctcttttttggttccatatgaactttaaagtagttttttccaattctgtgaagaaagtcattggtagcttgatggggatggcattgaatctataaattaccttgggcagtatggccattttcacgatattgattcttcctacccatgagcatggaatgttcttccatttgtttgtgtcctcttttatttcattgagcagtggtttgtagttctccttgaagaggtccttcacatcccttgtaagttggattcctaggtattttattctctttgaagcaattgtgaatgggagttcacccatgatttggctctctgtctgtctgttgttggtgtataagaatgcttgtgatttttgtacattgattttgtatcctgagactttgctgaagttgcttatcagcttaaggagattttgggctgagacgatggggttctctagataaacaatcatgtcgtctgcaaacagggacaatttgacttcctcttttcctaattgaataccctttatttccttctcctgcctgattgccctggccagaacttccaacactatgttgaataggagcggtgagagagggcatccctgtcttgtgccagttttcaaagggaatgcttccagtttttgcccattcagtatgatattggctatgggtttgtcatagatagctcttattattttgagatacgtcccatcaatacctaatttattgagagtttttagcatgaagggttgttgaattttgtcaaaggctttttctgcatctattgagataatcatgtggtttttgtctttggctctgtttatatgctggattacatttattgatttgcgtatattgaaccagccttgcatcccagggatgaagcccacttgatcatggtggataagctttttgatgtgctgctggattcggtttgccagtattttattgaggatttttgcatcaatgttcatcaaggatattggtctacaattctcttttttggttgtgtctctgcccggctttggtatcagaatgatgctggcctcataaaatgagttagggaggattccctctttttctattgattggaatagtttcagaaggaatggtaccagttcctccttgtacctctggtagaattcggctgtgaatccatctggtcctggactctttttggttggtaaactattgattattgccacaatttcagagcctgttattggtctattcagagattcaacttcttcctggtttagtcttgggagagtgtatgtgtcgaggaatgtatccatttcttctagattttctagtttatttgcgtagaggtgtttgtagtattctctgatggtagtttgtatttctgtgggatcggtggtgatatcccctttatcattttttattgtgtctatttgattcttctctctttttttctttattagtcttgctagcggtctatcaattttgttgatcctttcaaaaaaccagctcctggattcattgatttttttgaagggttttttgtgtctctatttccttcagttctgctctgattttagttatttcttgccttctgctagcttttgaatgtgtttgctcttgcttttctagttcttttaattgtgatgttagggtgtcaattttggatctttcctgctttctcttgtaggcatttagtgctataaatttccctctacacactgctttgaatgcgtcccagagattctggtatgtggtgtctttgttctcgttggtttcgaagaacatctttatttctgccttcatttcgttatgtacccagtagtcattcaggagcaggttgttcagtttccatgtagttgagtggctttgagtgagattcttaatcctgagttctagtttgattgcactgtggtctgagagatagtctgttataatttctgttcttttacatttgctgaggagagctttacttccaagtatgtggtcaattttggaataggtgtggtgtggtgctgaaaaaaatgtatattctgttgatttggggtggagagttctgtagatgtctattaggtctgcttggtgcagagctgagttcaattcctgggtatccttgttgactttctgtctcgttgatctgtctaatgttgacagtggggtgttaaagtctcccattattaatgtgtgggagtctaagtctctttgtaggtcactgaggacttgctttatgaatctgggtgctcctgtattgggtgcataaatatttaggatagttagctcctcttgttgaattgatccctttaccattatgtaatggccttctttgtctcttttgatctttgttggtttaaagtctgttttatcagagactaggattgcaacccctgcctttttttgttttccattggcttggtagatcttcctccatccttttaaaaagacaagctatagacttggagaaaatatttataatacacatactgatacaggaattatataaatatataaagaactgtcaaaattcactaagaaaaattaggttaagaaatttttaaaatatcattaatttgttcaagtatatttttctgccttcagttatttgcaagatccaaattcatctctacttctttcttattcagctttggatgggagggaaaatagggaaacatggtcacagtactgtaaactctttctaactggaccagtggggccaagatgactcaatgccattaaacgattatttattgagtatatattaggcatcagcactgtgctagaaactgatatttcagcagtaaatgagctctttgcccacctttggctttcatttgacaagcaggacaaatatcaagcaagtaattacaatagtgagaaagggaaaatggtaggtcatatgggggatattaacaagtaaactaatttggacataaagtaaaaatgaaactgatattttaagcaacagccaagatatgtttatatcatgaattgatgaaatgataacattcatgatttttagttccctgttcagaatataatctttacctgtgggtccaagaaataattatctagtagatagagcttgtctttgatgcccctacattttagttttttccccttatcttccttttatatcctcctacctgtgggcacacagcccctttgaatttctggtccttgtccctttggggctctctgttttggcttttcagctagccttagcctccctgggtatctgtaccccttgcagggccaggattagggtgaggtcagtgaagcacctagagcacaaaatttaaggtggaactcgttcttggggtccttaaattttgtgccctaggcgccttactcattagccccagcccaaatcacatgttccaaatctgtcatgatcctctttgggtcaatacttgtgttgcatcaaggatcttactttcattcaaagaatgaatgatttcagaatccttgcttaacaagcagtggagatacttcagggaaggagatatttcacgacctcttatcaccctgtggaaggtttttctcaatgatgaatgtgtgaggaaaacaaggtagggtgaaaccacagaagtatttaattatcaagatatcagagactgaatgatggacaagaccaaaagggtgtttttttaatttgctagactacagctaccaagaatgaaacccatttgtgtactcagatacggtatatgctacgtggagctttgtgtaaaatgttgcttttgctccaatttccatataatcacgagaaagcttctctgcaggaaaaacacagatggttactttccttattagaataatcagtattgaaaatagctattttattcctattaaatatattaaatgcaggcaggtagatattctgagtatgtgactttggatgtgacacattcacatatggaattctgacattcactatcagaatgctgcaatttcatcaggccttcattggttgctgaggtcaagaaatatagtttgacatcattttcagattttattagactgtctgttgaatttaaagaaggaattaattctacttataactatattattctctcaaccaaaatcgtaacaacacacatgtctggatctttaaagaattctatgacctcagaccttcagtcaatgcagttttaggtttaattttgccttttcagatctggcatacggccaaaaagcaaacaagtttataactatggaatttttctttattattgatggaatctgatgtatttagttcaagggcaagaaaagtatttcccattgactggttctgcattttggacttaaagtagaaacctaaaattaaacccacagtttttcatagaacagtcactgctgaattctacttttataatgaaaacataccttatgttcaataagagacagctggtttttctgatttagagataagcctaatgaaataaagtgggatctaaatatccaaaacttaaaaggtgcagttattcattactgtactgattaaaggataattattcaatcatgtatagtattgggtcaagaaaagcctagggcaattttgtaatgactgatgttacttggaaattgcaaatagcattccattatttaatttaccttattaggtagtttgacagtggatttaaagaaacactaccatgctgattaagagcagacattttacttcattctctttctaaaggtctttctttaaagtaaaatttatattgggtatatcagattttttatttgtattaagaaagtttgaggacagatttgcacatgaagatgagaaatatttttatagcgcctccttttcaaatattttaaacatctttaaaatcaaatacagttagtcttgtataacaatattatgttttaccctgacactttgacaaacattacaatacattagctaactgaacctaacaggtgctctagaaaagtaaaggatagagtaagatcatttcactctctgaaaagaagccagtttcttaggcagaacacagcagccttttgataacttcatggtatttcacgtaaggaaaggaagggaagtaaagtgctaactcatctaatggaaactaacaagaggatttagatagaaaggaccacattcaactgctctgccaatttacattttccctacctgtggcattgctatgcccttgtgcaattacctgagtgaaatttggcagaggcactggaattaacagctttcttcttttaaaaaaaagtgtcaacaaatgtttaatgactggttgagctcaagttcctggttttaccctttatcagaagaaaggatagcacctgcaaaaggaatagaggtcagtacaattccacctgggtgattgctctaggagcccagatgtatcgcatggcctctgcagggcatgctcttttgcttctgtgaagttctttcagtgaattctttggccctccatctcacagaaattatataatgcccatgtgacttttattttaatttattgtttaactgacaaataaaaaagtatatatattggtgcagtgatgggcacccgtaatctcagctactctggaggctgaggcgagaagattgcttgagtccaggagtctgagtctttaaaaagaaattgtatctatttttgtgtataacccattgttttgaaatacatttatattgtgaaatggctaaattgagctgattaacatatgcattacctcacatgcttatcattttgtggtgagaaccccgaaaatctattctcttagcaatttttagaaatacaatacattgttactacgtatagtaacatgttgtataatagatatcttgaacttactcctcttgtctagctgaagtgttatatcctttgaccaacagctccccagtctccctctccagccccacagcccttggcaaccaccatcttactcacttcttctacgagttcaacttttttgcactccacatataaatgaggtcatgtagtatttgtctttctgtgcttagcttatttcacttaacataatgttctctaagttcatccttgttgtctcaaatgacaggattttcttttttaaggccaaacattattccataatgtacatataccacatttcctttgtccgtttatccattgatgaacacttaggttgactccatatcttggctatttgtgaataatgctgcaatgaacatggaagtgcagatatctcttcaacatactgatttcatttcctttgatatatacccagcggtgtgattgctggatcatatggtagttccgtttttaggaacttccatagcattttctataatggctgatatggtttggtggtgtccccatccaaaatctcatcttgaattgtaatccccctaatgcccatgtgtcaagggagagaccaggtggaggtaattgaatcaggggggcagtttcctccatgctgttctcgtgatagtgagtgagttctcatgaaatctgacagttttatacatgtttggtagttccttctgctttccttctccttcctgccaccttgtgaagaaggtgccttgcttccccttccccttctgccatgattgtaagtttcctgaggcttcctcagccatgctcaactgagtcagctaaacctctttcctttgtaaattacccagtctcgagtagttctttatagcgatgtgaaaatggactaaatggctgtgctaatttacacccccaccagcagtgtgcaagatttctgtccgccacagtgtctcctacacttgttatttttcttcttttagataataccattttaacaggtgtgaggtggtatctcattgtagttttaatttgcatttctttgataatgagtcatgttgagcatttttcatacacctgttgtcatttttatgtcttcttttgagtgatgatcatgccaacccattgcccattttttatttgagttatttgttttttactattgagttgtttaagttccttgtgtattttggatattaaccctttatcagatgtatggtttgcaaatattttctctcactctagtaggttgttatatgactcttttataagtagaagcagatctaggtttgaccttctggtaacctgaagagtccattgctatttgcctgagaaagtatagccaaatggttaaaggaggagttctgcagtcataccagtgttgcatcatacaagctatttaattttgttaaattaattgccacttctccagtttctccattattaaaagacaataatgaaggacctaattcaccgggttggagtgaggtttaagtcagacacacacacacacacacacgcatatatatatatacacacataaatatatataatgtttcataataaagagctttgacaaatcaataagaaaaagacaaacagcctagttttcttaatgggcaaaagatttaacagttacttcattaaaaagaagatatcaaatgagcaagaaacatgaaaaagtattcttcataattactcatcaagaaaattataattaaaaccacagtgagataccatcaatcacacacccaaaatggtgaaaataaaaaaacgctaacaatatcaagtattgacaaggatgtggaacaactgaaactctcattgtggtaagagtaaattaatactgtctttaaattaataaaaatgagcagtgggttagcatggtcgttaatacactggaaagagagtaatacactggaaaaaagtcagtgtctgttaaagatgaacatatgccttccctctgacccaggaatcacattgccaggtatgcatacccaagaagaattaagagcttgtatccacaaagacatgtacaagaatgtatatagcagctttattcatagtagcctaaaattgtgaataacccaaatgtccatcaacagaatggatacagtgtaatagatacagtgtaatatatatatacatgaagaagttctagccacattaaggaaaggacaactgctatcccaggctagaatatggattatctcacagcagtgaatgaagccaaacacatatatttcaataaaaaaaatttttaaagaacagtgcatgacatgtactaaggggtgaataaatgtagagttacccttcatagttttctaaaaaggctgtctttatccccatcaaaacacttctgcagatattttgtccaaattcttagagcccttttaagatccttcagtaatttgaaaagcatcaatgggaataaagtcttaggtttactgaaacccagatacgtcctacaaccacagtcttttctctttcctcttctgctcttttcacccctttgctgtgacagctgctggatcccagttcatttgaatagcagaattattcctgctaaactcctcctttatggactcagcctacaaccacccatgactggggttcctcaagtttttgtcctttcagctcttgcactgtgacaagaacaaagttgccttatgatagttaacacctgaaaattgatctataatgactataaggagaagttttatggaacgtcatccaaaatgttttttaaatgcatgcttaataagtgagggttaattttaatcaccattttatttgaaagcagacatttggtttgtgggatatagtaaccttaggaaaattctgtttcaaaaataattaaaaatggaacttttatgtgattttatttaaaagttggtgtatgttcagacatctaaagactaatttaatgcctatacaaataaggtgaaacatgatttactatgagacaaggctatattcaaccccataacttaaaaaccttattactctgctatctactctcaccgaggtgataacatagacccatgaaatgaactatgctggcaatattttaagtgcaaataatatttggagcacaggcatatttttttgatcatgtgctgacatgcacagtgtttcccaaagaaaatgttagaggttacccatgtaaaaattactatttctcagaaaaagtgatgcctagtttgttcagatttgtttatttagattaataactgcttggttttcctcagaattgttttattttaatttttaaatttaatgataaaaaataatagccataatatctttttgtttaaaaatcatgtaatttaaatttaatgtagtttaccttctgtcttggaaattgtcactttatacatataatttaaatcaaattaaatataaatcagatttatccctcagtattccagaaagctgccatgaaactgagatttagagagattgaataaaatgcttgtactcatatagtaattctgactcttcaacttgttatactgtatctcacagttgcctcagtttatcttaacagttgctcctacagatttgctactactcagtactctctacctcctaagatactttctcacaagctttaatcatttcagagttttagatatctatgggacgtacagaagtcaattggattctgaaactgaattaaagtctgaaacttaggatgtggcctagttggaggtataggtttatatgtgagtcatcacagagagggtcattaacacatgagagagttaaatcccccagattgtgtggagtgaagacagtgatcagaggatggaattctgacatactagctcataaggaatgggagaagtacaggagtccatgttttctagatggagatggactgatcggagtgattctagaactaggagagtgcaaggtatcagctgtcaaataatagtttcgaggaggtgctgtttaagagaatcacacacagcacagagatcctggaagacaaggactgagtctgtctattgcatttgtcaacaaggtgatttctggggactattagcaggagtatttttatcgcattttggggaactgaagctagattatgataaattggatatcaataagtggggacagatatgccgtccagtgctgcagccactggccacacgtgactcttgagcacttgtcatgttgctagtgtggactgagatccagtataagcatagaacactgcacaacatttcaaagacttagtatgaaaaagaaaatgtaaaacatatcattgataattttatattgattacatacggatataacatgaatatattgagtcaattaacacattgattacatactgatatgataacatggatatattggggcaattaacatagaagtgatgataattttacatatctctttttactttttttaacatagctgataaaaaactttaaaatattatataagatcacatttgtggctcacatggtatttctgtcatatagcactagtatgcactattctaagaagttttatgtaaaagagaaaatggaagcaaatagtatctagatggaaattccaggtcagagggaaggactttttaacttctcagtgaccccaagaaatgggagaggtggaagggaaaatgggagaggtggaagggaagatgggagaggtggaagggaagatgggagaggtggaagggaagatgagagaggtggaagggaagatgagagagggtttgactaatgaagcatgacttcctaggaggtggggctgggagtcaggtttgcaggtggagggatggaattgatccagaagaagacatctcattctctgaactgtgacaaaaaaggaggtgaggacatatctaaatggagataaatatgtaagtgaaggtatagacaggaagttgaaggcgtatggtgtagaaaagacattcaagtttaatagatcacatagattacaactagtaggagtttgtgtaaactacaaaagttctgactgacttgtgtgtgcttgggattataaatctcttttttgccatcaggaggtttctattgtataggaatttgttaagataattaataatgaagactcatacttgatctaaaaatgaagtgctattcttgacctaagtcatgttatctataggagagttcgataccatgcagagtccctccacacccatcaaagatcttgatgagagaacctgtaggagttctgggtcaaagtccagaggaagaggccggaaaaataatccctccccgcctcctgatagtgacctggaggtatgcctactcattcttaaagattgtagtgtgatgctttattttaccgaatgatacattctctatcttacagttccattatgtttcaaatttaaaaatcaagatatatcatgaattgaatggaactactttgtgataggcattttacaactctgaagtttgagattccatccattataatattaaaattaagaacaaataacagtataaaaatgatcttgatttacagattattggcctgagataattgcaagttttgatctaaattggcaccgacaaattttaaaactatagccattgtttgtgggatacttttctatgtagcactgtattcgatagcactaaagtaatggaagatgtccaaatgggtcattaagtttaaaaagatgaaagtaatttgtatagaaagaaaaattcatcttctaaaaataggacaagtttcaatcacttaatagtatgtgttaaaatctgaaaatttcattatatgtcactttctatttatgagttcttttgagatatatctgtactaagcagggttgatttttatatacaacatcagtagaaactattcttaaccataatatgttacttcttacataccacatacttcggttaagaaaaaaattgaacttccagatttagtaaaatcaagtgacagacgttgtgattctttataagagataacttccttttcttgaacaacagcaacaagttcacattgcctcaagaacttgacagatgatataaatgagtgaccgggctgagttgcccagcagactgtgaggagctcagagtgcaggcccattctgagggacctgcctgtcactgccatgtaggaatggcagccagcctggccacaacttctagttgctgtgctacagtcaaaaccctgaagtttgtgggcaattgcctagttctcaaatgttaattcataatttgaaatccttcaaaactccagggaggccaacatatctgctggcctaatgtgaccccaggatctatacttgacaattctgtttagcggttactcattgattcttttaaatatttctttaaaatctaattctttgcagaaattgtaaaagtttatgaatccctaaggtataaaactttaaaatatgtagagtttcaagcatgcttctatcataaagtctgaatgtatctctttttggtgttaactacgagtaaatgtatggattaatttattcaagcgaggacatctgactcttaagcttataatttgtataattcctgctcctcaggctttataggagttagatttggcaaatttgaaaaacaaaaaaatgaatagtattattttctattaaatgatatcaagatgatctctaggaagggagacattttctgcttgtaaagagatttctgtttccttggactttttaattttctgatatttattttttgtcttcagcgtgtgtttgtctgggatttggatgaaaccatcattgtttttcactcactgctcaccgggtcttatgcacagaagtatggcaaggtaagaaatcaagaaatgttactccaagaaatcttgttaaattttttgtgtttgtttaaaatcaagggctatttaaaaattcttttaagtgtttttttattgcagtctgtgaaatcttctcttaattatctatttctagtgtcttatagttctttaatatgaactggcaaatttaacaaagctaattataaattgaattccttgctatttcttcaaaaggactgagttttctacatcaagaccaagcttcttagtctcacataattattttagaaaaaaaattgtaaatcgaaatggacttcaaaacattataaaatcatatattatccccaataaaagagctttaagaaattgtttaaattaatttgttgcaattttatagcaatagcgagtaatctttcgtctgacagatcttttggatttatgaatgagtgttttttctgtgacttgagtacacatatgaacagcaaccgaggtaacaccccctcccatacacaaagtcagcaatagatggtctcatgcctcacctgcctttctctcttgctattgtttttattttctcttttatttatttatttcattgttattattattattattattattattattattattattattttgagacaaagtctcactctgtcccccaggctagagtgcagtgacatgatcttggctcactgcaacctctgcttcccgtgctcaaatgatccacctgcctcagcctctcgagtagctgggatcacaggtgcccgccaccatgcctggctaatttttgtatttttagtagagacggggtttcaccatgttggccaggctggtctcgaactcctgacctcaagtgatctgcctgcccctcggcctccaaagtgctgggattacaggcgtgagccaccacacccagcagttatttttatagttagcttcttataacatcattattacaaacctctgtgagtgcttctccgaagactgatgcaactctgaaaggctttttggctagaaacacagctgtttaggcaataactgtttaagtcttagactctgtagggctcataatcaaactaaagccaactcagaacatgttggcttactgtcagttactatactcaaagttgtatctttggttcctttacatatcacgcttacagaaatgtcttcctatgtcttcatagcattccattattttataatatctcatgctcatgcagtttttagctttgtctatattaagctatattaagctttgtcttctgacaagtaatgataaaattgcgttgaagtctagtcttcctaaattacactttgggtgaatctgcattgtcatttggttttagtataggtaatgaggtaatggatatttctaaacagatgtgaaggtaataattttgtttaacaaccatttacaagaagtgttagcccctttatcacaaaagtaaagtaaattatacagaaatttggagtacacactcaatatcctactactgattaagtgtgcaatataaaaagtttccagactataatctgaagaatttattctaagtagtattggcttatcagaagtacacagtaaatcctgttgagctgattgcaatatttttgtgcagctaagtttcatattaggtaagtgtttaaaataaacaatctaataagcagactggcccaaataaagatcccttcaaaatagtcaccatgaggtgttacacctaaatatccatgttgcccccattgtgccaaatcagtggtagaactcatcttttggaattatcattagccttttaacatatttcctggcttcacatctttatccttttgtggtcattttgatctttctttaaaaatagaaagatcattcggacccaaggcggtgaataaggagcatggtaaaactgtcactgtatatatttgttttatttttgtcaaaaataccatagagtaatgaagctgcctttctcaatttactcataaactggctttaagagcaattccaaaagcaaagttcaacttctctcagcactgctgcgtcatggaaataaacctgtcagctcccaggatgtcgacaggacatgcagcactcatttcaacacacaagttttatttttccaaagtatttccctaaaaggttttttttcaaattcaaaatagagactctctatgcatcctctgtgcttctagaaggttaaaaccaaaagaaggagaataccaaaagggtggggcaaagcgaggggaagaaggaaactaaccattatggaaggctcatgatgcgccaggcaggaagcatcccacaggatgaaggggtaggctgttctctcaaagctatagatgaggaaacatatccagagaagtaataaaaatgtgtccacagccacacaaccagtcagtggcagagacgagtcctggggccccagattctttgaccctgaagccatgttctttctgccatagcacatcgcatggcctggacagttaatataccaggaatgcaatagaaaagtaatatttccaaacaggagtaaagacaattagtgaggtactgcataggcagttgggtgagttctcttttaagttgtaattttaatgatcttctctcagacacttggaatgtgttttcatcaagaggtaaaacttaagaaatattccatgaaatagaatatggcagacagtaatcagtatctgctggtggggcagaatacagaaagaatcagaggcactattggaaggggatgagaaagataaacttacatttataggatacctgtcctgtggttcagccgatgtttgacattttagaagcaatagggtggtggtgaaaggcaaaagcttttgagtcagataaacttaagttcgaaaatcgagttctgatgcttacagcattctgactctggcaaattacttaaatcttcagtttcaaaatctgtaaaatgggaataacaccatctgttagattgaccacactgaataatgtttttactcagagcaatttaagaaaactaaagtaaaagtgtttttaggctatattttttttaataaaaacaaaaacatccttgtcgaattccatgaaaaacaacttatattgccttatttattgaattattacatatatttctctaaaagttgttttatcattgacaatttacttaatgtaattcagaaaatcggcactgccttttagccagtaataaataatatcttggcatattgagaagtagctaaataaggcccatcttatgaagctatggaccacagacacctcagggttcacggcagatgggacagagatgacgttgggtagcttcagcttcaccacaaaccataactgaaaaatggtaaactagagcccctcagacagactgcattcatttaacaaatagttagtaatatcaatagacatgcttcttgctctcatggagcttacaaaccaaacaaaaaacaaaacaaaacaaaaaaaaacacatttcaggtattggtgacttctgtgaagaaaataaaactaggtcgtataatgagtgactggggtatgtgactgaggaagtgacattggtactgagaggagctagccctacaaattaagagagtgggagaattggaaagagcattccagcagtgggtgctgatcttccagtgttccaggaatatgaagtctagtgtggctggaaggttgtaaacaagagaaagaatagtacaaggtaaagggaaggaggtggaacaagttggcaaaatagaaacctccactgattgtcctccctgcagcaacaccaaattgaataagtatccacacaacacaaccaccttcataagaaccaaaagccaggtgagcaatcacacaatacctagttttaactcatattactgaaggaggcactgaagagggtaagaaagacagtcttgacttgctgacaccaccaccccacaccagtcttccagcagcagctgaatggcacagagagagaatctgtgcacttgggggagggagagctcagtgagtgtaggtttttgcattggaactcagtgacctttcacagcagaaagcaacactgggtagaacttagccaacacccatggagggggcatttagaccagtcctagctagagaagaatcttccatctcagctgttggaacctgagttctggcaaatcatcaccaccatgggataaagtgctctggggtccaggcagtttaggccacaaggaccgcaattcctggacaagtcctgctgctctgctgggcttgaagccagtggactcggcagacacatgacatggtaagacaccagccagggcagccaagggagtgcttgtgctacccctccctcaggcccaggcatcacagctcacagttctagtagagatgtcttccttccacttgagggaggagaggaaagagtaaagaggaatttgccttgcaacttggatactaactcagtcacagaaagaaagggcaccaggcagagtcctgaggctccagttccaggccctggttctgagataacatttctacacacgccctgaaggaagcttgcttccttgaagggaaggatccaatcttggcaagattcatcacctgctaccaagattcatcactcttgggccctgaataatcaacagtagtagcaaggcagtacttgccgcaggccttgggtgagactcagagatgtgctggctttaggtgtgacccagcatattcctagctgtggtggctatggagagggactccttttacttgagaaaaggagaaggaagaataaaggggacttcctcttgcagtttaggtaccagcttggcaacagtgggatagggcaccaagtcggcatgtggggtccctgattccaggccatggctcttggatggcatttctgcacctgccttaggtcagaggggtgcccactgccctgaagggagagccctaggcatgaaagcattcgccacaagctgactgaagagcccttgggcattgaatgaacatctgtgatagctacacattacttgccatgggcctggagcagtcaatagtggtcatgggagagaattttctgcttgtggaaagggtagggaaaagtgggaaggacttcattttgtggcttgggtgctagctcaaccacagtagaatagagcaacaggtagtttcctaatagagcaccaggtagattccagattcaggtcctagctcctagatggagccaggctctgtagacccaccagggaccaggggaacttgacaccctgaagggaatcatacaaacttggctagttttgccacctgctgattgtagagccttgagcaaacataggtggtagccaggcagtggttaccacaggctttgaatgtgacccagtcctgtgctggctacaggtctgatccagcacagtcccagggtggtggtcacagaggtgcttgtgtcaccctttccccagctccaggcagctcaacacaagagagagaccccatttgtttgggagaaagtaagggaatagaacaaaagtctctgcctggtaatccagagaattctttgagatcttatccaagaccaagaagagggcatctctatgagtctgcaagagccacagtattactgggcttgggtgcccccatttaatgcagatagcatacagtgaccaaaaacttagatcacaacacccaagttccttcagatacctcaaaagccttcccaaaaaggacaggtataaagaagcccagacagcagcgactccagtaaatacctaactcttcaatgttcagacactggtgggcatccacaatcattaagactacccaggaaaacatgacatcaccaaacaaggcaccagtgatcaatcctagagagatggagatatgtgacctttccgatagagaattcaaaatagcttttttgaggaatcgcaacaaaattcaagataacagagaaggaattcagaatcctataaaataaatttaacaaagatattgaagtaaaaatcaagcagaaattctggaattgaaaatgtaatggacatattgaagaatgcatgagtctcttaacagcagaattgataaagcagaaaaaagaattagtgagcttgaagacaggctatttgaaaatacacagccaggggagacaaaagaaaaaagaataaaaacgaatgaagcgcacctacaagatctagaaaatagcctcaaaagggcaaatctaagagttattggccttaaacaggagatggaggggtggggctagaaggtttattgaaaaggataataacagagaacttcccaaacctagagaaagatatcaatatccacatgcaagaaggttctagaacaccaataaaatttaacccaaaaaagactatctcaaagcatttgataatcaaacctcaaagcccaagaataaagaaaggatcctaaaatcagtgaaagaaaagaaatgaggaacatacagtggagctccagtacatggcagcagacttttcagtggaaaccttataggctaggagggagtggcatgacatttttaaagtgctgaaggattaaaaaaaaaaaccatatttaccctagaatagtatatccagtgacaatatccttcaaacatgaaggagaaatactttcccagacaaacaaaaactgagtgatttcatcaacactggatttatcctaccagaaatgctaaagggagttcttcaatctgaaagaaagggatgttaatgagcagtaagaactcatctgaaggtaaaaaactcacaggtaacaataagtacacaggaaaacacagaatagtgtaacactgtaattgtggtatgtaagctactcatactttaagtagaaagactaaaagatgaaccaattaaaaataataactgacaacttttcaagacatagtaaaataggatataaatagaaacaacaaatagttaaaaagcaggggaacagagtcaaagtgtagagtttttaattagttttctctttgctggtttgttagtttgttggtttatgcaatcagtgttacattgccatcaatttcaattaatgagttatattatttgcaagcctcatgataactgcaaatcaaaaacatacaacagatacaccaaaaatacaaagcaagaaattaaaacataccaacaaataagtcatctttactaaaaggaagacaagaagaaggaagaggaaaccacaaaacaaccagcaaacaaataacaaaatggcaggaataagtccttacttatcagtaataacactgaatgtaaatggactaaactctccaatgaaaagacatagagtggctgaatagattaaaaaaaaacaaaaaaaaacaaaaaaccgtgacccagcgatctcttgcctacaagaaatacacttcacctataaaggcacacagagactgaaaataatgggatggaaaaatatatttcctgaaaatgaaaactaaaaaagagctagagtagctatacttataatcatcaaagtggatttcaacacaaaaagtaaaacaaaaaaaaactataaaaattagacaaaagaaggtcatcatataataataaaggagtcaaatcagcaacaggatataacaattttaaatgtatatgcactcaatactggagcacccagatatataaagcaaatatcattaatactaaagagagagataaacttcaataccatagtcactggagacttcagcacctgactttcaacattgggcacatcatccagacacacaatcaacaaagaaacactggacttaatctgcacaggcttagaccaaatggacctaatagatatttacagaacatttaatacaacaactgcagaatatacattcttctcctacacacatggatcattctcatgatagaccacatgttaggccacaatacaagtctttaaaaattaaaaaaaatgaaattatattatgtatcttctctgaccacaatggagtaaaattagaaatcaataatgaggaattttggaaactgtgcaagcacatggaaactaaacaataactcctgaatgaccaatgggtcaatgaaaaaattaagaaggaaattaaaaatttcttgaaagaaaaaagtgataatggaaacacaaaatactaaaccctatggaatacagtgaaagcagcactaagaggaaagtttatagctataattgtctgcatcaaaaaaagaaaaacttcaaataaaccacctaatgatgcatcttaaagaactagaaaagatcagagaagaaataaatgaacttgaaatgaaaaaaatacaaaagatcaacaaaattaaaagttggttttttgaaaagataaacaaaatagacaaatctttaaccagcctagctaacaaaaaagaagactcaaataagtaaaaccagaaatgaaaaaggagacgttacaactatacttcagaaattccaagtattcttagaggctactgtgagaaactacatgccaataaactggaaaatctagatgaaatggaccaacaactagacatataaagctgtcaagattgaatcatgaagaagtccaaaacgtgaacacaccaataacaagtagagatggaagctgcaataaaaagtaccccagcaggcccggcgcagtgggttatgcctgtaatcccagcactttgggaggccgaggtgggcagatcacgaggtcaggagatcaagaccatcctagctaacatggtgaaaccccgtctctactaaaaaatacaaaaaattagccaggcatggtggcgggtgcctgtagttccagctactcgggaggctgaggcaggagaagggcgtgaacctgggtggcagagcttgcagtgagccgagttagcaccacagcactgcagcctgggggacagagtgaaactccgtctcaaaaaaaaaaaaaaaaaaaagaggaagaagaaaagaaagcctgggaccctatgactttactgctgaatttttccaaacatttaaggaagagctaatagcaaccctgcccaaactattccagaaaacagaggaaggaatatttccaaactcattctacagggctattgttactctgataccaaaaccagaagaagacacattaaagaaagaaagctacaggccaatatccttgataaatattgatgcaaaaatcctcaacaaaatactagcaaactgaattcaacaacacattgaaaagataattcatcatgacccaagtgggctttaccccagggatgcaaggatggttcaacatatgcaaatcaatcaacgtgatacatcataccaacagaatgaaacacataaagattatttcatctgatgctgaaaagtatttgataaaattcaacatcctttgctgataaaaaaaatcttcaaaaaactgggtatggaagaacaataccttaacacaataaaagccatatatgacagacctgcagctgatatcacacagaagggggaaaaattgaaaaccgttcctctaagatctggagcagaacaaagatgcccactgtcaccactgttattcagcatagtactggaagtcctagttagagcagttagacagtagaaagaaataaatggcatccaatttggaaaggaagaagtcaaattagccttatttgtagatgatataatcttatttttagaaaagcttaaagattccatcaaaaaactatcagagctgataaaatcagtaaagttgcaggatacagaatcaatcaacatacaaaaatcagaagcatttttatatgtcagctgtgaacaatctgaataagaaataaaaaagtaatgctatttataatagctacatataaaataaaattcctaggaattaaccaaagaaatgaaagatctctacaatgaaaactgtaaaacattgatggcataaattgaagaggacacaaaaaaaatggaaagagattccatgttcatggactagaagaatcaatattgttaaaatgtccgttttacctgaagcaatctatcaattcaatgcaatcactatcaaaatactactgacattcttcacagaaaaaaacaatcctaaaatttatatggaaacacagaagacccagaatagccaaagctaccctgagcaaaaggagtaaaactggaaaaatcacattacctgacttcaaattataacacaaagtgatagtaacaaaaacaacatggtactgtcataaaaacagacatataaagcaatgaaaagaatagagtacctagaaacaaattcatacatttacagtgaaatcattttttacaaagatgccaagcacatacattggggagagaacagtctcttcaataaatgatgctgggataatggagatccacgtgaagaagaacgaaactagacccctatctcttgccacatacaaaaatcaaatcaaaatggactaaagacttaaatataagatctcaaactataaaactactacaataaaacattggggacactctccaggacattggactagggaaagatttcttgagcagtaccccacaagcacaggcaaccaagcaaaaatggataaatgggaggcatcaagttaaaaagcttctgcacagcaaagaacacagtcaacaaagtgaagagataatccacagaataggagaaaatatttgtaaactatccattatacaagggattaataagcggaacatacaaggagctcaactcaataggaaaatatttaataatctgaataaaaaccggtcaagaaatttgaatagatatttctcagaagacatacacatggcaaacaggtacgtgaaaaggtgttcccatcattgatcatcagagaaactcaaaactacaatgagatatcatcttaagacacttaaaatggtttgtatccaaaagacaggcaatgccaatgaggttgtacagaaaagggaaccctcatacaccgtttgtgggaatgtaaattagtaccaccactatggagaacagtttggaggttcctcaaaaaccaaaaatagaactaccatgtgatccagcaatctcattgctaggtatatacccgacagaaaggaaatcagtatatcaaagaggtatttgcactcctatgtttatttcagcacttttcacaatagccaagatttggaagctacctaggtgtccatcaacagatgaatggtaaagaaaatgtggtacattacacagtggagtactattcagccataaaaaatgagatcctgtcatttttagcaacatggatggaactggagctcttttagtgaaataagccaggcacaaggtgacaaactttacatattcttacttatttggaagctaaaaaacgaactaatggtgacagagtagaacaatggttactagaggttgggaagggtagttgttgaggggtgcagttagtgggaatggttaatgggtacaaaaatatagttagacagaatgaataacatctagtatttgatagcacatcagggtgactgtagtcaacaataatttattgtacattttaaaataactaagagagtccaggcacagtagctcaggcctgtaatcctaacactttgggaggtgacgcaggagaatcacttgaggccaggagttggagattagcctggacaacatagtgagaccctgcctctacaaaaaaaaaaaaagtgttttttttttaattagctgggcatggtggtgtatgcctgtattctcagctactcaggaggctgatgttggaggatcatttgagctcagagattgaggctgcagtgagccatgttcacaccactgcattccagcccagatgtcagagtaaaaaataaataaaattataaaataaataaaagagtttaattggattgtctgtaacacaacaaaatgataagcacttgaggtgatggataccccatttaccctgatgtgattattatgcattgtatacttgtatcaaaatatctcatgacgccaggtgcggtggctcacgcctataatcccagcactttgggaggccgaagtgggcagatcatctgaggtcaggagttcaagaccagcctggccaacacggcaaaaccccgtctctgctaaaaataaaaaaattagccaggtgtggtggtgggcacctgtaatgccagctactcaggaggctgaggcagcagaatcgcctgaaccccagaggcagaggttgcagtgagctgagatggctccactgcattccagcctgggcaacagagtgagactctgtctcaaaaaaaagaaaaaaaatctcatgtatcccatgtgtatatacacctactatgtacccacaaaaataaaaaatactacagagatatatttatttatttataaataaatataaatatattcatttatttataaataaatataaagaaatatattcatttatttataaataaatataaagaaatatattcatttatttataaataaatataaagaaatatagaagagagaggtaggcaagggctggaccaggcaaggactcttcattatggtaaggtgtttgggttttttctggttgcagtgagaagctgttggggagttttaggtttattattgtctgctatgcagagaatagaatttagaggaataagagtggacacaagttgaccacttaggagacactatgcagtaagaatggccatgccttagataggaacagtgcttctgaaacttgagtgtgtgtcacaataacctgcagggcctgttgagcacagattgctgcgttacaacccagagtttctgattcaagaggtttgaggtgggaccataggttttgcatttggaacaaatttccagataaagctgatggtcctgggctgcattttgagaaccactagagtggagtatttcccagactttttttccttgagcaaactaatgaacatgttaattaatagtctttgagaaaaggccatggtcaaatatatttagggaatgcttacattttatagccattcaatgcatgttatcatattaaaggtcatgagatgtttaaaagaatcctctataactaagtttaatccctcagttccctaatgtcatcagtaataaccttgaggactgactgaaataaggttattctgatgattagttaagtcaggactttgcattgctaataacaaaagtgaccagagccattatttctgggaggggggaaaagacctttgtagtgttaagattgtctaagcttgaaattattatgggtccttaaaattattatgggtccttaattcttttcatttcttagatcagtcttatttccacaatatgcatgttagagtgtaacaacattttggtatagcatagtgatttttttgattattttagccagagattcctctacacacatatgaccgagagcatcactgtatagaacatctaagggcagaactgctctaggtaaaatgtacagatgggggactggagagatcccatttcccctacatgaccacccttccccatcattcttggtcaccatgaaaggtgttcctgaatcctggagcttgctcagaatacagcctggaaggcagtcgactagctgaaggagcactgaactaaaagtcagaaggacctcctgtgcctggctgtgcctaagggcaaactgacctctgagcctgtttcatctgaaaatggttataattcatttccttccaggggttacggggtggcttaaatgagataatgtacgtatataaaaatgacactacacggtgaaaactatcatctacctcctaggttagagaaaattcataaaaatataccttctttctccaaaatgagaaataactaaaggcagatgttttcagacctgcaaggtcaatgtttactcctgcaccatgtgaagtggcactgcagtggcctcaagtatagtgagctgggagctgcacctatgaagaccagagatgtcaatcatatcctgaaattaccatacctacctgcatatctacctgtctttcttcctctccccgcacccataattagtgggcaatactgagtatcatttttttactggttactgtgcgcctggcccttggctgagtgtcctgtgaaatgcattctctcattagcttttcccatcaccctgtgtgattgattggtactaccatacatagctcataagtggcaagatcagtgaaatccgtatctctttggcaggttcttaatctctgtattctttggtctcgtgttctttccccacaacttgtaaaaataaaatgtgtggaccacagtaatgtaggaatgaccttttacccataaattgtcccaactaacttttgtgttttaatttaaaaaaacttttggaaaattctacttccatttataaattaaattatgtgaatttcccaagcatcttcctaatgggttatcactttttgaccctgttcaattgtcaacactgtcttttccaagcagaactattcctggacatttctgacgatggtgaatgaggaaccttgctatctgagccctgctgctatgaggctggtgatgggttttgcagctgagcaccaattgcaatgtacagtgtctgtgctgcaaaactctaaaataggagcttgtaaattcgacaggatgtagaccaggaatcatccatcacgttagagtttatttctattccctgattgtgtatatcattttacctgataacttgcagtgattccttttgttttcaggggagggtgaagaaagctatataaattgtgctttagggatttggtttatcttggatcattaatgtatcatgtgctatgaagctatgatactctaaagttattccacagtaaggacacagaggaaatagcccaagtcagggggaaaaagtgaaggaactgcaggaaaagtgaatggttggctgataatgtgaaactcagattcagggtccccaaactgcttgcttattttagacaacatctcatattaacagcagtgctggcaaaataatatgtttatcagaattaatcaattgattgcttgtatattgaacagtctacacacatttgtgcagaaaaatacttctaacatagcatttggctggattttaaatgataggctagtcatgaagataaatgctgatgtttgtgcatttggactaaagaaaaccaacatggtttaatagaaagtaatagaattaatcaagtgttagaaccatggatctgaggcaatcctaatggcagtgctaacaaacaccatatgcttagagggctctggctttggagggagaatgaccctgagttcaaattctgcctctccctcctagtacccatgtgaccttgagtagtttatttagcatttatgagcctcagttgtcttgtatataaaataaggagaataataaaagtagtaatttgattctgagggttaaatgagttaatgcattaaaactcaccacagaataaacattcagtaagcataacctatttatttagtttcaaggaaatctggaatacaggaaactgaatttttctaaggaaatttgcattttatatttgtatctgttattttacctcataatatgagtcagtcagtgttgcagagaagtagcactatgccagggtgactagagagtggttttggtatatattatataaaaggaatgattacagaatttgcaagactcaaaaaatgcataatgtttttactggtagcttatagtaagtttgtgtgtgcatgtgtatgtatatagtgtgtacataaatatcatacatatgcatgtgaaaataaagccaaatcaaagacagtgacatatctcgaactcttccctagggaattggggaatcattgctacctattgtctcccctttcagagcttcgtgttgagtatatgtttgagaatttcttttaaaaattaaatggaatgactctgcgtgatctcatgaatagccaaatatattcctaacttttatttaagcccatctttagcaagtaattgctggaattatgttagaaaaggggaaagatcttgaaaggacatcacattgattaacttgaagctaatataccagagagataatggtaactaatctcttaaaaacacatagagcaaccttctgtaacctgccctgaagttttcagttcctttgatctcagttgatgatcatttaatctcatatcttgagaaggtgggggattagaacattaacttaccttaagtattctttaaccatcatcaaccctctcctaggcatttcctcctatagccatctcctaacatgtcaatcacctttctcctggagtcctgaggagcctcgtgaggtgtgtgtatttgccacctctgctctgccacttttctctgggccctgtgctttctagcccatcctcccaacatggtatttttctttccatccaagtaccatgttacactgttgccatgtactaccactgtaattccccccttgaagtacatttattctgttattagtttaatacatttggattcccttctgactgaaactgtagaagcctctagtggtaagttgtgtgatgatccccttcatcatacaatttaagcttgacaacattaactactgttcaactgcaaatatgttgcttacactgaacacttgaattgtgaacaaaatcttttaattcaaaatgtgctagcttttcacctttaccagctccccagcaggtaattggctacattactaagaaaaagacaccatcagacatttggtccttgaagttaagtccacagaatgatctttatggaggtgaagggtggaaaaggcactcttcctcataagcaactctagagaagtctagttcccgagaaattatactaagccacagttctactcagtgatcacgctggctcaagtccagccgtcctgtggaagcaccacagaggcttaactcttggaaagccagaaggtcatgccagctctgatttttcttaactatcccagtctctcattataatatcttatagaccagtatttattcaaaagtaacatgtatatgaatcacctgataatcttgttaaactacctggcaaactagatctggggaagggtctacaagtgtgcatttctaacagtttccagatgatgccaatggtgctggtctgtgaaccacactttgagtagcaagactgtcatctgctgactctcaatttcgtttgtacattggactcactgcagagctttagcatgactgatgcgtggacccacctcagataatccgatttagttggtatggggtgtggcccgagcaacaggcattgtgaaagctccaccggtgttccccatgcatggtggagtctgagaactcctgtcttatatgtcatcctcccagcttggagtcctccttatgtaccactataaccctacttttccataacgcctctctatcctttcctcattatttctccatcagaaaaccccactgactgcctgtacctcaacagttgaatgtggccacaaatctcaagtgggtgcaagcactcttggggtcatgaactcttcctattggaggttttgaagagctgaagggagagcaagcatatctctcccaattctgtacaaacctcctcaattactagggggaaaaaagcaaacaaatgaattctgtcatctttgcaccaccaaatccgtgcctcctacatctgtagccatagatgtctcttgcacctctcttgcaccctggttccgtgctccctcacctacccaaagaatcgactcctgaaaggacttctttcgctaagccaccagtttctcactctcctagattcttcccatcaccatcgtatctgctgtcacattcctgtcattagaagagcttcttgagcccattttcccctctggtgactgctctccttctctgctctttccagctttgccataacatgacacttgccaagtctgaggagccaccttgttgccaaatcaagtgttccttcttggcttatcttgctggggcctctcacatacattcagtggagcactcttgcattcttaaaatccttgtttcctttcccttccctggtcagggtccctcttctcacacacagactcctcacctctgggctctgtgtatgttcgctactattgcacctgttcatacctagctccctcacctttgtcaggcctctgtttctctctctgccattacctcctgaaagggggcttctctgaatttgctttaaattagccctttccactcatcctcatcacttctgtatctgcttgtcctggtttttctttcatagctcatcaccacctgaaatgcttatactcccttgttttcttgtctgtttatcagatatctcatccagtaataatataaaccacataaaggtacgggctttgcttttttggttatctgctctctgcttggaatttgaacgtgtctaatacataattagtcactcaataaatatatgatgaataaatgaatgttcctgaaaccattttccagttcctcctaagaacctgtaagcccactacctatccatcatagacaagtatcacaattgtaaaatttctgttctgatttgtgactgaatttcattttatctcttatgatttaaagagtatatgtattctaaaaaatccagtaactttttccaccttggttcatggagctggtctgccttctttcttccatgatgaacttgcatatcttctatctttgtgtgcacatgtctacacatatgctacgggtcccaaaagaataactgtttcaagaggttgatgtctttaaaaaacaaaaaaaaacgtccttagtttctttaaaaatacataattgctgcctctccagagatatttttacagtgtgtccctagaggaggaattccagctgatggcccaatgcagggcagaggcccgtagacagacagtctggctccaatttatgctctgtgttcacatggctaataggcgttagtattttgatttagagaactgctgtactggatcctgccctggttatacactggcaacatctggcttagaaccacacatttaaaggccaagagctatttattttcttcatcttcctcttctctctctctctctctctctctctctcactctctctctctctctattcagtaaaatgtctggggaatagtttcagtattttgatagagcagtacacagaataaattggcagtagttcaatttgcttcctattaaactctgttgtagacactttgaaccttttggaactgttataatatgtataatatatatcagaacttccacaaaaccctgttagtgcctttattatcccacttttttcctctatccaagattttgctatcacctcattttagaccctaaactcaacattcactacacttaaatgcaaattaaaaaaaagcaactcttgattcataaggtgcccataattctacaaacatctaatgaaaatgagcaaacatctctgtataaaacttgggttcaatccagaagtatgtttcattctcacatgcctgataaaacagtggaattgcaggtgctggcagctccctcacagcagtttggcaagatggagggtgcccagcattgctaaggctacacccaccaaatgtccagtcatctttctgggattcttggatctgtgagtcctttagctggaaaaatgcagtgctgtaatctgctcacttagaattagctgtcccagtgacttagtaagctggaaaaaaacaaaattaaaattagatatttgaaagtaagcctgccccagacattctcttagaaacaagatcaccacctcttacaggcatggttttccttggctgatggcatgactagccctggcccatctgtgccaagactttggcatggctgatgaaacctctctctttcaataggccttgctctctggaaagcttagtaaaatacagctagttcacactccagcaaaccacaaacctaaactttcccagttacaaatgatattttgccaaaatgcttactaagtcctggctctcacagagtgtgtgatgtggaatctccacagcttgaaaaacaaatgaccagccctcccagtgccgtggggttgcattccagtggagtgctcaactacagtttttatcacaccacatgccatgcttgcttgctagcagattcttgcattttgctttttttaaaaaaaattatgcttgtttgcatgcttatcagatgtacaaaatgcattcttcatggattttcctatcactgtttgggtagcacagagttacgtacttcagaagcatggaattgatcaaagtaacaatcaaaaggaacagtatgttttctcccatccccataaatctgatctgtaaatgcaaagaatgtgaagaatgaacataagaaattcaataatggtatttaaaacttaaaaagaaaaattggcaaatcataggataagagcctaaccaacctggtctccagaaggaaaaccttttaaatcagatatttctgaactcctatctgaaatatatatctttagggcagtaatcatcagaaacataacagactagtgtggagctcagttttattgtcttgcatcagtgaacattcctcagaagcacagagcgtcactaatggaattatatgttttcatgctataattacatttgatttttcctgatatttctgtagactttgttcatggttccatgactaggcagaatagtttcatagaatatcctcagcctagttaatgcctagttcccccaaccctgtcaaaaagacagagactaattataggaatttttcaaccaaaaaagacaaaatgttttttttttaatttacacaacctttagagattttatttcaacaaaaacatgatcaacaaaatgaagaaaaaagaattttatacttgaaggaaacagagcaaggggcccttctgagctctgaaaccccttgagacacaaactggcacttattaatattttttttcttttgagatactgtttcactctgtcacccaagctggagtacagtggcatgatcttggctcactgcaacctccacctccaaggttcaagggattctcgtgcctcagcatcctgagtagctgggattacaagcacacaccactgtgtccagctaatttttgtatttttagtagaaacagggttttaccatgttggccaggctggtctcgaactcctgacctcaaatggtctgcctgcctcagcctcccaaagtgctgggaacacaggcgtgagccactgtgcctggcctacttattaataattttattcttgtttttaatgaatatttgttggctaaaacagaactgatttgatctttgggtatatagtcccaaatggttctgtcactggaagacggatgaatccggcagcacagtcaagctaccggatgacataataccaggaggccatcagatatgtgaagagtcagcatccattactgagaaatgagaatctctgcctatgaaatccacataaatctctaaatgagaagatgcaaacaattagaaaattatgttgaaatccagcgtattattatcttgtctctatcttcactcacatcgtcttagtttagctctcataccccagtgaccccttagcactcaatgcctttttcttagcatttaattcctattctccaggttttcatggctatagaacacaggatgacatctcaaatatgtttacactttagggcttccccctcaaaacatgatgaaaggaaatataggaataaaatggattatcatagtggaggtgtggaatgaagagtggtttctccttgcaaatggaatcagttcagggtgtagatgaatactgcttggcctgacctgggttgcaggttcttatcataaattgagtaaagtttgcattacatttccccaagttgagatacatttcttgaaatgccaactattagaacgttgaaaacacaatttcggctctctcttctcaaatccctgcagtctggctcctggcccagccactcaacccaaatcaccctctgaggtcttccaaaataacttcccagccaaaaccaatagtatccttctagacttgtcagctttattcaaaaccttattcttctccctgctaacacccatctggtctcaaattttaagggtttgtcccttccaggttttttctttctcttatcttcggagttgcacctatgagcagttcttctttagacatcattccaactttgataattttaaacacaatgtttgagcatgaattacttccctattcccatccctatccccaaatgctcctttctttttagtcttatatctcttgatgctaggccttcagctgcagttaattatggtaaagtaacaactctggtcttgattttcgacttcttgctgctgtttaatatcttggtttctgtaaacaaaaattgagtcattttaactctttactgaccccatcacttcccaaagcatatattttttacctggattacacttagagaactaatgttcttttagctattctcacccctcccttcttcaaccttagtcaaatgcaggttagaccaaactctacacatgtttggatagggagattcctcttaccttgttgataaaaatgcaattaaaaattaagtgatggtcgcaccatcctctgccagctcttcctgtttcacctccagataacattctctactcaagtcctataaatacccagttgacctatttttctctatcttttaattcttattatttgtcattattcttaaaaagacatcctttctttttagtaagtttgctccattcttcagcaccccatccaaatacctttctccagaatactttccatagattatatgtaaactgggtcatatgaactagctgatttccttcctccttctctcctcaacatacagctttatgtgaaaatgtatgctgagtcatcgttacgtatgtctgaattcccaggtgtgtctgtgcatttggtatgtctgaaccttctggaaagtcacctttttggaaaactgagaaatcttttgaatcttcagtaaaaacccatgcagtcttctccctcttccaccacagcaatatgtgcttaaaatatttttaataataagcgcttactgaaattttacctcattatgtgtacattttctttacaggatccccccatggctgtaacccttggactccgcatggaagaaatgatttttaatcttgctgatactcatttgttttttaatgatttagaggtaagaattttacaaggtacaaatagttgtatccaacaccagacctcctagatggtttctgtatctgtagataacctcgagaaaacagaaagataaaaattcagctcaagagaagcataaagttcttgtcaagaggaaaaattgtatatacaaatataagcagaaagttaatttgtgttcatgaataatacatatgtagaacaaaaatgtctctgaagaggagattgttggctttgctgtccttactgtccacagctgtacatgactcttggttcttttagtgtttcagcattagctcttccagattattaacccggaacctatctaggttaatcactaaacgatattcacagacactattcatggtgatagcgtggatcaattaattttgtggataatctgaaacttaactttagctaacattatcattccccgtttctttcccttctggataaaacagtgctagaactatttaagcacatttgatgcatttaaatgcatttattttctcattttaaaaattatgtttctattttcaacctcaaatggcaagacagtgtctaaatggcaaataagaggaatagaaaataaaaatcaacagcaaaatgaaacgacccaaggaaaaatagcctcagagtagttgaatgtgtactgcaataaagtatgccatctgtgtgctaatgacctaggttattagacattcagataatagaattcgaaataactaaatagttcaggttcttggcagaagtacttctcagtcaagcataactatgctgaaatagagatttctctttcattttttttcagatggcatgtttaaatttattttttatctcatagtttcataattaaagtttttatctatagtcatattatgaattgagagggagattactactactgaacccctaccttgttctagatattttatgttggactttccattaattacttaacaatgtattgaagtaaatgtgtttatcacattatttatgcaagggagaaactgtatggtccacaacatacaaatcacatctaaagttacctgctatctaaatggcagaactggaatttgaagcagatttgcgtggctccagagcacatacttctgacccatcctttgctgaaaattttacaagtgtgtgagaaagaaaagccaagttattgagttacttaaataacttttttttttaatgctctaagttctggagtacatgcgcagaacgtgcaggtttgttacatagatatacacatgctgtggtggtttgctgcacccatcaacccgtcatctacattaggtatttctcctaatgttatccctctcctagccctccaccccatcacaggccccagtgtgtggtgttcccctccctgtgtccatgtgttctcattattcaactcccacttatgagtgagaacatgcattgtttggttttctgttcttatgttagtttgctgagaatgacgatttccagcttcatacatgtccctgcaaaggacatgaactcatcattttttatggctgcatagtattctatggtgtatatgtgccacattttctttatccagtctatcattgatgggcatttgggttggttccaagtctttgctattgaacattgctgcagtaaaaatacatgtgcatgtgtctttatagtagaatgattaataatcctttgggtatatacccagtagtggaattgctgggtcaaatggtatttctagttctagatgcttgaggagttgccacactgtcttccacaatggttgaactaatttacactcccaccaacacgccagttagaaagtttttttttttaaatgacaaggtcttaaatccagtttgagttatatttgagacatgagagtaaaaatgagtggttcagtacattccataaatgtgtgtgctgaaatctgtgtaatagcattttgctgttgttgttgttgtttttctctatggtgagtcatgtttttactgaataatacaattactgtctgtttaaagcggcattcttttttttctacagtggacagtatgaaagctcatattactaggcaagctttcagttttagataacaaataataatcacctctctaaaacctgatttaaattgatcctgctacttatgaatacagaatgattttatatatatatatctggtaataacaaacaccttaacctttccacaacttgaattttttacatacagcgataacttttggggttagtttttctgttgatcagtatggatgagttgcattgtaaagaggatcttaatattattagtcttttcaaaggtataaaaagaaaaaagaaattttatgtggtagtatccaagtaaaagtgcttatttcatttggttaattattttttactggaaataagtggaataaatgaacttttagcttttgtagtggcacattagaagccagatggaataaatttgctacccaacttttaaaagaacactgcagtgagaaggtgaaagtagcatataatgaatttgtaactaactagcaagtttgaaatgaatattggtatatcaaaaacatgttgtagtcatgtatggcacttaaagacatttattttagttcaggtactatgttatatgtatttcaaaaatacctggcaggatctttggtagaagtagttcatgagaaaatactggtattgactttctcttctctgagtatatagctttttagttgtgttttagaatgtgataagaatttcatttgactcaaaccctagaacttagctattcaacaatactgtacttaatcaataaaataattgagtttctgcagataaattgaactctcactgtatatgcgtgccacacaaatgcttgcacatccagataaagtgcccaaggaaacataaatgatttttactgacttgagtaaacaagcactaaaaccaatagaatcaaaacagtagctttcaaaagcattcagtacagttacatacatagcgccctatccttctgcatctgactttgaaaggcagttttactttcacacaactttttatttcaataaacctcttacagccaaaaattgtcttgggctccataactggaattaaatgatttctactcttgttgacaaaaagaattcctcaagagagtaactctaacattagcttattatgttgtcattgttgatgttgaatatttaagctctcagggtgcttttcttaaaaaaaaaaaaaaaaccgaaccatctaatttgacactccattctttcagtagatactgcaacatatatagtagcagctgttcctcacataattccttaaaagatacaaatttaaatcctcctaatagacacttcactgcatcctgcccaactgcagagaaggcagatgcccaaactaccctcccccaacagaagagaatcctgtgtgtttaaataaagtgctatttcggttagaataacctccagaaaagaagtctcaggaagtttgagatggcaaaggtgagaaaagactgctgtgaattataactgataaatatcagtgaccactttttggtcactttgtgagacaaaattaaagtatttaaagttagttctagaatattttcaaatgtttgagttttattaaaatttgttaaagccaaaacattacagtgtttttagtatttcactttttggtcaaaatgattcttacaaagtttctcagagtgaaaaataattgaaggacaattatttagctttttctaaaaatgtaaataatggaagttcaggtttctttcactggaccgtaatcctcaggcaaccgtccagagtcaacaggcatagttaactaggcataaatttaaagaaaagttaaatttggaaaaacctcaaatataacctcaattatggagcttatggtccaataagtgtgatatactgttgtcctcaggagtaaaagataagtttttgggcaacctatgtacaaattaatcatgcgagttaatatttaatgtcagaggaatttctctaaaaattacaatgagaggatattaaatagagaaataacttctgattaccagggaaatttacaaggattcctccacttttttgaatgtcattaatttattataaaagagaaacacagaagttatttacatgtggaaagatttcagagcttcaatggaatgggcaacttcatgtgacaccatttcaatagtgatttatttcagtctacggactttccaagtatgtcaccatctctaaataagaaataatctcatcatttagaactactttggtgcctccatattctgggagaaaaactttatttccaacccttatgctaatgggtaaaatctctccactcttttccttaaagcccagtccagcagttactatcattgcttgcaatgcctttactggatgtttttcaggaagcataatgcctcctttggttacagtttggactgcactcctttcagccaatacttggtcaaagaggggaagacattttctaaatacctgtccggccaggccattccttgggcagtctctgcactcttctctctcccttccttccactcttgacataaatatcttcttactctcctcaccatttagaatgcttttattttatatcatcgtaaccttgtgttagaaaatttaaatgattgtggtaatcctgtaaagaattagcatttgtctagacttctcccatttatttaagaaagaattgtggagtgattattctatgctagattgtgccgtaagtggggatgtaacctggtgaaccaaatgccgcactccagggcacttccagtcagattaaatttggggacccatacgtgtaggtggagatttattcaaaacttactaaccctgtaatttctgtaaattatatgaacttttaacttacctggattttcaagttacatactcagaaggtactgattgggctagtcactgagttttgtatattaaaggaagcctgagtcaactatacctttaaactggaaccggattaaacactttctgtgtggaaatctggagactaggtatgaaatatttgactgccagatactttcaagcccaataatgaatataaatagtagctttcaaaaaacctttgtttagtatggtgtattttcaaatttaatgattttttttaaaaattaattttgaatgctaagcactatgtatatagtcagaagctgtaagtatgagtaaaatactttgagggggactaaggaggcccaagatgctaaaccatacttactctgaggttaatatgatgtagttatttattataaataatttatttttatttattatatatttattattctgagaactaggcattttaaatgccctcaccaaatttcacctttagatcttgtaatattgcgctggaactttgcagattatttgaagatggactaagactcagaacagcaaagaaggatcaaatccttatccacataagttaccatttgttctgagcacacagtaacttacaatcaggtcaactgtggccgggcgtggtggctcacacctgtaatcccagcactttgggaggccgaaacaggcggatcacaaggtcaggagatcgagaccatcctggctaacacagtaaaacatcatctctagtaaaaatacaaaaaacacaaaaaaattagccaggcgtggtggcgggcgcctgtagtcccagctactcgggaggctgaggcaggagaatggcgtgaacccaggaggcagagcttgcagtgagccgagatcgcgccactgcactccaacctgggcgactaagtgagattccatataaaaaaaaaaaaaatcaggtcaattgtgtgggtatacttacataaagtttattttgcttggaattttgtagcacatttgagatgataaatcgtcattatttcccttttacttaaaagtagtctttttgatttgaaaatttaaaaacttgacaaaaagaaaatgtcaatatctatgatcattaccctttttctgtgtcaaaagactaggaccttgaaataacaccagcatggttctttcatggttctggtccagcaggaagtcatggaaattccacaggacaccataatctgaaggcaagcccctctgttgccactcaacaaatgataaataatagtaaccatctgaggtgttcagactaagttttgtgtatcttcagattagccatgcttaagtatgcacaacagaaatggcctggaagggaactcaaatgcttatgaagattcaagagaatttcttatcaataattttaagattaaaatttttaaattgctctaaagaaactctcatgccgtatattttctcacccctcctttctatggaactcttctggatttaaattttgtggggtagaaatggtgccatttcccaacagaagaaaacaagagtgaggcaatgagatggacgggcacatgctgccatcttctggtggtagtctgatcagatgagaagatgggaaaaccttcaagcatggtaacaagcatcatttagaaaacaaataacttttccaatgtttttaacaggagtgtgatcaagttcatatagatgatgtttcctctgatgataatgggcaggacttaaggtaagctatgcctttcagtatgctgtttcctacagaaattcggctgtggagttttgcacatgtaattatttctttttaatgtattttgggtgttacagtacctacagttttgcaactgatggcttccatgcagctgcaagtagtgcaaacctttgtttgccaacaggtgtaagaggaggggttgactggatgaggaagttggcttttcgttacagaagagtaaaagaattatataacacctacaagaacaacgttggaggtatgtgtggctttttcaatctaacaaaggtactctgggtataggtagaattcaatctgtagtttcatgttaaagatgattctgctgtaaaagaaacaagcaaaagctcatattaaaccacattgagccagaagtttcttagaattggtgggaaaaaatttaactacaaaataaaacatgcaccaccgtcacataaattgggaattctttgtgtaaccttgtgcccagatgtttggtcaagatttataccagggcatttagttgaccctttatcagctggtgcttggagcattatcagtgaaaatcagacagaggaactaaccgaggcaacagaaagaagtcctttctcaagtagagagcctcaatcattgaaggacggtggttagcaagcattatctttctggaatcccatcaaaaataaatataaacagttattaatgtcatttcaaagactttcaacaggtttattttatcttttaaaaaaatcatgttttatgtcagtgtatcattctcatgcatagtgtcaactatgattatcatagtttttaaaagttcaaataatctgtaggtaatatcaaaaccattttataccccttcatatattttacaggtgatatcaaaacctttgctatcaaaactgtgatcctggatgtgcttctatatataaaataaaccttggtgatatcttattagaatcaattttaggtaaacaagattcctaaaatttgctattaggtcacatcagattaatctcccaggattttgtttctttatgtgcttctctttttttctaaaaaggaagagtaattgaggaacataaactagaatccaatatactaaatatacatgtgcatactatacatacatatattcacatatatatcatagaatacaatatagtaaataaaatgttcatatatagtatataatcacatatatccatatacatcatgagggtggggctatgtgtctggtcaccttgtatccccagtgcctagcagtgaaacacataagagatgctcaacagatacataataactgtaagagaacaaaaaatgtagagccaaagaggataataatgttagcaacaaaagggagcttagatatggaagacattcatttccaaaaagaaattgacccctgcaagctgccgacttgcatggggtgctggggagaatctctgatttcctgatttgctctagtgctctgtacatgatgcttttgtaaaatcaaataagtagattttctagttaaaatcaatggtagataatatgttttaactttaaaataaatatccccttgggaaatctgagttaatttttatcattcagatgtcatcatttccttcttgcggtgtattaataaaattattttaagttaacaagaagactaagaagtaatcttgtatcttagaaactttaaacaaccacgagtctactttctgtcatacgtgagcatctctctattataatgaggacaaatgagacttgctagaaactgtaaattttgattgaaaaaaccatacaagtgtgtatgtgctcccgatattctaaaacacaatttcaataagaaaccttcctttttaatggatttacatgatcttgttttatcataaaagcaagaactggatcttttaaaataaaaaattagcacatctatcttttttcttttaacatcaatattataattcatgattgtaactgtcctacacttcatgattttaaacaaaattattaattttttacatagcaaagtatttatagtagcattagcagtagtattagaaacattttaacagctacatatgatgatgccattgttactgtttgaaagtggaaatgattgtgattattaagaagctaaacgttttcagacagtcaaaacatttgggatttgtgtccctaggttggtgaagcccaactattcctccagacctcaaatatcacagtcccttggaataagatccaggcaggggcatgttcaaagctctttagatgattgagatccactgcgctagattgtaggatcagtgagggcagagactggggccacttttcagtggcactacctgagacctagaagttcagtaaatattcattgacggaatcaatggatattcaattcagaaacgaaggatcaatatggacataaatctgtatctctctgcatttctgatatatactgaaataaaaatgatctattccagacagaagggaatctaaactagatattctgagacaatattcatctctcgactctgccttggttttttggtgttgcaggactccttggccctgccaagagggatgcctggctacagttaagggcagagattgaaggtctgacagattcctggctaacaaatgcacttaagtctttatcaattattagcactaggtaagtggaattgttacctttctatgtgtatcgtattgaagatttagttctaattgtacaacatagaaaaaatgttttaattcctagactgttttagactttaagggacctaacaattctgagttcagtgattttttcaatttcagtgcatgtgcatgagagagagagagagagagtgtgtgtgtgagtgtgtgtgtgtgtgtgtgtgtgtgtgtgtgtgttgggatgagagaaggttgctgcttctcagaataaacaggagaccctatctcaccacccatctcttctcatagcaactgataatccccacagaatggttacatgacttctaatgaaaaaagaaagagaactcagttggtggcaaagtacaagggggtgatggcaaatatgaaacagtactgttgatacaaaaaggcatgttttaggtgttaggccataatggataatacacataatgtaagcacatcagaaaagcaggagaaagctcttgaaaaaattctaaccattttgttgtttgaactaaacagtgaaattctgacttgtgtcagtatacagagcagaattaaattgatttgtaaccagttgtacaaccaggtgccaaatatgctaattcataaagtgatgtaagtaagtctggaacgtgatagctggtaaggtagagttgaatctctgtactgtccgcttccattattttatatctgagttaggtgaataaataaaagcatgcttaataagtgggagctaaatgatgagaactcatggacacatatagggaaacaacagacactggggcctacttgagggtgaagggtaggaggagggagagaatcagaaagagtaactgttaggtactagacttagtacctgagtgataaaataatctgtatagcaaaaccccgtgacctatatagcaaacatgcacatgtatccctgcacctaaaataaaagtcaaaaaaaaagaatctgtcagcatttccattaagaaaagattaatgggtcaggcgcagtggctcatgcctataatcccagcactttgggaggccgaggcaggtggatcacgaggtcaggagattgagaccaccctagccaacatggtgaaaccccatctctactaaaaatacaaaaaaaaaaaaacaaaattagctggtcgtggtggtgcacacctgtagtcccagctactcgggaggctgaggcaggagagttgcttgaacctgggaggcggaggttacactgagctgagatcatgctactgtactccagcctggagacagagcaagactctgtctcaaaaaaaaaaaaaagtaaagaaaagattaatgagaacgtgcagtatttgcttttttgttcctgtgttagtttgttaaggataatggcctccagctccatccatgtcctggcaaaggacatgatctcattcttctttatggctgcatagtattccatggtgtacatgtaccacattttctttatccagtctatcattgatgcacagttaggttgactccatgtctttgctattgtgaatagtgctgcagtgaacatgtgtgtgcatgtgtctttattataaaatgatttatatgcctttgggtatatacccagtactgggattgctgggttaaatagtagttttgtctttagatctttgaggattcgccacactgccttccacaatggctgaactaatttacactcccaccaaatgccgcatgttctcacttacaagtgagagctaaatgatgagaacatgtggacacatagaggggaacaacacacactggggccttccagagggtggagaatagaaggagggagaggatcagcaaaaagaaaaatgggtattaggcttaatacctggatgatgaaataatctgtacaacaaccccccatgacacaagttaacctatgtaaccaacctgcccttgtacccctgaacttaaaataaaagttggaaaagaaaagatcaatgaataagatgacaaattgttagttttaaaaaaagaagaaaaaagcatgcttaataaatgtatggggaacacagcattcagagaaagatttatttggactaattgaatcagaatgcagaaaagcctgacagggcaaaaaaaaaaaaagttaatattaagaacatgaaagtaaatgaatagttgtcacatcctgcacagcttctgactcgtggcacaggcagagctgcatggatagcagaaagtccaaatggagagcggaggggaagtagcttcctgtgtctgactggggaagccatggcagcttagtttgtcagttctagggaccatattccagtgggggctgtggcagggttgaagcagcctgtgtcaagtgtggcagtcacgggtttggactgtgggaaccacctaaaagaaacagatggaaggaggagccagagagcttagtcagaagagtcatgtgcagaaccaagattaccaatttcacctgtttcagacgttaggctgaaaatcaattagatttctccctgtgtctggaaaggaaagaactgggaccaatgagtaggaatcctgcaagggagattctggctctatataaggaagaaatgtctaaaccagttgaaatgggacttgctatctcatgtcctctgtaattggtgacaagagagaccaaatgtcagggacgctggagcacgaggacccctgagccactgacatgatgtgtaaaatgtagtggagtgtgcatttctctgagaaggaggttgatagctttcattatttctaaaagatgtcttctatataagttaaaaaagatgattacattattagattatatatactatataatatatacagattaattattaaacagatggctttaaggcaactaaaggaatatgtgatattccagaaataaataaagatataacaaggaagggaggagaaaaagaaatcaacatacattgagtagttacatgctatggaatttgctgagttatccacatatattaccatattgatacaattgcagttttgccattacttttaatggcaaaaaacaaaattgcttttgcaccaacccaatagttttcacaaaatgcctggaatgtcagaaaaacagatgctttgttgaattatgtagtagaacccctagcaaaattaacttaccctctgaccctaattattctgtgaatatagtctcagaataacgaaagatgtaagaatatattttggtagcattacttgtaatatttagaaatcaaaagccacttaagcaactcaatttaactctaaaaaattctggtgcatcccacaccacacctattccaaaattgaccacatacttggaagtaaagctctcctcagcaaatgtaaaagaacagaaattataacagactatctctcagaccacagtgcaatcaaactagaactcaggattaagaatctcactcgaaaccgctcaactacatggaaactgaacaacctgctcctgaatgactactgggtacataacgaaatgaaggcagaaataaagatgttctttgaaaccaacgagaacaaagacacaacataccagaatctctgggacgcattcaaagcagtgtgtagagggaaatttatagcactaaatgcccacaagagaaagcaggaaagatccaaaattgacaccctaacatcgcaattaaaagaactagaaaagcaagagcaaacacattcaaaagctagcagaaggcaagaaataactaaaatcagagcagaactgaaggaaatagagacacaaaaaaaccttcaaaaaattaatgaatccaggagctggttttttgaaaggatcaacaaaattgatagaccgctagcaagactaataaagaaaaaaagagagaagaatcaaatagacgcaataaaaaatgataaaggggatatcaccaccaatcccacagaaatacaaactaccatcagaatactacaaacacctctacgcaaataaactagaaaatctagaagaaatggatacattcctcgacacatacactctcccaagactaaaccaggaagaagttgaatctctgaatagaccaataacaggatctgaaattgtggcaataatccatagcttaccaaccaaaaagagtccaggaccagatggattcacagccgaattctaccagaggtacaaggaggaactggtaccattccttctgaaactattccaatcaatagaaaaagagggaatcctctctaactcattttatgaggccagcatcattctgataccaaagccaggcagagacacaacaaaaaaagagaattttagaccaatatccttgatgaacattgatgcaaaaatcctcaataaaatactggcaaaacgaatccagcagcacatcaaaaagcttatccaccatgatcaaatgggcttcatccctgggatgcaaggctggttcaatatatgcaaatcaataaatgtaatccagcatataaacagagccaaagacaaaaaccacatgattatctcaatagatgcagaaaaagcctttgacaaaattcaacaacccttcatgctaaaaactctcaattgatgggacgtatttcaaaataataagagctatctatgacaaacccacagccaatatcatactgaatgggcaaaaactggaagcgttccctttgaaaactggcacaagacagggatgccctctctcaccactcctattcaacatagtgttggaagttctggccagggcaattaggcaggagaaagaaagaaagggtattcaattaggaaaagaggaagtcaaattgtccctctttgcagacgacatgattgtatatctagaaaaccccattgtctcagcccaaaatctccttaagctgataagcaacttcagcaaagtctcaggatacaaaatcaatgtacaaaaatcacaagcattcttatacaccaacaacagacaaacagagagccaaatcatgagtgaactcccattcacaattgcttcaaagagaataaaatacctaggaatccaacttacaagggatgtgaaggacctcttcaaggagaactacaaaacactgctcaaggaaataaaagaggatacaaacaaatggaagaacattccatgctcatgggtaggaagaatcaatatcgtgaaaatggccatactgcccaaggtaatttacagattcaatgccatccccatcaagctaccaatgcctttcttcacagaattggaaaaaactactttaaagttcatatggaaccaaaaaagagcccgcatcgccaagtcaatcctaagccaaaagaacaaagctggaggcatcacactacctgacttcaaactatactacaaggctacagtaaccaaaacagcgatatagatcaaaggaacagaacagcgccctcagaaataacaccgcctatctacaactatctgatctttgacaaacctgagaaaaacaagcaatggggaaaggattccctatttaataaatggtgctgggacaactggctagccaaatgtagaaagctgaaactggatcccttccttacaccttatacaaaaatcaattcaagatggattaaagacttaaatgttagacctaaaactataaaaaccctagaagaaaacctaggcattaccattcaggacataggcatgggcaaggacttcatgtctaaaacaccaaaagcaatggcaacaaaagacaaaattgacaaatgggatctaattaaactaaagagcttctgcacagcaaaagaaactaccatcagagtgaacaggcaactgacaaaatgggagaaaattttcgcaacctactcatctgacaaagggctgatatccagaatctacaatgaactcaaacaaatttacaagaaaaaaacaaacaaccccatcaaaaagtgggcgaaggacatgaacagacacttctctaaagaagacatttatgcagccaaaaaacacatgaaaaaatgctcatcatcactggccatcagagaaatgcaaatcaaaaccacaatgagataccatctcacaccagttagaatggcaatcattaaaaagtcaggaaacaacaggtgctggagaggatgtggagaaataggaacacttttacactgttagtgggactgtaaactagttcaaccattgtggaagtcagtgtggcgattcctcagggatctagaactggaaataccatttgacccagccatcccattactgggtatatacccaaaggactataaatcatgctgctataaagacacatgcacacgtatgtttattgcggcattattcacaatagcaaagacttggaaccaacccaaatgtccaacaatgatagactggattaagaaaatgtggcacatatacaccatggaatactatgcagccataaaaaatgatgagttcatgtcctttgtagggacatggatgaaattggaaatcatcattctcagtaaactatcgcaagaacaaaaaaccaaacactgcatattctcactcataggtggaaattgaacaatgagatcacatggacacaggaaggggaatatcacactctggggactgttgtggggtggggggaggggggagggatagcattgggagatatacctaatgctagatgacgagttagtgggtgcagcgcaccagcatggcacatgtatgcatatgtaactaacctgcacaatgtgcacatgtaccctaaaacttaaagtataataataaaaaaaaaaaggaaaaaaaaaaattctggtgcatctacatggtagagtagtttttttttttttttaattatactttaagttctagggtacatgtgcacaacgtgcaggcttgttacatatgtatacgtgtgccatgttggtgtgctatttgtttatatctattaactattattcccacctcccaacaaaacaatgtataaaaactactctaccgtgtagagtatatacatgtataaaactactctaccgtgtatgtactctacattgttttgttgggagaggggaataatagttaatagatataaacaagtaggctcatgatataaatacataaagtagaatacaaaattgaatatacagaatgttcccaatgggaaaacaaaatctgtatatgaacagaaacactgaaagtaaatgcatgaaatggagaaagcagttttctccagatggttaaataatagttaatgttttttgttttcttctttatatttgtcttttacaaattttcctgttacaagtatatacagtgtttgtaatttaaaaagaaatgtaactctgaatatcacacagtgcaagtcacaaaagttcatatccaaaccacatctcgaaactttaaaagcctgtgctttatttcattctaccatgttttcattctgtaacattttgtaacatttgcctacgtatgacttttaatagcatattgttttgacgaaatattttgtagaactttaagcatcacagtatagtcatgggtttgtatattcttcttaataatctgccattattggaggagtgtctgtctgctgataggcaatagtaatgaattttgaaagggaccattaattaaggaatttagagaccacagtgacaattttaaatctattagaagttatttagtattagaaaacaaacatgtatatttcctccctattttaggagtaactgcataaatgtcttggtaacgacaactcaactgatcccagcacttgcgaaggttctactctatagtttaggaggtgctttccccattgagaatatttacagtgcaactaaaataggtaaggaaattattttaaactctgtatggaatgtgtccacatctgtgtgtctctgcctagttccctttttcagcaatttcacttagtacatgaaacaaatttggataaagttcaaattacaaagtcccctacaactcattgtacatgtatgaattttttgagaactgttgcctagtgacattaagaactcagtatctgagtatagatatttcattcttgtatttcacagaatattacagcccatgttatctaaataatatgtaatacctctagccatagtatttaatagtttaattagtttggcttataatgttgcatccttgcatatgcagtaatctagatccaagataaaaatacaattttggctttgcaggggattctttgatggattgaacgagtgcccttaattacagaaaaaaattaagagttttatgtatcagaaaacataataggaatattcataacacatgagcgactggaaatctgcatttccaaaaatgtgcagtagcaatcaaagaaccacaaccttgtttatttagatgatatctgcttgtggagccatctttgatttttaagtgcatttctcgcagactccaacttctaataaacattcaaaagcaaaacttcatcaacttctgccttattgaaagacttaaaactgatgtattccactgtctgtcaaggaaatacttgggaaaataaatgggttgtgtaatatgccttgaaggtcaacaaacttaggctctggcacattgacaagagaagttcaaaatcctaaaatcccccaccaaaaataacctgccctgggctttgaggaggtctgatgttggccctggcaaaaggagcctccagtggcagctgtttcaagagtacagttaaggagagccaagcttccgtagagtaaatttcacattgtcataagcactacagttcagagcctggatatcaataccatacatttttaatctaaatgtgtgtatttatgtcagcaggactaatagaaagaaggtaattttgcttttttgatgaatcaaataattttacagcagaataaacatttattttagtaaagacaatattctttctgttggcgaaatagttccttatattctttaatctgaaacaaaatcatttttattttttccattaagctagtattttttggacttctgaaccatcagattaagaaaaaaggcagaagtagccatggctgcatgaacattctgggagtgaaatttagagaataatgccaatgttttaaaaacaaagaatatgtagaaattccacggtgatgttcccgtagtgcaataagaggataccagtgttgtcagtactgtggcccagtcagacagcacccttagcagaagttatttgagcaggctaactctcacccacttgaagaaaggtgttgtttatcaagataaattcacttcttagtatttcctctttttttagcggtgaaccttcagtttttctctagaacacatttagctgacttgtctatgctacttaaaattgatttacagaattaaacaaaggttaaaacttatagttgcttgtaaagttaccccaatactctgttggtggagaaaaacatactaatattttctctaggtcaaatgcttatccctactgatgtttttggagtgtgaaacacatttgtttaatcatctgtccgaagtaacgccttaccttattcaaatatatgttgatttcataaacccagaggttgtgagccagttcaagtgatgttcaagaggcattaacttaacactttatgaatctttaggttaatgaatgcagtggctggaagaataagcagtgcattgtttttcaggcaaggaaagctgttttgagcgtatagtgtccagatttggcactaacataacttatgttgtgattggagatggccgagatgaggagcatgccgctaaccaggtaacttcactctgaactttatctcatttatcttccaggaaaagaaagttgctttgaacgaataatgcaaaggtttggcagaaaagtagtgtatgttgtaattggggatggtgtagaagaagaacaggcagcaaaaaaggtaacctgtctcaaacaatgtcggtgtgatacttctaatgcaagcctttttgtttgcatttctgtagtttggcccaatggcagcttgacaaatgatttaaatttatagatgcaaagcaataagagcatgagagggtcaatacttacattcaaataaaccatttggagtttagcattttttgcctgaaatttgataggaaatgcagggaaatttagttatatttatttatgtgtttgaaagtatatacatatagtaaatatatatagtaacatatatatttgtgtgggcatgtatacacatgcttatatagatgcatatgtatatattaatacacatacaatgtgcacagatatagatgagccaggggatctgcaagaaagattgggggggagaataaccattgttaagcacatgaaatttagcaagatagagattaatgagctccatacaaaagtcatgtgagtagttcatgagacctgcatgccatatggtcctcgtgagtgcctggaagtaggcatgctccatgataaggaatgctgagggcagatttaatagctgaagaaaaactgctacaagagattttaataagtgctactgaaatgggatgtattgctcagatccttttgcaccaacttgtcttagccgtaatgtgcctcacatctcctagaagagtttcatctgttttggtgagccagtggtacttgattgaagccaagcagctggctttgaacacattcagaagatcccatgggccctaagtattttaataagccttttcatctggcccgtatttctgaagaagaaaccaaggcaattaaaaaccattaaatggttggttcaacatcaaaagaagaatgatatcaggattggcccccaccaccagactgtcaactccaccctaagatctactgctgccttcaccgaatgatagaaaaatatcatctacccaagataaaagtgatagaatttacaaaacaaagccagatgatgaattagtatttttaaaacaatgacatgccagcctatactgttttacatagaaagaatgtcatctgtatcaccctaaggagattttaaatcatcatctaataaagcctttgccatttgcatgtgctggaatgtttacctgttttcaaaaaacagtcaacatagacagttttatagaaaagtttatggctataagagaaagagaaaggaaatgatgtatgtttctagtcgatttaaatcaagcaaaatggagaaaaggaaagcacttccactttagaagttttggtgacgtattggcagccatacttgcttttcgattacatttaagagagtaggctcaggctctgcctgccacagaacacctcagccttcattagcagtgtcaatgacccaagacagcacactagccccaagcacgtcctgtatgaggtcagggctgcagcctcaagaaatacggctttcattgctgactactacttcaaatgcttccagtcacactgaactgaaatatcttctggagtttgtcaatgtcctgagtccatgttgtgatttttcttattggcaccatagttatacacaacaaagagcagccaatgattagtaatacagccccactcctgctaaatttacatgtcttttagcctatgcaaattaaattcacgtatatttaggtaaggactaatataattcagttttccaaaccagtaacacaatatgcttttgacaattaaaacttagccgagtctacctgttttccacagcattaatgtttagattcggtttaccagttgttatacctaaaacttcaggagggaaacgcataaccctgaatggaaaacaatctcagatagaatgctatttccaaatggttgtttatgtaggtacagttttggtgatggtgaaggcttattattctattttaaaacagaggtgattggtttctctagtacccagaatgcatgcttttctttggatgtagcggtaatcgcatgtgcagaaatgttaagtactgttttgttgttacaaatagatctgagctgaatgaaaaaatgttacttctatgtgaatttcagtgacttaatttgacctagacatattttgtctgagaaatttaagaagaaatatcaaagtttaggaataggaatgtctagctgcgctcttccatagaaacatgtaatattaatacaagctgtatatgtagtttgaaattttctagaagctacattttacaaaattaaagagaaacaggtagaattaattttaataacatgtcaacccagtatacccaaaataattgtcttgatatctaattaatgtaaaaattattgaagtcatgtttacattcttttttgtttgtttgttttgtcctaagtctttgaaattcattgtgtattttaaactggaccaaccacatttcaagtgctcaatagccacatgtggctggtggctaccatagtgtgcagcaaggaattatatagcaataaggattattatgctacttagttcttaaataaaatatcttttgcatgttttatatctatatactcatttcccttatatacatgaatattttatacgtacatattctagaagatgtcatcagcccaaacttaacaaatcatgcttttgttttgtcagttcctgtaatgcagtgttttttctttttcattatcatcgccagtggggactctttagacagtttttttctaattactccctttcatgaaattttaatgtcacatatgtactgtatactattatagtatatcttttgaaggccacaaaccactgtaatgtctaagattttctgccccacaagagctcatttgtgtccctttggagatatcttccccattgaaaatgtatgctagagtactttctctcaattctacgccagtgaaataaatcaggtcgactctactggcttaagttattggttgtcccagacaagtaagaaagttataatccccaccttttttttctactttatgtagtcctgcttcaaattatttttatacagaaaggaattcaccctcttttcttcctagattaacaaaattaagaagtttgtcattttgtttcattttatttttaattgagaggcccgtaggagtgatgcactgattaaaacttgctttccattagccaggtaggattttgatgcccacttgctttcccagatgttgatgttggtagcgatacttccagaataaatattaaactatctaagaaatgttaatatttgctattattctcattaaaatccagttcagatctatttctctgttactttgacagtgccatattggtgctgccaagccttcacatatgatcatcccgcctttaaactctctcccctgaacttgggtggaccacacatccctgtgtgtgctgctgcttcattgagacaatggttgtgatctctctccatgcctcattccttccccttctctctcccatccctccttctcctaaccacacagcacaacatgcccttctggaggatatccagtcactcagacctcctggctctccaccaagcactggaattagagtatttgtaactgtgttctttagccggagatccattttttatatttcaagtacactgaatttttatgtgtgattcaatgcctctggctctacacatataaattgtcttaatggatgaaatcatatttggaataaaaattccagaatgaagaattcagattgctgaatggagttaaactttagtgctacagaaaagaaactctatggtcttatatttacaacactttaatgggttttttaaaaatctgtggaggttgctggtacacaccaaatgagtccaaactggaatgagcagctttagcaaagaactcttaccctggcaaagcagcaacacacatgctccgtctgacaaggtggtcaacaacattcctcaaaatgggagatcttctcagccctgaggtttgaatctgactttagcctacctaacccagaaaatctgaattggaatgcactcagactgtataaggacagtcctatttagacatgtaatttgtgtaaattattgatgaaaataatttactgtgactttattagcagctgactttcaaagtggatgcaatttttctttcttttgttggggaggggaatgggaggggaaatgggaatataatattgtctcttttttaagtttggcaaacagaatgttcatactgatgtgttgtgccttaaagacaagacagcatttgtgtgttacaatgtaactttggttaaaatctctgtagataatgaaaaaaaacaaaaaaaaaaacctttgtgatgattcttaacatgaccaaatttaaaagtcaagctctcagagcttaattaccgcatcagcaagaaactgagtattttttgcaataagaaaacaacaataataaaggaaagcttgtgtttcatttgggttcttaataattccaataattgtatgaggcaactatttgcgcatccaaccatgagtggaaggtttgggaaagactgtgggacctttacttagaaagtgaaatgtatgtagaagtctcaagtaccccttctacagttttactggagaaaactaagagccatattcatgacaacttgcacagttttgaggttgagacttttgatatgtgtaagttgcatagaggaggatattatcatgcaaatcatgagcaattatcacataaacttttttagaatgtgccatgaacatggcataaaattcacattgagtgcacagggcttaaaataaagctaagtatgtttattcccaatgccatggcaaaaatgataatatcatcagaaatggaaggcagttctcccagatggtgtctaatgaaagcaatgagtctatgaaaattttacctagaatatcatcataaattaaattagcaagtgcgctggatcttggcagcgctgctgaaatgacaacagtaaaataatacctggttctccatctgaatacatcaatgcaggttctcctcgtaacagacttgcatatgtttgttagtttctgcctgtattgtcactgcgcaacggatggcattcattacaagaagagcccatcatcgttgtgtttgcatggtttttttccttgtgtgtagcccatgttgggaacacgatacaggttctcctcttatttcctatgacacgatttcccttgtggaaatttaagactttaagaactagagtatttttatggtgtctgcacctgcagttctgtgtttaaaatgtcataatgtggatcctggagtcaggctactagtcagtgcccctagccagaggctggttcatgagttcatcaactgaggcctctgtggcttcaataaagtctacattttgctcacagatcacaacattcactgtggaaatatgatttcatttctttaggctacaaacctgtatttctttactgaatgctaaggccatgtttatattgggtagaaagatattgagatcccaattttgtacaagattgtgatttcattatctaaaccttaaacttaatcctttaaattttgtagcttttggctgcatctgccccaagtactattccaggcaaattaaagttggaatacctttaataatataaaaataatgatagtaaatcttatacttctgttggcccttagcttgaaaatagcagttaaaaaaatttaaatgttgccttgattatcagtacttaattatgttgtgcactaaaaccttaaatatttattactgtgaataaaaacaaattatctttactgtatagctggtttctttaaatgttgatagaattgtggcattacatctaaatttgtaagtcttttcatatcaaacaagcaaggctttttatgctgctaagtctgtgggtgcagaaagaaacaccccttggaagggcaaagagaagccggctggttgcatcaccccgtgcagtttctcacacacatctctttttctgattctgtgttcagaagaggctgccggcataaaacctaaatgcaaggttgacggagaacagcttgtctggcacaacaatggtgcaggcccacgagccagcatcacagcttggccatgggacgttgagtatgcacaaactagaactcttcccttcccaccttaggaatagaaaatcctcttcctttctaatctgaaaaacgaaaactgaacaaacacaaaaccaaccccttggcagttcccacctcctattgacatatggaatattgtgccttattgtaaaccagtttgaaaaatgttctgtaactaaagtgggttttcggctattatgtatacagcttggtatattactacgaaagataaccaccttgtgttgcaccttaaaaatatcaagaccatgtaatttccataacaaaataggtggcctggctatggtattatagcatacaattaggacactatgcccctgcaaaattttgtaaatcaaattcagaggcaaaaacatattttagaatcatacagtttgcacacgacaagtaggtaataactgtctctaaaaatgttttctccttagtccgcaatgagctaagattcagaatagtgtcaacagcatgctcctatatgaagttgttttttttaaagcacatttgtttatgacaagcctacattctcagtgaatatggcatttagtatttcttttgaaaacaaacttaagcatcatgagccaaagttgcactttgactcccaactacggtagcattatggacatctcacaatgtcaagggtttctgttatgtattagtaaagtatagattattggggcctacataatttaaaagaaatgtaaattaatattaaaagcttgtaaaaatatgtatatctttactatttctcaataaatacctttgcaattgttttcatttccttcaccctcccatgttgtagagtttttgtgtgtttatttagcttggttctaaatcatgtgaaatttgtatttaggtttctggtcttcataacttttcttttttatgcctaagttgcataatttccagttaaagcaaaatgagatgtaacacagtaaaacgtcactaattatgactaaaagtgagctgctttactgaaaagatgtcagttgcataattttttatacacaggtagctacattcaagtaagtataatgcataatgtagccacaattcactaatgataaagtaattaaaatatttagacctattagaatatttaaagcaagtatcgtttacgtgaatatgggacatagtttataagtatcttccaagaggataaaaaggtagttttctgtaacagatttaatagctcctttattgtctggtgtatagtatttatttatttgtgaatctgttgttcacatgtaaaccaaatgcaaatttcagataaacctctgctcattcacaacctttaaaaatgttagcctccaaattaatggtatttaagtggagctatgtggcaacattctagtaagccttacaccatccaaaaatcaatactgatttacagg

SEQ ID NO: 12 - DNA, 13711 bp, Homo sapiens
  source 1..13711: mol_type = other DNA
ccgagagccaggccgcctacaggcgtctccagccctaccccatcgccggctacagagcgggagccccgagccccagcgccgcgccgcgcggggagagaagcagctgttcccaacagctggggggcggcggcgcccggcgccccaggtgctgccgcggccaatcgcaggcgtccggcccgcccccctccccagccgggcgtcgacccccggcccgcgggggaagggagcagagaggtggagagctgtcctcggcactcgtcttccagatcccacggcgggagggacagaccagagccgaagctgcgcggagttcctcgcccctggccccgccgccgcgccccccgcccgcttcggcgctcggacgctcttcccgcggaccctccccacaccccacccaaggcacctgattcctaaggaagaaaaatctacctctaaagcacccgcttcttcgcttgagaactaccctctcttttcatttttggcctccccgccgggtgagtggaaaccgcttgcacattctatagaacccgattctaatccagacgatccggagaatttgtacggtgaccggcagggggggcgggaggagagagcgaagtcaagtctccagggccgctggcgacttcggaagccgcgcgcccggcgctctcggccgcccccggccgctcgcccccgccgcgccgccgccctcgcccccgggctcgcccttggcccccggcggccgcgaaagggtgcgggagacgcggagccggaggaggaggcgcagccgctgcccgagccgcagccgcagccggagcccgagccgcggggcgggtgcgaagatgcacacgacccagaaggacacgacgtacaccaagatcttcgtcggggggctgccctaccacaccaccgacgccagcctgcgcaagtacttcgaggtcttcggcgagatcgaggaggcggtggtcatcaccgaccggcagacgggcaagtcccggggctatggatttgtaagttgcacggactgggggtgacggggagggacggagtggcggctgaccccggggatcgggagcttggagtgaggggctcggcgtgacccgtgaggagccccgcgggtagagcggcgctgccccttcgctccggggtgaactgaaactttgctaggggagagggtcggcgccagcctcgcggggttcggagaagacccagcgctgtgcgaggtcgggggccgggcagggcagagcaggggtgaaaggagagacctgtaatgacggcgggatttggggtgcggagggttgcgagggaggggccgcaaccctgaacaattgcattcccgggctggatgccggttgttaagcgcgctgttgctttgtaaaaatgcgtgtgttctggtgttttggctggggcagggaggggacaccccacgaggtctggggctgttgcccttgtgtcatccccccgccttttaactgcaaaggtaggttcttttaaatcatgatcctagggctctttgccgaactgaaacagagcatattaagcttccttcctagagatttaagcgtgcaaattcagctgccgaagagcagggagccccagagtaggacaatagctattgtcgttggggagtgttttcgaggttgggagcgttgggcggatgtaaggctggactttctttaaagcaaacggtgcctctgaggctgagtagggacctggggggcgggggattcgggggaaggaggatctagagttccaagaatttaggcttccaaatctcggctaggccctactaacacccttaagatttcggcggtggggtggggggaggcagggttagagggtttctttcttttttccttcgtctacccgccccccccccccacgcccccgcccgcctgttaaaaatgggagatcaaggaggagggtccagccaaaagaagcaaagaatagaaaaaaagtttgatctcagttttatcatgaatgacttctccattgtgattcttgatttccttcatgggttaatttagaggttatgtatgtatgtctgtgtgtgtctgttgctaaggtcaccatggctgaccgggctgctgccgaaagggcctgcaaggatcccaatcccatcattgatggcagaaaggccaacgtgaacctggcatacttaggagcaaaaccaaggatcatgcaaccaggtgagaaatgtctgtctcccctacccccctctccaagaaccccccatttatgggtgggatgattctctaatttagaagacattgtctctttctgtagatgtgtttagtaaacttgaaccatggcttgttattctaccaaggcattaaacataaatgagagttgtaaatactcctctcaaggcttgaaatgtacctgtgtgcaaaaatggatcttgggggtcgtgcaggaagaagagttagtcatgtctcacctgtttcctatatggcatacatttgtagtctgctctcaattttccaatttttagccacctgtagataattcacatgtggattatataggccaaatttgtcgacttcagctaatatacttccctacttagtggtgtctctccttttagcaccagttgcactgtgctcagagcaggcagtctgacttgaacacagacccaagggaagcattaaggggtaggggcgatgtccaaatggtaaaggttttgtttttttttgagatggagtttcgttcttgttgcccaggctggagtgcaatgacgcgatctaggctcactgcaacctccgcctcctgggttcaagtgattgtcctgcctcagcctcccaagtagctgggattacaggaatgtgtcaccacacccagctaattttgtatttttagtagagacggggtttcactgtgttggtcaggctggtcttgaactcctgacctcaggtgatccacccgccttggcctcctaaagtgctgggattacaggcgtgagccaccgcgcctggcccaaaatggtaaaccttcaacataaaatcacagagtattagcaatcaaagaaaacagaaattgcacctactcaagaaccctatgctacagacaaagaaactgatgcttagagatgagaaatattttacttaagtcatgtagctattagttgccaaaagggagcagttttttgtctatgggaaaacaatacctcttggattatttacaacactgccctttgcattagcacaaatagttgagagttgatttttaagcaaatataatattttctttggtcaggcaactttacttgtaaagtttgattaaatcatatactcttcactcctaagcctttccatctgaaattttgggaagaggttgacacttccatccaaacgggtttaaaaggaaactaggttgtcaagaaaatgagtgtttgtgcaaatattcatgggcgttaaaaaaatttgtctaatacattgtgggtatgtagtatccagaaaaaaaacatttttttttgtgaatttttaatactggaaagtgtaaaacccataatcttccaaagcattgtctgaagcatcccagaacgttccaaattacaaagacttttcatagattctggttcaaaactgtgagaatatttgagatgtaacttcactgtcaaatcctgctgtacttaattttatttcatgaacattttcccaaggtctgattctgtgtctgtcaactaaaatttgcagatttttttaaagttttcaccttttctctgttaaaacctttattttgaatcaatgatagcatttataaaactgactttaaaaaatatacatacatatagtttctttaaagtggcgaaagaatgtttatcttttataagtgaaagttgtgattgcattttacatgtaggaataataaatgcctaaccatgcacaaataaagaatgtggtatattttgttgttaggttttgcctttggtgttcaacaacttcatccagcccttatacaaagacctttcgggtaagttgattaatcaggctttcttaagtttcagtatgactatcatttgttaacgtgcctctttgttatatacagataagatcatgatgtcacagaatggtcctagttatacaattttatatatatttgtgcatttttaatgctgatactataaatatttttagatgagtcaagttatctgaccaagtatgcagcctccaaacgagacaaattttgagcttatgattagcaaaacaaaacaaaaataccaggtatgacttgtgtacaagacgattgtgtcccatcaccattactactgttcgacttgactcaattttatttatctctttctaacctgacagggctcgtatacagttaagctaactgatcagaaggataccatgataagaggtggaatagttctgtttggaattaaactctgtaaccagtagactcagacacacaagctcagatggtgattaacgatgcatggatgttggtgttgttgtttttatttttcacaatagtcaatagcccttcgtgtgctttattttatttattacagtaatgaagtggtgttgatagctaaactgtgtgctttttacttaattgaaaagagctgtagtgaaagtgttttcttttcctcggggaaggggctctttgcgttttgattttggtggtggtgttgaggggtgttgacttttgactttgccatgggaagcaaagggcctttttttttttcttgcttttaagctgtagcctgtgacttccatttaaccacatttttgtagtgagtgctaaattgtgtgatttctctttgtcagaatacagattcattgcttgcatgtcctaagtttctgttttatgggaaattttctgggaatggggttctttcttttgcaatgtgtatccacttttaggccatctaatttaaaggttcaacaactctcatcagattttattaacacaagccagtttttttgagtagcgtcacactaactttcacctttcagatctaactcaggtgaacacctgttcatatttatgttgtctttgggtcctgtgattaccagttttcctgttccaaggtcctcatttctcttcctttcagaggttatatgggtgggagatcacccctcgcctcaatttatcctggacactagattctgttttggctctgttctacctgatcaacatgcctgtaactctaacgcaggatacttaaattttctttttaaatgttcatgttgttgaaagagtgttttatcctcaataataatttctcctctagcttaggacctaaaacatctacctgtgtaacctggaaggctggaatctgtctacatagatagatgtctaccatatgtgtctctaagaatagattttaaaaagaaattaataataacttgacttggaagagtaaagttatatttcccatgaccacctccttacaactctaacatatcggtacctcactgttattgccaactactactatttttttttttttttttttagctgtggaagagtcttctgttcaatatgatcagaaagggccttcctatatccatagctacttagcaaatccccatccaagccaacccaaatactgaaaacaagtggtgggttgatgggttcagctatttctgtgggtaattcatttagacttaactgtttaaggtgggattagggaggagagcatatatctgaaggtgtgatgtgagcttagtggtgggctcacactaataatcttgaaacagttataaaattgaatcaatcaacaggtagttgcatgtgtttattttttcactgtgccagtgggtgatgcaaaatgagggaattgcacattagtttgggaagcaaaagtcatgcaaaaaccagtcaagaattattaatttctaagtgacttggtattgtttcaagtacaaaaggaattcagagtagcaagaaaatagatgaagtggaggagttgaataaggctgtggtgtgcatggcaggtagactggcactagacttgcaccactgggtatgtcttaggcaggtaaaaggaagactggatatgtctggagggtataggatggagtgagccgaagcagaggcatagaggagaagactcagtgcgtggagaaaccagactgtctaaggtgggtcttgagagctgggcataggaatttggactgattgtggaagtccttatagatcagttcttaagaaagaaggtaacacaaggaaaattacaaccaccagtgtaagatgtgtcatggaaatttgaaagcaagtgtgttttgatagcaaccttttgtttttcacctcttctaatattaactgtctttataaaggatggtgattattagtgtgtgcataaattgtttgaacttgagattcttattagaaatagatagcattccaggtggaccaagtgagctttctctttcttttttaagaggttctcaacactgctctaatttcagagtctgtacctaacaaatcacacctggtcatcctaaattctgtttgaatgctacatgttcctattttaggcctggaaagaaacttgagaatttcaaatttgacttgacttacttgaaattctagtaactgaactgttcctttgaatgtggtccttaaatggcccccaggtgactttgggcctctgtagacaaaaccttggcctaaaaggaatttctgatgtagcacagtgggcagatgtagaaaaacttataaacttgctaaacaatattttatagtttatggttgaaagaagcatttccttattgaaaccttggaaaggggaggctacagatctgtgagtagaaatttctgggctagtggccaggtgcggtggctcatgcctgtaatcccagcactttgggaggccgaggtgggtggatcatgaggtcaggaggttgagagggcgagaccatcctggctaacacagtgaaaccccatctctaataaaaatacaaaaaattagctgggcgtggtggtgggcgcctgtagtccagctactcgggaggctgaggcaggagaatggcgtgaacccagaaggcggagcttgcagcgagccgagattgtgccactgcactccagcctgggcgacagagcgagactccatctcaaaaaaaaaaaccaaaaaacaaatttctgggctagtttagatgaggttgaacatacacttaaacattgactcagtacccctttaagtgctcagttttatgctgggtgtggtaacactgtgcttagtttttatagtgtccactaaggagggcaggagggactgtaactaacactaaccaaacatctgccatatggattgtaatgataagggatttacataaattacataatttaattctcacagtaagtgtttgttgaaggcattgttaatatcttttccaaaactaagacacagagaggttaattaacttccccctgatcacaggactgctaagtgttagaggctgagtctagattcagaatcatggtctgcctgacctcagaagctgcagtctttcctctttgtcaagctgtagccccaaaagacaaaactctattctctcataatccagtaagaaagccgataagcaccaagaagggctgtccatgtggcttcagataagagagacgaacatcaggacaatgagtgggattcagaagtcttcgtgaaggaagtagaagttgacctaggttttaaagaatggtggagggtagggaactcttttattgagtgcctactatatttggcattgtagactctctccacagataattttatttaatgcacagcaatccagggttgtgagattattagtccatttcacatatgcaaaataggaggctatgttaagacaacaaagcataaaaaaagaaacatatgagactaaacgataaagcaaatttctgaaacctctacacgtagtgagtgctagagctagggtgagtgggatttggatttcagcaggtagagacagtgtgaggattagcatctgggtgagggatgaatgacatgaataaagggcaagaatctgggaatgtgaagcttgtccaggaaacataaaggagcccagttgacctgggtttgaggaaaggtggaaggaaatactagaaaggtgttttggagccaggggcagtctggatgttttggcctagaatgccaaggcttaagaaatttgcactgagtttggttttttaggggccaaggattactttgagcaaatgaatgagatggagtatttgtcatcagttgctgtgagatagattggaaaaagaaaccgattaggatattgcttcaaaaccagttaggatgttgctttaaaagtcacatagagatgtcagtggaacctgaagagatgggaagaaaaggaaaatgtagtaaattttctttgaaaattaccaaaagtgtcttctttgcacttgtgctttttgtaactgctaggcatgcacgttttatggttaaggaaacgacattggtaaatgtctaataggagggagattcccagatcaccttaatgggtctcagactattaaaaagaactcctaattcaggatttcattccaaagtgccacttgaaccttcagtccaattctgccttccttcctgacccaggatattttaggctctccaaggtctttcgggttctccttttccctctaagccttaaaatgttctaggttttcatttgccttccagacgtctctttaacctttgccaaacttcatgtaggagtctgataatccagtgaactttctttatattctcattgtttgacattgtgaaatcaaaactatttcctaatgcaaagtaaatgcagagctaaagccccctaagcaaccttgcctgtcttttaatgcattcttcacaacactttctcctttttgtaggataaacattttcacttaacaccttaattcagagataccaacctagcacccacagtgttgttcttggaactttgagagtacaaggggttaaggtttcggtgtcagtgagatccgcactggctggcctttgcagctgatagatgttttactttggccttataaagacattttacccgctggaaacccttgtcgcatgacaggatcaaaaatagcccggctgcagcccctgctgtccatcaccacatgtaaactggcataagagtaggctctcttagccgtgtctacacgttgtagacacggcagcctccagggacagtgtttcataaagccacacctgttgttgcgagtgagccagtcacctgacctgcagcaaagttctgagtgtgtgcatgaccacgcgcctggtttctgacaagcagttaagaacatctctgagtgtcctcagaaacacttgatgacgtcgtttctcctggcactgggaaacatctacaagtctggtaaaattgagtgtatttttatcattggggccaaagtccactaccctctgagataaattcatcttttccatggatgttcgttaatgagtgctttcagtaaaactctgaaaaggcaaagcaaaatctatgtttctgaaaaatctacaagtgctgttttctgttttgagttctttgaaattgaaaggttgttacttgtccttgtacttcttacaagttggctttgccagcaaccacgtagtattatagtgtcatgtttttagatataaatatagggggctttaatggagattattttaaagaagaagataaatcagatatttatggtagtgtttcagatgggcagtaataaattgcaagagtgagaatggagatgcatggaatttttgtttaaatacctcttgtgatttatttttttaaccacattaacttttaagtaacgtcattggagtctgtcttgtattatctgtgaatgtataattttaaaagatacttggttttgtttgactttcttgaacttttgcaggataacacatgattatacaattgatatgatttgtacaaaatatcaacgaatagggcagatttcgaacataacactaacgtatttctgctaatttggaggatgaaacaagagttagtagatgtgcaaaaaaagtttccttgttatgtaaaccttttaaatttctattaattttagaaatatttagttgtacttccctgtcctgccttttccctcaacctcatcatttccccaaaaccccttctgtctgtcttgcatagagataaacaagctggctgaccctcaggtgcctgatagtcaaacaggtttggttgaccgttgtgtccttatgaattttatgggggaaaaaaaagagaaagaaagaaaataagtgtgctgcacatctctttgggaatcactgccatgaatgttaatagcatttttgtagcttcccacgacagcctcttgtgacagagtgagctgcactacagttcgtttgagaagtgtgattctgctgtgtttaccacacttgtgaacttttaaactcctttttcttattttgtcgcctgcttccttttttaattattatttttaataagggtgactgaggttgcagagagattaatgtgtgctgagtctgcatagcaatttcaagtagggttgccatgataataaacatacggattttgttaacgtttatgttaatttcgacaaactggtgatcaccccacagaagtgaataattatggactaccttttgagtcctatattcttggctaagaatttttctatttacttaattaaacattcttattttatgaactcattacaaaattttccaaagtcttcatcaattgagcacttgttaaacctctcttacgtgtgtagccctcatgaatcttgatttattttccaaactcttgataaactgtacttagcagctccaaataatcatacatgacaattctagcagggagggtgtcatcatatttttggggtccagttcttctgggggcactctatgactttggctactgcctgagtattctcaatggaccactagaaagtagatattgtggcaaccctatgctcataataaccactaaatttgagtttgttttatctttgaacaacatgctacagttaggaggtgactaccgcctgactttgttttccatttctcaacaggatacctgcccactatgtctatccgcaggcttttgtgcagccgggagtggtcattccacacgtccagccgacagcagctgccgcctccaccaccccttacattgattacactggagctgcatacgcacaatactcagcagctgctgctgctgccgccgccgctgctgcctatgaccagtacccctatgcagcctctccagctgctgcaggatatgttactgctgggggctatggctacgcagtccagcagccaatcaccgcagcggcacctgggacagctgccgccgccgctgcagcagctgctgccgctgcagcatttggccagtaccagcctcagcagctgcagacagaccgaatgcaatagaccagccatctgatcaaagttgaattgttttctctttccctcccaattttccaatttttagtagctaataagagagttaacattgacttaacagctttaaaaaaaaaaacagccatgctattgtgaagcagagttattattttttttatactccaggtagtgttctagatgagaaagaggtaagaatgaggggaatgggcacaattttggaaatcaatcccaaagagcctgagtaaatgaaaggccactacgaaatgacgccaggagtaacaacggaacttcacttttgtaacgggatttttatttttgctctttttatagtatcagggaagcaaactgccttttacaagttagaaaatgctgtttgaatctagttgaaccagggaatacagagcgagcaatatgtagcttgaattacatttaaaagcagattttttacaaacaaaatggcgaaagcactgattgtcatttttagcagtcacttaaggcctatagaacttttttcaagtcggaaggtcctgttcttactatctcaaaaatgggcatcgaacaatcaatctaggagcgtggcagtgggtaaaatggtggacaggcaccaaagctattttctcatctgtcctgtggatgagtggaactgtggaacaagtgatgtggaattaatgggtgcaacagctgtacagacaatcaataacacacacagttctggaaagaacacatcacttgtgcttgtttgatgagcttgtcacattctaatccctctccccatcctgtttcaattttgggaaacttgtatctgctggtgtcagcaattctgattctgaaataggatcaggcttttactacaacagccttctctttctatttattgtgtcgactcgtggcttatgaataaaggcaggcaaagtttgcagaactgtcttaaggacatttattttttttccctttttaagtaattttacactttagtatctatttcagagtcatatttaaaactatttattagtgactacagtacatgagacaacaaggttactgagattacaattcttcaaaggttaatgataatgtggtttatactgtgccttaatagtaatgctatttaagatatttatttttaagttttactatgctgcactctaaagaaaggaactttagatgtgacactgtaaaattatgtattcatctcatggcataaattatttagtaagtctagatgtagcgtattaaatattaacctattcaactaaagatgttgacttggatttatttaaattcatatgtgcactgtataagagagtactcttacattaacacattttagtttagtttttagatttttttttaaacgatatattgctagtttcatgcttcctcctctgattttgcctgtgtagatttcatttattggtaattcattagtttaacactattatgtagtttttaaatgctgctttacatttttcttctgaaactgcaatacttgcaatttttattcttaaactaaattgaatactattttacactgtattggatttttatacttgaacaatttcatacaagggaagacaggttagcatttttatggactttctccattatcactggatttactttaagtattcccatactagacagtgttatgtaatgtagacatgactctcctgtgcaaattatttattcgttgtgtatattgctttataacatttcagatcttctaatctattcacttgtattaaatataatttttaaaaacttctgttgcattggctgtttctaattctgtagtaaaggtcaatgatttctcttttgaacttgaatcaatagccatgattgatacgtaacattttcagtgagcataatactctcttggatgtcctggttttgtgcctttaaagtaagtttcactaactgaatatctaccttatacttagcaaatgatataggtcatcccctgtcccaccaggggagccctaaaggacttcgtatcactgaaaactggttagctgaaagatggcttggaaggcgtgggccagtagtagagaggactagtcagaagtgggaccagcccagtggtgctcgggtcagctaaactcaggtttgagctggactccccacttttcagcaatgttagtgaagccaattgctcttcgtaattcacctttttttttttttttttttttgagacagagtcttgctctgtcacccagtctggagtgcagtggcacgacctcggctcactgcaagctccgcctcccaggttcacgccattctcctgccttagcctcccgcgtagctgggactgcaggcacccgccacctcgcctggctaattttttgtatttttagtagagac

SEQ ID NO: 13 - DNA, 22 bp, synthetic construct
  source 1..22: mol_type = other DNA
atcgctgctgagtgaaccacag

SEQ ID NO: 14 - DNA, 22 bp, synthetic construct
  source 1..22: mol_type = other DNA
ctactctctgcctcacaggtca

SEQ ID NO: 15 - DNA, 22 bp, synthetic construct
  source 1..22: mol_type = other DNA
aacctcgatggcttggtggcaa

SEQ ID NO: 16 - DNA, 22 bp, synthetic construct
  source 1..22: mol_type = other DNA
tcaggatgtggcactcatagcc

SEQ ID NO: 17 - DNA, 20 bp, synthetic construct
  source 1..20: mol_type = other DNA
aagcatggcctgtacaacct

SEQ ID NO: 18 - DNA, 19 bp, synthetic construct
  source 1..19: mol_type = other DNA
gggttcacacaccagcact

SEQ ID NO: 19 - DNA, 19 bp, synthetic construct
  source 1..19: mol_type = other DNA
ccaaggatcatgcaaccag

SEQ ID NO: 20 - DNA, 20 bp, synthetic construct
  source 1..20: mol_type = other DNA
gcaggtatcccgaaaggtct

SEQ ID NO: 21 - DNA, 23 bp, synthetic construct
  source 1..23: mol_type = other DNA
cttcagttcgtgtgtggagacag

SEQ ID NO: 22 - DNA, 16 bp, synthetic construct
  source 1..16: mol_type = other DNA
cgccctccgactgctg

SEQ ID NO: 23 - DNA, 18 bp, synthetic construct
  source 1..18: mol_type = other DNA
gaataacacagccgatgg

SEQ ID NO: 24 - DNA, 18 bp, synthetic construct
  source 1..18: mol_type = other DNA
ccaggtcactatcaggag

SEQ ID NO: 25 - DNA, 18 bp, synthetic construct
  source 1..18: mol_type = other DNA
caccaactgggacgacat

SEQ ID NO: 26 - DNA, 18 bp, synthetic construct
  source 1..18: mol_type = other DNA
acagcctggatagcaacg

SEQ ID NO: 27 - DNA, 19 bp, synthetic construct
  source 1..19: mol_type = other DNA
tatggcgacccgcagccct

SEQ ID NO: 28 - DNA, 21 bp, synthetic construct
  source 1..21: mol_type = other DNA
catctcgagcaagacgttcag